(19) United States
(12) Patent Application Publication
(10) Pub. No.: US 2007/0172957 A1
(43) Pub. Date: Jul. 26, 2007

(54) Methods of identifying anti-inflammatory macrolides

(75) Inventors: Christie Fanton, Emeryville, CA (US); Mary Lee Mackichan, San Francisco, CA (US); Kiyoshi Nakazawa, Toshima-ku (JP); Daisuke Uchida, Toshima-ku (JP); Kiyoshi Takayama, Toshima-ku (JP)

Correspondence Address: BUCHANAN, INGERSOLL & ROONEY PC, POST OFFICE BOX 1404, ALEXANDRIA, VA, 22313-1404, US

(73) Assignee: TAISHO PHARMACEUTICAL CO., LTD., Toshima-ku (JP)

(21) Appl. No.: 11/505,929

(22) Filed: Aug. 18, 2006

Related U.S. Application Data
(63) Continuation-in-part of application No. PCT/US05/05401, filed on Feb. 18, 2005.
(60) Provisional application No. 60/545,177, filed on Feb. 18, 2004.

(51) Int. Cl. G01N 33/92

(52) U.S. Cl. 436/71

(57) ABSTRACT

The invention relates to the screening and selection of macrolide compounds for use as anti-inflammatory agents. The screening and selection of anti-inflammatory macrolides is based on the differential expression of one or more genes involved in the inflammatory process.

### CROSS-REFERENCE TO RELATED APPLICATIONS

The present application is a continuation-in-part of PCT international application No. PCT/US2005/005,401 filed Feb. 18, 2005 and designating the U.S., which claims benefit of U.S. Provisional Application Ser. No. 60/545,177 filed Feb. 18, 2004, the contents of which are incorporated hereinto by reference.

### FIELD OF THE INVENTION

The invention relates to methods for identifying macrolides having properties for reducing inflammation and associated sequalae (e.g., fibrosis).

### BACKGROUND OF THE INVENTION

Macrolide antibiotics having 14- or 15-member lactone rings are known to have anti-inflammatory properties in patients with chronic airway inflammation. There is need in the art for methods of identifying other macrolides with anti-inflammatory properties. Such macrolides may be useful for treating airway or other inflammation and associated conditions (e.g., cystic fibrosis).

### BRIEF SUMMARY OF THE INVENTION

The invention provides at least the following embodiments:

1. A method of screening test macrolides to identify potential anti-inflammatory agents, comprising:

1) contacting a first biological sample with a test macrolide; and

2) detecting, as an alteration, a first level of expression or activity of a target molecule involved in inflammation in the first biological sample relative to a second level of expression or activity of the target molecule involved in inflammation in a second biological sample that has not been contacted with the test macrolide.

2. The method of embodiment 1, wherein a test macrolide that alters the first level of alteration of expression or activity of the target molecule involved in inflammation relative to the second level of alteration of expression or activity of the target molecule involved in inflammation identifies the test macrolide as a potential anti-inflammatory agent.

3. The method of embodiment 1, wherein the alteration of the target molecule in 2) above is an alteration in the level of de novo arachidonic acid biosynthesis.

4. The method of embodiment 3, wherein a macrolide compound that increases or decreases the level of de novo arachidonic acid biosynthesis is selected.

5. The method of embodiment 4, wherein the level of de novo arachidonic acid biosynthesis is detected by measuring incorporation of labeled arachidonic acid into phospholipids.

6. The method of embodiment 4, wherein the level of de novo arachidonic acid biosynthesis is detected by measuring incorporation of label into an intermediate in an arachidonic acid biosynthesis pathway.

7. The method of embodiment 1, wherein the alteration of the target molecule in 2) above is an alteration in the level of extracellular matrix decomposition.

8. The method of embodiment 7, wherein a macrolide compound that decreases the level of extracellular matrix decomposition is selected.

9. The method of embodiment 1, wherein the alteration of the target molecule in 2) above is an alteration in the leucocyte infiltration number.

10. The method of embodiment 9, wherein a macrolide compound that decreases the leucocyte infiltration number is selected.

11. The method of embodiment 1, wherein the alteration of the target molecule in 2) above is an alteration in the level of expression of a gene involved in arachidonic acid biosynthesis or activation.

12. The method of embodiment 11, wherein a macrolide compound that increases or decreases the level of expression of the gene is selected.

13. The method of embodiment 11, wherein the gene is at least one type selected from the group consisting of A5 fatty acid desaturase (FADS1), A6 fatty acid desaturase (FADS2), fatty acid elongase, arachidonyl CoA synthetase, cyclooxygenase 2, prostaglandin E receptor EP4, cytosolic phospholipase A2, fatty acid desaturase, prostaglandin E synthase, prostaglandin E receptor EP1, prostaglandin 12 receptor, and secretory phospholipase A2 receptor 1.

14. The method of embodiment 1, wherein the alteration of the target molecule in 2) above is an alteration in the level of expression of a gene involved in extracellular matrix decomposition.

15. The method of embodiment 14, wherein a macrolide compound that increases the level of expression of the gene is selected.

16. The method of embodiment 15, wherein the gene is at least one type selected from the group consisting of serine proteinase inhibitor, clade B (ovalbumin), and member 3 (SERPINB3).

17. The method of embodiment 14, wherein a macrolide compound that decreases the level of expression of the gene is selected.

18. The method of embodiment 17, wherein the gene is at least one type selected from the group consisting of matrix metalloproteinase 12 (MMP12), matrix metalloproteinase 19 (MMP19), matrix metalloproteinase 9 (MMP9), cathepsin C, and cathepsin K.

19. The method of embodiment 1, wherein the alteration of the target molecule in 2) above is an alteration in the level of expression of a gene involved in leucocyte infiltration.

20. The method of embodiment 19, wherein a macrolide compound that decreases the level of expression of the gene is selected.

21. The method of embodiment 19, wherein the gene is at least one type selected from the group consisting of small inducible cytokine subfamily A (Cys-Cys) member 18 (CCL18), small inducible cytokine subfamily B (Cys-X-Cys) member 10 (CXCL10), small inducible cytokine subfamily B (Cys-X-Cys) member 11 (CXCL11), and small inducible cytokine subfamily B (Cys-X-Cys) member 9 (CXCL9).

22. The method of any one of embodiments 11, 14 and 19, wherein expression of the gene is determined by detecting mRNA, protein or protein synthesis.

23. The method of embodiment 1, wherein the alteration of the target molecule in 2) above is an alteration in the activity of an enzyme involved in arachidonic acid biosynthesis.

24. The method of embodiment 23, wherein a macrolide compound that increases or decreases the activity of the enzyme is selected.

25. The method of embodiment 24, wherein the enzyme is at least one type selected from the group consisting of A5 fatty acid desaturase (FADS1), A6 fatty acid desaturase (FADS2), fatty acid elongase, and arachidonyl CoA synthetase.

26. The method of embodiment 1, wherein the alteration of the target molecule in 2) above is an alteration in the activity of an enzyme involved in extracellular matrix decomposition.

27. The method of embodiment 26, wherein a macrolide compound that increases the activity of the enzyme is selected.

28. The method of embodiment 27, wherein the enzyme is at least one type selected from the group consisting of serine proteinase inhibitor, clade B (ovalbumin), and member 3 (SERPINB3).

29. The method of embodiment 26, wherein a macrolide compound that decreases the activity of the enzyme is selected.

30. The method of embodiment 29, wherein the enzyme is at least one type selected from the group consisting of matrix metalloproteinase 12 (MMP12), matrix metalloproteinase 19 (MMP19), matrix metalloproteinase 9 (MMP9), cathepsin C, and cathepsin K.

31. The method of embodiment 1, wherein the alteration of the target molecule in 2) above is an alteration in the activity of a chemokine involved in leucocyte infiltration.

32. The method of embodiment 31, wherein a macrolide compound that decreases the activity of the chemokine is selected.

33. The method of embodiment 32, wherein the chemokine is at least one type selected from the group consisting of small inducible cytokine subfamily A (Cys-Cys) member 18 (CCL18), small inducible cytokine subfamily B (Cys-X-Cys) member 10 (CXCL10), small inducible cytokine subfamily B (Cys-X-Cys) member 11 (CXCL11), and small inducible cytokine subfamily B (Cys-X-Cys) member 9 (CXCL9).

34. The method of embodiment 1, wherein the biological sample comprises cells.

35. The method of embodiment 1, wherein the biological sample comprises a cellular extract.

36. The method of embodiment 1, wherein the contacting in 1) above is carried out in vitro.

37. The method of embodiment 1, wherein the contacting in 1) above is carried out in VIVO.

38. The method of embodiment 1, wherein the contacting in 1) above is carried out in a cell culture.

39. The method of embodiment 38, wherein the cell culture comprises an epithelial cell line.

40. The method of embodiment 38, wherein the epithelial cell line is a macrophage cell line.
41. The method of embodiment 39, wherein the epithelial cell line is A549.
42. The method of embodiment 40, wherein the epithelial cell line is THP-1.
43. The method of embodiment 1, wherein the anti-inflammatory action is an action against inflammation in the lung.
44. The method of embodiment 43, wherein the inflammation in the lung is pulmonary emphysema, bronchitis, pulmonary fibrosis, acute lung injury, viral pneumonia, bacterial pneumonia, or acute respiratory disease syndrome (ARDS).

### BRIEF DESCRIPTION OF THE FIGURES

FIG. 1. Schematic view of the arachidonic acid pathway.

FIG. 2. Overlapping transcriptional responses between arachidonic acid pathway inhibitors and macrolides. FIG. 2A, Arachidonic acid pathway inhibitors; FIG. 2B, Active anti-inflammatory macrolides; FIG. 2C, Active (clarithromycin, roxithromycin) and inactive (josamycin) macrolides; FIG. 2D, Triacsin C and active macrolides (clarithromycin, roxithromycin); FIG. 2E, Indomethacin and active macrolides (clarithromycin, roxithromycin); FIG. 2F, Clarithromycin, triacsin C and indomethacin.

FIG. 3. Inhibition of PGE₂ production by FADS1 inhibitor (CP-74006) and macrolides. FIG. 3A, Inhibition of PGE₂ production by FADS1 inhibitor (CP-74006); FIG. 3B, Inhibition of PGE₂ production by macrolides.

FIG. 4A and FIG. 4B show the cell counts of inflammatory cells (macrophages and neutrophiles) in bronchoalveolar lavage fluids from model mice with tobacco smoke-induced pulmonary emphysema after six months' exposure. FIG. 4A shows the cell count of macrophages. FIG. 4B shows the cell count of neutrophiles.

FIG. 5 shows the results of pulmonary compliance measured in the model mice with tobacco smoke-induced pulmonary emphysema after six month's exposure.

### DETAILED DESCRIPTION OF THE INVENTION

In embodiments below, the screening method for a macrolide compound having anti-inflammatory action includes the following steps.

1) contacting a first biological sample with a test macrolide; and

2) detecting, as an alteration, a first level of expression or activity of a target molecule involved in inflammation in the first biological sample relative to a second level of expression or activity of the target molecule involved in inflammation in a second biological sample that has not been contacted with the test macrolide.

In this screening method, a test macrolide that alters the first level of alteration of expression or activity of the target molecule involved in inflammation relative to the second level of alteration of expression or activity of the target molecule involved in inflammation may be identified as the test macrolide as a potential anti-inflammatory agent.

Specific examples of the alteration of the target molecule in the above-mentioned step 2) include

(i) that related to arachidonic acid biosynthesis,

(ii) that related to extracellular matrix decomposition, and

(iii) that related to leucocyte infiltration.

Specific examples of (i) above include an alteration in the level of de novo arachidonic acid biosynthesis, an alteration in the level of expression of a gene involved in arachidonic acid biosynthesis or activity, and an alteration in the level of activity of an enzyme involved in arachidonic acid synthesis.

Specific examples of (ii) above include an alteration in the level of extracellular matrix decomposition, an alteration in the level of expression of a gene involved in extracellular matrix decomposition, and an alteration in the activity of an enzyme involved in extracellular matrix decomposition.

Specific examples of (iii) above include an alteration in the leucocyte infiltration number, an alteration in the level of expression of a gene involved in leucocyte infiltration, and an alteration in the activity of a chemokine involved in leucocyte infiltration.

Furthermore, in accordance with the screening method of this embodiment, it becomes possible to carry out screening for a macrolide compound useful for, for example, an inflammatory disease of the lung.

Examples of the inflammatory disease of the lung include pulmonary emphysema (COPD), bronchitis, pulmonary fibrosis, acute lung injury, viral pneumonia, bacterial pneumonia, and acute respiratory disease syndrome (ARDS).

In such lung diseases, examples of compounds involved in the inflammation include those described in Categories 1 to 3 below.

Category 1: In inflammatory disease of the lung, decomposition of extracellular matrix forming lung tissue is important, and a compound that controls (decreases) expression or activity thereof can become a therapeutic drug. Category 1 relates to such compounds, and specific examples thereof include serine or cystein proteinase inhibitor (SERPINB3), which is present in the living body. In particular, SERPINB3 localizes in the lung, inhibits the activity of a cathepsin, which is activated by an inflammatory reaction, and suppresses the inflammatory action. As described in Examples below, clarithromycin (CAM) has an effect of enhancing the expression of a SERPINB3 gene.

Category 2: In inflammatory disease of the lung, decomposition of extracellular matrix forming lung tissue is important, and a compound that controls (decreases) expression or activity thereof can become a therapeutic drug. Category 2 relates to such compounds, and specific examples thereof include MMP12, MMP9, MMP19, cathepsin K, and cathepsin C. These compounds are proteinases that are important in lung inflammation. As described in Examples below, CAM suppresses the expression of these proteinase genes, which are induced during inflammatory stimulation.

Category 3: In inflammatory disease of the lung, infiltration of leucocytes (T cells, macrophages, neutrophils, etc.) into lung tissue is important in making sickness chronic and more virulent. Category 3 is a group of chemokines that promote leucocyte infiltration. As described in Examples below, CAM suppresses expression of the chemokine gene, which is induced during inflammatory stimulation. Because of this, it is useful as a therapeutic drug for suppressing leucocyte infiltration.

In the screening method of this embodiment, detecting an alteration, due to contact with a macrolide compound, in the level of expression or the activity of a target molecule involved in inflammation in the lung enables the action of the macrolide compound on the lung inflammatory action to be reliably evaluated and a macrolide compound that alters the level of expression or the activity of the target molecule to be identified. Because of this, it is possible to select a macrolide compound having an anti-inflammatory action as a material that is useful as a therapeutic drug.

Furthermore, with regard to the above-mentioned (i), using microarray-based expression analysis, we have shown that macrolide treatment of lung epithelial cell lines alters expression of multiple components in the pathway leading to arachidonate, prostaglandin, and leukotriene production. Delta 5 and delta 6 desaturases are responsible for generation of arachidonate from linoleic acid in the diet. Arachidonate is then incorporated into phospholipids. Upon stimulation (e.g., by interleukin 1), arachidonate is cleaved from phospholipid by phospholipase A2 (PLA2) and then undergoes further reactions to generate bioactive lipids, i.e., prostaglandins and leukotrienes. Delta 5 desaturase (FADS1) is consistently upregulated in A549 epithelial cells treated with immune-active, but not inactive, macrolides. In addition to FADS1, active macrolides specifically alter expression of genes encoding PLA2, PGE synthase, and PGE2 receptors. The pathway leading from arachidonate to generation of prostaglandins is highly validated as a therapeutic target for anti-inflammatory drugs, such as aspirin, inhibiting cyclo-oxygenases (COX1 and COX2).

Macrolides have been used clinically, and have demonstrated anti-inflammatory activity with few serious side effects, suggesting that other macrolides identified on the basis of arachidonic acid inhibition will also be safe.

Screening Methods

The invention provides methods of identifying macrolides having anti-inflammatory activity. Methods of the invention can be used to develop macrolides with improved activity and increased efficacy to suppress inflammation, or alternatively to identify new structures with desirable properties.

Examples of the first and second biological samples used in screening include cells and cell extracts.

Contacting these biological samples with a macrolide compound may be carried out in vitro or may be carried out in vivo. It may be carried out in a cell culture. Examples of the cell culture here include an epithelial cell line such as A549 and a macrophage cell line such as THP-1.

The case of the above-mentioned (i), that is, the case in which the target molecule is arachidonic acid or a material involved in arachidonic acid biosynthesis, is explained below as an example, and when carrying out measurement with respect to the above-mentioned other target molecules, screening can be carried out in accordance with the method as in the case of (i).

In the case of the above-mentioned (i) related to arachidonic acid biosynthesis, specifically, methods of the embodiment involve assaying whether a test macrolide alters biosynthesis of arachidonic acid, alters the expression or activities of enzymes involved in the biosynthesis or activation of arachidonic acid (e.g., arachidonic acid CoA synthetase, delta-6 desaturase, fatty acid elongase, and delta-5 desaturase, AA-related genes in Tables 2 and 3), or alters the expression of genes identified as predictive for the anti-inflammatory macrolide effect (Appendix E). Assays in which arachidonic acid or gene expression or enzymatic activity is detected can be carried out after contacting a cell with a test macrolide. Any cell line that secretes prostaglandin or produces arachidonic acid can be used. Epithelial and macrophages lines (e.g., A549 or THP-1) are suitable for this purpose; other cell lines can be identified by carrying out assays such as those described in the specific Examples, below. Alternatively, screening assays can be carried out after adding a test macrolide to an in vitro cell system (e.g., a cellular extract or liver microsome preparation) or an in vitro enzyme assay system.

Furthermore, in the case of the above-mentioned (ii) related to extracellular matrix decomposition, the following can be selected as macrolide compounds.

Those that decrease the level of extracellular matrix decomposition;

those that increase the level of expression of a gene involved in extracellular matrix decomposition, such as serine proteinase inhibitor, clade B (ovalbumin), and member 3 (SERPINB3) (Appendix F);

those that decrease the level of expression of a gene involved in extracellular matrix decomposition, such as matrix metalloproteinase 12 (MMP12), matrix metalloproteinase 19 (MMP19), matrix metalloproteinase 9 (MMP9), cathepsin C, and cathepsin K (Appendix F);

those that increase the activity of an enzyme involved in extracellular matrix decomposition, such as serine proteinase inhibitor, clade B (ovalbumin), and member 3 (SERPINB3); or

those that decrease the activity of an enzyme involved in extracellular matrix decomposition, such as matrix metalloproteinase 12 (MMP12), matrix metalloproteinase 19 (MMP19), matrix metalloproteinase 9 (MMP9), cathepsin C, and cathepsin K.

In the case of the above-mentioned (iii) related to leucocyte infiltration, the following can be selected as macrolide compounds.

Those that decrease the leucocyte infiltration number;

with regard to chemokines such as small inducible cytokine subfamily A (Cys-Cys) member 18 (CCL18), small inducible cytokine subfamily B (Cys-X-Cys) member 10 (CXCL10), small inducible cytokine subfamily B (Cys-X-Cys) member 11 (CXCL11), and small inducible cytokine subfamily B (Cys-X-Cys) member 9 (CXCL9), those that decrease the level of expression of a gene involved in leucocyte infiltration (Appendix F); or

with regard to chemokines such as small inducible cytokine subfamily A (Cys-Cys) member 18 (CCL18), small inducible cytokine subfamily B (Cys-X-Cys) member 10 (CXCL10), small inducible cytokine subfamily B (Cys-X-Cys) member 11 (CXCL11), and small inducible cytokine subfamily B (Cys-X-Cys) member 9 (CXCL9), those that decrease the activity of a chemokine involved in leucocyte infiltration.

Test Macrolides

“Test macrolides” according to the invention include any macrolide desired to be tested for potential anti-inflammatory activity. Test macrolides may or may not be known to have anti-bacterial or other activities. Macrolides known to have anti-inflammatory activity can be screened in methods of the invention, for example to screen such macrolides for relative potency. Screening methods of the invention also can be used to assess relative potency after modification of test macrolides.

Preferred test macrolides have 14- or 15-member lactone rings and can be natural products or semi-synthetic derivatives thereof. Representatives of macrolides having 14-member lactone rings are the erythromycins, produced from Streptomyces erythreus, such as erythromycin A, erythromycin B, erythromycin C, erythromycin D, erythromycin E, erythromycin estolate, erythronolid, and clarithromycin. Other examples of macrolide antibiotics with 14-member lactone rings include megalomycin and its derivatives, picromycin, narbomycin, oleandomycin, triacetyl-oleandomycin; and the neutral compounds laukamycin, kujimycin A, albocyclin, and cineromycin B. Examples of macrolides having 15-member lactone rings include azalides, such as azithromycin.

Test macrolides can be synthesized using methods well known in the art. Methods of synthesizing macrolides are disclosed, for example, in U.S. Pat. Nos. 4,921,978; 4,933,474; 4,963,582; 5,023,348; 5,034,542; 5,116,968; and 6,100,239.

Typically, a test macrolide identified as a potential anti-inflammatory agent either (1) increases or decreases biosynthesis of arachidonic acid intermediates or products by at least 20, 30, 40, 50, 60, 70, 80, 90, 100, 150, or 200% or more, (2) alters (i.e., increases or decreases) expression of a gene in the arachidonic acid biosynthesis pathway by at least 1.3-fold as determined by methods described in the specific examples, below, or (3) alters (i.e., increases or decreases) enzymatic activity of an enzyme in the arachidonic acid biosynthesis pathway by at least 10, 30, 40, 50, 60, 70, 80, 90, 100, 150, or 200% or more.

Assays for Decreased Arachidonic Acid Production

Quantitative in vitro or in vivo assays can be used to determine whether arachidonic acid production decreases in response to a test macrolide, e.g., by measuring a decrease in radiolabeled arachidonic acid or measuring an increase or decrease of radiolabeled precursors, as is known in the art. Free arachidonic acid can be measured, for example, as described in U.S. Pat. No. 5,663,053. Alternatively, liver microsomes can be incubated in the presence of radiolabeled linoleic acid or other precursor, and the production of labeled arachidonic acid can be detected, as is known in the art. Alternatively, incorporation of label into phospholipids containing arachidonic acid could be measured. See, e.g., Obukowicz et al., Biochem. Pharmacol. 55, 1045-58, 1998; Obukowicz et al., J. Pharm. Exptl. Ther. 287, 157-66, 1998. This type of assay can readily be adapted to high throughput screening (see below).

Determining Gene Expression

Either mRNA or protein can be detected to determine gene expression. mRNA can be detected a variety of methods well known in the art, including, but not limited to, RNA dot blots, slot blots, Northern blots, RT-PCR (including TaqMan RT-PCR), SAGE (serial analysis of gene expression), and electrochemical detection of nucleic acid hybridization (e.g., U.S. Pat. No. 6,361,951). See also US 20030096782; Liang & Pardee, Science 257, 967, 1992; Higuchi et al., BioTechnology 10, 413-17, 1992, and Higuchi et al., BioTechnology 11, 1026-30, 1993; Holland et al., Proc. Natl. Acad. Sci. U.S.A. 88, 7276-80, 1991; Heid et al., Genome Res. 6, 986-94, 1996; and Gibson et al., Genome Res. 6, 995-1001, 1996. Alternatively, mRNA products can be identified by contacting RNA with one or more oligonucleotide or cDNA probes unique to the FADS1, FADS2, or fatty acid (elongase) gene, either in solution or immobilized on a solid support, such as a membrane or a gene expression “chip.” Such chips can be purchased commercially (e.g., from Affymetrix or Motorola) or can be custom made.

Protein can be detected using a variety of techniques known to the art, including immunochemical methods such as radioimmunoassay, Western blotting, and immunohistochemistry. Alternatively, protein synthesis can be determined in vivo, in a cell culture, or in an in vitro translation system by detecting incorporation of labeled amino acids into protein products.

Measuring Individual Enzyme Activity

Various assays are available to measure the activities of delta-6 desaturase (FADS2), fatty acid elongase, delta-5 desaturase (FADS1), and arachidonyl CoA synthetase. For delta-6 desaturase, these include, but are not limited to, assays described in U.S. Pat. No. 6,492,108 and references disclosed therein. For fatty acid elongase, assays include those described in Chang et al., J. Nutr. 122, 2074-80, 1992; U.S. Pat. No. 6,307,128; and US 20030104596. For FADS1 enzyme activity, assays include those described in de Gomez Dumm et al., Lipids 18, 781-88, 1983; Leikin & Brenner, Biochim. Biophys. Acta 963, 311-19, 1988; Chanussot et al., Diabetolgia 32, 786-91, 1989; Venkatesan et al., Biomed. Chromatography 4, 234-38, 1990; Maniongul et al., Lipids 28, 291-97, 1993; Su & Brenna, Anal. Biochem. 261, 43-50, 1998; Leonard et al., Biochem. J. 347, 719-24, 2000; Nishida et al., J. Pineal Res. 32, 26-33, 2002. Obukowicz et al., J. Pharm. Exptl. Ther. 287, 157-66, 1998, describes quantitative in vitro or in vivo assays for both delta-6 and delta-5 desaturase activities. See also Obukowicz et al., Biochem. Pharmacol. 55, 1045-58, 1998. Arachidonyl CoA synthetase activity can be measured as described, for example, in Bhat & Block, Am. J. Physiol. 262, L606-13, 1992; Beaumelle & Vial, Biochim. Biophys. Acta 958, 1-9, 1988; or Gerritsen & Perry, Biochim. Biophys. Acta 1045, 174-79, 1990.

Measuring Level of Extracellular Matrix Decomposition

An inflammatory substance (e.g. lipopolysaccharide, inflammatory cytokine, tobacco smoke, hydrochloric acid, anticancer agent) is pulmonarily or systemically administered to an animal. After administration of the inflammatory substance, lung lavage fluid or serum of an animal to which administration of the inflammatory substance has been carried out or has not been carried out is prepared. The amount of desmosine that is solubilized accompanying extracellular matrix decomposition is measured by the ELISA method (Laurent P et. al. J Immunol Methods. 1988 Feb. 24; 107(1): 1-11.)

Alternatively, the amount of solubilized hydroxyproline is measured in accordance with a standard method.

Measuring Leucocyte Infiltration Number

An inflammatory substance (e.g. lipopolysaccharide, inflammatory cytokine, tobacco smoke, hydrochloric acid, anticancer agent) is pulmonarily or systemically administered to an animal. After administration of the inflammatory substance, lung lavage fluid is recovered from a mouse to which administration of the inflammatory substance has been carried out or has not been carried out, and by counting the leucocyte infiltration number, the leucocyte infiltration number accompanying the inflammation can be measured.

Furthermore, after administration of the inflammatory substance, a lung pathological specimen is prepared from a mouse to which administration of the inflammatory substance has been carried out or has not been carried out, and by counting the number of cells that are stained by an antibody that recognizes leucocytes (e.g. MAC-1), the leucocyte infiltration number accompanying the inflammation can be measured.

Measuring Chemokine Activity

An inflammatory substance (e.g. lipopolysaccharide, inflammatory cytokine, tobacco smoke, chemical substance, anticancer agent) is pulmonarily or systemically administered to an animal. After administration of the inflammatory substance, a lung lavage fluid and a soluble fraction of lung lysate are prepared from an animal to which administration of the inflammatory substance has been carried out or has not been carried out. Alternatively, an inflammatory substance is added to cultured cells, and the cell supernatant is prepared to give a chemokine-containing liquid.

This chemokine-containing liquid is used to measure the activity of chemokine toward leucocytes by a Boyden chamber assay. Alternatively, the chemokine-containing liquid is contacted with highly chemokine receptor-expressing cells, and a transient increase in the intracellular Ca concentration is detected to give an activity.

High Throughput Screening

Using high throughput screening, many discrete test macrolides can be tested in parallel so that large numbers of test macrolides can be quickly screened. The most widely established techniques utilize 96-well microtiter plates. The wells of the microtiter plates typically require assay volumes that range from 50 to 500 μl. In addition to the plates, many instruments, materials, pipettors, robotics, plate washers, and plate readers are commercially available to fit the 96-well format. Alternatively, “free format assays,” or assays that have no physical barrier between samples, can be used. See, e.g., Jayawickreme et al., Proc. Natl. Acad. Sci. U.S.A. 19, 1614-18, 1994. Another high throughput screening method is described in Beutel et al., U.S. Pat. No. 5,976,813. In this method, test samples are placed in a porous matrix. One or more assay components are then placed within, on top of, or at the bottom of a matrix such as a gel, a plastic sheet, a filter, or other form of easily manipulated solid support. When samples are introduced to the porous matrix they diffuse sufficiently slowly, such that the assays can be performed without the test samples running together.

All patents, patent applications, and references cited in this disclosure are expressly incorporated herein by reference. The above disclosure generally describes the present invention. A more complete understanding can be obtained by reference to the following specific examples, which are provided for purposes of illustration only and are not intended to limit the scope of the invention.

### EXAMPLE 1

Identification of Differentially Expressed Genes

Microarray experiments were carried out to identify genes that are differentially expressed in response to various immune stimuli in the presence or absence of macrolides. The data for each cell line (A549 and THP-1) are presented in Appendices A and B. For each cell line, the data for all of the hybridizations are represented together in the same table. For each hybridization, there are two columns: the weighted mean ratio (wtd mean ratio) and the minimum fold change (min fold change). The “min fold change” represents the minimum change in expression that meets the 95% confidence threshold. It can be estimated from the log ratio and standard deviation, assuming that the uncertainty in the ratio is normally distributed. Note that the minimum fold change can never be less than one (hence the minimum function). See also Cheng Li and Wing Hung Wong, “Model-based analysis of oligonucleotide arrays: model validation, design issues and standard error application,” Genome Biology 2001 2(8):research0032.1-0032.11 (published 3 Aug. 2001).

The analysis method to generate these tables was similar for A549 and THP-1, cells. All of the significance threshold criteria were set for the “min fold change” column, which ensured that the resulting genes would be differentially expressed by greater than the threshold value with 95% confidence (p value <0.05). The lowest significance threshold used was 1.3-fold, because it has been demonstrated that a 1.25-fold threshold has a false positive rate of less than 1 in 1000. It is important to note that the fold change threshold is an absolute number, and the direction of change (increased or decreased expression) must be determined by the looking at the weighted mean ratio. The significance threshold criteria are included in parentheses in each “min fold change” column. The weighted mean ratios representing significant differentials (95% confidence, p value <0.05) are in bold. The “Spot IDs” correspond to publicly available cDNA sequences for the region of the gene represented at that spot on the microarray chip.

For the A549 and THP-1 cell lines (Appendix A and B, respectively), the first table for each cell line (“total differentials”) represents all of the genes that show differential expression in at least one of the hybridizations. The second table for A549 and THP-1 cells (“drug effects, sum ≧4.5” and “drug effects, sum ≧5”, respectively) has an additional final column, “sum of min fold change.” This column represents the sum of the minimum fold changes for all of the groups except the stimulus alone. This column was used to rank the genes in order of the greatest global drug effect, independent of whether the drug used was Clarithromycin or FMA9045 and independent of stimulus effect. A sum min fold change of greater than or equal to 4.5 indicates that at least one of the four groups (Clari alone, FMA9045 alone, Clari+stimulus, FMA+stimulus) had a minimum fold change of at least 1.5.

For A549 cells, the tables of Appendix A include genes that met one or more of the following criteria: greater than 1.5-fold change for IL-1β stimulation and greater than 1.3-fold change for each of the drug groups. As noted above, a sum min fold change ≧4.5 was used as the threshold for ranking the most highly regulated drug effects. In both tables in Appendix A, the genes that meet this criterion (sum ≧4.5) are in bold, and those genes that are bold and italics are the most highly regulated genes and have a min fold change ≧5.0.

For THP-1 cells, the tables of Appendix B include genes that met one or more of the following criteria: greater than two-fold change for LPS stimulation, greater than 1.5-fold change for clarithromycin alone and for clarithromycin+/−LPS, and greater than 1.3-fold change for FMA9045 alone and for FMA9045+/−LPS. For THP-1 cells, a sum min fold change ≧5.0 was used as the threshold for ranking the most highly regulated drug effects. In both tables in Appendix B, the genes that meet this criterion (sum ≧5) are in bold and in italics. A larger sum min fold change threshold was used for THP-1 cells, because the macrolide response in this cell line was more robust than that in A549 cells.

Results Overview

The global transcription response to the various culture conditions is shown in Table 1, below. The min fold change threshold (noted next to each condition) was used to determine the total number of significant differentially expressed genes (95% confidence).

TABLE 1

| | | Min fold | Total # Differential |
|---|---|---|---|
| | | change | Genes |
| Cell line | Condition | threshold | (95% confidence) |
| A549 | +/−IL-1β | 1.5 | 185 |
| | +/−Clari | 1.3 | 117 |
| | +/−FMA | 1.3 | 20 |
| | IL-1 +/− Clari | 1.3 | 127 |
| | IL-1 +/− FMA | 1.3 | 16 |
| THP-1 | +/−LPS | 2.0 | 223 |
| | +/−Clari | 1.5 | 127 |
| | +/−FMA | 1.3 | 22 |
| | LPS +/− Clari | 1.5 | 120 |
| | LPS +/− FMA | 1.3 | 25 |

The results of the microarray experiments in the A549 cell line reveal that a large number of genes (>175) are regulated in response to IL-1β stimulation. Similarly, in the THP-1 macrophage line, a large group of genes (>200) are regulated in response to LPS stimulation. While these numbers of differentially expressed genes are similar, it is important to note that the min fold change threshold was set lower for the IL-1, stimulus response in A549 cells (1.5), as compared to a threshold of 2.0 for the LPS stimulus response in THP-1 cells. This is because the transcriptional response to stimulus was larger in THP-1 cells than in A549 cells.

In cells treated with clarithromycin alone, a similar number of genes were differentially expressed in each cell line (117 genes in A549 cells and 127 genes in THP-1 cells). In cells treated with stimulus (IL-1β or LPS)+clarithromycin, the number of differentially expressed genes also was similar (127 genes in A549 and 120 genes in THP-1). As with the stimulus alone condition, however, the min fold change threshold was set lower for the A549 cells for both the clarithromycin alone and the stimulus+/−clarithromycin conditions (1.3 for A549 versus 1.5 for THP-1). Again, this is because the global transcription response was not as robust in the A549 cells as it was in the THP-1 cells.

In both A549 and THP-1 cells, the response to FMA9045 treatment was much weaker than the response to Clarithromycin. In cells treated with FMA9045 alone, a similar number of genes were differentially expressed in both cell lines (20 genes in A549 cells and 22 genes in THP-1 cells). In response to stimulus (IL-1β or LPS)+FMA9045, 16 genes were differentially expressed in A549 cells, and 25 genes were differentially expressed in THP-1 cells. In contrast to the stimulus alone and the Clarithromycin conditions, the min fold change threshold was set at 1.3 for both A549 and THP-1 cells, because the overall response to FMA9045 was relatively weak.

### EXAMPLE 2

Functions Associated with Macrolide Target Genes

Genes that were differentially expressed in one or more of the macrolide treatment groups were categorized into several groups, as shown in Tables 2 and 3. A selection of representative genes is included under each category, with the full number of differentially expressed genes in each category in parentheses next to the category name. Similar patterns were apparent in both the A549 and THP-1 cell lines, although the particular genes in each category were often different. The macrolide-regulated genes considered for these tables are from the “drug effects” tables in Appendices A and B. As discussed in Example 1, genes in these tables were ranked in order of their sum min fold change, and only the most highly regulated genes that were above a certain threshold were included. For THP-1 cells, genes that had a sum min fold change above 5.0 were included, whereas for A549 cells, genes that had a sum min fold change above 4.5 were included.

In THP-1 cells (Table 2), the largest number of responsive genes were those encoding the cytokines and chemokines (27 total). Interestingly, a large number of genes fell into the global category of tissue/matrix remodeling (30 total). Genes in this category included many protease-related genes (matrix metalloproteinases, thrombin-related genes), as well as structural/matrix genes (fibronectin and collagen) and EGF-related genes. Importantly, the expression of the matrix metalloproteinases was (in most cases) decreased in the presence of stimulus plus macrolide (compared to stimulus alone), while the expression of the metalloproteinase inhibitor TIMP2 was increased in the same treatment group, suggesting coordinate regulation. The signaling cascade of phospholipase A2 to prostaglandin E-related genes (arachidonic acid-related signaling) was another notable set of differentially expressed genes. In this case, PTGS2 and PLA2G4A showed increased expression in response to stimulus and then an additional increase in expression in response to stimulus plus macrolide.

TABLE 2
Macrolide Responsive Genes in THP1 Macrophage Cells (sum >5.0)

| General Function/ | | |
|---|---|---|
| Pathway (total gene #) | Gene | Gene Description |
| Tissue/Matrix | | |
| remodeling (30) | | |
| Structural/matrix (9) | FN1 | Fibronectin |
| | *CTGF | Connective tissue growth factor |
| | Col7A1 | Collagen type 7, alpha 1 |
| | *PLOD | Lysyl hydroxylase |
| Protease-related (15) | *MMP3 | Matrix metalloproteinase 3 |
| | MMP7 | Matrix metalloproteinase 7 |
| | *MMP10 | Matrix metalloproteinase 10 |
| | TIMP2 | Metalloprotease inhibitor 2 |
| | *PLAU | Plasminogen activator, urokinase |
| | *F2 | Thrombin |
| | *THBS1 | Thrombospondin |
| | THBD | Thrombomodulin |
| EGF-related (6) | *AREG | Amphiregulin |
| | *NRG1 | Neuregulin |
| | *DTR | HB-EGF |
| | *EREG | Epiregulin |
| | *FGF2 | Fibroblast growth factor 2 |
| | FGFR1 | Fibroblast growth factor receptor |
| Arachidonic Acid- | *PTGS2 | Cyclooxygenase 2 |
| related sigenaling (3) | PTGER4 | Prostaglandin E receptor, EP4 |
| | *PLA2G4A | Cytosolic phospholipase A2 |
| GPCR-related | *RGS14 & 16 | Regulators of G-protein |
| signaling (7) | | signaling |
| | *GRIN2C | Subunit of NMDA receptor |
| | GNG11 | G protein gamma subunit 11 |
| | GNAS | G protein alpha subunit s |
| | ADCY7 | Adenylate cyclase 7 |
| Chemokine/Cytokine | *IL8 | Interleukin 8 |
| signaling (27) | *IL1β | Interleukin 1β |
| | ECSIT | TIR adaptor |
| | CD14 | LPS binding protein |
| | *SCYA20 | MIP3a |
| | *SCYA3 | MIP1α |

| | |
|---|---|
| *LPS regulated | Genes responsive in both THP-1 and A549 cells |

In A549 cells (Table 3), the global transcriptional response to the various treatment conditions was not as robust as that seen in THP-1 cells, but the differentially expressed genes were in similar categories to those seen in THP-1 cells. Similar to THP-1 cells, a large number of genes were in the global category of tissue/matrix remodeling (26 total). There was quite a bit of overlap in the differentially expressed tissue/matrix remodeling-related genes between A549 and THP-1 cells, including several protease-related genes (MMP7, TIMP2, THBS1), and multiple EGF ligands (AREG, EREG, NRG). Also similar to THP-1 cells, genes involved in arachidonic acid signaling were differentially expressed in A549 cells (including FADS1, PTGES, PTGER1, and PLA2G4B), although the specific genes did not overlap with the differentially expressed genes in THP-1 cells. Similar to the observation in THP-1 cells, these arachidonic acid-related genes were up-regulated in response to macrolide. Up-regulation of gene expression is a common signaling feedback response following inhibition of enzyme(s) activity in that signaling pathway.

TABLE 3
Macrolide Responsive Genes in A549 Epithelial Cells (sum >4.5)

| General Function/ | | |
|---|---|---|
| Pathway (total gene #) | Gene | Gene Description |
| Tissue/Matrix | | |
| remodeling (26) | | |
| Structural/matrix (12) | LAMC2 | laminin |
| | P4HA2 | Prolyl-hydroxylase alpha 2 subunit |
| | PLOD | Lysyl hydroxylase |
| | *CTGF | Connective tissue growth factor |
| | *Col4A2 | Collagen type 4, alpha 2 |
| Protease-related (10) | *MMP7 | Matrix metalloproteinase 7 |
| | TIMP2 | Metalloprotease inhibitor 2 |
| | *PLAU | Plasminogen activator, urokinase |
| | *PLAUR | Urokinase plasminogen |
| | | activator rec. |
| | *SerpinE1 | Plasminogen activator inhibitor |
| | THBS1 | Thrombospondin |
| EGF-related (4) | *AREG | Amphiregulin |
| | *EREG | Epiregulin |
| | NRG1 | Neuregulin |
| | FRAG1 | FGF receptor activating protein 1 |
| Arachidonic Acid- | FADS1 | Fatty acid desaturase |
| related signaling (7) | | (catalyzes AA production) |
| | *PTGES | Prostaglandin E synthase |
| | PTGER1 | Prostaglandin E receptor, EP1 |
| | PTGIR | Prostaglandin I2 receptor |
| | *PLA2G4B | Cytosolic phospholipase A2 |
| | PLA2R1 | Secretory phospholipase |
| | | A2 receptor 1 |
| GPCR-related | *BDKRB1 | Bradykinin B1 receptor |
| signaling (4) | GNB5 | G protein beta 5 subunit |
| | GPRK7 | G-protein coupled receptor |
| | | kinase 7 |
| | *P2RY1 | P2Y purinergic receptor |
| Chemokine/Cytokine | *IL-8 | Interleukin 8 |
| signaling (8) | *SCYA20 | MIP3a |
| | ECSIT | TIR adaptor |
| | SARM | TIR domain |

| | |
|---|---|
| *IL-1β regulated | Genes responsive in both THP-1 and A549 cells |

### EXAMPLE 3

Macrolide-dependent changes in gene expression in THP-1 and A549 cell lines fall into several functional categories which are relevant to airway inflammation, including tissue/matrix remodeling and arachidonic acid metabolism. Previous microarray results suggested a number of possible target signaling pathways, including thrombin/PAR-1, TGFβ, bradykinin B2 receptor, PGE₂, and arachidonic acid (AA) biosynthesis/metabolism. Experiments were then designed to determine which of these pathways was most likely to be the actual signaling pathway targeted by macrolides. In addition, a microarray experiment was carried out with THP-1 macrophages to examine the effect of macrolides on a larger number of genes on multiple microarray slides. The microarray data demonstrated that thrombin, TGFβ, and PGE₂ signaling pathways were not likely the direct targets of macrolide activity, although TGF-β may be indirectly affected.

The data from the initial microarray experiments suggested that either the PGE₂ signaling pathway or the AA pathway might be a target of macrolides. The PGE₂ signaling pathway is downstream of AA metabolism, and cells respond to PGE₂ stimulation by changes in cAMP or Ca levels.

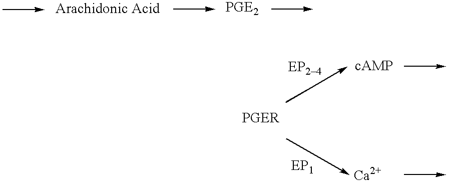

A number of genes involved in PGE₂ biosynthesis from AA (PLA₂, PGE synthase/PTGES), as well as the PGE receptor (PGER) EP1 and EP4 subtypes, were differentially expressed in response to macrolides, suggesting the PGE₂ pathway as a possible macrolide target. However, in preliminary experiments, neither A549 nor THP-1 cells showed PGE-dependent changes in cAMP levels. This data suggested that it was unlikely that macrolides were inhibiting PGER-dependent signaling if the cells were not responsive to PGE₂. An additional experiment demonstrated, however, that macrolide treatment caused a small, but reproducible, decrease in PGE₂ production, in response to IL-1β stimulation. This indicates that macrolides have a regulatory effect on the AA pathway.

The AA pathway can be split into AA biosynthesis and AA metabolism. In the biosynthetic portion of the pathway, AA is synthesized from lipid in the diet and incorporated into phospholipids. AA metabolism involves AA release from phospholipids, usually in response to immune stimulation, and subsequent generation of a variety of lipid products, including prostaglandins and leukotrienes.

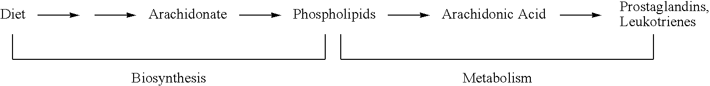

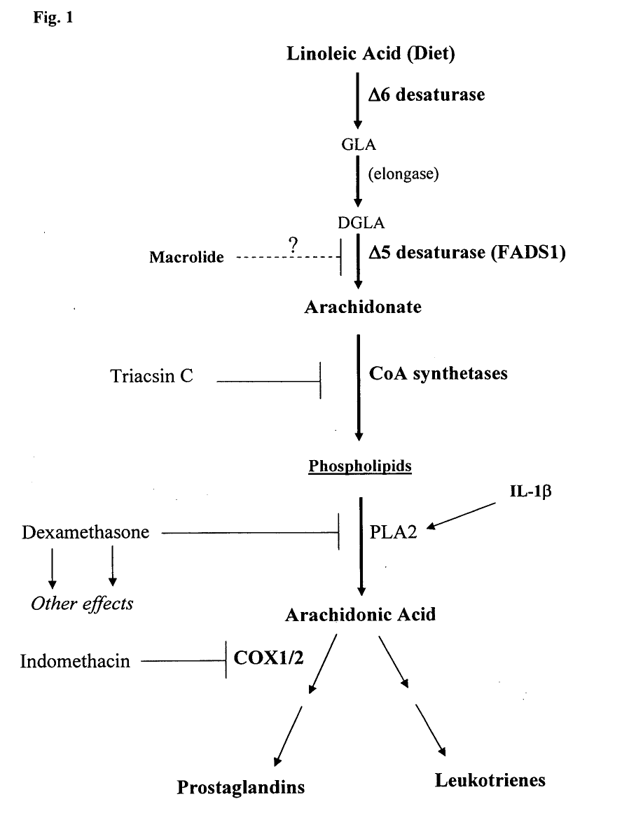

FADS1 is a key enzyme in the biosynthesis of AA (FIG. 1), and FADS1 was differentially expressed in response to 50 μM clarithromycin in every A549 array experiment. These data suggest that the AA pathway might be the macrolide target pathway and FADS1 might be a molecular target of macrolides in lung epithelial cells.

Prostaglandins (including PGE₂) and leukotrienes are inflammatory modulators generated from AA. This pathway is attractive as a target of macrolide anti-inflammatory activity, because interference with this pathway is well validated therapeutically by inhibitors of COX1 and 2. In addition to prostaglandins and leukotrienes, other lipid products generated from AA include prostacyclins (from COX enzymes), thromboxanes (from COX), and HETEs (from 12- and 15-lipoxygenases).

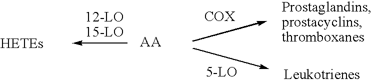

To better understand the ability of macrolides to regulate the AA signaling pathway, a microarray experiment was carried in A549 cells to compare the transcriptional response of macrolides to that of several inhibitors of the AA pathway (FIG. 1). One inhibitor, triacsin C, acts on AA biosynthesis by inhibiting arachidonyl CoA synthetases, blocking the incorporation of free AA into phospholipids. Triacsin C is reasonably specific for AA compared to other fatty acids. However, triacsin C structurally resembles AA so it also may interfere with other steps in the AA pathway. Indomethacin is an inhibitor of COX1 and COX2 enzyme activities and therefore specifically blocks metabolism of AA into prostaglandins. Dexamethasone is a glucocorticoid and has the broadest effects of these inhibitors. It inhibits transcription of PLA₂, and transcriptionally and post-transcriptionally regulates COX1 and COX2 activities. Dexamethasone has additional effects unrelated to AA through binding with the glucocorticoid receptor to glucocortcoid response elements.

A549 cells were pre-incubated overnight (17 hours) with macrolides (10 μM FMA-9045, 50 μM clarithromycin, 50 μM roxithromycin, 50 μM josamycin) and 15 μM triacsin C, and pre-incubated 2 hours with 100 nM indomethacin and 100 nM dexamethasone. All cells then were stimulated for 4 hours with IL-1β, because the activation of PLA₂ and downstream signaling events generally requires an inflammatory stimulus. The various treatment conditions are shown below in Table 4. Following macrolide and inhibitor pre-treatment and IL-1 stimulation, the cells were lysed, total RNA was collected, and the poly-A+ mRNA fraction was purified. Competitive hybridizations to the microarray were performed as shown in Table 5. The resulting microarray data (Appendix C) were analyzed using the methods described in the Examples above.

TABLE 4
Cell treatments for A549 microarray experiment

| | 4 hr stim | O/N pre-incubation | | | | 2 hr | O/N | 2 hr |
|---|---|---|---|---|---|---|---|---|
| Culture | IL1β | FMA | Clari | Josa | Roxi | Indo | TriacsinC | Dex |
| condition | (10 ng/ml) | (10 uM) | (50 uM) | (50 uM) | (50 uM) | (100 nM) | (15 uM) | (100 nM) |
| 1 | − | − | − | − | − | − | − | − |
| 2 | + | − | − | − | − | − | − | − |
| 3 | + | + | − | − | − | − | − | − |
| 4 | + | − | + | − | − | − | − | − |
| 5 | + | − | − | + | − | − | − | − |
| 6 | + | − | − | − | + | − | − | − |
| 7 | + | − | − | − | − | + | − | − |
| 8 | + | − | − | − | − | − | + | − |
| 9 | + | − | − | − | − | − | − | + |
| 10 | + | − | − | − | + | − | + | − |
| 11 | + | − | + | − | − | − | + | − |

TABLE 5
Hybridizations for A549 microarray experiment

| Treatment Conditions | Hybridization Outcome |
|---|---|
| Untreated vs. IL-1β | IL-1β effect (Inflammation) |
| IL1 vs. IL1 + FMA | FMA-9045 effect |
| IL1 vs. IL1 + Clari | Clarithromycin effect |
| IL1 vs. IL1 + Roxi | Roxithromycin effect |
| IL1 vs. IL1 + Josa | Josamycin effect |
| IL1 vs. IL1 + Indo | Indomethacin effect |
| IL1 vs. IL1 + Triacsin C | Triacsin C effect |
| IL1 vs. IL1 + Dex | Dexamethasone effect |
| IL1 + Triacsin vs. IL1 + | Triacsin effect on Clari response |
| Triacsin + Clari | |
| IL1 + Triacsin vs. IL1 + | Triacsin effect on Roxi response |
| Triacsin + Roxi | |

Results

The tables in Appendix C represent the effects of the arachidonic acid pathway inhibitors (“AA pathway inhibitors”) and the effects of the macrolides (“IL1+macrolide effects”). The first table (“AA pathway inhibitors”) represents genes that show differential expression (>1.3-fold) in response to at least one of the inhibitors. The second table (“all macrolides”) represents genes that show differential expression (>1.3-fold) in response to at least one of the macrolides. In both tables in Appendix C, the genes that meet this criterion (≧1.3 min fold change) are indicated in bold.

The global transcriptional response to the various culture conditions is shown in Table 6. The min fold change threshold was used to determine the total number of significant differentially expressed genes (95% confidence).

TABLE 6
Global transcriptional response

| | Conc. of | Min fold | Total # Differential |
|---|---|---|---|
| | macrolide | change | Genes |
| Condition | (μM) | threshold | (95% confidence) |
| +/− IL-1β | — | 2.0 | 102 |
| IL1 + Clari | 50 | 1.3 | 54 |
| IL1 + Roxi | 50 | 1.3 | 65 |
| IL1 + FMA | 10 | 1.3 | 65 |
| IL1 + Josa | 50 | 1.3 | 73 |
| IL1 + Triacsin C | — | 1.3 | 228 |
| IL1 + Indomethacin | — | 1.3 | 37 |
| IL1 + Dexamethasone | — | 1.3 | 341 |
| IL1 + Triacsin + Clari | 50 | 1.3 | 4 |
| IL1 + Triacsin + Roxi | 50 | 1.3 | 7 |

The main focus of the analysis was to look at the similarities between the response to AA pathway inhibitors and the response to macrolides. It was anticipated that there would be an overlap in the transcriptional responses if the macrolides and the AA pathway inhibitors were acting on the same pathway. In contrast, if macrolides act on a pathway other than the AA pathway, the response to macrolides and the response to the inhibitors should be dissimilar. Additionally, an inhibitor that acts further upstream in a signaling pathway might be expected to affect more genes than an inhibitor acting lower in the pathway.

Arachidonic Acid Biosynthetic Pathway Inhibitors

A small number of genes responded to indomethacin treatment (37), while a larger number of genes responded to triacsin C treatment (228). This is consistent with the idea that inhibitors acting upstream in a pathway will influence transcription of more genes than inhibitors of final steps in a pathway. Triacsin interferes with the biosynthetic portion of the AA pathway by inhibiting AA incorporation into phospholipids. Triascin may also affect other steps in the AA pathway due to its structural similarity to AA, but this has not been demonstrated. Indomethacin inhibits AA metabolism induced by various stimuli by blocking COX enzyme activity, and thus PGE₂ production. Dexamethasone affects multiple steps in the AA pathway as well as other unrelated pathways. The number of genes responding to dexamethasone was quite large (341), as predicted based on the multiple transcriptional targets of glucocorticoid receptors.

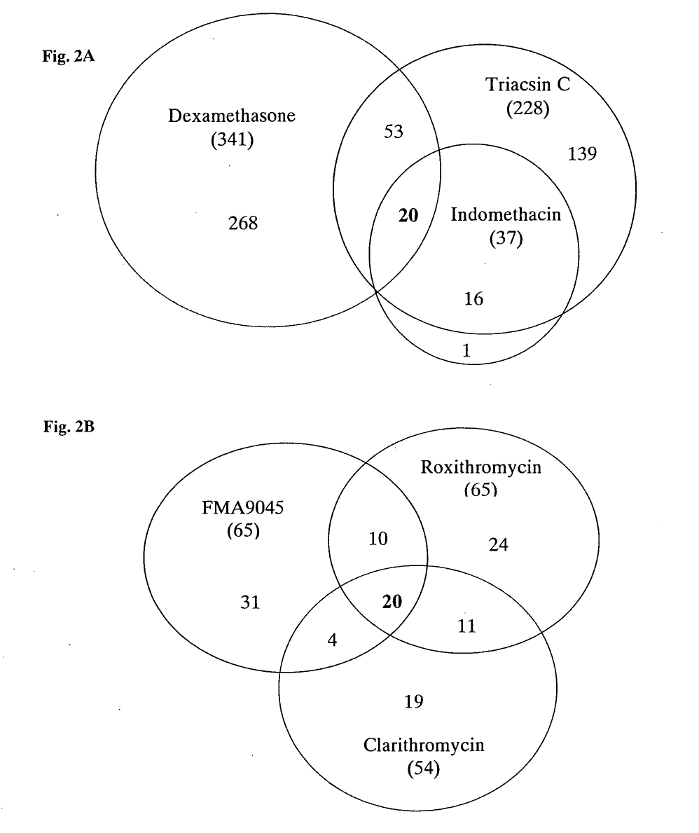

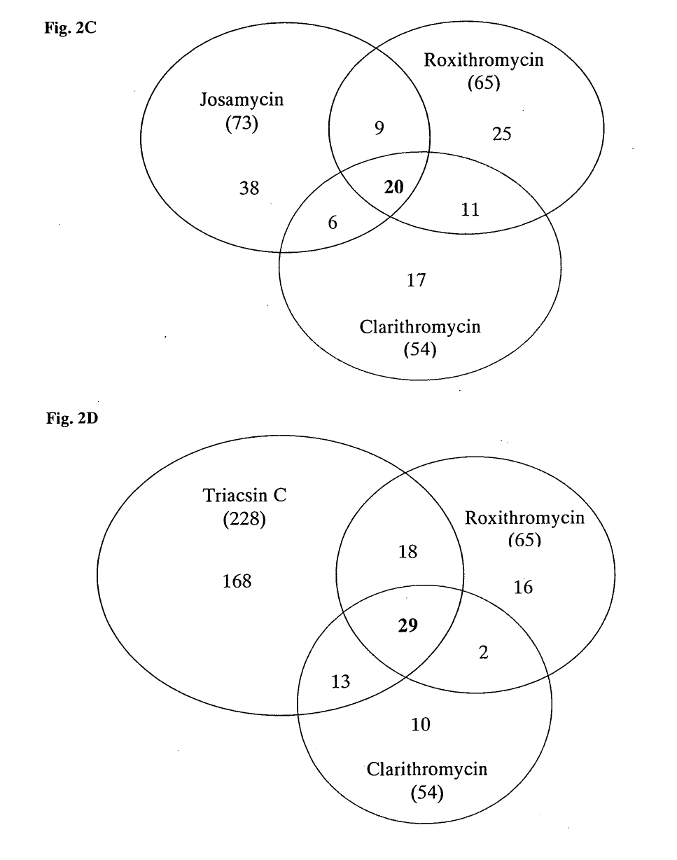

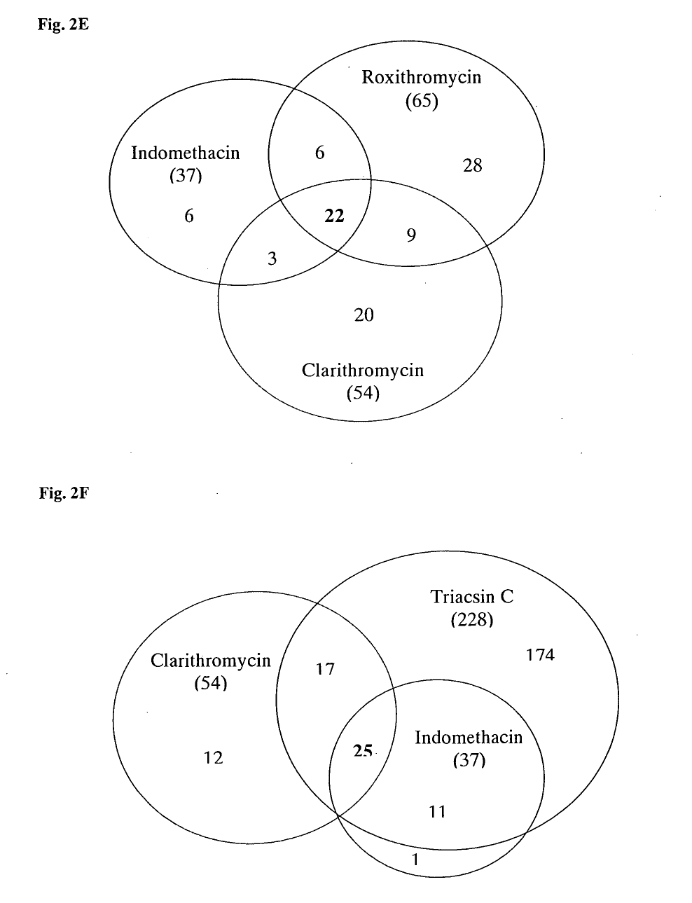

Many genes were differentially regulated in response to more than one of the AA pathway inhibitors. The overlap of the responses is shown in a Venn diagram, in which numbers represent genes similarly regulated by the corresponding treatments (FIG. 2A). As shown in FIG. 2, there are 20 genes that are differentially regulated by all three AA pathway inhibitors (triacsin C, indomethacin, and dexamethasone). These 20 genes can be considered the core transcriptional response to manipulation of the AA pathway.

Looking at overlap between pairs of inhibitors, the indomethacin-dependent response is almost entirely a subset of the triacsin C response: 37 genes were differentially expressed in response to indomethacin, and 36 of these 37 genes were differentially expressed in response to both triacsin C and indomethacin. This is not unexpected, considering the known mechanism of action of these inhibitors. Indomethacin inhibits COX1 and COX2, and therefore acts downstream of triacsin C on the AA biosynthetic pathway (FIG. 1). It could be anticipated that the indomethacin response would be a subset of the response to triacsin C, and this is what was observed. Indomethacin specifically affects production of prostaglandins, but triacsin C inhibits AA incorporation into phospholipids, affecting production of other lipid products (i.e., leukotrienes, prostacyclins). This interference upstream of branchpoints in the AA pathway leading to lipid products other than PGE₂ by triascin C leads to further changes in transcription not seen with indomethacin.

In contrast to the strong overlap between triacsin C and indometbacin, the overlap between the response to dexamethasone and the response to triacsin C and indomethacin is smaller. Some overlap was expected, because dexamethasone does affect the AA pathway. However, dexamethasone is a more general inhibitor, affecting pathways other than the AA pathway, so there are also many other genes affected by dexamethasone presumably through other pathways.

Quantitative analysis was done to determine how well the responses to the different AA pathway inhibitors correlated. The correlation values were assigned a p-value to provide statistical confidence to the correlation value. Correlation between experiments, across all spots showing good detection, was assessed by cosine vector angle to generate the data shown in Tables 7-9. A correlation coefficient close to one indicates strong correlation, whereas a value near zero indicates no correlation. P-values for each correlation coefficient were estimated based on a Fisher permutation test. In this test, each spot's expression ratios were randomly re-ordered across experiments and the degree of correlation between experiments was then reassessed. This was done repeatedly. The p-value is the fraction of these random reorderings that result in a larger cosine vector angle than was observed for the unpermuted data. This method of assessing significance was the most conservative of several that were tested. Using Pearson correlation coefficient instead of cosine vector angle also gave equivalent results.

This analysis further supports the conclusions drawn from the amount of gene overlap between the different responses. The dexamethasone response is not strongly correlated with the response to either triacsin C (0.196, p-value 0.1) or indomethacin (0.266, p-value 0.009), reflecting its additional transcriptional targets outside the AA pathway. In contrast, the AA pathway inhibitors triacsin C and indomethacin show a significant correlation (0.485, p-value <0.001), consistent with their shared effect on PGE₂ biosynthesis. A related analysis, which only used the subset of genes differentially expressed in response to triacsin C for the correlation assessment, showed an even stronger correlation between triacsin C and indomethacin (0.621, p-value 0.1), while the correlation with the dexamethasone response was still low and was not significant (0.281, p-value 1.0).

TABLE 7
Similarity and significance of AA pathway inhibitor responses
(all spots) Values in boldface represent the correlations and
p-values of interest (the other numbers represent inhibitors
correlated with themselves or duplicate correlations).
Correlation coefficients are shown, with p-values for the
correlation coefficient in parentheses. A p-value of
1.00 means that the correlation is not significant.

| | IL-1β | Dexamethasone | Triacsin C | Indomethacin |
|---|---|---|---|---|
| Dexamethasone | −0.608 | Not applicable | 0.196 | 0.266 |
| | (1.000) | | (0.103) | (0.009) |
| Triacsin C | −0.035 | 0.196 | Not | 0.485 |
| | (1.000) | (0.103) | applicable | (<0.001) |
| Indomethacin | −0.195 | 0.266 | 0.485 | Not applicable |
| | (1.000) | (0.009) | (<0.001) | |

Overlap Between Macrolide Responses

The genes differentially expressed in the presence of clarithromycin, FMA-9045, and roxithromycin overlapped moderately (FIG. 2B), as observed in previous experiments. It is not surprising that the responses to the active macrolides diverge somewhat. While there is much existing literature reporting that both clarithromycin and roxithromycin have anti-inflammatory activity in vivo, their anti-inflammatory efficacy varies in relation to each other, depending on the report. However, because our experiments have shown that the global response to these active macrolides can be represented by the same functional gene classes, it is expected that these 14-member macrolides are acting by a similar mechanism.

In this experiment, the transcriptional response to the active macrolides (clarithromycin and roxithromycin) also overlapped with the transcriptional response to josamycin (see Venn diagram, FIG. 2C). Josamycin is a 16-member macrolide and has not been reported to have anti-inflammatory activity. However, due to the related structure of the 14- and 16-member macrolides, it is not unexpected that the transcriptional response to josamycin would overlap somewhat with the response to the active macrolides. It may be that the overlapping response between josamycin and the actives represents a basic response to the macrolide class, but that the transcriptional responses that are unique to the active macrolides represent the genes or pathways involved in the specific 14-member macrolide anti-inflammatory activity. Importantly, FADS1 was differentially expressed in response to active macrolides (clarithromycin and roxithromycin) but not in response to josamycin.

As with the AA pathway inhibitors, the correlation analysis of the different macrolides (Table 8) reinforces the observed overlapping gene response. All the macrolides are well correlated with one another, and these correlation coefficients are statistically highly significant (p-values <0.001).

TABLE 8
Similarity and significance of macrolide responses (all spots)

| | IL-1β | Clari | FMA | Roxi | Josa |
|---|---|---|---|---|---|
| Clari | −0.059 | Not | 0.430 | 0.683 | 0.601 |
| | (1.000) | applicable | (0.000) | (0.000) | (0.000) |
| FMA | −0.154 | 0.430 | Not | 0.658 | 0.660 |
| | (1.000) | (<0.001) | applicable | (0.000) | (0.000) |
| Roxi | −0.049 | 0.683 | 0.658 | Not | 0.721 |
| | (1.000) | (<0.001) | (<0.001) | applicable | (0.000) |
| Josa | 0.060 | 0.601 | 0.660 | 0.721 | Not |
| | (0.967) | (<0.001) | (<0.001) | (<0.001) | applicable |

Overlap Between Pathway Inhibitors and Macrolide Responses

An experiment was carried out to address whether macrolides affect the arachidonic acid pathway. The similarity or dissimilarity of macrolide and AA pathway inhibitor transcriptional profiles should reflect the degree to which they have similar effects on the pathway. The results support the hypothesis that macrolides inhibit the AA pathway.

The transcriptional response to clarithromycin and roxithromycin overlapped significantly with the response to triacsin C and indomethacin (FIGS. 2D and 2E). There is a larger overlap between macrolides and triacsin C than between macrolides and indomethacin. Forty-two of the 54 genes differentially expressed in response to clarithromycin were also differential in response to triacsin C, but only 25 clarithromycin-regulated genes also were differentially expressed in response to indomethacin (FIG. 2F). Triacsin C acts on AA biosynthesis, by inhibiting incorporation of AA into phospholipids. The target of triacsin C is one step downstream of FADS1 (FIG. 1), the proposed target of macrolides. Indomethacin is further downstream on the AA pathway, inhibiting COX enzymes in the stimulus-dependent metabolism part of the AA pathway. If the macrolide mechanism of action is similar to indomethacin (i.e., COX inhibition) the responses to macrolide are predicted to resemble the indomethacin response. However, the macrolide response is actually more similar to the triacsin C effect. Further, FADS1 expression is induced by treatment with either macrolide or triacsin C and not by indomethacin or dexamethasone. The significant overlap between macrolides and triacsin C, as well as the similar regulation of FADS1 expression, tends to support the idea that the target of macrolides is at, or upstream from, the step inhibited by triacsin C, and is acting on AA biosynthesis.

The quantitative analysis of the correlations between the response to active macrolides and the response to AA pathway inhibitors (Table 9) demonstrates that responses to all 3 active macrolides are significantly correlated with those to both triacsin C and indomethacin. Responses to clarithromycin and roxithromycin are most highly correlated with the triacsin C response. The effects of macrolides on epithelial cells are most similar to those of triacsin C and indomethacin, inhibitors targeting the AA pathway, supporting the hypothesis that the AA pathway is a likely macrolide target.

TABLE 9
Similarity and significance of macrolide and
AA pathway inhibitor responses

| | IL-1β | Dexamethasone | Triacsin C | Indomethacin |
|---|---|---|---|---|
| Clari | −0.059 | 0.162 | 0.607 | 0.553 |
| | (1.000) | (0.295) | (<0.001) | (<0.001) |
| FMA | −0.154 | 0.269 | 0.552 | 0.588 |
| | (1.000) | (0.008) | (<0.001) | (<0.001) |
| Roxi | −0.049 | 0.188 | 0.681 | 0.639 |
| | (1.000) | (0.137) | (<0.001) | (<0.001) |

A similar analysis was performed using only the subset of genes differentially expressed in response to clarithromycin for the correlation assessment. The subset analysis demonstrates that clarithromycin-induced responses are significantly correlated with triacsin C (0.916, p-value 0.001). The correlation of clarithromycin with indomethacin for this subset of genes was less significant (0.848, p-value 0.07), and little correlation was observed with dexamethasone (0.430, p-value 1.0).

Effect of Triacsin C on Macrolide Response

If it is true that macrolides act on the AA pathway, then chemically inhibiting the pathway is expected to block the effects of macrolides on AA-related gene expression. For example, if the macrolide target is FADS1, then inhibition of AA biosynthesis with triascin should result in a similar gene expression profile with or without the addition of macrolide. This is true because FADS1 is upstream of the step blocked by triascin. The ability of macrolides to alter gene expression in the presence of triascin was tested directly by competitive hybridization of IL1+Triacsin vs. IL1+Triacsin+Clari/Roxi (Table 7, mRNA from culture conditions 8 v. 10 or 11). It is important to remember that results of direct competitive hybridizations are a ratio of gene expression changes from one group versus changes from the other group—i.e., a 10-fold increase in gene X in both IL1+Triacsin and IL1+Triacsin+Clari would result in no observed change in the gene expression ratio (ratio=1) when those groups were compared in a direct competitive hybridization.

There were two possible outcomes from these two final hybridizations. Outcome #1 would be that triacsin C and macrolides act on different signaling pathways. In this case, the AA pathway inhibitor and macrolides would have different effects on transcription and the transcriptional response should resemble the response to macrolide alone, because the IL1+Triacsin in both groups would effectively cancel out the response to triacsin.

Outcome #1: IL1+Triacsin/IL1+Triacsin+ML=ML

A second possibility, Outcome #2, would be that the AA pathway inhibitor (triacsin C) and macrolides are acting on the same signaling pathway. In this case, the transcriptional response to IL1+Triacsin+macrolide would resemble the response to IL1+Triacsin alone, because the macrolide effect would be blocked by or overlap the triacsin effect. The overall observed response to the direct competitive hybridization in Outcome #2 would be no transcriptional changes, since the ratio of IL1+Triacsin vs. IL1+Triacsin+macrolide (resembling triacsin alone) would be 1.

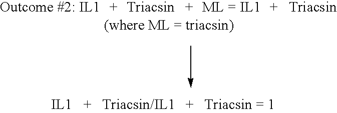

In the hybridization of IL1+Triacsin vs. IL1+Triacsin+Clari, there are only 4 differentially expressed genes across the 2 conditions, in contrast to 54 differentially expressed genes in the comparison of IL1 alone to IL1+clarithromycin (Table 7, culture conditions 2 vs. 4). In a similar direct competitive hybridization between IL1+Triacsin and IL1+Triacsin+Roxi, there were only 7 differentially expressed genes (Table 7, conditions 8 v. 10), compared to 65 differentially expressed genes in response to roxithromycin in the absence of triascin (conditions 2 v. 6).

The results of these hybridization experiments are compelling. The macrolide effect is blocked by, or is overlapping, the triacsin C effect. The minimal change in gene expression in these direct competitive hybridizations corresponds to outcome #2. These data imply that triacsin C blocks the macrolide effect or that the two effects are nearly identical, and strongly support the hypothesis that the AA pathway is a target of macrolides. These results further suggest that the molecular target is likely to be upstream of triacsin C in the biosynthetic pathway, which is comprised of just four possible target enzymes and includes FADS1.

FADS1 Antisense and Inhibitor Microarray Experiment

FADS1, also known as Δ5 desaturase, is the enzyme responsible for conversion of dihomo-gamma-linolenic acid (DGLA) to AA. The related gene FADS2 is A6 desaturase, and is responsible for an earlier step in the biosynthesis of AA—the conversion of linoleic acid (from the diet) to gamma-linolenic acid (GLA). The function of a third related gene, FADS3, is unclear, however it is believed to be more closely related to FADS2 than FADS1.

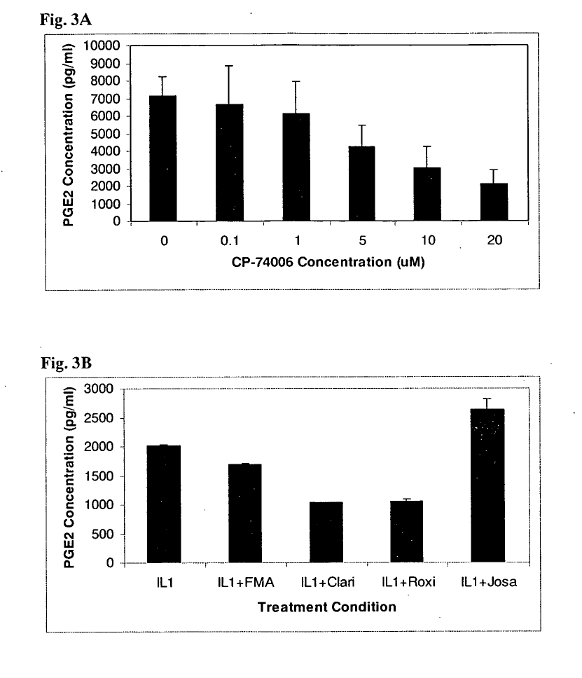

Experiments in which A549 cells are exposed to a compound reported to be a specific inhibitor of FADS1 activity reduced expression of PGE₂ production, as determined by competitive enzyme immunoassay (FIG. 3A). This reduction in PGE₂ has been observed in response to macrolide treatment (FIG. 3B), and further supports the possibility that the specific macrolide target in the AA biosynthetic pathway is FADS1.

To specifically address whether macrolides were specifically targeting the FADS1 enzyme of the arachidonic acid biosynthesis pathway, a microarray experiment was carried out in A549 cells to compare the transcriptional response of macrolides to that of an inhibitor reported to be specific for FADS1 (CP-74006) and to the response of FADS1 mRNA knock-down by antisense oligonucleotides.

A549 cells were transfected with 2 different pairs of FADS1 antisense/reverse control oligonucleotides and incubated for 48 hours. Cells then were pre-incubated overnight (17 hours) with macrolides (50 μM clarithromycin, 50 μM roxithromycin), and 10 μM CP-74006. All cells then were stimulated for 4 hours with IL-1β, because the activation of PLA₂ and downstream signaling events generally requires an inflammatory stimulus. The various treatment conditions are shown below in Table 10. Following macrolide and inhibitor pre-treatment and IL-1β stimulation, the cells were lysed, total RNA was collected, and the poly-A+ mRNA fraction was purified. Competitive hybridizations to the microarray were performed as shown in Table 11. The resulting microarray data was analyzed using the methods described in the Examples above. The transcriptional response to CP74006 (FADS1 inhibitor) is in Appendix D.

The table in Appendix D represents the effects of the FADS1 inhibitor in the absence and presence of macrolides, and represents genes that show differential expression (>1.3-fold) in response to at least one of the conditions. The genes that meet this criterion (≧1.3 min fold change) are indicated in bold.

TABLE 10
Treatments for antisense, FADS1 inhibitor microarray experiment

| | | | | | | | | FADS1 |
|---|---|---|---|---|---|---|---|---|
| Culture | | | | AS | RC | | | (CP- |
| Con- | | AS | RC | 262- | 262- | Clari | Roxi | 74006) |
| dition | IL1β | 262-6 | 262-6 | 12 | 12 | (50 μM) | (50 μM) | (10 μM) |
| 1 | − | − | − | − | − | − | − | − |
| 2 | + | − | − | − | − | − | − | − |
| 3 | + | + | − | − | − | − | − | − |
| 4 | + | − | + | − | − | − | − | − |
| 5 | + | − | − | + | − | − | − | − |
| 6 | + | − | − | − | + | − | − | − |
| 7 | + | + | − | − | − | + | − | − |
| 8 | + | − | + | − | − | + | − | − |
| 9 | + | − | − | + | − | + | − | − |
| 10 | + | − | − | − | + | + | − | − |
| 11 | + | − | − | − | − | + | − | − |
| 12 | + | − | − | − | − | − | + | − |
| 13 | + | − | − | − | − | − | − | + |
| 14 | + | − | − | − | − | + | − | + |
| 15 | + | − | − | − | − | − | + | + |

TABLE 11
Hybridizations for antisense, FADS1 inhibitor microarray experiment

| Treatment Conditions | Hybridization Outcome |
|---|---|
| Untreated vs. IL-1β | IL-1β effect (Inflammation) |
| IL1 vs. IL1 + Clari | Clarithromycin effect |
| IL1 vs. IL1 + Roxi | Roxithromycin effect |
| IL1 vs. IL1 + FADS1 inhibitor | FADS1 inhibitor effect |
| IL1 + FADS1 inh vs. IL1 + | Clan effect in presence of FADS1 inhib |
| FADS1 inh + Clari | |
| IL1 + FADS1 inh vs. IL1 + | Roxi effect in presence of FADS1 inhib |
| FADS1 inh + Roxi | |
| RC-6 vs. RC-6 + Clari | Clari effect w/RC background |
| RC-12 vs. RC-12 + Clari | Clari effect w/RC background |
| AS-6 vs. RC-6 | Antisense 6 effect |
| AS-12 vs. RC-12 | Antisense 12 effect |
| AS-6 vs. AS-6 + Clari | Clari effect in presence of KO |
| AS-12 vs. AS-12 + Clari | Clari effect in presence of KO |

Quantitative analysis was done to determine how well the response to the FADS1 inhibitor correlated with the macrolide response and with the responses to the other inhibitors of the arachidonic acid pathway (triacsin C and indomethacin). The macrolide and arachidonic acid pathway inhibitor data used for the correlations are in Appendix C and the FADS1 inhibitor data are in Appendix D. The correlation analysis was done using the methods described in the Examples above.

The analysis indicates a strong correlation between the FADS1 inhibitor and both arachidonic acid inhibitors triacsin and indomethacin. Using the subset of spots differentially expressed in response to triacsin and indomethacin, the correlation between the FADS1 inhibitor and both triacsin and indomethacin was greater than 0.9 (p-value <0.001). This result was anticipated, since all three of the above inhibitors target the arachidonic acid pathway and therefore should have similar effects on cells.

Significantly, the correlation analysis also demonstrates a strong correlation between the FADS1 inhibitor and the active macrolides. Using a subset of spots differentially expressed in response to clarithromycin and roxithromycin, the correlations between the FADS1 inhibitor and clarithromycin was 0.9 (p-value 0.3), between the FADS1 inhibitor and roxithromycin was greater than 0.9 (p-value 0.03), and between the FADS1 inhibitor and FMA was greater than 0.9 (p-value 0.003). These data suggest that the FADS1 inhibitor and the macrolides are acting on the same pathway.

The correlation analyses demonstrate that the responses to the anti-inflammatory macrolides are highly correlated with the responses to three different inhibitors of the arachidonic acid pathway (triacsin C, indomethacin, and FADS1 inhibitor). These data, in conjunction with the ability of macrolides to inhibit production of PGE2, a product of arachidonic acid activation, strongly support the hypothesis that macrolides are targeting the arachidonic acid pathway.

### EXAMPLE 4

In this example, the usefulness of a macrolide compound for inflammatory disease of the lung was evaluated.

### PREPARATION EXAMPLE

| | | |
|---|---|---|
| clarithromycin | 50 | mg |
| lactose | 40 | mg |
| corn starch | 49.75 | mg |
| crystalline cellulose | 17 | mg |
| carmellose calcium | 17 | mg |
| hydroxypropyl cellulose | 5.25 | mg |
| magnesium stearate | 1 | mg |
| total | 180 | mg |

Clarithromycin, lactose, corn starch, crystalline cellulose, and carmellose calcium were uniformly mixed. To this was added a 10% hydroxypropyl cellulose aqueous solution. After blending the mixture, the granules were dried and sieved with a 30M-screen to make uniform granules. Magnesium stearate was added to the granules and it was compressed to form tablets.

The pharmacological action of clarithromycin (improvement to a pulmonary emphysema condition) will be next explained by way of test examples.

Test Example: Pharmacological effect in model mice with tobacco smoke-induced pulmonary emphysema

The model mice with tobacco smoke-induced pulmonary emphysema mimicking human pulmonary emphysema were constructed according to the method as described in Hautamaki, R. D. et al., Science 1997, 277: 2002-2004 or Shapiro, S. D. et al., Am. J. Pathol (2003) 163: 2329-2335. Specifically, C57black/6 female mice of 12-weeks old were exposed to tobacco smoke at two cigarettes per day, 6 days per week for 6 months.

Administration of clarithromycin was carried out orally at the rate of 25, 50 or 100 mg/body weight twice daily (morning and evening) during the tobacco smoke exposure period. The plasma AUC₀₋₈ of clarithromycin obtained when 50 mg/kg was singly administered to the mice was 2.677 μg·hr/ml while the plasma AUC₀₋₈ of clarithromycin obtained when 200 mg of clarithromycin was singly administered to a normal human subject was 8.98 μg·hr/ml. The former concentration was substantially lower than the latter one and was an adequate concentration to be used clinically.

The index of the improvement to the pulmonary emphysema condition followed the mothod as described in Hautamaki, R. D. et al., ibid. or Shapiro, S. D. et al., ibid.

Hematoxylin eosin-stained pathological specimens were prepared from the model mice and ten visual fields under a microscope were randomly selected, where the average sizes of pulmonary alveoli were measured. An increase in the average size of pulmonary alveolus is considered as an index of the destruction of pulmonary alveoli, i.e., the pulmonary emphysema condition.

Table 12 shows the improvement factor of pulmonary emphysema in the model mice with tobacco smoke induced pulmonary emphysema. Six months' smoking (i.e., exposure to tobacco smoke) increased the pulmonary emphysema rate by 20% relative to the non-smoking group (control). The pulmonary emphysema was improved by 47% in the group administered with clarithromycin (25 mg/kg) and by a maximum of up to 87% in the group administered with clarithromycin (50 mg/kg). These results indicate that clarithromysin is effective against pulmonary emphysema (or pulmonary emphysema condition).

TABLE 12

| | pulmonary | | pulmonary | |
|---|---|---|---|---|
| | alveolus size | statistical | emphysema | improvement |
| | (μm) | significance | rate (%) | factor (%) |
| non-smoking | 32.21 ± 0.507 | | 0 | |
| smoking + | 38.51 ± 0.765 | 0.0004 (vs. | 20 | 0 |
| solvent | | non- | | |
| | | smoking) | | |
| smoking + | 35.60 ± 1.425 | 0.1001 (vs. | 10.6 | 47 |
| CAM | | smoking) | | |
| (25 mg/kg) | | | | |
| smoking + | 33.16 ± 0.890 | 0.0046 (vs. | 3 | 85 |
| CAM | | smoking) | | |
| (50 mg/kg) | | | | |
| smoking + | 34.35 ± 0.936 | 0.0092 (vs. | 6.7 | 66.5 |
| CAM | | smoking) | | |
| (100 mg/kg) | | | | |

(mean ± SE)

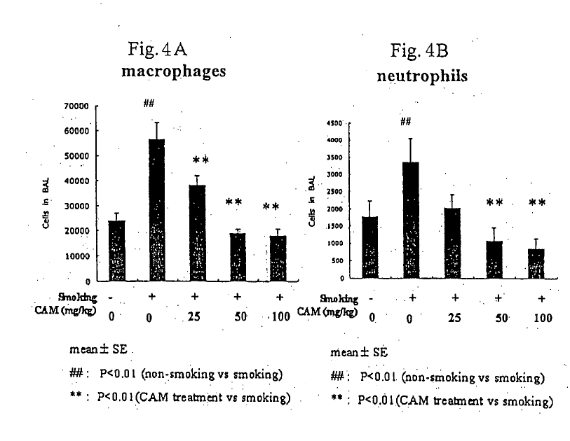

The cell counts of inflammatory cells (microphages and neutrophiles) in the bronchoalveolar lavage fluids after six months' exposure were measured to provide an index of the inflammatory condition in the model mice with tobacco smoke-induced pulmonary emphysema. The results are shown in FIG. 4A and FIG. 4B.

The cell counts of macrophages and neutrophiles increased as the pulmonary emphysema progressed. These cells produce the proteases that are involved in pulmonary emphysema. These cells increase exacerbate the pulmonary emphysema. Administration of clarithromycin reduced the cell counts of macrophages and neutrophiles in a dose-dependant manner. This result indicates that clarithromycin inhibits inflammation reaction and suppresses the progression of pulmonary alveoli destruction.

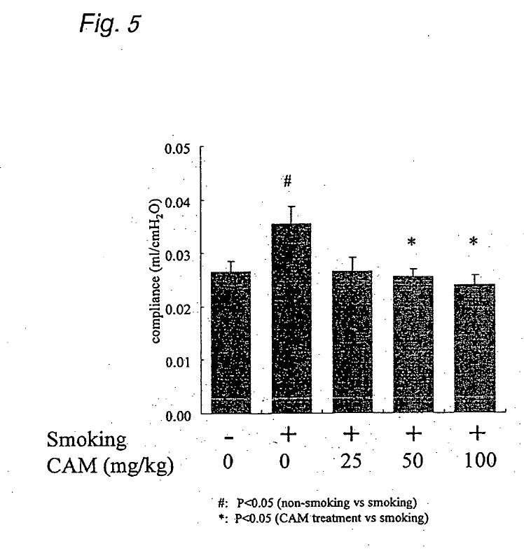

It has been reported that as pulmonary emphysema progresses, pulmonary functions deteriorate. It has also been known that the pulmonary compliance, which is an index of pulmonary functions, increases in model mice with tobacco smoke-induced pulmonary emphysema. Thus, the pulmonary compliance was measured in the model mice with tobacco smoke-induced pulmonary emphysema after six months' exposure. The result is shown in FIG. 5.

As the pulmonary emphysema progressed, the pulmonary compliance (the index of pulmonary functions) was increasing in the model mice with tobacco smoke-induced pulmonary emphysema; however, the pulmonary compliance recovered upon the administration of clarithromycin. This result was in accord with the improvement of the pulmonary emphysema ratio as well as with the decrease in the number of the inflammatory cells, and it indicates that the clarithromycin administration is useful for the treatment of pulmonary emphysema (or pulmonary emphysema condition).

### EXAMPLE 5

In this example, among the genes shown in the above-mentioned Appendixes A to E, the influence of a macrolide compound on the level of expression of a gene of a material related to inflammatory disease of the lung was examined.

The influence of a macrolide compound on the level of expression of the gene of the material related to inflammatory disease of the lung is summarized in Appendix F. In Appendix F, ‘+/−Clari, wtd means’ denotes gene induction (suppression) when clarithromycin is added to THP-1 cells, and the larger the figure, the more the gene is expressed. Furthermore, ‘+LPS+/−Clari, wtd means’ denotes gene induction (suppression) when clarithromycin is added to LSP-stimulated THP-1 cells, and the smaller the figure, the more the gene expression is suppressed.

From Appendix F, it can be seen that clarithromycin acts effectively on the expression of genes of Categories 1 to 3.

The present invention may have the following modes.

1. A method of screening test macrolides to identify potential anti-inflammatory agents, comprising the steps of:
contacting a first biological sample with a test macrolide; and
detecting a first level of de novo biosynthesis of arachidonic acid in the first biological sample, wherein a test macrolide that alters the first level of de novo arachidonic acid biosynthesis relative to a second level of de novo arachidonic acid biosynthesis in a second biological sample that has not been contacted with the test macrolide identifies the test macrolide as a potential anti-inflammatory agent.

2. The method of embodiment 1 wherein the test macrolide increases the first level of de novo arachidonic acid biosynthesis.

3. The method of embodiment 1 wherein the test macrolide decreases the first level of de novo arachidonic acid biosynthesis.

4. The method of embodiment 1 wherein the biological sample comprises cells.

5. The method of embodiment 1 wherein the biological sample comprises a cellular extract.

6. The method of embodiment 1 wherein the biological sample is in an in vivo system.

7. The method of embodiment 1 wherein the biological sample is in an in vitro system.

8. The method of embodiment 1 wherein the in vitro system is a cell culture.

9. The method of embodiment 1 wherein the first level of de novo arachidonic acid biosynthesis is detected by measuring incorporation of labeled arachidonic acid into phospholipids.

10. The method of embodiment 1 wherein incorporation of label into an intermediate in an arachidonic acid biosynthesis pathway is measured.

11. A method of screening macrolides to identify potential anti-inflammatory agents, comprising the steps of:
contacting a first biological sample with a test macrolide; and
determining a first level of expression of a gene involved in arachidonic acid biosynthesis or activation, wherein a test macrolide that alters the first level of expression of the gene relative to a second level of expression of the gene in a second biological sample that has not been contacted with the test macrolide identifies the test macrolide as a potential anti-inflammatory agent.

12. The method of embodiment 41 wherein the test macrolide increases the first level of expression.

13. The method of embodiment 11 wherein the test macrolide decreases the first level of expression.

14. The method of embodiment 11 wherein the step of contacting is in vitro.

15. The method of embodiment 11 wherein the step of contacting is in vivo.

16. The method of embodiment 15 wherein the step of contacting is in a cell culture.

17. The method of embodiment 16 wherein the cell culture comprises an epithelial cell line.

18. The method of embodiment 17 wherein the epithelial cell line is a macrophage cell line.

19. The method of embodiment 17 wherein the epithelial cell line is A549.

20. The method of embodiment 17 wherein the epithelial cell line is THP-1.

21. The method of embodiment 11 wherein the gene is A5 fatty acid desaturase (FADS1).

22. The method of embodiment 11 wherein the gene is A6 fatty acid desaturase (FADS2).

23. The method of embodiment 11 wherein the gene is fatty acid elongase.

24. The method of embodiment 11 wherein the gene is arachidonyl CoA synthetase.

25. The method of embodiment 11 wherein the gene is selected from the group consisting of cyclooxygenase 2, prostaglandin E receptor EP4, cytosolic phospholipase A2, fatty acid desaturase, prostaglandin E synthase, prostaglandin E receptor EP1, prostaglandin 12 receptor, and secretory phospholipase A2 receptor 1.

26. The method of embodiment 11 wherein the gene is selected from the group consisting of genes listed in Appendices A-E.

27. The method of embodiment 11 wherein expression of the gene is determined by detecting mRNA.

28. The method of embodiment 11 wherein expression of the gene is determined by detecting protein.

29. The method of embodiment 11 wherein expression of the gene is determined by detecting protein synthesis.

30. A method of screening macrolides to identify potential anti-inflammatory agents, comprising the steps of:
contacting a first biological sample with a test macrolide; and
determining a first level of activity of an enzyme involved in arachidonic acid biosynthesis, wherein a test macrolide that alters the first level of activity of the enzyme relative to a second level of activity of the enzyme in a second biological sample that has not been contacted with the test macrolide identifies the test macrolide as a potential anti-inflammatory agent.

31. The method of claim 30 wherein the test macrolide increases the first level of activity of the enzyme.

32. The method of claim 30 wherein the test macrolide decreases the first level of activity of the enzyme.

33. The method of embodiment 30 wherein the step of contacting is in vitro.

34. The method of embodiment 30 wherein the step of contacting is in vivo.

35. The method of embodiment 24 wherein the step of contacting is in a cell culture.

36. The method of embodiment 33 wherein the cell culture comprises an epithelial cell line.

37. The method of embodiment 36 wherein the epithelial cell line is a macrophage cell line.

38. The method of embodiment 36 wherein the epithelial cell line is A549.

39. The method of embodiment 36 wherein the epithelial cell line is THP-1.

40. The method of embodiment 30 wherein the enzyme is A5 fatty acid desaturase (FADS1).

41. The method of embodiment 30 wherein the enzyme is A6 fatty acid desaturase (FADS2).

42. The method of embodiment 30 wherein the enzyme is fatty acid elongase.

43. The method of embodiment 30 wherein the enzyme is arachidonyl CoA synthetase.

APPENDIX A

| | | | | | | | | | | | | | | |
|---|---|---|---|---|---|---|---|---|---|---|---|---|---|---|
| | | | | | IL1 | | Clari | | FMA | | IL1b +/− clari, | | IL1b +/− FMA, | |
| | | | | IL1 | treated, | Clari | treated, | FMA | treated, | IL1b +/− clari, | min | IL1b +/− FMA, | min | sum |
| | | | | treated, | min fold | treated, | min fold | treated, | min fold | wtd | fold | wtd | fold | of min |
| | | | | wtd mean | change | wtd mean | change | wtd mean | change | mean | change | mean | change | fold |
| SpotID | GeneSymbol | GeneName | GeneDesc | ratio | (1.5x) | ratio | (1.3x) | ratio | (1.3x) | ratio | (1.3x) | ratio | (1.3x) | change |

Total Differentials in A549 Cells (Genes differential in at least 1 hybridization)
Genes in bold - ≧1.5-fold differential expression in response to IL-1β
Genes in italics - ≧1.3-fold differential expression in response to Clari and/or FMA alone
Genes in bold and italics - ≧1.3-fold differential expression in response to IL1 + Clari and/or IL1 + FMA

| | | | | | | | | | | | | | | |
|---|---|---|---|---|---|---|---|---|---|---|---|---|---|---|
| 49276 | | | | 1.0 | 1.0 | 1.3 | 1.2 | 0.9 | 1.0 | [symbol] | 1.8 | 1.4 | 1.2 | 5.2 |
| 49147 | | | | 1.0 | 1.0 | 1.3 | 1.1 | 0.9 | 1.0 | [symbol] | 1.8 | 1.4 | 1.2 | 5.0 |
| 50600 | | | | 1.0 | 1.0 | 0.7 | 1.4 | 1.1 | 1.0 | 1.0 | 1.0 | 1.2 | 1.1 | 4.5 |
| 49168 | | | | 3.4 | 2.8 | 1.1 | 1.0 | 1.1 | 1.0 | 1.3 | 1.0 | 1.3 | 1.1 | 4.1 |
| 49491 | | | | 2.4 | 1.8 | 1.0 | 1.0 | 1.0 | 1.0 | 1.3 | 1.1 | 0.9 | 1.0 | 4.1 |
| 47030 | | | | 0.6 | 1.4 | 0.6 | 1.3 | 0.9 | 1.1 | [symbol] | 1.5 | 0.9 | 1.0 | 4.8 |
| 48063 | | | | 0.8 | 1.0 | 0.6 | 1.4 | 0.9 | 1.0 | [symbol] | 1.3 | 0.9 | 1.0 | 4.7 |
| 50085 | | | | 2.1 | 2.0 | 1.1 | 1.0 | 1.1 | 1.1 | 1.0 | 1.0 | 1.1 | 1.0 | 4.1 |
| 48739 | | | | 1.7 | 1.4 | 0.8 | 1.0 | 1.1 | 1.0 | [symbol] | 1.7 | 1.0 | 1.0 | 4.7 |
| 46627 | | | | 1.0 | 1.0 | 1.0 | 1.0 | 1.0 | 1.0 | [symbol] | 1.5 | 1.0 | 1.0 | 4.5 |
| 48749 | | | | 1.0 | 1.0 | 1.3 | 1.1 | 1.1 | 1.0 | [symbol] | 1.4 | 1.1 | 1.0 | 4.5 |
| 50278 | | | | 0.8 | 1.2 | 1.0 | 1.0 | 1.1 | 1.0 | [symbol] | 1.4 | 1.0 | 1.0 | 4.4 |
| 47792 | | | | 1.5 | 1.4 | 0.6 | 1.4 | 0.9 | 1.0 | [symbol] | 1.4 | 0.9 | 1.0 | 4.7 |
| 49510 | | | | 1.6 | 1.4 | 1.9 | 1.5 | 1.0 | 1.0 | [symbol] | 1.3 | 1.0 | 1.0 | 4.7 |
| 47373 | | | | 2.2 | 1.5 | 1.2 | 1.1 | 1.0 | 1.0 | 1.3 | 1.2 | 1.1 | 1.0 | 4.4 |
| 46609 | | | | 0.9 | 1.0 | 0.6 | 1.4 | 0.8 | 1.0 | 0.8 | 1.1 | 1.0 | 1.0 | 4.5 |
| 47420 | | | | 1.8 | 1.7 | 0.8 | 1.1 | 1.0 | 1.0 | 0.8 | 1.1 | 1.0 | 1.0 | 4.2 |
| 46704 | | | | 1.0 | 1.0 | 0.9 | 1.0 | 0.7 | 1.3 | 1.0 | 1.0 | 0.9 | 1.0 | 4.3 |
| 46858 | | | | 2.0 | 1.7 | 1.0 | 1.0 | 1.0 | 1.0 | 1.0 | 1.0 | 1.0 | 1.0 | 4.0 |
| 47210 | | | | 0.6 | 1.6 | 0.8 | 1.1 | 0.9 | 1.0 | 0.9 | 1.0 | 0.9 | 1.0 | 4.1 |
| 50306 | | | | 1.6 | 1.6 | 0.9 | 1.0 | 1.0 | 1.0 | 0.9 | 1.0 | 1.0 | 1.0 | 4.0 |
| 49952 | [symbol] 1 | | | 0.9 | 1.0 | 0.6 | 1.3 | 0.8 | 1.3 | 0.7 | 1.0 | [symbol] 3 | 1.4 | 5.0 |
| 47089 | ACYP2 | | | 0.9 | 1.0 | 1.6 | 1.3 | 1.1 | 1.0 | [symbol] 3 | 1.3 | 1.0 | 1.0 | 4.6 |
| 48442 | ADAM17 | TACE | a disintegrin and | 1.8 | 1.7 | 1.1 | 1.0 | 1.0 | 1.0 | 0.9 | 1.0 | 1.0 | 1.0 | 4.0 |
| | | | metalloproteinase | | | | | | | | | | | |
| | | | domain 17 (tum | | | | | | | | | | | |
| 49421 | ADAM17 | TACE | a disintegrin and | 1.7 | 1.6 | 1.2 | 1.1 | 1.0 | 1.0 | 1.0 | 1.0 | 1.0 | 1.0 | 4.1 |
| | | | metalloproteinase | | | | | | | | | | | |
| | | | domain 17 (tum | | | | | | | | | | | |
| 50627 | ADH1A | | | 1.0 | 1.0 | 1.1 | 1.0 | 1.0 | 1.0 | [symbol] 4 | 1.3 | 1.0 | 1.0 | 4.3 |
| 48600 | AGXT | serine- | alanine-glyoxylate | 1.0 | 1.0 | 1.3 | 1.3 | 1.0 | 1.0 | 1.0 | 1.0 | 1.0 | 1.0 | 4.3 |
| | | pyruvate | aminotransferase | | | | | | | | | | | |
| | | aminotransferase | (oxalosis I; h | | | | | | | | | | | |
| 49620 | AKAP1 | | | 0.6 | 1.5 | 1.0 | 1.0 | 1.0 | 1.0 | 1.1 | 1.1 | 0.9 | 1.0 | 4.1 |
| 47127 | ALAD | | | 4.9 | 3.2 | 1.1 | 1.0 | 1.0 | 1.0 | [symbol] | 1.3 | 1.4 | 1.0 | 4.4 |
| 49549 | ANXA8 | | | 1.2 | 1.1 | 0.7 | 1.2 | 1.0 | 1.0 | [symbol] | 1.3 | 0.9 | 1.0 | 4.5 |
| 49935 | APOA2 | | | 2.7 | 2.4 | 1.1 | 1.1 | 1.1 | 1.0 | 1.0 | 1.0 | 1.0 | 1.0 | 4.1 |
| 46982 | AREG | amphiregu-lin | amphiregulin | 2.5 | 2.3 | 0.9 | 1.0 | 1.3 | 1.1 | [symbol] | 1.4 | 1.2 | 1.0 | 4.5 |
| | | | (schwannoma- | | | | | | | | | | | |
| | | | derived growth | | | | | | | | | | | |
| | | | factor) | | | | | | | | | | | |
| 46986 | AREG | amphiregu-lin | amphiregulin | 2.4 | 2.2 | 0.9 | 1.1 | 1.2 | 1.1 | [symbol] | 1.4 | 1.1 | 1.0 | 4.6 |
| | | | (schwannoma- | | | | | | | | | | | |
| | | | derived growth | | | | | | | | | | | |
| | | | factor) | | | | | | | | | | | |
| 46949 | AREG | amphiregu-lin | amphiregulin | 2.6 | 2.4 | 0.9 | 1.0 | 1.3 | 1.1 | [symbol] | 1.3 | 1.1 | 1.0 | 4.4 |
| | | | (schwannoma- | | | | | | | | | | | |
| | | | derived growth | | | | | | | | | | | |
| | | | factor) | | | | | | | | | | | |
| 46953 | AREG | amphiregu-lin | amphiregulin | 2.5 | 2.5 | 1.0 | 1.0 | 1.3 | 1.1 | [symbol] | 1.3 | 1.2 | 1.0 | 4.4 |
| | | | (schwannoma- | | | | | | | | | | | |
| | | | derived growth | | | | | | | | | | | |
| | | | factor) | | | | | | | | | | | |
| 47617 | ARF3 | | | 1.0 | 1.0 | 1.0 | 1.0 | 0.5 | 1.7 | 0.8 | 1.0 | 0.7 | 1.0 | 4.7 |
| 49290 | ARHB | RhoB | ras homolog gene | 1.3 | 1.2 | 1.4 | 1.1 | 1.4 | 1.3 | [symbol] | 1.3 | 1.2 | 1.0 | 4.8 |
| | | | family, member B | | | | | | | | | | | |
| 48282 | ARHG | RhoG | ras homolog gene | 1.8 | 1.6 | 1.3 | 1.2 | 1.1 | 1.0 | [symbol] | 1.4 | 1.2 | 1.1 | 4.7 |
| | | | family, member G | | | | | | | | | | | |
| | | | (rho G) | | | | | | | | | | | |
| 48344 | ARHG | RhoG | ras homolog gene | 1.7 | 1.5 | 1.2 | 1.1 | 1.1 | 1.1 | [symbol] 4 | 1.4 | 1.1 | 1.1 | 4.6 |
| | | | family, member G | | | | | | | | | | | |
| | | | (rho G) | | | | | | | | | | | |
| 50517 | ARHGAP4 | ARHGAP4 | Rho GTPase | 1.1 | 1.0 | 1.2 | 1.1 | 1.0 | 1.0 | [symbol] | 1.4 | 1.0 | 1.0 | 4.5 |
| | | | activating protein 4 | | | | | | | | | | | |
| 46600 | ARHGEF7 | P85SPR | PAK-interacting | 1.6 | 1.5 | 1.0 | 1.0 | 0.9 | 1.0 | 0.9 | 1.0 | 0.9 | 1.0 | 4.0 |
| | | | exchange factor | | | | | | | | | | | |
| | | | beta | | | | | | | | | | | |
| 49155 | [symbol] | | | 0.7 | 1.3 | 0.6 | 1.4 | 0.9 | 1.0 | [symbol] | 1.8 | 1.0 | 1.0 | 5.2 |
| 46817 | ATF3 | ATF3 | activating | 2.3 | 2.0 | 1.3 | 1.3 | 1.4 | 1.1 | [symbol] | 1.3 | 1.0 | 1.0 | 4.7 |
| | | | transcription factor 3 | | | | | | | | | | | |
| 50635 | B4GALT1 | | | 1.9 | 1.8 | 1.5 | 1.1 | 1.0 | 1.0 | 1.3 | 1.2 | 1.0 | 1.0 | 4.3 |
| 49694 | BAG3 | BAG3 | BCL2-associated | 1.2 | 1.0 | 1.1 | 1.0 | 0.9 | 1.0 | [symbol] | 1.3 | 1.0 | 1.0 | 4.3 |
| | | | athanogene 3 | | | | | | | | | | | |
| 48802 | BAI3 | | | 2.2 | 2.1 | 1.3 | 1.1 | 1.0 | 1.0 | 1.3 | 1.2 | 1.1 | 1.0 | 4.3 |
| 47691 | BATF | B-ATF | basic leucine | 0.8 | 1.0 | 0.8 | 1.0 | 0.6 | 1.5 | 0.7 | 1.0 | 0.7 | 1.2 | 4.8 |
| | | | zipper | | | | | | | | | | | |
| | | | transcription | | | | | | | | | | | |
| | | | factor, ATF-lik | | | | | | | | | | | |
| 49013 | BBC3 | | | 2.0 | 2.0 | 1.0 | 1.0 | 1.0 | 1.0 | 1.0 | 1.0 | 1.1 | 1.0 | 4.0 |
| 50033 | BCL10 | BCL10 | B-cell | 1.9 | 1.6 | 1.0 | 1.0 | 1.0 | 1.0 | 1.0 | 1.0 | 0.9 | 1.0 | 4.0 |
| | | | CLL/lymphoma 10 | | | | | | | | | | | |
| 47738 | BCL2A1 | BCL2A1 | BCL2-related | 26.7 | 14.9 | 1.3 | 1.0 | 1.0 | 1.0 | 1.1 | 1.0 | 1.0 | 1.0 | 4.0 |
| | | | protein A1 | | | | | | | | | | | |
| 48697 | BCL3 | Bcl3 | B-cell | 1.5 | 1.3 | 1.1 | 1.0 | 0.9 | 1.0 | [symbol] | 1.4 | 1.0 | 1.0 | 4.4 |
| | | | CLL/lymphoma 3 | | | | | | | | | | | |
| 48679 | BCL3 | Bcl3 | B-cell | 1.0 | 1.0 | 1.1 | 1.0 | 0.6 | 1.5 | 0.9 | 1.0 | 0.8 | 1.0 | 4.5 |
| | | | CLL/lymphoma 3 | | | | | | | | | | | |
| 47979 | BDKRB1 | BDKRB1 | bradykinin | 3.3 | 3.1 | 1.6 | 1.5 | 1.0 | 1.0 | 1.3 | 1.1 | 0.9 | 1.0 | 4.5 |
| | | | receptor B1 | | | | | | | | | | | |
| 48374 | BDKRB1 | BDKRB1 | bradykinin | 4.3 | 3.5 | 1.5 | 1.4 | 0.9 | 1.0 | 1.4 | 1.1 | 1.0 | 1.0 | 4.4 |
| | | | receptor B1 | | | | | | | | | | | |
| 46689 | BENE | | | 0.7 | 1.3 | 1.3 | 1.0 | 1.0 | 1.0 | [symbol] 4 | 1.3 | 1.1 | 1.0 | 4.3 |
| 50726 | BID | BID | BH3 interacting | 2.4 | 2.2 | 0.9 | 1.0 | 1.0 | 1.0 | 1.0 | 1.0 | 0.8 | 1.0 | 4.0 |
| | | | domain death | | | | | | | | | | | |
| | | | agonist | | | | | | | | | | | |
| 48766 | BID | BID | BH3 interacting | 2.4 | 2.2 | 0.9 | 1.0 | 0.9 | 1.1 | 1.0 | 1.0 | 1.0 | 1.0 | 4.1 |
| | | | domain death | | | | | | | | | | | |
| | | | agonist | | | | | | | | | | | |
| 49310 | BID | BID | BH3 interacting | 2.4 | 2.1 | 0.8 | 1.0 | 0.9 | 1.0 | 1.0 | 1.0 | 1.0 | 1.0 | 4.1 |
| | | | domain death | | | | | | | | | | | |
| | | | agonist | | | | | | | | | | | |
| 49061 | BIRC2 | cIAP1 | baculoviral IAP | 2.3 | 2.2 | 1.1 | 1.0 | 0.9 | 1.0 | 0.9 | 1.0 | 0.9 | 1.1 | 4.2 |
| | | | repeat-containing 2 | | | | | | | | | | | |
| 49094 | BIRC2 | cIAP1 | baculoviral IAP | 2.3 | 2.2 | 1.0 | 1.0 | 0.9 | 1.0 | 0.9 | 1.0 | 0.9 | 1.1 | 4.1 |
| | | | repeat-containing 2 | | | | | | | | | | | |
| 48375 | BIRC2 | cIAP1 | baculoviral IAP | 2.1 | 2.0 | 1.0 | 1.0 | 0.9 | 1.0 | 1.0 | 1.0 | 0.9 | 1.1 | 4.1 |
| | | | repeat-containing 2 | | | | | | | | | | | |
| 48408 | BIRC2 | cIAP1 | baculoviral IAP | 2.0 | 2.0 | 1.0 | 1.0 | 0.9 | 1.0 | 1.0 | 1.0 | 1.0 | 1.0 | 4.0 |
| | | | repeat-containing 2 | | | | | | | | | | | |
| 50448 | BIRC3 | cIAP2 | baculoviral IAP | 20.3 | 17.7 | 1.5 | 1.3 | 0.8 | 1.2 | 1.2 | 1.1 | 1.0 | 1.0 | 4.6 |
| | | | repeat-containing 3 | | | | | | | | | | | |
| 47497 | BIRC3 | cIAP2 | baculoviral IAP | 9.9 | 7.2 | 1.2 | 1.0 | 0.9 | 1.0 | 1.1 | 1.0 | 0.9 | 1.0 | 4.0 |
| | | | repeat-containing 3 | | | | | | | | | | | |
| 49065 | BMP4 | BMP4 | bone | 0.8 | 1.1 | 1.3 | 1.0 | 0.9 | 1.0 | [symbol] | 1.6 | 1.0 | 1.0 | 4.6 |
| | | | morphogenetic | | | | | | | | | | | |
| | | | protein 4 | | | | | | | | | | | |
| 49098 | BMP4 | BMP4 | bone | 0.8 | 1.1 | 1.4 | 1.2 | 0.9 | 1.0 | [symbol] | 1.6 | 1.0 | 1.0 | 4.8 |
| | | | morphogenetic | | | | | | | | | | | |
| | | | protein 4 | | | | | | | | | | | |
| 49840 | BMP4 | BMP4 | bone | 1.2 | 1.0 | 1.2 | 1.0 | 0.9 | 1.0 | [symbol] | 1.4 | 1.0 | 1.0 | 4.4 |
| | | | morphogenetic | | | | | | | | | | | |
| | | | protein 4 | | | | | | | | | | | |
| 49981 | BMP6 | BMP6 | bone | 1.4 | 1.1 | 1.5 | 1.4 | 0.9 | 1.0 | [symbol] | 1.5 | 0.8 | 1.1 | 5.0 |
| | | | morphogenetic | | | | | | | | | | | |
| | | | protein 6 | | | | | | | | | | | |
| 50319 | BMP6 | BMP6 | bone | 1.2 | 1.2 | 1.5 | 1.4 | 1.0 | 1.0 | [symbol] 4 | 1.3 | 0.9 | 1.1 | 4.8 |
| | | | morphogenetic | | | | | | | | | | | |
| | | | protein 6 | | | | | | | | | | | |
| 47516 | BTG1 | | | 1.2 | 1.1 | 1.5 | 1.3 | 1.1 | 1.0 | [symbol] | 1.4 | 1.1 | 1.0 | 4.7 |
| 47930 | C21orf4 | C21ORF4 | chromosome 21 | 0.9 | 1.0 | 0.9 | 1.0 | 0.7 | 1.3 | 1.0 | 1.0 | 0.8 | 1.1 | 4.4 |
| | | | open reading | | | | | | | | | | | |
| | | | frame 4 | | | | | | | | | | | |
| 49509 | C3 | | | 2.9 | 2.7 | 0.9 | 1.0 | 1.0 | 1.0 | 0.7 | 1.2 | 0.9 | 1.0 | 4.2 |
| 50709 | C5 | C5 | complement | 0.6 | 1.5 | 1.0 | 1.0 | 1.0 | 1.0 | 1.1 | 1.0 | 0.9 | 1.0 | 4.0 |
| | | | component 5 | | | | | | | | | | | |
| 49928 | C6orf5 | | | 1.8 | 1.7 | 1.0 | 1.0 | 1.1 | 1.0 | 1.2 | 1.1 | 1.0 | 1.0 | 4.1 |
| 48480 | CARS | | | 1.1 | 1.1 | 0.8 | 1.2 | 0.9 | 1.0 | [symbol] | 1.4 | 0.9 | 1.0 | 4.6 |
| 47007 | CASP4 | CASP4 | caspase 4, | 3.0 | 2.3 | 1.4 | 1.4 | 1.0 | 1.0 | 1.0 | 1.0 | 1.1 | 1.0 | 4.4 |
| | | | apoptosis-related | | | | | | | | | | | |
| | | | cysteine protease | | | | | | | | | | | |
| 49807 | CASP4 | CASP4 | caspase 4, | 2.7 | 2.2 | 1.4 | 1.3 | 1.0 | 1.0 | 1.1 | 1.0 | 1.0 | 1.0 | 4.3 |
| | | | apoptosis-related | | | | | | | | | | | |
| | | | cysteine protease | | | | | | | | | | | |
| 50721 | CASP4 | CASP4 | caspase 4, | 3.1 | 2.8 | 1.3 | 1.2 | 1.0 | 1.0 | 1.1 | 1.0 | 1.0 | 1.0 | 4.2 |
| | | | apoptosis-related | | | | | | | | | | | |
| | | | cysteine protease | | | | | | | | | | | |
| 49425 | CASP7 | caspase-7 | caspase 7, | 4.1 | 3.7 | 1.1 | 1.0 | 0.9 | 1.0 | 1.2 | 1.1 | 1.1 | 1.0 | 4.1 |
| | | | apoptosis-related | | | | | | | | | | | |
| | | | cysteine protease | | | | | | | | | | | |
| 48336 | CASP7 | caspase-7 | caspase 7, | 4.3 | 3.5 | 1.0 | 1.0 | 0.9 | 1.0 | 1.1 | 1.0 | 1.1 | 1.0 | 4.0 |
| | | | apoptosis-related | | | | | | | | | | | |
| | | | cysteine protease | | | | | | | | | | | |
| 49909 | CASR | | | 1.6 | 1.5 | 1.2 | 1.1 | 1.1 | 1.0 | 1.3 | 1.2 | 1.1 | 1.0 | 4.3 |
| 49773 | CCNA2 | cyclin A2 | cyclin A2 | 1.0 | 1.0 | 0.5 | 1.4 | 0.9 | 1.0 | 0.6 | 1.2 | 0.8 | 1.1 | 4.8 |
| 49118 | CCNA2 | cyclin A2 | cyclin A2 | 0.9 | 1.0 | 0.6 | 1.3 | 0.9 | 1.0 | [symbol] | 1.5 | 0.8 | 1.0 | 4.8 |
| 49085 | CCNA2 | cyclin A2 | cyclin A2 | 0.9 | 1.0 | 0.7 | 1.1 | 1.0 | 1.0 | [symbol] | 1.4 | 0.8 | 1.0 | 4.5 |
| 48586 | CD4 | CD4 | CD4 antigen (p55) | 0.9 | 1.0 | 0.9 | 1.0 | 0.6 | 1.4 | 0.8 | 1.0 | 0.7 | 1.2 | 4.6 |
| 48042 | CD4 | CD4 | CD4 antigen (p55) | 0.9 | 1.0 | 1.0 | 1.0 | 0.6 | 1.3 | 0.7 | 1.0 | 0.7 | 1.1 | 4.4 |
| 48166 | CDH15 | | | 0.8 | 1.2 | 1.5 | 1.3 | 1.2 | 1.0 | 1.3 | 1.1 | 1.1 | 1.0 | 4.4 |
| 50474 | CEBPA | C/EBP alpha | CCAAT/en-hancer | 0.9 | 1.0 | 1.5 | 1.4 | 1.1 | 1.0 | [symbol] | 1.5 | 1.0 | 1.0 | 4.9 |
| | | | binding protein | | | | | | | | | | | |
| | | | (C/EBP), alpha | | | | | | | | | | | |
| 46849 | CEBPD | CEBP delta | CCAAT/en-hancer | 5.8 | 4.5 | 0.8 | 1.0 | 0.8 | 1.1 | 1.1 | 1.0 | 0.9 | 1.0 | 4.1 |
| | | | binding protein | | | | | | | | | | | |
| | | | (C/EBP), delta | | | | | | | | | | | |
| 47891 | CEBPD | CEBP delta | CCAAT/en-hancer | 4.5 | 3.4 | 1.0 | 1.0 | 0.9 | 1.1 | 1.1 | 1.0 | 0.9 | 1.0 | 4.1 |
| | | | binding protein | | | | | | | | | | | |
| | | | (C/EBP), delta | | | | | | | | | | | |
| 47135 | CHKL | | | 1.2 | 1.0 | 0.7 | 1.0 | 0.7 | 1.3 | 0.8 | 1.0 | 0.7 | 1.2 | 4.6 |
| 47032 | [symbol] | CIT | citron (rho- | 0.7 | 1.1 | 0.6 | 1.5 | 0.8 | 1.1 | [symbol] | 1.4 | 0.8 | 1.2 | 5.2 |
| | | | interacting, | | | | | | | | | | | |
| | | | serine/threo-nine | | | | | | | | | | | |
| | | | kinase 2 | | | | | | | | | | | |
| 48972 | [symbol] | CIT | citron (rho- | 0.7 | 1.1 | 0.4 | 2.1 | 0.8 | 1.1 | 1.0 | 1.0 | 1.0 | 1.0 | 5.2 |
| | | | interacting, | | | | | | | | | | | |
| | | | serine/threo-nine | | | | | | | | | | | |
| | | | kinase 2 | | | | | | | | | | | |
| 49854 | COCH | | | 24.0 | 23.8 | 1.1 | 1.0 | 1.0 | 1.0 | 1.1 | 1.0 | 1.0 | 1.0 | 4.0 |
| 47563 | COL11A1 | COL11A1 | collagen, type XI, | 0.7 | 1.2 | 0.7 | 1.4 | 0.9 | 1.0 | 0.8 | 1.1 | 0.8 | 1.0 | 4.6 |
| | | | alpha 1 | | | | | | | | | | | |
| 50087 | COL4A1 | COL4A1 | collagen, type IV, | 2.9 | 2.7 | 1.4 | 1.1 | 1.0 | 1.0 | 1.2 | 1.2 | 1.1 | 1.0 | 4.3 |
| | | | alpha 1 | | | | | | | | | | | |
| 48068 | COL4A1 | COL4A1 | collagen, type IV, | 2.4 | 1.9 | 1.1 | 1.0 | 0.9 | 1.0 | 1.1 | 1.0 | 1.0 | 1.0 | 4.0 |
| | | | alpha 1 | | | | | | | | | | | |
| 49315 | COL4A2 | COL4A2 | collagen, type IV, | 1.9 | 1.6 | 1.5 | 1.3 | 1.2 | 1.0 | [symbol] | 1.3 | 1.1 | 1.0 | 4.6 |
| | | | alpha 2 | | | | | | | | | | | |
| 47197 | COL4A2 | COL4A2 | collagen, type IV, | 1.9 | 1.7 | 1.0 | 1.0 | 1.2 | 1.0 | 1.0 | 1.0 | 1.1 | 1.0 | 4.0 |
| | | | alpha 2 | | | | | | | | | | | |
| 49762 | COL4A4 | COL4A4 | collagen, type IV, | 0.7 | 1.0 | 1.2 | 1.1 | 1.0 | 1.0 | [symbol] | 1.4 | 1.1 | 1.0 | 4.5 |
| | | | alpha 4 | | | | | | | | | | | |
| 50705 | COL7A1 | COL7A1 | collagen, type VII, | 2.0 | 1.6 | 0.8 | 1.0 | 0.8 | 1.1 | 0.9 | 1.0 | 1.1 | 1.0 | 4.1 |
| | | | alpha 1 | | | | | | | | | | | |
| | | | (epidermolysis | | | | | | | | | | | |
| | | | bullosa | | | | | | | | | | | |
| 50564 | [symbol] | | | 0.6 | 1.5 | 0.6 | 1.4 | 0.7 | 1.3 | [symbol] | 1.3 | 0.7 | 1.2 | 5.1 |
| 49572 | CRMP1 | | | 0.7 | 1.3 | 1.4 | 1.1 | 1.1 | 1.0 | [symbol] | 1.5 | 1.3 | 1.2 | 4.7 |
| 49149 | CSF1 | M-CSF, | colony stimulating | 3.5 | 2.0 | 0.9 | 1.0 | 1.1 | 1.0 | 0.8 | 1.0 | 1.1 | 1.0 | 4.0 |
| | | macro-phage | factor 1 | | | | | | | | | | | |
| | | colony | (macrophage) | | | | | | | | | | | |
| | | stimulating | | | | | | | | | | | | |
| | | factor | | | | | | | | | | | | |
| 49278 | CSF1 | M-CSF, | colony stimulating | 3.5 | 2.0 | 0.9 | 1.0 | 1.1 | 1.0 | 0.8 | 1.0 | 1.1 | 1.0 | 4.0 |
| | | macro-phage | factor 1 | | | | | | | | | | | |
| | | colony | (macrophage) | | | | | | | | | | | |
| | | stimulating | | | | | | | | | | | | |
| | | factor | | | | | | | | | | | | |
| 48596 | CSF2 | G-CSF, | colony stimulating | 5.9 | 5.4 | 0.9 | 1.0 | 1.0 | 1.0 | 1.2 | 1.1 | 1.0 | 1.0 | 4.1 |
| | | granulocyte | factor 2 | | | | | | | | | | | |
| | | colony | (granulocyte- | | | | | | | | | | | |
| | | stimulating | macrophage | | | | | | | | | | | |
| | | factor | | | | | | | | | | | | |
| 48052 | CSF2 | G-CSF, | colony stimulating | 4.2 | 2.3 | 1.3 | 1.0 | 1.0 | 1.0 | 1.1 | 1.0 | 1.0 | 1.0 | 4.0 |
| | | granulocyte | factor 2 | | | | | | | | | | | |
| | | colony | (granulocyte- | | | | | | | | | | | |
| | | stimulating | macrophag | | | | | | | | | | | |
| | | factor | | | | | | | | | | | | |
| 49266 | CSF3 | GM-CSF, | colony stimulating | 2.1 | 1.6 | 1.0 | 1.0 | 1.0 | 1.0 | 0.9 | 1.0 | 1.0 | 1.0 | 4.0 |
| | | granulocyte- | factor 3 | | | | | | | | | | | |
| | | macrophage | (granulocyte) | | | | | | | | | | | |
| | | colony | | | | | | | | | | | | |
| | | stimulating | | | | | | | | | | | | |
| | | factor | | | | | | | | | | | | |
| 49137 | CSF3 | GM-CSF, | colony stimulating | 2.1 | 1.8 | 1.0 | 1.0 | 1.0 | 1.0 | 1.0 | 1.0 | 1.0 | 1.0 | 4.0 |
| | | granulocyte- | factor 3 | | | | | | | | | | | |
| | | macrophage | (granulocyte) | | | | | | | | | | | |
| | | colony | | | | | | | | | | | | |
| | | stimulating | | | | | | | | | | | | |
| | | factor | | | | | | | | | | | | |
| 48943 | CSF3R | G-CSF | colony stimulating | 0.7 | 1.0 | 0.5 | 1.7 | 1.1 | 1.0 | 1.0 | 1.0 | 1.1 | 1.0 | 4.7 |
| | | receptor | factor 3 receptor | | | | | | | | | | | |
| | | | (granulocyte) | | | | | | | | | | | |
| 48263 | CSPG2 | CSPG2 | chondroitin sulfate | 0.6 | 1.5 | 1.0 | 1.0 | 1.0 | 1.0 | 1.0 | 1.0 | 1.0 | 1.0 | 4.0 |
| | | | proteoglycan 2 | | | | | | | | | | | |
| | | | (versican) | | | | | | | | | | | |
| 50282 | CSRP1 | | | 2.5 | 1.7 | 1.6 | 1.3 | 1.4 | 1.3 | 1.4 | 1.2 | 1.2 | 1.0 | 4.8 |
| 49060 | CTGF | CTGF | connective tissue | 0.3 | 2.3 | 0.8 | 1.0 | 1.1 | 1.0 | [symbol] | 1.6 | 0.9 | 1.0 | 4.6 |
| | | | growth factor | | | | | | | | | | | |
| 48000 | CTSH | | | 0.7 | 1.3 | 1.4 | 1.3 | 1.1 | 1.0 | [symbol] | 1.4 | 1.2 | 1.0 | 4.7 |
| 47834 | CYP1A1 | | | 1.0 | 1.0 | 1.5 | 1.4 | 1.2 | 1.2 | 1.3 | 1.2 | 1.2 | 1.0 | 4.8 |
| 48721 | [symbol] | CD55 | decay | 4.0 | 2.7 | 2.1 | 2.0 | 1.0 | 1.0 | [symbol] | 1.6 | 1.1 | 1.0 | 5.6 |
| | | | accelerating factor | | | | | | | | | | | |
| | | | for complement | | | | | | | | | | | |
| | | | (CD55, Cr | | | | | | | | | | | |
| 46942 | [symbol] | CD55 | decay | 4.0 | 3.6 | 2.0 | 1.9 | 1.0 | 1.0 | [symbol] | 1.4 | 1.2 | 1.0 | 5.3 |
| | | | accelerating factor | | | | | | | | | | | |
| | | | for complement | | | | | | | | | | | |
| | | | (CD55, Cr | | | | | | | | | | | |
| 47976 | DDB2 | | | 0.6 | 1.5 | 0.7 | 1.3 | 0.9 | 1.0 | [symbol] | 1.5 | 1.0 | 1.0 | 4.8 |
| 49903 | DDXL | | | 1.0 | 1.0 | 0.7 | 1.4 | 1.0 | 1.0 | [symbol] | 1.3 | 0.9 | 1.0 | 4.7 |
| 49922 | DHCR24 | | | 0.9 | 1.1 | 1.4 | 1.2 | 1.0 | 1.0 | [symbol] | 1.5 | 1.0 | 1.0 | 4.7 |
| 47523 | DNAJB1 | DNAJB1 | DnaJ (Hsp40) | 1.0 | 1.0 | 1.0 | 1.0 | 0.9 | 1.0 | [symbol] | 1.4 | 1.2 | 1.1 | 4.5 |
| | | | homolog, | | | | | | | | | | | |
| | | | subfamily B, | | | | | | | | | | | |
| | | | member 1 | | | | | | | | | | | |
| 50745 | DNAJB1 | DNAJB1 | DnaJ (Hsp40) | 0.9 | 1.0 | 1.0 | 1.0 | 1.0 | 1.0 | [symbol] | 1.4 | 1.1 | 1.0 | 4.5 |
| | | | homolog, | | | | | | | | | | | |
| | | | subfamily B, | | | | | | | | | | | |
| | | | member 1 | | | | | | | | | | | |
| 49389 | DUSP1 | MKP-1, | dual specificity | 1.4 | 1.0 | 1.5 | 1.5 | 1.1 | 1.0 | 1.1 | 1.0 | 1.0 | 1.0 | 4.5 |
| | | PTPN10 | phosphatase 1 | | | | | | | | | | | |
| 50362 | DUSP10 | DUSP10 | dual specificity | 2.9 | 2.6 | 1.3 | 1.1 | 1.0 | 1.0 | 1.3 | 1.0 | 1.0 | 1.0 | 4.2 |
| | | | phosphatase 10 | | | | | | | | | | | |
| 46670 | DUSP5 | DUSP5 | dual specificity | 2.0 | 2.0 | 1.4 | 1.3 | 1.2 | 1.1 | 1.3 | 1.2 | 1.2 | 1.1 | 4.8 |
| | | | phosphatase 5 | | | | | | | | | | | |
| 49777 | DUSP6 | DUSP6 | dual specificity | 2.1 | 2.0 | 1.5 | 1.3 | 1.0 | 1.0 | 1.5 | 1.0 | 0.8 | 1.1 | 4.4 |
| | | | phosphatase 6 | | | | | | | | | | | |
| 47277 | DUSP6 | DUSP6 | dual specificity | 2.8 | 2.3 | 1.2 | 1.0 | 1.0 | 1.0 | 1.4 | 1.0 | 0.9 | 1.1 | 4.1 |
| | | | phosphatase 6 | | | | | | | | | | | |
| 49810 | DUSP6 | DUSP6 | dual specificity | 2.1 | 2.0 | 1.5 | 1.2 | 0.9 | 1.0 | 1.5 | 1.0 | 0.9 | 1.0 | 4.2 |
| | | | phosphatase 6 | | | | | | | | | | | |
| 50140 | [symbol] | ECT2 | epithelial cell | 1.2 | 1.1 | 0.5 | 1.6 | 0.9 | 1.1 | [symbol] | 1.7 | 0.8 | 1.2 | 5.6 |
| | | | transforming | | | | | | | | | | | |
| | | | sequence 2 | | | | | | | | | | | |
| | | | oncogene | | | | | | | | | | | |
| 48032 | [symbol] | ECT2 | epithelial cell | 1.1 | 1.0 | 0.5 | 1.4 | 0.8 | 1.1 | [symbol] | 1.7 | 0.8 | 1.2 | 5.4 |
| | | | transforming | | | | | | | | | | | |
| | | | sequence 2 | | | | | | | | | | | |
| | | | oncogene | | | | | | | | | | | |
| 47654 | EFEMP1 | EFEMP1 | EGF-containing | 0.8 | 1.1 | 1.1 | 1.0 | 1.1 | 1.0 | [symbol] 4 | 1.3 | 1.2 | 1.0 | 4.4 |
| | | | fibulin-like | | | | | | | | | | | |
| | | | extracellular | | | | | | | | | | | |
| | | | matrix p | | | | | | | | | | | |
| 49636 | EGFR | EGFR, ErbB1 | epidermal growth | 3.2 | 3.0 | 1.4 | 1.2 | 1.1 | 1.0 | 1.2 | 1.0 | 1.1 | 1.0 | 4.3 |
| | | | factor receptor | | | | | | | | | | | |
| | | | (avian erythrobla | | | | | | | | | | | |
| 49764 | [symbol] | EHF | ets homologous | 1.2 | 1.2 | 0.6 | 1.4 | 0.8 | 1.0 | [symbol] | 1.6 | 0.9 | 1.0 | 5.1 |
| | | | factor | | | | | | | | | | | |
| 47708 | EHF | EHF | ets homologous | 1.0 | 1.0 | 1.0 | 1.0 | 1.0 | 1.0 | [symbol] | 1.7 | 1.0 | 1.0 | 4.7 |
| | | | factor | | | | | | | | | | | |
| 46821 | ELA2 | elastase-2 | elastase 2, | 1.0 | 1.0 | 0.8 | 1.0 | 0.6 | 1.4 | 0.8 | 1.0 | 0.8 | 1.0 | 4.4 |
| | | | neutrophil | | | | | | | | | | | |
| 46813 | ELK3 | ELK3 | ELK3, ETS- | 1.9 | 1.6 | 1.0 | 1.0 | 1.0 | 1.0 | 0.9 | 1.1 | 1.1 | 1.0 | 4.1 |
| | | | domain protein | | | | | | | | | | | |
| | | | (SRF accessory | | | | | | | | | | | |
| | | | protein 2) | | | | | | | | | | | |
| 46955 | EREG | epiregulin | epiregulin | 5.4 | 3.3 | 1.2 | 1.0 | 1.4 | 1.0 | [symbol] | 1.3 | 1.2 | 1.1 | 4.4 |
| 50126 | EREG | epiregulin | epiregulin | 3.8 | 3.6 | 1.3 | 1.1 | 1.4 | 1.2 | [symbol] | 1.5 | 1.1 | 1.0 | 4.8 |
| 46988 | EREG | epiregulin | epiregulin | 5.4 | 3.4 | 1.1 | 1.1 | 1.3 | 1.0 | [symbol] | 1.5 | 1.1 | 1.0 | 4.6 |
| 47907 | [symbol] | ERG | v-ets avian | 4.7 | 4.1 | 1.0 | 1.0 | 1.0 | 1.0 | 1.3 | 1.2 | 2.2 | [symbol] | 5.3 |
| | | | erythroblastosis | | | | | | | | | | | |
| | | | virus E26 | | | | | | | | | | | |
| | | | oncogene re | | | | | | | | | | | |
| 47554 | ERG | ERG | v-ets avian | 1.0 | 1.0 | 1.0 | 1.0 | 0.9 | 1.0 | [symbol] | 1.3 | 1.9 | 1.4 | 4.7 |
| | | | erythroblastosis | | | | | | | | | | | |
| | | | virus E26 | | | | | | | | | | | |
| | | | oncogene re | | | | | | | | | | | |
| 50339 | ETV6 | ETV6 | ets variant gene 6 | 2.5 | 2.1 | 1.3 | 1.1 | 1.0 | 1.0 | 1.2 | 1.2 | 1.1 | 1.0 | 4.4 |
| | | | (TEL oncogene) | | | | | | | | | | | |
| 49695 | ETV6 | ETV6 | ets variant gene 6 | 2.2 | 2.1 | 1.0 | 1.0 | 1.0 | 1.0 | 1.2 | 1.2 | 1.0 | 1.0 | 4.2 |
| | | | (TEL oncogene) | | | | | | | | | | | |
| 47539 | F2 | F2 | coagulation factor | 5.6 | 3.7 | 1.2 | 1.1 | 1.1 | 1.1 | 1.0 | 1.0 | 1.3 | 1.1 | 4.3 |
| | | | II (thrombin) | | | | | | | | | | | |
| 48998 | [symbol] | | | 0.8 | 1.0 | 2.1 | 2.0 | 1.1 | 1.1 | [symbol] | 2.0 | 1.0 | 1.0 | 6.0 |
| 49529 | FGA | | | 0.4 | 2.1 | 1.5 | 1.3 | 0.9 | 1.0 | [symbol] | 1.3 | 0.8 | 1.1 | 4.7 |
| 46993 | FOSL1 | FOSL1, Fra1 | FOS-like antigen-1 | 2.8 | 2.0 | 1.1 | 1.0 | 1.2 | 1.0 | 0.9 | 1.0 | 1.1 | 1.0 | 4.0 |
| 50475 | FOSL1 | FOSL1, Fra1 | FOS-like antigen-1 | 2.7 | 1.9 | 1.1 | 1.0 | 1.1 | 1.0 | 1.0 | 1.0 | 1.2 | 1.0 | 4.1 |
| 50508 | FOSL1 | FOSL1, Fra1 | FOS-like antigen-1 | 2.7 | 2.0 | 1.0 | 1.0 | 1.1 | 1.0 | 1.0 | 1.0 | 1.1 | 1.0 | 4.0 |
| 48969 | FOSL2 | FOSL2, Fra2 | FOS-like antigen 2 | 2.3 | 2.0 | 1.3 | 1.2 | 1.0 | 1.0 | [symbol] 4 | 1.3 | 1.1 | 1.0 | 4.5 |
| 48270 | FOSL2 | FOSL2, Fra2 | FOS-like antigen 2 | 2.3 | 2.2 | 1.2 | 1.1 | 1.0 | 1.0 | [symbol] | 1.3 | 1.1 | 1.0 | 4.4 |
| 49798 | FRAG1 | FRAG1 | FGF receptor | 0.7 | 1.4 | 1.2 | 1.1 | 1.1 | 1.1 | [symbol] 4 | 1.4 | 1.2 | 1.2 | 4.7 |
| | | | activating protein 1 | | | | | | | | | | | |
| 49793 | FRAG1 | FRAG1 | FGF receptor | 0.7 | 1.3 | 1.2 | 1.0 | 1.1 | 1.1 | [symbol] 4 | 1.3 | 1.1 | 1.1 | 4.5 |
| | | | activating protein 1 | | | | | | | | | | | |
| 49765 | FRAG1 | FRAG1 | FGF receptor | 0.6 | 1.5 | 1.2 | 1.1 | 1.1 | 1.0 | 1.3 | 1.2 | 1.1 | 1.1 | 4.4 |
| | | | activating protein 1 | | | | | | | | | | | |
| 48137 | FRDA | | | 2.3 | 2.0 | 0.8 | 1.1 | 1.0 | 1.0 | 0.7 | 1.1 | 0.8 | 1.0 | 4.2 |
| 49862 | FUT8 | | | 0.7 | 1.4 | 1.4 | 1.2 | 1.0 | 1.0 | [symbol] 4 | 1.4 | 1.0 | 1.0 | 4.5 |
| 49883 | FVT1 | | | 1.1 | 1.0 | 1.4 | 1.2 | 1.1 | 1.0 | [symbol] 4 | 1.3 | 1.0 | 1.0 | 4.6 |
| 48615 | GADD45B | MyD118 | growth arrest and | 0.4 | 2.1 | 1.2 | 1.0 | 0.6 | 1.3 | [symbol] | 1.3 | 1.0 | 1.0 | 4.6 |
| | | | DNA-damage- | | | | | | | | | | | |
| | | | inducible, beta | | | | | | | | | | | |
| 50142 | GBP1 | IFN induced | guanylate binding | 10.3 | 6.1 | 1.0 | 1.0 | 1.0 | 1.0 | 0.7 | 1.2 | 1.1 | 1.0 | 4.2 |
| | | GBP-1 | protein 1, | | | | | | | | | | | |
| | | | interferon- | | | | | | | | | | | |
| | | | inducible, | | | | | | | | | | | |
| 49059 | GCH1 | GTP | GTP | 6.9 | 6.1 | 1.2 | 1.1 | 0.9 | 1.0 | 1.3 | 1.0 | 0.9 | 1.0 | 4.2 |
| | | cyclohydrolase I | cyclohydrolase 1 | | | | | | | | | | | |
| | | | (dopa-responsive | | | | | | | | | | | |
| | | | dystonia) | | | | | | | | | | | |
| 49092 | GCH1 | GTP | GTP | 6.8 | 6.3 | 1.4 | 1.3 | 0.9 | 1.0 | 1.2 | 1.0 | 0.9 | 1.0 | 4.3 |
| | | cyclohydrolase I | cyclohydrolase 1 | | | | | | | | | | | |
| | | | (dopa-responsive | | | | | | | | | | | |
| | | | dystonia) | | | | | | | | | | | |
| 46977 | GCH1 | GTP | GTP | 8.1 | 4.9 | 1.2 | 1.0 | 0.9 | 1.0 | 1.2 | 1.0 | 1.0 | 1.0 | 4.0 |
| | | cyclohydrolase I | cyclohydrolase 1 | | | | | | | | | | | |
| | | | (dopa-responsive | | | | | | | | | | | |
| | | | dystonia) | | | | | | | | | | | |
| 50653 | GM2A | | | 1.0 | 1.0 | 1.2 | 1.1 | 1.1 | 1.0 | [symbol] | 1.4 | 1.0 | 1.0 | 4.4 |
| 50689 | GNB5 | G protein, beta 5 | guanine | 0.9 | 1.0 | 1.3 | 1.2 | 1.1 | 1.0 | [symbol] | 1.3 | 1.2 | 1.0 | 4.5 |
| | | | nucleotide binding | | | | | | | | | | | |
| | | | protein (G | | | | | | | | | | | |
| | | | protein), be | | | | | | | | | | | |
| 50671 | GNG3 | G protein, | guanine | 0.9 | 1.0 | 1.6 | 1.3 | 1.0 | 1.0 | 0.8 | 1.0 | 1.0 | 1.0 | 4.3 |
| | | gamma 3 | nucleotide binding | | | | | | | | | | | |
| | | | protein (G | | | | | | | | | | | |
| | | | protein), ga | | | | | | | | | | | |
| 48713 | GPRK7 | MNK2 | G protein-coupled | 0.6 | 1.4 | 1.7 | 1.4 | 1.1 | 1.0 | 1.3 | 1.2 | 1.1 | 1.0 | 4.6 |
| | | | receptor kinase 7 | | | | | | | | | | | |
| 46825 | GPRK7 | MNK2 | G protein-coupled | 0.7 | 1.1 | 1.5 | 1.4 | 1.1 | 1.0 | 1.2 | 1.1 | 1.1 | 1.0 | 4.5 |
| | | | receptor kinase 7 | | | | | | | | | | | |
| 48548 | GRAP2 | GRAP2 | GRB2-related | 1.2 | 1.0 | 1.0 | 1.0 | 0.7 | 1.4 | 0.8 | 1.1 | 1.0 | 1.0 | 4.5 |
| | | | adaptor protein 2 | | | | | | | | | | | |
| 46936 | GRB10 | GRB10 | growth factor | 1.4 | 1.2 | 1.6 | 1.3 | 1.1 | 1.1 | 1.3 | 1.0 | 1.1 | 1.0 | 4.4 |
| | | | receptor-bound | | | | | | | | | | | |
| | | | protein 10 | | | | | | | | | | | |
| 46962 | GRB10 | GRB10 | growth factor | 1.5 | 1.4 | 1.6 | 1.5 | 1.1 | 1.0 | 1.2 | 1.0 | 1.1 | 1.0 | 4.5 |
| | | | receptor-bound | | | | | | | | | | | |
| | | | protein 10 | | | | | | | | | | | |
| 49586 | GRIN2C | | | 39.1 | 25.1 | 1.2 | 1.1 | 0.9 | 1.0 | 1.0 | 1.0 | 0.9 | 1.0 | 4.1 |
| 49754 | GRO1 | GRO1 | GRO1 oncogene | 65.9 | 48.3 | 1.4 | 1.2 | 0.9 | 1.0 | 1.1 | 1.1 | 1.0 | 1.0 | 4.3 |
| | | | (melanoma growth | | | | | | | | | | | |
| | | | stimulating activit | | | | | | | | | | | |
| 50462 | GRO1 | GRO1 | GRO1 oncogene | 83.9 | 45.6 | 1.5 | 1.3 | 1.0 | 1.0 | 1.1 | 1.0 | 1.0 | 1.0 | 4.3 |
| | | | (melanoma growth | | | | | | | | | | | |
| | | | stimulating activit | | | | | | | | | | | |
| 47632 | GRO2 | GRO2 | GRO2 oncogene | 68.5 | 50.8 | 1.5 | 1.0 | 1.0 | 1.0 | 1.0 | 1.0 | 1.0 | 1.0 | 4.0 |
| 47558 | GRO2 | GRO2 | GRO2 oncogene | 28.9 | 21.2 | 1.0 | 1.0 | 1.0 | 1.0 | 1.0 | 1.0 | 1.0 | 1.0 | 4.0 |
| 49692 | GRO3 | GRO3 | GRO3 oncogene | 97.5 | 64.4 | 1.6 | 1.2 | 1.0 | 1.0 | 1.1 | 1.0 | 1.0 | 1.0 | 4.3 |
| 48330 | GRO3 | GRO3 | GRO3 oncogene | 79.2 | 47.5 | 1.6 | 1.2 | 0.9 | 1.0 | 1.1 | 1.0 | 1.1 | 1.0 | 4.2 |
| 49688 | GSK3B | GSK3B | glycogen synthase | 1.5 | 1.4 | 1.4 | 1.4 | 1.0 | 1.0 | [symbol] 4 | 1.3 | 0.9 | 1.0 | 4.7 |
| | | | kinase 3 beta | | | | | | | | | | | |
| 49032 | GSK3B | GSK3B | glycogen synthase | 1.4 | 1.2 | 1.4 | 1.3 | 1.0 | 1.0 | 1.4 | 1.2 | 1.1 | 1.0 | 4.5 |
| | | | kinase 3 beta | | | | | | | | | | | |
| 47492 | HDGF | | | 2.3 | 2.0 | 0.8 | 1.1 | 1.0 | 1.0 | 0.9 | 1.0 | 1.0 | 1.0 | 4.1 |
| 47831 | HEC | | | 1.1 | 1.0 | 0.6 | 1.4 | 0.9 | 1.0 | [symbol] | 1.3 | 1.0 | 1.0 | 4.8 |
| 46778 | HMG2 | | | 1.1 | 1.0 | 0.6 | 1.1 | 0.8 | 1.1 | [symbol] | 1.4 | 0.8 | 1.0 | 4.6 |
| 49347 | [symbol] | | | 0.9 | 1.0 | 0.5 | 1.6 | 1.0 | 1.0 | [symbol] | 1.5 | 1.0 | 1.0 | 5.0 |
| 47922 | HS3ST3A1 | HS3ST3A1 | heparan sulfate | 1.0 | 1.0 | 1.0 | 1.0 | 1.0 | 1.0 | [symbol] | 1.5 | 1.2 | 1.1 | 4.6 |
| | | | (glucosamine) 3- | | | | | | | | | | | |
| | | | O-sulfotransferase | | | | | | | | | | | |
| 49113 | HS3ST3A1 | HS3ST3A1 | heparan sulfate | 2.9 | 2.4 | 1.4 | 1.2 | 1.0 | 1.0 | [symbol] | 1.5 | 1.1 | 1.1 | 4.8 |
| | | | (glucosamine) 3- | | | | | | | | | | | |
| | | | O-sulfotransferase | | | | | | | | | | | |
| 47414 | HSF4 | | | 0.7 | 1.3 | 0.7 | 1.1 | 0.7 | 1.3 | 0.8 | 1.1 | 1.0 | 1.0 | 4.4 |
| 49417 | [symbol] | HSPA1B | heat shock 70 kD | 1.0 | 1.0 | 1.5 | 1.3 | 1.0 | 1.0 | [symbol] | 2.0 | [symbol] | 1.3 | 5.6 |
| | | | protein 1B | | | | | | | | | | | |
| 49064 | [symbol] | HSPA1B | heat shock 70 kD | 1.0 | 1.0 | 1.5 | 1.3 | 1.0 | 1.0 | [symbol] | 1.9 | [symbol] | 1.3 | 5.6 |
| | | | protein 1B | | | | | | | | | | | |
| 48618 | [symbol] | HSPA1B | heat shock 70 kD | 1.1 | 1.0 | 1.5 | 1.3 | 1.0 | 1.0 | [symbol] | 1.9 | [symbol] | 1.3 | 5.5 |
| | | | protein 1B | | | | | | | | | | | |
| 47185 | [symbol] | HSPA1B | heat shock 70 kD | 1.2 | 1.0 | 1.3 | 1.1 | 1.0 | 1.0 | [symbol] | 1.9 | 1.4 | 1.2 | 5.3 |
| | | | protein 1B | | | | | | | | | | | |
| 49516 | HSPA5 | HSPA5 | heat shock 70 kD | 1.1 | 1.0 | 1.6 | 1.3 | 1.2 | 1.1 | 1.2 | 1.1 | 1.2 | 1.0 | 4.6 |
| | | | protein 5 (glucose- | | | | | | | | | | | |
| | | | regulated prote | | | | | | | | | | | |
| 48094 | HSPA6 | HSPA6 | heat shock 70 kD | 1.0 | 1.0 | 1.1 | 1.0 | 1.0 | 1.0 | [symbol] | 1.6 | 1.2 | 1.0 | 4.7 |
| | | | protein 6 | | | | | | | | | | | |
| | | | (HSP70B′) | | | | | | | | | | | |
| 49436 | HSPB1 | HSPB1 | heat shock 27 kD | 0.6 | 1.5 | 1.0 | 1.0 | 1.2 | 1.1 | 1.3 | 1.0 | 1.2 | 1.0 | 4.1 |
| | | | protein 1 | | | | | | | | | | | |
| 48550 | HSPB2 | HSPB2 | heat shock 27 kD | 2.5 | 2.1 | 1.0 | 1.0 | 1.0 | 1.0 | 1.1 | 1.0 | 1.0 | 1.0 | 4.1 |
| | | | protein 2 | | | | | | | | | | | |
| 48421 | HSPB2 | HSPB2 | heat shock 27 kD | 2.6 | 2.4 | 1.2 | 1.0 | 0.9 | 1.0 | 1.0 | 1.0 | 1.1 | 1.0 | 4.0 |
| | | | protein 2 | | | | | | | | | | | |
| 48515 | HYA22 | | | 0.6 | 1.6 | 1.0 | 1.0 | 1.0 | 1.0 | 1.1 | 1.0 | 1.3 | 1.2 | 4.2 |
| 49804 | ICAM1 | ICAM1 | intercellular | 46.2 | 31.4 | 1.2 | 1.1 | 0.9 | 1.0 | 0.8 | 1.1 | 1.0 | 1.0 | 4.2 |
| | | | adhesion | | | | | | | | | | | |
| | | | molecule 1 | | | | | | | | | | | |
| | | | (CD54), human rh | | | | | | | | | | | |
| 49771 | ICAM1 | ICAM1 | intercellular | 49.6 | 33.2 | 1.2 | 1.1 | 0.9 | 1.0 | 0.8 | 1.0 | 1.1 | 1.0 | 4.1 |
| | | | adhesion | | | | | | | | | | | |
| | | | molecule 1 | | | | | | | | | | | |
| | | | (CD54), human rh | | | | | | | | | | | |
| 49140 | ICAM1 | ICAM1 | intercellular | 16.2 | 12.4 | 1.1 | 1.0 | 1.0 | 1.0 | 0.8 | 1.0 | 1.1 | 1.0 | 4.0 |
| | | | adhesion | | | | | | | | | | | |
| | | | molecule 1 | | | | | | | | | | | |
| | | | (CD54), human rh | | | | | | | | | | | |
| 50084 | [symbol] | | | 0.7 | 1.0 | 1.7 | 1.6 | 0.8 | 1.1 | [symbol] | 1.4 | 0.9 | 1.1 | 5.2 |
| 49222 | IFNAR1 | IFN alpha, | interferon (alpha, | 1.9 | 1.8 | 1.3 | 1.2 | 1.0 | 1.0 | 1.1 | 1.1 | 1.1 | 1.0 | 4.3 |
| | | beta, omega | beta and omega) | | | | | | | | | | | |
| | | receptor 1 | receptor 1 | | | | | | | | | | | |
| 49265 | IFNAR2 | IFN alpha, | interferon (alpha, | 3.0 | 2.5 | 1.2 | 1.2 | 1.0 | 1.0 | 1.1 | 1.0 | 1.0 | 1.0 | 4.2 |
| | | beta, omega | beta and omega) | | | | | | | | | | | |
| | | receptor 2 | receptor 2 | | | | | | | | | | | |
| 48913 | IFNAR2 | IFN alpha, | interferon (alpha, | 3.0 | 2.7 | 1.2 | 1.2 | 1.0 | 1.0 | 1.1 | 1.0 | 1.0 | 1.0 | 4.2 |
| | | beta, omega | beta and omega) | | | | | | | | | | | |
| | | receptor 2 | receptor 2 | | | | | | | | | | | |
| 47903 | IFNGR1 | IFN gamma | interferon gamma | 4.6 | 4.0 | 1.0 | 1.0 | 0.9 | 1.0 | 0.8 | 1.0 | 0.9 | 1.0 | 4.0 |
| | | receptor 1 | receptor 1 | | | | | | | | | | | |
| 50311 | IFNGR1 | IFN gamma | interferon gamma | 4.6 | 4.5 | 1.0 | 1.0 | 0.9 | 1.0 | 1.1 | 1.0 | 0.9 | 1.0 | 4.0 |
| | | receptor 1 | receptor 1 | | | | | | | | | | | |
| 47225 | IFNGR2 | IFN gamma | interferon gamma | 4.1 | 3.8 | 1.1 | 1.1 | 0.9 | 1.0 | 1.0 | 1.0 | 1.2 | 1.0 | 4.1 |
| | | receptor 2 | receptor 2 | | | | | | | | | | | |
| | | | (interferon gamma | | | | | | | | | | | |
| | | | tran | | | | | | | | | | | |
| 47582 | IFNGR2 | IFN gamma | interferon gamma | 3.9 | 3.7 | 1.0 | 1.0 | 1.0 | 1.0 | 1.0 | 1.0 | 1.0 | 1.0 | 4.0 |
| | | receptor 2 | receptor 2 | | | | | | | | | | | |
| | | | (interferon gamma | | | | | | | | | | | |
| | | | tran | | | | | | | | | | | |
| 48725 | IKKE | IKK-related K | IKK-related kinase | 4.1 | 3.2 | 1.0 | 1.0 | 0.9 | 1.0 | 0.9 | 1.0 | 0.9 | 1.1 | 4.1 |
| | | epsilon, IKK-I | epsilon; inducible | | | | | | | | | | | |
| | | | IkappaB kina | | | | | | | | | | | |
| 50342 | IKKE | IKK-related K | IKK-related kinase | 5.9 | 4.0 | 1.2 | 1.1 | 1.0 | 1.0 | 1.1 | 1.0 | 1.1 | 1.0 | 4.1 |
| | | epsilon, IKK-I | epsilon; inducible | | | | | | | | | | | |
| | | | IkappaB kina | | | | | | | | | | | |
| 48961 | IL10RB | IL-10R beta | interleukin 10 | 1.8 | 1.5 | 1.1 | 1.1 | 1.0 | 1.0 | 1.1 | 1.1 | 1.1 | 1.1 | 4.2 |
| | | | receptor, beta | | | | | | | | | | | |
| 50013 | IL13RA1 | IL-13Ralpha′ | interleukin 13 | 0.6 | 1.2 | 1.3 | 1.2 | 1.1 | 1.1 | [symbol] 4 | 1.3 | 1.0 | 1.0 | 4.6 |
| | | | receptor, alpha 1 | | | | | | | | | | | |
| 48423 | IL1A | IL-1, alpha | interleukin 1, | 1.9 | 1.8 | 1.1 | 1.0 | 1.0 | 1.0 | 1.1 | 1.0 | 1.0 | 1.0 | 4.0 |
| | | | alpha | | | | | | | | | | | |
| 48552 | IL1A | IL-1, alpha | interleukin 1, | 1.9 | 1.9 | 1.1 | 1.0 | 1.0 | 1.0 | 1.0 | 1.0 | 1.0 | 1.0 | 4.0 |
| | | | alpha | | | | | | | | | | | |
| 46970 | IL1B | IL-1, beta | interleukin 1, beta | 29.3 | 23.7 | 1.0 | 1.0 | 1.0 | 1.0 | 1.1 | 1.0 | [symbol] 4 | 1.3 | 4.3 |
| 47656 | IL1B | IL-1, beta | interleukin 1, beta | 15.9 | 14.8 | 1.0 | 1.0 | 1.0 | 1.0 | 1.2 | 1.0 | [symbol] 4 | 1.3 | 4.3 |
| 48637 | IL1R1 | IL-1RI | interleukin 1 | 0.6 | 1.6 | 1.1 | 1.0 | 0.8 | 1.2 | 1.4 | 1.1 | 0.9 | 1.0 | 4.3 |
| | | | receptor, type I | | | | | | | | | | | |
| 47204 | IL1RL1LG | T1/ST2 R | putative T1/ST2 | 0.9 | 1.0 | 0.7 | 1.4 | 0.9 | 1.0 | 0.9 | 1.0 | 1.0 | 1.0 | 4.4 |
| | | binding protein | receptor binding | | | | | | | | | | | |
| | | | protein | | | | | | | | | | | |
| 48324 | IL3RA | IL-3R alpha | interleukin 3 | 1.7 | 1.2 | 1.9 | 1.5 | 1.0 | 1.0 | 1.3 | 1.0 | 1.0 | 1.0 | 4.5 |
| | | | receptor, alpha | | | | | | | | | | | |
| | | | (low affinity) | | | | | | | | | | | |
| 46856 | IL8 | interleukin-8 | interleukin 8 | 239.5 | 161.1 | 2.2 | 1.9 | 0.8 | 1.0 | 1.0 | 1.0 | 1.1 | 1.0 | 4.9 |
| 49873 | [symbol] | | | 0.6 | 1.1 | 2.1 | 1.3 | 0.9 | 1.0 | [symbol] | 1.9 | 1.0 | 1.0 | 5.2 |
| 46642 | IRAK2 | IRAK-2 | interleukin-1 | 8.9 | 7.4 | 1.0 | 1.0 | 1.0 | 1.0 | 0.9 | 1.0 | 1.2 | 1.0 | 4.0 |
| | | | receptor- | | | | | | | | | | | |
| | | | associated kinase 2 | | | | | | | | | | | |
| 46618 | ISG15 | ISG15, IFN | interferon- | 5.6 | 4.1 | 1.3 | 1.0 | 1.5 | 1.0 | 1.2 | 1.2 | 1.6 | 1.0 | 4.2 |
| | | stimulated | stimulated protein, | | | | | | | | | | | |
| | | protein | 15 kDa | | | | | | | | | | | |
| 49054 | ITGA2 | integrin, alpha | integrin, alpha 2 | 1.7 | 1.3 | 1.5 | 1.3 | 1.0 | 1.0 | 1.3 | 1.2 | 1.1 | 1.0 | 4.6 |
| | | 2, CD49B, | (CD49B, alpha 2 | | | | | | | | | | | |
| | | VLA-2 | subunit of VLA-2 | | | | | | | | | | | |
| 46686 | ITGAV | integrin alpha | integrin, alpha V | 2.4 | 1.9 | 0.8 | 1.1 | 1.0 | 1.0 | 0.9 | 1.1 | 1.0 | 1.0 | 4.3 |
| | | V, CD51 | (vitronectin | | | | | | | | | | | |
| | | | receptor, alpha pol | | | | | | | | | | | |
| 50607 | ITGB3BP | | | 0.8 | 1.0 | 0.6 | 1.0 | 0.9 | 1.1 | [symbol] | 1.4 | 0.8 | 1.2 | 4.7 |
| 48228 | JAK1 | Jak1 | Janus kinase 1 (a | 1.7 | 1.5 | 1.1 | 1.0 | 1.0 | 1.0 | 1.0 | 1.0 | 1.0 | 1.0 | 4.0 |
| | | | protein tyrosine | | | | | | | | | | | |
| | | | kinase) | | | | | | | | | | | |
| 49322 | JUNB | JunB | jun B proto- | 1.2 | 1.1 | 1.7 | 1.6 | 0.9 | 1.0 | 1.3 | 1.0 | 0.8 | 1.1 | 4.7 |
| | | | oncogene | | | | | | | | | | | |
| 49319 | JUNB | JunB | jun B proto- | 1.2 | 1.1 | 1.6 | 1.4 | 1.0 | 1.0 | 1.3 | 1.0 | 0.9 | 1.0 | 4.4 |
| | | | oncogene | | | | | | | | | | | |
| 49167 | KDELR3 | | | 0.9 | 1.0 | 1.5 | 1.1 | 1.1 | 1.0 | [symbol] | 1.6 | 1.1 | 1.0 | 4.8 |
| 48663 | KIAA0089 | | | 0.6 | 1.3 | 1.5 | 1.3 | 1.2 | 1.1 | [symbol] | 1.3 | 1.0 | 1.0 | 4.7 |
| 46911 | KIAA0170 | | | 1.0 | 1.0 | 0.6 | 1.5 | 0.9 | 1.0 | [symbol] | 1.4 | 0.9 | 1.0 | 4.9 |
| 50052 | KIAA0171 | | | 1.9 | 1.8 | 0.9 | 1.0 | 0.9 | 1.0 | 0.9 | 1.1 | 0.9 | 1.0 | 4.2 |
| 47775 | KIAA0365 | | | 0.9 | 1.0 | 0.7 | 1.3 | 0.8 | 1.0 | 0.8 | 1.2 | 0.9 | 1.0 | 4.5 |
| 47944 | KIAA0395 | | | 0.9 | 1.0 | 0.9 | 1.0 | 1.1 | 1.0 | 1.0 | 1.0 | [symbol] | 1.4 | 4.4 |
| 47762 | KIAA0414 | | | 0.9 | 1.0 | 0.7 | 1.3 | 0.8 | 1.0 | 0.8 | 1.2 | 0.9 | 1.0 | 4.5 |
| 48520 | KIAA0418 | | | 1.2 | 1.0 | 1.4 | 1.3 | 1.0 | 1.0 | [symbol] | 1.5 | 1.2 | 1.0 | 4.8 |
| 48918 | KIAA0514 | | | 0.8 | 1.2 | 1.4 | 1.3 | 1.1 | 1.0 | [symbol] 4 | 1.3 | 1.1 | 1.0 | 4.7 |
| 48162 | KNSL2 | | | 0.9 | 1.0 | 0.7 | 1.2 | 0.9 | 1.0 | [symbol] | 1.3 | 1.0 | 1.0 | 4.5 |
| 50568 | KNSL5 | | | 1.1 | 1.0 | 0.5 | 1.2 | 0.8 | 1.1 | [symbol] | 0.6 | 0.8 | 1.1 | 4.8 |
| 48856 | KRT2A | | | 0.7 | 1.2 | 0.6 | 1.4 | 1.0 | 1.0 | 0.6 | 1.0 | 1.0 | 1.0 | 4.4 |
| 47437 | KRT4 | | | 0.8 | 1.1 | 0.7 | 1.4 | 1.0 | 1.0 | 0.8 | 1.0 | 1.2 | 1.1 | 4.4 |
| 50042 | LAMB3 | LAMB3 | laminin, beta 3 | 13.3 | 11.0 | 0.7 | 1.3 | 0.9 | 1.0 | [symbol] | 1.4 | 0.8 | 1.1 | 4.8 |
| | | | (nicein (125 kD), | | | | | | | | | | | |
| | | | kalinin (140 kD), | | | | | | | | | | | |
| 50037 | [symbol] | LAMC2 | laminin, gamma 2 | 1.0 | 1.0 | 2.0 | 1.5 | 1.0 | 1.0 | [symbol] 0 | 1.5 | 1.2 | 1.0 | 5.0 |
| | | | (nicein (100 kD), | | | | | | | | | | | |
| | | | kalinin (105 kD), | | | | | | | | | | | |
| 50393 | LAMC2 | LAMC2 | laminin, gamma 2 | 6.5 | 6.1 | 1.3 | 1.0 | 1.3 | 1.0 | [symbol] | 1.4 | 1.1 | 1.0 | 4.4 |
| | | | (nicein (100 kD), | | | | | | | | | | | |
| | | | kalinin (105 kD), | | | | | | | | | | | |
| 49163 | LGALS3 | | | 1.0 | 1.0 | 1.3 | 1.2 | 1.2 | 1.1 | [symbol] 4 | 1.3 | 1.2 | 1.1 | 4.7 |
| 50025 | LIFR | LIFR | leukemia inhibitory | 1.0 | 1.0 | 1.0 | 1.0 | 1.0 | 1.0 | 1.0 | 1.0 | [symbol] | 1.6 | 4.6 |
| | | | factor receptor | | | | | | | | | | | |
| 47191 | LIFR | LIFR | leukemia inhibitory | 1.0 | 1.0 | 1.4 | 1.3 | 1.1 | 1.0 | 1.1 | 1.0 | 1.2 | 1.2 | 4.5 |
| | | | factor receptor | | | | | | | | | | | |
| 49857 | LLGL2 | | | 0.6 | 1.6 | 1.0 | 1.0 | 1.0 | 1.0 | 1.2 | 1.0 | 1.0 | 1.0 | 4.0 |
| 50349 | LOC51295 | ECSIT | ECSIT | 0.9 | 1.1 | 0.6 | 1.4 | 1.1 | 1.0 | [symbol] | 1.4 | 1.1 | 1.0 | 4.8 |
| 46875 | [symbol] | | | 1.0 | 1.0 | 2.1 | 1.9 | 1.1 | 1.1 | 1.1 | 1.0 | 1.0 | 1.0 | 5.0 |
| 48673 | LRDD | LRDD | leucine rich repeat | 0.9 | 1.0 | 0.7 | 1.3 | 0.8 | 1.1 | [symbol] | 1.4 | 1.0 | 1.0 | 4.7 |
| | | | and death domain | | | | | | | | | | | |
| | | | containing pr | | | | | | | | | | | |
| 48748 | LTB | LT, beta | lymphotoxin beta | 1.0 | 1.0 | 1.0 | 1.0 | 1.0 | 1.0 | [symbol] | 1.3 | [symbol] | 1.3 | 4.6 |
| | | | (TNF superfamily, | | | | | | | | | | | |
| | | | member 3) | | | | | | | | | | | |
| 49292 | LTB | LT, beta | lymphotoxin beta | 4.6 | 2.7 | 1.0 | 1.0 | 1.0 | 1.0 | [symbol] | 1.6 | 1.3 | 1.0 | 4.7 |
| | | | (TNF superfamily, | | | | | | | | | | | |
| | | | member 3) | | | | | | | | | | | |
| 46676 | LYN | Lyn | v-yes-1 | 2.1 | 2.0 | 1.3 | 1.2 | 1.0 | 1.0 | 1.2 | 1.2 | 1.0 | 1.0 | 4.3 |
| | | | Yamaguchi | | | | | | | | | | | |
| | | | sarcoma viral | | | | | | | | | | | |
| | | | related oncogene h | | | | | | | | | | | |
| 46605 | LYN | Lyn | v-yes-1 | 2.5 | 2.2 | 1.3 | 1.1 | 1.0 | 1.0 | 1.2 | 1.1 | 1.0 | 1.0 | 4.2 |
| | | | Yamaguchi | | | | | | | | | | | |
| | | | sarcoma viral | | | | | | | | | | | |
| | | | related oncogene h | | | | | | | | | | | |
| 47158 | MAD | MAD | MAX dimerization | 2.0 | 1.8 | 1.3 | 1.1 | 0.9 | 1.0 | 1.0 | 1.0 | 1.1 | 1.0 | 4.1 |
| | | | protein | | | | | | | | | | | |
| 47029 | MAD | MAD | MAX dimerization | 1.9 | 1.6 | 1.1 | 1.0 | 0.9 | 1.0 | 1.1 | 1.0 | 1.1 | 1.0 | 4.0 |
| | | | protein | | | | | | | | | | | |
| 46776 | MAD2L1 | | | 0.9 | 1.0 | 0.7 | 1.1 | 0.8 | 1.1 | [symbol] | 1.4 | 0.9 | 1.0 | 4.6 |
| 47705 | MAD4 | MAD4 | Mad4 homolog | 1.0 | 1.0 | 1.5 | 1.1 | 1.0 | 1.0 | [symbol] 0 | 1.4 | 1.1 | 1.0 | 4.5 |
| 47087 | MADH3 | | | 2.3 | 1.9 | 0.8 | 1.1 | 1.0 | 1.0 | 0.9 | 1.1 | 1.0 | 1.0 | 4.3 |
| 49678 | MAP2K1 | MEK1, MKK1 | mitogen-activated | 2.3 | 2.2 | 1.2 | 1.1 | 1.0 | 1.0 | 1.2 | 1.0 | 0.9 | 1.0 | 4.1 |
| | | | protein kinase | | | | | | | | | | | |
| | | | kinase 1 | | | | | | | | | | | |
| 50482 | MAP2K2 | MAP2K2 | mitogen-activated | 2.2 | 2.1 | 1.0 | 1.0 | 1.1 | 1.0 | 0.9 | 1.0 | 0.9 | 1.0 | 4.0 |
| | | | protein kinase | | | | | | | | | | | |
| | | | kinase 2 | | | | | | | | | | | |
| 48028 | MAP2K3 | MKK3 | mitogen-activated | 3.0 | 2.7 | 1.0 | 1.0 | 1.0 | 1.0 | 1.1 | 1.0 | 1.1 | 1.1 | 4.1 |
| | | | protein kinase | | | | | | | | | | | |
| | | | kinase 3 | | | | | | | | | | | |
| 48278 | MAP2K3 | MKK3 | mitogen-activated | 3.2 | 2.8 | 1.1 | 1.0 | 1.0 | 1.0 | 1.1 | 1.0 | 1.0 | 1.0 | 4.0 |
| | | | protein kinase | | | | | | | | | | | |
| | | | kinase 3 | | | | | | | | | | | |
| 48740 | MAP2K6 | MKK6 | mitogen-activated | 0.6 | 1.4 | 1.5 | 1.5 | 0.9 | 1.1 | 1.3 | 1.2 | 1.0 | 1.0 | 4.7 |
| | | | protein kinase | | | | | | | | | | | |
| | | | kinase 6 | | | | | | | | | | | |
| 49284 | MAP2K6 | MKK6 | mitogen-activated | 0.6 | 1.2 | 1.5 | 1.4 | 0.9 | 1.1 | 1.3 | 1.0 | 1.0 | 1.0 | 4.5 |
| | | | protein kinase | | | | | | | | | | | |
| | | | kinase 6 | | | | | | | | | | | |
| 50045 | MAP3K5 | MKK5, ASK1 | mitogen-activated | 1.0 | 1.0 | 1.0 | 1.0 | 1.5 | 1.0 | [symbol] | 1.4 | 1.0 | 1.0 | 4.4 |
| | | | protein kinase | | | | | | | | | | | |
| | | | kinase kinase 5 | | | | | | | | | | | |
| 47969 | MAP3K8 | c-cot | mitogen-activated | 5.2 | 3.7 | 0.8 | 1.1 | 0.7 | 1.4 | 1.1 | 1.0 | 1.0 | 1.0 | 4.6 |
| | | | protein kinase | | | | | | | | | | | |
| | | | kinase kinase 8 | | | | | | | | | | | |
| 48424 | MAP4K4 | GCK-like, | mitogen-activated | 2.3 | 2.1 | 1.2 | 1.2 | 1.1 | 1.0 | 1.3 | 1.2 | 1.1 | 1.0 | 4.4 |
| | | HPK, HGK, | protein kinase | | | | | | | | | | | |
| | | NIK | kinase kinase kin | | | | | | | | | | | |
| 46998 | MAPK6 | MAPK6 | mitogen-activated | 2.0 | 1.7 | 1.0 | 1.0 | 0.9 | 1.0 | 0.9 | 1.0 | 0.9 | 1.1 | 4.1 |
| | | | protein kinase 6 | | | | | | | | | | | |
| 46965 | MAPK6 | MAPK6 | mitogen-activated | 2.0 | 1.7 | 1.0 | 1.0 | 0.9 | 1.0 | 0.9 | 1.0 | 0.9 | 1.0 | 4.1 |
| | | | protein kinase 6 | | | | | | | | | | | |
| 48377 | MAPK6 | MAPK6 | mitogen-activated | 2.1 | 1.9 | 1.1 | 1.0 | 0.9 | 1.0 | 1.0 | 1.0 | 0.9 | 1.0 | 4.0 |
| | | | protein kinase 6 | | | | | | | | | | | |
| 48410 | MAPK6 | MAPK6 | mitogen-activated | 2.1 | 2.0 | 1.1 | 1.0 | 1.0 | 1.0 | 1.0 | 1.0 | 0.9 | 1.0 | 4.0 |
| | | | protein kinase 6 | | | | | | | | | | | |
| 48992 | MAPKAPK2 | MAPKAPK2 | mitogen-activated | 1.0 | 1.0 | 1.4 | 1.3 | 1.1 | 1.0 | 1.2 | 1.1 | 1.0 | 1.0 | 4.4 |
| | | | protein kinase- | | | | | | | | | | | |
| | | | activated protein | | | | | | | | | | | |
| 50118 | MAPKAPK3 | MAPKAPK3 | mitogen-activated | 0.8 | 1.1 | 1.2 | 1.0 | 1.1 | 1.1 | [symbol] | 1.4 | 1.3 | 1.0 | 4.5 |
| | | | protein kinase- | | | | | | | | | | | |
| | | | activated protein | | | | | | | | | | | |
| 49511 | MATN2 | | | 0.6 | 1.3 | 1.5 | 1.4 | 0.9 | 1.0 | 1.5 | 1.2 | 0.9 | 1.0 | 4.7 |
| 48470 | [symbol] | | | 0.8 | 1.2 | 0.6 | 1.7 | 1.0 | 1.0 | [symbol] | 1.6 | 1.0 | 1.0 | 5.2 |
| 50742 | MGEA5 | MGEA5 | meningioma | 0.9 | 1.0 | 1.2 | 1.1 | 1.0 | 1.0 | [symbol] | 1.4 | 0.9 | 1.0 | 4.5 |
| | | | expressed antigen | | | | | | | | | | | |
| | | | 5 (hyaluronidase) | | | | | | | | | | | |
| 48933 | MMP2 | MMP2, | matrix | 1.0 | 1.0 | 1.3 | 1.0 | 1.2 | 1.0 | [symbol] | 1.3 | 1.3 | 1.2 | 4.5 |
| | | gelatinase A | metalloproteinase | | | | | | | | | | | |
| | | | 2 (gelatinase A, | | | | | | | | | | | |
| | | | 72 kD gel | | | | | | | | | | | |
| 48949 | MMP2 | MMP2, | matrix | 0.8 | 1.0 | 1.9 | 1.6 | 1.4 | 1.0 | 1.0 | 1.0 | 1.0 | 1.0 | 4.6 |
| | | gelatinase A | metalloproteinase | | | | | | | | | | | |
| | | | 2 (gelatinase A, | | | | | | | | | | | |
| | | | 72 kD gel | | | | | | | | | | | |
| 48334 | [symbol] 2 | MMP7 | matrix | 4.3 | 3.9 | 0.4 | 2.3 | 0.9 | 1.1 | [symbol] | 1.6 | 0.9 | 1.0 | 6.0 |
| | | | metalloproteinase | | | | | | | | | | | |
| | | | 7 (matrilysin, | | | | | | | | | | | |
| | | | uterine) | | | | | | | | | | | |
| 49124 | MMP7 | MMP7 | matrix | 3.2 | 3.1 | 0.6 | 1.6 | 0.9 | 1.0 | 0.7 | 1.0 | 1.0 | 1.0 | 4.6 |
| | | | metalloproteinase | | | | | | | | | | | |
| | | | 7 (matrilysin, | | | | | | | | | | | |
| | | | uterine) | | | | | | | | | | | |
| 46716 | MPHOSPH9 | | | 0.9 | 1.0 | 0.5 | 1.6 | 1.0 | 1.0 | 0.7 | 1.2 | 0.9 | 1.1 | 4.8 |
| 49860 | MSX1 | | | 1.2 | 1.1 | 1.2 | 1.1 | 1.0 | 1.0 | [symbol] | 1.6 | 1.1 | 1.1 | 4.8 |
| 47106 | MT1L | | | 2.0 | 1.7 | 1.5 | 1.0 | 0.8 | 1.0 | [symbol] 4 | 1.3 | 0.8 | 1.0 | 4.3 |
| 46766 | MT3 | | | 2.5 | 2.4 | 1.1 | 1.0 | 0.8 | 1.1 | 1.1 | 1.0 | 0.9 | 1.0 | 4.1 |
| 47772 | [symbol] 5 | | | 1.4 | 1.4 | 0.7 | 1.4 | 0.9 | 1.0 | [symbol] | 1.7 | 0.8 | 1.0 | 5.1 |
| 48399 | MUC1 | mucin1 | mucin 1, | 1.6 | 1.6 | 1.0 | 1.0 | 0.9 | 1.0 | 0.9 | 1.1 | 0.8 | 1.1 | 4.2 |
| | | | transmem-brane | | | | | | | | | | | |
| 50460 | MUC1 | mucin1 | mucin 1, | 2.0 | 1.8 | 1.0 | 1.0 | 0.8 | 1.1 | 0.9 | 1.0 | 0.8 | 1.1 | 4.2 |
| | | | transmem-brane | | | | | | | | | | | |
| 48332 | MUC4 | mucin4 | mucin 4, | 1.9 | 1.5 | 1.0 | 1.0 | 1.0 | 1.0 | 1.0 | 1.0 | 1.0 | 1.0 | 4.0 |
| | | | tracheobronchial | | | | | | | | | | | |
| 50406 | MX1 | | | 1.7 | 1.0 | 1.0 | 1.0 | 1.0 | 1.0 | 1.0 | 1.0 | [symbol] | 1.3 | 4.3 |
| 47754 | NBS1 | | | 2.4 | 1.7 | 0.9 | 1.0 | 1.1 | 1.0 | 0.9 | 1.0 | 1.1 | 1.1 | 4.1 |
| 47875 | NCK1 | Nck | NCK adaptor | 2.5 | 2.3 | 1.0 | 1.0 | 0.9 | 1.0 | 0.9 | 1.0 | 0.9 | 1.0 | 4.0 |
| | | | protein 1 | | | | | | | | | | | |
| 48719 | NCK1 | Nck | NCK adaptor | 2.1 | 1.9 | 1.0 | 1.0 | 1.0 | 1.0 | 0.9 | 1.0 | 1.0 | 1.0 | 4.0 |
| | | | protein 1 | | | | | | | | | | | |
| 48290 | NEDD4L | | | 1.8 | 1.6 | 1.1 | 1.0 | 1.1 | 1.0 | 1.1 | 1.0 | 1.0 | 1.0 | 4.0 |
| 46799 | NFE2L1 | NFE2L1 | nuclear factor | 2.1 | 1.5 | 1.0 | 1.0 | 1.0 | 1.0 | 1.5 | 1.2 | 1.0 | 1.0 | 4.2 |
| | | | (erythroid-derived | | | | | | | | | | | |
| | | | 2)-like 1 | | | | | | | | | | | |
| 47201 | NFIA | NFAT 3 | nuclear factor I/A | 0.6 | 1.5 | 1.3 | 1.2 | 1.0 | 1.0 | [symbol] | 1.4 | 1.0 | 1.0 | 4.6 |
| 47173 | NFKB1 | NFkappaB, | nuclear factor of | 6.3 | 6.1 | 1.2 | 1.0 | 1.0 | 1.0 | 1.0 | 1.0 | 0.9 | 1.0 | 4.0 |
| | | p105 | kappa light | | | | | | | | | | | |
| | | | polypeptide gene | | | | | | | | | | | |
| | | | enh | | | | | | | | | | | |
| 47877 | NFKB1 | NFkappaB, | nuclear factor of | 6.3 | 6.2 | 1.1 | 1.0 | 0.9 | 1.0 | 1.0 | 1.0 | 0.9 | 1.0 | 4.0 |
| | | p105 | kappa light | | | | | | | | | | | |
| | | | polypeptide gene | | | | | | | | | | | |
| | | | enh | | | | | | | | | | | |
| 50547 | NFKB2 | NFkappaB, | nuclear factor of | 5.3 | 3.1 | 1.1 | 1.1 | 1.0 | 1.0 | 1.1 | 1.0 | 0.9 | 1.0 | 4.1 |
| | | p49/p100 | kappa light | | | | | | | | | | | |
| | | | polypeptide gene | | | | | | | | | | | |
| | | | enh | | | | | | | | | | | |
| 50676 | NFKB2 | NFkappaB, | nuclear factor of | 4.6 | 2.4 | 1.1 | 1.0 | 1.0 | 1.0 | 1.1 | 1.0 | 0.9 | 1.0 | 4.0 |
| | | p49/p100 | kappa light | | | | | | | | | | | |
| | | | polypeptide gene | | | | | | | | | | | |
| | | | enh | | | | | | | | | | | |
| 49046 | NFKBIA | IkB, alpha | nuclear factor of | 9.4 | 6.3 | 1.6 | 1.4 | 0.9 | 1.1 | 1.1 | 1.1 | 1.0 | 1.0 | 4.5 |
| | | | kappa light | | | | | | | | | | | |
| | | | polypeptide gene | | | | | | | | | | | |
| | | | enh | | | | | | | | | | | |
| 47147 | NFKBIB | IkB, beta | nuclear factor of | 1.8 | 1.7 | 0.8 | 1.1 | 0.9 | 1.0 | 0.9 | 1.0 | 0.9 | 1.0 | 4.1 |
| | | | kappa light | | | | | | | | | | | |
| | | | polypeptide gene | | | | | | | | | | | |
| | | | enh | | | | | | | | | | | |
| 48923 | NFKBIB | IkB, beta | nuclear factor of | 1.7 | 1.7 | 0.9 | 1.0 | 1.0 | 1.0 | 0.9 | 1.0 | 1.0 | 1.0 | 4.0 |
| | | | kappa light | | | | | | | | | | | |
| | | | polypeptide gene | | | | | | | | | | | |
| | | | enh | | | | | | | | | | | |
| 50542 | NFKBIE | IkB, epsilon | nuclear factor of | 2.6 | 2.0 | 1.3 | 1.3 | 1.0 | 1.0 | 1.2 | 1.2 | 1.0 | 1.0 | 4.4 |
| | | | kappa light | | | | | | | | | | | |
| | | | polypeptide gene | | | | | | | | | | | |
| | | | enh | | | | | | | | | | | |
| 48405 | NFKBIE | IkB, epsilon | nuclear factor of | 2.8 | 2.7 | 1.3 | 1.3 | 1.1 | 1.0 | 1.3 | 1.1 | 1.1 | 1.0 | 4.4 |
| | | | kappa light | | | | | | | | | | | |
| | | | polypeptide gene | | | | | | | | | | | |
| | | | enh | | | | | | | | | | | |
| 49783 | [symbol] 6 | NFYB | nuclear | 0.9 | 1.0 | 0.6 | 1.5 | 0.8 | 1.1 | 0.7 | 1.1 | [symbol] | 1.4 | 5.0 |
| | | | transcription factor | | | | | | | | | | | |
| | | | Y, beta | | | | | | | | | | | |
| 49816 | [symbol] 6 | NFYB | nuclear | 0.9 | 1.0 | 0.6 | 1.4 | 0.7 | 1.2 | 0.8 | 1.1 | [symbol] | 1.4 | 5.2 |
| | | | transcription factor | | | | | | | | | | | |
| | | | Y, beta | | | | | | | | | | | |
| 48712 | NFYB | NFYB | nuclear | 0.8 | 1.1 | 0.7 | 1.0 | 0.8 | 1.1 | 0.7 | 1.1 | [symbol] | 1.3 | 4.6 |
| | | | transcription factor | | | | | | | | | | | |
| | | | Y, beta | | | | | | | | | | | |
| 49690 | NID | NID | nidogen (enactin) | 0.9 | 1.0 | 1.8 | 1.5 | 1.3 | 1.0 | [symbol] 0 | 1.4 | 1.0 | 1.0 | 4.9 |
| 49014 | NINJ1 | | | 3.5 | 2.9 | 1.1 | 1.0 | 1.1 | 1.1 | 1.2 | 1.0 | 1.2 | 1.0 | 4.2 |
| 48029 | NOL3 | NOL3 | nucleolar protein 3 | 0.5 | 2.0 | 0.8 | 1.1 | 0.9 | 1.0 | 1.0 | 1.0 | 1.1 | 1.0 | 4.2 |
| | | | (apoptosis | | | | | | | | | | | |
| | | | repressor with | | | | | | | | | | | |
| | | | CARD | | | | | | | | | | | |
| 47679 | NR4A2 | TINUR | nuclear receptor | 1.3 | 1.0 | 1.7 | 1.6 | 0.8 | 1.0 | 1.3 | 1.0 | 0.8 | 1.1 | 4.7 |
| | | | subfamily 4, group | | | | | | | | | | | |
| | | | A, member 2 | | | | | | | | | | | |
| 47897 | NR4A2 | TINUR | nuclear receptor | 1.6 | 1.1 | 1.9 | 1.4 | 0.9 | 1.0 | 1.5 | 1.1 | 0.9 | 1.0 | 4.5 |
| | | | subfamily 4, group | | | | | | | | | | | |
| | | | A, member 2 | | | | | | | | | | | |
| 47712 | NR4A2 | TINUR | nuclear receptor | 1.4 | 1.0 | 1.7 | 1.5 | 0.7 | 1.1 | 1.5 | 1.1 | 1.0 | 1.0 | 4.7 |
| | | | subfamily 4, group | | | | | | | | | | | |
| | | | A, member 2 | | | | | | | | | | | |
| 48227 | [symbol] 7 | neuregulin1, | neuregulin 1 | 1.3 | 1.1 | 0.6 | 1.5 | 0.9 | 1.1 | [symbol] | 1.4 | 0.9 | 1.1 | 5.0 |
| | | neu diff. | | | | | | | | | | | | |
| | | Factor, | | | | | | | | | | | | |
| | | heregulin | | | | | | | | | | | | |
| 48454 | NRP1 | | | 0.5 | 1.6 | 1.1 | 1.0 | 1.1 | 1.0 | 1.1 | 1.0 | 1.1 | 1.0 | 4.0 |
| 47416 | NTS | | | 0.7 | 1.2 | 0.6 | 1.4 | 0.9 | 1.0 | 1.0 | 1.0 | 0.9 | 1.0 | 4.4 |
| 49778 | OSMR | oncostatin MR | oncostatin M | 2.2 | 1.8 | 1.4 | 1.2 | 1.1 | 1.0 | [symbol] | 1.3 | 1.0 | 1.0 | 4.5 |
| | | | receptor | | | | | | | | | | | |
| 47183 | OSMR | oncostatin MR | oncostatin M | 2.6 | 2.5 | 1.5 | 1.5 | 1.0 | 1.0 | 1.3 | 1.1 | 1.1 | 1.0 | 4.6 |
| | | | receptor | | | | | | | | | | | |
| 48545 | P2RY1 | P2RY1 | purinergic | 0.8 | 1.1 | 1.0 | 1.0 | 1.3 | 1.2 | 0.9 | 1.0 | [symbol] | 1.4 | 4.6 |
| | | | receptor P2Y, G- | | | | | | | | | | | |
| | | | protein coupled, 1 | | | | | | | | | | | |
| 48193 | P2RY1 | P2RY1 | purinergic | 1.0 | 1.0 | 0.8 | 1.0 | 1.1 | 1.0 | 1.2 | 1.0 | [symbol] | 1.4 | 4.4 |
| | | | receptor P2Y, G- | | | | | | | | | | | |
| | | | protein coupled, 1 | | | | | | | | | | | |
| 46650 | P2RY6 | P2RY6 | pyrimidinergic | 0.6 | 1.7 | 1.1 | 1.1 | 0.9 | 1.0 | 1.3 | 1.2 | 0.8 | 1.0 | 4.3 |
| | | | receptor P2Y, G- | | | | | | | | | | | |
| | | | protein coupled, 6 | | | | | | | | | | | |
| 47194 | P2RY6 | P2RY6 | pyrimidinergic | 0.5 | 1.7 | 1.2 | 1.1 | 0.9 | 1.0 | 1.5 | 1.2 | 1.0 | 1.0 | 4.4 |
| | | | receptor P2Y, G- | | | | | | | | | | | |
| | | | protein coupled, 6 | | | | | | | | | | | |
| 48707 | P4HA1 | P4HA1, prolyl- | procollagen- | 1.0 | 1.0 | 1.6 | 1.1 | 0.9 | 1.0 | [symbol] | 1.5 | 0.9 | 1.0 | 4.7 |
| | | 4-hydroxylase | proline, 2- | | | | | | | | | | | |
| | | alpha1 | oxoglutarate 4- | | | | | | | | | | | |
| | | | dioxygenase | | | | | | | | | | | |
| 47935 | P4HA1 | P4HA1, prolyl- | procollagen- | 0.9 | 1.0 | 1.5 | 1.2 | 0.8 | 1.1 | [symbol] | 1.4 | 1.0 | 1.0 | 4.7 |
| | | 4-hydroxylase | proline, 2- | | | | | | | | | | | |
| | | alpha1 | oxoglutarate 4- | | | | | | | | | | | |
| | | | dioxygenase | | | | | | | | | | | |
| 48971 | [symbol] 8 | P4HA2 | procollagen- | 1.1 | 1.0 | 2.1 | 1.4 | 1.0 | 1.0 | [symbol] | 1.6 | 1.2 | 1.1 | 5.2 |
| | | | proline, 2- | | | | | | | | | | | |
| | | | oxoglutarate 4- | | | | | | | | | | | |
| | | | dioxygenase | | | | | | | | | | | |
| 49905 | PAX3 | | | 1.9 | 1.6 | 1.1 | 1.0 | 0.9 | 1.0 | 1.1 | 1.0 | 1.0 | 1.0 | 4.0 |
| 49705 | PCYT2 | | | 0.5 | 1.7 | 0.9 | 1.0 | 1.0 | 1.0 | 1.2 | 1.0 | 0.9 | 1.0 | 4.0 |
| 48556 | PDE4B | phosphodiesterase | phosphodiesterase | 2.6 | 2.5 | 1.2 | 1.1 | 1.0 | 1.0 | 1.0 | 1.0 | 1.1 | 1.0 | 4.2 |
| | | 4B | 4B, cAMP- | | | | | | | | | | | |
| | | | specific (dunce | | | | | | | | | | | |
| | | | (Drosop | | | | | | | | | | | |
| 48427 | PDE4B | phosphodiesterase | phosphodiesterase | 2.7 | 2.3 | 1.2 | 1.2 | 1.0 | 1.0 | 1.1 | 1.0 | 1.2 | 1.0 | 4.2 |
| | | 4B | 4B, cAMP- | | | | | | | | | | | |
| | | | specific (dunce | | | | | | | | | | | |
| | | | (Drosop | | | | | | | | | | | |
| 49671 | [symbol] 9 | PDEF | prostate | 1.0 | 1.0 | 1.5 | 1.2 | 1.0 | 1.0 | [symbol] | 1.9 | 1.0 | 1.0 | 5.1 |
| | | | epithelium-specific | | | | | | | | | | | |
| | | | Ets transcription | | | | | | | | | | | |
| | | | fac | | | | | | | | | | | |
| 48370 | PDGFA | PDGF, alpha | platelet-derived | 0.9 | 1.0 | 1.1 | 1.0 | 1.2 | 1.0 | [symbol] | 1.4 | 1.3 | 1.1 | 4.5 |
| | | | growth factor | | | | | | | | | | | |
| | | | alpha polypeptide | | | | | | | | | | | |
| 48824 | PDK2 | | | 0.5 | 1.6 | 0.8 | 1.2 | 1.0 | 1.0 | 0.8 | 1.0 | 1.1 | 1.0 | 4.2 |
| 49965 | PF4 | platelet factor 4 | platelet factor 4 | 2.3 | 1.5 | 1.0 | 1.0 | 1.2 | 1.0 | 1.1 | 1.0 | 1.0 | 1.0 | 4.0 |
| 47320 | PI3 | PI3 | protease inhibitor | 18.5 | 17.4 | 1.0 | 1.0 | 1.0 | 1.0 | 0.9 | 1.0 | 1.2 | 1.0 | 4.0 |
| | | | 3, skin-derived | | | | | | | | | | | |
| | | | (SKALP) | | | | | | | | | | | |
| 49975 | PIM1 | pim-1 | pim-1 oncogene | 1.5 | 1.4 | 0.8 | 1.2 | 1.0 | 1.0 | [symbol] | 1.3 | 0.9 | 1.0 | 4.5 |
| 46818 | PIM2 | Pim-2 | pim-2 oncogene | 3.7 | 1.7 | 1.1 | 1.1 | 0.9 | 1.0 | [symbol] | 1.5 | 1.2 | 1.1 | 4.6 |
| 50338 | PIM2 | Pim-2 | pim-2 oncogene | 2.4 | 1.8 | 1.2 | 1.1 | 0.9 | 1.0 | [symbol] | 1.4 | 1.0 | 1.0 | 4.5 |
| 48547 | PLA2G1B | phospholipase | phospholipase A2, | 1.0 | 1.0 | 1.0 | 1.0 | 0.6 | 1.7 | 0.8 | 1.0 | 0.8 | 1.0 | 4.7 |
| | | A2 | group IB | | | | | | | | | | | |
| | | | (pancreas) | | | | | | | | | | | |
| 46577 | PLA2G4B | phospholipase | phospholipase A2, | 1.2 | 1.1 | 0.6 | 1.5 | 0.9 | 1.0 | 0.7 | 1.2 | 0.9 | 1.0 | 4.7 |
| | | A2 | group IVB | | | | | | | | | | | |
| | | | (cytosolic) | | | | | | | | | | | |
| 48624 | PLA2R1 | phospholipase | phospholipase A2 | 1.0 | 1.0 | 1.4 | 1.0 | 0.9 | 1.0 | [symbol] 0 | 1.5 | 1.0 | 1.0 | 4.5 |
| | | A2 | receptor 1, 180 kD | | | | | | | | | | | |
| 48023 | PLAB | PLAB | prostate | 2.4 | 2.1 | 1.2 | 1.1 | 1.2 | 1.1 | 1.2 | 1.1 | 1.2 | 1.0 | 4.2 |
| | | | differentiation | | | | | | | | | | | |
| | | | factor | | | | | | | | | | | |
| 50434 | PLAB | PLAB | prostate | 2.0 | 1.7 | 1.2 | 1.1 | 1.1 | 1.1 | 1.1 | 1.0 | 1.0 | 1.0 | 4.2 |
| | | | differentiation | | | | | | | | | | | |
| | | | factor | | | | | | | | | | | |
| 49424 | PLAU | urokinase-type | plasminogen | 7.9 | 6.6 | 1.5 | 1.3 | 1.4 | 1.3 | 1.1 | 1.0 | 1.3 | 1.0 | 4.6 |
| | | plasminogen | activator, | | | | | | | | | | | |
| | | activator | urokinase | | | | | | | | | | | |
| 47537 | PLAU | urokinase-type | plasminogen | 9.1 | 7.2 | 1.4 | 1.2 | 1.3 | 1.3 | 1.0 | 1.0 | 1.2 | 1.0 | 4.5 |
| | | plasminogen | activator, | | | | | | | | | | | |
| | | activator | urokinase | | | | | | | | | | | |
| 48138 | [symbol] | | | 2.2 | 2.0 | 1.4 | 1.2 | 1.0 | 1.0 | [symbol] | 1.7 | 1.2 | 1.2 | 5.1 |
| 49422 | PLCG1 | PLC gamma | phospholipase C, | 0.6 | 1.5 | 0.9 | 1.1 | 0.9 | 1.0 | 0.9 | 1.0 | 1.0 | 1.0 | 4.2 |
| | | 1, | gamma 1 | | | | | | | | | | | |
| | | phopholipase | (formerly subtype | | | | | | | | | | | |
| | | C, gamma 1 | 148) | | | | | | | | | | | |
| 49073 | [symbol] | PLOD | procollagen- | 0.8 | 1.2 | 1.7 | 1.6 | 1.0 | 1.0 | [symbol] | 1.5 | 1.0 | 1.0 | 5.1 |
| | | | lysine, 2- | | | | | | | | | | | |
| | | | oxoglutarate 5- | | | | | | | | | | | |
| | | | dioxygenase ( | | | | | | | | | | | |
| 47704 | [symbol] | PLOD | procollagen- | 0.8 | 1.2 | 1.6 | 1.5 | 1.1 | 1.0 | [symbol] | 1.5 | 1.1 | 1.0 | 5.0 |
| | | | lysine, 2- | | | | | | | | | | | |
| | | | oxoglutarate 5- | | | | | | | | | | | |
| | | | dioxygenase ( | | | | | | | | | | | |
| 49106 | PLOD | PLOD | procollagen- | 0.8 | 1.1 | 1.7 | 1.5 | 1.0 | 1.0 | [symbol] | 1.4 | 1.0 | 1.0 | 4.9 |
| | | | lysine, 2- | | | | | | | | | | | |
| | | | oxoglutarate 5- | | | | | | | | | | | |
| | | | dioxygenase ( | | | | | | | | | | | |
| 47671 | PLOD | PLOD | procollagen- | 0.8 | 1.2 | 1.6 | 1.4 | 1.0 | 1.0 | [symbol] | 1.3 | 1.0 | 1.0 | 4.7 |
| | | | lysine, 2- | | | | | | | | | | | |
| | | | oxoglutarate 5- | | | | | | | | | | | |
| | | | dioxygenase ( | | | | | | | | | | | |
| 50313 | PLOD2 | PLOD2 | procollagen- | 1.0 | 1.0 | 1.5 | 1.1 | 0.8 | 1.0 | [symbol] 4 | 1.3 | 1.0 | 1.0 | 4.5 |
| | | | lysine, 2- | | | | | | | | | | | |
| | | | oxoglutarate 5- | | | | | | | | | | | |
| | | | dioxygenase ( | | | | | | | | | | | |
| 49325 | PLOD2 | PLOD2 | procollagen- | 1.1 | 1.0 | 1.5 | 1.0 | 0.8 | 1.0 | [symbol] | 1.3 | 1.0 | 1.0 | 4.4 |
| | | | lysine, 2- | | | | | | | | | | | |
| | | | oxoglutarate 5- | | | | | | | | | | | |
| | | | dioxygenase ( | | | | | | | | | | | |
| 49809 | PLXNB1 | PLXNB1 | plexin B1 | 0.6 | 1.6 | 0.8 | 1.0 | 0.9 | 1.1 | 0.8 | 1.0 | [symbol] | 1.3 | 4.3 |
| 49950 | PPAP2B | | | 3.0 | 2.3 | 1.0 | 1.0 | 1.1 | 1.0 | 1.3 | 1.0 | 1.0 | 1.0 | 4.0 |
| 46862 | PPIF | cyclophilin F | peptidylprolyl | 1.9 | 1.7 | 1.1 | 1.0 | 1.1 | 1.1 | 1.1 | 1.0 | 1.0 | 1.0 | 4.2 |
| | | | isomerase F | | | | | | | | | | | |
| | | | (cyclophilin F) | | | | | | | | | | | |
| 48616 | PPIH | cyclophilin H | peptidyl prolyl | 0.8 | 1.1 | 0.6 | 1.4 | 0.9 | 1.0 | 0.8 | 1.1 | 1.0 | 1.0 | 4.5 |
| | | | isomerase H | | | | | | | | | | | |
| | | | (cyclophilin H) | | | | | | | | | | | |
| 49738 | PRKCA | PKC alpha | protein kinase C, | 0.9 | 1.0 | 1.5 | 1.4 | 1.0 | 1.0 | [symbol] | 1.3 | 1.1 | 1.0 | 4.8 |
| | | | alpha | | | | | | | | | | | |
| 48579 | PRKCBP1 | PRKCBP1 | protein kinase C | 1.3 | 1.3 | 0.7 | 1.2 | 0.8 | 1.1 | [symbol] | 1.5 | 0.8 | 1.1 | 4.9 |
| | | | binding protein 1 | | | | | | | | | | | |
| 47190 | PRKCE | PKC epsilon | protein kinase C, | 1.0 | 1.0 | 1.5 | 1.3 | 1.0 | 1.0 | 1.0 | 1.0 | 1.0 | 1.0 | 4.3 |
| | | | epsilon | | | | | | | | | | | |
| 49921 | PRKR | | | 1.8 | 1.6 | 1.3 | 1.1 | 1.0 | 1.0 | [symbol] | 1.3 | 1.4 | 1.0 | 4.4 |
| 50401 | PROL3 | | | 0.9 | 1.0 | 1.0 | 1.0 | 1.0 | 1.0 | 0.9 | 1.0 | [symbol] | 1.3 | 4.3 |
| 50096 | PRSS11 | | | 0.7 | 1.5 | 1.6 | 1.3 | 1.1 | 1.1 | [symbol] | 1.4 | 1.2 | 1.1 | 4.9 |
| 49475 | PSA | | | 0.8 | 1.1 | 0.9 | 1.0 | 1.0 | 1.0 | [symbol] | 1.3 | 0.8 | 1.2 | 4.5 |
| 49219 | PSMB10 | | | 2.8 | 2.2 | 0.9 | 1.0 | 1.1 | 1.0 | 1.0 | 1.0 | 1.2 | 1.0 | 4.0 |
| 47998 | PSMB9 | | | 2.6 | 1.7 | 1.1 | 1.0 | 1.2 | 1.0 | 1.3 | 1.0 | 1.2 | 1.0 | 4.0 |
| 50625 | PSMC6 | | | 1.3 | 1.1 | 0.6 | 1.4 | 0.9 | 1.0 | 0.8 | 1.0 | 0.8 | 1.1 | 4.6 |
| 47830 | PSME1 | | | 1.6 | 1.6 | 1.1 | 1.0 | 1.1 | 1.0 | 0.9 | 1.1 | 1.0 | 1.0 | 4.1 |
| 49846 | PTEN | PTEN | phosphatase and | 0.7 | 1.3 | 1.4 | 1.2 | 1.0 | 1.0 | [symbol] 4 | 1.3 | 0.9 | 1.0 | 4.6 |
| | | | tensin homolog | | | | | | | | | | | |
| | | | (mutated in multipl | | | | | | | | | | | |
| 47995 | [symbol] | PTGER1 | prostaglandin E | 1.2 | 1.0 | 0.7 | 1.4 | 0.9 | 1.1 | [symbol] | 1.4 | 0.8 | 1.1 | 5.0 |
| | | | receptor 1 | | | | | | | | | | | |
| | | | (subtype EP1), | | | | | | | | | | | |
| | | | 42 kD | | | | | | | | | | | |
| 50200 | PTGER4 | EP2 | prostaglandin E | 1.1 | 1.0 | 1.0 | 1.0 | 1.2 | 1.0 | [symbol] 4 | 1.3 | 1.1 | 1.0 | 4.3 |
| | | | receptor 4 | | | | | | | | | | | |
| | | | (subtype EP4) | | | | | | | | | | | |
| 46632 | [symbol] | PTGES | prostaglandin E | 3.2 | 3.1 | 2.4 | 1.9 | 1.2 | 1.0 | [symbol] | 1.6 | 1.2 | 1.0 | 5.5 |
| | | | synthase | | | | | | | | | | | |
| 47176 | [symbol] | PTGES | prostaglandin E | 3.5 | 3.4 | 2.6 | 2.0 | 1.2 | 1.1 | [symbol] 0 | 1.3 | 1.1 | 1.0 | 5.4 |
| | | | synthase | | | | | | | | | | | |
| 50010 | PTGIR | PTGIR | prostaglandin I2 | 0.7 | 1.0 | 0.7 | 1.1 | 1.0 | 1.0 | [symbol] | 1.3 | 0.8 | 1.0 | 4.5 |
| | | | (prostacyclin) | | | | | | | | | | | |
| | | | receptor (IP) | | | | | | | | | | | |
| 47499 | PTGS2 | PTGS2, COX2 | prostaglandin- | 14.9 | 7.0 | 1.2 | 1.1 | 0.9 | 1.0 | 0.9 | 1.0 | 0.9 | 1.0 | 4.1 |
| | | | endoperoxide | | | | | | | | | | | |
| | | | synthase 2 | | | | | | | | | | | |
| | | | (prostagland | | | | | | | | | | | |
| 50665 | PTGS2 | PTGS2, COX2 | prostaglandin- | 20.4 | 10.9 | 1.1 | 1.0 | 0.9 | 1.0 | 0.9 | 1.0 | 0.9 | 1.0 | 4.0 |
| | | | endoperoxide | | | | | | | | | | | |
| | | | synthase 2 | | | | | | | | | | | |
| | | | (prostagland | | | | | | | | | | | |
| 50376 | PTPN1 | PTP1B | protein tyrosine | 2.0 | 1.8 | 1.1 | 1.0 | 1.1 | 1.0 | 1.2 | 1.1 | 1.1 | 1.0 | 4.2 |
| | | | phosphatase, non- | | | | | | | | | | | |
| | | | receptor type 1 | | | | | | | | | | | |
| 48259 | PTPN1 | PTP1B | protein tyrosine | 2.1 | 2.1 | 1.1 | 1.0 | 1.0 | 1.0 | 1.3 | 1.2 | 0.9 | 1.0 | 4.2 |
| | | | phosphatase, non- | | | | | | | | | | | |
| | | | receptor type 1 | | | | | | | | | | | |
| 47208 | PTPN3 | PTPN3 | protein tyrosine | 0.9 | 1.0 | 1.4 | 1.3 | 1.0 | 1.0 | 1.4 | 1.2 | 1.0 | 1.0 | 4.6 |
| | | | phosphatase, non- | | | | | | | | | | | |
| | | | receptor type 3 | | | | | | | | | | | |
| 50395 | PTPRF | PTPRF | protein tyrosine | 0.9 | 1.1 | 1.4 | 1.3 | 1.4 | 1.2 | 1.4 | 1.1 | 1.3 | 1.1 | 4.7 |
| | | | phosphatase, | | | | | | | | | | | |
| | | | receptor type, F | | | | | | | | | | | |
| 49533 | PTPRG | PTPRG | protein tyrosine | 0.9 | 1.0 | 1.5 | 1.3 | 1.1 | 1.0 | [symbol] | 1.5 | 1.0 | 1.0 | 4.8 |
| | | | phosphatase, | | | | | | | | | | | |
| | | | receptor type, G | | | | | | | | | | | |
| 50348 | PTPRG | PTPRG | protein tyrosine | 0.9 | 1.0 | 1.7 | 1.6 | 1.3 | 1.1 | 1.0 | 1.0 | 1.0 | 1.0 | 4.7 |
| | | | phosphatase, | | | | | | | | | | | |
| | | | receptor type, G | | | | | | | | | | | |
| 47698 | PTPRJ | PTPRJ | protein tyrosine | 1.7 | 1.6 | 1.6 | 1.5 | 1.0 | 1.0 | 1.3 | 1.2 | 1.0 | 1.0 | 4.7 |
| | | | phosphatase, | | | | | | | | | | | |
| | | | receptor type, J | | | | | | | | | | | |
| 47308 | PTPRJ | PTPRJ | protein tyrosine | 1.5 | 1.4 | 1.6 | 1.4 | 1.0 | 1.0 | 1.3 | 1.2 | 1.1 | 1.0 | 4.6 |
| | | | phosphatase, | | | | | | | | | | | |
| | | | receptor type, J | | | | | | | | | | | |
| 50632 | PTPRJ | PTPRJ | protein tyrosine | 1.6 | 1.4 | 1.5 | 1.4 | 1.0 | 1.0 | [symbol] | 1.3 | 1.0 | 1.0 | 4.7 |
| | | | phosphatase, | | | | | | | | | | | |
| | | | receptor type, J | | | | | | | | | | | |
| 47665 | PTPRJ | PTPRJ | protein tyrosine | 2.0 | 1.8 | 1.6 | 1.5 | 1.0 | 1.0 | 1.2 | 1.0 | 1.0 | 1.0 | 4.5 |
| | | | phosphatase, | | | | | | | | | | | |
| | | | receptor type, J | | | | | | | | | | | |
| 47983 | PTPRN2 | PTPRN2 | protein tyrosine | 0.9 | 1.0 | 0.5 | 1.4 | 1.0 | 1.0 | 0.6 | 1.0 | 1.0 | 1.0 | 4.4 |
| | | | phosphatase, | | | | | | | | | | | |
| | | | receptor type, N | | | | | | | | | | | |
| | | | pol | | | | | | | | | | | |
| 46924 | RAB9 | RAB9 | RAB9, member | 2.3 | 2.0 | 1.2 | 1.2 | 1.0 | 1.0 | 1.1 | 1.0 | 1.0 | 1.0 | 4.2 |
| | | | RAS oncogene | | | | | | | | | | | |
| | | | family | | | | | | | | | | | |
| 47002 | RAC1 | Rac-1 | ras-related C3 | 2.4 | 2.2 | 1.1 | 1.0 | 1.1 | 1.1 | 1.0 | 1.0 | 1.0 | 1.0 | 4.1 |
| | | | botulinum toxin | | | | | | | | | | | |
| | | | substrate 1 (rho fa | | | | | | | | | | | |
| 46969 | RAC1 | Rac-1 | ras-related C3 | 2.4 | 2.2 | 1.1 | 1.1 | 1.1 | 1.0 | 1.0 | 1.0 | 1.0 | 1.0 | 4.1 |
| | | | botulinum toxin | | | | | | | | | | | |
| | | | substrate 1 (rho fa | | | | | | | | | | | |
| 47319 | RAC1 | Rac-1 | ras-related C3 | 2.5 | 2.3 | 1.2 | 1.0 | 1.1 | 1.0 | 0.9 | 1.0 | 1.1 | 1.0 | 4.0 |
| | | | botulinum toxin | | | | | | | | | | | |
| | | | substrate 1 (rho fa | | | | | | | | | | | |
| 48542 | RAP1GA1 | | | 0.4 | 1.9 | 1.0 | 1.0 | 1.0 | 1.0 | 1.2 | 1.0 | 1.2 | 1.0 | 4.0 |
| 47707 | RASA2 | RasGAP | RAS p21 protein | 1.6 | 1.5 | 0.9 | 1.0 | 0.9 | 1.0 | 1.0 | 1.0 | 1.1 | 1.0 | 4.0 |
| | | | activator 2 | | | | | | | | | | | |
| 47652 | RELA | p65, NFKB3 | v-rel avian | 2.4 | 2.0 | 0.8 | 1.1 | 1.0 | 1.0 | 1.0 | 1.0 | 1.0 | 1.0 | 4.1 |
| | | | reticuloendotheliosis | | | | | | | | | | | |
| | | | viral oncogene h | | | | | | | | | | | |
| 47672 | RELA | p65, NFKB3 | v-rel avian | 2.5 | 2.0 | 0.9 | 1.0 | 1.1 | 1.0 | 0.9 | 1.0 | 1.1 | 1.0 | 4.0 |
| | | | reticuloendotheliosis | | | | | | | | | | | |
| | | | viral oncogene h | | | | | | | | | | | |
| 49087 | RELB | l-rel | v-rel avian | 7.1 | 6.4 | 1.3 | 1.0 | 1.1 | 1.0 | 0.9 | 1.0 | 1.0 | 1.0 | 4.0 |
| | | | reticuloendotheliosis | | | | | | | | | | | |
| | | | viral oncogene h | | | | | | | | | | | |
| 49120 | RELB | l-rel | v-rel avian | 7.1 | 6.2 | 1.3 | 1.0 | 1.1 | 1.0 | 1.0 | 1.0 | 1.1 | 1.0 | 4.0 |
| | | | reticuloendotheliosis | | | | | | | | | | | |
| | | | viral oncogene h | | | | | | | | | | | |
| 46658 | RELB | l-rel | v-rel avian | 2.7 | 2.3 | 1.0 | 1.0 | 1.0 | 1.0 | 1.0 | 1.0 | 1.0 | 1.0 | 4.0 |
| | | | reticuloendotheliosis | | | | | | | | | | | |
| | | | viral oncogene h | | | | | | | | | | | |
| 49813 | RGS14 | RGS14 | regulator of G- | 0.3 | 2.7 | 0.9 | 1.0 | 1.0 | 1.0 | 1.2 | 1.1 | 1.1 | 1.0 | 4.1 |
| | | | protein signalling | | | | | | | | | | | |
| | | | 14 | | | | | | | | | | | |
| 47933 | RIPK2 | RIP2, RICK | receptor- | 4.7 | 3.9 | 1.2 | 1.1 | 1.1 | 1.0 | 1.1 | 1.0 | 1.1 | 1.0 | 4.1 |
| | | | interacting serine- | | | | | | | | | | | |
| | | | threonine kinase 2 | | | | | | | | | | | |
| 48244 | RPS6KA5 | RPS6KA5 | ribosomal protein | 0.8 | 1.1 | 0.8 | 1.1 | 1.0 | 1.0 | [symbol] | 1.3 | 1.0 | 1.0 | 4.5 |
| | | | S6 kinase, 90 kD, | | | | | | | | | | | |
| | | | polypeptide 5 | | | | | | | | | | | |
| 50666 | S100A12 | S100A12 | S100 calcium- | 4.1 | 3.3 | 0.8 | 1.0 | 1.0 | 1.0 | 0.9 | 1.0 | 1.1 | 1.0 | 4.0 |
| | | | binding protein | | | | | | | | | | | |
| | | | A12 (calgranulin | | | | | | | | | | | |
| | | | C) | | | | | | | | | | | |
| 46891 | SAA2 | | | 1.0 | 1.0 | 1.6 | 1.4 | 1.0 | 1.0 | 1.4 | 1.0 | 0.9 | 1.0 | 4.4 |
| 49076 | [symbol] | KIAA0524 | KIAA0524 protein | 0.8 | 1.1 | 0.7 | 1.3 | 0.7 | 1.4 | 0.8 | 1.0 | [symbol] | 1.4 | 5.1 |
| 50441 | SAT | | | 3.3 | 3.2 | 0.7 | 1.4 | 0.7 | 1.2 | 0.8 | 1.0 | 0.8 | 1.0 | 4.6 |
| 49507 | SCNN1A | | | 0.6 | 1.6 | 0.8 | 1.0 | 0.9 | 1.0 | 1.0 | 1.0 | 0.9 | 1.0 | 4.0 |
| 50026 | SCYA2 | MCP1 | small inducible | 100.5 | 99.1 | 1.0 | 1.0 | 1.0 | 1.0 | [symbol] | 1.4 | 1.1 | 1.0 | 4.4 |
| | | | cytokine A2 | | | | | | | | | | | |
| | | | (monocyte | | | | | | | | | | | |
| | | | chemotactic | | | | | | | | | | | |
| 47916 | SCYA2 | MCP1 | small inducible | 1.0 | 1.0 | 1.0 | 1.0 | 1.0 | 1.0 | [symbol] | 1.4 | 1.0 | 1.0 | 4.4 |
| | | | cytokine A2 | | | | | | | | | | | |
| | | | (monocyte | | | | | | | | | | | |
| | | | chemotactic | | | | | | | | | | | |
| 48830 | SCYA2 | MCP1 | small inducible | 16.0 | 8.8 | 1.1 | 1.0 | 0.9 | 1.0 | [symbol] | 1.3 | 1.0 | 1.0 | 4.3 |
| | | | cytokine A2 | | | | | | | | | | | |
| | | | (monocyte | | | | | | | | | | | |
| | | | chemotactic | | | | | | | | | | | |
| 46596 | SCYA20 | MIP-3a | small inducible | 1.8 | 1.7 | 0.9 | 1.0 | 1.0 | 1.0 | 0.8 | 1.0 | 1.0 | 1.0 | 4.0 |
| | | | cytokine subfamily | | | | | | | | | | | |
| | | | A (Cys—Cys), me | | | | | | | | | | | |
| 48190 | SCYA20 | MIP-3a | small inducible | 328.2 | 325.5 | 1.6 | 1.5 | 1.0 | 1.0 | 0.9 | 1.0 | 0.8 | 1.0 | 4.5 |
| | | | cytokine subfamily | | | | | | | | | | | |
| | | | A (Cys—Cys), me | | | | | | | | | | | |
| 46598 | SCYA20 | MIP-3a | small inducible | 275.9 | 218.3 | 1.0 | 1.0 | 1.0 | 1.0 | 0.8 | 1.0 | 1.0 | 1.0 | 4.0 |
| | | | cytokine subfamily | | | | | | | | | | | |
| | | | A (Cys—Cys), me | | | | | | | | | | | |
| 47893 | SCYA4 | MIP-1beta | small inducible | 5.1 | 2.5 | 1.0 | 1.0 | 1.0 | 1.0 | 0.9 | 1.0 | 1.1 | 1.0 | 4.0 |
| | | | cytokine A4 | | | | | | | | | | | |
| | | | (homologous to | | | | | | | | | | | |
| | | | mouse M | | | | | | | | | | | |
| 48257 | SCYA4 | MIP-1beta | small inducible | 4.4 | 3.7 | 1.0 | 1.0 | 1.0 | 1.0 | 0.9 | 1.0 | 1.1 | 1.0 | 4.0 |
| | | | cytokine A4 | | | | | | | | | | | |
| | | | (homologous to | | | | | | | | | | | |
| | | | mouse M | | | | | | | | | | | |
| 49839 | SCYB5 | ENA-78 | small inducible | 27.5 | 18.0 | 0.9 | 1.0 | 1.0 | 1.0 | 1.1 | 1.0 | 0.9 | 1.0 | 4.0 |
| | | | cytokine subfamily | | | | | | | | | | | |
| | | | B (Cys-X-Cys), | | | | | | | | | | | |
| 49968 | SCYB5 | ENA-78 | small inducible | 26.3 | 16.6 | 1.1 | 1.0 | 1.0 | 1.0 | 1.1 | 1.0 | 0.9 | 1.0 | 4.0 |
| | | | cytokine subfamily | | | | | | | | | | | |
| | | | B (Cys-X-Cys), | | | | | | | | | | | |
| 49645 | SCYD1 | fractalkine | small inducible | 14.5 | 5.8 | 1.0 | 1.0 | 1.0 | 1.0 | [symbol] | 1.3 | 1.4 | 1.2 | 4.5 |
| | | | cytokine subfamily | | | | | | | | | | | |
| | | | D (Cys-X3-Cys), | | | | | | | | | | | |
| 49492 | SCYD1 | fractalkine | small inducible | 4.5 | 2.3 | 1.1 | 1.0 | 1.2 | 1.0 | 0.8 | 1.0 | 1.4 | 1.2 | 4.2 |
| | | | cytokine subfamily | | | | | | | | | | | |
| | | | D (Cys-X3-Cys), | | | | | | | | | | | |
| 48646 | SDCBP | | | 2.6 | 2.5 | 1.2 | 1.1 | 1.0 | 1.0 | 1.2 | 1.0 | 1.0 | 1.0 | 4.1 |
| 49498 | [symbol] | | serine (or | 11.3 | 9.5 | 0.7 | 1.1 | 0.9 | 1.0 | [symbol] | 2.2 | 0.9 | 1.0 | 5.3 |
| | | | cysteine) | | | | | | | | | | | |
| | | | proteinase | | | | | | | | | | | |
| | | | inhibitor, clade A | | | | | | | | | | | |
| 46950 | [symbol] | | serine (or | 20.4 | 14.0 | 0.6 | 1.6 | 0.9 | 1.1 | [symbol] | 2.0 | 0.9 | 1.0 | 5.7 |
| | | | cysteine) | | | | | | | | | | | |
| | | | proteinase | | | | | | | | | | | |
| | | | inhibitor, clade A | | | | | | | | | | | |
| 48415 | SERPINB1 | | serine (or | 1.0 | 1.0 | 1.8 | 1.7 | 1.3 | 1.0 | 1.0 | 1.0 | 1.0 | 1.0 | 4.7 |
| | | | cysteine) | | | | | | | | | | | |
| | | | proteinase | | | | | | | | | | | |
| | | | inhibitor, clade B | | | | | | | | | | | |
| 47876 | SERPINB3 | | serine (or | 4.9 | 4.8 | 1.2 | 1.1 | 1.0 | 1.0 | 1.1 | 1.0 | 0.9 | 1.0 | 4.1 |
| | | | cysteine) | | | | | | | | | | | |
| | | | proteinase | | | | | | | | | | | |
| | | | inhibitor, clade B | | | | | | | | | | | |
| 47332 | SERPINB3 | | serine (or | 4.7 | 4.1 | 1.1 | 1.0 | 1.0 | 1.0 | 1.0 | 1.0 | 1.2 | 1.0 | 4.0 |
| | | | cysteine) | | | | | | | | | | | |
| | | | proteinase | | | | | | | | | | | |
| | | | inhibitor, clade B | | | | | | | | | | | |
| 49963 | SERPINB8 | | serine (or | 3.7 | 3.4 | 1.6 | 1.5 | 1.0 | 1.0 | 1.2 | 1.0 | 1.0 | 1.0 | 4.5 |
| | | | cysteine) | | | | | | | | | | | |
| | | | proteinase | | | | | | | | | | | |
| | | | inhibitor, clade B | | | | | | | | | | | |
| 48205 | SERPINB8 | | serine (or | 3.3 | 2.4 | 1.6 | 1.5 | 1.0 | 1.0 | 1.2 | 1.0 | 1.0 | 1.0 | 4.5 |
| | | | cysteine) | | | | | | | | | | | |
| | | | proteinase | | | | | | | | | | | |
| | | | inhibitor, clade B | | | | | | | | | | | |
| 49991 | SERPINE1 | | serine (or | 2.0 | 1.9 | 1.4 | 1.2 | 1.2 | 1.1 | 0.7 | 1.0 | [symbol] 4 | 1.3 | 4.6 |
| | | | cysteine) | | | | | | | | | | | |
| | | | proteinase | | | | | | | | | | | |
| | | | inhibitor, clade E | | | | | | | | | | | |
| 48269 | SERPINE1 | | serine (or | 1.9 | 1.8 | 1.3 | 1.1 | 1.1 | 1.1 | 0.9 | 1.0 | [symbol] | 1.3 | 4.5 |
| | | | cysteine) | | | | | | | | | | | |
| | | | proteinase | | | | | | | | | | | |
| | | | inhibitor, clade E | | | | | | | | | | | |
| 48400 | SERPINE2 | | serine (or | 3.3 | 2.7 | 1.3 | 1.2 | 1.1 | 1.1 | 1.0 | 1.0 | 1.0 | 1.0 | 4.3 |
| | | | cysteine) | | | | | | | | | | | |
| | | | proteinase | | | | | | | | | | | |
| | | | inhibitor, clade E | | | | | | | | | | | |
| 48367 | SERPINE2 | | serine (or | 3.5 | 2.7 | 1.4 | 1.2 | 1.1 | 1.0 | 1.0 | 1.0 | 1.1 | 1.0 | 4.2 |
| | | | cysteine) | | | | | | | | | | | |
| | | | proteinase | | | | | | | | | | | |
| | | | inhibitor, clade E | | | | | | | | | | | |
| 48281 | SERPINE2 | | serine (or | 3.7 | 2.9 | 1.4 | 1.2 | 1.1 | 1.0 | 1.0 | 1.0 | 1.1 | 1.0 | 4.2 |
| | | | cysteine) | | | | | | | | | | | |
| | | | proteinase | | | | | | | | | | | |
| | | | inhibitor, clade E | | | | | | | | | | | |
| 50577 | SIAT4C | | | 2.0 | 1.8 | 1.0 | 1.0 | 0.9 | 1.0 | 1.2 | 1.1 | 1.0 | 1.0 | 4.1 |
| 47211 | SLC26A2 | SLC26A2 | solute carrier | 0.9 | 1.0 | 1.5 | 1.0 | 1.0 | 1.0 | [symbol] | 1.5 | 1.1 | 1.0 | 4.5 |
| | | | family 26 (sulfate | | | | | | | | | | | |
| | | | transporter), me | | | | | | | | | | | |
| 50378 | SLC26A2 | SLC26A2 | solute carrier | 0.7 | 1.0 | 1.6 | 1.1 | 1.0 | 1.0 | [symbol] | 1.4 | 1.0 | 1.0 | 4.5 |
| | | | family 26 (sulfate | | | | | | | | | | | |
| | | | transporter), me | | | | | | | | | | | |
| 47310 | SLC29A1 | ENT1 | solute carrier | 0.6 | 1.5 | 1.0 | 1.0 | 1.0 | 1.0 | 1.1 | 1.0 | 1.1 | 1.0 | 4.0 |
| | | | family 29 | | | | | | | | | | | |
| | | | (nucleoside | | | | | | | | | | | |
| | | | transporters) | | | | | | | | | | | |
| 50641 | SLC5A1 | | | 0.7 | 1.3 | 1.4 | 1.3 | 1.1 | 1.1 | 1.2 | 1.1 | 1.1 | 1.0 | 4.5 |
| 46678 | SLPI | SLPI | secretory | 3.1 | 1.9 | 0.7 | 1.0 | 0.8 | 1.1 | 0.7 | 1.0 | 0.9 | 1.0 | 4.1 |
| | | | leukocyte | | | | | | | | | | | |
| | | | protease inhibitor | | | | | | | | | | | |
| | | | (antileukop | | | | | | | | | | | |
| 47300 | SLPI | SLPI | secretory | 2.9 | 1.9 | 0.8 | 1.1 | 0.9 | 1.0 | 0.8 | 1.0 | 1.2 | 1.0 | 4.1 |
| | | | leukocyte | | | | | | | | | | | |
| | | | protease inhibitor | | | | | | | | | | | |
| | | | (antileukop | | | | | | | | | | | |
| 48182 | SNAPC1 | | | 2.0 | 1.7 | 0.9 | 1.0 | 0.9 | 1.0 | 0.8 | 1.0 | 0.8 | 1.1 | 4.1 |
| 47418 | SNK | | | 2.3 | 2.0 | 0.8 | 1.1 | 1.0 | 1.0 | 0.8 | 1.1 | 1.1 | 1.0 | 4.2 |
| 50706 | SP110 | IFI41, | interferon-induced | 3.5 | 2.0 | 1.1 | 1.0 | 1.0 | 1.0 | 1.2 | 1.1 | 1.3 | 1.0 | 4.1 |
| | | interferon | protein 41, 30 kD | | | | | | | | | | | |
| | | induced | | | | | | | | | | | | |
| | | protein 41 | | | | | | | | | | | | |
| 50162 | SP110 | IFI41, | interferon-induced | 2.6 | 2.2 | 1.2 | 1.1 | 1.0 | 1.0 | 1.2 | 1.0 | 1.2 | 1.0 | 4.1 |
| | | interferon | protein 41, 30 kD | | | | | | | | | | | |
| | | induced | | | | | | | | | | | | |
| | | protein 41 | | | | | | | | | | | | |
| 49990 | SRC | Src | v-src avian | 1.5 | 1.3 | 1.4 | 1.3 | 1.0 | 1.0 | 1.3 | 1.1 | 0.9 | 1.0 | 4.5 |
| | | | sarcoma | | | | | | | | | | | |
| | | | (Schmidt-Ruppin | | | | | | | | | | | |
| | | | A-2) viral onc | | | | | | | | | | | |
| 49446 | SRC | Src | v-src avian | 1.5 | 1.4 | 1.4 | 1.4 | 0.9 | 1.0 | 1.3 | 1.1 | 0.9 | 1.0 | 4.5 |
| | | | sarcoma | | | | | | | | | | | |
| | | | (Schmidt-Ruppin | | | | | | | | | | | |
| | | | A-2) viral onc | | | | | | | | | | | |
| 50003 | STAT5A | STAT5a | signal transducer | 4.8 | 3.9 | 1.2 | 1.1 | 1.0 | 1.0 | 1.0 | 1.0 | 1.1 | 1.1 | 4.2 |
| | | | and activator of | | | | | | | | | | | |
| | | | transcription 5 | | | | | | | | | | | |
| 48987 | STAT5A | STAT5a | signal transducer | 5.0 | 4.6 | 1.1 | 1.0 | 1.1 | 1.0 | 1.0 | 1.0 | 1.1 | 1.0 | 4.0 |
| | | | and activator of | | | | | | | | | | | |
| | | | transcription 5 | | | | | | | | | | | |
| 47323 | STAT5B | STAT5b | signal transducer | 1.2 | 1.0 | 0.9 | 1.0 | 1.0 | 1.0 | 1.0 | 1.0 | [symbol] | 1.3 | 4.3 |
| | | | and activator of | | | | | | | | | | | |
| | | | transcription 5 | | | | | | | | | | | |
| 46796 | [symbol] | | | 2.8 | 2.3 | 1.8 | 1.3 | 1.0 | 1.0 | [symbol] | 2.2 | 1.2 | 1.0 | 5.6 |
| 48769 | SUV39H1 | | | 1.0 | 1.0 | 0.7 | 1.0 | 0.9 | 1.0 | [symbol] | 1.6 | 1.0 | 1.0 | 4.6 |
| 46754 | TAF1A | | | 1.1 | 1.0 | 0.6 | 1.4 | 0.8 | 1.1 | 0.8 | 1.0 | 1.0 | 1.0 | 4.5 |
| 50071 | TAF1C | | | 1.1 | 1.0 | 0.8 | 1.0 | 0.8 | 1.1 | 0.8 | 1.1 | [symbol] | 1.4 | 4.6 |
| 49372 | TAP1 | | | 2.6 | 2.4 | 1.2 | 1.2 | 1.1 | 1.0 | 1.4 | 1.2 | 1.3 | 1.2 | 4.5 |
| 46975 | TBK1 | TANK binding | TANK-binding | 1.6 | 1.5 | 0.9 | 1.0 | 0.8 | 1.0 | 0.8 | 1.0 | 0.9 | 1.0 | 4.1 |
| | | kinase, NAK | kinase 1 | | | | | | | | | | | |
| 46613 | TCF12 | TCF12 | transcription factor | 1.8 | 1.7 | 1.1 | 1.0 | 1.0 | 1.0 | 1.3 | 1.2 | 1.0 | 1.0 | 4.2 |
| | | | 12 (HTF4, helix- | | | | | | | | | | | |
| | | | loop-helix tr | | | | | | | | | | | |
| 50370 | TCFL5 | TCFL5 | transcription | 0.6 | 1.6 | 1.0 | 1.0 | 0.9 | 1.0 | 1.0 | 1.0 | 1.0 | 1.0 | 4.0 |
| | | | factor-like 5 (basic | | | | | | | | | | | |
| | | | helix-loop-heli | | | | | | | | | | | |
| 46991 | TFPI2 | TFPI2 | tissue factor | 2.1 | 1.9 | 0.8 | 1.3 | 1.0 | 1.0 | [symbol] | 1.3 | 1.0 | 1.0 | 4.6 |
| | | | pathway inhibitor 2 | | | | | | | | | | | |
| 47987 | [symbol] | THBS1 | thrombospondin 1 | 1.0 | 1.0 | 0.6 | 1.4 | 0.9 | 1.0 | [symbol] | 1.6 | 0.8 | 1.1 | 5.1 |
| 46859 | TIEG | TGF beta- | TGFB inducible | 1.9 | 1.6 | 1.3 | 1.3 | 1.1 | 1.0 | 1.1 | 1.0 | 1.0 | 1.0 | 4.3 |
| | | inducible early | early growth | | | | | | | | | | | |
| | | growth | response | | | | | | | | | | | |
| | | response | | | | | | | | | | | | |
| 48457 | TIF1 | | | 0.6 | 1.5 | 0.9 | 1.1 | 0.9 | 1.1 | 0.9 | 1.1 | 0.9 | 1.0 | 4.3 |
| 49818 | TIMP2 | TIMP2 | tissue inhibitor of | 0.8 | 1.0 | 1.4 | 1.1 | 1.3 | 1.2 | [symbol] | 1.5 | 1.1 | 1.0 | 4.8 |
| | | | metalloproteinase 2 | | | | | | | | | | | |
| 49420 | [symbol] | TIMP2 | tissue inhibitor of | 0.9 | 1.0 | 2.0 | 1.4 | 1.2 | 1.0 | [symbol] | 1.5 | 1.1 | 1.0 | 5.0 |
| | | | metalloproteinase 2 | | | | | | | | | | | |
| 49785 | TIMP2 | TIMP2 | tissue inhibitor of | 0.9 | 1.0 | 1.5 | 1.1 | 1.3 | 1.2 | [symbol] | 1.4 | 1.1 | 1.0 | 4.6 |
| | | | metalloproteinase 2 | | | | | | | | | | | |
| 48239 | TLN1 | TLN1 | talin 1 | 1.0 | 1.0 | 1.1 | 1.0 | 0.6 | 1.3 | 0.8 | 1.0 | 0.7 | 1.0 | 4.3 |
| 47918 | TNFAIP1 | TNFAIP1 | tumor necrosis | 1.7 | 1.6 | 1.2 | 1.1 | 1.0 | 1.0 | 1.0 | 1.0 | 1.0 | 1.0 | 4.2 |
| | | | factor, alpha- | | | | | | | | | | | |
| | | | induced protein 1 | | | | | | | | | | | |
| | | | (en | | | | | | | | | | | |
| 47936 | TNFAIP1 | TNFAIP1 | tumor necrosis | 1.7 | 1.6 | 1.1 | 1.1 | 1.0 | 1.0 | 1.0 | 1.0 | 1.0 | 1.0 | 4.1 |
| | | | factor, alpha- | | | | | | | | | | | |
| | | | induced protein 1 | | | | | | | | | | | |
| | | | (en | | | | | | | | | | | |
| 47993 | TNFAIP2 | TNFAIP2 | tumor necrosis | 19.5 | 19.3 | 0.9 | 1.0 | 0.8 | 1.1 | 0.9 | 1.0 | 0.9 | 1.0 | 4.1 |
| | | | factor, alpha- | | | | | | | | | | | |
| | | | induced protein 2 | | | | | | | | | | | |
| 47642 | TNFAIP3 | A20, | tumor necrosis | 35.5 | 34.5 | 1.2 | 1.0 | 1.0 | 1.0 | 0.8 | 1.1 | 1.1 | 1.0 | 4.1 |
| | | TNFAIP2, Zn | factor, alpha- | | | | | | | | | | | |
| | | finger | induced protein 3 | | | | | | | | | | | |
| 48770 | TNFAIP3 | A20, | tumor necrosis | 2.2 | 1.8 | 1.0 | 1.0 | 1.0 | 1.0 | 0.9 | 1.0 | 1.0 | 1.0 | 4.0 |
| | | TNFAIP2, Zn | factor, alpha- | | | | | | | | | | | |
| | | finger | induced protein 3 | | | | | | | | | | | |
| 47155 | TNFAIP6 | TNFAIP6 | tumor necrosis | 11.8 | 8.3 | 0.9 | 1.0 | 1.1 | 1.0 | 0.7 | 1.0 | 0.9 | 1.0 | 4.0 |
| | | | factor, alpha- | | | | | | | | | | | |
| | | | induced protein 6 | | | | | | | | | | | |
| 46803 | TNFAIP6 | TNFAIP6 | tumor necrosis | 10.6 | 7.0 | 1.0 | 1.0 | 1.1 | 1.0 | 0.7 | 1.0 | 0.9 | 1.0 | 4.0 |
| | | | factor, alpha- | | | | | | | | | | | |
| | | | induced protein 6 | | | | | | | | | | | |
| 49143 | TNFRSF10B | DR5 | tumor necrosis | 2.2 | 2.0 | 1.2 | 1.1 | 1.1 | 1.0 | 1.3 | 1.1 | 0.9 | 1.0 | 4.2 |
| | | | factor receptor | | | | | | | | | | | |
| | | | superfamily, | | | | | | | | | | | |
| | | | member | | | | | | | | | | | |
| 49272 | TNFRSF10B | DR5 | tumor necrosis | 2.2 | 2.0 | 1.2 | 1.1 | 1.1 | 1.0 | 1.2 | 1.1 | 1.0 | 1.0 | 4.2 |
| | | | factor receptor | | | | | | | | | | | |
| | | | superfamily, | | | | | | | | | | | |
| | | | member | | | | | | | | | | | |
| 47143 | TNFRSF10C | DcR1 | tumor necrosis | 1.2 | 1.1 | 1.2 | 1.1 | 1.0 | 1.0 | [symbol] 4 | 1.3 | 1.3 | 1.1 | 4.5 |
| | | | factor receptor | | | | | | | | | | | |
| | | | superfamily, | | | | | | | | | | | |
| | | | member | | | | | | | | | | | |
| 50365 | TNFRSF1A | TNFR1 | tumor necrosis | 0.5 | 1.6 | 1.1 | 1.1 | 1.0 | 1.0 | 1.2 | 1.1 | 0.9 | 1.0 | 4.1 |
| | | | factor receptor | | | | | | | | | | | |
| | | | superfamily, | | | | | | | | | | | |
| | | | member | | | | | | | | | | | |
| 47697 | TNFRSF6B | DcR3 | tumor necrosis | 1.1 | 1.1 | 0.6 | 1.3 | 0.9 | 1.0 | [symbol] | 1.5 | 0.9 | 1.0 | 4.8 |
| | | | factor receptor | | | | | | | | | | | |
| | | | superfamily, | | | | | | | | | | | |
| | | | member | | | | | | | | | | | |
| 50524 | TNFRSF6B | DcR3 | tumor necrosis | 1.1 | 1.0 | 0.7 | 1.4 | 0.9 | 1.0 | [symbol] | 1.4 | 0.9 | 1.0 | 4.8 |
| | | | factor receptor | | | | | | | | | | | |
| | | | superfamily, | | | | | | | | | | | |
| | | | member | | | | | | | | | | | |
| 47517 | TNFRSF9 | 4-1BB, CD137 | tumor necrosis | 3.4 | 1.9 | 1.0 | 1.0 | 0.9 | 1.0 | 0.9 | 1.0 | 1.0 | 1.0 | 4.1 |
| | | | factor receptor | | | | | | | | | | | |
| | | | superfamily, | | | | | | | | | | | |
| | | | member | | | | | | | | | | | |
| 49303 | TNFSF12 | TWEAK | tumor necrosis | 0.5 | 1.5 | 1.1 | 1.0 | 1.1 | 1.0 | 1.3 | 1.1 | 1.0 | 1.0 | 4.1 |
| | | | factor (ligand) | | | | | | | | | | | |
| | | | superfamily, | | | | | | | | | | | |
| | | | member | | | | | | | | | | | |
| 48626 | TNNT1 | | | 0.9 | 1.0 | 0.6 | 1.5 | 0.8 | 1.1 | 0.8 | 1.2 | 0.9 | 1.0 | 4.7 |
| 48275 | TRAF2 | TRAF2 | TNF receptor- | 1.5 | 1.5 | 0.9 | 1.0 | 0.9 | 1.0 | 0.9 | 1.0 | 1.0 | 1.0 | 4.1 |
| | | | associated factor 2 | | | | | | | | | | | |
| 48078 | TRIP | TRIP | TRAF interacting | 0.9 | 1.0 | 0.7 | 1.2 | 1.0 | 1.0 | [symbol] | 1.5 | 1.0 | 1.0 | 4.7 |
| | | | protein | | | | | | | | | | | |
| 46947 | TRIP | TRIP | TRAF interacting | 0.8 | 1.0 | 0.6 | 1.3 | 0.9 | 1.0 | [symbol] | 1.5 | 1.0 | 1.0 | 4.8 |
| | | | protein | | | | | | | | | | | |
| 46980 | TRIP | TRIP | TRAF interacting | 0.9 | 1.0 | 0.6 | 1.4 | 0.9 | 1.0 | 0.5 | 1.2 | 1.0 | 1.0 | 4.6 |
| | | | protein | | | | | | | | | | | |
| 49901 | TROAP | | | 1.0 | 1.0 | 0.5 | 1.0 | 0.9 | 1.0 | [symbol] | 1.6 | 0.7 | 1.2 | 4.8 |
| 49402 | TWIST | | | 0.8 | 1.0 | 0.7 | 1.0 | 1.0 | 1.0 | [symbol] 4 | 1.3 | 1.2 | 1.0 | 4.4 |
| 50575 | UBD | | | 8.6 | 3.9 | 1.0 | 1.0 | 1.0 | 1.0 | 0.7 | 1.0 | 0.8 | 1.1 | 4.1 |
| 50592 | VARS2 | | | 1.0 | 1.0 | 0.6 | 1.4 | 1.3 | 1.0 | [symbol] | 1.3 | 1.0 | 1.0 | 4.6 |
| 47298 | VAV3 | Vav3 | vav 3 oncogene | 1.0 | 1.0 | 0.6 | 1.4 | 0.9 | 1.0 | 1.0 | 1.0 | 1.0 | 1.0 | 4.5 |
| 48262 | VAV3 | Vav3 | vav 3 oncogene | 0.2 | 5.1 | 0.8 | 1.0 | 0.9 | 1.0 | 1.0 | 1.0 | 1.0 | 1.0 | 4.0 |
| 49899 | WWOX | | | 1.2 | 1.0 | 1.2 | 1.2 | 1.1 | 1.0 | [symbol] 4 | 1.3 | 1.3 | 1.1 | 4.6 |
| 50466 | [symbol] | tristetrapro-lin | zinc finger protein | 1.8 | 1.4 | 1.8 | 1.3 | 1.1 | 1.1 | [symbol] | 1.7 | 1.1 | 1.0 | 5.1 |
| | | | homologous to | | | | | | | | | | | |
| | | | Zfp-36 in mouse | | | | | | | | | | | |
| 48979 | [symbol] | tristetrapro-lin | zinc finger protein | 1.7 | 1.2 | 2.0 | 1.7 | 1.1 | 1.1 | [symbol] 0 | 1.4 | 1.0 | 1.0 | 5.1 |
| | | | homologous to | | | | | | | | | | | |
| | | | Zfp-36 in mouse | | | | | | | | | | | |
| 47758 | ZNF184 | | | 2.3 | 2.0 | 1.3 | 1.0 | 1.3 | 1.2 | 1.1 | 1.0 | 1.3 | 1.2 | 4.4 |
| 50586 | ZNF263 | | | 1.4 | 1.2 | 0.7 | 1.2 | 0.9 | 1.0 | [symbol] | 1.4 | 0.9 | 1.0 | 4.6 |
| 49723 | ZNF76 | | | 0.9 | 1.1 | 0.7 | 1.4 | 0.9 | 1.0 | [symbol] | 1.3 | 0.9 | 1.0 | 4.6 |

Drug Effects in A549 Cells, sum min fold change >=4.5
Genes in bold - ≧1.5-fold differential expression in response to IL-1β
Genes in italics - ≧1.3-fold differential expression in response to Clari and/or FMA alone
Genes in bold and italics - ≧1.3-fold differential expression in response to IL1 + Clari and/or IL1 + FMA

| | | | | | | | | | | | | | | |
|---|---|---|---|---|---|---|---|---|---|---|---|---|---|---|
| 48998 | FADS1 | | fatty acid | 0.8 | 1.0 | 2.1 | 2.0 | 1.1 | 1.1 | [symbol] | 2.0 | 1.0 | 1.0 | 6.0 |
| | | | desaturase 1 | | | | | | | | | | | |
| 48334 | MMP7 | MMP7 | matrix | 4.3 | 3.9 | 0.4 | 2.3 | 0.9 | 1.1 | [symbol] | 1.6 | 0.9 | 1.0 | 6.0 |
| | | | metalloproteinase | | | | | | | | | | | |
| | | | 7 (matrilysin, | | | | | | | | | | | |
| | | | uterine) | | | | | | | | | | | |
| 46950 | SERPINA3 | | serine (or | 20.4 | 14.0 | 0.6 | 1.6 | 0.9 | 1.1 | [symbol] | 2.0 | 0.9 | 1.0 | 5.7 |
| | | | cysteine) | | | | | | | | | | | |
| | | | proteinase | | | | | | | | | | | |
| | | | inhibitor, clade A | | | | | | | | | | | |
| 49417 | HSPA1B | HSPA1B | heat shock 70 kD | 1.0 | 1.0 | 1.5 | 1.3 | 1.0 | 1.0 | [symbol] | 2.0 | [symbol] | 1.3 | 5.6 |
| | | | protein 1B | | | | | | | | | | | |
| 50140 | ECT2 | ECT2 | epithelial cell | 1.2 | 1.1 | 0.5 | 1.6 | 0.9 | 1.1 | [symbol] | 1.7 | 0.8 | 1.2 | 5.6 |
| | | | transforming | | | | | | | | | | | |
| | | | sequence 2 | | | | | | | | | | | |
| | | | oncogene | | | | | | | | | | | |
| 49064 | HSPA1B | HSPA1B | heat shock 70 kD | 1.0 | 1.0 | 1.5 | 1.3 | 1.0 | 1.0 | [symbol] | 1.9 | [symbol] | 1.3 | 5.6 |
| | | | protein 1B | | | | | | | | | | | |
| 48721 | DAF | CD55 | decay | 4.0 | 2.7 | 2.1 | 2.0 | 1.0 | 1.0 | [symbol] | 1.6 | 1.1 | 1.0 | 5.6 |
| | | | accelerating factor | | | | | | | | | | | |
| | | | for complement | | | | | | | | | | | |
| | | | (CD55, Cr | | | | | | | | | | | |
| 46796 | STC1 | | stanniocalcin 1 | 2.8 | 2.3 | 1.8 | 1.3 | 1.0 | 1.0 | [symbol] | 2.2 | 1.2 | 1.0 | 5.6 |
| 48618 | HSPA1B | HSPA1B | heat shock 70 kD | 1.1 | 1.0 | 1.5 | 1.3 | 1.0 | 1.0 | [symbol] | 1.9 | [symbol] | 1.3 | 5.5 |
| | | | protein 1B | | | | | | | | | | | |
| 46632 | PTGES | PTGES | prostaglandin E | 3.2 | 3.1 | 2.4 | 1.9 | 1.2 | 1.0 | [symbol] | 1.6 | 1.2 | 1.0 | 5.5 |
| | | | synthase | | | | | | | | | | | |
| 48032 | ECT2 | ECT2 | epithelial cell | 1.1 | 1.0 | 0.5 | 1.4 | 0.8 | 1.1 | [symbol] | 1.7 | 0.8 | 1.2 | 5.4 |
| | | | transforming | | | | | | | | | | | |
| | | | sequence 2 | | | | | | | | | | | |
| | | | oncogene | | | | | | | | | | | |
| 47176 | PTGES | PTGES | prostaglandin E | 3.5 | 3.4 | 2.6 | 2.0 | 1.2 | 1.1 | [symbol] 0 | 1.3 | 1.1 | 1.0 | 5.4 |
| | | | synthase | | | | | | | | | | | |
| 46942 | DAF | CD55 | decay | 4.0 | 3.6 | 2.0 | 1.9 | 1.0 | 1.0 | [symbol] | 1.4 | 1.2 | 1.0 | 5.3 |
| | | | accelerating factor | | | | | | | | | | | |
| | | | for complement | | | | | | | | | | | |
| | | | (CD55, Cr | | | | | | | | | | | |
| 47185 | HSPA1B | HSPA1B | heat shock 70 kD | 1.2 | 1.0 | 1.3 | 1.1 | 1.0 | 1.0 | [symbol] | 1.9 | 1.4 | 1.2 | 5.3 |
| | | | protein 1B | | | | | | | | | | | |
| 49498 | SERPINA3 | | serine (or | 11.3 | 9.5 | 0.7 | 1.1 | 0.9 | 1.0 | [symbol] | 2.2 | 0.9 | 1.0 | 5.3 |
| | | | cysteine) | | | | | | | | | | | |
| | | | proteinase | | | | | | | | | | | |
| | | | inhibitor, clade A | | | | | | | | | | | |
| 47907 | ERG | ERG | v-ets avian | 4.7 | 4.1 | 1.0 | 1.0 | 1.0 | 1.0 | 1.3 | 1.2 | [symbol] | 2.0 | 5.3 |
| | | | erythroblastosis | | | | | | | | | | | |
| | | | virus E26 | | | | | | | | | | | |
| | | | oncogene re | | | | | | | | | | | |
| 48470 | MCM5 | | | 0.8 | 1.2 | 0.6 | 1.7 | 1.0 | 1.0 | [symbol] | 1.6 | 1.0 | 1.0 | 5.2 |
| 50084 | IDI1 | | isopentyl | 0.7 | 1.0 | 1.7 | 1.6 | 0.8 | 1.1 | [symbol] | 1.4 | 0.9 | 1.1 | 5.2 |
| | | | diphosphate delta | | | | | | | | | | | |
| | | | isomerase | | | | | | | | | | | |
| 49276 | | | | 1.0 | 1.0 | 1.3 | 1.2 | 0.9 | 1.0 | [symbol] | 1.8 | 1.4 | 1.2 | 5.2 |
| 49816 | NFYB | NFYB | nuclear | 0.9 | 1.0 | 0.6 | 1.4 | 0.7 | 1.2 | 0.8 | 1.1 | [symbol] | 1.4 | 5.2 |
| | | | transcription factor | | | | | | | | | | | |
| | | | Y, beta | | | | | | | | | | | |
| 47032 | CIT | CIT | citron (rho- | 0.7 | 1.1 | 0.6 | 1.5 | 0.8 | 1.1 | [symbol] | 1.4 | 0.8 | 1.2 | 5.2 |
| | | | interacting, | | | | | | | | | | | |
| | | | serine/threonine | | | | | | | | | | | |
| | | | kinase 2 | | | | | | | | | | | |
| 49155 | ARTS-1 | | | 0.7 | 1.3 | 0.6 | 1.4 | 0.9 | 1.0 | [symbol] | 1.8 | 1.0 | 1.0 | 5.2 |
| 49873 | INSIG1 | | | 0.6 | 1.1 | 2.1 | 1.3 | 0.9 | 1.0 | [symbol] | 1.9 | 1.0 | 1.0 | 5.2 |
| 48972 | CIT | CIT | citron (rho- | 0.7 | 1.1 | 0.4 | 2.1 | 0.8 | 1.1 | 1.0 | 1.0 | 1.0 | 1.0 | 5.2 |
| | | | interacting, | | | | | | | | | | | |
| | | | serine/threonine | | | | | | | | | | | |
| | | | kinase 2 | | | | | | | | | | | |
| 48971 | P4HA2 | P4HA2 | procollagen- | 1.1 | 1.0 | 2.1 | 1.4 | 1.0 | 1.0 | [symbol] | 1.6 | 1.2 | 1.1 | 5.2 |
| | | | proline, 2- | | | | | | | | | | | |
| | | | oxoglutarate 4- | | | | | | | | | | | |
| | | | dioxygenase | | | | | | | | | | | |
| 48979 | ZFP36 | tristetraprolin | zinc finger protein | 1.7 | 1.2 | 2.0 | 1.7 | 1.1 | 1.1 | [symbol] 0 | 1.4 | 1.0 | 1.0 | 5.1 |
| | | | homologous to | | | | | | | | | | | |
| | | | Zfp-36 in mouse | | | | | | | | | | | |
| 50564 | CRA | | | 0.6 | 1.5 | 0.6 | 1.4 | 0.7 | 1.3 | [symbol] | 1.3 | 0.7 | 1.2 | 5.1 |
| 49671 | PDEF | PDEF | prostate | 1.0 | 1.0 | 1.5 | 1.2 | 1.0 | 1.0 | [symbol] | 1.9 | 1.0 | 1.0 | 5.1 |
| | | | epithelium-specific | | | | | | | | | | | |
| | | | Ets transcription | | | | | | | | | | | |
| | | | fac | | | | | | | | | | | |
| 49764 | EHF | EHF | ets homologous | 1.2 | 1.2 | 0.6 | 1.4 | 0.8 | 1.0 | [symbol] | 1.6 | 0.9 | 1.0 | 5.1 |
| | | | factor | | | | | | | | | | | |
| 48138 | PLAUR | | urokinase | 2.2 | 2.0 | 1.4 | 1.2 | 1.0 | 1.0 | [symbol] | 1.7 | 1.2 | 1.2 | 5.1 |
| | | | plasminogen | | | | | | | | | | | |
| | | | activator receptor | | | | | | | | | | | |
| 47772 | MTHFD2 | | methylene | 1.4 | 1.4 | 0.7 | 1.4 | 0.9 | 1.0 | [symbol] | 1.7 | 0.8 | 1.0 | 5.1 |
| | | | tetrahydrofolate | | | | | | | | | | | |
| | | | dehydrogenase | | | | | | | | | | | |
| 47987 | THBS1 | THBS1 | thrombospondin 1 | 1.0 | 1.0 | 0.6 | 1.4 | 0.9 | 1.0 | [symbol] | 1.6 | 0.8 | 1.1 | 5.1 |
| 49073 | PLOD | PLOD | procollagen- | 0.8 | 1.2 | 1.7 | 1.6 | 1.0 | 1.0 | [symbol] | 1.5 | 1.0 | 1.0 | 5.1 |
| | | | lysine, 2- | | | | | | | | | | | |
| | | | oxoglutarate 5- | | | | | | | | | | | |
| | | | dioxygenase ( | | | | | | | | | | | |
| 49076 | SARM | KIAA0524 | KIAA0524 protein | 0.8 | 1.1 | 0.7 | 1.3 | 0.7 | 1.4 | 0.8 | 1.0 | [symbol] | 1.4 | 5.1 |
| 50466 | ZFP36 | tristetrapro-lin | zinc finger protein | 1.8 | 1.4 | 1.8 | 1.3 | 1.1 | 1.1 | [symbol] | 1.7 | 1.1 | 1.0 | 5.1 |
| | | | homologous to | | | | | | | | | | | |
| | | | Zfp-36 in mouse | | | | | | | | | | | |
| 49347 | HNRPA3 | | | 0.9 | 1.0 | 0.5 | 1.6 | 1.0 | 1.0 | [symbol] | 1.5 | 1.0 | 1.0 | 5.0 |
| 48227 | NRG1 | neuregulin1, | neuregulin 1 | 1.3 | 1.1 | 0.6 | 1.5 | 0.9 | 1.1 | [symbol] | 1.4 | 0.9 | 1.1 | 5.0 |
| | | neu diff. | | | | | | | | | | | | |
| | | Factor, | | | | | | | | | | | | |
| | | heregulin | | | | | | | | | | | | |
| 50037 | LAMC2 | LAMC2 | laminin, gamma 2 | 1.0 | 1.0 | 2.0 | 1.5 | 1.0 | 1.0 | [symbol] 0 | 1.5 | 1.2 | 1.0 | 5.0 |
| | | | (nicein (100 kD), | | | | | | | | | | | |
| | | | kalinin (105 kD), | | | | | | | | | | | |
| 49783 | NFYB | NFYB | nuclear | 0.9 | 1.0 | 0.6 | 1.5 | 0.8 | 1.1 | 0.7 | 1.1 | [symbol] | 1.4 | 5.0 |
| | | | transcription factor | | | | | | | | | | | |
| | | | Y, beta | | | | | | | | | | | |
| 49147 | | | | 1.0 | 1.0 | 1.3 | 1.1 | 0.9 | 1.0 | [symbol] | 1.8 | 1.4 | 1.2 | 5.0 |
| 49952 | AADAC | | | 0.9 | 1.0 | 0.6 | 1.3 | 0.8 | 1.3 | 0.7 | 1.0 | [symbol] | 1.4 | 5.0 |
| 49981 | BMP6 | BMP6 | bone | 1.4 | 1.1 | 1.5 | 1.4 | 0.9 | 1.0 | [symbol] | 1.5 | 0.8 | 1.1 | 5.0 |
| | | | morphogenetic | | | | | | | | | | | |
| | | | protein 6 | | | | | | | | | | | |
| 49420 | TIMP2 | TIMP2 | tissue inhibitor of | 0.9 | 1.0 | 2.0 | 1.4 | 1.2 | 1.0 | [symbol] | 1.5 | 1.1 | 1.0 | 5.0 |
| | | | metalloproteinase 2 | | | | | | | | | | | |
| 46875 | LOC94105 | | | 1.0 | 1.0 | 2.1 | 1.9 | 1.1 | 1.1 | 1.1 | 1.0 | 1.0 | 1.0 | 5.0 |
| 47995 | PTGER1 | PTGER1 | prostaglandin E | 1.2 | 1.0 | 0.7 | 1.4 | 0.9 | 1.1 | [symbol] | 1.4 | 0.8 | 1.1 | 5.0 |
| | | | receptor 1 | | | | | | | | | | | |
| | | | (subtype EP1), | | | | | | | | | | | |
| | | | 42 kD | | | | | | | | | | | |
| 47704 | PLOD | PLOD | procollagen- | 0.8 | 1.2 | 1.6 | 1.5 | 1.1 | 1.0 | [symbol] | 1.5 | 1.1 | 1.0 | 5.0 |
| | | | lysine, 2- | | | | | | | | | | | |
| | | | oxoglutarate 5- | | | | | | | | | | | |
| | | | dioxygenase ( | | | | | | | | | | | |
| 48579 | PRKCBP1 | PRKCBP1 | protein kinase C | 1.3 | 1.3 | 0.7 | 1.2 | 0.8 | 1.1 | [symbol] | 1.5 | 0.8 | 1.1 | 4.9 |
| | | | binding protein 1 | | | | | | | | | | | |
| 49106 | PLOD | PLOD | procollagen- | 0.8 | 1.1 | 1.7 | 1.5 | 1.0 | 1.0 | [symbol] | 1.4 | 1.0 | 1.0 | 4.9 |
| | | | lysine, 2- | | | | | | | | | | | |
| | | | oxoglutarate 5- | | | | | | | | | | | |
| | | | dioxygenase ( | | | | | | | | | | | |
| 46911 | KIAA0170 | | | 1.0 | 1.0 | 0.6 | 1.5 | 0.9 | 1.0 | [symbol] | 1.4 | 0.9 | 1.0 | 4.9 |
| 50474 | CEBPA | C/EBP alpha | CCAAT/enhancer | 0.9 | 1.0 | 1.5 | 1.4 | 1.1 | 1.0 | [symbol] | 1.5 | 1.0 | 1.0 | 4.9 |
| | | | binding protein | | | | | | | | | | | |
| | | | (C/EBP), alpha | | | | | | | | | | | |
| 49690 | NID | NID | nidogen (enactin) | 0.9 | 1.0 | 1.8 | 1.5 | 1.3 | 1.0 | [symbol] 0 | 1.4 | 1.0 | 1.0 | 4.9 |
| 46856 | IL8 | interleukin-8 | interleukin 8 | 239.5 | 161.1 | 2.2 | 1.9 | 0.8 | 1.0 | 1.0 | 1.0 | 1.1 | 1.0 | 4.9 |
| 50096 | PRSS11 | | | 0.7 | 1.5 | 1.6 | 1.3 | 1.1 | 1.1 | [symbol] | 1.4 | 1.2 | 1.1 | 4.9 |
| 49860 | MSX1 | | | 1.2 | 1.1 | 1.2 | 1.1 | 1.0 | 1.0 | [symbol] | 1.6 | 1.1 | 1.1 | 4.8 |
| 47030 | | | | 0.6 | 1.4 | 0.6 | 1.3 | 0.9 | 1.1 | [symbol] | 1.5 | 0.9 | 1.0 | 4.8 |
| 49118 | CCNA2 | cyclin A2 | cyclin A2 | 0.9 | 1.0 | 0.6 | 1.3 | 0.9 | 1.0 | [symbol] | 1.5 | 0.8 | 1.0 | 4.8 |
| 47691 | BATF | B-ATF | basic leucine | 0.8 | 1.0 | 0.8 | 1.0 | 0.6 | 1.5 | 0.7 | 1.0 | 0.7 | 1.2 | 4.8 |
| | | | zipper | | | | | | | | | | | |
| | | | transcription | | | | | | | | | | | |
| | | | factor, ATF-lik | | | | | | | | | | | |
| 48520 | KIAA0418 | | | 1.2 | 1.0 | 1.4 | 1.3 | 1.0 | 1.0 | [symbol] | 1.5 | 1.2 | 1.0 | 4.8 |
| 49818 | TIMP2 | TIMP2 | tissue inhibitor of | 0.8 | 1.0 | 1.4 | 1.1 | 1.3 | 1.2 | [symbol] | 1.5 | 1.1 | 1.0 | 4.8 |
| | | | metalloproteinase 2 | | | | | | | | | | | |
| 50282 | CSRP1 | | | 2.5 | 1.7 | 1.6 | 1.3 | 1.4 | 1.3 | 1.4 | 1.2 | 1.2 | 1.0 | 4.8 |
| 49901 | TROAP | | | 1.0 | 1.0 | 0.5 | 1.0 | 0.9 | 1.0 | [symbol] | 1.6 | 0.7 | 1.2 | 4.8 |
| 46947 | TRIP | TRIP | TRAF interacting | 0.8 | 1.0 | 0.6 | 1.3 | 0.9 | 1.0 | [symbol] | 1.5 | 1.0 | 1.0 | 4.8 |
| | | | protein | | | | | | | | | | | |
| 49098 | BMP4 | BMP4 | bone | 0.8 | 1.1 | 1.4 | 1.2 | 0.9 | 1.0 | [symbol] | 1.6 | 1.0 | 1.0 | 4.8 |
| | | | morphogenetic | | | | | | | | | | | |
| | | | protein 4 | | | | | | | | | | | |
| 50126 | EREG | epiregulin | epiregulin | 3.8 | 3.6 | 1.3 | 1.1 | 1.4 | 1.2 | [symbol] | 1.5 | 1.1 | 1.0 | 4.8 |
| 50042 | LAMB3 | LAMB3 | laminin, beta 3 | 13.3 | 11.0 | 0.7 | 1.3 | 0.9 | 1.0 | [symbol] | 1.4 | 0.8 | 1.1 | 4.8 |
| | | | (nicein (125 kD), | | | | | | | | | | | |
| | | | kalinin (140 kD), | | | | | | | | | | | |
| 46716 | MPHOSPH9 | | | 0.9 | 1.0 | 0.5 | 1.6 | 1.0 | 1.0 | 0.7 | 1.2 | 0.9 | 1.1 | 4.8 |
| 50568 | KNSL5 | | | 1.1 | 1.0 | 0.5 | 1.2 | 0.8 | 1.1 | [symbol] | 1.4 | 0.8 | 1.1 | 4.8 |
| 47976 | DDB2 | | | 0.6 | 1.5 | 0.7 | 1.3 | 0.9 | 1.0 | [symbol] | 1.5 | 1.0 | 1.0 | 4.8 |
| 49533 | PTPRG | PTPRG | protein tyrosine | 0.9 | 1.0 | 1.5 | 1.3 | 1.1 | 1.0 | [symbol] | 1.5 | 1.0 | 1.0 | 4.8 |
| | | | phosphatase, | | | | | | | | | | | |
| | | | receptor type, G | | | | | | | | | | | |
| 49290 | ARHB | RhoB | ras homolog gene | 1.3 | 1.2 | 1.4 | 1.1 | 1.4 | 1.3 | [symbol] | 1.3 | 1.2 | 1.0 | 4.8 |
| | | | family, member B | | | | | | | | | | | |
| 47831 | HEC | | | 1.1 | 1.0 | 0.6 | 1.4 | 0.9 | 1.0 | [symbol] | 1.3 | 1.0 | 1.0 | 4.8 |
| 49738 | PRKCA | PKC alpha | protein kinase C, | 0.9 | 1.0 | 1.5 | 1.4 | 1.0 | 1.0 | [symbol] | 1.3 | 1.1 | 1.0 | 4.8 |
| | | | alpha | | | | | | | | | | | |
| 50319 | BMP6 | BMP6 | bone | 1.2 | 1.2 | 1.5 | 1.4 | 1.0 | 1.0 | [symbol] 4 | 1.3 | 0.9 | 1.1 | 4.8 |
| | | | morphogenetic | | | | | | | | | | | |
| | | | protein 6 | | | | | | | | | | | |
| 50349 | LOC51295 | ECSIT | ECSIT | 0.9 | 1.1 | 0.6 | 1.4 | 1.1 | 1.0 | [symbol] | 1.4 | 1.1 | 1.0 | 4.8 |
| 50524 | TNFRSF6B | DcR3 | tumor necrosis | 1.1 | 1.0 | 0.7 | 1.4 | 0.9 | 1.0 | [symbol] | 1.4 | 0.9 | 1.0 | 4.8 |
| | | | factor receptor | | | | | | | | | | | |
| | | | superfamily, | | | | | | | | | | | |
| | | | member | | | | | | | | | | | |
| 49773 | CCNA2 | cyclin A2 | cyclin A2 | 1.0 | 1.0 | 0.5 | 1.4 | 0.9 | 1.0 | 0.6 | 1.2 | 0.8 | 1.1 | 4.8 |
| 46670 | DUSP5 | DUSP5 | dual specificity | 2.0 | 2.0 | 1.4 | 1.3 | 1.2 | 1.1 | 1.3 | 1.2 | 1.2 | 1.1 | 4.8 |
| | | | phosphatase 5 | | | | | | | | | | | |
| 49113 | HS3ST3A1 | HS3ST3A1 | heparan sulfate | 2.9 | 2.4 | 1.4 | 1.2 | 1.0 | 1.0 | [symbol] | 1.5 | 1.1 | 1.1 | 4.8 |
| | | | (glucosamine) 3- | | | | | | | | | | | |
| | | | O-sulfotransferase | | | | | | | | | | | |
| 47697 | TNFRSF6B | DcR3 | tumor necrosis | 1.1 | 1.1 | 0.6 | 1.3 | 0.9 | 1.0 | [symbol] | 1.5 | 0.9 | 1.0 | 4.8 |
| | | | factor receptor | | | | | | | | | | | |
| | | | superfamily, | | | | | | | | | | | |
| | | | member | | | | | | | | | | | |
| 49167 | KDELR3 | | | 0.9 | 1.0 | 1.5 | 1.1 | 1.1 | 1.0 | [symbol] | 1.6 | 1.1 | 1.0 | 4.8 |
| 47834 | CYP1A1 | | | 1.0 | 1.0 | 1.5 | 1.4 | 1.2 | 1.2 | 1.3 | 1.2 | 1.2 | 1.0 | 4.8 |
| 49798 | FRAG1 | FRAG1 | FGF receptor | 0.7 | 1.4 | 1.2 | 1.1 | 1.1 | 1.1 | [symbol] 4 | 1.4 | 1.2 | 1.2 | 4.7 |
| | | | activating protein 1 | | | | | | | | | | | |
| 48063 | | | | 0.8 | 1.0 | 0.6 | 1.4 | 0.9 | 1.0 | [symbol] | 1.3 | 0.9 | 1.0 | 4.7 |
| 47792 | | | | 1.5 | 1.4 | 0.6 | 1.4 | 0.9 | 1.0 | [symbol] | 1.4 | 0.9 | 1.0 | 4.7 |
| 48626 | TNNT1 | | | 0.9 | 1.0 | 0.6 | 1.5 | 0.8 | 1.1 | 0.8 | 1.2 | 0.9 | 1.0 | 4.7 |
| 49510 | | | | 1.6 | 1.4 | 1.9 | 1.5 | 1.0 | 1.0 | [symbol] | 1.3 | 1.0 | 1.0 | 4.7 |
| 49572 | CRMP1 | | | 0.7 | 1.3 | 1.4 | 1.1 | 1.1 | 1.0 | [symbol] | 1.5 | 1.3 | 1.2 | 4.7 |
| 49688 | GSK3B | GSK3B | glycogen synthase | 1.5 | 1.4 | 1.4 | 1.4 | 1.0 | 1.0 | [symbol] 4 | 1.3 | 0.9 | 1.0 | 4.7 |
| | | | kinase 3 beta | | | | | | | | | | | |
| 48547 | PLA2G1B | phospholipase | phospholipase A2, | 1.0 | 1.0 | 1.0 | 1.0 | 0.6 | 1.7 | 0.8 | 1.0 | 0.8 | 1.0 | 4.7 |
| | | A2 | group IB | | | | | | | | | | | |
| | | | (pancreas) | | | | | | | | | | | |
| 49322 | JUNB | JunB | jun B proto- | 1.2 | 1.1 | 1.7 | 1.6 | 0.9 | 1.0 | 1.3 | 1.0 | 0.8 | 1.1 | 4.7 |
| | | | oncogene | | | | | | | | | | | |
| 49529 | FGA | | | 0.4 | 2.1 | 1.5 | 1.3 | 0.9 | 1.0 | [symbol] | 1.3 | 0.8 | 1.1 | 4.7 |
| 48673 | LRDD | LRDD | leucine rich repeat | 0.9 | 1.0 | 0.7 | 1.3 | 0.8 | 1.1 | [symbol] | 1.4 | 1.0 | 1.0 | 4.7 |
| | | | and death domain | | | | | | | | | | | |
| | | | containing pr | | | | | | | | | | | |
| 47671 | PLOD | PLOD | procollagen- | 0.8 | 1.2 | 1.6 | 1.4 | 1.0 | 1.0 | [symbol] | 1.3 | 1.0 | 1.0 | 4.7 |
| | | | lysine, 2- | | | | | | | | | | | |
| | | | oxoglutarate 5- | | | | | | | | | | | |
| | | | dioxygenase ( | | | | | | | | | | | |
| 49511 | MATN2 | | | 0.6 | 1.3 | 1.5 | 1.4 | 0.9 | 1.0 | 1.5 | 1.2 | 0.9 | 1.0 | 4.7 |
| 49922 | DHCR24 | | | 0.9 | 1.1 | 1.4 | 1.2 | 1.0 | 1.0 | [symbol] | 1.5 | 1.0 | 1.0 | 4.7 |
| 49903 | DDXL | | | 1.0 | 1.0 | 0.7 | 1.4 | 1.0 | 1.0 | [symbol] | 1.3 | 0.9 | 1.0 | 4.7 |
| 48740 | MAP2K6 | MKK6 | mitogen-activated | 0.6 | 1.4 | 1.5 | 1.5 | 0.9 | 1.1 | 1.3 | 1.2 | 1.0 | 1.0 | 4.7 |
| | | | protein kinase | | | | | | | | | | | |
| | | | kinase 6 | | | | | | | | | | | |
| 46577 | PLA2G4B | phospholipase | phospholipase A2, | 1.2 | 1.1 | 0.6 | 1.5 | 0.9 | 1.0 | 0.7 | 1.2 | 0.9 | 1.0 | 4.7 |
| | | A2 | group IVB | | | | | | | | | | | |
| | | | (cytosolic) | | | | | | | | | | | |
| 48707 | P4HA1 | P4HA1, prolyl- | procollagen- | 1.0 | 1.0 | 1.6 | 1.1 | 0.9 | 1.0 | [symbol] | 1.5 | 0.9 | 1.0 | 4.7 |
| | | 4-hydroxylase | proline, 2- | | | | | | | | | | | |
| | | alpha1 | oxoglutarate 4- | | | | | | | | | | | |
| | | | dioxygenase | | | | | | | | | | | |
| 49163 | LGALS3 | | | 1.0 | 1.0 | 1.3 | 1.2 | 1.2 | 1.1 | [symbol] 4 | 1.3 | 1.2 | 1.1 | 4.7 |
| 50348 | PTPRG | PTPRG | protein tyrosine | 0.9 | 1.0 | 1.7 | 1.6 | 1.3 | 1.1 | 1.0 | 1.0 | 1.0 | 1.0 | 4.7 |
| | | | phosphatase, | | | | | | | | | | | |
| | | | receptor type, G | | | | | | | | | | | |
| 48000 | CTSH | | | 0.7 | 1.3 | 1.4 | 1.3 | 1.1 | 1.0 | [symbol] | 1.4 | 1.2 | 1.0 | 4.7 |
| 48282 | ARHG | RhoG | ras homolog gene | 1.8 | 1.6 | 1.3 | 1.2 | 1.1 | 1.0 | [symbol] | 1.4 | 1.2 | 1.1 | 4.7 |
| | | | family, member G | | | | | | | | | | | |
| | | | (rho G) | | | | | | | | | | | |
| 48094 | HSPA6 | HSPA6 | heat shock 70 kD | 1.0 | 1.0 | 1.1 | 1.0 | 1.0 | 1.0 | [symbol] | 1.6 | 1.2 | 1.0 | 4.7 |
| | | | protein 6 | | | | | | | | | | | |
| | | | (HSP70B′) | | | | | | | | | | | |
| 47708 | EHF | EHF | ets homologous | 1.0 | 1.0 | 1.0 | 1.0 | 1.0 | 1.0 | [symbol] | 1.7 | 1.0 | 1.0 | 4.7 |
| | | | factor | | | | | | | | | | | |
| 48663 | KIAA0089 | | | 0.6 | 1.3 | 1.5 | 1.3 | 1.2 | 1.1 | [symbol] | 1.3 | 1.0 | 1.0 | 4.7 |
| 47698 | PTPRJ | PTPRJ | protein tyrosine | 1.7 | 1.6 | 1.6 | 1.5 | 1.0 | 1.0 | 1.3 | 1.2 | 1.0 | 1.0 | 4.7 |
| | | | phosphatase, | | | | | | | | | | | |
| | | | receptor type, J | | | | | | | | | | | |
| 47712 | NR4A2 | TINUR | nuclear receptor | 1.4 | 1.0 | 1.7 | 1.5 | 0.7 | 1.1 | 1.5 | 1.1 | 1.0 | 1.0 | 4.7 |
| | | | subfamily 4, group | | | | | | | | | | | |
| | | | A, member 2 | | | | | | | | | | | |
| 50395 | PTPRF | PTPRF | protein tyrosine | 0.9 | 1.1 | 1.4 | 1.3 | 1.4 | 1.2 | 1.4 | 1.1 | 1.3 | 1.1 | 4.7 |
| | | | phosphatase, | | | | | | | | | | | |
| | | | receptor type, F | | | | | | | | | | | |
| 48078 | TRIP | TRIP | TRAF interacting | 0.9 | 1.0 | 0.7 | 1.2 | 1.0 | 1.0 | [symbol] | 1.5 | 1.0 | 1.0 | 4.7 |
| | | | protein | | | | | | | | | | | |
| 47935 | P4HA1 | P4HA1, prolyl- | procollagen- | 0.9 | 1.0 | 1.5 | 1.2 | 0.8 | 1.1 | [symbol] | 1.4 | 1.0 | 1.0 | 4.7 |
| | | 4-hydroxylase | proline, 2- | | | | | | | | | | | |
| | | alpha1 | oxoglutarate 4- | | | | | | | | | | | |
| | | | dioxygenase | | | | | | | | | | | |
| 47679 | NR4A2 | TINUR | nuclear receptor | 1.3 | 1.0 | 1.7 | 1.6 | 0.8 | 1.0 | 1.3 | 1.0 | 0.8 | 1.1 | 4.7 |
| | | | subfamily 4, group | | | | | | | | | | | |
| | | | A, member 2 | | | | | | | | | | | |
| 48918 | KIAA0514 | | | 0.8 | 1.2 | 1.4 | 1.3 | 1.1 | 1.0 | [symbol] 4 | 1.3 | 1.1 | 1.0 | 4.7 |
| 48415 | SERPINB1 | | serine (or | 1.0 | 1.0 | 1.8 | 1.7 | 1.3 | 1.0 | 1.0 | 1.0 | 1.0 | 1.0 | 4.7 |
| | | | cysteine) | | | | | | | | | | | |
| | | | proteinase | | | | | | | | | | | |
| | | | inhibitor, clade B | | | | | | | | | | | |
| 46817 | ATF3 | ATF3 | activating | 2.3 | 2.0 | 1.3 | 1.3 | 1.4 | 1.1 | [symbol] | 1.3 | 1.0 | 1.0 | 4.7 |
| | | | transcription factor 3 | | | | | | | | | | | |
| 47516 | BTG1 | | | 1.2 | 1.1 | 1.5 | 1.3 | 1.1 | 1.0 | [symbol] | 1.4 | 1.1 | 1.0 | 4.7 |
| 48739 | | | | 1.7 | 1.4 | 0.8 | 1.0 | 1.1 | 1.0 | [symbol] | 1.7 | 1.0 | 1.0 | 4.7 |
| 48943 | CSF3R | G-CSF | colony stimulating | 0.7 | 1.0 | 0.5 | 1.7 | 1.1 | 1.0 | 1.0 | 1.0 | 1.1 | 1.0 | 4.7 |
| | | receptor | factor 3 receptor | | | | | | | | | | | |
| | | | (granulocyte) | | | | | | | | | | | |
| 50632 | PTPRJ | PTPRJ | protein tyrosine | 1.6 | 1.4 | 1.5 | 1.4 | 1.0 | 1.0 | [symbol] | 1.3 | 1.0 | 1.0 | 4.7 |
| | | | phosphatase, | | | | | | | | | | | |
| | | | receptor type, J | | | | | | | | | | | |
| 49292 | LTB | LT, beta | lymphotoxin beta | 4.6 | 2.7 | 1.0 | 1.0 | 1.0 | 1.0 | [symbol] | 1.6 | 1.3 | 1.0 | 4.7 |
| | | | (TNF superfamily, | | | | | | | | | | | |
| | | | member 3) | | | | | | | | | | | |
| 50607 | ITGB3BP | | | 0.8 | 1.0 | 0.6 | 1.0 | 0.9 | 1.1 | [symbol] | 1.4 | 0.8 | 1.2 | 4.7 |
| 47554 | ERG | ERG | v-ets avian | 1.0 | 1.0 | 1.0 | 1.0 | 0.9 | 1.0 | [symbol] | 1.3 | [symbol] | 1.4 | 4.7 |
| | | | erythroblastosis | | | | | | | | | | | |
| | | | virus E26 | | | | | | | | | | | |
| | | | oncogene re | | | | | | | | | | | |
| 47617 | ARF3 | | | 1.0 | 1.0 | 1.0 | 1.0 | 0.5 | 1.7 | 0.8 | 1.0 | 0.7 | 1.0 | 4.7 |
| 48713 | GPRK7 | MNK2 | G protein-coupled | 0.6 | 1.4 | 1.7 | 1.4 | 1.1 | 1.0 | 1.3 | 1.2 | 1.1 | 1.0 | 4.6 |
| | | | receptor kinase 7 | | | | | | | | | | | |
| 46980 | TRIP | TRIP | TRAF interacting | 0.9 | 1.0 | 0.6 | 1.4 | 0.9 | 1.0 | 0.5 | 1.2 | 1.0 | 1.0 | 4.6 |
| | | | protein | | | | | | | | | | | |
| 49424 | PLAU | urokinase-type | plasminogen | 7.9 | 6.6 | 1.5 | 1.3 | 1.4 | 1.3 | 1.1 | 1.0 | 1.3 | 1.0 | 4.6 |
| | | plasminogen | activator, | | | | | | | | | | | |
| | | activator | urokinase | | | | | | | | | | | |
| 48712 | NFYB | NFYB | nuclear | 0.8 | 1.1 | 0.7 | 1.0 | 0.8 | 1.1 | 0.7 | 1.1 | [symbol] | 1.3 | 4.6 |
| | | | transcription factor | | | | | | | | | | | |
| | | | Y, beta | | | | | | | | | | | |
| 49723 | ZNF76 | | | 0.9 | 1.1 | 0.7 | 1.4 | 0.9 | 1.0 | [symbol] | 1.3 | 0.9 | 1.0 | 4.6 |
| 50592 | VARS2 | | | 1.0 | 1.0 | 0.6 | 1.4 | 1.3 | 1.0 | [symbol] | 1.3 | 1.0 | 1.0 | 4.6 |
| 49991 | SERPINE1 | | serine (or | 2.0 | 1.9 | 1.4 | 1.2 | 1.2 | 1.1 | 0.7 | 1.0 | [symbol] 4 | 1.3 | 4.6 |
| | | | cysteine) | | | | | | | | | | | |
| | | | proteinase | | | | | | | | | | | |
| | | | inhibitor, clade E | | | | | | | | | | | |
| 46818 | PIM2 | Pim-2 | pim-2 oncogene | 3.7 | 1.7 | 1.1 | 1.1 | 0.9 | 1.0 | [symbol] | 1.5 | 1.2 | 1.1 | 4.6 |
| 46776 | MAD2L1 | | | 0.9 | 1.0 | 0.7 | 1.1 | 0.8 | 1.1 | [symbol] | 1.4 | 0.9 | 1.0 | 4.6 |
| 49846 | PTEN | PTEN | phosphatase and | 0.7 | 1.3 | 1.4 | 1.2 | 1.0 | 1.0 | [symbol] 4 | 1.3 | 0.9 | 1.0 | 4.6 |
| | | | tensin homolog | | | | | | | | | | | |
| | | | (mutated in multipl | | | | | | | | | | | |
| 50071 | TAF1C | | | 1.1 | 1.0 | 0.8 | 1.0 | 0.8 | 1.1 | 0.8 | 1.1 | [symbol] | 1.4 | 4.6 |
| 50586 | ZNF263 | | | 1.4 | 1.2 | 0.7 | 1.2 | 0.9 | 1.0 | [symbol] | 1.4 | 0.9 | 1.0 | 4.6 |
| 49315 | COL4A2 | COL4A2 | collagen, type IV, | 1.9 | 1.6 | 1.5 | 1.3 | 1.2 | 1.0 | [symbol] | 1.3 | 1.1 | 1.0 | 4.6 |
| | | | alpha 2 | | | | | | | | | | | |
| 48949 | MMP2 | MMP2, | matrix | 0.8 | 1.0 | 1.9 | 1.6 | 1.4 | 1.0 | 1.0 | 1.0 | 1.0 | 1.0 | 4.6 |
| | | gelatinase A | metalloproteinase | | | | | | | | | | | |
| | | | 2 (gelatinase A, | | | | | | | | | | | |
| | | | 72 kD gel | | | | | | | | | | | |
| 49785 | TIMP2 | TIMP2 | tissue inhibitor of | 0.9 | 1.0 | 1.5 | 1.1 | 1.3 | 1.2 | [symbol] | 1.4 | 1.1 | 1.0 | 4.6 |
| | | | metalloproteinase 2 | | | | | | | | | | | |
| 46778 | HMG2 | | | 1.1 | 1.0 | 0.6 | 1.1 | 0.8 | 1.1 | [symbol] | 1.4 | 0.8 | 1.0 | 4.6 |
| 47183 | OSMR | oncostatin MR | oncostatin M | 2.6 | 2.5 | 1.5 | 1.5 | 1.0 | 1.0 | 1.3 | 1.1 | 1.1 | 1.0 | 4.6 |
| | | | receptor | | | | | | | | | | | |
| 49065 | BMP4 | BMP4 | bone | 0.8 | 1.1 | 1.3 | 1.0 | 0.9 | 1.0 | [symbol] | 1.6 | 1.0 | 1.0 | 4.6 |
| | | | morphogenetic | | | | | | | | | | | |
| | | | protein 4 | | | | | | | | | | | |
| 48748 | LTB | LT, beta | lymphotoxin beta | 1.0 | 1.0 | 1.0 | 1.0 | 1.0 | 1.0 | [symbol] | 1.3 | [symbol] | 1.3 | 4.6 |
| | | | (TNF superfamily, | | | | | | | | | | | |
| | | | member 3) | | | | | | | | | | | |
| 48615 | GADD45B | MyD118 | growth arrest and | 0.4 | 2.1 | 1.2 | 1.0 | 0.6 | 1.3 | [symbol] | 1.3 | 1.0 | 1.0 | 4.6 |
| | | | DNA-damage- | | | | | | | | | | | |
| | | | inducible, beta | | | | | | | | | | | |
| 48545 | P2RY1 | P2RY1 | purinergic | 0.8 | 1.1 | 1.0 | 1.0 | 1.3 | 1.2 | 0.9 | 1.0 | [symbol] | 1.4 | 4.6 |
| | | | receptor P2Y, G- | | | | | | | | | | | |
| | | | protein coupled, 1 | | | | | | | | | | | |
| 47308 | PTPRJ | PTPRJ | protein tyrosine | 1.5 | 1.4 | 1.6 | 1.4 | 1.0 | 1.0 | 1.3 | 1.2 | 1.1 | 1.0 | 4.6 |
| | | | phosphatase, | | | | | | | | | | | |
| | | | receptor type, J | | | | | | | | | | | |
| 48480 | CARS | | | 1.1 | 1.1 | 0.8 | 1.2 | 0.9 | 1.0 | [symbol] | 1.4 | 0.9 | 1.0 | 4.6 |
| 48769 | SUV39H1 | | | 1.0 | 1.0 | 0.7 | 1.0 | 0.9 | 1.0 | [symbol] | 1.6 | 1.0 | 1.0 | 4.6 |
| 47135 | CHKL | | | 1.2 | 1.0 | 0.7 | 1.0 | 0.7 | 1.3 | 0.8 | 1.0 | 0.7 | 1.2 | 4.6 |
| 50441 | SAT | | | 3.3 | 3.2 | 0.7 | 1.4 | 0.7 | 1.2 | 0.8 | 1.0 | 0.8 | 1.0 | 4.6 |
| 49124 | MMP7 | MMP7 | matrix | 3.2 | 3.1 | 0.6 | 1.6 | 0.9 | 1.0 | 0.7 | 1.0 | 1.0 | 1.0 | 4.6 |
| | | | metalloproteinase | | | | | | | | | | | |
| | | | 7 (matrilysin, | | | | | | | | | | | |
| | | | uterine) | | | | | | | | | | | |
| 50625 | PSMC6 | | | 1.3 | 1.1 | 0.6 | 1.4 | 0.9 | 1.0 | 0.8 | 1.0 | 0.8 | 1.1 | 4.6 |
| 46991 | TFPI2 | TFPI2 | tissue factor | 2.1 | 1.9 | 0.8 | 1.3 | 1.0 | 1.0 | [symbol] | 1.3 | 1.0 | 1.0 | 4.6 |
| | | | pathway inhibitor 2 | | | | | | | | | | | |
| 48586 | CD4 | CD4 | CD4 antigen (p55) | 0.9 | 1.0 | 0.9 | 1.0 | 0.6 | 1.4 | 0.8 | 1.0 | 0.7 | 1.2 | 4.6 |
| 48344 | ARHG | RhoG | ras homolog gene | 1.7 | 1.5 | 1.2 | 1.1 | 1.1 | 1.1 | [symbol] 4 | 1.4 | 1.1 | 1.1 | 4.6 |
| | | | family, member G | | | | | | | | | | | |
| | | | (rho G) | | | | | | | | | | | |
| 50025 | LIFR | LIFR | leukemia inhibitory | 1.0 | 1.0 | 1.0 | 1.0 | 1.0 | 1.0 | 1.0 | 1.0 | [symbol] | 1.6 | 4.6 |
| | | | factor receptor | | | | | | | | | | | |
| 49060 | CTGF | CTGF | connective tissue | 0.3 | 2.3 | 0.8 | 1.0 | 1.1 | 1.0 | [symbol] | 1.6 | 0.9 | 1.0 | 4.6 |
| | | | growth factor | | | | | | | | | | | |
| 47089 | ACYP2 | | | 0.9 | 1.0 | 1.6 | 1.3 | 1.1 | 1.0 | [symbol] 3 | 1.3 | 1.0 | 1.0 | 4.6 |
| 49883 | FVT1 | | | 1.1 | 1.0 | 1.4 | 1.2 | 1.1 | 1.0 | [symbol] 4 | 1.3 | 1.0 | 1.0 | 4.6 |
| 49516 | HSPA5 | HSPA5 | heat shock 70 kD | 1.1 | 1.0 | 1.6 | 1.3 | 1.2 | 1.1 | 1.2 | 1.1 | 1.2 | 1.0 | 4.6 |
| | | | protein 5 (glucose- | | | | | | | | | | | |
| | | | regulated prote | | | | | | | | | | | |
| 50013 | IL13RA1 | IL-13Ralpha′ | interleukin 13 | 0.6 | 1.2 | 1.3 | 1.2 | 1.1 | 1.1 | [symbol] 4 | 1.3 | 1.0 | 1.0 | 4.6 |
| | | | receptor, alpha 1 | | | | | | | | | | | |
| 47563 | COL11A1 | COL11A1 | collagen, type XI, | 0.7 | 1.2 | 0.7 | 1.4 | 0.9 | 1.0 | 0.8 | 1.1 | 0.8 | 1.0 | 4.6 |
| | | | alpha 1 | | | | | | | | | | | |
| 49899 | WWOX | | | 1.2 | 1.0 | 1.2 | 1.2 | 1.1 | 1.0 | [symbol] 4 | 1.3 | 1.3 | 1.1 | 4.6 |
| 47201 | NFIA | NFAT 3 | nuclear factor I/A | 0.6 | 1.5 | 1.3 | 1.2 | 1.0 | 1.0 | [symbol] | 1.4 | 1.0 | 1.0 | 4.6 |
| 46986 | AREG | amphiregulin | amphiregulin | 2.4 | 2.2 | 0.9 | 1.1 | 1.2 | 1.1 | [symbol] | 1.4 | 1.1 | 1.0 | 4.6 |
| | | | (schwannoma- | | | | | | | | | | | |
| | | | derived growth | | | | | | | | | | | |
| | | | factor) | | | | | | | | | | | |
| 46988 | EREG | epiregulin | epiregulin | 5.4 | 3.4 | 1.1 | 1.1 | 1.3 | 1.0 | [symbol] | 1.5 | 1.1 | 1.0 | 4.6 |
| 50448 | BIRC3 | cIAP2 | baculoviral IAP | 20.3 | 17.7 | 1.5 | 1.3 | 0.8 | 1.2 | 1.2 | 1.1 | 1.0 | 1.0 | 4.6 |
| | | | repeat-containing 3 | | | | | | | | | | | |
| 47208 | PTPN3 | PTPN3 | protein tyrosine | 0.9 | 1.0 | 1.4 | 1.3 | 1.0 | 1.0 | 1.4 | 1.2 | 1.0 | 1.0 | 4.6 |
| | | | phosphatase, non- | | | | | | | | | | | |
| | | | receptor type 3 | | | | | | | | | | | |
| 47922 | HS3ST3A1 | HS3ST3A1 | heparan sulfate | 1.0 | 1.0 | 1.0 | 1.0 | 1.0 | 1.0 | [symbol] | 1.5 | 1.2 | 1.1 | 4.6 |
| | | | (glucosamine) 3- | | | | | | | | | | | |
| | | | O-sulfotransferase | | | | | | | | | | | |
| 49054 | ITGA2 | integrin, alpha | integrin, alpha 2 | 1.7 | 1.3 | 1.5 | 1.3 | 1.0 | 1.0 | 1.3 | 1.2 | 1.1 | 1.0 | 4.6 |
| | | 2, CD49B, | (CD49B, alpha 2 | | | | | | | | | | | |
| | | VLA-2 | subunit of VLA-2 | | | | | | | | | | | |
| 47969 | MAP3K8 | c-cot | mitogen-activated | 5.2 | 3.7 | 0.8 | 1.1 | 0.7 | 1.4 | 1.1 | 1.0 | 1.0 | 1.0 | 4.6 |
| | | | protein kinase | | | | | | | | | | | |
| | | | kinase kinase 8 | | | | | | | | | | | |
| 47979 | BDKRB1 | BDKRB1 | bradykinin | 3.3 | 3.1 | 1.6 | 1.5 | 1.0 | 1.0 | 1.3 | 1.1 | 0.9 | 1.0 | 4.5 |
| | | | receptor B1 | | | | | | | | | | | |
| 49975 | PIM1 | pim-1 | pim-1 oncogene | 1.5 | 1.4 | 0.8 | 1.2 | 1.0 | 1.0 | [symbol] | 1.3 | 0.9 | 1.0 | 4.5 |
| 47523 | DNAJB1 | DNAJB1 | DnaJ (Hsp40) | 1.0 | 1.0 | 1.0 | 1.0 | 0.9 | 1.0 | [symbol] | 1.4 | 1.2 | 1.1 | 4.5 |
| | | | homolog, | | | | | | | | | | | |
| | | | subfamily B, | | | | | | | | | | | |
| | | | member 1 | | | | | | | | | | | |
| 49046 | NFKBIA | IkB, alpha | nuclear factor of | 9.4 | 6.3 | 1.6 | 1.4 | 0.9 | 1.1 | 1.1 | 1.1 | 1.0 | 1.0 | 4.5 |
| | | | kappa light | | | | | | | | | | | |
| | | | polypeptide gene | | | | | | | | | | | |
| | | | enh | | | | | | | | | | | |
| 46825 | GPRK7 | MNK2 | G protein-coupled | 0.7 | 1.1 | 1.5 | 1.4 | 1.1 | 1.0 | 1.2 | 1.1 | 1.1 | 1.0 | 4.5 |
| | | | receptor kinase 7 | | | | | | | | | | | |
| 49372 | TAP1 | | | 2.6 | 2.4 | 1.2 | 1.2 | 1.1 | 1.0 | 1.4 | 1.2 | 1.3 | 1.2 | 4.5 |
| 47191 | LIFR | LIFR | leukemia inhibitory | 1.0 | 1.0 | 1.4 | 1.3 | 1.1 | 1.0 | 1.1 | 1.0 | 1.2 | 1.2 | 4.5 |
| | | | factor receptor | | | | | | | | | | | |
| 50689 | GNB5 | G protein, beta 5 | guanine | 0.9 | 1.0 | 1.3 | 1.2 | 1.1 | 1.0 | [symbol] | 1.3 | 1.2 | 1.0 | 4.5 |
| | | | nucleotide binding | | | | | | | | | | | |
| | | | protein (G | | | | | | | | | | | |
| | | | protein), be | | | | | | | | | | | |
| 49793 | FRAG1 | FRAG1 | FGF receptor | 0.7 | 1.3 | 1.2 | 1.0 | 1.1 | 1.1 | [symbol] 4 | 1.3 | 1.1 | 1.1 | 4.5 |
| | | | activating protein 1 | | | | | | | | | | | |
| 46982 | AREG | amphiregulin | amphiregulin | 2.5 | 2.3 | 0.9 | 1.0 | 1.3 | 1.1 | [symbol] | 1.4 | 1.2 | 1.0 | 4.5 |
| | | | (schwannoma- | | | | | | | | | | | |
| | | | derived growth | | | | | | | | | | | |
| | | | factor) | | | | | | | | | | | |
| 49549 | ANXA8 | | | 1.2 | 1.1 | 0.7 | 1.2 | 1.0 | 1.0 | [symbol] | 1.3 | 0.9 | 1.0 | 4.5 |
| 49645 | SCYD1 | fractalkine | small inducible | 14.5 | 5.8 | 1.0 | 1.0 | 1.0 | 1.0 | [symbol] | 1.3 | 1.4 | 1.2 | 4.5 |
| | | | cytokine subfamily | | | | | | | | | | | |
| | | | D (Cys-X3-Cys), | | | | | | | | | | | |
| 47762 | KIAA0414 | | | 0.9 | 1.0 | 0.7 | 1.3 | 0.8 | 1.0 | 0.8 | 1.2 | 0.9 | 1.0 | 4.5 |
| 50517 | ARHGAP4 | ARHGAP4 | Rho GTPase | 1.1 | 1.0 | 1.2 | 1.1 | 1.0 | 1.0 | [symbol] | 1.4 | 1.0 | 1.0 | 4.5 |
| | | | activating protein 4 | | | | | | | | | | | |
| 48370 | PDGFA | PDGF, alpha | platelet-derived | 0.9 | 1.0 | 1.1 | 1.0 | 1.2 | 1.0 | [symbol] | 1.4 | 1.3 | 1.1 | 4.5 |
| | | | growth factor | | | | | | | | | | | |
| | | | alpha polypeptide | | | | | | | | | | | |
| 48324 | IL3RA | IL-3R alpha | interleukin 3 | 1.7 | 1.2 | 1.9 | 1.5 | 1.0 | 1.0 | 1.3 | 1.0 | 1.0 | 1.0 | 4.5 |
| | | | receptor, alpha | | | | | | | | | | | |
| | | | (low affinity) | | | | | | | | | | | |
| 49475 | PSA | | | 0.8 | 1.1 | 0.9 | 1.0 | 1.0 | 1.0 | [symbol] | 1.3 | 0.8 | 1.2 | 4.5 |
| 49862 | FUT8 | | | 0.7 | 1.4 | 1.4 | 1.2 | 1.0 | 1.0 | [symbol] 4 | 1.4 | 1.0 | 1.0 | 4.5 |
| 48749 | | | | 1.0 | 1.0 | 1.3 | 1.1 | 1.1 | 1.0 | [symbol] | 1.4 | 1.1 | 1.0 | 4.5 |
| 48616 | PPIH | cyclophilin H | peptidyl prolyl | 0.8 | 1.1 | 0.6 | 1.4 | 0.9 | 1.0 | 0.8 | 1.1 | 1.0 | 1.0 | 4.5 |
| | | | isomerase H | | | | | | | | | | | |
| | | | (cyclophilin H) | | | | | | | | | | | |
| 47775 | KIAA0365 | | | 0.9 | 1.0 | 0.7 | 1.3 | 0.8 | 1.0 | 0.8 | 1.2 | 0.9 | 1.0 | 4.5 |
| 48933 | MMP2 | MMP2, | matrix | 1.0 | 1.0 | 1.3 | 1.0 | 1.2 | 1.0 | [symbol] | 1.3 | 1.3 | 1.2 | 4.5 |
| | | gelatinase A | metalloproteinase | | | | | | | | | | | |
| | | | 2 (gelatinase A, | | | | | | | | | | | |
| | | | 72 kD gel | | | | | | | | | | | |
| 49446 | SRC | Src | v-src avian | 1.5 | 1.4 | 1.4 | 1.4 | 0.9 | 1.0 | 1.3 | 1.1 | 0.9 | 1.0 | 4.5 |
| | | | sarcoma | | | | | | | | | | | |
| | | | (Schmidt-Ruppin | | | | | | | | | | | |
| | | | A-2) viral onc | | | | | | | | | | | |
| 50338 | PIM2 | Pim-2 | pim-2 oncogene | 2.4 | 1.8 | 1.2 | 1.1 | 0.9 | 1.0 | [symbol] | 1.4 | 1.0 | 1.0 | 4.5 |
| 48190 | SCYA20 | MIP-3a | small inducible | 328.2 | 325.5 | 1.6 | 1.5 | 1.0 | 1.0 | 0.9 | 1.0 | 0.8 | 1.0 | 4.5 |
| | | | cytokine subfamily | | | | | | | | | | | |
| | | | A (Cys—Cys), me | | | | | | | | | | | |
| 47665 | PTPRJ | PTPRJ | protein tyrosine | 2.0 | 1.8 | 1.6 | 1.5 | 1.0 | 1.0 | 1.2 | 1.0 | 1.0 | 1.0 | 4.5 |
| | | | phosphatase, | | | | | | | | | | | |
| | | | receptor type, J | | | | | | | | | | | |
| 50118 | MAPKAPK3 | MAPKAPK3 | mitogen-activated | 0.8 | 1.1 | 1.2 | 1.0 | 1.1 | 1.1 | [symbol] | 1.4 | 1.3 | 1.0 | 4.5 |
| | | | protein kinase- | | | | | | | | | | | |
| | | | activated protein | | | | | | | | | | | |
| 47897 | NR4A2 | TINUR | nuclear receptor | 1.6 | 1.1 | 1.9 | 1.4 | 0.9 | 1.0 | 1.5 | 1.1 | 0.9 | 1.0 | 4.5 |
| | | | subfamily 4, group | | | | | | | | | | | |
| | | | A, member 2 | | | | | | | | | | | |
| 50641 | SLC5A1 | | | 0.7 | 1.3 | 1.4 | 1.3 | 1.1 | 1.1 | 1.2 | 1.1 | 1.1 | 1.0 | 4.5 |
| 49284 | MAP2K6 | MKK6 | mitogen-activated | 0.6 | 1.2 | 1.5 | 1.4 | 0.9 | 1.1 | 1.3 | 1.0 | 1.0 | 1.0 | 4.5 |
| | | | protein kinase | | | | | | | | | | | |
| | | | kinase 6 | | | | | | | | | | | |
| 49389 | DUSP1 | MKP-1, | dual specificity | 1.4 | 1.0 | 1.5 | 1.5 | 1.1 | 1.0 | 1.1 | 1.0 | 1.0 | 1.0 | 4.5 |
| | | PTPN10 | phosphatase 1 | | | | | | | | | | | |
| 48162 | KNSL2 | | | 0.9 | 1.0 | 0.7 | 1.2 | 0.9 | 1.0 | [symbol] | 1.3 | 1.0 | 1.0 | 4.5 |
| 48624 | PLA2R1 | phospholipase | phospholipase A2 | 1.0 | 1.0 | 1.4 | 1.0 | 0.9 | 1.0 | [symbol] 0 | 1.5 | 1.0 | 1.0 | 4.5 |
| | | A2 | receptor 1, 180 kD | | | | | | | | | | | |
| 49963 | SERPINB8 | | serine (or | 3.7 | 3.4 | 1.6 | 1.5 | 1.0 | 1.0 | 1.2 | 1.0 | 1.0 | 1.0 | 4.5 |
| | | | cysteine) | | | | | | | | | | | |
| | | | proteinase | | | | | | | | | | | |
| | | | inhibitor, clade B | | | | | | | | | | | |
| 50600 | | | | 1.0 | 1.0 | 0.7 | 1.4 | 1.1 | 1.0 | 1.0 | 1.0 | 1.2 | 1.1 | 4.5 |
| 49085 | CCNA2 | cyclin A2 | cyclin A2 | 0.9 | 1.0 | 0.7 | 1.1 | 1.0 | 1.0 | [symbol] | 1.4 | 0.8 | 1.0 | 4.5 |
| 47298 | VAV3 | Vav3 | vav 3 oncogene | 1.0 | 1.0 | 0.6 | 1.4 | 0.9 | 1.0 | 1.0 | 1.0 | 1.0 | 1.0 | 4.5 |
| 48205 | SERPINB8 | | serine (or | 3.3 | 2.4 | 1.6 | 1.5 | 1.0 | 1.0 | 1.2 | 1.0 | 1.0 | 1.0 | 4.5 |
| | | | cysteine) | | | | | | | | | | | |
| | | | proteinase | | | | | | | | | | | |
| | | | inhibitor, clade B | | | | | | | | | | | |
| 47537 | PLAU | urokinase-type | plasminogen | 9.1 | 7.2 | 1.4 | 1.2 | 1.3 | 1.3 | 1.0 | 1.0 | 1.2 | 1.0 | 4.5 |
| | | plasminogen | activator, | | | | | | | | | | | |
| | | activator | urokinase | | | | | | | | | | | |
| 46609 | | | | 0.9 | 1.0 | 0.6 | 1.4 | 0.8 | 1.0 | 0.8 | 1.1 | 1.0 | 1.0 | 4.5 |
| 46962 | GRB10 | GRB10 | growth factor | 1.5 | 1.4 | 1.6 | 1.5 | 1.1 | 1.0 | 1.2 | 1.0 | 1.1 | 1.0 | 4.5 |
| | | | receptor-bound | | | | | | | | | | | |
| | | | protein 10 | | | | | | | | | | | |
| 48548 | GRAP2 | GRAP2 | GRB2-related | 1.2 | 1.0 | 1.0 | 1.0 | 0.7 | 1.4 | 0.8 | 1.1 | 1.0 | 1.0 | 4.5 |
| | | | adaptor protein 2 | | | | | | | | | | | |
| 46627 | | | | 1.0 | 1.0 | 1.0 | 1.0 | 1.0 | 1.0 | [symbol] | 1.5 | 1.0 | 1.0 | 4.5 |
| 49778 | OSMR | oncostatin MR | oncostatin M | 2.2 | 1.8 | 1.4 | 1.2 | 1.1 | 1.0 | [symbol] | 1.3 | 1.0 | 1.0 | 4.5 |
| | | | receptor | | | | | | | | | | | |
| 47143 | TNFRSF10C | DcR1 | tumor necrosis | 1.2 | 1.1 | 1.2 | 1.1 | 1.0 | 1.0 | [symbol] 4 | 1.3 | 1.3 | 1.1 | 4.5 |
| | | | factor receptor | | | | | | | | | | | |
| | | | superfamily, | | | | | | | | | | | |
| | | | member | | | | | | | | | | | |
| 48969 | FOSL2 | FOSL2, Fra2 | FOS-like antigen 2 | 2.3 | 2.0 | 1.3 | 1.2 | 1.0 | 1.0 | [symbol] 4 | 1.3 | 1.1 | 1.0 | 4.5 |
| 49762 | COL4A4 | COL4A4 | collagen, type IV, | 0.7 | 1.0 | 1.2 | 1.1 | 1.0 | 1.0 | [symbol] | 1.4 | 1.1 | 1.0 | 4.5 |
| | | | alpha 4 | | | | | | | | | | | |
| 48269 | SERPINE1 | | serine (or | 1.9 | 1.8 | 1.3 | 1.1 | 1.1 | 1.1 | 0.9 | 1.0 | [symbol] | 1.3 | 4.5 |
| | | | cysteine) | | | | | | | | | | | |
| | | | proteinase | | | | | | | | | | | |
| | | | inhibitor, clade E | | | | | | | | | | | |
| 50378 | SLC26A2 | SLC26A2 | solute carrier | 0.7 | 1.0 | 1.6 | 1.1 | 1.0 | 1.0 | [symbol] | 1.4 | 1.0 | 1.0 | 4.5 |
| | | | family 26 (sulfate | | | | | | | | | | | |
| | | | transporter), me | | | | | | | | | | | |
| 50313 | PLOD2 | PLOD2 | procollagen- | 1.0 | 1.0 | 1.5 | 1.1 | 0.8 | 1.0 | [symbol] 4 | 1.3 | 1.0 | 1.0 | 4.5 |
| | | | lysine, 2- | | | | | | | | | | | |
| | | | oxoglutarate 5- | | | | | | | | | | | |
| | | | dioxygenase ( | | | | | | | | | | | |
| 50010 | PTGIR | PTGIR | prostaglandin I2 | 0.7 | 1.0 | 0.7 | 1.1 | 1.0 | 1.0 | [symbol] | 1.3 | 0.8 | 1.0 | 4.5 |
| | | | (prostacyclin) | | | | | | | | | | | |
| | | | receptor (IP) | | | | | | | | | | | |
| 49032 | GSK3B | GSK3B | glycogen synthase | 1.4 | 1.2 | 1.4 | 1.3 | 1.0 | 1.0 | 1.4 | 1.2 | 1.1 | 1.0 | 4.5 |
| | | | kinase 3 beta | | | | | | | | | | | |
| 47211 | SLC26A2 | SLC26A2 | solute carrier | 0.9 | 1.0 | 1.5 | 1.0 | 1.0 | 1.0 | [symbol] | 1.5 | 1.1 | 1.0 | 4.5 |
| | | | family 26 (sulfate | | | | | | | | | | | |
| | | | transporter), me | | | | | | | | | | | |
| 50742 | MGEA5 | MGEA5 | meningioma | 0.9 | 1.0 | 1.2 | 1.1 | 1.0 | 1.0 | [symbol] | 1.4 | 0.9 | 1.0 | 4.5 |
| | | | expressed antigen | | | | | | | | | | | |
| | | | 5 (hyaluronidase) | | | | | | | | | | | |
| 46754 | TAF1A | | | 1.1 | 1.0 | 0.6 | 1.4 | 0.8 | 1.1 | 0.8 | 1.0 | 1.0 | 1.0 | 4.5 |
| 50745 | DNAJB1 | DNAJB1 | DnaJ (Hsp40) | 0.9 | 1.0 | 1.0 | 1.0 | 1.0 | 1.0 | [symbol] | 1.4 | 1.1 | 1.0 | 4.5 |
| | | | homolog, | | | | | | | | | | | |
| | | | subfamily B, | | | | | | | | | | | |
| | | | member 1 | | | | | | | | | | | |
| 48679 | BCL3 | Bcl3 | B-cell | 1.0 | 1.0 | 1.1 | 1.0 | 0.6 | 1.5 | 0.9 | 1.0 | 0.8 | 1.0 | 4.5 |
| | | | CLL/lymphoma 3 | | | | | | | | | | | |
| 47705 | MAD4 | MAD4 | Mad4 homolog | 1.0 | 1.0 | 1.5 | 1.1 | 1.0 | 1.0 | [symbol] 0 | 1.4 | 1.1 | 1.0 | 4.5 |
| 49990 | SRC | Src | v-src avian | 1.5 | 1.3 | 1.4 | 1.3 | 1.0 | 1.0 | 1.3 | 1.1 | 0.9 | 1.0 | 4.5 |
| | | | sarcoma | | | | | | | | | | | |
| | | | (Schmidt-Ruppin | | | | | | | | | | | |
| | | | A-2) viral onc | | | | | | | | | | | |
| 48244 | RPS6KA5 | RPS6KA5 | ribosomal protein | 0.8 | 1.1 | 0.8 | 1.1 | 1.0 | 1.0 | [symbol] | 1.3 | 1.0 | 1.0 | 4.5 |
| | | | S6 kinase, 90 kD, | | | | | | | | | | | |
| | | | polypeptide 5 | | | | | | | | | | | |

APPENDIX B

| | | | | | | | | | | | | | | |
|---|---|---|---|---|---|---|---|---|---|---|---|---|---|---|
| | | | | | LPS | | Clari | | FMA | LPS +/− | LPS +/− clari, | LPS +/− | LPS +/− FMA, | |
| | | | | LPS | treated, | Clari | treated, | FMA | treated, | clari, | min | FMA, | min | sum of |
| | | | | treated, | min fold | treated, | min fold | treated, | min fold | wtd | fold | wtd | fold | min |
| | | | | wtd mean | change | wtd mean | change | wtd mean | change | mean | change | mean | change | fold |
| SpotID | GeneSymbol | GeneName | GeneDesc | ratio | (2x) | ratio | (1.5x) | ratio | (1.3x) | ratio | (1.5x) | ratio | (1.3x) | change |

Total Differentials in THP1 Cells
Genes in bold - ≧2-fold differential expression in response to LPS
Genes in italics - ≧1.5-fold differential expression in response to Clari and/or ≧1.3-fold differential expression in response to FMA alone
Genes in bold and italics - ≧1.5-fold differential expression in response to IL1 + Clari and/or ≧1.3-fold differential in response to IL1 + FMA

| | | | | | | | | | | | | | | |
|---|---|---|---|---|---|---|---|---|---|---|---|---|---|---|
| 47030 | | | | 1.3 | 1.2 | 1.9 | 1.7 | 1.1 | 1.1 | 1.5 | 1.3 | [symbol] | 1.4 | 5.4 |
| 48097 | | | | 3.8 | 3.1 | 1.0 | 1.0 | 1.0 | 1.0 | [symbol] | 1.7 | [symbol] | 1.3 | 4.9 |
| 49208 | | | | 0.5 | 1.7 | 0.6 | 1.4 | 0.8 | 1.1 | [symbol] | 1.8 | 0.8 | 1.2 | 5.5 |
| 48074 | | | | 1.4 | 1.1 | 1.8 | 1.6 | 1.1 | 1.1 | [symbol] | 1.8 | 1.1 | 1.1 | 5.6 |
| 49168 | | | | 5.1 | 4.8 | 1.4 | 1.3 | 1.0 | 1.0 | 1.1 | 1.0 | 1.1 | 1.1 | 4.4 |
| 49351 | | | | 0.4 | 2.4 | 1.1 | 1.0 | 1.0 | 1.0 | 1.2 | 1.1 | 0.9 | 1.1 | 4.2 |
| 49411 | | | | 3.1 | 2.2 | 1.2 | 1.0 | 0.9 | 1.1 | 1.4 | 1.2 | 1.2 | 1.0 | 4.4 |
| 49147 | | | | 1.9 | 1.3 | 1.4 | 1.3 | 1.0 | 1.0 | 1.6 | 1.3 | 1.2 | 1.0 | 4.6 |
| 49276 | | | | 1.9 | 1.3 | 1.4 | 1.3 | 1.1 | 1.0 | 1.6 | 1.3 | 1.2 | 1.0 | 4.7 |
| 49838 | | | | 1.0 | 1.0 | 1.9 | 1.7 | 1.1 | 1.0 | [symbol] | 1.9 | 1.1 | 1.0 | 5.7 |
| 49056 | | | | 1.2 | 1.0 | 0.7 | 1.3 | 0.9 | 1.0 | 0.9 | 1.0 | 0.9 | 1.0 | 4.4 |
| 48252 | | | | 3.8 | 3.3 | 0.8 | 1.0 | 0.8 | 1.1 | 1.1 | 1.0 | 1.1 | 1.0 | 4.2 |
| 49594 | | | | 1.4 | 1.4 | 1.5 | 1.3 | 1.0 | 1.0 | [symbol] | 1.5 | 1.1 | 1.0 | 4.9 |
| 48659 | | | | 2.4 | 2.4 | 1.2 | 1.1 | 1.0 | 1.0 | 1.2 | 1.0 | 1.1 | 1.0 | 4.2 |
| 50285 | | | | 0.8 | 1.2 | 1.2 | 1.1 | 1.1 | 1.0 | 1.4 | 1.4 | 1.1 | 1.0 | 4.5 |
| 48364 | | | | 1.3 | 1.0 | 1.3 | 1.1 | 0.9 | 1.0 | 1.4 | 1.3 | 0.9 | 1.0 | 4.5 |
| 50380 | | | | 0.9 | 1.0 | 1.9 | 1.7 | 1.2 | 1.0 | [symbol] | 1.8 | 1.1 | 1.0 | 5.5 |
| 49752 | | | | 2.0 | 1.8 | 0.5 | 2.0 | 0.9 | 1.0 | 0.7 | 1.2 | 1.1 | 1.0 | 5.2 |
| 50199 | | | | 1.1 | 1.0 | 1.9 | 1.8 | 1.0 | 1.0 | 1.5 | 1.2 | 1.0 | 1.0 | 5.0 |
| 48771 | | | | 0.9 | 1.1 | 1.5 | 1.4 | 0.9 | 1.0 | [symbol] | 1.5 | 1.0 | 1.0 | 5.0 |
| 48167 | | | | 2.0 | 1.8 | 1.8 | 1.7 | 1.1 | 1.1 | 1.2 | 1.1 | 1.1 | 1.0 | 4.9 |
| 48957 | | | | 1.0 | 1.0 | 1.4 | 1.3 | 1.0 | 1.0 | [symbol] | 1.5 | 1.1 | 1.0 | 4.8 |
| 49510 | | | | 1.5 | 1.1 | 1.5 | 1.3 | 1.0 | 1.0 | [symbol] | 1.5 | 1.0 | 1.0 | 4.8 |
| 50408 | | | | 1.3 | 1.1 | 0.7 | 1.4 | 1.0 | 1.0 | 0.7 | 1.2 | 1.0 | 1.0 | 4.7 |
| 50728 | | | | 0.6 | 1.7 | 1.4 | 1.3 | 1.1 | 1.1 | 1.3 | 1.2 | 1.0 | 1.0 | 4.6 |
| 49378 | | | | 1.4 | 1.3 | 1.5 | 1.0 | 1.2 | 1.1 | 1.9 | 1.3 | 1.0 | 1.0 | 4.5 |
| 48639 | | | | 0.6 | 1.3 | 1.5 | 1.3 | 1.0 | 1.0 | 1.4 | 1.1 | 0.8 | 1.0 | 4.5 |
| 46578 | | | | 0.7 | 1.2 | 0.6 | 1.5 | 1.0 | 1.0 | 0.7 | 1.0 | 0.8 | 1.0 | 4.5 |
| 46651 | | | | 1.1 | 1.0 | 0.8 | 1.1 | 1.0 | 1.0 | 0.7 | 1.3 | 1.0 | 1.0 | 4.4 |
| 48129 | | | | 1.6 | 1.6 | 0.8 | 1.1 | 1.0 | 1.0 | 0.7 | 1.3 | 1.0 | 1.0 | 4.4 |
| 49491 | | | | 1.8 | 1.3 | 1.1 | 1.0 | 0.9 | 1.0 | 1.5 | 1.4 | 1.2 | 1.0 | 4.4 |
| 48060 | | | | 0.7 | 1.2 | 0.9 | 1.0 | 1.0 | 1.0 | 0.7 | 1.3 | 0.9 | 1.0 | 4.3 |
| 49920 | | | | 1.0 | 1.0 | 0.8 | 1.0 | 0.7 | 1.3 | 0.9 | 1.0 | 0.8 | 1.0 | 4.3 |
| 48183 | | | | 3.3 | 2.9 | 1.4 | 1.3 | 1.0 | 1.0 | 1.0 | 1.0 | 1.1 | 1.0 | 4.3 |
| 48534 | | | | 2.9 | 2.7 | 0.9 | 1.0 | 1.0 | 1.0 | 0.8 | 1.1 | 1.1 | 1.0 | 4.1 |
| 50155 | | | | 0.5 | 2.0 | 0.9 | 1.0 | 1.1 | 1.0 | 1.0 | 1.0 | 1.0 | 1.0 | 4.0 |
| 48120 | | | | 2.6 | 2.1 | 1.0 | 1.0 | 1.0 | 1.0 | 0.9 | 1.0 | 1.0 | 1.0 | 4.0 |
| 50633 | ABL2 | | | 1.8 | 1.3 | 1.1 | 1.0 | 1.0 | 1.0 | 1.6 | 1.4 | 0.9 | 1.0 | 4.4 |
| 50259 | ACE | | | 0.7 | 1.3 | 1.6 | 1.4 | 1.0 | 1.0 | [symbol] | 1.5 | 1.0 | 1.0 | 4.9 |
| 48835 | ACOX1 | | | 1.0 | 1.0 | 1.2 | 1.1 | 1.0 | 1.0 | 1.4 | 1.3 | 1.0 | 1.0 | 4.5 |
| 47896 | ADAM11 | ADAM11 | a disintegrin and | 0.9 | 1.0 | 1.5 | 1.3 | 1.2 | 1.1 | 1.0 | 1.0 | 1.0 | 1.0 | 4.4 |
| | | | metalloproteinase | | | | | | | | | | | |
| | | | domain 11 | | | | | | | | | | | |
| 47352 | ADAM11 | ADAM11 | a disintegrin and | 0.9 | 1.0 | 1.5 | 1.4 | 1.0 | 1.0 | 1.0 | 1.0 | 1.0 | 1.0 | 4.4 |
| | | | metalloproteinase | | | | | | | | | | | |
| | | | domain 11 | | | | | | | | | | | |
| 49421 | ADAM17 | TACE | a disintegrin and | 1.1 | 1.0 | 1.0 | 1.0 | 0.9 | 1.0 | 1.5 | 1.4 | 1.1 | 1.0 | 4.4 |
| | | | metalloproteinase | | | | | | | | | | | |
| | | | domain 17 (tum | | | | | | | | | | | |
| 48442 | ADAM17 | TACE | a disintegrin and | 1.2 | 1.0 | 0.9 | 1.0 | 0.9 | 1.0 | [symbol] | 1.5 | 1.1 | 1.0 | 4.5 |
| | | | metalloproteinase | | | | | | | | | | | |
| | | | domain 17 (tum | | | | | | | | | | | |
| 47217 | ADAMTS1 | ADAMTS1 | a disintegrin-like | 1.9 | 1.6 | 2.3 | 2.0 | 1.1 | 1.0 | 1.5 | 1.4 | 1.1 | 1.0 | 5.4 |
| | | | and | | | | | | | | | | | |
| | | | metalloprotease | | | | | | | | | | | |
| | | | (reprolysin | | | | | | | | | | | |
| 47913 | ADAMTS1 | ADAMTS1 | a disintegrin-like | 0.5 | 1.3 | 1.0 | 1.0 | 0.5 | 1.5 | 1.0 | 1.0 | 0.8 | 1.0 | 4.5 |
| | | | and | | | | | | | | | | | |
| | | | metalloprotease | | | | | | | | | | | |
| | | | (reprolysin | | | | | | | | | | | |
| 48271 | ADCY7 | adenylate | adenylate cyclase 7 | 1.1 | 1.0 | 1.8 | 1.3 | 1.2 | 1.2 | [symbol] | 1.8 | 1.3 | 1.2 | 5.5 |
| | | cyclase | | | | | | | | | | | | |
| 49287 | ADCY7 | adenylate | adenylate cyclase 7 | 1.1 | 1.0 | 1.9 | 1.6 | 1.2 | 1.1 | [symbol] | 1.7 | 1.2 | 1.1 | 5.5 |
| | | cyclase | | | | | | | | | | | | |
| 47329 | ADRB2 | | | 0.8 | 1.1 | 1.5 | 1.4 | 1.0 | 1.0. | 1.3 | 1.1 | 0.9 | 1.0 | 4.5 |
| 50412 | AGPAT1 | | | 0.9 | 1.0 | 1.3 | 1.2 | 1.1 | 1.0 | 1.4 | 1.3 | 1.1 | 1.0 | 4.6 |
| 50345 | AGT | | serine (or | 0.7 | 1.1 | 2.1 | 1.7 | 1.1 | 1.1 | [symbol] | 2.2 | 1.1 | 1.0 | 6.0 |
| | | | cysteine) | | | | | | | | | | | |
| | | | proteinase | | | | | | | | | | | |
| | | | inhibitor, clade A | | | | | | | | | | | |
| 49478 | AIM1 | AIM1 | absent in | 1.1 | 1.0 | 1.5 | 1.4 | 1.3 | 1.2 | 1.1 | 1.0 | 1.2 | 1.0 | 4.5 |
| | | | melanoma 1 | | | | | | | | | | | |
| 49638 | AKT3 | AKT3, PKB | v-akt murine | 16.2 | 14.6 | 1.7 | 1.5 | 1.1 | 1.0 | 1.6 | 1.3 | 1.2 | 1.1 | 4.9 |
| | | | thymoma viral | | | | | | | | | | | |
| | | | oncogene | | | | | | | | | | | |
| | | | homolog 3 (pro | | | | | | | | | | | |
| 47127 | ALAD | | | 25.9 | 15.0 | 1.3 | 1.2 | 1.2 | 1.1 | 1.0 | 1.0 | 1.3 | 1.1 | 4.5 |
| 50435 | AMPD2 | | | 0.9 | 1.0 | 1.3 | 1.2 | 1.0 | 1.0 | 1.5 | 1.4 | 0.9 | 1.0 | 4.6 |
| 48530 | APLP2 | | | 0.9 | 1.1 | 1.7 | 1.7 | 1.0 | 1.0 | [symbol] | 1.6 | 1.0 | 1.0 | 5.3 |
| 46986 | AREG | amphiregulin | amphiregulin | 1.8 | 1.5 | 0.8 | 1.0 | 0.5 | 1.8 | [symbol] | 2.0 | [symbol] | 1.6 | 6.5 |
| | | | (schwannoma- | | | | | | | | | | | |
| | | | derived growth | | | | | | | | | | | |
| | | | factor) | | | | | | | | | | | |
| 46953 | AREG | amphiregulin | amphiregulin | 1.8 | 1.5 | 0.8 | 1.0 | 0.5 | 1.9 | [symbol] | 2.0 | [symbol] | 1.5 | 6.4 |
| | | | (schwannoma- | | | | | | | | | | | |
| | | | derived growth | | | | | | | | | | | |
| | | | factor) | | | | | | | | | | | |
| 46982 | AREG | amphiregulin | amphiregulin | 1.8 | 1.6 | 0.8 | 1.1 | 0.5 | 1.8 | [symbol] | 2.2 | [symbol] | 1.5 | 6.5 |
| | | | (schwannoma- | | | | | | | | | | | |
| | | | derived growth | | | | | | | | | | | |
| | | | factor) | | | | | | | | | | | |
| 46949 | AREG | amphiregulin | amphiregulin | 1.9 | 1.4 | 0.8 | 1.0 | 0.5 | 1.8 | [symbol] | 2.4 | [symbol] | 1.4 | 6.6 |
| | | | (schwannoma- | | | | | | | | | | | |
| | | | derived growth | | | | | | | | | | | |
| | | | factor) | | | | | | | | | | | |
| 48357 | ARF6 | ARF6 | ADP-ribosylation | 1.4 | 1.3 | 1.2 | 1.1 | 1.1 | 1.1 | 1.4 | 1.3 | 1.1 | 1.1 | 4.5 |
| | | | factor 6 | | | | | | | | | | | |
| 50502 | ARF6 | ARF6 | ADP-ribosylation | 1.3 | 1.1 | 1.3 | 1.2 | 1.2 | 1.2 | 1.4 | 1.4 | 1.1 | 1.0 | 4.8 |
| | | | factor 6 | | | | | | | | | | | |
| 50469 | ARF6 | ARF6 | ADP-ribosylation | 1.3 | 1.1 | 1.3 | 1.2 | 1.2 | 1.1 | 1.4 | 1.4 | 1.1 | 1.0 | 4.7 |
| | | | factor 6 | | | | | | | | | | | |
| 50242 | ARHG | RhoG | ras homolog gene | 4.5 | 3.5 | 1.2 | 1.1 | 1.1 | 1.0 | 1.0 | 1.0 | 1.2 | 1.1 | 4.2 |
| | | | family, member G | | | | | | | | | | | |
| | | | (rho G) | | | | | | | | | | | |
| 49317 | ARHGEF9 | ARHGEF9 | Cdc42 guanine | 0.8 | 1.2 | 0.6 | 1.3 | 1.0 | 1.0 | 0.9 | 1.0 | 0.8 | 1.0 | 4.3 |
| | | | exchange factor | | | | | | | | | | | |
| | | | (GEF) 9 | | | | | | | | | | | |
| 47456 | ASB1 | | | 0.5 | 1.6 | 0.6 | 1.5 | 0.9 | 1.0 | 0.7 | 1.2 | 0.9 | 1.0 | 4.7 |
| 46817 | ATF3 | ATF3 | activating | 2.2 | 2.1 | 1.1 | 1.0 | 0.9 | 1.1 | 1.6 | 1.4 | 1.1 | 1.0 | 4.5 |
| | | | transcription factor 3 | | | | | | | | | | | |
| 50635 | B4GALT1 | | | 2.6 | 2.1 | 1.0 | 1.0 | 1.1 | 1.1 | 0.9 | 1.0 | 1.0 | 1.0 | 4.1 |
| 48802 | BAI3 | | | 4.7 | 4.6 | 1.2 | 1.1 | 1.1 | 1.1 | 1.5 | 1.4 | 1.3 | 1.2 | 4.8 |
| 50557 | BCL2 | Bcl2 | B-cell | 1.6 | 1.5 | 0.7 | 1.2 | 1.0 | 1.0 | [symbol] | 1.5 | 1.2 | 1.2 | 4.8 |
| | | | CLL/lymphoma 2 | | | | | | | | | | | |
| 50686 | BCL2 | Bcl2 | B-cell | 1.5 | 1.4 | 0.8 | 1.0 | 1.0 | 1.0 | 1.5 | 1.3 | 1.2 | 1.1 | 4.5 |
| | | | CLL/lymphoma 2 | | | | | | | | | | | |
| 47738 | BCL2A1 | BCL2A1 | BCL2-related | 3.1 | 2.7 | 0.9 | 1.0 | 1.0 | 1.0 | 1.6 | 1.0 | 1.1 | 1.0 | 4.1 |
| | | | protein A1 | | | | | | | | | | | |
| 48697 | BCL3 | Bcl3 | B-cell | 4.1 | 4.0 | 0.6 | 1.5 | 0.8 | 1.2 | 1.1 | 1.0 | 1.1 | 1.0 | 4.8 |
| | | | CLL/lymphoma 3 | | | | | | | | | | | |
| 49927 | BCRP1 | | | 0.8 | 1.2 | 0.8 | 1.2 | 1.0 | 1.0 | 0.7 | 1.3 | 0.9 | 1.0 | 4.5 |
| 47396 | BHLHB2 | | | 2.9 | 2.7 | 1.0 | 1.0 | 1.0 | 1.0 | 1.2 | 1.1 | 1.1 | 1.0 | 4.1 |
| 48408 | BIRC2 | cIAP1 | baculoviral IAP | 3.2 | 3.0 | 1.0 | 1.0 | 0.9 | 1.0 | 0.9 | 1.0 | 1.1 | 1.0 | 4.0 |
| | | | repeat-containing 2 | | | | | | | | | | | |
| 49061 | BIRC2 | cIAP1 | baculoviral IAP | 3.4 | 3.0 | 1.0 | 1.0 | 1.0 | 1.0 | 0.9 | 1.0 | 1.1 | 1.0 | 4.0 |
| | | | repeat-containing 2 | | | | | | | | | | | |
| 48375 | BIRC2 | cIAP1 | baculoviral IAP | 3.3 | 3.3 | 0.9 | 1.0 | 1.0 | 1.0 | 0.9 | 1.0 | 1.1 | 1.0 | 4.0 |
| | | | repeat-containing 2 | | | | | | | | | | | |
| 49094 | BIRC2 | cIAP1 | baculoviral IAP | 3.3 | 3.1 | 1.0 | 1.0 | 1.0 | 1.0 | 0.9 | 1.0 | 1.1 | 1.0 | 4.0 |
| | | | repeat-containing 2 | | | | | | | | | | | |
| 50448 | BIRC3 | cIAP2 | baculoviral IAP | 22.0 | 21.6 | 0.9 | 1.0 | 0.9 | 1.1 | 1.0 | 1.0 | 0.9 | 1.0 | 4.1 |
| | | | repeat-containing 3 | | | | | | | | | | | |
| 47497 | BIRC3 | cIAP2 | baculoviral IAP | 11.3 | 7.4 | 1.0 | 1.0 | 1.0 | 1.0 | 1.0 | 1.0 | 1.0 | 1.0 | 4.0 |
| | | | repeat-containing 3 | | | | | | | | | | | |
| 49871 | BLZF1 | | | 3.5 | 3.3 | 1.2 | 1.0 | 1.0 | 1.0 | 1.1 | 1.0 | 1.1 | 1.0 | 4.0 |
| 49098 | BMP4 | BMP4 | bone | 2.8 | 2.3 | 1.0 | 1.0 | 1.0 | 1.0 | 1.0 | 1.0 | 0.9 | 1.0 | 4.0 |
| | | | morphogenetic | | | | | | | | | | | |
| | | | protein 4 | | | | | | | | | | | |
| 49981 | BMP6 | BMP6 | bone | 11.2 | 6.0 | 0.9 | 1.0 | 0.9 | 1.0 | 1.1 | 1.1 | 1.2 | 1.1 | 4.1 |
| | | | morphogenetic | | | | | | | | | | | |
| | | | protein 6 | | | | | | | | | | | |
| 46586 | C12orf8 | | | 0.9 | 1.0 | 1.7 | 1.3 | 1.2 | 1.1 | [symbol] | 1.8 | 1.2 | 1.1 | 5.2 |
| 48543 | C2 | | | 1.9 | 1.7 | 2.0 | 1.6 | 1.3 | 1.2 | 1.3 | 1.1 | 1.2 | 1.2 | 5.1 |
| 46616 | C2orf3 | GCF, TCF-9 | chromosome 2 | 1.1 | 1.0 | 1.6 | 1.5 | 1.1 | 1.0 | 1.5 | 1.2 | 1.2 | 1.1 | 4.9 |
| | | | open reading | | | | | | | | | | | |
| | | | frame 3 | | | | | | | | | | | |
| 49928 | C6orf5 | | | 3.6 | 3.1 | 1.0 | 1.0 | 1.0 | 1.0 | 0.9 | 1.0 | 1.0 | 1.0 | 4.0 |
| 49034 | CARD4 | NOD1 | caspase | 2.3 | 2.2 | 1.2 | 1.0 | 1.0 | 1.0 | 1.2 | 1.0 | 1.0 | 1.0 | 4.0 |
| | | | recruitment | | | | | | | | | | | |
| | | | domain 4 | | | | | | | | | | | |
| 48267 | CASP1 | caspase 1, | caspase 1, | 5.9 | 4.1 | 2.2 | 1.9 | 1.2 | 1.0 | 1.3 | 1.1 | 1.3 | 1.2 | 5.3 |
| | | ICE | apoptosis-related | | | | | | | | | | | |
| | | | cysteine protease | | | | | | | | | | | |
| | | | (in | | | | | | | | | | | |
| 49132 | CASP1 | caspase 1, | caspase 1, | 4.1 | 3.0 | 1.9 | 1.4 | 1.1 | 1.1 | 1.5 | 1.3 | 1.2 | 1.2 | 5.0 |
| | | ICE | apoptosis-related | | | | | | | | | | | |
| | | | cysteine protease | | | | | | | | | | | |
| | | | (in | | | | | | | | | | | |
| 47007 | CASP4 | CASP4 | caspase 4, | 4.4 | 3.2 | 1.0 | 1.0 | 1.0 | 1.0 | 1.1 | 1.0 | 1.2 | 1.2 | 4.2 |
| | | | apoptosis-related | | | | | | | | | | | |
| | | | cysteine protease | | | | | | | | | | | |
| 49807 | CASP4 | CASP4 | caspase 4, | 3.4 | 3.2 | 1.0 | 1.0 | 1.1 | 1.0 | 1.1 | 1.0 | 1.2 | 1.1 | 4.1 |
| | | | apoptosis-related | | | | | | | | | | | |
| | | | cysteine protease | | | | | | | | | | | |
| 50721 | CASP4 | CASP4 | caspase 4, | 3.3 | 2.8 | 1.0 | 1.0 | 1.1 | 1.0 | 1.0 | 1.0 | 1.0 | 1.0 | 4.0 |
| | | | apoptosis-related | | | | | | | | | | | |
| | | | cysteine protease | | | | | | | | | | | |
| 50188 | CASP4 | CASP4 | caspase 4, | 3.0 | 2.4 | 1.0 | 1.0 | 1.1 | 1.0 | 1.0 | 1.0 | 1.0 | 1.0 | 4.0 |
| | | | apoptosis-related | | | | | | | | | | | |
| | | | cysteine protease | | | | | | | | | | | |
| 48336 | CASP7 | caspase-7 | caspase 7, | 3.2 | 3.1 | 0.8 | 1.0 | 0.9 | 1.0 | 0.8 | 1.1 | 1.1 | 1.0 | 4.1 |
| | | | apoptosis-related | | | | | | | | | | | |
| | | | cysteine protease | | | | | | | | | | | |
| 49425 | CASP7 | caspase-7 | caspase 7, | 3.8 | 3.5 | 0.9 | 1.0 | 1.0 | 1.0 | 0.8 | 1.1 | 1.1 | 1.0 | 4.1 |
| | | | apoptosis-related | | | | | | | | | | | |
| | | | cysteine protease | | | | | | | | | | | |
| 49909 | CASR | | | 3.4 | 2.5 | 1.0 | 1.0 | 0.9 | 1.0 | 1.3 | 1.2 | 1.0 | 1.0 | 4.2 |
| 48117 | CAST | | | 1.2 | 1.1 | 1.5 | 1.4 | 1.1 | 1.1 | 1.5 | 1.3 | 1.2 | 1.1 | 4.9 |
| 49698 | CAT | | | 0.9 | 1.0 | 1.5 | 1.4 | 1.2 | 1.1 | 1.2 | 1.2 | 1.1 | 1.0 | 4.7 |
| 50473 | CBL | Cbl | Cas-Br-M (murine) | 0.5 | 2.2 | 1.2 | 1.1 | 1.0 | 1.0 | 1.1 | 1.0 | 0.9 | 1.0 | 4.1 |
| | | | ecotropic retroviral | | | | | | | | | | | |
| | | | transformin | | | | | | | | | | | |
| 49773 | CCNA2 | cyclin A2 | cyclin A2 | 0.9 | 1.0 | 0.5 | 1.5 | 1.0 | 1.0 | 0.6 | 1.4 | 1.1 | 1.1 | 4.9 |
| 49806 | CCNA2 | cyclin A2 | cyclin A2 | 0.7 | 1.2 | 0.5 | 1.6 | 1.1 | 1.0 | 0.5 | 1.0 | 1.2 | 1.0 | 4.7 |
| 49118 | CCNA2 | cyclin A2 | cyclin A2 | 0.7 | 1.3 | 0.5 | 1.3 | 1.0 | 1.0 | 0.6 | 1.0 | 1.0 | 1.0 | 4.3 |
| 48015 | CCND3 | Cyclin D3 | cyclin D3 | 1.2 | 1.1 | 1.6 | 1.5 | 1.1 | 1.0 | 1.0 | 1.0 | 1.1 | 1.0 | 4.6 |
| 47926 | CCNE2 | cyclin E2 | cyclin E2 | 1.6 | 1.2 | 0.5 | 1.8 | 1.0 | 1.0 | 0.6 | 1.2 | 1.2 | 1.0 | 5.0 |
| 48683 | CCR1 | CCR1 | chemokine (C—C | 1.0 | 1.0 | 1.4 | 1.4 | 1.1 | 1.0 | 1.4 | 1.3 | 0.9 | 1.0 | 4.8 |
| | | | motif) receptor 1 | | | | | | | | | | | |
| 48790 | CCR1 | CCR1 | chemokine (C—C | 1.2 | 1.0 | 1.5 | 1.3 | 1.1 | 1.0 | 1.4 | 1.3 | 0.9 | 1.0 | 4.6 |
| | | | motif) receptor 1 | | | | | | | | | | | |
| 47154 | CCR7 | CCR7 | chemokine (C—C | 41.5 | 39.4 | 1.2 | 1.0 | 1.0 | 1.0 | 1.1 | 1.0 | 1.7 | 1.2 | 4.2 |
| | | | motif) receptor 7 | | | | | | | | | | | |
| 47025 | CCR7 | CCR7 | chemokine (C—C | 49.6 | 47.7 | 1.1 | 1.0 | 1.0 | 1.0 | 1.1 | 1.0 | 1.6 | 1.1 | 4.1 |
| | | | motif) receptor 7 | | | | | | | | | | | |
| 50091 | CCRL2 | CCRL2 | chemokine (C—C | 5.3 | 4.1 | 1.2 | 1.1 | 1.1 | 1.1 | 1.4 | 1.2 | 1.2 | 1.0 | 4.5 |
| | | | motif) receptor-like 2 | | | | | | | | | | | |
| 46930 | CD14 | CD14 | CD14 antigen | 0.6 | 1.2 | 2.4 | 2.0 | 1.1 | 1.1 | 1.2 | 1.0 | 0.8 | 1.0 | 5.1 |
| 47927 | CD14 | CD14 | CD14 antigen | 0.6 | 1.4 | 1.8 | 1.6 | 1.0 | 1.0 | 1.0 | 1.0 | 0.9 | 1.0 | 4.6 |
| 47424 | CD36L1 | | | 0.5 | 2.0 | 1.6 | 1.3 | 1.0 | 1.0 | [symbol] | 1.5 | 1.1 | 1.0 | 4.8 |
| 48460 | CD48 | | | 2.6 | 2.3 | 1.0 | 1.0 | 1.2 | 1.0 | 1.2 | 1.0 | 1.0 | 1.0 | 4.0 |
| 48573 | CD69 | CD69 | CD69 antigen | 2.8 | 2.4 | 1.0 | 1.0 | 1.0 | 1.0 | 1.1 | 1.0 | 0.9 | 1.0 | 4.0 |
| | | | (p60, early T-cell | | | | | | | | | | | |
| | | | activation antigen | | | | | | | | | | | |
| 49410 | CD97 | CD97 | CD97 antigen | 3.4 | 2.6 | 1.5 | 1.4 | 1.1 | 1.1 | 0.9 | 1.1 | 1.2 | 1.0 | 4.6 |
| 50392 | CD97 | CD97 | CD97 antigen | 3.4 | 2.9 | 1.4 | 1.3 | 1.0 | 1.0 | 0.8 | 1.2 | 1.1 | 1.0 | 4.5 |
| 49656 | CDK6 | cyclin | cyclin-dependent | 1.2 | 1.0 | 1.3 | 1.2 | 1.0 | 1.0 | [symbol] | 1.6 | 1.0 | 1.0 | 4.8 |
| | | dependent | kinase 6 | | | | | | | | | | | |
| | | kinase 6 | | | | | | | | | | | | |
| 47521 | CDK6 | cyclin | cyclin-dependent | 1.2 | 1.0 | 1.3 | 1.1 | 1.0 | 1.0 | [symbol] | 1.5 | 1.1 | 1.0 | 4.6 |
| | | dependent | kinase 6 | | | | | | | | | | | |
| | | kinase 6 | | | | | | | | | | | | |
| 50474 | CEBPA | C/EBP alpha | CCAAT/enhancer | 0.2 | 4.4 | 1.6 | 1.4 | 1.1 | 1.0 | 1.4 | 1.3 | 0.9 | 1.0 | 4.7 |
| | | | binding protein | | | | | | | | | | | |
| | | | (C/EBP), alpha | | | | | | | | | | | |
| 47891 | CEBPD | CEBP delta | CCAAT/enhancer | 3.8 | 2.9 | 1.3 | 1.2 | 1.0 | 1.0 | 1.2 | 1.1 | 1.3 | 1.2 | 4.4 |
| | | | binding protein | | | | | | | | | | | |
| | | | (C/EBP), delta | | | | | | | | | | | |
| 46849 | CEBPD | CEBP delta | CCAAT/enhancer | 3.8 | 2.5 | 1.3 | 1.0 | 1.0 | 1.0 | 1.5 | 1.1 | 1.2 | 1.0 | 4.2 |
| | | | binding protein | | | | | | | | | | | |
| | | | (C/EBP), delta | | | | | | | | | | | |
| 48013 | CEP3 | CEP3 | Cdc42 effector | 1.4 | 1.3 | 1.4 | 1.2 | 1.0 | 1.0 | [symbol] | 1.8 | 1.2 | 1.1 | 5.1 |
| | | | protein 3 | | | | | | | | | | | |
| 48493 | CG018 | | | 7.0 | 5.5 | 1.1 | 1.0 | 1.0 | 1.0 | 1.5 | 1.3 | 1.1 | 1.0 | 4.3 |
| 49031 | CKB | | | 3.3 | 3.1 | 1.0 | 1.0 | 1.0 | 1.0 | [symbol] | 1.7 | 1.2 | 1.0 | 4.7 |
| 49434 | CKLF1 | chemokine- | chemokine-like | 0.5 | 2.0 | 1.4 | 1.3 | 1.1 | 1.0 | 1.6 | 1.2 | 1.0 | 1.0 | 4.5 |
| | | like factor 3 | factor, | | | | | | | | | | | |
| | | | alternatively | | | | | | | | | | | |
| | | | spliced | | | | | | | | | | | |
| 48373 | CKLF1 | chemokine- | chemokine-like | 0.5 | 2.0 | 1.4 | 1.2 | 1.0 | 1.0 | 1.6 | 1.2 | 1.0 | 1.0 | 4.4 |
| | | like factor 3 | factor, | | | | | | | | | | | |
| | | | alternatively | | | | | | | | | | | |
| | | | spliced | | | | | | | | | | | |
| 49345 | CLU | | | 1.1 | 1.0 | 1.4 | 1.2 | 1.1 | 1.0 | [symbol] | 1.5 | 1.0 | 1.0 | 4.7 |
| 50087 | COL4A1 | COL4A1 | collagen, type IV, | 1.5 | 1.2 | 0.6 | 1.4 | 1.2 | 1.1 | 1.0 | 1.0 | 1.3 | 1.1 | 4.6 |
| | | | alpha 1 | | | | | | | | | | | |
| 47197 | COL4A2 | COL4A2 | collagen, type IV, | 1.2 | 1.1 | 0.6 | 1.3 | 1.0 | 1.0 | 0.9 | 1.0 | 1.3 | 1.2 | 4.5 |
| | | | alpha 2 | | | | | | | | | | | |
| 49315 | COL4A2 | COL4A2 | collagen, type IV, | 1.2 | 1.0 | 0.7 | 1.5 | 1.1 | 1.0 | 1.0 | 1.0 | 1.2 | 1.0 | 4.5 |
| | | | alpha 2 | | | | | | | | | | | |
| 50705 | COL7A1 | COL7A1 | collagen, type VII, | 1.0 | 1.0 | 0.3 | 2.0 | 0.6 | 1.4 | [symbol] | 2.6 | [symbol] | 1.4 | 7.4 |
| | | | alpha 1 | | | | | | | | | | | |
| | | | (epidermolysis | | | | | | | | | | | |
| | | | bullosa | | | | | | | | | | | |
| 50125 | COL7A1 | COL7A1 | collagen, type VII, | 1.0 | 1.0 | 0.3 | 2.3 | 0.6 | 1.4 | [symbol] | 2.6 | [symbol] | 1.4 | 7.7 |
| | | | alpha 1 | | | | | | | | | | | |
| | | | (epidermolysis | | | | | | | | | | | |
| | | | bullosa | | | | | | | | | | | |
| 47505 | COL9A2 | COL9A2 | collagen, type IX, | 1.0 | 1.0 | 1.8 | 1.5 | 1.2 | 1.1 | [symbol] | 1.5 | 1.1 | 1.1 | 5.2 |
| | | | alpha 2 | | | | | | | | | | | |
| 47857 | COL9A2 | COL9A2 | collagen, type IX, | 1.0 | 1.0 | 1.9 | 1.7 | 1.2 | 1.1 | [symbol] | 1.5 | 1.1 | 1.1 | 5.4 |
| | | | alpha 2 | | | | | | | | | | | |
| 50437 | COMT | | | 0.9 | 1.0 | 1.4 | 1.3 | 1.0 | 1.0 | [symbol] | 1.5 | 1.0 | 1.0 | 4.8 |
| 47070 | CPD | | | 1.7 | 1.6 | 1.5 | 1.4 | 1.1 | 1.0 | [symbol] | 1.6 | 1.1 | 1.1 | 5.0 |
| 50564 | CRA | | | 1.0 | 1.0 | 1.3 | 1.0 | 1.0 | 1.0 | [symbol] | 1.6 | 1.0 | 1.0 | 4.6 |
| 47895 | CRADD | RAIDD | CASP2 and | 1.5 | 1.4 | 0.4 | 2.2 | 0.8 | 1.1 | [symbol] | 3.3 | 0.8 | 1.2 | 7.7 |
| | | | RIPK1 domain | | | | | | | | | | | |
| | | | containing adaptor | | | | | | | | | | | |
| | | | with dea | | | | | | | | | | | |
| 49665 | CRADD | RAIDD | CASP2 and | 1.6 | 1.2 | 0.4 | 1.9 | 0.8 | 1.1 | [symbol] | 3.2 | 0.7 | 1.0 | 7.3 |
| | | | RIPK1 domain | | | | | | | | | | | |
| | | | containing adaptor | | | | | | | | | | | |
| | | | with dea | | | | | | | | | | | |
| 46684 | CREB1 | CREB-1 | cAMP responsive | 2.5 | 2.2 | 0.8 | 1.0 | 0.9 | 1.0 | 1.1 | 1.0 | 1.0 | 1.0 | 4.0 |
| | | | element binding | | | | | | | | | | | |
| | | | protein 1 | | | | | | | | | | | |
| 50652 | CRIP1 | | | 0.8 | 1.2 | 1.5 | 1.3 | 1.1 | 1.1 | 1.4 | 1.1 | 1.1 | 1.1 | 4.6 |
| 49278 | CSF1 | M-CSF, | colony stimulating | 1.1 | 1.0 | 0.7 | 1.2 | 1.0 | 1.0 | 0.7 | 1.3 | 1.1 | 1.0 | 4.6 |
| | | macrophage | factor 1 | | | | | | | | | | | |
| | | colony | (macrophage) | | | | | | | | | | | |
| | | stimulating | | | | | | | | | | | | |
| | | factor | | | | | | | | | | | | |
| 49149 | CSF1 | M-CSF, | colony stimulating | 1.1 | 1.0 | 0.7 | 1.3 | 1.0 | 1.0 | 0.7 | 1.3 | 1.1 | 1.0 | 4.6 |
| | | macrophage | factor 1 | | | | | | | | | | | |
| | | colony | (macrophage) | | | | | | | | | | | |
| | | stimulating | | | | | | | | | | | | |
| | | factor | | | | | | | | | | | | |
| 48596 | CSF2 | G-CSF, | colony stimulating | 11.4 | 10.3 | 0.9 | 1.0 | 0.9 | 1.0 | 0.7 | 1.3 | 1.0 | 1.0 | 4.3 |
| | | granulocyte | factor 2 | | | | | | | | | | | |
| | | colony | (granulocyte- | | | | | | | | | | | |
| | | stimulating | macrophag | | | | | | | | | | | |
| | | factor | | | | | | | | | | | | |
| 48052 | CSF2 | G-CSF, | colony stimulating | 12.7 | 10.0 | 0.9 | 1.0 | 1.0 | 1.0 | 0.8 | 1.3 | 1.0 | 1.0 | 4.3 |
| | | granulocyte | factor 2 | | | | | | | | | | | |
| | | colony | (granulocyte- | | | | | | | | | | | |
| | | stimulating | macrophag | | | | | | | | | | | |
| | | factor | | | | | | | | | | | | |
| 49266 | CSF3 | GM-CSF, | colony stimulating | 4.3 | 4.0 | 1.0 | 1.0 | 1.0 | 1.0 | 0.4 | 1.8 | 0.9 | 1.0 | 4.9 |
| | | granulocyte- | factor 3 | | | | | | | | | | | |
| | | macrophage | (granulocyte) | | | | | | | | | | | |
| | | colony | | | | | | | | | | | | |
| | | stimulating | | | | | | | | | | | | |
| | | factor | | | | | | | | | | | | |
| 49137 | CSF3 | GM-CSF, | colony stimulating | 4.8 | 3.9 | 1.0 | 1.0 | 1.0 | 1.0 | 0.5 | 1.4 | 0.9 | 1.0 | 4.5 |
| | | granulocyte- | factor 3 | | | | | | | | | | | |
| | | macrophage | (granulocyte) | | | | | | | | | | | |
| | | colony | | | | | | | | | | | | |
| | | stimulating | | | | | | | | | | | | |
| | | factor | | | | | | | | | | | | |
| 48943 | CSF3R | G-CSF | colony stimulating | 1.0 | 1.0 | 1.5 | 1.3 | 1.2 | 1.0 | 1.8 | 1.2 | 1.0 | 1.0 | 4.6 |
| | | receptor | factor 3 receptor | | | | | | | | | | | |
| | | | (granulocyte) | | | | | | | | | | | |
| 49060 | CTGF | CTGF | connective tissue | 108.1 | 106.7 | 1.0 | 1.0 | 1.3 | 1.0 | [symbol] | 4.9 | 1.1 | 1.0 | 7.9 |
| | | | growth factor | | | | | | | | | | | |
| 47937 | CTSB | | | 1.0 | 1.0 | 1.6 | 1.3 | 1.2 | 1.1 | 1.5 | 1.4 | 1.1 | 1.1 | 4.9 |
| 47226 | CTSD | cathepsin D | cathepsin D | 0.6 | 1.6 | 1.1 | 1.0 | 1.1 | 1.1 | 1.2 | 1.0 | [symbol] | 1.4 | 4.5 |
| | | | (lysosomal | | | | | | | | | | | |
| | | | aspartyl protease) | | | | | | | | | | | |
| 49651 | CTSD | cathepsin D | cathepsin D | 0.6 | 1.4 | 1.2 | 1.2 | 1.3 | 1.2 | 1.5 | 1.3 | 1.5 | 1.2 | 4.9 |
| | | | (lysosomal | | | | | | | | | | | |
| | | | aspartyl protease) | | | | | | | | | | | |
| 47837 | CTSG | | | 1.2 | 1.0 | 1.5 | 1.3 | 0.9 | 1.0 | 1.5 | 1.3 | 1.0 | 1.0 | 4.7 |
| 48000 | CTSH | | | 0.8 | 1.1 | 1.7 | 1.6 | 1.1 | 1.1 | 1.6 | 1.4 | 1.1 | 1.0 | 5.1 |
| 49052 | CXCR4 | CXCR4 | chemokine (C—X—C | 2.0 | 1.7 | 0.4 | 2.2 | 0.9 | 1.0 | 0.7 | 1.3 | 1.1 | 1.0 | 5.5 |
| | | | motif), receptor 4 | | | | | | | | | | | |
| | | | (fusin) | | | | | | | | | | | |
| 50763 | CYBB | | | 1.1 | 1.0 | 1.5 | 1.3 | 1.2 | 1.1 | 1.1 | 1.1 | 1.2 | 1.1 | 4.5 |
| 49787 | CYC1 | cytochrome c-1 | cytochrome c-1 | 1.0 | 1.0 | 0.8 | 1.2 | 1.0 | 1.0 | 0.7 | 1.4 | 1.0 | 1.0 | 4.5 |
| 49820 | CYC1 | cytochrome c-1 | cytochrome c-1 | 1.0 | 1.0 | 0.8 | 1.2 | 1.0 | 1.0 | 0.7 | 1.3 | 1.0 | 1.0 | 4.5 |
| 47834 | CYP1A1 | | | 0.9 | 1.1 | 1.8 | 1.7 | 1.1 | 1.0 | 1.3 | 1.1 | 1.2 | 1.2 | 4.9 |
| 48650 | DAB2 | | | 0.3 | 2.6 | 1.0 | 1.0 | 1.1 | 1.1 | 0.9 | 1.0 | 0.9 | 1.0 | 4.2 |
| 47335 | DAPP1 | | | 2.4 | 1.8 | 1.5 | 1.2 | 1.0 | 1.0 | [symbol] | 1.5 | 1.2 | 1.1 | 4.8 |
| 49903 | DDXL | | | 1.0 | 1.0 | 0.7 | 1.4 | 0.9 | 1.0 | 0.7 | 1.4 | 0.9 | 1.0 | 4.8 |
| 49162 | DKFZP761N09121 | | | 1.0 | 1.0 | 1.9 | 1.6 | 1.1 | 1.0 | [symbol] | 1.7 | 1.1 | 1.0 | 5.3 |
| 50693 | DKFZp762A227 | | | 1.2 | 1.1 | 1.1 | 1.0 | 1.1 | 1.0 | 1.5 | 1.4 | 1.0 | 1.0 | 4.5 |
| 47553 | DKFZp762A227 | | | 1.2 | 1.1 | 1.0 | 1.0 | 1.0 | 1.0 | 1.4 | 1.4 | 1.0 | 1.0 | 4.4 |
| 48333 | DNMT1 | | | 0.6 | 1.5 | 0.7 | 1.4 | 1.0 | 1.0 | 0.7 | 1.3 | 0.9 | 1.0 | 4.7 |
| 48131 | DSCR1 | | | 0.9 | 1.0 | 0.7 | 1.4 | 0.9 | 1.0 | 0.7 | 1.2 | 0.9 | 1.0 | 4.6 |
| 46877 | DTR | HB-EGF, | diphtheria toxin | 2.4 | 2.0 | 0.5 | 1.4 | 0.7 | 1.1 | [symbol] | 1.6 | [symbol] | 1.4 | 5.5 |
| | | diptheria toxin | receptor (heparin- | | | | | | | | | | | |
| | | receptor | binding epiderm | | | | | | | | | | | |
| 50383 | DTR | HB-EGF, | diphtheria toxin | 1.7 | 1.3 | 0.6 | 1.5 | 0.7 | 1.2 | [symbol] | 1.5 | [symbol] | 1.3 | 5.5 |
| | | diptheria toxin | receptor (heparin- | | | | | | | | | | | |
| | | receptor | binding epiderm | | | | | | | | | | | |
| 49389 | DUSP1 | MKP-1, | dual specificity | 5.5 | 5.4 | 0.6 | 1.6 | 0.9 | 1.1 | 0.7 | 1.3 | 0.8 | 1.2 | 5.1 |
| | | PTPN10 | phosphatase 1 | | | | | | | | | | | |
| 50046 | DUSP1 | MKP-1, | dual specificity | 5.6 | 5.0 | 0.5 | 1.8 | 0.9 | 1.0 | 0.7 | 1.2 | 0.9 | 1.0 | 5.0 |
| | | PTPN10 | phosphatase 1 | | | | | | | | | | | |
| 47279 | DUSP10 | DUSP10 | dual specificity | 1.4 | 1.2 | 0.6 | 1.5 | 1.0 | 1.0 | 0.6 | 1.3 | 1.0 | 1.0 | 4.8 |
| | | | phosphatase 10 | | | | | | | | | | | |
| 50362 | DUSP10 | DUSP10 | dual specificity | 1.2 | 1.0 | 0.7 | 1.5 | 1.1 | 1.0 | 0.7 | 1.2 | 1.0 | 1.0 | 4.7 |
| | | | phosphatase 10 | | | | | | | | | | | |
| 50109 | DUSP4 | DUSP4 | dual specificity | 1.8 | 1.6 | 0.9 | 1.0 | 1.0 | 1.0 | 0.7 | 1.3 | 0.8 | 1.1 | 4.4 |
| | | | phosphatase 4 | | | | | | | | | | | |
| 46575 | DUSP4 | DUSP4 | dual specificity | 2.1 | 2.0 | 0.9 | 1.0 | 1.0 | 1.0 | 0.7 | 1.4 | 0.9 | 1.1 | 4.5 |
| | | | phosphatase 4 | | | | | | | | | | | |
| 46670 | DUSP5 | DUSP5 | dual specificity | 3.1 | 2.6 | 0.9 | 1.0 | 0.9 | 1.0 | 1.3 | 1.2 | 1.1 | 1.1 | 4.3 |
| | | | phosphatase 5 | | | | | | | | | | | |
| 47888 | DUSP7 | DUSP7 | dual specificity | 0.2 | 3.4 | 1.2 | 1.0 | 0.9 | 1.1 | 1.0 | 1.0 | 0.9 | 1.1 | 4.2 |
| | | | phosphatase 7 | | | | | | | | | | | |
| 47344 | DUSP7 | DUSP7 | dual specificity | 0.2 | 3.4 | 1.2 | 1.1 | 0.9 | 1.0 | 1.1 | 1.0 | 0.9 | 1.1 | 4.2 |
| | | | phosphatase 7 | | | | | | | | | | | |
| 46619 | DUSP9 | DUSP9 | dual specificity | 2.3 | 2.0 | 1.0 | 1.0 | 1.0 | 1.0 | 1.0 | 1.0 | 1.0 | 1.0 | 4.0 |
| | | | phosphatase 9 | | | | | | | | | | | |
| 46804 | EBI3 | | | 6.2 | 5.8 | 0.9 | 1.1 | 0.9 | 1.0 | 1.0 | 1.0 | 1.1 | 1.0 | 4.1 |
| 47626 | ECE1 | ECE1 | endothelin | 6.3 | 5.7 | 0.8 | 1.0 | 0.8 | 1.0 | 0.8 | 1.2 | 0.8 | 1.1 | 4.3 |
| | | | converting | | | | | | | | | | | |
| | | | enzyme 1 | | | | | | | | | | | |
| 47209 | ECE1 | ECE1 | endothelin | 3.8 | 3.4 | 0.9 | 1.0 | 1.0 | 1.0 | 0.8 | 1.1 | 0.9 | 1.0 | 4.1 |
| | | | converting | | | | | | | | | | | |
| | | | enzyme 1 | | | | | | | | | | | |
| 48911 | ECM1 | ECM1 | extracellular | 0.9 | 1.0 | 1.9 | 1.7 | 1.1 | 1.0 | [symbol] | 1.8 | 1.2 | 1.1 | 5.7 |
| | | | matrix protein 1 | | | | | | | | | | | |
| 50669 | ECM1 | ECM1 | extracellular | 0.9 | 1.0 | 2.0 | 1.8 | 1.1 | 1.0 | [symbol] | 1.9 | 1.2 | 1.1 | 5.8 |
| | | | matrix protein 1 | | | | | | | | | | | |
| 46841 | EDG1 | EDG-1 | endothelial | 3.1 | 2.8 | 0.4 | 1.1 | 1.2 | 1.1 | [symbol] | 1.6 | 1.1 | 1.0 | 4.8 |
| | | | differentiation, | | | | | | | | | | | |
| | | | sphingolipid G- | | | | | | | | | | | |
| | | | protei | | | | | | | | | | | |
| 47152 | EDN1 | EDN1 | endothelin 1 | 1.4 | 1.3 | 1.7 | 1.7 | 1.2 | 1.1 | 0.9 | 1.1 | 1.1 | 1.1 | 4.9 |
| 47023 | EDN1 | EDN1 | endothelin 1 | 1.4 | 1.3 | 1.7 | 1.5 | 1.2 | 1.1 | 0.8 | 1.1 | 1.1 | 1.0 | 4.7 |
| 47675 | EHF | EHF | ets homologous | 3.3 | 2.8 | 1.0 | 1.0 | 1.0 | 1.0 | 0.7 | 1.0 | 1.2 | 1.0 | 4.0 |
| | | | factor | | | | | | | | | | | |
| 47708 | EHF | EHF | ets homologous | 4.1 | 3.5 | 1.0 | 1.0 | 1.0 | 1.0 | 1.0 | 1.0 | 1.1 | 1.0 | 4.0 |
| | | | factor | | | | | | | | | | | |
| 48728 | EIF4EBP1 | PHAS-1 | eukaryotic | 0.4 | 2.1 | 0.8 | 1.2 | 1.1 | 1.0 | 0.7 | 1.4 | 1.0 | 1.0 | 4.5 |
| | | | translation | | | | | | | | | | | |
| | | | initiation factor 4E | | | | | | | | | | | |
| | | | bindin | | | | | | | | | | | |
| 47530 | ELK1 | Elk-1 | ELK1, member of | 3.3 | 2.8 | 1.6 | 1.5 | 1.0 | 1.0 | 1.3 | 1.1 | 1.1 | 1.0 | 4.6 |
| | | | ETS oncogene | | | | | | | | | | | |
| | | | family | | | | | | | | | | | |
| 50170 | EMILIN | EMILIN | elastin microfibril | 0.8 | 1.1 | 1.4 | 1.4 | 1.1 | 1.1 | [symbol] | 1.6 | 1.2 | 1.2 | 5.2 |
| | | | interface located | | | | | | | | | | | |
| | | | protein | | | | | | | | | | | |
| 50714 | EMILIN | EMILIN | elastin microfibril | 0.8 | 1.1 | 1.3 | 1.2 | 1.1 | 1.0 | [symbol] | 1.5 | 1.3 | 1.0 | 4.7 |
| | | | interface located | | | | | | | | | | | |
| | | | protein | | | | | | | | | | | |
| 47580 | EMS1 | cortactin | ems1 sequence | 0.8 | 1.1 | 0.6 | 1.5 | 1.0 | 1.0 | [symbol] | 1.6 | 1.0 | 1.0 | 5.1 |
| | | | (mammary tumor | | | | | | | | | | | |
| | | | and squamous | | | | | | | | | | | |
| | | | cell car | | | | | | | | | | | |
| 48587 | EMS1 | cortactin | ems1 sequence | 0.9 | 1.0 | 0.6 | 1.4 | 1.0 | 1.0 | 0.7 | 1.4 | 1.0 | 1.0 | 4.8 |
| | | | (mammary tumor | | | | | | | | | | | |
| | | | and squamous | | | | | | | | | | | |
| | | | cell car | | | | | | | | | | | |
| 49392 | EPHA2 | | | 1.6 | 1.4 | 0.7 | 1.4 | 1.1 | 1.0 | 0.7 | 1.3 | 1.0 | 1.0 | 4.7 |
| 46988 | EREG | epiregulin | epiregulin | 4.5 | 3.9 | 1.0 | 1.0 | 1.0 | 1.0 | [symbol] | 2.2 | 0.8 | 1.1 | 5.3 |
| 50126 | EREG | epiregulin | epiregulin | 6.6 | 6.2 | 1.0 | 1.0 | 0.7 | 1.0 | [symbol] | 1.9 | 0.8 | 1.1 | 5.0 |
| 46955 | EREG | epiregulin | epiregulin | 3.8 | 3.5 | 1.0 | 1.0 | 1.0 | 1.0 | [symbol] | 2.1 | 0.8 | 1.0 | 5.1 |
| 47907 | ERG | ERG | v-ets avian | 27.1 | 10.3 | 2.7 | 1.7 | 1.2 | 1.0 | 1.3 | 1.0 | 1.2 | 1.1 | 4.9 |
| | | | erythroblastosis | | | | | | | | | | | |
| | | | virus E26 | | | | | | | | | | | |
| | | | oncogene re | | | | | | | | | | | |
| 47554 | ERG | ERG | v-ets avian | 12.8 | 12.2 | 2.5 | 1.7 | 1.2 | 1.0 | 1.2 | 1.0 | 1.1 | 1.0 | 4.8 |
| | | | erythroblastosis | | | | | | | | | | | |
| | | | virus E26 | | | | | | | | | | | |
| | | | oncogene re | | | | | | | | | | | |
| 49672 | ETS2 | Ets-2 | v-ets avian | 6.4 | 4.8 | 0.9 | 1.0 | 0.9 | 1.0 | 0.9 | 1.0 | 1.1 | 1.0 | 4.0 |
| | | | erythroblastosis | | | | | | | | | | | |
| | | | virus E26 | | | | | | | | | | | |
| | | | oncogene ho | | | | | | | | | | | |
| 49674 | ETS2 | Ets-2 | v-ets avian | 7.6 | 2.9 | 1.1 | 1.0 | 0.9 | 1.0 | 0.9 | 1.0 | 1.1 | 1.0 | 4.0 |
| | | | erythroblastosis | | | | | | | | | | | |
| | | | virus E26 | | | | | | | | | | | |
| | | | oncogene ho | | | | | | | | | | | |
| 50382 | ETV4 | ETV4 | ets variant gene 4 | 0.3 | 3.0 | 1.5 | 1.3 | 1.1 | 1.0 | 1.0 | 1.0 | 1.0 | 1.0 | 4.3 |
| | | | (E1A enhancer- | | | | | | | | | | | |
| | | | binding protein, | | | | | | | | | | | |
| 46951 | ETV5 | ETV5 | ets variant gene 5 | 0.3 | 2.9 | 1.6 | 1.5 | 1.1 | 1.1 | 1.4 | 1.0 | 0.9 | 1.0 | 4.6 |
| | | | (ets-related | | | | | | | | | | | |
| | | | molecule) | | | | | | | | | | | |
| 50540 | ETV5 | ETV5 | ets variant gene 5 | 0.8 | 1.0 | 1.5 | 1.4 | 1.1 | 1.0 | 1.3 | 1.2 | 1.0 | 1.0 | 4.5 |
| | | | (ets-related | | | | | | | | | | | |
| | | | molecule) | | | | | | | | | | | |
| 46984 | ETV5 | ETV5 | ets variant gene 5 | 0.3 | 3.7 | 1.7 | 1.4 | 1.1 | 1.0 | 1.6 | 1.0 | 0.9 | 1.0 | 4.4 |
| | | | (ets-related | | | | | | | | | | | |
| | | | molecule) | | | | | | | | | | | |
| 47539 | F2 | F2 | coagulation factor | 1.7 | 1.5 | 0.7 | 1.2 | 0.8 | 1.1 | [symbol] | 2.1 | 0.8 | 1.2 | 5.6 |
| | | | II (thrombin) | | | | | | | | | | | |
| 50758 | F8A | | | 2.1 | 1.7 | 1.2 | 1.1 | 1.1 | 1.0 | 1.4 | 1.3 | 1.2 | 1.0 | 4.5 |
| 49295 | FAF1 | CGI-03 | Fas (TNFRSF6) | 0.9 | 1.0 | 1.5 | 1.4 | 1.1 | 1.0 | 1.4 | 1.4 | 1.2 | 1.2 | 4.9 |
| | | | associated factor 1 | | | | | | | | | | | |
| 47756 | FAP | | | 1.2 | 1.1 | 0.6 | 1.5 | 0.9 | 1.0 | 0.9 | 1.0 | 1.0 | 1.0 | 4.6 |
| 48362 | FBN2 | FBN2 | fibrillin 2 | 0.9 | 1.0 | 1.5 | 1.4 | 1.0 | 1.0 | 1.3 | 1.3 | 0.9 | 1.0 | 4.6 |
| | | | (congenital | | | | | | | | | | | |
| | | | contractural | | | | | | | | | | | |
| | | | arachnodactyl | | | | | | | | | | | |
| 48839 | FCGR1A | | | 0.8 | 1.1 | 2.2 | 2.1 | 1.2 | 1.2 | 1.4 | 1.3 | 1.1 | 1.0 | 5.5 |
| 48273 | FCGR2A | CD32 | Fc fragment of | 2.0 | 1.9 | 3.8 | 2.5 | 1.0 | 1.0 | [symbol] | 2.3 | [symbol] | 1.3 | 7.0 |
| | | | IgG, low affinity | | | | | | | | | | | |
| | | | Ila, receptor for | | | | | | | | | | | |
| 50715 | FCGR2A | CD32 | Fc fragment of | 1.7 | 1.7 | 3.8 | 3.1 | 1.1 | 1.1 | [symbol] | 2.5 | 1.3 | 1.2 | 7.8 |
| | | | IgG, low affinity | | | | | | | | | | | |
| | | | Ila, receptor for | | | | | | | | | | | |
| 48977 | FCGR2A | CD32 | Fc fragment of | 2.1 | 1.9 | 3.0 | 2.1 | 1.0 | 1.0 | [symbol] | 2.5 | 1.2 | 1.2 | 6.9 |
| | | | IgG, low affinity | | | | | | | | | | | |
| | | | Ila, receptor for | | | | | | | | | | | |
| 49995 | FCGR2A | CD32 | Fc fragment of | 2.4 | 2.0 | 3.6 | 2.7 | 1.1 | 1.0 | [symbol] | 2.5 | 1.2 | 1.2 | 7.4 |
| | | | IgG, low affinity | | | | | | | | | | | |
| | | | Ila, receptor for | | | | | | | | | | | |
| 49326 | FCGR2A | CD32 | Fc fragment of | 2.5 | 2.1 | 3.2 | 2.1 | 1.0 | 1.0 | [symbol] | 1.9 | 1.2 | 1.1 | 6.0 |
| | | | IgG, low affinity | | | | | | | | | | | |
| | | | Ila, receptor for | | | | | | | | | | | |
| 49649 | FGF2 | FGF-2 | fibroblast growth | 3.0 | 2.8 | 1.0 | 1.0 | 0.9 | 1.0 | [symbol] | 2.1 | 0.9 | 1.0 | 5.1 |
| | | | factor 2 (basic) | | | | | | | | | | | |
| 50729 | FGF7 | FGF7 | fibroblast growth | 3.2 | 2.7 | 1.0 | 1.0 | 1.0 | 1.0 | 1.0 | 1.0 | 1.0 | 1.0 | 4.0 |
| | | | factor 7 | | | | | | | | | | | |
| | | | (keratinocyte | | | | | | | | | | | |
| | | | growth fa | | | | | | | | | | | |
| 48946 | FGFR1 | FGF receptor 1 | fibroblast growth | 0.7 | 1.3 | 1.7 | 1.6 | 1.0 | 1.0 | [symbol] | 1.5 | 1.0 | 1.0 | 5.1 |
| | | | factor receptor 1 | | | | | | | | | | | |
| | | | (fms-related t | | | | | | | | | | | |
| 49493 | FLJ10430 | | | 0.9 | 1.0 | 0.7 | 1.4 | 0.9 | 1.1 | 1.0 | 1.0 | 0.9 | 1.0 | 4.4 |
| 47385 | FLJ10718 | | | 3.2 | 2.7 | 0.4 | 1.9 | 0.7 | 1.3 | 0.8 | 1.0 | 1.0 | 1.0 | 5.2 |
| 48328 | FN1 | FN1 | fibronectin 1 | 0.9 | 1.1 | 0.0 | 10.1 | 0.7 | 1.4 | [symbol] | 12.4 | [symbol] | 1.3 | 25.2 |
| 48348 | FN1 | FN1 | fibronectin 1 | 0.8 | 1.1 | 0.0 | 9.6 | 0.6 | 1.4 | [symbol] | 7.6 | 0.8 | 1.2 | 19.9 |
| 47566 | FOS | c-fos | v-fos FBJ murine | 0.8 | 1.2 | 1.4 | 1.3 | 0.9 | 1.0 | 0.8 | 1.2 | 0.7 | 1.1 | 4.7 |
| | | | osteosarcoma | | | | | | | | | | | |
| | | | viral oncogene | | | | | | | | | | | |
| | | | homol | | | | | | | | | | | |
| 49617 | FOXF1 | FOXF1 | forkhead box F1 | 3.3 | 1.7 | 1.4 | 1.1 | 1.0 | 1.0 | [symbol] | 1.8 | 1.3 | 1.2 | 5.1 |
| 49969 | FOXF1 | FOXF1 | forkhead box F1 | 3.4 | 1.7 | 1.4 | 1.1 | 1.0 | 1.0 | [symbol] | 1.7 | 1.3 | 1.1 | 4.9 |
| 46564 | FYB | | | 2.3 | 1.7 | 1.3 | 1.2 | 1.0 | 1.0 | 1.6 | 1.3 | 1.3 | 1.0 | 4.5 |
| 48467 | GAA | | | 0.8 | 1.2 | 1.3 | 1.2 | 1.1 | 1.0 | 1.6 | 1.4 | 1.2 | 1.1 | 4.7 |
| 47169 | GAB1 | Gab1 | GRB2-associated | 3.5 | 3.0 | 1.0 | 1.0 | 1.0 | 1.0 | 1.0 | 1.0 | 1.1 | 1.0 | 4.0 |
| | | | binding protein 1 | | | | | | | | | | | |
| 47386 | GALNAC4S-6ST | BRAG | B cell RAG | 0.7 | 1.0 | 0.6 | 1.5 | 1.0 | 1.0 | 0.6 | 1.4 | 0.8 | 1.1 | 5.0 |
| | | | associated protein | | | | | | | | | | | |
| 49669 | GALNAC4S-6ST | BRAG | B cell RAG | 0.5 | 1.5 | 0.5 | 1.7 | 1.0 | 1.0 | [symbol] | 1.8 | 0.7 | 1.0 | 5.5 |
| | | | associated protein | | | | | | | | | | | |
| 49626 | GARP | | | 3.2 | 2.5 | 1.0 | 1.0 | 1.0 | 1.0 | 0.8 | 1.0 | 1.0 | 1.0 | 4.0 |
| 50142 | GBP1 | IFN induced | guanylate binding | 26.3 | 17.4 | 1.4 | 1.3 | 1.2 | 1.1 | 1.0 | 1.0 | 1.2 | 1.1 | 4.6 |
| | | GBP-1 | protein 1, | | | | | | | | | | | |
| | | | interferon- | | | | | | | | | | | |
| | | | inducible, | | | | | | | | | | | |
| 48066 | GBP2 | IFN induced | guanylate binding | 13.0 | 12.4 | 1.3 | 1.0 | 1.1 | 1.0 | 0.9 | 1.0 | 1.2 | 1.1 | 4.1 |
| | | GBP-2 | protein 2, | | | | | | | | | | | |
| | | | interferon- | | | | | | | | | | | |
| | | | inducible | | | | | | | | | | | |
| 50373 | GBP2 | IFN induced | guanylate binding | 30.2 | 29.9 | 1.0 | 1.0 | 1.0 | 1.0 | 1.0 | 1.0 | 1.2 | 1.0 | 4.0 |
| | | GBP-2 | protein 2, | | | | | | | | | | | |
| | | | interferon- | | | | | | | | | | | |
| | | | inducible | | | | | | | | | | | |
| 49092 | GCH1 | GTP | GTP | 20.6 | 20.1 | 1.0 | 1.0 | 0.9 | 1.0 | 0.9 | 1.0 | [symbol] | 1.3 | 4.3 |
| | | cyclohydrolase I | cyclohydrolase 1 | | | | | | | | | | | |
| | | | (dopa-responsive | | | | | | | | | | | |
| | | | dystonia) | | | | | | | | | | | |
| 46977 | GCH1 | GTP | GTP | 15.5 | 14.6 | 0.5 | 1.4 | 1.0 | 1.0 | 0.8 | 1.0 | [symbol] | 1.3 | 4.7 |
| | | cyclohydrolase I | cyclohydrolase 1 | | | | | | | | | | | |
| | | | (dopa-responsive | | | | | | | | | | | |
| | | | dystonia) | | | | | | | | | | | |
| 49059 | GCH1 | GTP | GTP | 18.7 | 18.2 | 1.0 | 1.0 | 0.8 | 1.0 | 0.9 | 1.0 | 1.3 | 1.1 | 4.1 |
| | | cyclohydrolase I | cyclohydrolase 1 | | | | | | | | | | | |
| | | | (dopa-responsive | | | | | | | | | | | |
| | | | dystonia) | | | | | | | | | | | |
| 49023 | GGA2 | | | 0.5 | 2.1 | 1.0 | 1.0 | 1.0 | 1.0 | 0.8 | 1.1 | 0.9 | 1.0 | 4.1 |
| 48293 | GGT1 | | | 0.9 | 1.0 | 1.6 | 1.3 | 1.1 | 1.0 | [symbol] | 1.5 | 1.2 | 1.1 | 4.9 |
| 48651 | GLA | | | 1.3 | 1.2 | 1.4 | 1.3 | 1.0 | 1.0 | 1.4 | 1.2 | 1.1 | 1.0 | 4.6 |
| 50653 | GM2A | | | 0.9 | 1.0 | 1.7 | 1.6 | 1.1 | 1.0 | [symbol] | 1.6 | 1.2 | 1.1 | 5.3 |
| 49017 | GMPR | | | 2.8 | 2.1 | 1.3 | 1.1 | 1.0 | 1.0 | 1.1 | 1.0 | 1.1 | 1.0 | 4.2 |
| 48722 | GNA13 | G protein, | guanine | 2.5 | 2.1 | 0.9 | 1.0 | 1.0 | 1.0 | 1.1 | 1.0 | 1.1 | 1.0 | 4.1 |
| | | alpha 13 | nucleotide binding | | | | | | | | | | | |
| | | | protein (G | | | | | | | | | | | |
| | | | protein), al | | | | | | | | | | | |
| 50020 | GNAI1 | G protein, | guanine | 0.4 | 2.2 | 0.6 | 1.3 | 1.0 | 1.0 | 1.0 | 1.0 | 1.0 | 1.0 | 4.3 |
| | | alpha i1 | nucleotide binding | | | | | | | | | | | |
| | | | protein (G | | | | | | | | | | | |
| | | | protein), al | | | | | | | | | | | |
| 50433 | GNAS | G protein, | guanine | 0.9 | 1.1 | 1.6 | 1.4 | 1.1 | 1.0 | [symbol] | 1.7 | 1.1 | 1.0 | 5.1 |
| | | alpha s1 | nucleotide binding | | | | | | | | | | | |
| | | | protein (G | | | | | | | | | | | |
| | | | protein), al | | | | | | | | | | | |
| 47648 | GNAS | G protein, | guanine | 0.8 | 1.1 | 1.5 | 1.3 | 1.0 | 1.0 | [symbol] | 1.6 | 1.1 | 1.0 | 4.9 |
| | | alpha s1 | nucleotide binding | | | | | | | | | | | |
| | | | protein (G | | | | | | | | | | | |
| | | | protein), al | | | | | | | | | | | |
| 50746 | GNAS | G protein, | guanine | 0.9 | 1.1 | 1.5 | 1.3 | 1.1 | 1.0 | [symbol] | 1.5 | 1.0 | 1.0 | 4.9 |
| | | alpha s1 | nucleotide binding | | | | | | | | | | | |
| | | | protein (G | | | | | | | | | | | |
| | | | protein), al | | | | | | | | | | | |
| 50743 | GNB1 | G protein, beta 1 | guanine | 0.9 | 1.0 | 0.7 | 1.4 | 1.1 | 1.1 | 0.7 | 1.4 | 1.0 | 1.0 | 4.9 |
| | | | nucleotide binding | | | | | | | | | | | |
| | | | protein (G | | | | | | | | | | | |
| | | | protein), be | | | | | | | | | | | |
| 49769 | GNG10 | G protein, | guanine | 1.1 | 1.0 | 1.3 | 1.1 | 1.1 | 1.0 | 1.4 | 1.3 | 1.1 | 1.0 | 4.4 |
| | | gamma 10 | nucleotide binding | | | | | | | | | | | |
| | | | protein 10 | | | | | | | | | | | |
| 46876 | GNG11 | G protein, | guanine | 1.2 | 1.0 | 0.4 | 2.6 | 0.8 | 1.1 | [symbol] | 2.8 | 0.9 | 1.0 | 7.5 |
| | | gamma 11 | nucleotide binding | | | | | | | | | | | |
| | | | protein 11 | | | | | | | | | | | |
| 49277 | GNG11 | G protein, | guanine | 1.1 | 1.0 | 0.5 | 1.6 | 0.8 | 1.1 | [symbol] | 1.9 | 1.0 | 1.0 | 5.6 |
| | | gamma 11 | nucleotide binding | | | | | | | | | | | |
| | | | protein 11 | | | | | | | | | | | |
| 47511 | GNLY | NKG5 | granulysin | 1.2 | 1.0 | 0.6 | 1.3 | 0.8 | 1.1 | 0.7 | 1.0 | 1.0 | 1.0 | 4.5 |
| 46780 | GPA33 | | | 11.3 | 7.9 | 0.9 | 1.0 | 1.0 | 1.0 | 1.5 | 1.2 | [symbol] | 1.4 | 4.6 |
| 46936 | GRB10 | GRB10 | growth factor | 3.9 | 2.8 | 0.9 | 1.0 | 0.9 | 1.0 | 0.9 | 1.0 | 1.2 | 1.0 | 4.0 |
| | | | receptor-bound | | | | | | | | | | | |
| | | | protein 10 | | | | | | | | | | | |
| 50674 | GRF2 | C3G | guanine | 0.9 | 1.0 | 1.2 | 1.1 | 1.1 | 1.0 | [symbol] | 1.5 | 1.1 | 1.0 | 4.6 |
| | | | nucleotide- | | | | | | | | | | | |
| | | | releasing factor 2 | | | | | | | | | | | |
| | | | (specific fo | | | | | | | | | | | |
| 50545 | GRF2 | C3G | guanine | 0.9 | 1.0 | 1.2 | 1.1 | 1.0 | 1.0 | 1.5 | 1.4 | 1.1 | 1.0 | 4.5 |
| | | | nucleotide- | | | | | | | | | | | |
| | | | releasing factor 2 | | | | | | | | | | | |
| | | | (specific fo | | | | | | | | | | | |
| 49586 | GRIN2C | | | 7.9 | 7.7 | 0.2 | 4.4 | 0.7 | 1.4 | 0.5 | 1.3 | 0.8 | 1.0 | 8.1 |
| 49754 | GRO1 | GRO1 | GRO1 oncogene | 44.5 | 27.1 | 0.4 | 2.2 | 0.7 | 1.3 | 0.8 | 1.0 | 1.0 | 1.0 | 5.5 |
| | | | (melanoma growth | | | | | | | | | | | |
| | | | stimulating activit | | | | | | | | | | | |
| 50462 | GRO1 | GRO1 | GRO1 oncogene | 33.8 | 21.0 | 0.4 | 2.1 | 0.8 | 1.2 | 0.7 | 1.0 | 1.0 | 1.0 | 5.3 |
| | | | (melanoma growth | | | | | | | | | | | |
| | | | stimulating activit | | | | | | | | | | | |
| 47558 | GRO2 | GRO2 | GRO2 oncogene | 44.6 | 15.5 | 1.0 | 1.0 | 0.8 | 1.0 | 0.7 | 1.1 | 0.9 | 1.1 | 4.3 |
| 47632 | GRO2 | GRO2 | GRO2 oncogene | 25.4 | 16.7 | 0.4 | 2.5 | 0.7 | 1.4 | 0.7 | 1.0 | 0.9 | 1.0 | 5.9 |
| 48330 | GRO3 | GRO3 | GRO3 oncogene | 36.2 | 17.9 | 0.4 | 2.2 | 0.7 | 1.2 | 0.8 | 1.0 | 0.9 | 1.1 | 5.5 |
| 49692 | GRO3 | GRO3 | GRO3 oncogene | 33.5 | 17.1 | 0.4 | 2.2 | 0.7 | 1.4 | 0.7 | 1.0 | 1.0 | 1.0 | 5.6 |
| 48455 | GRP58 | | | 0.9 | 1.0 | 1.3 | 1.2 | 1.0 | 1.0 | 1.4 | 1.3 | 1.2 | 1.1 | 4.7 |
| 46895 | GS3686 | | | 6.6 | 6.0 | 1.6 | 1.5 | 1.1 | 1.0 | 0.9 | 1.1 | 1.1 | 1.0 | 4.5 |
| 49688 | GSK3B | GSK3B | glycogen synthase | 1.0 | 1.0 | 1.3 | 1.2 | 1.0 | 1.0 | 1.3 | 1.3 | 1.1 | 1.0 | 4.5 |
| | | | kinase 3 beta | | | | | | | | | | | |
| 47982 | GSTA4 | | | 0.9 | 1.0 | 1.6 | 1.4 | 1.1 | 1.0 | 1.4 | 1.1 | 0.9 | 1.0 | 4.5 |
| 47753 | GTF2B | | | 3.2 | 2.9 | 1.0 | 1.0 | 1.0 | 1.0 | 1.3 | 1.0 | 1.1 | 1.0 | 4.0 |
| 48780 | GZMA | granzyme A | granzyme A | 3.4 | 3.0 | 1.2 | 1.0 | 1.0 | 1.0 | 1.4 | 1.2 | 1.0 | 1.0 | 4.2 |
| | | | (granzyme 1, | | | | | | | | | | | |
| | | | cytotoxic T- | | | | | | | | | | | |
| | | | lymphocyte-ass | | | | | | | | | | | |
| 46882 | HEXA | | | 0.9 | 1.0 | 1.4 | 1.1 | 1.1 | 1.1 | 1.6 | 1.4 | 1.1 | 1.1 | 4.7 |
| 48397 | HIF1A | HIF-1 alpha | hypoxia-inducible | 4.1 | 3.9 | 0.8 | 1.2 | 1.1 | 1.0 | 1.0 | 1.0 | 1.1 | 1.0 | 4.2 |
| | | | factor 1, alpha | | | | | | | | | | | |
| | | | subunit (basic h | | | | | | | | | | | |
| 50022 | HIF1A | HIF-1 alpha | hypoxia-inducible | 3.9 | 3.4 | 0.9 | 1.1 | 1.1 | 1.0 | 1.0 | 1.0 | 1.1 | 1.0 | 4.2 |
| | | | factor 1, alpha | | | | | | | | | | | |
| | | | subunit (basic h | | | | | | | | | | | |
| 50381 | HLA-DQB1 | MHCII, DQ | major | 1.0 | 1.0 | 2.0 | 1.7 | 1.2 | 1.0 | [symbol] | 1.6 | 1.0 | 1.0 | 5.3 |
| | | beta 1 | histocompatibility | | | | | | | | | | | |
| | | | complex, class II, | | | | | | | | | | | |
| | | | DQ bet | | | | | | | | | | | |
| 47179 | HLA-DRB1 | MHCII, DR | major | 0.9 | 1.0 | 2.0 | 1.7 | 1.2 | 1.1 | [symbol] | 1.8 | 1.3 | 1.2 | 5.8 |
| | | beta 1 | histocompatibility | | | | | | | | | | | |
| | | | complex, class II, | | | | | | | | | | | |
| | | | DR bet | | | | | | | | | | | |
| 49305 | HLA-DRB1 | MHCII, DR | major | 0.9 | 1.0 | 2.1 | 1.8 | 1.1 | 1.1 | [symbol] | 1.8 | 1.2 | 1.2 | 6.0 |
| | | beta 1 | histocompatibility | | | | | | | | | | | |
| | | | complex, class II, | | | | | | | | | | | |
| | | | DR bet | | | | | | | | | | | |
| 47887 | HLA-DRB1 | MHCII, DR | major | 1.1 | 1.0 | 1.8 | 1.7 | 1.1 | 1.0 | [symbol] | 1.7 | 1.2 | 1.2 | 5.6 |
| | | beta 1 | histocompatibility | | | | | | | | | | | |
| | | | complex, class II, | | | | | | | | | | | |
| | | | DR bet | | | | | | | | | | | |
| 46926 | HLA-DRB1 | MHCII, DR | major | 1.0 | 1.0 | 2.0 | 1.5 | 1.2 | 1.1 | [symbol] | 1.7 | 1.3 | 1.2 | 5.6 |
| | | beta 1 | histocompatibility | | | | | | | | | | | |
| | | | complex, class II, | | | | | | | | | | | |
| | | | DR bet | | | | | | | | | | | |
| 48589 | HLA-DRB1 | MHCII, DR | major | 1.0 | 1.0 | 2.0 | 1.8 | 1.1 | 1.1 | [symbol] | 1.7 | 1.2 | 1.2 | 5.7 |
| | | beta 1 | histocompatibility | | | | | | | | | | | |
| | | | complex, class II, | | | | | | | | | | | |
| | | | DR bet | | | | | | | | | | | |
| 50385 | HLA-DRB1 | MHCII, DR | major | 0.9 | 1.1 | 1.9 | 1.7 | 1.1 | 1.0 | [symbol] | 1.8 | 1.2 | 1.0 | 5.5 |
| | | beta 1 | histocompatibility | | | | | | | | | | | |
| | | | complex, class II, | | | | | | | | | | | |
| | | | DR bet | | | | | | | | | | | |
| 49293 | HLA-DRB3 | MHCII, DR | major | 0.9 | 1.0 | 2.0 | 1.8 | 1.1 | 1.1 | [symbol] | 1.7 | 1.1 | 1.1 | 5.8 |
| | | beta 3 | histocompatibility | | | | | | | | | | | |
| | | | complex, class II, | | | | | | | | | | | |
| | | | DR bet | | | | | | | | | | | |
| 47855 | HM74 | GPGR | putative | 1.8 | 1.7 | 1.2 | 1.2 | 1.1 | 1.0 | 1.4 | 1.3 | 1.2 | 1.1 | 4.6 |
| | | | chemokine | | | | | | | | | | | |
| | | | receptor; GTP- | | | | | | | | | | | |
| | | | binding protein | | | | | | | | | | | |
| 49217 | HMGIC | | | 0.6 | 1.1 | 0.7 | 1.3 | 0.9 | 1.0 | [symbol] | 1.8 | [symbol] | 1.3 | 5.4 |
| 50361 | HMGIY | HMG-I/Y | high-mobility | 0.7 | 1.3 | 0.5 | 1.7 | 0.9 | 1.1 | [symbol] | 1.5 | 0.8 | 1.1 | 5.4 |
| | | | group (nonhistone | | | | | | | | | | | |
| | | | chromosomal) | | | | | | | | | | | |
| | | | prote | | | | | | | | | | | |
| 50526 | HMGIY | HMG-I/Y | high-mobility | 0.7 | 1.4 | 0.5 | 1.6 | 0.9 | 1.1 | 0.6 | 1.4 | 0.8 | 1.1 | 5.2 |
| | | | group (nonhistone | | | | | | | | | | | |
| | | | chromosomal) | | | | | | | | | | | |
| | | | prote | | | | | | | | | | | |
| 50493 | HMGIY | HMG-I/Y | high-mobility | 0.7 | 1.4 | 0.6 | 1.4 | 0.9 | 1.1 | 0.6 | 1.4 | 0.8 | 1.1 | 5.0 |
| | | | group (nonhistone | | | | | | | | | | | |
| | | | chromosomal) | | | | | | | | | | | |
| | | | prote | | | | | | | | | | | |
| 49347 | HNRPA3 | | | 0.9 | 1.1 | 0.5 | 1.7 | 1.0 | 1.0 | [symbol] | 1.7 | 1.0 | 1.0 | 5.4 |
| 47605 | HRMT1L2 | | | 1.3 | 1.2 | 0.5 | 1.8 | 0.8 | 1.1 | 0.6 | 1.4 | 0.9 | 1.0 | 5.3 |
| 47922 | HS3ST3A1 | HS3ST3A1 | heparan sulfate | 2.3 | 2.1 | 0.6 | 1.3 | 0.6 | 1.2 | 1.1 | 1.0 | 1.0 | 1.0 | 4.5 |
| | | | (glucosamine) 3- | | | | | | | | | | | |
| | | | O-sulfotransferase | | | | | | | | | | | |
| 47185 | HSPA1B | HSPA1B | heat shock 70 kD | 2.0 | 1.7 | 1.6 | 1.5 | 1.1 | 1.0 | [symbol] | 1.5 | 1.3 | 1.2 | 5.1 |
| | | | protein 1B | | | | | | | | | | | |
| 49417 | HSPA1B | HSPA1B | heat shock 70 kD | 2.2 | 1.4 | 1.7 | 1.5 | 1.1 | 1.0 | [symbol] | 1.6 | 1.3 | 1.1 | 5.2 |
| | | | protein 1B | | | | | | | | | | | |
| 48618 | HSPA1B | HSPA1B | heat shock 70 kD | 2.2 | 1.5 | 1.6 | 1.5 | 1.1 | 1.0 | 1.8 | 1.4 | 1.3 | 1.1 | 4.9 |
| | | | protein 1B | | | | | | | | | | | |
| 49064 | HSPA1B | HSPA1B | heat shock 70 kD | 2.4 | 1.6 | 1.6 | 1.4 | 1.1 | 1.0 | 1.8 | 1.4 | 1.2 | 1.0 | 4.7 |
| | | | protein 1B | | | | | | | | | | | |
| 48421 | HSPB2 | HSPB2 | heat shock 27 kD | 4.1 | 3.4 | 1.0 | 1.0 | 1.0 | 1.0 | 0.8 | 1.0 | 1.1 | 1.0 | 4.0 |
| | | | protein 2 | | | | | | | | | | | |
| 48550 | HSPB2 | HSPB2 | heat shock 27 kD | 4.1 | 3.2 | 1.1 | 1.0 | 1.0 | 1.0 | 1.0 | 1.0 | 1.1 | 1.0 | 4.0 |
| | | | protein 2 | | | | | | | | | | | |
| 48693 | HSPBP1 | HSPBP1 | hsp70-interacting | 0.9 | 1.0 | 0.7 | 1.2 | 0.9 | 1.0 | 0.7 | 1.3 | 1.0 | 1.0 | 4.5 |
| | | | protein | | | | | | | | | | | |
| 49681 | HSXIAPAF1 | XIAP assoc | XIAP associated | 9.5 | 9.3 | 2.8 | 2.6 | 1.1 | 1.0 | 1.0 | 1.0 | 0.9 | 1.0 | 5.6 |
| | | factor 1 | factor-1 | | | | | | | | | | | |
| 48605 | HSXIAPAF1 | XIAP assoc | XIAP associated | 13.2 | 8.9 | 2.7 | 2.5 | 1.2 | 1.0 | 1.1 | 1.0 | 1.0 | 1.0 | 5.6 |
| | | factor 1 | factor-1 | | | | | | | | | | | |
| 47611 | HTATIP | | | 1.2 | 1.1 | 0.7 | 1.3 | 1.0 | 1.0 | 0.8 | 1.2 | 1.0 | 1.0 | 4.5 |
| 48199 | HXB | HXB | hexabrachion | 3.0 | 2.3 | 1.2 | 1.0 | 1.0 | 1.0 | 1.2 | 1.0 | 0.9 | 1.0 | 4.0 |
| | | | (tenascin C, | | | | | | | | | | | |
| | | | cytotactin) | | | | | | | | | | | |
| 49140 | ICAM1 | ICAM1 | intercellular | 6.0 | 4.2 | 0.7 | 1.2 | 0.9 | 1.0 | 1.0 | 1.0 | 1.1 | 1.0 | 4.2 |
| | | | adhesion | | | | | | | | | | | |
| | | | molecule 1 | | | | | | | | | | | |
| | | | (CD54), human rh | | | | | | | | | | | |
| 49771 | ICAM1 | ICAM1 | intercellular | 6.3 | 4.5 | 0.7 | 1.3 | 0.9 | 1.1 | 0.9 | 1.0 | 1.1 | 1.0 | 4.4 |
| | | | adhesion | | | | | | | | | | | |
| | | | molecule 1 | | | | | | | | | | | |
| | | | (CD54), human rh | | | | | | | | | | | |
| 49804 | ICAM1 | ICAM1 | intercellular | 6.0 | 4.4 | 0.7 | 1.3 | 0.9 | 1.1 | 1.0 | 1.0 | 1.1 | 1.0 | 4.4 |
| | | | adhesion | | | | | | | | | | | |
| | | | molecule 1 | | | | | | | | | | | |
| | | | (CD54), human rh | | | | | | | | | | | |
| 50084 | IDI1 | | | 0.8 | 1.2 | 1.1 | 1.0 | 0.8 | 1.2 | 0.9 | 1.1 | [symbol] | 1.3 | 4.6 |
| 47803 | IFI16 | | | 4.2 | 3.3 | 1.1 | 1.1 | 1.0 | 1.0 | 1.1 | 1.0 | 1.1 | 1.0 | 4.1 |
| 48436 | IFI35 | IFN-induced | interferon-induced | 5.2 | 4.5 | 1.7 | 1.5 | 1.1 | 1.0 | 0.9 | 1.0 | 1.1 | 1.0 | 4.5 |
| | | protein 35 | protein 35 | | | | | | | | | | | |
| 46595 | IFI35 | IFN-induced | interferon-induced | 6.3 | 4.8 | 1.6 | 1.2 | 1.2 | 1.1 | 1.0 | 1.0 | 1.1 | 1.0 | 4.3 |
| | | protein 35 | protein 35 | | | | | | | | | | | |
| 49870 | IFIT4 | | | 10.9 | 6.3 | 1.1 | 1.0 | 0.7 | 1.2 | [symbol] | 1.7 | 1.2 | 1.2 | 5.2 |
| 48115 | IFITM3 | | | 4.3 | 3.1 | 1.7 | 1.6 | 1.1 | 1.1 | 1.2 | 1.1 | 1.1 | 1.1 | 4.9 |
| 50311 | IFNGR1 | IFN gamma | interferon gamma | 0.7 | 1.2 | 2.3 | 1.8 | 1.2 | 1.0 | 1.3 | 1.1 | 0.9 | 1.0 | 5.0 |
| | | receptor 1 | receptor 1 | | | | | | | | | | | |
| 47903 | IFNGR1 | IFN gamma | interferon gamma | 0.7 | 1.0 | 2.0 | 1.5 | 1.1 | 1.0 | 1.4 | 1.3 | 1.0 | 1.0 | 4.9 |
| | | receptor 1 | receptor 1 | | | | | | | | | | | |
| 47225 | IFNGR2 | IFN gamma | interferon gamma | 1.3 | 1.0 | 0.7 | 1.3 | 0.9 | 1.0 | 1.0 | 1.0 | 1.0 | 1.0 | 4.3 |
| | | receptor 2 | receptor 2 | | | | | | | | | | | |
| | | | (interferon gamma | | | | | | | | | | | |
| | | | tran | | | | | | | | | | | |
| 47582 | IFNGR2 | IFN gamma | interferon gamma | 1.3 | 1.0 | 0.7 | 1.3 | 0.9 | 1.0 | 1.1 | 1.0 | 1.0 | 1.0 | 4.3 |
| | | receptor 2 | receptor 2 | | | | | | | | | | | |
| | | | (interferon gamma | | | | | | | | | | | |
| | | | tran | | | | | | | | | | | |
| 50403 | IGFBP3 | | | 2.8 | 2.2 | 0.2 | 4.2 | 0.9 | 1.0 | [symbol] | 3.8 | 0.7 | 1.1 | 10.1 |
| 50342 | IKKE | IKK-related K | IKK-related kinase | 1.1 | 1.0 | 1.5 | 1.3 | 1.0 | 1.0 | 1.3 | 1.0 | 1.0 | 1.0 | 4.3 |
| | | epsilon, IKK-I | epsilon; inducible | | | | | | | | | | | |
| | | | IkappaB kina | | | | | | | | | | | |
| 49758 | IL10RA | IL-10R alpha | interleukin 10 | 9.5 | 7.9 | 2.0 | 1.8 | 1.2 | 1.0 | [symbol] | 2.1 | 1.4 | 1.2 | 6.1 |
| | | | receptor, alpha | | | | | | | | | | | |
| 47040 | IL10RA | IL-10R alpha | interleukin 10 | 11.8 | 10.6 | 2.1 | 1.7 | 1.1 | 1.0 | [symbol] | 1.8 | 1.5 | 1.2 | 5.8 |
| | | | receptor, alpha | | | | | | | | | | | |
| 48581 | IL13RA1 | IL-13Ralpha′ | interleukin 13 | 1.9 | 1.6 | 1.7 | 1.5 | 1.0 | 1.0 | 1.1 | 1.1 | 0.9 | 1.0 | 4.6 |
| | | | receptor, alpha 1 | | | | | | | | | | | |
| 50013 | IL13RA1 | IL-13Ralpha′ | interleukin 13 | 1.7 | 1.5 | 1.5 | 1.4 | 1.1 | 1.0 | 1.1 | 1.0 | 1.0 | 1.0 | 4.5 |
| | | | receptor, alpha 1 | | | | | | | | | | | |
| 48909 | IL15RA | IL-15R alpha | interleukin 15 | 11.9 | 9.5 | 0.9 | 1.0 | 1.0 | 1.0 | 1.0 | 1.0 | 1.3 | 1.2 | 4.2 |
| | | | receptor, alpha | | | | | | | | | | | |
| 50667 | IL15RA | IL-15R alpha | interleukin 15 | 13.9 | 13.4 | 1.0 | 1.0 | 1.0 | 1.0 | 1.1 | 1.1 | 1.3 | 1.2 | 4.3 |
| | | | receptor, alpha | | | | | | | | | | | |
| 47656 | IL1B | IL-1, beta | interleukin 1, beta | 2.5 | 2.4 | 0.3 | 3.5 | 0.7 | 1.1 | 1.3 | 1.1 | 1.2 | 1.1 | 6.8 |
| 46970 | IL1B | IL-1, beta | interleukin 1, beta | 3.3 | 2.3 | 0.2 | 3.9 | 0.7 | 1.2 | 1.2 | 1.2 | 1.2 | 1.1 | 7.3 |
| 48637 | IL1R1 | IL-1RI | interleukin 1 | 2.8 | 2.5 | 0.7 | 1.4 | 0.9 | 1.0 | 1.1 | 1.1 | 1.2 | 1.1 | 4.6 |
| | | | receptor, type I | | | | | | | | | | | |
| 47659 | IL1RAP | IL-1RAP | interleukin 1 | 5.6 | 5.2 | 1.0 | 1.0 | 1.0 | 1.0 | 0.9 | 1.0 | 0.9 | 1.0 | 4.0 |
| | | | receptor | | | | | | | | | | | |
| | | | accessory protein | | | | | | | | | | | |
| 47692 | IL1RAP | IL-1RAP | interleukin 1 | 5.1 | 4.6 | 1.0 | 1.0 | 1.0 | 1.0 | 0.9 | 1.0 | 1.1 | 1.0 | 4.0 |
| | | | receptor | | | | | | | | | | | |
| | | | accessory protein | | | | | | | | | | | |
| 48324 | IL3RA | IL-3R alpha | interleukin 3 | 3.4 | 3.1 | 1.4 | 1.2 | 1.0 | 1.0 | 0.8 | 1.0 | 0.8 | 1.1 | 4.2 |
| | | | receptor, alpha | | | | | | | | | | | |
| | | | (low affinity) | | | | | | | | | | | |
| 49135 | IL6 | interleukin-6, | interleukin 6 | 13.6 | 11.3 | 1.3 | 1.2 | 1.0 | 1.0 | 1.3 | 1.0 | [symbol] | 1.4 | 4.6 |
| | | IFNbeta2 | (interferon, beta 2) | | | | | | | | | | | |
| 49264 | IL6 | interleukin-6, | interleukin 6 | 13.5 | 9.7 | 1.4 | 1.2 | 1.0 | 1.0 | 1.3 | 1.0 | [symbol] | 1.4 | 4.6 |
| | | IFNbeta2 | (interferon, beta 2) | | | | | | | | | | | |
| 49289 | IL6R | IL-6R | interleukin 6 | 0.9 | 1.0 | 1.4 | 1.3 | 1.0 | 1.0 | 1.5 | 1.3 | 1.0 | 1.0 | 4.6 |
| | | | receptor | | | | | | | | | | | |
| 47844 | IL7R | IL-7R | interleukin 7 | 20.6 | 16.8 | 0.9 | 1.0 | 1.0 | 1.0 | 0.7 | 1.2 | 1.4 | 1.2 | 4.5 |
| | | | receptor | | | | | | | | | | | |
| 47715 | IL7R | IL-7R | interleukin 7 | 19.9 | 19.3 | 1.0 | 1.0 | 1.0 | 1.0 | 0.8 | 1.3 | 1.4 | 1.2 | 4.5 |
| | | | receptor | | | | | | | | | | | |
| 46856 | IL8 | interleukin-8 | interleukin 8 | 7.2 | 6.7 | 0.2 | 3.8 | 0.6 | 1.4 | 1.2 | 1.2 | 1.2 | 1.0 | 7.4 |
| 50455 | IMPDH1 | | | 1.5 | 1.3 | 1.3 | 1.1 | 1.1 | 1.0 | 1.4 | 1.3 | 1.0 | 1.0 | 4.5 |
| 46724 | INDO | | | 47.1 | 35.0 | 1.0 | 1.0 | 1.0 | 1.0 | 0.9 | 1.0 | 1.4 | 1.2 | 4.3 |
| 49873 | INSIG1 | | | 1.5 | 1.4 | 0.6 | 1.4 | 0.7 | 1.4 | 1.0 | 1.0 | [symbol] | 1.3 | 5.1 |
| 49763 | IQGAP2 | IQGAP2 | IQ motif | 2.2 | 1.8 | 1.2 | 1.0 | 1.1 | 1.0 | 0.7 | 1.4 | 1.0 | 1.0 | 4.4 |
| | | | containing | | | | | | | | | | | |
| | | | GTPase activating | | | | | | | | | | | |
| | | | protein 2 | | | | | | | | | | | |
| 46642 | IRAK2 | IRAK-2 | interleukin-1 | 5.7 | 5.2 | 0.7 | 1.0 | 1.1 | 1.0 | 1.1 | 1.0 | 1.0 | 1.0 | 4.0 |
| | | | receptor- | | | | | | | | | | | |
| | | | associated kinase 2 | | | | | | | | | | | |
| 47186 | IRAK2 | IRAK-2 | interleukin-1 | 5.3 | 4.4 | 0.8 | 1.0 | 1.2 | 1.0 | 1.1 | 1.0 | 1.0 | 1.0 | 4.0 |
| | | | receptor- | | | | | | | | | | | |
| | | | associated kinase 2 | | | | | | | | | | | |
| 49335 | IRF7 | IRF-7A | interferon | 6.9 | 4.5 | 1.4 | 1.2 | 1.0 | 1.0 | 0.9 | 1.1 | 1.0 | 1.0 | 4.3 |
| | | | regulatory factor 7 | | | | | | | | | | | |
| 47149 | IRF7 | IRF-7A | interferon | 4.2 | 3.9 | 1.2 | 1.1 | 1.0 | 1.0 | 1.0 | 1.0 | 1.1 | 1.0 | 4.1 |
| | | | regulatory factor 7 | | | | | | | | | | | |
| 48371 | IRS1 | IRS1 | insulin receptor | 1.0 | 1.0 | 0.5 | 1.3 | 1.0 | 1.0 | 1.0 | 1.0 | 1.0 | 1.0 | 4.3 |
| | | | substrate 1 | | | | | | | | | | | |
| 50580 | ISG15 | ISG15, IFN | interferon- | 17.1 | 15.2 | 1.5 | 1.3 | 1.0 | 1.0 | 1.2 | 1.1 | 1.2 | 1.1 | 4.5 |
| | | stimulated | stimulated protein, | | | | | | | | | | | |
| | | protein | 15 kDa | | | | | | | | | | | |
| 46618 | ISG15 | ISG15, IFN | interferon- | 32.0 | 24.1 | 1.9 | 1.7 | 1.2 | 1.1 | 1.2 | 1.0 | 1.2 | 1.1 | 4.8 |
| | | stimulated | stimulated protein, | | | | | | | | | | | |
| | | protein | 15 kDa | | | | | | | | | | | |
| 50089 | ISG15 | ISG15, IFN | interferon- | 16.8 | 13.5 | 1.7 | 1.5 | 1.1 | 1.0 | 1.3 | 1.2 | 1.1 | 1.0 | 4.7 |
| | | stimulated | stimulated protein, | | | | | | | | | | | |
| | | protein | 15 kDa | | | | | | | | | | | |
| 49054 | ITGA2 | integrin, alpha | integrin, alpha 2 | 0.9 | 1.0 | 0.6 | 1.4 | 0.9 | 1.0 | [symbol] | 1.6 | 1.0 | 1.0 | 5.0 |
| | | 2, CD49B, | (CD49B, alpha 2 | | | | | | | | | | | |
| | | VLA-2 | subunit of VLA-2 | | | | | | | | | | | |
| 48617 | ITGA4 | VLA4a, | integrin, alpha 4 | 0.7 | 1.2 | 1.7 | 1.6 | 1.1 | 1.1 | 1.3 | 1.2 | 1.1 | 1.0 | 4.8 |
| | | integrin, alpha 5 | (antigen CD49D, | | | | | | | | | | | |
| | | | alpha 4 subunit | | | | | | | | | | | |
| 48426 | ITGA4 | VLA4a, | integrin, alpha 4 | 0.5 | 1.7 | 1.5 | 1.4 | 1.2 | 1.0 | 1.4 | 1.3 | 1.1 | 1.0 | 4.7 |
| | | integrin, alpha 5 | (antigen CD49D, | | | | | | | | | | | |
| | | | alpha 4 subunit | | | | | | | | | | | |
| 47138 | ITGAM | Mac-1, | integrin, alpha M | 0.7 | 1.2 | 1.4 | 1.3 | 1.0 | 1.0 | 1.7 | 1.4 | 1.1 | 1.0 | 4.8 |
| | | CD11b, | (complement | | | | | | | | | | | |
| | | integrin alpha M | component | | | | | | | | | | | |
| | | | receptor 3 | | | | | | | | | | | |
| 47009 | ITGAM | Mac-1, | integrin, alpha M | 0.7 | 1.1 | 1.4 | 1.3 | 1.0 | 1.0 | 1.6 | 1.4 | 1.1 | 1.0 | 4.7 |
| | | CD11b, | (complement | | | | | | | | | | | |
| | | integrin alpha M | component | | | | | | | | | | | |
| | | | receptor 3 | | | | | | | | | | | |
| 48628 | ITGB2 | CD18 | integrin, beta 2 | 0.9 | 1.0 | 1.2 | 1.1 | 1.0 | 1.0 | 1.5 | 1.4 | 1.1 | 1.0 | 4.5 |
| | | | (antigen CD18 | | | | | | | | | | | |
| | | | (p95), lymphocyte f | | | | | | | | | | | |
| 50343 | JAK2 | JAK2 | Janus kinase 2 (a | 4.6 | 4.2 | 1.4 | 1.2 | 1.0 | 1.0 | [symbol] | 1.5 | 1.3 | 1.2 | 5.0 |
| | | | protein tyrosine | | | | | | | | | | | |
| | | | kinase) | | | | | | | | | | | |
| 48929 | JAK2 | JAK2 | Janus kinase 2 (a | 4.5 | 4.3 | 1.0 | 1.0 | 1.0 | 1.0 | 1.4 | 1.4 | 1.3 | 1.2 | 4.6 |
| | | | protein tyrosine | | | | | | | | | | | |
| | | | kinase) | | | | | | | | | | | |
| 46749 | JM1 | | | 2.2 | 2.1 | 0.8 | 1.1 | 1.0 | 1.0 | 0.9 | 1.0 | 1.1 | 1.0 | 4.1 |
| 49322 | JUNB | JunB | jun B proto- | 2.4 | 2.1 | 0.9 | 1.0 | 0.9 | 1.1 | 1.1 | 1.1 | 1.0 | 1.0 | 4.2 |
| | | | oncogene | | | | | | | | | | | |
| 49319 | JUNB | JunB | Jun B proto- | 2.3 | 2.0 | 0.9 | 1.0 | 0.9 | 1.1 | 1.2 | 1.1 | 1.0 | 1.0 | 4.1 |
| | | | oncogene | | | | | | | | | | | |
| 47770 | KIAA0062 | | | 3.8 | 3.1 | 0.8 | 1.0 | 0.8 | 1.1 | 0.9 | 1.0 | 1.0 | 1.0 | 4.1 |
| 50052 | KIAA0171 | | | 1.1 | 1.0 | 1.1 | 1.0 | 1.0 | 1.0 | 1.4 | 1.4 | 0.9 | 1.0 | 4.4 |
| 49552 | KIAA0173 | | | 0.9 | 1.0 | 1.3 | 1.2 | 1.0 | 1.0 | 1.4 | 1.4 | 1.0 | 1.0 | 4.6 |
| 48643 | KIAA0184 | | | 1.0 | 1.0 | 0.8 | 1.2 | 1.1 | 1.0 | 0.7 | 1.3 | 1.1 | 1.0 | 4.6 |
| 50764 | KIAA0212 | | | 1.7 | 1.4 | 1.3 | 1.2 | 1.1 | 1.1 | [symbol] | 1.5 | 1.0 | 1.0 | 4.8 |
| 50304 | KIAA0226 | | | 2.2 | 2.1 | 1.2 | 1.1 | 1.1 | 1.0 | 1.3 | 1.1 | 1.1 | 1.0 | 4.2 |
| 48896 | KIAA0227 | | | 0.1 | 7.3 | 0.8 | 1.1 | 1.0 | 1.0 | 0.9 | 1.0 | 0.9 | 1.0 | 4.1 |
| 48668 | KIAA0247 | | | 2.6 | 2.4 | 1.2 | 1.1 | 1.0 | 1.0 | 1.0 | 1.0 | 1.0 | 1.0 | 4.1 |
| 48294 | KIAA0286 | | | 3.5 | 2.8 | 1.0 | 1.0 | 1.0 | 1.0 | 1.0 | 1.0 | 1.2 | 1.1 | 4.1 |
| 50582 | KIAA0534 | | | 2.4 | 2.0 | 1.0 | 1.0 | 1.1 | 1.0 | 1.0 | 1.0 | 1.0 | 1.0 | 4.0 |
| 49904 | KIAA1557 | | | 2.3 | 1.7 | 2.3 | 2.1 | 1.1 | 1.0 | 1.2 | 1.1 | 0.9 | 1.0 | 5.2 |
| 48479 | KIF3C | | | 0.5 | 1.4 | 1.6 | 1.4 | 1.0 | 1.0 | 1.1 | 1.0 | 0.9 | 1.0 | 4.4 |
| 49225 | KMO | | | 2.8 | 2.3 | 1.1 | 1.0 | 1.1 | 1.1 | 1.0 | 1.0 | 1.2 | 1.1 | 4.2 |
| 48990 | KRAS2 | K-Ras | v-Ki-ras2 Kirsten | 1.0 | 1.0 | 0.9 | 1.1 | 1.0 | 1.0 | 0.7 | 1.3 | 1.1 | 1.0 | 4.5 |
| | | | rat sarcoma 2 viral | | | | | | | | | | | |
| | | | oncogene hom | | | | | | | | | | | |
| 47062 | LAIR1 | | | 0.4 | 2.1 | 1.1 | 1.0 | 1.1 | 1.0 | 0.9 | 1.1 | 1.1 | 1.0 | 4.1 |
| 50042 | LAMB3 | LAMB3 | laminin, beta 3 | 4.7 | 3.6 | 1.7 | 1.5 | 1.1 | 1.0 | 0.6 | 1.3 | 0.8 | 1.1 | 4.9 |
| | | | (nicein (125 kD), | | | | | | | | | | | |
| | | | kalinin (140 kD), | | | | | | | | | | | |
| 48692 | LAMC1 | LAMC1 | laminin, gamma 1 | 0.8 | 1.3 | 1.3 | 1.2 | 0.9 | 1.0 | [symbol] | 1.5 | 0.9 | 1.0 | 4.7 |
| | | | (formerly LAMB2) | | | | | | | | | | | |
| 49882 | LAMP2 | | | 0.9 | 1.0 | 1.5 | 1.3 | 1.1 | 1.0 | 1.5 | 1.4 | 1.2 | 1.1 | 4.9 |
| 47033 | LCP2 | SLP-76 | lymphocyte | 3.8 | 3.3 | 0.7 | 1.2 | 0.9 | 1.0 | 1.0 | 1.0 | 1.2 | 1.1 | 4.3 |
| | | | cytosolic protein 2 | | | | | | | | | | | |
| | | | (SH2 domain- | | | | | | | | | | | |
| | | | contain | | | | | | | | | | | |
| 47162 | LCP2 | SLP-76 | lymphocyte | 3.7 | 3.2 | 0.7 | 1.2 | 0.9 | 1.0 | 1.1 | 1.0 | 1.2 | 1.1 | 4.3 |
| | | | cytosolic protein 2 | | | | | | | | | | | |
| | | | (SH2 domain- | | | | | | | | | | | |
| | | | contain | | | | | | | | | | | |
| 49163 | LGALS3 | | | 1.4 | 1.3 | 1.4 | 1.2 | 1.2 | 1.1 | 1.5 | 1.3 | 1.1 | 1.0 | 4.6 |
| 49016 | LIPA | | | 1.1 | 1.0 | 1.4 | 1.3 | 1.1 | 1.0 | [symbol] | 1.6 | 1.2 | 1.1 | 5.0 |
| 50303 | LMO2 | | | 0.9 | 1.0 | 1.9 | 1.4 | 1.2 | 1.1 | 1.4 | 1.3 | 1.0 | 1.0 | 4.8 |
| 50349 | LOC51295 | ECSIT | ECSIT | 0.7 | 1.3 | 0.6 | 1.5 | 1.0 | 1.0 | [symbol] | 1.5 | 0.9 | 1.0 | 5.0 |
| 50618 | LOC51339 | | | 0.4 | 1.9 | 0.7 | 1.0 | 0.7 | 1.3 | 1.0 | 1.0 | 1.0 | 1.0 | 4.3 |
| 49577 | LOC54543 | | | 1.1 | 1.0 | 1.2 | 1.1 | 1.0 | 1.0 | 1.4 | 1.3 | 1.0 | 1.0 | 4.4 |
| 47114 | LPP | | | 1.0 | 1.0 | 1.6 | 1.5 | 1.2 | 1.1 | 1.1 | 1.0 | 1.2 | 1.2 | 4.9 |
| 47585 | LRPAP1 | | | 0.9 | 1.0 | 1.5 | 1.2 | 1.0 | 1.0 | 1.4 | 1.3 | 1.1 | 1.0 | 4.5 |
| 49560 | LY117 | | | 1.0 | 1.0 | 1.5 | 1.4 | 1.0 | 1.0 | 1.7 | 1.4 | 1.0 | 1.0 | 4.9 |
| 46676 | LYN | Lyn | v-yes-1 | 5.7 | 4.6 | 1.0 | 1.0 | 1.0 | 1.0 | 1.4 | 1.4 | 1.2 | 1.2 | 4.5 |
| | | | Yamaguchi | | | | | | | | | | | |
| | | | sarcoma viral | | | | | | | | | | | |
| | | | related oncogene h | | | | | | | | | | | |
| 46605 | LYN | Lyn | v-yes-1 | 5.0 | 3.9 | 0.9 | 1.0 | 1.0 | 1.0 | 1.4 | 1.4 | 1.2 | 1.0 | 4.4 |
| | | | Yamaguchi | | | | | | | | | | | |
| | | | sarcoma viral | | | | | | | | | | | |
| | | | related oncogene h | | | | | | | | | | | |
| 46776 | MAD2L1 | | | 0.7 | 1.2 | 0.4 | 1.6 | 1.0 | 1.0 | 0.6 | 1.1 | 1.0 | 1.0 | 4.6 |
| 47705 | MAD4 | MAD4 | Mad4 homolog | 0.7 | 1.3 | 1.6 | 1.4 | 0.9 | 1.0 | 1.6 | 1.4 | 1.0 | 1.0 | 4.8 |
| 47267 | MAD4 | MAD4 | Mad4 homolog | 0.4 | 2.1 | 0.7 | 1.1 | 1.0 | 1.0 | 0.7 | 1.0 | 0.9 | 1.0 | 4.1 |
| 48028 | MAP2K3 | MKK3 | mitogen-activated | 2.1 | 2.0 | 0.9 | 1.0 | 0.9 | 1.0 | 1.1 | 1.0 | 1.1 | 1.0 | 4.0 |
| | | | protein kinase | | | | | | | | | | | |
| | | | kinase 3 | | | | | | | | | | | |
| 48278 | MAP2K3 | MKK3 | mitogen-activated | 2.2 | 2.1 | 0.9 | 1.0 | 0.9 | 1.0 | 1.1 | 1.0 | 0.9 | 1.0 | 4.0 |
| | | | protein kinase | | | | | | | | | | | |
| | | | kinase 3 | | | | | | | | | | | |
| 49650 | MAP3K10 | MLK2 | mitogen-activated | 0.9 | 1.0 | 1.4 | 1.3 | 1.0 | 1.0 | 1.4 | 1.3 | 1.0 | 1.0 | 4.6 |
| | | | protein kinase | | | | | | | | | | | |
| | | | kinase kinase 10 | | | | | | | | | | | |
| 50694 | MAP3K4 | MEKK4 | mitogen-activated | 1.0 | 1.0 | 0.8 | 1.1 | 0.9 | 1.0 | [symbol] | 1.8 | 0.8 | 1.1 | 4.9 |
| | | | protein kinase | | | | | | | | | | | |
| | | | kinase kinase 4 | | | | | | | | | | | |
| 50150 | MAP3K4 | MEKK4 | mitogen-activated | 0.9 | 1.0 | 0.7 | 1.2 | 0.9 | 1.0 | [symbol] | 2.1 | 0.8 | 1.0 | 5.4 |
| | | | protein kinase | | | | | | | | | | | |
| | | | kinase kinase 4 | | | | | | | | | | | |
| 50043 | MAP3K5 | MKK5, ASK1 | mitogen-activated | 4.5 | 3.4 | 1.3 | 1.2 | 1.0 | 1.0 | 0.9 | 1.0 | 1.0 | 1.0 | 4.2 |
| | | | protein kinase | | | | | | | | | | | |
| | | | kinase kinase 5 | | | | | | | | | | | |
| 50045 | MAP3K5 | MKK5, ASK1 | mitogen-activated | 4.1 | 3.4 | 1.2 | 1.0 | 1.0 | 1.0 | 0.9 | 1.0 | 1.0 | 1.0 | 4.0 |
| | | | protein kinase | | | | | | | | | | | |
| | | | kinase kinase 5 | | | | | | | | | | | |
| 47145 | MAP3K7 | TAK1 | mitogen-activated | 1.1 | 1.0 | 1.0 | 1.0 | 1.0 | 1.0 | 1.3 | 1.3 | 1.1 | 1.0 | 4.3 |
| | | | protein kinase | | | | | | | | | | | |
| | | | kinase kinase 7 | | | | | | | | | | | |
| 47969 | MAP3K8 | c-cot | mitogen-activated | 4.9 | 4.8 | 0.9 | 1.0 | 0.8 | 1.0 | [symbol] | 1.8 | 1.4 | 1.1 | 5.0 |
| | | | protein kinase | | | | | | | | | | | |
| | | | kinase kinase 8 | | | | | | | | | | | |
| 48424 | MAP4K4 | GCK-like, | mitogen-activated | 1.5 | 1.4 | 1.3 | 1.2 | 1.0 | 1.0 | 1.4 | 1.3 | 1.2 | 1.2 | 4.6 |
| | | HPK, HGK, | protein kinase | | | | | | | | | | | |
| | | NIK | kinase kinase kin | | | | | | | | | | | |
| 49437 | MAPK13 | SAPK4, p38, | mitogen-activated | 0.6 | 1.6 | 0.7 | 1.3 | 1.0 | 1.0 | 0.7 | 1.4 | 0.8 | 1.1 | 4.8 |
| | | delta | protein kinase 13 | | | | | | | | | | | |
| 50569 | MBNL | | | 2.2 | 1.8 | 1.4 | 1.3 | 1.0 | 1.0 | 1.6 | 1.4 | 1.3 | 1.1 | 4.8 |
| 47123 | MCM2 | | | 0.6 | 1.7 | 0.5 | 1.3 | 1.0 | 1.0 | 0.7 | 1.2 | 1.1 | 1.0 | 4.5 |
| 48470 | MCM5 | | | 0.7 | 1.5 | 0.6 | 1.3 | 0.9 | 1.1 | [symbol] | 1.5 | 1.0 | 1.0 | 4.9 |
| 47137 | MD-2 | MD-2 | MD-2 protein | 1.0 | 1.0 | 2.1 | 1.7 | 1.1 | 1.0 | 1.6 | 1.4 | 1.2 | 1.2 | 5.2 |
| 46785 | MD-2 | MD-2 | MD-2 protein | 1.0 | 1.0 | 1.9 | 1.3 | 1.1 | 1.1 | 1.6 | 1.4 | 1.1 | 1.1 | 4.8 |
| 46691 | MEF2C | MEF2C | MADS box | 0.8 | 1.0 | 0.9 | 1.1 | 1.0 | 1.0 | 0.7 | 1.4 | 1.0 | 1.0 | 4.5 |
| | | | transcription | | | | | | | | | | | |
| | | | enhancer factor 2, | | | | | | | | | | | |
| | | | polypept | | | | | | | | | | | |
| 50742 | MGEA5 | MGEA5 | meningioma | 0.7 | 1.3 | 1.4 | 1.2 | 1.1 | 1.0 | 1.6 | 1.4 | 0.9 | 1.0 | 4.6 |
| | | | expressed antigen | | | | | | | | | | | |
| | | | 5 (hyaluronidase) | | | | | | | | | | | |
| 48411 | MGEA5 | MGEA5 | meningioma | 0.8 | 1.1 | 1.4 | 1.3 | 1.0 | 1.0 | 1.2 | 1.1 | 1.0 | 1.0 | 4.4 |
| | | | expressed antigen | | | | | | | | | | | |
| | | | 5 (hyaluronidase) | | | | | | | | | | | |
| 47799 | MGLL | | | 1.8 | 1.7 | 1.0 | 1.0 | 1.0 | 1.0 | [symbol] | 1.5 | 1.1 | 1.0 | 4.5 |
| 50404 | MIC2 | | | 1.2 | 1.1 | 1.5 | 1.3 | 1.2 | 1.1 | 1.5 | 1.4 | 1.2 | 1.0 | 4.7 |
| 48570 | MIG | monokine | monokine induced | 15.7 | 10.8 | 0.6 | 1.3 | 0.9 | 1.0 | 1.0 | 1.0 | 1.4 | 1.4 | 4.7 |
| | | induced by | by gamma | | | | | | | | | | | |
| | | gamma | interferon | | | | | | | | | | | |
| | | interferon | | | | | | | | | | | | |
| 48441 | MIG | monokine | monokine induced | 13.7 | 7.6 | 0.6 | 1.2 | 0.8 | 1.1 | 0.9 | 1.0 | 1.4 | 1.3 | 4.6 |
| | | induced by | by gamma | | | | | | | | | | | |
| | | gamma | interferon | | | | | | | | | | | |
| | | interferon | | | | | | | | | | | | |
| 48465 | MIG2 | | | 1.0 | 1.0 | 0.5 | 1.5 | 1.0 | 1.0 | 0.6 | 1.2 | 1.0 | 1.0 | 4.6 |
| 49224 | MITF | | | 0.4 | 1.9 | 1.4 | 1.3 | 1.1 | 1.0 | 1.1 | 1.0 | 1.2 | 1.0 | 4.3 |
| 48966 | MMP10 | MMP10 | matrix | 1.7 | 1.6 | 0.5 | 1.8 | 0.6 | 1.6 | [symbol] | 2.3 | [symbol] | 1.5 | 7.2 |
| | | | metalloproteinase | | | | | | | | | | | |
| | | | 10 (stromelysin 2) | | | | | | | | | | | |
| 47380 | MMP12 | MMP12 | matrix | 2.8 | 1.4 | 0.7 | 1.0 | 0.8 | 1.0 | 0.6 | 1.4 | 1.1 | 1.0 | 4.4 |
| | | | metalloproteinase | | | | | | | | | | | |
| | | | 12 (macrophage | | | | | | | | | | | |
| | | | elastase) | | | | | | | | | | | |
| 48038 | MMP13 | MMP13 | matrix | 0.9 | 1.0 | 1.6 | 1.4 | 1.1 | 1.0 | [symbol] | 1.7 | 1.1 | 1.0 | 5.2 |
| | | | metalloproteinase | | | | | | | | | | | |
| | | | 13 (collagenase 3) | | | | | | | | | | | |
| 48582 | MMP13 | MMP13 | matrix | 0.9 | 1.1 | 1.6 | 1.5 | 1.1 | 1.1 | [symbol] | 1.7 | 1.2 | 1.0 | 5.3 |
| | | | metalloproteinase | | | | | | | | | | | |
| | | | 13 (collagenase 3) | | | | | | | | | | | |
| 50739 | MMP14 | MT1-MMP | matrix | 0.9 | 1.1 | 1.2 | 1.1 | 1.1 | 1.0 | 1.4 | 1.3 | 1.1 | 1.0 | 4.4 |
| | | | metalloproteinase | | | | | | | | | | | |
| | | | 14 (membrane- | | | | | | | | | | | |
| | | | inserted) | | | | | | | | | | | |
| 48949 | MMP2 | MMP2, | matrix | 0.7 | 1.3 | 1.6 | 1.3 | 1.0 | 1.0 | [symbol] | 1.6 | 1.1 | 1.0 | 4.9 |
| | | gelatinase A | metalloproteinase | | | | | | | | | | | |
| | | | 2 (gelatinase A, | | | | | | | | | | | |
| | | | 72 kD gel | | | | | | | | | | | |
| 48933 | MMP2 | MMP2, | matrix | 0.8 | 1.1 | 1.3 | 1.1 | 1.1 | 1.0 | [symbol] | 1.6 | 1.1 | 1.0 | 4.7 |
| | | gelatinase A | metalloproteinase | | | | | | | | | | | |
| | | | 2 (gelatinase A, | | | | | | | | | | | |
| | | | 72 kD gel | | | | | | | | | | | |
| 50755 | MMP2 | MMP2, | matrix | 0.8 | 1.0 | 1.4 | 1.3 | 1.1 | 1.0 | 1.5 | 1.4 | 1.1 | 1.0 | 4.7 |
| | | gelatinase A | metalloproteinase | | | | | | | | | | | |
| | | | 2 (gelatinase A, | | | | | | | | | | | |
| | | | 72 kD gel | | | | | | | | | | | |
| 47174 | MMP3 | MMP3 | matrix | 3.9 | 3.6 | 1.0 | 1.0 | 0.6 | 1.6 | 1.0 | 1.0 | 0.7 | 1.1 | 4.7 |
| | | | metalloproteinase | | | | | | | | | | | |
| | | | 3 (stromelysin 1, | | | | | | | | | | | |
| | | | progela | | | | | | | | | | | |
| 46630 | MMP3 | MMP3 | matrix | 3.8 | 3.2 | 1.0 | 1.0 | 0.6 | 1.5 | [symbol] | 6.8 | 0.7 | 1.1 | 10.4 |
| | | | metalloproteinase | | | | | | | | | | | |
| | | | 3 (stromelysin 1, | | | | | | | | | | | |
| | | | progela | | | | | | | | | | | |
| 49124 | MMP7 | MMP7 | matrix | 1.4 | 1.3 | 0.4 | 1.5 | 0.9 | 1.0 | [symbol] | 2.1 | 0.9 | 1.0 | 5.6 |
| | | | metalloproteinase | | | | | | | | | | | |
| | | | 7 (matrilysin, | | | | | | | | | | | |
| | | | uterine) | | | | | | | | | | | |
| 48334 | MMP7 | MMP7 | matrix | 1.5 | 1.4 | 0.1 | 4.3 | 0.9 | 1.1 | [symbol] | 4.4 | 0.9 | 1.0 | 10.8 |
| | | | metalloproteinase | | | | | | | | | | | |
| | | | 7 (matrilysin, | | | | | | | | | | | |
| | | | uterine) | | | | | | | | | | | |
| 48187 | MRPL27 | | | 1.1 | 1.0 | 0.7 | 1.4 | 1.0 | 1.0 | 0.6 | 1.2 | 1.0 | 1.0 | 4.6 |
| 47106 | MT1L | | | 12.3 | 10.0 | 0.7 | 1.4 | 0.9 | 1.0 | 0.8 | 1.0 | 1.1 | 1.1 | 4.5 |
| 46766 | MT3 | | | 9.4 | 5.6 | 1.2 | 1.1 | 1.2 | 1.1 | 1.4 | 1.3 | 1.1 | 1.0 | 4.5 |
| 47772 | MTHFD2 | | | 2.8 | 2.7 | 0.8 | 1.1 | 0.9 | 1.1 | 1.0 | 1.0 | 1.1 | 1.0 | 4.2 |
| 48399 | MUC1 | mucin1 | mucin 1, | 2.6 | 2.5 | 1.1 | 1.0 | 0.9 | 1.0 | 0.7 | 1.2 | 0.9 | 1.0 | 4.3 |
| | | | transmembrane | | | | | | | | | | | |
| 50460 | MUC1 | mucin1 | mucin 1, | 3.0 | 2.3 | 1.2 | 1.0 | 1.0 | 1.0 | 0.7 | 1.3 | 0.9 | 1.0 | 4.3 |
| | | | transmembrane | | | | | | | | | | | |
| 50390 | MUC13 | mucin13 | mucin 13, | 2.3 | 2.0 | 1.0 | 1.0 | 1.0 | 1.0 | 1.0 | 1.0 | 0.8 | 1.0 | 4.0 |
| | | | epithelial | | | | | | | | | | | |
| | | | transmembrane | | | | | | | | | | | |
| 50406 | MX1 | | | 13.2 | 10.4 | 1.8 | 1.7 | 1.2 | 1.1 | 1.3 | 1.1 | 1.1 | 1.0 | 4.9 |
| 47450 | MX2 | | | 18.3 | 14.2 | 2.2 | 1.6 | 1.2 | 1.1 | 1.1 | 1.0 | 1.0 | 1.0 | 4.7 |
| 46860 | MYB | c-myb | v-myb avian | 2.4 | 2.1 | 0.8 | 1.0 | 0.9 | 1.0 | 1.1 | 1.0 | 0.9 | 1.0 | 4.0 |
| | | | myeloblastosis | | | | | | | | | | | |
| | | | viral oncogene | | | | | | | | | | | |
| | | | homolog | | | | | | | | | | | |
| 47968 | MYLK | | | 0.8 | 1.2 | 0.6 | 1.4 | 1.0 | 1.0 | 0.7 | 1.3 | 0.9 | 1.0 | 4.7 |
| 47754 | NBS1 | | | 3.7 | 3.0 | 0.7 | 1.4 | 1.1 | 1.0 | 0.7 | 1.3 | 1.2 | 1.1 | 4.7 |
| 48487 | NDN | | | 3.0 | 1.7 | 1.1 | 1.0 | 0.8 | 1.1 | 1.6 | 1.4 | 1.0 | 1.0 | 4.5 |
| 48290 | NEDD4L | | | 1.6 | 1.5 | 0.6 | 1.4 | 0.8 | 1.1 | [symbol] | 1.5 | 0.8 | 1.1 | 5.0 |
| 48866 | NEO1 | | | 1.0 | 1.0 | 1.6 | 1.4 | 1.1 | 1.0 | 1.4 | 1.1 | 1.1 | 1.0 | 4.5 |
| 47584 | NFAT5 | NFAT 5 | nuclear factor of | 3.0 | 2.8 | 0.9 | 1.0 | 0.9 | 1.0 | 1.3 | 1.2 | 1.1 | 1.0 | 4.2 |
| | | | activated T-cells | | | | | | | | | | | |
| | | | 5, tonicity-re | | | | | | | | | | | |
| 50442 | NFATC3 | NFAT 4 | nuclear factor of | 0.5 | 1.9 | 1.6 | 1.4 | 1.0 | 1.0 | 1.2 | 1.0 | 0.9 | 1.1 | 4.5 |
| | | | activated T-cells, | | | | | | | | | | | |
| | | | cytoplasmic, | | | | | | | | | | | |
| 46799 | NFE2L1 | NFE2L1 | nuclear factor | 2.7 | 2.6 | 1.3 | 1.2 | 1.0 | 1.0 | 0.7 | 1.3 | 0.9 | 1.0 | 4.5 |
| | | | (erythroid-derived | | | | | | | | | | | |
| | | | 2)-like 1 | | | | | | | | | | | |
| 47201 | NFIA | NFAT 3 | nuclear factor I/A | 6.8 | 6.2 | 1.0 | 1.0 | 1.0 | 1.0 | 1.0 | 1.0 | 1.0 | 1.0 | 4.0 |
| 47877 | NFKB1 | NFkappaB, | nuclear factor of | 5.7 | 4.3 | 0.7 | 1.2 | 0.9 | 1.1 | 1.2 | 1.1 | 1.1 | 1.0 | 4.5 |
| | | p105 | kappa light | | | | | | | | | | | |
| | | | polypeptide gene | | | | | | | | | | | |
| | | | enh | | | | | | | | | | | |
| 47173 | NFKB1 | NFkappaB, | nuclear factor of | 4.7 | 3.5 | 0.7 | 1.2 | 0.9 | 1.0 | 1.2 | 1.1 | 1.1 | 1.0 | 4.3 |
| | | p105 | kappa light | | | | | | | | | | | |
| | | | polypeptide gene | | | | | | | | | | | |
| | | | enh | | | | | | | | | | | |
| 50676 | NFKB2 | NFkappaB, | nuclear factor of | 3.6 | 2.3 | 1.0 | 1.0 | 1.0 | 1.0 | 1.3 | 1.3 | 1.0 | 1.0 | 4.3 |
| | | p49/p100 | kappa light | | | | | | | | | | | |
| | | | polypeptide gene | | | | | | | | | | | |
| | | | enh | | | | | | | | | | | |
| 50547 | NFKB2 | NFkappaB, | nuclear factor of | 3.5 | 2.3 | 1.0 | 1.0 | 0.9 | 1.0 | 1.3 | 1.2 | 1.0 | 1.0 | 4.2 |
| | | p49/p100 | kappa light | | | | | | | | | | | |
| | | | polypeptide gene | | | | | | | | | | | |
| | | | enh | | | | | | | | | | | |
| 49046 | NFKBIA | IkB, alpha | nuclear factor of | 7.0 | 6.3 | 0.7 | 1.4 | 0.9 | 1.0 | 1.1 | 1.0 | 1.0 | 1.0 | 4.4 |
| | | | kappa light | | | | | | | | | | | |
| | | | polypeptide gene | | | | | | | | | | | |
| | | | enh | | | | | | | | | | | |
| 47056 | NFRKB | | | 0.8 | 1.2 | 0.6 | 1.5 | 0.9 | 1.0 | 0.6 | 1.4 | 0.9 | 1.1 | 5.0 |
| 49690 | NID | NID | nidogen (enactin) | 1.0 | 1.0 | 1.4 | 1.3 | 1.1 | 1.0 | 1.4 | 1.3 | 1.2 | 1.0 | 4.6 |
| 49014 | NINJ1 | | | 5.1 | 5.0 | 0.7 | 1.3 | 1.1 | 1.0 | 0.8 | 1.1 | 1.0 | 1.0 | 4.4 |
| 49872 | NMA | | | 4.3 | 2.6 | 1.6 | 1.2 | 0.9 | 1.0 | [symbol] | 1.6 | 1.1 | 1.1 | 4.9 |
| 48289 | NR1H3 | | | 1.3 | 1.1 | 0.6 | 1.3 | 1.0 | 1.0 | [symbol] | 1.5 | 1.0 | 1.0 | 4.7 |
| 49383 | NR4A1 | NAK1 | nuclear receptor | 3.3 | 2.8 | 1.1 | 1.0 | 1.1 | 1.0 | 1.4 | 1.2 | 1.1 | 1.0 | 4.3 |
| | | | subfamily 4, group | | | | | | | | | | | |
| | | | A, member 1 | | | | | | | | | | | |
| 48941 | NR4A1 | NAK1 | nuclear receptor | 20.4 | 5.2 | 0.8 | 1.0 | 0.7 | 1.1 | 0.7 | 1.0 | 0.9 | 1.0 | 4.1 |
| | | | subfamily 4, group | | | | | | | | | | | |
| | | | A, member 1 | | | | | | | | | | | |
| 47897 | NR4A2 | TINUR | nuclear receptor | 7.0 | 6.0 | 0.9 | 1.0 | 0.9 | 1.0 | 1.8 | 1.1 | 0.8 | 1.0 | 4.1 |
| | | | subfamily 4, group | | | | | | | | | | | |
| | | | A, member 2 | | | | | | | | | | | |
| 47712 | NR4A2 | TINUR | nuclear receptor | 8.1 | 4.9 | 1.1 | 1.0 | 0.9 | 1.0 | 1.4 | 1.1 | 1.2 | 1.0 | 4.1 |
| | | | subfamily 4, group | | | | | | | | | | | |
| | | | A, member 2 | | | | | | | | | | | |
| 47679 | NR4A2 | TINUR | nuclear receptor | 4.8 | 2.6 | 1.2 | 1.0 | 1.0 | 1.0 | 1.3 | 1.2 | 1.1 | 1.0 | 4.2 |
| | | | subfamily 4, group | | | | | | | | | | | |
| | | | A, member2 | | | | | | | | | | | |
| 48227 | NRG1 | neuregulin1, | neuregulin 1 | 5.8 | 3.5 | 0.4 | 1.9 | 0.7 | 1.2 | [symbol] | 1.5 | 0.8 | 1.1 | 5.8 |
| | | neu diff. | | | | | | | | | | | | |
| | | Factor, | | | | | | | | | | | | |
| | | heregulin | | | | | | | | | | | | |
| 48468 | NRG1 | neuregulin1, | neuregulin 1 | 5.3 | 4.1 | 0.5 | 1.6 | 0.7 | 1.3 | 0.7 | 1.0 | 0.8 | 1.1 | 5.1 |
| | | neu diff. | | | | | | | | | | | | |
| | | Factor, | | | | | | | | | | | | |
| | | heregulin | | | | | | | | | | | | |
| 46661 | ORC6L | | | 2.7 | 2.5 | 0.9 | 1.0 | 1.0 | 1.0 | 1.0 | 1.0 | [symbol] | 1.3 | 4.3 |
| 47183 | OSMR | oncostatin MR | oncostatin M | 2.8 | 2.5 | 1.0 | 1.0 | 1.0 | 1.0 | 0.4 | 1.2 | 0.7 | 1.2 | 4.5 |
| | | | receptor | | | | | | | | | | | |
| 49778 | OSMR | oncostatin MR | oncostatin M | 3.2 | 2.6 | 0.8 | 1.0 | 0.9 | 1.0 | 0.4 | 1.4 | 0.7 | 1.0 | 4.4 |
| | | | receptor | | | | | | | | | | | |
| 48707 | P4HA1 | P4HA1, prolyl- | procollagen- | 1.1 | 1.0 | 1.5 | 1.4 | 1.0 | 1.0 | 1.4 | 1.2 | 1.0 | 1.0 | 4.6 |
| | | 4-hydroxylase | proline, 2- | | | | | | | | | | | |
| | | alpha1 | oxoglutarate 4- | | | | | | | | | | | |
| | | | dioxygenase | | | | | | | | | | | |
| 47935 | P4HA1 | P4HA1, prolyl- | procollagen- | 1.2 | 1.0 | 1.5 | 1.4 | 0.9 | 1.0 | 1.7 | 1.2 | 1.0 | 1.0 | 4.7 |
| | | 4-hydroxylase | proline, 2- | | | | | | | | | | | |
| | | alpha1 | oxoglutarate 4- | | | | | | | | | | | |
| | | | dioxygenase | | | | | | | | | | | |
| 48971 | P4HA2 | P4HA2 | procollagen- | 1.2 | 1.1 | 2.0 | 1.7 | 1.0 | 1.0 | 1.7 | 1.4 | 1.0 | 1.0 | 5.1 |
| | | | proline, 2- | | | | | | | | | | | |
| | | | oxoglutarate 4- | | | | | | | | | | | |
| | | | dioxygenase | | | | | | | | | | | |
| 47366 | P4HB | P4HB | procollagen- | 1.0 | 1.0 | 1.4 | 1.4 | 1.0 | 1.0 | 1.4 | 1.4 | 1.1 | 1.1 | 4.8 |
| | | | proline, 2- | | | | | | | | | | | |
| | | | oxoglutarate 4- | | | | | | | | | | | |
| | | | dioxygenase | | | | | | | | | | | |
| 47699 | PAK1 | Pak1 | p21/Cdc42/Rac1- | 0.9 | 1.1 | 2.0 | 1.8 | 1.0 | 1.0 | 1.4 | 1.3 | 1.0 | 1.0 | 5.1 |
| | | | activated kinase 1 | | | | | | | | | | | |
| | | | (yeast Ste20-rel | | | | | | | | | | | |
| 47688 | PAK1 | Pak1 | p21/Cdc42/Rac1- | 0.7 | 1.3 | 1.8 | 1.5 | 1.0 | 1.0 | 1.3 | 1.2 | 0.9 | 1.0 | 4.6 |
| | | | activated kinase 1 | | | | | | | | | | | |
| | | | (yeast Ste20-rel | | | | | | | | | | | |
| 47655 | PAK1 | Pak1 | p21/Cdc42/Rac1- | 0.7 | 1.0 | 1.9 | 1.5 | 1.0 | 1.0 | 1.2 | 1.0 | 0.9 | 1.0 | 4.5 |
| | | | activated kinase 1 | | | | | | | | | | | |
| | | | (yeast Ste20-rel | | | | | | | | | | | |
| 46834 | PAK4 | Pak4 | protein kinase | 2.1 | 1.7 | 0.8 | 1.1 | 1.0 | 1.0 | [symbol] | 1.5 | 1.0 | 1.0 | 4.5 |
| | | | related to S. cerevisiae | | | | | | | | | | | |
| | | | STE20, | | | | | | | | | | | |
| | | | eff | | | | | | | | | | | |
| 49104 | PARG1 | PARG1 | PTPL1-associated | 1.1 | 1.0 | 1.5 | 1.3 | 1.1 | 1.0 | 1.0 | 1.0 | 1.1 | 1.0 | 4.3 |
| | | | RhoGAP 1 | | | | | | | | | | | |
| 48827 | PBX3 | | | 0.9 | 1.0 | 2.1 | 1.8 | 1.1 | 1.0 | [symbol] | 1.8 | 1.1 | 1.0 | 5.7 |
| 47050 | PCDH17 | | | 4.9 | 4.1 | 1.0 | 1.0 | 1.0 | 1.0 | 0.7 | 1.0 | 1.1 | 1.0 | 4.0 |
| 49705 | PCYT2 | | | 0.4 | 2.5 | 1.0 | 1.0 | 1.0 | 1.0 | 1.0 | 1.0 | 0.9 | 1.0 | 4.0 |
| 48556 | PDE4B | phosphodiesterase | phosphodiesterase | 9.8 | 9.4 | 1.0 | 1.0 | 1.1 | 1.0 | 0.8 | 1.1 | 1.1 | 1.1 | 4.1 |
| | | 4B | 4B, cAMP- | | | | | | | | | | | |
| | | | specific (dunce | | | | | | | | | | | |
| | | | (Drosop | | | | | | | | | | | |
| 48427 | PDE4B | phosphodiesterase | phosphodiesterase | 9.4 | 8.8 | 1.0 | 1.0 | 1.0 | 1.0 | 0.8 | 1.1 | 1.1 | 1.1 | 4.2 |
| | | 4B | 4B, cAMP- | | | | | | | | | | | |
| | | | specific (dunce | | | | | | | | | | | |
| | | | (Drosop | | | | | | | | | | | |
| 50597 | PDE7A | | | 0.9 | 1.0 | 1.8 | 1.7 | 1.0 | 1.0 | [symbol] | 1.7 | 0.9 | 1.0 | 5.4 |
| 48370 | PDGFA | PDGF, alpha | platelet-derived | 0.4 | 2.3 | 0.6 | 1.6 | 0.9 | 1.0 | 0.9 | 1.0 | 1.0 | 1.0 | 4.7 |
| | | | growth factor | | | | | | | | | | | |
| | | | alpha polypeptide | | | | | | | | | | | |
| 47014 | PDGFA | PDGF, alpha | platelet-derived | 0.5 | 1.6 | 0.6 | 1.4 | 1.0 | 1.0 | 0.8 | 1.2 | 1.1 | 1.0 | 4.6 |
| | | | growth factor | | | | | | | | | | | |
| | | | alpha polypeptide | | | | | | | | | | | |
| 49625 | PDGFB | PDGF, beta | platelet-derived | 3.4 | 2.2 | 0.9 | 1.0 | 1.0 | 1.0 | 0.7 | 1.2 | 0.9 | 1.0 | 4.2 |
| | | | growth factor beta | | | | | | | | | | | |
| | | | polypeptide (s | | | | | | | | | | | |
| 48824 | PDK2 | | | 0.3 | 2.8 | 1.3 | 1.2 | 1.1 | 1.0 | 1.0 | 1.0 | 1.0 | 1.0 | 4.2 |
| 49745 | PFKP | | | 0.8 | 1.0 | 1.3 | 1.2 | 1.1 | 1.0 | 1.4 | 1.4 | 1.1 | 1.0 | 4.6 |
| 46890 | PHKA1 | | | 0.9 | 1.0 | 1.7 | 1.5 | 1.1 | 1.1 | 1.3 | 1.2 | 1.2 | 1.1 | 4.8 |
| 50038 | PIK3R3 | PI3K | phosphoinositide- | 2.2 | 2.0 | 1.0 | 1.0 | 1.0 | 1.0 | 0.9 | 1.0 | 1.0 | 1.0 | 4.0 |
| | | regulatory | 3-kinase, | | | | | | | | | | | |
| | | subunit, p55 | regulatory subunit, | | | | | | | | | | | |
| | | | pol | | | | | | | | | | | |
| 48759 | PILR(ALPHA) | | | 1.3 | 1.1 | 1.8 | 1.6 | 1.0 | 1.0 | 1.6 | 1.3 | 1.0 | 1.0 | 4.9 |
| 49975 | PIM1 | pim-1 | pim-1 oncogene | 2.4 | 2.0 | 1.1 | 1.0 | 1.1 | 1.1 | 1.8 | 1.4 | 1.1 | 1.0 | 4.5 |
| 46818 | PIM2 | Pim-2 | pim-2 oncogene | 4.8 | 3.3 | 1.1 | 1.0 | 0.9 | 1.0 | [symbol] | 1.7 | 1.1 | 1.1 | 4.8 |
| 50338 | PIM2 | Pim-2 | pim-2 oncogene | 4.7 | 2.8 | 1.3 | 1.2 | 1.0 | 1.0 | [symbol] | 1.8 | 1.1 | 1.0 | 5.0 |
| 48920 | PKM2 | | | 1.0 | 1.0 | 1.3 | 1.2 | 0.9 | 1.0 | 1.5 | 1.3 | 1.0 | 1.0 | 4.6 |
| 47118 | PLA2G4A | phospholipase | phospholipase A2, | 2.0 | 1.6 | 1.3 | 1.2 | 1.0 | 1.0 | [symbol] | 1.6 | [symbol] | 1.3 | 5.1 |
| | | A2 | group IVA | | | | | | | | | | | |
| | | | (cytosolic, | | | | | | | | | | | |
| | | | calcium-de | | | | | | | | | | | |
| 46577 | PLA2G4B | phospholipase | phospholipase A2, | 0.7 | 1.2 | 0.7 | 1.3 | 1.0 | 1.0 | 0.6 | 1.2 | 0.9 | 1.0 | 4.5 |
| | | A2 | group IVB | | | | | | | | | | | |
| | | | (cytosolic) | | | | | | | | | | | |
| 48607 | PLA2G7 | phospholipase | phospholipase A2, | 2.2 | 2.0 | 1.5 | 1.3 | 1.2 | 1.1 | 1.2 | 1.1 | 1.3 | 1.1 | 4.6 |
| | | A2 | group VII (platelet- | | | | | | | | | | | |
| | | | activating f | | | | | | | | | | | |
| 50434 | PLAB | PLAB | prostate | 0.3 | 3.3 | 0.5 | 1.7 | 0.9 | 1.0 | 1.0 | 1.0 | 0.8 | 1.0 | 4.7 |
| | | | differentiation | | | | | | | | | | | |
| | | | factor | | | | | | | | | | | |
| 48023 | PLAB | PLAB | prostate | 0.3 | 3.3 | 0.5 | 1.9 | 0.8 | 1.0 | 0.9 | 1.0 | 0.9 | 1.0 | 4.9 |
| | | | differentiation | | | | | | | | | | | |
| | | | factor | | | | | | | | | | | |
| 49424 | PLAU | urokinase-type | plasminogen | 1.8 | 1.5 | 0.7 | 1.1 | 0.7 | 1.3 | [symbol] | 2.5 | 0.8 | 1.2 | 6.1 |
| | | plasminogen | activator, | | | | | | | | | | | |
| | | activator | urokinase | | | | | | | | | | | |
| 47537 | PLAU | urokinase-type | plasminogen | 1.9 | 1.7 | 0.6 | 1.3 | 0.8 | 1.3 | [symbol] | 2.6 | 0.8 | 1.2 | 6.3 |
| | | plasminogen | activator, | | | | | | | | | | | |
| | | activator | urokinase | | | | | | | | | | | |
| 49422 | PLCG1 | PLC gamma | phospholipase C, | 0.4 | 2.4 | 0.8 | 1.1 | 1.1 | 1.0 | 0.7 | 1.2 | 0.9 | 1.0 | 4.4 |
| | | 1, | gamma 1 | | | | | | | | | | | |
| | | phopholipase | (formerly subtype | | | | | | | | | | | |
| | | C, gamma 1 | 148) | | | | | | | | | | | |
| 50490 | PLD1 | phospholipase | phospholipase D1, | 3.4 | 3.2 | 1.9 | 1.7 | 1.3 | 1.0 | 1.2 | 1.0 | 1.1 | 1.0 | 4.7 |
| | | D1 | phophatidylcholine- | | | | | | | | | | | |
| | | | specific | | | | | | | | | | | |
| 49106 | PLOD | PLOD | procollagen- | 0.6 | 1.7 | 1.9 | 1.7 | 1.0 | 1.0 | [symbol] | 1.6 | 0.9 | 1.0 | 5.3 |
| | | | lysine, 2- | | | | | | | | | | | |
| | | | oxoglutarate 5- | | | | | | | | | | | |
| | | | dioxygenase ( | | | | | | | | | | | |
| 47704 | PLOD | PLOD | procollagen- | 0.6 | 1.5 | 1.9 | 1.7 | 0.9 | 1.0 | [symbol] | 1.7 | 1.0 | 1.0 | 5.4 |
| | | | lysine, 2- | | | | | | | | | | | |
| | | | oxoglutarate 5- | | | | | | | | | | | |
| | | | dioxygenase ( | | | | | | | | | | | |
| 49073 | PLOD | PLOD | procollagen- | 0.7 | 1.4 | 1.9 | 1.7 | 1.0 | 1.0 | [symbol] | 1.6 | 1.0 | 1.0 | 5.3 |
| | | | lysine, 2- | | | | | | | | | | | |
| | | | oxoglutarate 5- | | | | | | | | | | | |
| | | | dioxygenase ( | | | | | | | | | | | |
| 47671 | PLOD | PLOD | procollagen- | 0.6 | 1.5 | 1.9 | 1.7 | 0.9 | 1.0 | [symbol] | 1.6 | 1.1 | 1.0 | 5.3 |
| | | | lysine, 2- | | | | | | | | | | | |
| | | | oxoglutarate 5- | | | | | | | | | | | |
| | | | dioxygenase ( | | | | | | | | | | | |
| 49111 | PLOD3 | PLOD3 | procollagen- | 0.7 | 1.2 | 1.3 | 1.2 | 1.0 | 1.0 | 1.5 | 1.4 | 1.0 | 1.0 | 4.6 |
| | | | lysine, 2- | | | | | | | | | | | |
| | | | oxoglutarate 5- | | | | | | | | | | | |
| | | | dioxygenase 3 | | | | | | | | | | | |
| 48675 | PLOD3 | PLOD3 | procollagen- | 0.9 | 1.0 | 1.3 | 1.2 | 1.0 | 1.0 | 1.5 | 1.4 | 1.1 | 1.0 | 4.5 |
| | | | lysine, 2- | | | | | | | | | | | |
| | | | oxoglutarate 5- | | | | | | | | | | | |
| | | | dioxygenase 3 | | | | | | | | | | | |
| 47419 | PLS3 | | | 1.6 | 1.4 | 0.6 | 1.4 | 0.9 | 1.0 | 0.8 | 1.0 | 1.1 | 1.1 | 4.5 |
| 49809 | PLXNB1 | PLXNB1 | plexin B1 | 0.9 | 1.0 | 0.6 | 1.5 | 0.9 | 1.0 | 0.7 | 1.2 | 0.8 | 1.0 | 4.7 |
| 50073 | PM5 | | | 0.9 | 1.1 | 1.3 | 1.2 | 1.1 | 1.0 | 1.5 | 1.4 | 1.2 | 1.1 | 4.7 |
| 50534 | PPBP | NAP-2 | pro-platelet basic | 1.1 | 1.1 | 0.6 | 1.4 | 0.8 | 1.1 | 0.5 | 1.2 | 0.8 | 1.1 | 4.8 |
| | | | protein (includes | | | | | | | | | | | |
| | | | platelet basi | | | | | | | | | | | |
| 49832 | PPFIBP1 | PPFIBP1 | PTPRF interacting | 0.6 | 1.5 | 0.7 | 1.3 | 0.9 | 1.1 | 0.7 | 1.2 | 0.9 | 1.0 | 4.6 |
| | | | protein, binding | | | | | | | | | | | |
| | | | protein 1 (lipr | | | | | | | | | | | |
| 47328 | PPFIBP1 | PPFIBP1 | PTPRF interacting | 0.6 | 1.5 | 0.6 | 1.6 | 0.9 | 1.0 | 0.7 | 1.3 | 0.9 | 1.0 | 5.0 |
| | | | protein, binding | | | | | | | | | | | |
| | | | protein 1 (lipr | | | | | | | | | | | |
| 49142 | PPIB | cyclophilin B | peptidylprolyl | 0.9 | 1.0 | 1.5 | 1.3 | 1.1 | 1.0 | 1.2 | 1.1 | 1.0 | 1.0 | 4.4 |
| | | | isomerase B | | | | | | | | | | | |
| | | | (cyclophilin B) | | | | | | | | | | | |
| 46983 | PPIC | cyclophilin C | peptidylprolyl | 1.0 | 1.0 | 2.0 | 1.5 | 1.2 | 1.2 | [symbol] | 1.6 | 1.4 | 1.2 | 5.6 |
| | | | isomerase C | | | | | | | | | | | |
| | | | (cyclophilin C) | | | | | | | | | | | |
| 50387 | PPIC | cyclophilin C | peptidylprolyl | 0.9 | 1.0 | 2.0 | 1.9 | 1.2 | 1.1 | [symbol] | 1.9 | 1.2 | 1.0 | 5.9 |
| | | | isomerase C | | | | | | | | | | | |
| | | | (cyclophilin C) | | | | | | | | | | | |
| 46862 | PPIF | cyclophilin F | peptidylprolyl | 1.3 | 1.2 | 0.8 | 1.0 | 0.9 | 1.1 | [symbol] | 1.9 | [symbol] | 1.3 | 5.4 |
| | | | isomerase F | | | | | | | | | | | |
| | | | (cyclophilin F) | | | | | | | | | | | |
| 48616 | PPIH | cyclophilin H | peptidyl prolyl | 0.8 | 1.1 | 0.8 | 1.2 | 0.9 | 1.0 | 0.7 | 1.4 | 0.9 | 1.1 | 4.6 |
| | | | isomerase H | | | | | | | | | | | |
| | | | (cyclophilin H) | | | | | | | | | | | |
| 46624 | PPP3CC | calcineurin A | protein | 3.5 | 2.7 | 0.9 | 1.0 | 1.0 | 1.0 | 1.0 | 1.0 | 1.0 | 1.0 | 4.0 |
| | | gamma | phosphatase 3 | | | | | | | | | | | |
| | | | (formerly 2B), | | | | | | | | | | | |
| | | | catalytic sub | | | | | | | | | | | |
| 49079 | PPP3CC | calcineurin A | protein | 3.2 | 2.4 | 1.0 | 1.0 | 1.0 | 1.0 | 1.1 | 1.0 | 1.0 | 1.0 | 4.0 |
| | | gamma | phosphatase 3 | | | | | | | | | | | |
| | | | (formerly 2B), | | | | | | | | | | | |
| | | | catalytic sub | | | | | | | | | | | |
| 49331 | PRKAR1A | PKA, | protein kinase, | 0.8 | 1.2 | 0.8 | 1.2 | 1.0 | 1.0 | 0.7 | 1.3 | 1.0 | 1.0 | 4.5 |
| | | regulatory | cAMP-dependent, | | | | | | | | | | | |
| | | subunit, type I | regulatory, type I | | | | | | | | | | | |
| | | alpha | | | | | | | | | | | | |
| 47513 | PRKCB1 | PKC, beta | protein kinase C, | 1.0 | 1.0 | 2.0 | 1.8 | 1.1 | 1.0 | [symbol] | 1.7 | 1.2 | 1.1 | 5.5 |
| | | | beta 1 | | | | | | | | | | | |
| 47528 | PRKCN | PKC, nu | protein kinase C, | 1.8 | 1.6 | 0.7 | 1.3 | 0.9 | 1.0 | 0.8 | 1.1 | 0.9 | 1.1 | 4.6 |
| | | | nu | | | | | | | | | | | |
| 49921 | PRKR | | | 4.4 | 4.3 | 1.8 | 1.5 | 1.1 | 1.0 | 1.3 | 1.2 | 1.2 | 1.1 | 4.7 |
| 50643 | PROS1 | | | 0.9 | 1.0 | 1.5 | 1.4 | 1.0 | 1.0 | 1.5 | 1.4 | 1.1 | 1.0 | 4.8 |
| 49006 | PRSS25 | | | 1.0 | 1.0 | 1.4 | 1.4 | 1.1 | 1.0 | 1.2 | 1.1 | 1.0 | 1.0 | 4.5 |
| 47998 | PSMB9 | | | 4.3 | 3.6 | 1.4 | 1.3 | 1.2 | 1.1 | 0.9 | 1.0 | 1.1 | 1.0 | 4.4 |
| 47690 | PTEN | PTEN | phosphatase and | 1.9 | 1.7 | 1.6 | 1.5 | 1.0 | 1.0 | 1.3 | 1.2 | 1.1 | 1.0 | 4.7 |
| | | | tensin homolog | | | | | | | | | | | |
| | | | (mutated in multipl | | | | | | | | | | | |
| 49846 | PTEN | PTEN | phosphatase and | 1.2 | 1.1 | 1.3 | 1.2 | 1.0 | 1.0 | 1.4 | 1.4 | 1.1 | 1.0 | 4.6 |
| | | | tensin homolog | | | | | | | | | | | |
| | | | (mutated in multipl | | | | | | | | | | | |
| 47657 | PTEN | PTEN | phosphatase and | 3.9 | 3.1 | 1.2 | 1.1 | 0.9 | 1.0 | 1.1 | 1.0 | 1.0 | 1.0 | 4.1 |
| | | | tensin homolog | | | | | | | | | | | |
| | | | (mutated in multipl | | | | | | | | | | | |
| 47198 | PTGER2 | PTGER2 | prostaglandin E | 5.3 | 4.7 | 1.0 | 1.0 | 1.0 | 1.0 | 1.4 | 1.1 | 1.0 | 1.0 | 4.1 |
| | | | receptor 2 | | | | | | | | | | | |
| | | | (subtype EP2), | | | | | | | | | | | |
| | | | 53 kD | | | | | | | | | | | |
| 46654 | PTGER2 | PTGER2 | prostaglandin E | 4.0 | 3.1 | 1.0 | 1.0 | 0.9 | 1.0 | 1.2 | 1.0 | 1.0 | 1.0 | 4.0 |
| | | | receptor 2 | | | | | | | | | | | |
| | | | (subtype EP2), | | | | | | | | | | | |
| | | | 53 kD | | | | | | | | | | | |
| 48422 | PTGER2 | PTGER2 | prostaglandin E | 3.3 | 2.8 | 0.8 | 1.0 | 1.0 | 1.0 | 1.0 | 1.0 | 1.0 | 1.0 | 4.0 |
| | | | receptor 2 | | | | | | | | | | | |
| | | | (subtype EP2), | | | | | | | | | | | |
| | | | 53 kD | | | | | | | | | | | |
| 47551 | PTGER4 | EP2 | prostaglandin E | 1.4 | 1.3 | 0.7 | 1.3 | 1.1 | 1.0 | [symbol] | 1.5 | [symbol] | 1.3 | 5.1 |
| | | | receptor 4 | | | | | | | | | | | |
| | | | (subtype EP4) | | | | | | | | | | | |
| 50200 | PTGER4 | EP2 | prostaglandin E | 1.4 | 1.0 | 0.7 | 1.3 | 1.1 | 1.1 | [symbol] | 1.6 | 1.3 | 1.2 | 5.1 |
| | | | receptor 4 | | | | | | | | | | | |
| | | | (subtype EP4) | | | | | | | | | | | |
| 50010 | PTGIR | PTGIR | prostaglandin I2 | 2.8 | 2.7 | 0.8 | 1.1 | 0.9 | 1.1 | 0.8 | 1.2 | 0.8 | 1.1 | 4.4 |
| | | | (prostacyclin) | | | | | | | | | | | |
| | | | receptor (IP) | | | | | | | | | | | |
| 49466 | PTGIR | PTGIR | prostaglandin I2 | 2.5 | 2.2 | 0.8 | 1.0 | 0.9 | 1.0 | 0.8 | 1.1 | 0.9 | 1.0 | 4.2 |
| | | | (prostacyclin) | | | | | | | | | | | |
| | | | receptor (IP) | | | | | | | | | | | |
| 48809 | PTGS1 | PTGS1 | prostaglandin- | 0.3 | 2.9 | 0.6 | 1.4 | 1.0 | 1.0 | [symbol] | 1.5 | 0.9 | 1.0 | 4.9 |
| | | | endoperoxide | | | | | | | | | | | |
| | | | synthase 1 | | | | | | | | | | | |
| | | | (prostagland | | | | | | | | | | | |
| 48625 | PTGS1 | PTGS1 | prostaglandin- | 0.6 | 1.5 | 1.4 | 1.3 | 1.0 | 1.0 | [symbol] | 1.5 | 1.1 | 1.0 | 4.9 |
| | | | endoperoxide | | | | | | | | | | | |
| | | | synthase 1 | | | | | | | | | | | |
| | | | (prostagland | | | | | | | | | | | |
| 47499 | PTGS2 | PTGS2, COX2 | prostaglandin- | 28.8 | 28.0 | 0.7 | 1.0 | 0.9 | 1.0 | [symbol] | 1.9 | 1.3 | 1.1 | 5.1 |
| | | | endoperoxide | | | | | | | | | | | |
| | | | synthase 2 | | | | | | | | | | | |
| | | | (prostagland | | | | | | | | | | | |
| 50665 | PTGS2 | PTGS2, COX2 | prostaglandin- | 32.8 | 31.2 | 1.0 | 1.0 | 0.8 | 1.2 | [symbol] | 2.3 | 1.3 | 1.1 | 5.6 |
| | | | endoperoxide | | | | | | | | | | | |
| | | | synthase 2 | | | | | | | | | | | |
| | | | (prostagland | | | | | | | | | | | |
| 49036 | PTK2B | PYK2 | protein tyrosine | 1.1 | 1.0 | 1.5 | 1.4 | 1.0 | 1.0 | 1.3 | 1.2 | 0.9 | 1.0 | 4.6 |
| | | | kinase 2 beta | | | | | | | | | | | |
| 47997 | PTPN3 | PTPN3 | protein tyrosine | 1.5 | 1.4 | 1.6 | 1.5 | 1.1 | 1.1 | 1.2 | 1.1 | 1.2 | 1.1 | 4.7 |
| | | | phosphatase, non- | | | | | | | | | | | |
| | | | receptor type 3 | | | | | | | | | | | |
| 48940 | PTPRC | CD45 | protein tyrosine | 1.0 | 1.0 | 1.6 | 1.5 | 1.1 | 1.1 | 1.2 | 1.0 | 1.1 | 1.1 | 4.8 |
| | | | phosphatase, | | | | | | | | | | | |
| | | | receptor type, C | | | | | | | | | | | |
| 48685 | PTPRC | CD45 | protein tyrosine | 1.1 | 1.0 | 1.7 | 1.5 | 1.2 | 1.1 | 1.3 | 1.0 | 1.2 | 1.1 | 4.7 |
| | | | phosphatase, | | | | | | | | | | | |
| | | | receptor type, C | | | | | | | | | | | |
| 47308 | PTPRJ | PTPRJ | protein tyrosine | 1.7 | 1.4 | 1.0 | 1.0 | 1.0 | 1.0 | 1.5 | 1.4 | 1.1 | 1.0 | 4.4 |
| | | | phosphatase, | | | | | | | | | | | |
| | | | receptor type, J | | | | | | | | | | | |
| 50632 | PTPRJ | PTPRJ | protein tyrosine | 1.5 | 1.3 | 1.2 | 1.0 | 1.0 | 1.0 | [symbol] | 1.5 | 1.1 | 1.0 | 4.6 |
| | | | phosphatase, | | | | | | | | | | | |
| | | | receptor type, J | | | | | | | | | | | |
| 47698 | PTPRJ | PTPRJ | protein tyrosine | 1.7 | 1.3 | 1.0 | 1.0 | 1.0 | 1.0 | 1.5 | 1.3 | 1.0 | 1.0 | 4.3 |
| | | | phosphatase, | | | | | | | | | | | |
| | | | receptor type, J | | | | | | | | | | | |
| 47548 | PTPRK | PTPRK | protein tyrosine | 3.8 | 3.1 | 0.7 | 1.2 | 0.8 | 1.1 | 1.1 | 1.0 | 1.1 | 1.0 | 4.3 |
| | | | phosphatase, | | | | | | | | | | | |
| | | | receptor type, K | | | | | | | | | | | |
| 48687 | PTPRM | PTPRM | protein tyrosine | 3.1 | 2.7 | 0.8 | 1.0 | 0.9 | 1.0 | 1.0 | 1.0 | 0.9 | 1.0 | 4.1 |
| | | | phosphatase, | | | | | | | | | | | |
| | | | receptor type, M | | | | | | | | | | | |
| 49684 | PTPRO | PTPRO | protein tyrosine | 0.5 | 1.3 | 1.7 | 1.6 | 1.1 | 1.0 | 1.0 | 1.0 | 1.0 | 1.0 | 4.6 |
| | | | phosphatase, | | | | | | | | | | | |
| | | | receptor type, O | | | | | | | | | | | |
| 46748 | PTX3 | | | 8.7 | 3.4 | 1.4 | 1.3 | 1.0 | 1.0 | [symbol] | 1.5 | [symbol] | 1.3 | 5.2 |
| 49876 | RAD54L | | | 0.9 | 1.0 | 0.6 | 1.1 | 1.0 | 1.0 | 0.6 | 1.3 | 1.0 | 1.0 | 4.5 |
| 47446 | RANBP1 | | | 0.8 | 1.1 | 0.6 | 1.4 | 0.9 | 1.0 | 0.7 | 1.2 | 1.0 | 1.0 | 4.6 |
| 47231 | RASA1 | RasGAP | RAS p21 protein | 0.4 | 2.4 | 1.0 | 1.0 | 1.0 | 1.0 | 0.8 | 1.2 | 0.9 | 1.0 | 4.2 |
| | | | activator (GTPase | | | | | | | | | | | |
| | | | activating prote | | | | | | | | | | | |
| 50122 | RASA1 | RasGAP | RAS p21 protein | 0.4 | 2.6 | 1.1 | 1.0 | 1.1 | 1.0 | 1.0 | 1.0 | 1.0 | 1.0 | 4.0 |
| | | | activator (GTPase | | | | | | | | | | | |
| | | | activating prote | | | | | | | | | | | |
| 48031 | RASA2 | RasGAP | RAS p21 protein | 1.4 | 1.3 | 1.0 | 1.0 | 0.9 | 1.0 | [symbol] | 1.5 | 0.9 | 1.0 | 4.5 |
| | | | activator 2 | | | | | | | | | | | |
| 47707 | RASA2 | RasGAP | RAS p21 protein | 1.4 | 1.2 | 1.0 | 1.0 | 0.9 | 1.0 | [symbol] | 1.5 | 0.9 | 1.0 | 4.5 |
| | | | activator 2 | | | | | | | | | | | |
| 50244 | RBL1 | | | 2.6 | 2.3 | 1.0 | 1.0 | 1.1 | 1.0 | 1.2 | 1.0 | 1.2 | 1.0 | 4.0 |
| 48471 | RBM3 | | | 1.1 | 1.0 | 0.7 | 1.5 | 1.0 | 1.0 | 0.8 | 1.2 | 1.1 | 1.1 | 4.7 |
| 48843 | RBMS1 | | | 1.3 | 1.2 | 0.9 | 1.1 | 1.1 | 1.0 | [symbol] | 1.5 | 1.0 | 1.0 | 4.6 |
| 48132 | RCN2 | | | 0.8 | 1.2 | 1.4 | 1.2 | 1.0 | 1.0 | 1.4 | 1.4 | 1.0 | 1.0 | 4.6 |
| 49957 | REL | c-rel | v-rel avian | 4.4 | 2.2 | 1.0 | 1.0 | 0.9 | 1.1 | 1.4 | 1.1 | 1.1 | 1.0 | 4.2 |
| | | | reticuloendotheliosis | | | | | | | | | | | |
| | | | viral oncogene h | | | | | | | | | | | |
| 47672 | RELA | p65, NFKB3 | v-rel avian | 2.6 | 2.5 | 0.8 | 1.2 | 0.9 | 1.0 | 1.0 | 1.0 | 1.1 | 1.1 | 4.3 |
| | | | reticuloendotheliosis | | | | | | | | | | | |
| | | | viral oncogene h | | | | | | | | | | | |
| 47652 | RELA | p65, NFKB3 | v-rel avian | 2.4 | 2.1 | 0.8 | 1.2 | 1.0 | 1.0 | 1.0 | 1.0 | 1.1 | 1.0 | 4.2 |
| | | | reticuloendotheliosis | | | | | | | | | | | |
| | | | viral oncogene h | | | | | | | | | | | |
| 49120 | RELB | I-rel | v-rel avian | 2.5 | 2.2 | 0.7 | 1.2 | 1.0 | 1.0 | 1.0 | 1.0 | 1.0 | 1.0 | 4.2 |
| | | | reticuloendotheliosis | | | | | | | | | | | |
| | | | viral oncogene h | | | | | | | | | | | |
| 49087 | RELB | I-rel | v-rel avian | 2.5 | 2.1 | 0.7 | 1.2 | 1.0 | 1.0 | 1.0 | 1.0 | 1.0 | 1.0 | 4.2 |
| | | | reticuloendotheliosis | | | | | | | | | | | |
| | | | viral oncogene h | | | | | | | | | | | |
| 50499 | RGS14 | RGS14 | regulator of G- | 0.4 | 2.3 | 2.0 | 1.7 | 1.3 | 1.2 | 1.4 | 1.3 | 1.0 | 1.0 | 5.2 |
| | | | protein signalling | | | | | | | | | | | |
| | | | 14 | | | | | | | | | | | |
| 49813 | RGS14 | RGS14 | regulator of G- | 0.3 | 2.4 | 1.7 | 1.2 | 1.3 | 1.2 | 1.1 | 1.0 | 1.1 | 1.0 | 4.4 |
| | | | protein signalling | | | | | | | | | | | |
| | | | 14 | | | | | | | | | | | |
| 49786 | RGS16 | Rgs-16 | regulator of G- | 4.0 | 3.4 | 0.2 | 4.1 | 0.7 | 1.4 | [symbol] | 2.1 | 0.8 | 1.2 | 8.8 |
| | | | protein signalling | | | | | | | | | | | |
| | | | 16 | | | | | | | | | | | |
| 48784 | RGS16 | Rgs-16 | regulator of G- | 3.5 | 3.2 | 0.4 | 2.0 | 0.7 | 1.3 | [symbol] | 2.1 | 0.9 | 1.0 | 6.4 |
| | | | protein signalling | | | | | | | | | | | |
| | | | 16 | | | | | | | | | | | |
| 47933 | RIPK2 | RIP2, RICK | receptor- | 8.5 | 6.4 | 0.9 | 1.1 | 0.9 | 1.0 | 0.9 | 1.0 | 1.1 | 1.0 | 4.1 |
| | | | interacting serine- | | | | | | | | | | | |
| | | | threonine kinase 2 | | | | | | | | | | | |
| 46938 | ROCK2 | ROCK2 | Rho-associated, | 1.4 | 1.0 | 0.8 | 1.2 | 1.0 | 1.0 | 0.7 | 1.4 | 1.0 | 1.0 | 4.6 |
| | | | coiled-coil | | | | | | | | | | | |
| | | | containing protein | | | | | | | | | | | |
| | | | kin | | | | | | | | | | | |
| 50344 | RPS6KA4 | RPS6KA4 | ribosomal protein | 0.4 | 2.1 | 1.2 | 1.1 | 1.1 | 1.0 | 1.0 | 1.0 | 1.0 | 1.0 | 4.1 |
| | | | S6 kinase, 90 kD, | | | | | | | | | | | |
| | | | polypeptide 4 | | | | | | | | | | | |
| 50354 | RPS6KC1 | RPS6KC1 | ribosomal protein | 1.8 | 1.8 | 1.1 | 1.0 | 1.0 | 1.0 | [symbol] | 1.5 | 1.1 | 1.0 | 4.5 |
| | | | S6 kinase, 52 kD, | | | | | | | | | | | |
| | | | polypeptide 1 | | | | | | | | | | | |
| 47224 | RPS6KC1 | RPS6KC1 | ribosomal protein | 2.1 | 1.7 | 1.1 | 1.1 | 0.9 | 1.0 | 1.5 | 1.3 | 1.1 | 1.0 | 4.4 |
| | | | S6 kinase, 52 kD, | | | | | | | | | | | |
| | | | polypeptide 1 | | | | | | | | | | | |
| 49416 | S100A10 | S100A10 | S100 calcium- | 1.2 | 1.0 | 1.9 | 1.5 | 1.1 | 1.0 | 1.9 | 1.4 | 1.2 | 1.1 | 5.1 |
| | | | binding protein | | | | | | | | | | | |
| | | | A10 (annexin II | | | | | | | | | | | |
| | | | ligan | | | | | | | | | | | |
| 50130 | S100A10 | S100A10 | S100 calcium- | 1.2 | 1.1 | 1.9 | 1.5 | 1.1 | 1.0 | 1.7 | 1.4 | 1.1 | 1.0 | 4.8 |
| | | | binding protein | | | | | | | | | | | |
| | | | A10 (annexin II | | | | | | | | | | | |
| | | | ligan | | | | | | | | | | | |
| 50120 | S100A4 | S100A4 | S100 calcium- | 1.0 | 1.0 | 2.2 | 2.0 | 1.2 | 1.1 | [symbol] | 1.9 | 1.2 | 1.1 | 6.2 |
| | | | binding protein A4 | | | | | | | | | | | |
| | | | (calcium protein, | | | | | | | | | | | |
| 47318 | S100A4 | S100A4 | S100 calcium- | 1.0 | 1.0 | 2.0 | 1.8 | 1.2 | 1.0 | [symbol] | 1.6 | 1.2 | 1.1 | 5.6 |
| | | | binding protein A4 | | | | | | | | | | | |
| | | | (calcium protein, | | | | | | | | | | | |
| 50144 | S100A6 | S100A6 | S100 calcium- | 0.9 | 1.0 | 1.4 | 1.3 | 1.2 | 1.1 | 1.5 | 1.1 | 1.1 | 1.0 | 4.5 |
| | | | binding protein A6 | | | | | | | | | | | |
| | | | (calcyclin) | | | | | | | | | | | |
| 50138 | S100A6 | S100A6 | S100 calcium- | 1.0 | 1.0 | 1.4 | 1.3 | 1.1 | 1.1 | 1.5 | 1.0 | 1.1 | 1.0 | 4.4 |
| | | | binding protein A6 | | | | | | | | | | | |
| | | | (calcyclin) | | | | | | | | | | | |
| 49076 | SARM | KIAA0524 | KIAA0524 protein | 1.0 | 1.0 | 1.3 | 1.2 | 0.6 | 1.4 | 1.8 | 1.0 | 0.8 | 1.0 | 4.6 |
| 50441 | SAT | | | 7.2 | 5.4 | 1.0 | 1.0 | 0.9 | 1.0 | 0.9 | 1.0 | 0.8 | 1.1 | 4.1 |
| 49261 | SCYA1 | I-309 | small inducible | 2.9 | 2.7 | 0.8 | 1.0 | 1.0 | 1.0 | 0.8 | 1.1 | 1.1 | 1.0 | 4.1 |
| | | | cytokine A1 (I- | | | | | | | | | | | |
| | | | 309, homologous | | | | | | | | | | | |
| | | | to | | | | | | | | | | | |
| 47503 | SCYA1 | I-309 | small inducible | 2.7 | 2.3 | 1.0 | 1.0 | 1.2 | 1.0 | 0.9 | 1.0 | 1.1 | 1.0 | 4.0 |
| | | | cytokine A1 (I- | | | | | | | | | | | |
| | | | 309, homologous | | | | | | | | | | | |
| | | | to | | | | | | | | | | | |
| 49412 | SCYA15 | MIP-5 | small inducible | 2.4 | 2.2 | 1.0 | 1.0 | 1.1 | 1.0 | 1.2 | 1.1 | 1.1 | 1.0 | 4.1 |
| | | | cytokine subfamily | | | | | | | | | | | |
| | | | A (Cys—Cys), me | | | | | | | | | | | |
| 49044 | SCYA18 | DC-CK1 | small inducible | 26.3 | 23.0 | 0.4 | 1.7 | 0.6 | 1.4 | 1.3 | 1.1 | 1.0 | 1.0 | 5.2 |
| | | | cytokine subfamily | | | | | | | | | | | |
| | | | A (Cys—Cys), me | | | | | | | | | | | |
| 48001 | SCYA18 | DC-CK1 | small inducible | 31.7 | 24.7 | 0.5 | 1.4 | 0.7 | 1.3 | 1.2 | 1.1 | 1.0 | 1.0 | 4.8 |
| | | | cytokine subfamily | | | | | | | | | | | |
| | | | A (Cys—Cys), me | | | | | | | | | | | |
| 48830 | SCYA2 | MCP1 | small inducible | 8.2 | 5.2 | 0.7 | 1.3 | 1.0 | 1.0 | 0.7 | 1.1 | 1.1 | 1.0 | 4.4 |
| | | | cytokine A2 | | | | | | | | | | | |
| | | | (monocyte | | | | | | | | | | | |
| | | | chemotactic | | | | | | | | | | | |
| 47916 | SCYA2 | MCP1 | small inducible | 16.9 | 10.7 | 0.3 | 2.1 | 1.2 | 1.1 | 0.7 | 1.0 | 1.1 | 1.0 | 5.2 |
| | | | cytokine A2 | | | | | | | | | | | |
| | | | (monocyte | | | | | | | | | | | |
| | | | chemotactic | | | | | | | | | | | |
| 50026 | SCYA2 | MCP1 | small inducible | 24.1 | 13.0 | 0.4 | 2.4 | 1.2 | 1.0 | 0.6 | 1.3 | 1.1 | 1.0 | 5.8 |
| | | | cytokine A2 | | | | | | | | | | | |
| | | | (monocyte | | | | | | | | | | | |
| | | | chemotactic | | | | | | | | | | | |
| 46598 | SCYA20 | MIP-3a | small inducible | 8.6 | 7.8 | 0.2 | 4.0 | 0.7 | 1.4 | 0.6 | 1.3 | 1.0 | 1.0 | 7.7 |
| | | | cytokine subfamily | | | | | | | | | | | |
| | | | A (Cys—Cys), me | | | | | | | | | | | |
| 48190 | SCYA20 | MIP-3a | small inducible | 8.1 | 5.6 | 0.1 | 6.1 | 0.7 | 1.4 | 0.6 | 1.0 | 1.1 | 1.0 | 9.5 |
| | | | cytokine subfamily | | | | | | | | | | | |
| | | | A (Cys—Cys), me | | | | | | | | | | | |
| 46596 | SCYA20 | MIP-3a | small inducible | 3.7 | 3.2 | 0.6 | 1.5 | 0.9 | 1.0 | 0.6 | 1.0 | 1.0 | 1.0 | 4.5 |
| | | | cytokine subfamily | | | | | | | | | | | |
| | | | A (Cys—Cys), me | | | | | | | | | | | |
| 46945 | SCYA28 | SCYA28 | CC chemokine | 1.3 | 1.0 | 0.5 | 1.4 | 1.2 | 1.0 | 1.0 | 1.0 | 1.0 | 1.0 | 4.4 |
| | | | CCL28 | | | | | | | | | | | |
| 50009 | SCYA3 | MIP1, alpha | small inducible | 7.8 | 5.0 | 0.4 | 1.8 | 0.8 | 1.0 | 1.2 | 1.2 | 1.2 | 1.1 | 5.1 |
| | | homolog | cytokine A3 | | | | | | | | | | | |
| | | | (homologous to | | | | | | | | | | | |
| | | | mouse M | | | | | | | | | | | |
| 49742 | SCYA3 | MIP1, alpha | small inducible | 9.0 | 8.7 | 0.4 | 1.6 | 0.8 | 1.0 | 1.2 | 1.2 | 1.2 | 1.0 | 4.8 |
| | | homolog | cytokine A3 | | | | | | | | | | | |
| | | | (homologous to | | | | | | | | | | | |
| | | | mouse M | | | | | | | | | | | |
| 50369 | SCYA3 | MIP1, alpha | small inducible | 8.5 | 8.4 | 0.4 | 2.0 | 0.8 | 1.0 | 1.2 | 1.1 | 1.2 | 1.0 | 5.2 |
| | | homolog | cytokine A3 | | | | | | | | | | | |
| | | | (homologous to | | | | | | | | | | | |
| | | | mouse M | | | | | | | | | | | |
| 48585 | SCYA3 | MIP1, alpha | small inducible | 9.5 | 8.6 | 0.5 | 1.8 | 0.8 | 1.1 | 1.2 | 1.2 | 1.2 | 1.0 | 5.1 |
| | | homolog | cytokine A3 | | | | | | | | | | | |
| | | | (homologous to | | | | | | | | | | | |
| | | | mouse M | | | | | | | | | | | |
| 47893 | SCYA4 | MIP-1beta | small inducible | 48.7 | 24.7 | 0.3 | 2.8 | 0.8 | 1.0 | 1.2 | 1.2 | 1.2 | 1.1 | 6.0 |
| | | | cytokine A4 | | | | | | | | | | | |
| | | | (homologous to | | | | | | | | | | | |
| | | | mouse M | | | | | | | | | | | |
| 48257 | SCYA4 | MIP-1beta | small inducible | 40.5 | 17.9 | 0.3 | 1.8 | 0.9 | 1.0 | 1.2 | 1.2 | 1.2 | 1.0 | 5.0 |
| | | | cytokine A4 | | | | | | | | | | | |
| | | | (homologous to | | | | | | | | | | | |
| | | | mouse M | | | | | | | | | | | |
| 47575 | SCYA8 | MCP-2 | small inducible | 47.8 | 46.5 | 1.0 | 1.0 | 1.1 | 1.0 | 0.8 | 1.0 | 1.3 | 1.1 | 4.1 |
| | | | cytokine subfamily | | | | | | | | | | | |
| | | | A (Cys—Cys), me | | | | | | | | | | | |
| 48338 | SCYB11 | I-TAC | small inducible | 38.0 | 37.8 | 0.8 | 1.0 | 0.9 | 1.1 | 1.5 | 1.1 | 1.4 | 1.3 | 4.5 |
| | | | cytokine subfamily | | | | | | | | | | | |
| | | | B (Cys-X-Cys), | | | | | | | | | | | |
| 47726 | SCYB11 | I-TAC | small inducible | 38.7 | 37.6 | 1.2 | 1.0 | 1.0 | 1.0 | 1.5 | 1.0 | [symbol] | 1.3 | 4.3 |
| | | | cytokine subfamily | | | | | | | | | | | |
| | | | B (Cys-X-Cys), | | | | | | | | | | | |
| 49968 | SCYB5 | ENA-78 | small inducible | 2.6 | 2.0 | 0.7 | 1.1 | 0.8 | 1.0 | 0.6 | 1.2 | [symbol] | 1.5 | 4.8 |
| | | | cytokine subfamily | | | | | | | | | | | |
| | | | B (Cys-X-Cys), | | | | | | | | | | | |
| 49839 | SCYB5 | ENA-78 | small inducible | 2.6 | 1.9 | 0.8 | 1.2 | 0.8 | 1.0 | 0.5 | 1.3 | [symbol] | 1.4 | 5.0 |
| | | | cytokine subfamily | | | | | | | | | | | |
| | | | B (Cys-X-Cys), | | | | | | | | | | | |
| 50117 | SDBCAG84 | | | 0.9 | 1.0 | 1.4 | 1.3 | 1.0 | 1.0 | 1.4 | 1.4 | 1.0 | 1.0 | 4.6 |
| 47729 | SELPLG | selectin P | selectin P ligand | 1.1 | 1.0 | 1.5 | 1.4 | 1.1 | 1.0 | 1.1 | 1.0 | 1.1 | 1.1 | 4.5 |
| | | ligand | | | | | | | | | | | | |
| 47858 | SELPLG | selectin P | selectin P ligand | 1.0 | 1.0 | 1.4 | 1.3 | 1.1 | 1.0 | 1.1 | 1.0 | 1.1 | 1.1 | 4.4 |
| | | ligand | | | | | | | | | | | | |
| 49117 | SERPINB1 | | serine (or | 3.8 | 2.9 | 0.7 | 1.2 | 1.0 | 1.0 | [symbol] | 1.9 | 1.2 | 1.0 | 5.2 |
| | | | cysteine) | | | | | | | | | | | |
| | | | proteinase | | | | | | | | | | | |
| | | | inhibitor, clade B | | | | | | | | | | | |
| 46682 | SERPINB2 | | serine (or | 4.2 | 3.4 | 1.0 | 1.0 | 1.1 | 1.0 | 0.6 | 1.3 | 0.8 | 1.2 | 4.5 |
| | | | cysteine) | | | | | | | | | | | |
| | | | proteinase | | | | | | | | | | | |
| | | | inhibitor, clade B | | | | | | | | | | | |
| 48269 | SERPINE1 | | serine (or | 1.1 | 1.0 | 0.6 | 1.3 | 0.7 | 1.2 | 0.7 | 1.0 | 0.7 | 1.2 | 4.7 |
| | | | cysteine) | | | | | | | | | | | |
| | | | proteinase | | | | | | | | | | | |
| | | | inhibitor, clade E | | | | | | | | | | | |
| 48281 | SERPINE2 | | serine (or | 34.6 | 17.3 | 0.4 | 2.2 | 1.0 | 1.0 | 0.6 | 1.1 | 1.1 | 1.0 | 5.3 |
| | | | cysteine) | | | | | | | | | | | |
| | | | proteinase | | | | | | | | | | | |
| | | | inhibitor, clade E | | | | | | | | | | | |
| 48400 | SERPINE2 | | serine (or | 26.7 | 13.2 | 0.4 | 2.2 | 1.0 | 1.0 | 0.7 | 1.1 | 1.0 | 1.0 | 5.3 |
| | | | cysteine) | | | | | | | | | | | |
| | | | proteinase | | | | | | | | | | | |
| | | | inhibitor, clade E | | | | | | | | | | | |
| 48367 | SERPINE2 | | serine (or | 27.1 | 12.5 | 0.4 | 2.1 | 1.0 | 1.0 | 0.7 | 1.2 | 1.1 | 1.0 | 5.3 |
| | | | cysteine) | | | | | | | | | | | |
| | | | proteinase | | | | | | | | | | | |
| | | | inhibitor, clade E | | | | | | | | | | | |
| 48609 | SERPINF1 | | serine (or | 0.8 | 1.1 | 1.5 | 1.3 | 1.1 | 1.0 | [symbol] | 1.7 | 1.1 | 1.0 | 5.0 |
| | | | cysteine) | | | | | | | | | | | |
| | | | proteinase | | | | | | | | | | | |
| | | | inhibitor, clade F | | | | | | | | | | | |
| 49679 | SERPINF1 | | serine (or | 0.8 | 1.2 | 1.6 | 1.4 | 1.1 | 1.1 | [symbol] | 1.5 | 1.1 | 1.0 | 5.0 |
| | | | cysteine) | | | | | | | | | | | |
| | | | proteinase | | | | | | | | | | | |
| | | | inhibitor, clade F | | | | | | | | | | | |
| 48309 | SERPINF1 | | serine (or | 0.9 | 1.1 | 1.5 | 1.5 | 1.1 | 1.0 | 1.6 | 1.3 | 1.1 | 1.0 | 4.8 |
| | | | cysteine) | | | | | | | | | | | |
| | | | proteinase | | | | | | | | | | | |
| | | | inhibitor, clade F | | | | | | | | | | | |
| 46579 | SERPING1 | | serine (or | 2.7 | 2.5 | 0.8 | 1.1 | 1.0 | 1.0 | 0.7 | 1.3 | 1.0 | 1.0 | 4.4 |
| | | | cysteine) | | | | | | | | | | | |
| | | | proteinase | | | | | | | | | | | |
| | | | inhibitor, clade G | | | | | | | | | | | |
| 50083 | SERPING1 | | serine (or | 11.1 | 7.2 | 1.3 | 1.1 | 1.1 | 1.0 | 0.7 | 1.2 | 1.1 | 1.0 | 4.2 |
| | | | cysteine) | | | | | | | | | | | |
| | | | proteinase | | | | | | | | | | | |
| | | | inhibitor, clade G | | | | | | | | | | | |
| 46621 | SERPINH2 | | serine (or | 0.7 | 1.1 | 0.8 | 1.2 | 1.0 | 1.0 | 0.6 | 1.4 | 0.9 | 1.0 | 4.5 |
| | | | cysteine) | | | | | | | | | | | |
| | | | proteinase | | | | | | | | | | | |
| | | | inhibitor, clade H | | | | | | | | | | | |
| 47577 | SERPINH2 | | serine (or | 1.0 | 1.0 | 0.9 | 1.0 | 0.9 | 1.0 | 0.7 | 1.4 | 0.9 | 1.0 | 4.4 |
| | | | cysteine) | | | | | | | | | | | |
| | | | proteinase | | | | | | | | | | | |
| | | | inhibitor, clade H | | | | | | | | | | | |
| 46787 | SERPINI1 | | serine (or | 1.1 | 1.0 | 2.6 | 1.8 | 1.1 | 1.1 | [symbol] | 1.6 | 1.1 | 1.1 | 5.5 |
| | | | cysteine) | | | | | | | | | | | |
| | | | proteinase | | | | | | | | | | | |
| | | | inhibitor, clade I | | | | | | | | | | | |
| 47139 | SERPINI1 | | serine (or | 1.0 | 1.0 | 2.2 | 1.4 | 1.1 | 1.1 | [symbol] | 1.6 | 1.1 | 1.0 | 5.1 |
| | | | cysteine) | | | | | | | | | | | |
| | | | proteinase | | | | | | | | | | | |
| | | | inhibitor, clade I | | | | | | | | | | | |
| 49880 | SLC25A13 | | | 0.8 | 1.2 | 0.7 | 1.2 | 0.9 | 1.0 | 0.7 | 1.3 | 0.8 | 1.0 | 4.6 |
| 50621 | SLC4A7 | | | 0.6 | 1.6 | 0.5 | 1.4 | 0.9 | 1.0 | 0.7 | 1.4 | 0.8 | 1.1 | 4.9 |
| 47960 | SLC7A5 | | | 1.7 | 1.6 | 1.1 | 1.1 | 0.9 | 1.0 | 0.6 | 1.4 | 1.0 | 1.0 | 4.5 |
| 49423 | SMPD1 | acid | sphingomyelin | 0.7 | 1.3 | 1.7 | 1.4 | 1.1 | 1.1 | 1.5 | 1.2 | 1.1 | 1.1 | 4.8 |
| | | sphingomylinase | phosphodiesterase | | | | | | | | | | | |
| | | | 1, acid | | | | | | | | | | | |
| | | | lysosomal | | | | | | | | | | | |
| 50548 | SMPD1 | acid | sphingomyelin | 0.8 | 1.0 | 1.5 | 1.4 | 1.1 | 1.1 | 1.4 | 1.3 | 1.1 | 1.0 | 4.8 |
| | | sphingomylinase | phosphodiesterase | | | | | | | | | | | |
| | | | 1, acid | | | | | | | | | | | |
| | | | lysosomal | | | | | | | | | | | |
| 47418 | SNK | | | 2.4 | 2.1 | 0.8 | 1.0 | 1.2 | 1.1 | [symbol] | 1.6 | 1.1 | 1.0 | 4.8 |
| 50162 | SP110 | IFI41, | interferon-induced | 5.3 | 3.4 | 1.5 | 1.4 | 1.2 | 1.1 | 1.1 | 1.1 | 1.1 | 1.0 | 4.6 |
| | | interferon | protein 41, 30 kD | | | | | | | | | | | |
| | | induced | | | | | | | | | | | | |
| | | protein 41 | | | | | | | | | | | | |
| 50706 | SP110 | IFI41, | interferon-induced | 5.4 | 3.2 | 1.5 | 1.4 | 1.2 | 1.1 | 1.1 | 1.1 | 1.1 | 1.0 | 4.6 |
| | | interferon | protein 41, 30 kD | | | | | | | | | | | |
| | | induced | | | | | | | | | | | | |
| | | protein 41 | | | | | | | | | | | | |
| 48711 | SPP1 | SPP1 | secreted | 0.5 | 1.7 | 0.4 | 2.0 | 1.1 | 1.0 | [symbol] | 1.6 | 1.2 | 1.0 | 5.7 |
| | | | phosphoprotein 1 | | | | | | | | | | | |
| | | | (osteopontin, bone | | | | | | | | | | | |
| | | | sialo | | | | | | | | | | | |
| 50397 | SPP1 | SPP1 | secreted | 0.5 | 1.6 | 0.4 | 2.3 | 1.1 | 1.0 | [symbol] | 1.5 | 1.1 | 1.0 | 5.7 |
| | | | phosphoprotein 1 | | | | | | | | | | | |
| | | | (osteopontin, bone | | | | | | | | | | | |
| | | | sialo | | | | | | | | | | | |
| 49446 | SRC | Src | v-src avian | 10.4 | 9.7 | 1.1 | 1.0 | 0.9 | 1.0 | [symbol] | 1.7 | 1.2 | 1.0 | 4.7 |
| | | | sarcoma | | | | | | | | | | | |
| | | | (Schmidt-Ruppin | | | | | | | | | | | |
| | | | A-2) viral onc | | | | | | | | | | | |
| 49990 | SRC | Src | v-src avian | 10.7 | 10.2 | 1.1 | 1.0 | 1.0 | 1.0 | [symbol] | 1.7 | 1.2 | 1.0 | 4.7 |
| | | | sarcoma | | | | | | | | | | | |
| | | | (Schmidt-Ruppin | | | | | | | | | | | |
| | | | A-2) viral onc | | | | | | | | | | | |
| 47195 | SRF | SRF | serum response | 0.7 | 1.1 | 0.7 | 1.3 | 1.0 | 1.0 | 0.7 | 1.4 | 1.1 | 1.0 | 4.6 |
| | | | factor (c-fos | | | | | | | | | | | |
| | | | serum response | | | | | | | | | | | |
| | | | elemen | | | | | | | | | | | |
| 47101 | SSA1 | | | 3.6 | 3.3 | 1.1 | 1.0 | 1.0 | 1.0 | 0.9 | 1.0 | 1.0 | 1.0 | 4.0 |
| 48009 | ST14 | ST14 | suppression of | 0.4 | 1.9 | 1.6 | 1.4 | 1.2 | 1.1 | [symbol] | 1.6 | 1.2 | 1.1 | 5.1 |
| | | | tumorigenicity 14 | | | | | | | | | | | |
| | | | (colon carcinoma, | | | | | | | | | | | |
| 50341 | ST14 | ST14 | suppression of | 0.4 | 1.8 | 1.6 | 1.5 | 1.2 | 1.1 | [symbol] | 1.6 | 1.1 | 1.0 | 5.1 |
| | | | tumorigenicity 14 | | | | | | | | | | | |
| | | | (colon carcinoma, | | | | | | | | | | | |
| 49746 | STAT1 | STAT1 | signal transducer | 4.7 | 4.3 | 1.9 | 1.6 | 1.2 | 1.0 | 1.2 | 1.1 | 1.1 | 1.0 | 4.7 |
| | | | and activator of | | | | | | | | | | | |
| | | | transcription 1 | | | | | | | | | | | |
| 50450 | STAT1 | STAT1 | signal transducer | 5.2 | 4.0 | 2.1 | 2.0 | 1.2 | 1.1 | 1.3 | 1.2 | 1.1 | 1.0 | 5.3 |
| | | | and activator of | | | | | | | | | | | |
| | | | transcription 1 | | | | | | | | | | | |
| 47727 | STAT2 | Stat2 | signal transducer | 4.3 | 4.1 | 1.1 | 1.1 | 0.9 | 1.1 | 0.9 | 1.0 | 1.1 | 1.0 | 4.1 |
| | | | and activator of | | | | | | | | | | | |
| | | | transcription 2 | | | | | | | | | | | |
| 47856 | STAT2 | Stat2 | signal transducer | 4.3 | 4.0 | 1.1 | 1.0 | 0.9 | 1.0 | 0.9 | 1.0 | 1.1 | 1.0 | 4.1 |
| | | | and activator of | | | | | | | | | | | |
| | | | transcription 2 | | | | | | | | | | | |
| 48987 | STAT5A | STAT5a | signal transducer | 3.4 | 2.7 | 1.1 | 1.0 | 1.1 | 1.0 | 1.2 | 1.2 | 1.1 | 1.1 | 4.3 |
| | | | and activator of | | | | | | | | | | | |
| | | | transcription 5 | | | | | | | | | | | |
| 50003 | STAT5A | STAT5a | signal transducer | 3.3 | 2.6 | 1.1 | 1.0 | 1.1 | 1.0 | 1.3 | 1.3 | 1.1 | 1.0 | 4.3 |
| | | | and activator of | | | | | | | | | | | |
| | | | transcription 5 | | | | | | | | | | | |
| 47634 | STATI2 | SOCS-2 | STAT induced | 3.1 | 2.6 | 1.0 | 1.0 | 1.0 | 1.0 | 0.7 | 1.2 | 1.1 | 1.0 | 4.2 |
| | | | STAT inhibitor-2 | | | | | | | | | | | |
| 50732 | STATI2 | SOCS-2 | STAT induced | 3.5 | 2.8 | 1.0 | 1.0 | 1.0 | 1.0 | 1.0 | 1.0 | 0.8 | 1.0 | 4.0 |
| | | | STAT inhibitor-2 | | | | | | | | | | | |
| 46796 | STC1 | | | 2.3 | 1.7 | 0.8 | 1.1 | 0.8 | 1.0 | [symbol] | 1.5 | [symbol] | 1.3 | 5.0 |
| 48769 | SUV39H1 | | | 0.8 | 1.2 | 0.6 | 1.4 | 1.0 | 1.0 | 1.0 | 1.0 | 1.0 | 1.0 | 4.4 |
| 48970 | SYK | Syk | spleen tyrosine | 0.6 | 1.3 | 1.5 | 1.4 | 1.1 | 1.0 | 1.4 | 1.3 | 1.0 | 1.0 | 4.7 |
| | | | kinase | | | | | | | | | | | |
| 49134 | SYK | Syk | spleen tyrosine | 0.6 | 1.5 | 1.5 | 1.4 | 1.1 | 1.0 | 1.4 | 1.2 | 0.9 | 1.0 | 4.6 |
| | | | kinase | | | | | | | | | | | |
| 47695 | TANK | I-TRAF, TANK | TRAF family | 2.5 | 2.4 | 1.0 | 1.0 | 0.9 | 1.0 | 1.3 | 1.0 | 1.0 | 1.0 | 4.0 |
| | | | member- | | | | | | | | | | | |
| | | | associated NFKB | | | | | | | | | | | |
| | | | activator | | | | | | | | | | | |
| 49372 | TAP1 | | | 6.9 | 6.0 | 1.4 | 1.2 | 1.1 | 1.0 | 1.0 | 1.0 | 1.1 | 1.0 | 4.2 |
| 48635 | TAX1BP1 | Tax1 BP, | Tax1 (human T- | 1.4 | 1.2 | 1.4 | 1.3 | 1.0 | 1.0 | 1.4 | 1.3 | 1.1 | 1.1 | 4.6 |
| | | T6BP, A20 | cell leukemia virus | | | | | | | | | | | |
| | | inhibitor | type I) binding | | | | | | | | | | | |
| 46907 | TBL1 | | | 1.2 | 1.0 | 1.7 | 1.5 | 1.1 | 1.0 | 1.3 | 1.0 | 1.0 | 1.0 | 4.5 |
| 49538 | TBXAS1 | | | 0.9 | 1.0 | 1.7 | 1.3 | 0.9 | 1.0 | [symbol] | 1.6 | 1.0 | 1.0 | 4.9 |
| 46613 | TCF12 | TCF12 | transcription factor | 2.0 | 1.8 | 1.3 | 1.3 | 1.0 | 1.0 | 1.1 | 1.1 | 1.0 | 1.0 | 4.4 |
| | | | 12 (HTF4, helix- | | | | | | | | | | | |
| | | | loop-helix tr | | | | | | | | | | | |
| 47324 | TCF7L2 | TCF7L2 | transcription factor | 1.3 | 1.1 | 0.6 | 1.4 | 0.9 | 1.0 | 0.8 | 1.1 | 0.9 | 1.1 | 4.6 |
| | | | 7-like 2 (T-cell | | | | | | | | | | | |
| | | | specific, HM | | | | | | | | | | | |
| 50172 | TCF8 | TCF8 | transcription factor | 0.5 | 1.8 | 0.6 | 1.3 | 1.0 | 1.0 | 0.7 | 1.3 | 0.9 | 1.1 | 4.6 |
| | | | 8 (represses | | | | | | | | | | | |
| | | | interleukin 2 ex | | | | | | | | | | | |
| 50139 | TCFL4 | TCFL4 | transcription | 0.6 | 1.6 | 0.9 | 1.1 | 1.0 | 1.0 | 0.7 | 1.3 | 0.9 | 1.0 | 4.4 |
| | | | factor-like 4 | | | | | | | | | | | |
| 50001 | TCFL4 | TCFL4 | transcription | 0.6 | 1.6 | 0.8 | 1.2 | 1.0 | 1.0 | 0.7 | 1.3 | 0.9 | 1.0 | 4.5 |
| | | | factor-like 4 | | | | | | | | | | | |
| 50370 | TCFL5 | TCFL5 | transcription | 5.6 | 2.5 | 0.8 | 1.0 | 1.0 | 1.0 | 1.1 | 1.0 | 1.0 | 1.0 | 4.0 |
| | | | factor-like 5 (basic | | | | | | | | | | | |
| | | | helix-loop-heli | | | | | | | | | | | |
| 49291 | TEAD4 | TEAD4 | TEA domain | 1.8 | 1.5 | 0.9 | 1.0 | 0.9 | 1.0 | 0.7 | 1.3 | 0.9 | 1.0 | 4.4 |
| | | | family member 4 | | | | | | | | | | | |
| 50587 | TEKT2 | | | 1.1 | 1.0 | 1.6 | 1.3 | 1.1 | 1.0 | 1.6 | 1.0 | 1.0 | 1.0 | 4.3 |
| 50529 | TFEC | TFEC | transcription factor | 2.5 | 2.3 | 1.4 | 1.4 | 1.1 | 1.0 | 1.5 | 1.4 | 1.0 | 1.0 | 4.8 |
| | | | EC | | | | | | | | | | | |
| 50658 | TFEC | TFEC | transcription factor | 2.5 | 2.2 | 1.5 | 1.4 | 1.1 | 1.0 | 1.5 | 1.3 | 1.0 | 1.0 | 4.7 |
| | | | EC | | | | | | | | | | | |
| 46991 | TFPI2 | TFPI2 | tissue factor | 4.2 | 3.9 | 0.1 | 5.1 | 0.7 | 1.2 | [symbol] | 4.2 | 0.9 | 1.0 | 11.5 |
| | | | pathway inhibitor 2 | | | | | | | | | | | |
| 48907 | TGFB3 | TGF, beta3 | transforming | 0.8 | 1.1 | 0.4 | 2.0 | 0.8 | 1.1 | 0.6 | 1.0 | 0.7 | 1.1 | 5.3 |
| | | | growth factor, beta 3 | | | | | | | | | | | |
| 47865 | TGFB3 | TGF, beta3 | transforming | 0.8 | 1.1 | 0.4 | 1.7 | 0.7 | 1.3 | [symbol] | 1.8 | 0.8 | 1.1 | 6.0 |
| | | | growth factor, beta 3 | | | | | | | | | | | |
| 48513 | TGFBR3 | | | 1.1 | 1.0 | 1.0 | 1.0 | 1.1 | 1.0 | [symbol] | 1.9 | 1.0 | 1.0 | 4.9 |
| 47593 | THBD | | | 0.5 | 1.6 | 0.6 | 1.8 | 0.8 | 1.1 | [symbol] | 3.5 | [symbol] | 1.4 | 7.8 |
| 47987 | THBS1 | THBS1 | thrombospondin 1 | 2.4 | 1.7 | 0.2 | 2.2 | 1.0 | 1.0 | [symbol] | 2.8 | 0.9 | 1.0 | 7.0 |
| 46857 | THBS1 | THBS1 | thrombospondin 1 | 5.0 | 2.4 | 0.6 | 1.5 | 0.8 | 1.0 | 0.8 | 1.2 | 0.9 | 1.0 | 4.7 |
| 47885 | THBS2 | THBS2 | thrombospondin 2 | 2.8 | 2.2 | 0.8 | 1.0 | 1.0 | 1.0 | 1.1 | 1.0 | 0.7 | 1.0 | 4.0 |
| 48627 | TIAM2 | TIAM2 | T-cell lymphoma | 2.9 | 2.5 | 1.0 | 1.0 | 1.0 | 1.0 | 1.0 | 1.0 | 0.9 | 1.0 | 4.0 |
| | | | invasion and | | | | | | | | | | | |
| | | | metastasis 2 | | | | | | | | | | | |
| 50035 | TIAM2 | TIAM2 | T-cell lymphoma | 2.5 | 2.1 | 1.0 | 1.0 | 1.0 | 1.0 | 0.9 | 1.0 | 0.9 | 1.0 | 4.0 |
| | | | invasion and | | | | | | | | | | | |
| | | | metastasis 2 | | | | | | | | | | | |
| 49420 | TIMP2 | TIMP2 | tissue inhibitor of | 0.8 | 1.1 | 2.2 | 2.0 | 1.2 | 1.2 | [symbol] | 1.8 | [symbol] | 1.3 | 6.3 |
| | | | metalloproteinase 2 | | | | | | | | | | | |
| 49785 | TIMP2 | TIMP2 | tissue inhibitor of | 0.8 | 1.1 | 2.0 | 1.9 | 1.2 | 1.2 | [symbol] | 1.7 | 1.3 | 1.2 | 5.9 |
| | | | metalloproteinase 2 | | | | | | | | | | | |
| 49818 | TIMP2 | TIMP2 | tissue inhibitor of | 0.8 | 1.1 | 1.9 | 1.8 | 1.2 | 1.2 | [symbol] | 1.6 | 1.3 | 1.1 | 5.7 |
| | | | metalloproteinase 2 | | | | | | | | | | | |
| 48754 | TIMP3 | | | 1.4 | 1.3 | 0.7 | 1.4 | 0.8 | 1.1 | 0.9 | 1.0 | 0.9 | 1.0 | 4.6 |
| 49298 | TIMP3 | | | 1.4 | 1.3 | 0.6 | 1.5 | 0.8 | 1.1 | 0.9 | 1.0 | 0.9 | 1.0 | 4.7 |
| 50294 | TM7SF2 | | | 0.6 | 1.3 | 1.3 | 1.1 | 0.7 | 1.2 | 1.7 | 1.3 | 0.7 | 1.1 | 4.8 |
| 49613 | TNF | TNFSF2, TNF, | tumor necrosis | 14.9 | 14.6 | 1.0 | 1.0 | 0.8 | 1.1 | [symbol] | 1.5 | 1.2 | 1.1 | 4.7 |
| | | alpha, tumor | factor (TNF | | | | | | | | | | | |
| | | necrosis | superfamily, | | | | | | | | | | | |
| | | factor, alpha | member 2) | | | | | | | | | | | |
| 47163 | TNF | TNFSF2, TNF, | tumor necrosis | 16.6 | 15.0 | 0.9 | 1.0 | 0.8 | 1.1 | 1.6 | 1.4 | 1.2 | 1.0 | 4.6 |
| | | alpha, tumor | factor (TNF | | | | | | | | | | | |
| | | necrosis | superfamily, | | | | | | | | | | | |
| | | factor, alpha | member 2) | | | | | | | | | | | |
| 47918 | TNFAIP1 | TNFAIP1 | tumor necrosis | 1.4 | 1.2 | 1.0 | 1.0 | 1.0 | 1.0 | 0.7 | 1.3 | 1.0 | 1.0 | 4.3 |
| | | | factor, alpha- | | | | | | | | | | | |
| | | | induced protein 1 | | | | | | | | | | | |
| | | | (en | | | | | | | | | | | |
| 47993 | TNFAIP2 | TNFAIP2 | tumor necrosis | 69.9 | 51.6 | 1.4 | 1.3 | 0.9 | 1.0 | 1.0 | 1.0 | 1.1 | 1.1 | 4.3 |
| | | | factor, alpha- | | | | | | | | | | | |
| | | | induced protein 2 | | | | | | | | | | | |
| 47642 | TNFAIP3 | A20, | tumor necrosis | 8.2 | 6.3 | 1.1 | 1.0 | 0.9 | 1.0 | 1.1 | 1.0 | 1.2 | 1.0 | 4.0 |
| | | TNFAIP2, Zn | factor, alpha- | | | | | | | | | | | |
| | | finger | induced protein 3 | | | | | | | | | | | |
| 47155 | TNFAIP6 | TNFAIP6 | tumor necrosis | 56.8 | 35.7 | 0.9 | 1.0 | 1.0 | 1.0 | 0.8 | 1.0 | 1.1 | 1.0 | 4.0 |
| | | | factor, alpha- | | | | | | | | | | | |
| | | | induced protein 6 | | | | | | | | | | | |
| 46803 | TNFAIP6 | TNFAIP6 | tumor necrosis | 62.1 | 44.9 | 0.7 | 1.0 | 1.0 | 1.0 | 0.8 | 1.0 | 1.1 | 1.0 | 4.0 |
| | | | factor, alpha- | | | | | | | | | | | |
| | | | induced protein 6 | | | | | | | | | | | |
| 47651 | TNFRSF21 | DR6 | death receptor 6 | 0.5 | 1.6 | 1.2 | 1.1 | 1.2 | 1.1 | 0.6 | 1.4 | 1.3 | 1.1 | 4.7 |
| 50458 | TNFRSF21 | DR6 | death receptor 6 | 0.5 | 1.7 | 1.2 | 1.0 | 1.2 | 1.2 | [symbol] | 1.5 | 1.2 | 1.0 | 4.6 |
| 47156 | TNFRSF5 | CD40 | tumor necrosis | 6.0 | 5.7 | 1.1 | 1.0 | 0.9 | 1.0 | 1.5 | 1.3 | 1.2 | 1.1 | 4.4 |
| | | | factor receptor | | | | | | | | | | | |
| | | | superfamily, | | | | | | | | | | | |
| | | | member | | | | | | | | | | | |
| 47027 | TNFRSF5 | CD40 | tumor necrosis | 5.7 | 5.5 | 1.1 | 1.0 | 1.0 | 1.0 | 1.4 | 1.3 | 1.1 | 1.0 | 4.3 |
| | | | factor receptor | | | | | | | | | | | |
| | | | superfamily, | | | | | | | | | | | |
| | | | member | | | | | | | | | | | |
| 48446 | TNFRSF6 | FAS | tumor necrosis | 5.4 | 5.0 | 1.2 | 1.0 | 1.0 | 1.0 | 1.2 | 1.0 | 1.2 | 1.1 | 4.1 |
| | | | factor receptor | | | | | | | | | | | |
| | | | superfamily, | | | | | | | | | | | |
| | | | member | | | | | | | | | | | |
| 50491 | TNFRSF6B | DcR3 | tumor necrosis | 0.7 | 1.3 | 0.6 | 1.2 | 0.9 | 1.1 | [symbol] | 1.6 | 0.9 | 1.0 | 4.9 |
| | | | factor receptor | | | | | | | | | | | |
| | | | superfamily, | | | | | | | | | | | |
| | | | member | | | | | | | | | | | |
| 47697 | TNFRSF6B | DcR3 | tumor necrosis | 0.8 | 1.2 | 0.5 | 1.7 | 0.9 | 1.0 | [symbol] | 1.9 | 0.9 | 1.0 | 5.6 |
| | | | factor receptor | | | | | | | | | | | |
| | | | superfamily, | | | | | | | | | | | |
| | | | member | | | | | | | | | | | |
| 50524 | TNFRSF6B | DcR3 | tumor necrosis | 0.7 | 1.2 | 0.7 | 1.1 | 0.9 | 1.0 | [symbol] | 1.5 | 0.9 | 1.0 | 4.6 |
| | | | factor receptor | | | | | | | | | | | |
| | | | superfamily, | | | | | | | | | | | |
| | | | member | | | | | | | | | | | |
| 49275 | TNFRSF9 | 4-1BB, CD137 | tumor necrosis | 4.0 | 2.2 | 1.1 | 1.0 | 1.0 | 1.0 | [symbol] | 1.5 | 1.4 | 1.2 | 4.7 |
| | | | factor receptor | | | | | | | | | | | |
| | | | superfamily, | | | | | | | | | | | |
| | | | member | | | | | | | | | | | |
| 47517 | TNFRSF9 | 4-1BB, CD137 | tumor necrosis | 3.8 | 2.1 | 1.1 | 1.0 | 0.9 | 1.0 | 1.4 | 1.3 | 1.5 | 1.1 | 4.4 |
| | | | factor receptor | | | | | | | | | | | |
| | | | superfamily, | | | | | | | | | | | |
| | | | member | | | | | | | | | | | |
| 47223 | TNFSF10 | TRAIL | tumor necrosis | 34.7 | 26.9 | 1.0 | 1.0 | 1.0 | 1.0 | 1.3 | 1.0 | 1.2 | 1.0 | 4.0 |
| | | | factor (ligand) | | | | | | | | | | | |
| | | | superfamily, | | | | | | | | | | | |
| | | | member | | | | | | | | | | | |
| 50533 | TNFSF9 | 4-1BBL | tumor necrosis | 3.0 | 2.3 | 1.0 | 1.0 | 1.0 | 1.0 | [symbol] | 1.8 | 1.3 | 1.2 | 4.9 |
| | | | factor (ligand) | | | | | | | | | | | |
| | | | superfamily, | | | | | | | | | | | |
| | | | member | | | | | | | | | | | |
| 50662 | TNFSF9 | 4-1BBL | tumor necrosis | 3.4 | 2.2 | 0.9 | 1.0 | 0.9 | 1.0 | [symbol] | 1.9 | 1.3 | 1.1 | 4.9 |
| | | | factor (ligand) | | | | | | | | | | | |
| | | | superfamily, | | | | | | | | | | | |
| | | | member | | | | | | | | | | | |
| 48111 | TP53BP1 | | | 0.8 | 1.3 | 0.8 | 1.1 | 1.0 | 1.0 | 0.7 | 1.3 | 0.9 | 1.1 | 4.5 |
| 48501 | TPST1 | | | 0.3 | 3.0 | 0.5 | 1.7 | 0.8 | 1.2 | 0.9 | 1.0 | 0.8 | 1.0 | 4.9 |
| 46819 | TRADD | TRADD | TNFRSF1A- | 2.5 | 2.3 | 1.2 | 1.0 | 1.1 | 1.0 | 1.1 | 1.0 | 1.2 | 1.1 | 4.1 |
| | | | associated via | | | | | | | | | | | |
| | | | death domain | | | | | | | | | | | |
| 48574 | TRAF1 | TRAF1 | TNF receptor- | 12.2 | 9.7 | 0.8 | 1.2 | 0.9 | 1.0 | 1.3 | 1.1 | 1.2 | 1.1 | 4.4 |
| | | | associated factor 1 | | | | | | | | | | | |
| 48445 | TRAF1 | TRAF1 | TNF receptor- | 11.8 | 9.0 | 0.7 | 1.2 | 0.9 | 1.0 | 1.3 | 1.1 | 1.2 | 1.1 | 4.4 |
| | | | associated factor 1 | | | | | | | | | | | |
| 50347 | TRAP1 | TRAP1, hsp90 | heat shock protein | 0.7 | 1.3 | 0.6 | 1.4 | 0.9 | 1.0 | [symbol] | 1.6 | 0.9 | 1.0 | 5.0 |
| | | related | 75 | | | | | | | | | | | |
| 47622 | TRAP1 | TRAP1, hsp90 | heat shock protein | 0.8 | 1.1 | 0.7 | 1.4 | 1.0 | 1.0 | 0.7 | 1.3 | 1.0 | 1.0 | 4.7 |
| | | related | 75 | | | | | | | | | | | |
| 47339 | TREM2 | | | 0.5 | 2.0 | 2.0 | 1.9 | 1.2 | 1.2 | [symbol] | 1.7 | 1.3 | 1.2 | 6.1 |
| 46941 | TUBA3 | | | 0.7 | 1.1 | 0.7 | 1.3 | 1.0 | 1.0 | 0.6 | 1.2 | 1.0 | 1.0 | 4.5 |
| 49402 | TWIST | | | 0.8 | 1.1 | 0.6 | 1.4 | 1.0 | 1.0 | [symbol] | 1.5 | 0.9 | 1.0 | 4.9 |
| 46574 | TXN | | | 1.5 | 1.3 | 1.2 | 1.1 | 1.0 | 1.0 | 1.6 | 1.4 | 1.2 | 1.1 | 4.5 |
| 49779 | TXNIP | VDUP1 | upregulated by | 0.6 | 1.4 | 1.5 | 1.2 | 1.2 | 1.1 | [symbol] | 1.6 | 1.1 | 1.1 | 5.0 |
| | | | 1,25- | | | | | | | | | | | |
| | | | dihydroxyvitamin | | | | | | | | | | | |
| | | | D-3 | | | | | | | | | | | |
| 49812 | TXNIP | VDUP1 | upregulated by | 0.6 | 1.2 | 1.5 | 1.2 | 1.2 | 1.2 | [symbol] | 1.7 | 1.1 | 1.0 | 5.1 |
| | | | 1,25- | | | | | | | | | | | |
| | | | dihydroxyvitamin | | | | | | | | | | | |
| | | | D-3 | | | | | | | | | | | |
| 47470 | TXNIP | VDUP1 | upregulated by | 0.8 | 1.0 | 1.4 | 1.1 | 1.1 | 1.1 | [symbol] | 1.6 | 1.1 | 1.0 | 4.8 |
| | | | 1,25- | | | | | | | | | | | |
| | | | dihydroxyvitamin | | | | | | | | | | | |
| | | | D-3 | | | | | | | | | | | |
| 49584 | TYRO3 | | | 4.3 | 3.7 | 1.1 | 1.0 | 1.0 | 1.0 | 0.9 | 1.0 | 0.9 | 1.0 | 4.0 |
| 48315 | U3-55K | | | 0.8 | 1.2 | 0.6 | 1.4 | 1.0 | 1.0 | 0.6 | 1.4 | 0.8 | 1.1 | 4.8 |
| 49522 | UCP2 | | | 0.6 | 1.5 | 1.8 | 1.7 | 1.3 | 1.2 | [symbol] | 1.6 | 1.2 | 1.1 | 5.5 |
| 49258 | UP | uridine | uridine | 1.4 | 1.2 | 0.5 | 1.6 | 0.9 | 1.1 | [symbol] | 1.5 | 0.8 | 1.2 | 5.4 |
| | | phosphorylase | phosphorylase | | | | | | | | | | | |
| 49129 | UP | uridine | uridine | 1.4 | 1.2 | 0.6 | 1.6 | 0.9 | 1.1 | [symbol] | 1.6 | 0.8 | 1.1 | 5.5 |
| | | phosphorylase | phosphorylase | | | | | | | | | | | |
| 46874 | VAV1 | Vav1 | vav 1 oncogene | 0.9 | 1.0 | 1.2 | 1.0 | 1.0 | 1.0 | 1.6 | 1.3 | 0.9 | 1.0 | 4.4 |
| 47298 | VAV3 | Vav3 | vav 3 oncogene | 0.5 | 2.1 | 0.7 | 1.3 | 1.1 | 1.0 | 0.6 | 1.2 | 0.9 | 1.0 | 4.6 |
| 48262 | VAV3 | Vav3 | vav 3 oncogene | 0.5 | 2.0 | 0.8 | 1.2 | 1.1 | 1.0 | [symbol] | 1.5 | 1.0 | 1.0 | 4.7 |
| 48185 | XAGE-1 | | | 1.0 | 1.0 | 1.4 | 1.3 | 1.0 | 1.0 | [symbol] | 1.7 | 1.2 | 1.1 | 5.1 |
| 50028 | XBP1 | XBP-1 | X-box binding | 3.0 | 2.7 | 1.2 | 1.1 | 1.0 | 1.0 | 1.4 | 1.3 | 1.3 | 1.2 | 4.5 |
| | | | protein 1 | | | | | | | | | | | |
| 46956 | YWHAQ | 14-3-3 theta, | tyrosine 3- | 1.1 | 1.0 | 1.0 | 1.0 | 1.0 | 1.0 | 1.6 | 1.4 | 1.1 | 1.0 | 4.5 |
| | | 14-3-3 tau, | monooxygenase/tryptophan | | | | | | | | | | | |
| | | HS1 | 5- | | | | | | | | | | | |
| | | | monooxygenas | | | | | | | | | | | |
| 50466 | ZFP36 | tristetrapro-lin | zinc finger protein | 2.0 | 1.0 | 1.6 | 1.5 | 1.1 | 1.0 | [symbol] | 1.5 | 1.1 | 1.0 | 4.9 |
| | | | homologous to | | | | | | | | | | | |
| | | | Zfp-36 in mouse | | | | | | | | | | | |
| 47836 | ZNF140 | | | 0.9 | 1.0 | 1.5 | 1.5 | 1.2 | 1.0 | 1.1 | 1.0 | 1.2 | 1.1 | 4.6 |

Drug Effects in THP1 Cells, Sum min fold change >=4.5
Genes in bold - ≧2-fold differential expression in response to LPS
Genes in italics - ≧1.5-fold differential expression in response to Clari and/or ≧1.3-fold differential expression in response to FMA alone
Genes in bold and italics - ≧1.5-fold differential expression in response to IL1 + Clari and/or ≧1.3-fold differential in response to IL1 + FMA

| | | | | | | | | | | | | | | |
|---|---|---|---|---|---|---|---|---|---|---|---|---|---|---|
| 48328 | FN1 | FN1 | fibronectin 1 | 0.9 | 1.1 | 0.0 | 10.1 | 0.7 | 1.4 | [symbol] | 12.4 | [symbol] | 1.3 | 25.2 |
| 48348 | FN1 | FN1 | fibronectin 1 | 0.8 | 1.1 | 0.0 | 9.6 | 0.6 | 1.4 | [symbol] | 7.6 | 0.8 | 1.2 | 19.9 |
| 46991 | TFPI2 | TFPI2 | tissue factor | 4.2 | 3.9 | 0.1 | 5.1 | 0.7 | 1.2 | [symbol] | 4.2 | 0.9 | 1.0 | 11.5 |
| | | | pathway inhibitor 2 | | | | | | | | | | | |
| 48334 | MMP7 | MMP7 | matrix | 1.5 | 1.4 | 0.1 | 4.3 | 0.9 | 1.1 | [symbol] | 4.4 | 0.9 | 1.0 | 10.8 |
| | | | metalloproteinase | | | | | | | | | | | |
| | | | 7 (matrilysin, | | | | | | | | | | | |
| | | | uterine) | | | | | | | | | | | |
| 46630 | MMP3 | MMP3 | matrix | 3.8 | 3.2 | 1.0 | 1.0 | 0.6 | 1.5 | [symbol] | 6.8 | 0.7 | 1.1 | 10.4 |
| | | | metalloproteinase | | | | | | | | | | | |
| | | | 3 (stromelysin 1, | | | | | | | | | | | |
| | | | progela | | | | | | | | | | | |
| 50403 | IGFBP3 | | | 2.8 | 2.2 | 0.2 | 4.2 | 0.9 | 1.0 | [symbol] | 3.8 | 0.7 | 1.1 | 10.1 |
| 48190 | SCYA20 | MIP-3a | small inducible | 8.1 | 5.6 | 0.1 | 6.1 | 0.7 | 1.4 | 0.6 | 1.0 | 1.1 | 1.0 | 9.5 |
| | | | cytokine subfamily | | | | | | | | | | | |
| | | | A (Cys—Cys), me | | | | | | | | | | | |
| 49786 | RGS16 | Rgs-16 | regulator of G- | 4.0 | 3.4 | 0.2 | 4.1 | 0.7 | 1.4 | [symbol] | 2.1 | 0.8 | 1.2 | 8.8 |
| | | | protein signalling | | | | | | | | | | | |
| | | | 16 | | | | | | | | | | | |
| 49586 | GRIN2C | | | 7.9 | 7.7 | 0.2 | 4.4 | 0.7 | 1.4 | 0.5 | 1.3 | 0.8 | 1.0 | 8.1 |
| 49060 | CTGF | CTGF | connective tissue | 108.1 | 106.7 | 1.0 | 1.0 | 1.3 | 1.0 | [symbol] | 4.9 | 1.1 | 1.0 | 7.9 |
| | | | growth factor | | | | | | | | | | | |
| 50715 | FCGR2A | CD32 | Fc fragment of | 1.7 | 1.7 | 3.8 | 3.1 | 1.1 | 1.1 | [symbol] | 2.5 | 1.3 | 1.2 | 7.8 |
| | | | IgG, low affinity | | | | | | | | | | | |
| | | | IIa, receptor for | | | | | | | | | | | |
| 47593 | THBD | | | 0.5 | 1.6 | 0.6 | 1.8 | 0.8 | 1.1 | [symbol] | 3.5 | [symbol] | 1.4 | 7.8 |
| 47895 | CRADD | RAIDD | CASP2 and | 1.5 | 1.4 | 0.4 | 2.2 | 0.8 | 1.1 | [symbol] | 3.3 | 0.8 | 1.2 | 7.7 |
| | | | RIPK1 domain | | | | | | | | | | | |
| | | | containing adaptor | | | | | | | | | | | |
| | | | with dea | | | | | | | | | | | |
| 46598 | SCYA20 | MIP-3a | small inducible | 8.6 | 7.8 | 0.2 | 4.0 | 0.7 | 1.4 | 0.6 | 1.3 | 1.0 | 1.0 | 7.7 |
| | | | cytokine subfamily | | | | | | | | | | | |
| | | | A (Cys—Cys), me | | | | | | | | | | | |
| 50125 | COL7A1 | COL7A1 | collagen, type VII, | 1.0 | 1.0 | 0.3 | 2.3 | 0.6 | 1.4 | [symbol] | 2.6 | [symbol] | 1.4 | 7.7 |
| | | | alpha 1 | | | | | | | | | | | |
| | | | (epidermolysis | | | | | | | | | | | |
| | | | bullosa | | | | | | | | | | | |
| 46876 | GNG11 | G protein, | guanine | 1.2 | 1.0 | 0.4 | 2.6 | 0.8 | 1.1 | [symbol] | 2.8 | 0.9 | 1.0 | 7.5 |
| | | gamma 11 | nucleotide binding | | | | | | | | | | | |
| | | | protein 11 | | | | | | | | | | | |
| 46856 | IL8 | interleukin-8 | interleukin 8 | 7.2 | 6.7 | 0.2 | 3.8 | 0.6 | 1.4 | 1.2 | 1.2 | 1.2 | 1.0 | 7.4 |
| 49995 | FCGR2A | CD32 | Fc fragment of | 2.4 | 2.0 | 3.6 | 2.7 | 1.1 | 1.0 | [symbol] | 2.5 | 1.2 | 1.2 | 7.4 |
| | | | IgG, low affinity | | | | | | | | | | | |
| | | | IIa, receptor for | | | | | | | | | | | |
| 50705 | COL7A1 | COL7A1 | collagen, type VII, | 1.0 | 1.0 | 0.3 | 2.0 | 0.6 | 1.4 | [symbol] | 2.6 | [symbol] | 1.4 | 7.4 |
| | | | alpha 1 | | | | | | | | | | | |
| | | | (epidermolysis | | | | | | | | | | | |
| | | | bullosa | | | | | | | | | | | |
| 46970 | IL1B | IL-1, beta | interleukin 1, beta | 3.3 | 2.3 | 0.2 | 3.9 | 0.7 | 1.2 | 1.2 | 1.2 | 1.2 | 1.1 | 7.3 |
| 49665 | CRADD | RAIDD | CASP2 and | 1.6 | 1.2 | 0.4 | 1.9 | 0.8 | 1.1 | [symbol] | 3.2 | 0.7 | 1.0 | 7.3 |
| | | | RIPK1 domain | | | | | | | | | | | |
| | | | containing adaptor | | | | | | | | | | | |
| | | | with dea | | | | | | | | | | | |
| 48966 | MMP10 | MMP10 | matrix | 1.7 | 1.6 | 0.5 | 1.8 | 0.6 | 1.6 | [symbol] | 2.3 | [symbol] | 1.5 | 7.2 |
| | | | metalloproteinase | | | | | | | | | | | |
| | | | 10 (stromelysin 2) | | | | | | | | | | | |
| 48273 | FCGR2A | CD32 | Fc fragment of | 2.0 | 1.9 | 3.8 | 2.5 | 1.0 | 1.0 | [symbol] | 2.3 | [symbol] | 1.3 | 7.0 |
| | | | IgG, low affinity | | | | | | | | | | | |
| | | | IIa, receptor for | | | | | | | | | | | |
| 47987 | THBS1 | THBS1 | thrombospondin 1 | 2.4 | 1.7 | 0.2 | 2.2 | 1.0 | 1.0 | [symbol] | 2.8 | 0.9 | 1.0 | 7.0 |
| 48977 | FCGR2A | CD32 | Fc fragment of | 2.1 | 1.9 | 3.0 | 2.1 | 1.0 | 1.0 | [symbol] | 2.5 | 1.2 | 1.2 | 6.9 |
| | | | IgG, low affinity | | | | | | | | | | | |
| | | | IIa, receptor for | | | | | | | | | | | |
| 47656 | IL1B | IL-1, beta | interleukin 1, beta | 2.5 | 2.4 | 0.3 | 3.5 | 0.7 | 1.1 | 1.3 | 1.1 | 1.2 | 1.1 | 6.8 |
| 46949 | AREG | amphiregulin | amphiregulin | 1.9 | 1.4 | 0.8 | 1.0 | 0.5 | 1.8 | [symbol] | 2.4 | [symbol] | 1.4 | 6.6 |
| | | | (schwannoma- | | | | | | | | | | | |
| | | | derived growth | | | | | | | | | | | |
| | | | factor) | | | | | | | | | | | |
| 46982 | AREG | amphiregulin | amphiregulin | 1.8 | 1.6 | 0.8 | 1.1 | 0.5 | 1.8 | [symbol] | 2.2 | [symbol] | 1.5 | 6.5 |
| | | | (schwannoma- | | | | | | | | | | | |
| | | | derived growth | | | | | | | | | | | |
| | | | factor) | | | | | | | | | | | |
| 46986 | AREG | amphiregulin | amphiregulin | 1.8 | 1.5 | 0.8 | 1.0 | 0.5 | 1.8 | [symbol] | 2.0 | [symbol] | 1.6 | 6.5 |
| | | | (schwannoma- | | | | | | | | | | | |
| | | | derived growth | | | | | | | | | | | |
| | | | factor) | | | | | | | | | | | |
| 46953 | AREG | amphiregulin | amphiregulin | 1.8 | 1.5 | 0.8 | 1.0 | 0.5 | 1.9 | [symbol] | 2.0 | [symbol] | 1.5 | 6.4 |
| | | | (schwannoma- | | | | | | | | | | | |
| | | | derived growth | | | | | | | | | | | |
| | | | factor) | | | | | | | | | | | |
| 48784 | RGS16 | Rgs-16 | regulator of G- | 3.5 | 3.2 | 0.4 | 2.0 | 0.7 | 1.3 | [symbol] | 2.1 | 0.9 | 1.0 | 6.4 |
| | | | protein signalling | | | | | | | | | | | |
| | | | 16 | | | | | | | | | | | |
| 47537 | PLAU | urokinase-type | plasminogen | 1.9 | 1.7 | 0.6 | 1.3 | 0.8 | 1.3 | [symbol] | 2.6 | 0.8 | 1.2 | 6.3 |
| | | plasminogen | activator, | | | | | | | | | | | |
| | | activator | urokinase | | | | | | | | | | | |
| 49420 | TIMP2 | TIMP2 | tissue inhibitor of | 0.8 | 1.1 | 2.2 | 2.0 | 1.2 | 1.2 | [symbol] | 1.8 | [symbol] | 1.3 | 6.3 |
| | | | metalloproteinase 2 | | | | | | | | | | | |
| 50120 | S100A4 | S100A4 | S100 calcium- | 1.0 | 1.0 | 2.2 | 2.0 | 1.2 | 1.1 | [symbol] | 1.9 | 1.2 | 1.1 | 6.2 |
| | | | binding protein A4 | | | | | | | | | | | |
| | | | (calcium protein, | | | | | | | | | | | |
| 49424 | PLAU | urokinase-type | plasminogen | 1.8 | 1.5 | 0.7 | 1.1 | 0.7 | 1.3 | [symbol] | 2.5 | 0.8 | 1.2 | 6.1 |
| | | plasminogen | activator, | | | | | | | | | | | |
| | | activator | urokinase | | | | | | | | | | | |
| 49758 | IL10RA | IL-10R alpha | interleukin 10 | 9.5 | 7.9 | 2.0 | 1.8 | 1.2 | 1.0 | [symbol] | 2.1 | 1.4 | 1.2 | 6.1 |
| | | | receptor, alpha | | | | | | | | | | | |
| 47339 | TREM2 | | | 0.5 | 2.0 | 2.0 | 1.9 | 1.2 | 1.2 | [symbol] | 1.7 | 1.3 | 1.2 | 6.1 |
| 50345 | AGT | | serine (or | 0.7 | 1.1 | 2.1 | 1.7 | 1.1 | 1.1 | [symbol] | 2.2 | 1.1 | 1.0 | 6.0 |
| | | | cysteine) | | | | | | | | | | | |
| | | | proteinase | | | | | | | | | | | |
| | | | inhibitor, clade A | | | | | | | | | | | |
| 47893 | SCYA4 | MIP-1beta | small inducible | 48.7 | 24.7 | 0.3 | 2.8 | 0.8 | 1.0 | 1.2 | 1.2 | 1.2 | 1.1 | 6.0 |
| | | | cytokine A4 | | | | | | | | | | | |
| | | | (homologous to | | | | | | | | | | | |
| | | | mouse M | | | | | | | | | | | |
| 49326 | FCGR2A | CD32 | Fc fragment of | 2.5 | 2.1 | 3.2 | 2.1 | 1.0 | 1.0 | [symbol] | 1.9 | 1.2 | 1.1 | 6.0 |
| | | | IgG, low affinity | | | | | | | | | | | |
| | | | IIa, receptor for | | | | | | | | | | | |
| 47865 | TGFB3 | TGF, beta3 | transforming | 0.8 | 1.1 | 0.4 | 1.7 | 0.7 | 1.3 | [symbol] | 1.8 | 0.8 | 1.1 | 6.0 |
| | | | growth factor, beta 3 | | | | | | | | | | | |
| 49305 | HLA-DRB1 | MHCII, DR | major | 0.9 | 1.0 | 2.1 | 1.8 | 1.1 | 1.1 | [symbol] | 1.8 | 1.2 | 1.2 | 6.0 |
| | | beta 1 | histocompatibility | | | | | | | | | | | |
| | | | complex, class II, | | | | | | | | | | | |
| | | | DR bet | | | | | | | | | | | |
| 49785 | TIMP2 | TIMP2 | tissue inhibitor of | 0.8 | 1.1 | 2.0 | 1.9 | 1.2 | 1.2 | [symbol] | 1.7 | 1.3 | 1.2 | 5.9 |
| | | | metalloproteinase 2 | | | | | | | | | | | |
| 47632 | GRO2 | GRO2 | GRO2 oncogene | 25.4 | 16.7 | 0.4 | 2.5 | 0.7 | 1.4 | 0.7 | 1.0 | 0.9 | 1.0 | 5.9 |
| 50387 | PPIC | cyclophilin C | peptidylprolyl | 0.9 | 1.0 | 2.0 | 1.9 | 1.2 | 1.1 | [symbol] | 1.9 | 1.2 | 1.0 | 5.9 |
| | | | isomerase C | | | | | | | | | | | |
| | | | (cyclophilin C) | | | | | | | | | | | |
| 50669 | ECM1 | ECM1 | extracellular | 0.9 | 1.0 | 2.0 | 1.8 | 1.1 | 1.0 | [symbol] | 1.9 | 1.2 | 1.1 | 5.8 |
| | | | matrix protein 1 | | | | | | | | | | | |
| 47179 | HLA-DRB1 | MHCII, DR | major | 0.9 | 1.0 | 2.0 | 1.7 | 1.2 | 1.1 | [symbol] | 1.8 | 1.3 | 1.2 | 5.8 |
| | | beta 1 | histocompatibility | | | | | | | | | | | |
| | | | complex, class II, | | | | | | | | | | | |
| | | | DR bet | | | | | | | | | | | |
| 47040 | IL10RA | IL-10R alpha | interleukin 10 | 11.8 | 10.6 | 2.1 | 1.7 | 1.1 | 1.0 | [symbol] | 1.8 | 1.5 | 1.2 | 5.8 |
| | | | receptor, alpha | | | | | | | | | | | |
| 50026 | SCYA2 | MCP1 | small inducible | 24.1 | 13.0 | 0.4 | 2.4 | 1.2 | 1.0 | 0.6 | 1.3 | 1.1 | 1.0 | 5.8 |
| | | | cytokine A2 | | | | | | | | | | | |
| | | | (monocyte | | | | | | | | | | | |
| | | | chemotactic | | | | | | | | | | | |
| 48227 | NRG1 | neuregulin1, | neuregulin 1 | 5.8 | 3.5 | 0.4 | 1.9 | 0.7 | 1.2 | [symbol] | 1.5 | 0.8 | 1.1 | 5.8 |
| | | neu diff. | | | | | | | | | | | | |
| | | Factor, | | | | | | | | | | | | |
| | | heregulin | | | | | | | | | | | | |
| 49293 | HLA-DRB3 | MHCII, DR | major | 0.9 | 1.0 | 2.0 | 1.8 | 1.1 | 1.1 | [symbol] | 1.7 | 1.1 | 1.1 | 5.8 |
| | | beta 3 | histocompatibility | | | | | | | | | | | |
| | | | complex, class II, | | | | | | | | | | | |
| | | | DR bet | | | | | | | | | | | |
| 49818 | TIMP2 | TIMP2 | tissue inhibitor of | 0.8 | 1.1 | 1.9 | 1.8 | 1.2 | 1.2 | [symbol] | 1.6 | 1.3 | 1.1 | 5.7 |
| | | | metalloproteinase 2 | | | | | | | | | | | |
| 50397 | SPP1 | SPP1 | secreted | 0.5 | 1.6 | 0.4 | 2.3 | 1.1 | 1.0 | [symbol] | 1.5 | 1.1 | 1.0 | 5.7 |
| | | | phosphoprotein 1 | | | | | | | | | | | |
| | | | (osteopontin, bone | | | | | | | | | | | |
| | | | sialo | | | | | | | | | | | |
| 48711 | SPP1 | SPP1 | secreted | 0.5 | 1.7 | 0.4 | 2.0 | 1.1 | 1.0 | [symbol] | 1.6 | 1.2 | 1.0 | 5.7 |
| | | | phosphoprotein 1 | | | | | | | | | | | |
| | | | (osteopontin, bone | | | | | | | | | | | |
| | | | sialo | | | | | | | | | | | |
| 48911 | ECM1 | ECM1 | extracellular | 0.9 | 1.0 | 1.9 | 1.7 | 1.1 | 1.0 | [symbol] | 1.8 | 1.2 | 1.1 | 5.7 |
| | | | matrix protein 1 | | | | | | | | | | | |
| 48827 | PBX3 | | | 0.9 | 1.0 | 2.1 | 1.8 | 1.1 | 1.0 | [symbol] | 1.8 | 1.1 | 1.0 | 5.7 |
| 48589 | HLA-DRB1 | MHCII, DR | major | 1.0 | 1.0 | 2.0 | 1.8 | 1.1 | 1.1 | [symbol] | 1.7 | 1.2 | 1.2 | 5.7 |
| | | beta 1 | histocompatibility | | | | | | | | | | | |
| | | | complex, class II, | | | | | | | | | | | |
| | | | DR bet | | | | | | | | | | | |
| 49838 | | | | 1.0 | 1.0 | 1.9 | 1.7 | 1.1 | 1.0 | [symbol] | 1.9 | 1.1 | 1.0 | 5.7 |
| 47887 | HLA-DRB1 | MHCII, DR | major | 1.1 | 1.0 | 1.8 | 1.7 | 1.1 | 1.0 | [symbol] | 1.7 | 1.2 | 1.2 | 5.6 |
| | | beta 1 | histocompatibility | | | | | | | | | | | |
| | | | complex, class II, | | | | | | | | | | | |
| | | | DR bet | | | | | | | | | | | |
| 47539 | F2 | F2 | coagulation factor | 1.7 | 1.5 | 0.7 | 1.2 | 0.8 | 1.1 | [symbol] | 2.1 | 0.8 | 1.2 | 5.6 |
| | | | II (thrombin) | | | | | | | | | | | |
| 49124 | MMP7 | MMP7 | matrix | 1.4 | 1.3 | 0.4 | 1.5 | 0.9 | 1.0 | [symbol] | 2.1 | 0.9 | 1.0 | 5.6 |
| | | | metalloproteinase | | | | | | | | | | | |
| | | | 7 (matrilysin, | | | | | | | | | | | |
| | | | uterine) | | | | | | | | | | | |
| 49681 | HSXIAPAF1 | XIAP assoc | XIAP associated | 9.5 | 9.3 | 2.8 | 2.6 | 1.1 | 1.0 | 1.0 | 1.0 | 0.9 | 1.0 | 5.6 |
| | | factor 1 | factor-1 | | | | | | | | | | | |
| 47697 | TNFRSF6B | DcR3 | tumor necrosis | 0.8 | 1.2 | 0.5 | 1.7 | 0.9 | 1.0 | [symbol] | 1.9 | 0.9 | 1.0 | 5.6 |
| | | | factor receptor | | | | | | | | | | | |
| | | | superfamily, | | | | | | | | | | | |
| | | | member | | | | | | | | | | | |
| 46926 | HLA-DRB1 | MHCII, DR | major | 1.0 | 1.0 | 2.0 | 1.5 | 1.2 | 1.1 | [symbol] | 1.7 | 1.3 | 1.2 | 5.6 |
| | | beta 1 | histocompatibility | | | | | | | | | | | |
| | | | complex, class II, | | | | | | | | | | | |
| | | | DR bet | | | | | | | | | | | |
| 49277 | GNG11 | G protein, | guanine | 1.1 | 1.0 | 0.5 | 1.6 | 0.8 | 1.1 | [symbol] | 1.9 | 1.0 | 1.0 | 5.6 |
| | | gamma 11 | nucleotide binding | | | | | | | | | | | |
| | | | protein 11 | | | | | | | | | | | |
| 47318 | S100A4 | S100A4 | S100 calcium- | 1.0 | 1.0 | 2.0 | 1.8 | 1.2 | 1.0 | [symbol] | 1.6 | 1.2 | 1.1 | 5.6 |
| | | | binding protein A4 | | | | | | | | | | | |
| | | | (calcium protein, | | | | | | | | | | | |
| 46983 | PPIC | cyclophilin C | peptidylprolyl | 1.0 | 1.0 | 2.0 | 1.5 | 1.2 | 1.2 | [symbol] | 1.6 | 1.4 | 1.2 | 5.6 |
| | | | isomerase C | | | | | | | | | | | |
| | | | (cyclophilin C) | | | | | | | | | | | |
| 50665 | PTGS2 | PTGS2, COX2 | prostaglandin- | 32.8 | 31.2 | 1.0 | 1.0 | 0.8 | 1.2 | [symbol] | 2.3 | 1.3 | 1.1 | 5.6 |
| | | | endoperoxide | | | | | | | | | | | |
| | | | synthase 2 | | | | | | | | | | | |
| | | | (prostagland | | | | | | | | | | | |
| 48605 | HSXIAPAF1 | XIAP assoc | XIAP associated | 13.2 | 8.9 | 2.7 | 2.5 | 1.2 | 1.0 | 1.1 | 1.0 | 1.0 | 1.0 | 5.6 |
| | | factor 1 | factor-1 | | | | | | | | | | | |
| 48074 | | | | 1.4 | 1.1 | 1.8 | 1.6 | 1.1 | 1.1 | [symbol] | 1.8 | 1.1 | 1.1 | 5.6 |
| 49692 | GRO3 | GRO3 | GRO3 oncogene | 33.5 | 17.1 | 0.4 | 2.2 | 0.7 | 1.4 | 0.7 | 1.0 | 1.0 | 1.0 | 5.6 |
| 48330 | GRO3 | GRO3 | GRO3 oncogene | 36.2 | 17.9 | 0.4 | 2.2 | 0.7 | 1.2 | 0.8 | 1.0 | 0.9 | 1.1 | 5.5 |
| 49754 | GRO1 | GRO1 | GRO1 oncogene | 44.5 | 27.1 | 0.4 | 2.2 | 0.7 | 1.3 | 0.8 | 1.0 | 1.0 | 1.0 | 5.5 |
| | | | (melanoma growth | | | | | | | | | | | |
| | | | stimulating activit | | | | | | | | | | | |
| 47513 | PRKCB1 | PKC, beta | protein kinase C, | 1.0 | 1.0 | 2.0 | 1.8 | 1.1 | 1.0 | [symbol] | 1.7 | 1.2 | 1.1 | 5.5 |
| | | | beta 1 | | | | | | | | | | | |
| 48839 | FCGR1A | | | 0.8 | 1.1 | 2.2 | 2.1 | 1.2 | 1.2 | 1.4 | 1.3 | 1.1 | 1.0 | 5.5 |
| 49129 | UP | uridine | uridine | 1.4 | 1.2 | 0.6 | 1.6 | 0.9 | 1.1 | [symbol] | 1.6 | 0.8 | 1.1 | 5.5 |
| | | phosphorylase | phosphorylase | | | | | | | | | | | |
| 49669 | GALNAC4S-6ST | BRAG | B cell RAG | 0.5 | 1.5 | 0.5 | 1.7 | 1.0 | 1.0 | [symbol] | 1.8 | 0.7 | 1.0 | 5.5 |
| | | | associated protein | | | | | | | | | | | |
| 46787 | SERPINI1 | | serine (or | 1.1 | 1.0 | 2.6 | 1.8 | 1.1 | 1.1 | [symbol] | 1.6 | 1.1 | 1.1 | 5.5 |
| | | | cysteine) | | | | | | | | | | | |
| | | | proteinase | | | | | | | | | | | |
| | | | inhibitor, clade I | | | | | | | | | | | |
| 49052 | CXCR4 | CXCR4 | chemokine (C—X—C | 2.0 | 1.7 | 0.4 | 2.2 | 0.9 | 1.0 | 0.7 | 1.3 | 1.1 | 1.0 | 5.5 |
| | | | motif), receptor 4 | | | | | | | | | | | |
| | | | (fusin) | | | | | | | | | | | |
| 48271 | ADCY7 | adenylate | adenylate cyclase 7 | 1.1 | 1.0 | 1.8 | 1.3 | 1.2 | 1.2 | [symbol] | 1.8 | 1.3 | 1.2 | 5.5 |
| | | cyclase | | | | | | | | | | | | |
| 49522 | UCP2 | | | 0.6 | 1.5 | 1.8 | 1.7 | 1.3 | 1.2 | [symbol] | 1.6 | 1.2 | 1.1 | 5.5 |
| 50385 | HLA-DRB1 | MHCII, DR | major | 0.9 | 1.1 | 1.9 | 1.7 | 1.1 | 1.0 | [symbol] | 1.8 | 1.2 | 1.0 | 5.5 |
| | | beta 1 | histocompatibility | | | | | | | | | | | |
| | | | complex, class II, | | | | | | | | | | | |
| | | | DR bet | | | | | | | | | | | |
| 46877 | DTR | HB-EGF, | diphtheria toxin | 2.4 | 2.0 | 0.5 | 1.4 | 0.7 | 1.1 | [symbol] | 1.6 | [symbol] | 1.4 | 5.5 |
| | | diptheria toxin | receptor (heparin- | | | | | | | | | | | |
| | | receptor | binding epiderm | | | | | | | | | | | |
| 49208 | | | | 0.5 | 1.7 | 0.6 | 1.4 | 0.8 | 1.1 | [symbol] | 1.8 | 0.8 | 1.2 | 5.5 |
| 49287 | ADCY7 | adenylate | adenylate cyclase 7 | 1.1 | 1.0 | 1.9 | 1.6 | 1.2 | 1.1 | [symbol] | 1.7 | 1.2 | 1.1 | 5.5 |
| | | cyclase | | | | | | | | | | | | |
| 50380 | | | | 0.9 | 1.0 | 1.9 | 1.7 | 1.2 | 1.0 | [symbol] | 1.8 | 1.1 | 1.0 | 5.5 |
| 50383 | DTR | HB-EGF, | diphtheria toxin | 1.7 | 1.3 | 0.6 | 1.5 | 0.7 | 1.2 | [symbol] | 1.5 | [symbol] | 1.3 | 5.5 |
| | | diptheria toxin | receptor (heparin- | | | | | | | | | | | |
| | | receptor | binding epiderm | | | | | | | | | | | |
| 47030 | | | | 1.3 | 1.2 | 1.9 | 1.7 | 1.1 | 1.1 | 1.5 | 1.3 | [symbol] | 1.4 | 5.4 |
| 50597 | PDE7A | | | 0.9 | 1.0 | 1.8 | 1.7 | 1.0 | 1.0 | [symbol] | 1.7 | 0.9 | 1.0 | 5.4 |
| 47217 | ADAMTS1 | ADAMTS1 | a disintegrin-like | 1.9 | 1.6 | 2.3 | 2.0 | 1.1 | 1.0 | 1.5 | 1.4 | 1.1 | 1.0 | 5.4 |
| | | | and | | | | | | | | | | | |
| | | | metalloprotease | | | | | | | | | | | |
| | | | (reprolysin | | | | | | | | | | | |
| 49217 | HMGIC | | | 0.6 | 1.1 | 0.7 | 1.3 | 0.9 | 1.0 | [symbol] | 1.8 | [symbol] | 1.3 | 5.4 |
| 50361 | HMGIY | HMG-I/Y | high-mobility | 0.7 | 1.3 | 0.5 | 1.7 | 0.9 | 1.1 | [symbol] | 1.5 | 0.8 | 1.1 | 5.4 |
| | | | group (nonhistone | | | | | | | | | | | |
| | | | chromosomal) | | | | | | | | | | | |
| | | | prote | | | | | | | | | | | |
| 49258 | UP | uridine | uridine | 1.4 | 1.2 | 0.5 | 1.6 | 0.9 | 1.1 | [symbol] | 1.5 | 0.8 | 1.2 | 5.4 |
| | | phosphorylase | phosphorylase | | | | | | | | | | | |
| 49347 | HNRPA3 | | | 0.9 | 1.1 | 0.5 | 1.7 | 1.0 | 1.0 | [symbol] | 1.7 | 1.0 | 1.0 | 5.4 |
| 47704 | PLOD | PLOD | procollagen- | 0.6 | 1.5 | 1.9 | 1.7 | 0.9 | 1.0 | [symbol] | 1.7 | 1.0 | 1.0 | 5.4 |
| | | | lysine, 2- | | | | | | | | | | | |
| | | | oxoglutarate 5- | | | | | | | | | | | |
| | | | dioxygenase ( | | | | | | | | | | | |
| 47857 | COL9A2 | COL9A2 | collagen, type IX, | 1.0 | 1.0 | 1.9 | 1.7 | 1.2 | 1.1 | [symbol] | 1.5 | 1.1 | 1.1 | 5.4 |
| | | | alpha 2 | | | | | | | | | | | |
| 46862 | PPIF | cyclophilin F | peptidylprolyl | 1.3 | 1.2 | 0.8 | 1.0 | 0.9 | 1.1 | [symbol] | 1.9 | [symbol] | 1.3 | 5.4 |
| | | | isomerase F | | | | | | | | | | | |
| | | | (cyclophilin F) | | | | | | | | | | | |
| 50150 | MAP3K4 | MEKK4 | mitogen-activated | 0.9 | 1.0 | 0.7 | 1.2 | 0.9 | 1.0 | [symbol] | 2.1 | 0.8 | 1.0 | 5.4 |
| | | | protein kinase | | | | | | | | | | | |
| | | | kinase kinase 4 | | | | | | | | | | | |
| 49106 | PLOD | PLOD | procollagen- | 0.6 | 1.7 | 1.9 | 1.7 | 1.0 | 1.0 | [symbol] | 1.6 | 0.9 | 1.0 | 5.3 |
| | | | lysine, 2- | | | | | | | | | | | |
| | | | oxoglutarate 5- | | | | | | | | | | | |
| | | | dioxygenase ( | | | | | | | | | | | |
| 48582 | MMP13 | MMP13 | matrix | 0.9 | 1.1 | 1.6 | 1.5 | 1.1 | 1.1 | [symbol] | 1.7 | 1.2 | 1.0 | 5.3 |
| | | | metalloproteinase | | | | | | | | | | | |
| | | | 13 (collagenase 3) | | | | | | | | | | | |
| 50462 | GRO1 | GRO1 | GRO1 oncogene | 33.8 | 21.0 | 0.4 | 2.1 | 0.8 | 1.2 | 0.7 | 1.0 | 1.0 | 1.0 | 5.3 |
| | | | (melanoma growth | | | | | | | | | | | |
| | | | stimulating activit | | | | | | | | | | | |
| 49162 | DKFZP761N09121 | | | 1.0 | 1.0 | 1.9 | 1.6 | 1.1 | 1.0 | [symbol] | 1.7 | 1.1 | 1.0 | 5.3 |
| 48281 | SERPINE2 | | serine (or | 34.6 | 17.3 | 0.4 | 2.2 | 1.0 | 1.0 | 0.6 | 1.1 | 1.1 | 1.0 | 5.3 |
| | | | cysteine) | | | | | | | | | | | |
| | | | proteinase | | | | | | | | | | | |
| | | | inhibitor, clade E | | | | | | | | | | | |
| 46988 | EREG | epiregulin | epiregulin | 4.5 | 3.9 | 1.0 | 1.0 | 1.0 | 1.0 | [symbol] | 2.2 | 0.8 | 1.1 | 5.3 |
| 48400 | SERPINE2 | | serine (or | 26.7 | 13.2 | 0.4 | 2.2 | 1.0 | 1.0 | 0.7 | 1.1 | 1.0 | 1.0 | 5.3 |
| | | | cysteine) | | | | | | | | | | | |
| | | | proteinase | | | | | | | | | | | |
| | | | inhibitor, clade E | | | | | | | | | | | |
| 49073 | PLOD | PLOD | procollagen- | 0.7 | 1.4 | 1.9 | 1.7 | 1.0 | 1.0 | [symbol] | 1.6 | 1.0 | 1.0 | 5.3 |
| | | | lysine, 2- | | | | | | | | | | | |
| | | | oxoglutarate 5- | | | | | | | | | | | |
| | | | dioxygenase ( | | | | | | | | | | | |
| 48907 | TGFB3 | TGF, beta3 | transforming | 0.8 | 1.1 | 0.4 | 2.0 | 0.8 | 1.1 | 0.6 | 1.0 | 0.7 | 1.1 | 5.3 |
| | | | growth factor, beta 3 | | | | | | | | | | | |
| 47671 | PLOD | PLOD | procollagen- | 0.6 | 1.5 | 1.9 | 1.7 | 0.9 | 1.0 | [symbol] | 1.6 | 1.1 | 1.0 | 5.3 |
| | | | lysine, 2- | | | | | | | | | | | |
| | | | oxoglutarate 5- | | | | | | | | | | | |
| | | | dioxygenase ( | | | | | | | | | | | |
| 48530 | APLP2 | | | 0.9 | 1.1 | 1.7 | 1.7 | 1.0 | 1.0 | [symbol] | 1.6 | 1.0 | 1.0 | 5.3 |
| 48267 | CASP1 | caspase 1, | caspase 1, | 5.9 | 4.1 | 2.2 | 1.9 | 1.2 | 1.0 | 1.3 | 1.1 | 1.3 | 1.2 | 5.3 |
| | | ICE | apoptosis-related | | | | | | | | | | | |
| | | | cysteine protease | | | | | | | | | | | |
| | | | (in | | | | | | | | | | | |
| 50450 | STAT1 | STAT1 | signal transducer | 5.2 | 4.0 | 2.1 | 2.0 | 1.2 | 1.1 | 1.3 | 1.2 | 1.1 | 1.0 | 5.3 |
| | | | and activator of | | | | | | | | | | | |
| | | | transcription 1 | | | | | | | | | | | |
| 50381 | HLA-DQB1 | MHCII, DQ | major | 1.0 | 1.0 | 2.0 | 1.7 | 1.2 | 1.0 | [symbol] | 1.6 | 1.0 | 1.0 | 5.3 |
| | | beta 1 | histocompatibility | | | | | | | | | | | |
| | | | complex, class II, | | | | | | | | | | | |
| | | | DQ bet | | | | | | | | | | | |
| 50653 | GM2A | | | 0.9 | 1.0 | 1.7 | 1.6 | 1.1 | 1.0 | [symbol] | 1.6 | 1.2 | 1.1 | 5.3 |
| 47605 | HRMT1L2 | | | 1.3 | 1.2 | 0.5 | 1.8 | 0.8 | 1.1 | 0.6 | 1.4 | 0.9 | 1.0 | 5.3 |
| 48367 | SERPINE2 | | serine (or | 27.1 | 12.5 | 0.4 | 2.1 | 1.0 | 1.0 | 0.7 | 1.2 | 1.1 | 1.0 | 5.3 |
| | | | cysteine) | | | | | | | | | | | |
| | | | proteinase | | | | | | | | | | | |
| | | | inhibitor, clade E | | | | | | | | | | | |
| 50526 | HMGIY | HMG-I/Y | high-mobility | 0.7 | 1.4 | 0.5 | 1.6 | 0.9 | 1.1 | 0.6 | 1.4 | 0.8 | 1.1 | 5.2 |
| | | | group (nonhistone | | | | | | | | | | | |
| | | | chromosomal) | | | | | | | | | | | |
| | | | prote | | | | | | | | | | | |
| 50170 | EMILIN | EMILIN | elastin microfibril | 0.8 | 1.1 | 1.4 | 1.4 | 1.1 | 1.1 | [symbol] | 1.6 | 1.2 | 1.2 | 5.2 |
| | | | interface located | | | | | | | | | | | |
| | | | protein | | | | | | | | | | | |
| 47916 | SCYA2 | MCP1 | small inducible | 16.9 | 10.7 | 0.3 | 2.1 | 1.2 | 1.1 | 0.7 | 1.0 | 1.1 | 1.0 | 5.2 |
| | | | cytokine A2 | | | | | | | | | | | |
| | | | (monocyte | | | | | | | | | | | |
| | | | chemotactic | | | | | | | | | | | |
| 49117 | SERPINB1 | | serine (or | 3.8 | 2.9 | 0.7 | 1.2 | 1.0 | 1.0 | [symbol] | 1.9 | 1.2 | 1.0 | 5.2 |
| | | | cysteine) | | | | | | | | | | | |
| | | | proteinase | | | | | | | | | | | |
| | | | inhibitor, clade B | | | | | | | | | | | |
| 47137 | MD-2 | MD-2 | MD-2 protein | 1.0 | 1.0 | 2.1 | 1.7 | 1.1 | 1.0 | 1.6 | 1.4 | 1.2 | 1.2 | 5.2 |
| 49752 | | | | 2.0 | 1.8 | 0.5 | 2.0 | 0.9 | 1.0 | 0.7 | 1.2 | 1.1 | 1.0 | 5.2 |
| 47385 | FLJ10718 | | | 3.2 | 2.7 | 0.4 | 1.9 | 0.7 | 1.3 | 0.8 | 1.0 | 1.0 | 1.0 | 5.2 |
| 47505 | COL9A2 | COL9A2 | collagen, type IX, | 1.0 | 1.0 | 1.8 | 1.5 | 1.2 | 1.1 | [symbol] | 1.5 | 1.1 | 1.1 | 5.2 |
| | | | alpha 2 | | | | | | | | | | | |
| 48038 | MMP13 | MMP13 | matrix | 0.9 | 1.0 | 1.6 | 1.4 | 1.1 | 1.0 | [symbol] | 1.7 | 1.1 | 1.0 | 5.2 |
| | | | metalloproteinase | | | | | | | | | | | |
| | | | 13 (collagenase 3) | | | | | | | | | | | |
| 49904 | KIAA1557 | | | 2.3 | 1.7 | 2.3 | 2.1 | 1.1 | 1.0 | 1.2 | 1.1 | 0.9 | 1.0 | 5.2 |
| 46586 | C12orf8 | | | 0.9 | 1.0 | 1.7 | 1.3 | 1.2 | 1.1 | [symbol] | 1.8 | 1.2 | 1.1 | 5.2 |
| 49417 | HSPA1B | HSPA1B | heat shock 70 kD | 2.2 | 1.4 | 1.7 | 1.5 | 1.1 | 1.0 | [symbol] | 1.6 | 1.3 | 1.1 | 5.2 |
| | | | protein 1B | | | | | | | | | | | |
| 49044 | SCYA18 | DC-CK1 | small inducible | 26.3 | 23.0 | 0.4 | 1.7 | 0.6 | 1.4 | 1.3 | 1.1 | 1.0 | 1.0 | 5.2 |
| | | | cytokine subfamily | | | | | | | | | | | |
| | | | A (Cys—Cys), me | | | | | | | | | | | |
| 49870 | IFIT4 | | | 10.9 | 6.3 | 1.1 | 1.0 | 0.7 | 1.2 | [symbol] | 1.7 | 1.2 | 1.2 | 5.2 |
| 46748 | PTX3 | | | 8.7 | 3.4 | 1.4 | 1.3 | 1.0 | 1.0 | [symbol] | 1.5 | [symbol] | 1.3 | 5.2 |
| 50499 | RGS14 | RGS14 | regulator of G- | 0.4 | 2.3 | 2.0 | 1.7 | 1.3 | 1.2 | 1.4 | 1.3 | 1.0 | 1.0 | 5.2 |
| | | | protein signalling | | | | | | | | | | | |
| | | | 14 | | | | | | | | | | | |
| 50369 | SCYA3 | MIP1, alpha | small inducible | 8.5 | 8.4 | 0.4 | 2.0 | 0.8 | 1.0 | 1.2 | 1.1 | 1.2 | 1.0 | 5.2 |
| | | homolog | cytokine A3 | | | | | | | | | | | |
| | | | (homologous to | | | | | | | | | | | |
| | | | mouse M | | | | | | | | | | | |
| 49649 | FGF2 | FGF-2 | fibroblast growth | 3.0 | 2.8 | 1.0 | 1.0 | 0.9 | 1.0 | [symbol] | 2.1 | 0.9 | 1.0 | 5.1 |
| 49873 | INSIG1 | | | 1.5 | 1.4 | 0.6 | 1.4 | 0.7 | 1.4 | 1.0 | 1.0 | [symbol] | 1.3 | 5.1 |
| 47185 | HSPA1B | HSPA1B | heat shock 70 kD | 2.0 | 1.7 | 1.6 | 1.5 | 1.1 | 1.0 | [symbol] | 1.5 | 1.3 | 1.2 | 5.1 |
| | | | protein 1B | | | | | | | | | | | |
| 48971 | P4HA2 | P4HA2 | procollagen- | 1.2 | 1.1 | 2.0 | 1.7 | 1.0 | 1.0 | 1.7 | 1.4 | 1.0 | 1.0 | 5.1 |
| | | | proline, 2- | | | | | | | | | | | |
| | | | oxoglutarate 4- | | | | | | | | | | | |
| | | | dioxygenase | | | | | | | | | | | |
| 48946 | FGFR1 | FGF receptor 1 | fibroblast growth | 0.7 | 1.3 | 1.7 | 1.6 | 1.0 | 1.0 | [symbol] | 1.5 | 1.0 | 1.0 | 5.1 |
| | | | factor receptor 1 | | | | | | | | | | | |
| | | | (fms-related t | | | | | | | | | | | |
| 49812 | TXNIP | VDUP1 | upregulated by | 0.6 | 1.2 | 1.5 | 1.2 | 1.2 | 1.2 | [symbol] | 1.7 | 1.1 | 1.0 | 5.1 |
| | | | 1,25- | | | | | | | | | | | |
| | | | dihydroxyvitamin | | | | | | | | | | | |
| | | | D-3 | | | | | | | | | | | |
| 48000 | CTSH | | | 0.8 | 1.1 | 1.7 | 1.6 | 1.1 | 1.1 | 1.6 | 1.4 | 1.1 | 1.0 | 5.1 |
| 50433 | GNAS | G protein, | guanine | 0.9 | 1.1 | 1.6 | 1.4 | 1.1 | 1.0 | [symbol] | 1.7 | 1.1 | 1.0 | 5.1 |
| | | alpha s1 | nucleotide binding | | | | | | | | | | | |
| | | | protein (G | | | | | | | | | | | |
| | | | protein), al | | | | | | | | | | | |
| 50200 | PTGER4 | EP2 | prostaglandin E | 1.4 | 1.0 | 0.7 | 1.3 | 1.1 | 1.1 | [symbol] | 1.6 | 1.3 | 1.2 | 5.1 |
| | | | receptor 4 | | | | | | | | | | | |
| | | | (subtype EP4) | | | | | | | | | | | |
| 46930 | CD14 | CD14 | CD14 antigen | 0.6 | 1.2 | 2.4 | 2.0 | 1.1 | 1.1 | 1.2 | 1.0 | 0.8 | 1.0 | 5.1 |
| 49389 | DUSP1 | MKP-1, | dual specificity | 5.5 | 5.4 | 0.6 | 1.6 | 0.9 | 1.1 | 0.7 | 1.3 | 0.8 | 1.2 | 5.1 |
| | | PTPN10 | phosphatase 1 | | | | | | | | | | | |
| 48185 | XAGE-1 | | | 1.0 | 1.0 | 1.4 | 1.3 | 1.0 | 1.0 | [symbol] | 1.7 | 1.2 | 1.1 | 5.1 |
| 48009 | ST14 | ST14 | suppression of | 0.4 | 1.9 | 1.6 | 1.4 | 1.2 | 1.1 | [symbol] | 1.6 | 1.2 | 1.1 | 5.1 |
| | | | tumorigenicity 14 | | | | | | | | | | | |
| | | | (colon carcinoma, | | | | | | | | | | | |
| 50009 | SCYA3 | MIP1, alpha | small inducible | 7.8 | 5.0 | 0.4 | 1.8 | 0.8 | 1.0 | 1.2 | 1.2 | 1.2 | 1.1 | 5.1 |
| | | homolog | cytokine A3 | | | | | | | | | | | |
| | | | (homologous to | | | | | | | | | | | |
| | | | mouse M | | | | | | | | | | | |
| 47551 | PTGER4 | EP2 | prostaglandin E | 1.4 | 1.3 | 0.7 | 1.3 | 1.1 | 1.0 | [symbol] | 1.5 | [symbol] | 1.3 | 5.1 |
| | | | receptor 4 | | | | | | | | | | | |
| | | | (subtype EP4) | | | | | | | | | | | |
| 47118 | PLA2G4A | phospholipase | phospholipase A2, | 2.0 | 1.6 | 1.3 | 1.2 | 1.0 | 1.0 | [symbol] | 1.6 | [symbol] | 1.3 | 5.1 |
| | | A2 | group IVA | | | | | | | | | | | |
| | | | (cytosolic, | | | | | | | | | | | |
| | | | calcium-de | | | | | | | | | | | |
| 48013 | CEP3 | CEP3 | Cdc42 effector | 1.4 | 1.3 | 1.4 | 1.2 | 1.0 | 1.0 | [symbol] | 1.8 | 1.2 | 1.1 | 5.1 |
| | | | protein 3 | | | | | | | | | | | |
| 46955 | EREG | epiregulin | epiregulin | 3.8 | 3.5 | 1.0 | 1.0 | 1.0 | 1.0 | [symbol] | 2.1 | 0.8 | 1.0 | 5.1 |
| 50341 | ST14 | ST14 | suppression of | 0.4 | 1.8 | 1.6 | 1.5 | 1.2 | 1.1 | [symbol] | 1.6 | 1.1 | 1.0 | 5.1 |
| | | | tumorigenicity 14 | | | | | | | | | | | |
| | | | (colon carcinoma, | | | | | | | | | | | |
| 47699 | PAK1 | Pak1 | p21/Cdc42/Rac1- | 0.9 | 1.1 | 2.0 | 1.8 | 1.0 | 1.0 | 1.4 | 1.3 | 1.0 | 1.0 | 5.1 |
| | | | activated kinase 1 | | | | | | | | | | | |
| | | | (yeast Ste20-rel | | | | | | | | | | | |
| 47499 | PTGS2 | PTGS2, COX2 | prostaglandin- | 28.8 | 28.0 | 0.7 | 1.0 | 0.9 | 1.0 | [symbol] | 1.9 | 1.3 | 1.1 | 5.1 |
| | | | endoperoxide | | | | | | | | | | | |
| | | | synthase 2 | | | | | | | | | | | |
| | | | (prostagland | | | | | | | | | | | |
| 48543 | C2 | | | 1.9 | 1.7 | 2.0 | 1.6 | 1.3 | 1.2 | 1.3 | 1.1 | 1.2 | 1.2 | 5.1 |
| 49617 | FOXF1 | FOXF1 | forkhead box F1 | 3.3 | 1.7 | 1.4 | 1.1 | 1.0 | 1.0 | [symbol] | 1.8 | 1.3 | 1.2 | 5.1 |
| 47580 | EMS1 | cortactin | ems1 sequence | 0.8 | 1.1 | 0.6 | 1.5 | 1.0 | 1.0 | [symbol] | 1.6 | 1.0 | 1.0 | 5.1 |
| | | | (mammary tumor | | | | | | | | | | | |
| | | | and squamous | | | | | | | | | | | |
| | | | cell car | | | | | | | | | | | |
| 48468 | NRG1 | neuregulin1, | neuregulin 1 | 5.3 | 4.1 | 0.5 | 1.6 | 0.7 | 1.3 | 0.7 | 1.0 | 0.8 | 1.1 | 5.1 |
| | | neu diff. | | | | | | | | | | | | |
| | | Factor, | | | | | | | | | | | | |
| | | heregulin | | | | | | | | | | | | |
| 48585 | SCYA3 | MIP1, alpha | small inducible | 9.5 | 8.6 | 0.5 | 1.8 | 0.8 | 1.1 | 1.2 | 1.2 | 1.2 | 1.0 | 5.1 |
| | | homolog | cytokine A3 | | | | | | | | | | | |
| | | | (homologous to | | | | | | | | | | | |
| | | | mouse M | | | | | | | | | | | |
| 49416 | S100A10 | S100A10 | S100 calcium- | 1.2 | 1.0 | 1.9 | 1.5 | 1.1 | 1.0 | 1.9 | 1.4 | 1.2 | 1.1 | 5.1 |
| | | | binding protein | | | | | | | | | | | |
| | | | A10 (annexin II | | | | | | | | | | | |
| | | | ligan | | | | | | | | | | | |
| 47139 | SERPINI1 | | serine (or | 1.0 | 1.0 | 2.2 | 1.4 | 1.1 | 1.1 | [symbol] | 1.6 | 1.1 | 1.0 | 5.1 |
| | | | cysteine) | | | | | | | | | | | |
| | | | proteinase | | | | | | | | | | | |
| | | | inhibitor, clade I | | | | | | | | | | | |
| 48257 | SCYA4 | MIP-1beta | small inducible | 40.5 | 17.9 | 0.3 | 1.8 | 0.9 | 1.0 | 1.2 | 1.2 | 1.2 | 1.0 | 5.0 |
| | | | cytokine A4 | | | | | | | | | | | |
| | | | (homologous to | | | | | | | | | | | |
| | | | mouse M | | | | | | | | | | | |
| 50126 | EREG | epiregulin | epiregulin | 6.6 | 6.2 | 1.0 | 1.0 | 0.7 | 1.0 | [symbol] | 1.9 | 0.8 | 1.1 | 5.0 |
| 47056 | NFRKB | | | 0.8 | 1.2 | 0.6 | 1.5 | 0.9 | 1.0 | 0.6 | 1.4 | 0.9 | 1.1 | 5.0 |
| 47070 | CPD | | | 1.7 | 1.6 | 1.5 | 1.4 | 1.1 | 1.0 | [symbol] | 1.6 | 1.1 | 1.1 | 5.0 |
| 49054 | ITGA2 | integrin, alpha | integrin, alpha 2 | 0.9 | 1.0 | 0.6 | 1.4 | 0.9 | 1.0 | [symbol] | 1.6 | 1.0 | 1.0 | 5.0 |
| | | 2, CD49B, | (CD49B, alpha 2 | | | | | | | | | | | |
| | | VLA-2 | subunit of VLA-2 | | | | | | | | | | | |
| 50338 | PIM2 | Pim-2 | pim-2 oncogene | 4.7 | 2.8 | 1.3 | 1.2 | 1.0 | 1.0 | [symbol] | 1.8 | 1.1 | 1.0 | 5.0 |
| 48290 | NEDD4L | | | 1.6 | 1.5 | 0.6 | 1.4 | 0.8 | 1.1 | [symbol] | 1.5 | 0.8 | 1.1 | 5.0 |
| 50349 | LOC51295 | ECSIT | ECSIT | 0.7 | 1.3 | 0.6 | 1.5 | 1.0 | 1.0 | [symbol] | 1.5 | 0.9 | 1.0 | 5.0 |
| 49016 | LIPA | | | 1.1 | 1.0 | 1.4 | 1.3 | 1.1 | 1.0 | [symbol] | 1.6 | 1.2 | 1.1 | 5.0 |
| 48609 | SERPINF1 | | serine (or | 0.8 | 1.1 | 1.5 | 1.3 | 1.1 | 1.0 | [symbol] | 1.7 | 1.1 | 1.0 | 5.0 |
| | | | cysteine) | | | | | | | | | | | |
| | | | proteinase | | | | | | | | | | | |
| | | | inhibitor, clade F | | | | | | | | | | | |
| 46796 | STC1 | | | 2.3 | 1.7 | 0.8 | 1.1 | 0.8 | 1.0 | [symbol] | 1.5 | [symbol] | 1.3 | 5.0 |
| 47926 | CCNE2 | cyclin E2 | cyclin E2 | 1.6 | 1.2 | 0.5 | 1.8 | 1.0 | 1.0 | 0.6 | 1.2 | 1.2 | 1.0 | 5.0 |
| 50493 | HMGIY | HMG-I/Y | high-mobility | 0.7 | 1.4 | 0.6 | 1.4 | 0.9 | 1.1 | 0.6 | 1.4 | 0.8 | 1.1 | 5.0 |
| | | | group (nonhistone | | | | | | | | | | | |
| | | | chromosomal) | | | | | | | | | | | |
| | | | prote | | | | | | | | | | | |
| 49839 | SCYB5 | ENA-78 | small inducible | 2.6 | 1.9 | 0.8 | 1.2 | 0.8 | 1.0 | 0.5 | 1.3 | [symbol] | 1.4 | 5.0 |
| | | | cytokine subfamily | | | | | | | | | | | |
| | | | B (Cys-X-Cys), | | | | | | | | | | | |
| 50199 | | | | 1.1 | 1.0 | 1.9 | 1.8 | 1.0 | 1.0 | 1.5 | 1.2 | 1.0 | 1.0 | 5.0 |
| 49679 | SERPINF1 | | serine (or | 0.8 | 1.2 | 1.6 | 1.4 | 1.1 | 1.1 | [symbol] | 1.5 | 1.1 | 1.0 | 5.0 |
| | | | cysteine) | | | | | | | | | | | |
| | | | proteinase | | | | | | | | | | | |
| | | | inhibitor, clade F | | | | | | | | | | | |
| 50311 | IFNGR1 | IFN gamma | interferon gamma | 0.7 | 1.2 | 2.3 | 1.8 | 1.2 | 1.0 | 1.3 | 1.1 | 0.9 | 1.0 | 5.0 |
| | | receptor 1 | receptor 1 | | | | | | | | | | | |
| 50347 | TRAP1 | TRAP1, hsp90 | heat shock protein | 0.7 | 1.3 | 0.6 | 1.4 | 0.9 | 1.0 | [symbol] | 1.6 | 0.9 | 1.0 | 5.0 |
| | | related | 75 | | | | | | | | | | | |
| 50343 | JAK2 | JAK2 | Janus kinase 2 (a | 4.6 | 4.2 | 1.4 | 1.2 | 1.0 | 1.0 | [symbol] | 1.5 | 1.3 | 1.2 | 5.0 |
| | | | protein tyrosine | | | | | | | | | | | |
| | | | kinase) | | | | | | | | | | | |
| 49132 | CASP1 | caspase 1, | caspase 1, | 4.1 | 3.0 | 1.9 | 1.4 | 1.1 | 1.1 | 1.5 | 1.3 | 1.2 | 1.2 | 5.0 |
| | | ICE | apoptosis-related | | | | | | | | | | | |
| | | | cysteine protease | | | | | | | | | | | |
| | | | (in | | | | | | | | | | | |
| 47328 | PPFIBP1 | PPFIBP1 | PTPRF interacting | 0.6 | 1.5 | 0.6 | 1.6 | 0.9 | 1.0 | 0.7 | 1.3 | 0.9 | 1.0 | 5.0 |
| | | | protein, binding | | | | | | | | | | | |
| | | | protein 1 (lipr | | | | | | | | | | | |
| 48771 | | | | 0.9 | 1.1 | 1.5 | 1.4 | 0.9 | 1.0 | [symbol] | 1.5 | 1.0 | 1.0 | 5.0 |
| 50046 | DUSP1 | MKP-1, | dual specificity | 5.6 | 5.0 | 0.5 | 1.8 | 0.9 | 1.0 | 0.7 | 1.2 | 0.9 | 1.0 | 5.0 |
| | | PTPN10 | phosphatase 1 | | | | | | | | | | | |
| 49779 | TXNIP | VDUP1 | upregulated by | 0.6 | 1.4 | 1.5 | 1.2 | 1.2 | 1.1 | [symbol] | 1.6 | 1.1 | 1.1 | 5.0 |
| | | | 1,25- | | | | | | | | | | | |
| | | | dihydroxyvitamin | | | | | | | | | | | |
| | | | D-3 | | | | | | | | | | | |
| 47969 | MAP3K8 | c-cot | mitogen-activated | 4.9 | 4.8 | 0.9 | 1.0 | 0.8 | 1.0 | [symbol] | 1.8 | 1.4 | 1.1 | 5.0 |
| | | | protein kinase | | | | | | | | | | | |
| | | | kinase kinase 8 | | | | | | | | | | | |
| 47386 | GALNAC4S-6ST | BRAG | B cell RAG | 0.7 | 1.0 | 0.6 | 1.5 | 1.0 | 1.0 | 0.6 | 1.4 | 0.8 | 1.1 | 5.0 |
| | | | associated protein | | | | | | | | | | | |

APPENDIX C
AA Pathway Inhibitors
Genes differentially expressed (≧1.3-fold) in A549 cells in response to at least one arachidonic acid pathway inhibitor, in presence of IL-1β
Genes in bold have min fold change ≧1.3

| | | | | | | | +IL1b +/− Triac, | | +IL1b +/− Indo, |
|---|---|---|---|---|---|---|---|---|---|
| | | | | +IL1b +/− Dexa, | +IL1b +/− Dexa, | +IL1b +/− Triac, | min | +IL1b +/− Indo, | min |
| | | | | wtd | min fold | wtd | fold | wtd | fold |
| SpotID | GeneSymbol | GeneName | GeneAssignment | means | change | means | change | means | change |
| 48626 | | | | 1.89 | 1.67 | 2.09 | 1.79 | 1.86 | 1.77 |
| 47003 | | | | 1.81 | 1.41 | 2.01 | 1.59 | 1.84 | 1.68 |
| 47624 | | | | 1.61 | 1.48 | 1.97 | 1.74 | 1.80 | 1.64 |
| 50456 | | | | 1.54 | 1.24 | 1.90 | 1.53 | 1.70 | 1.59 |
| 46987 | | | | 2.82 | 2.22 | 2.05 | 1.57 | 1.77 | 1.53 |
| 50468 | | | | 1.45 | 1.23 | 1.74 | 1.47 | 1.72 | 1.52 |
| 46609 | | | | 1.65 | 1.30 | 1.82 | 1.67 | 1.71 | 1.52 |
| 49817 | | | | 1.47 | 1.26 | 2.06 | 1.75 | 1.76 | 1.48 |
| 49343 | | | | 1.76 | 1.62 | 1.94 | 1.60 | 1.77 | 1.45 |
| 46594 | | | | 1.64 | 1.05 | 1.73 | 1.62 | 1.77 | 1.38 |
| 47206 | | | | 1.79 | 1.12 | 1.91 | 1.60 | 1.58 | 1.35 |
| 48580 | | | | 1.69 | 1.44 | 1.50 | 1.47 | 1.47 | 1.32 |
| 47048 | | | | 2.19 | 1.26 | 2.17 | 1.66 | 1.58 | 1.31 |
| 48763 | | | | 1.72 | 1.47 | 1.54 | 1.31 | 1.25 | 1.17 |
| 48036 | | | | 1.63 | 1.38 | 1.68 | 1.51 | 1.39 | 1.13 |
| 50596 | | | | 2.04 | 1.75 | 0.81 | 1.07 | 0.77 | 1.12 |
| 49612 | | | | 1.63 | 1.34 | 1.98 | 1.54 | 1.37 | 1.11 |
| 47315 | | | | 1.50 | 1.21 | 1.70 | 1.47 | 1.52 | 1.09 |
| 50250 | | | putative methyltransferase | 1.48 | 1.03 | 1.41 | 1.36 | 1.13 | 1.09 |
| 48260 | | | | 1.72 | 1.23 | 2.14 | 1.75 | 1.56 | 1.07 |
| 49348 | | | KIAA1085 protein | 1.19 | 1.04 | 1.39 | 1.34 | 1.11 | 1.05 |
| 47016 | | LBC, lymphoid | lymphoid blast crisis oncogene | 1.51 | 1.36 | 1.19 | 1.10 | 1.17 | 1.05 |
| | | blast crisis | | | | | | | |
| | | oncogene | | | | | | | |
| 46753 | | | | 1.38 | 1.00 | 1.44 | 1.36 | 1.08 | 1.05 |
| 48924 | | | | 0.96 | 1.00 | 1.58 | 1.40 | 1.19 | 1.05 |
| 46769 | | | | 1.53 | 1.10 | 1.83 | 1.70 | 1.41 | 1.02 |
| 47233 | | | | 1.72 | 1.53 | 0.46 | 1.71 | 0.98 | 1.00 |
| 48039 | | | | 0.76 | 1.25 | 1.73 | 1.54 | 0.92 | 1.00 |
| 46783 | | | | 0.84 | 1.08 | 2.02 | 1.46 | 1.09 | 1.00 |
| 50624 | | | | 2.08 | 1.46 | 2.27 | 1.43 | 1.08 | 1.00 |
| 48604 | | | | 0.30 | 2.29 | 1.48 | 1.40 | 1.00 | 1.00 |
| 46580 | | | | 1.30 | 1.00 | 1.52 | 1.39 | 1.24 | 1.00 |
| 48060 | | | | 0.23 | 2.64 | 1.39 | 1.27 | 1.04 | 1.00 |
| 50375 | | LBC, lymphoid | lymphoid blast crisis oncogene | 3.55 | 3.24 | 1.25 | 1.24 | 1.06 | 1.00 |
| | | blast crisis | | | | | | | |
| | | oncogene | | | | | | | |
| 49276 | | | | 0.48 | 1.69 | 0.67 | 1.23 | 0.90 | 1.00 |
| 49147 | | | | 0.47 | 1.83 | 0.69 | 1.20 | 0.83 | 1.00 |
| 50287 | | | | 0.51 | 1.76 | 0.78 | 1.18 | 0.85 | 1.00 |
| 48336 | | | | 0.44 | 1.35 | 1.22 | 1.15 | 1.02 | 1.00 |
| 49908 | | | | 1.74 | 1.59 | 1.94 | 1.12 | 1.22 | 1.00 |
| 49425 | | | | 0.49 | 1.67 | 1.23 | 1.10 | 1.06 | 1.00 |
| 46627 | | | | 1.58 | 1.37 | 1.30 | 1.04 | 1.13 | 1.00 |
| 48775 | | | | 0.71 | 1.32 | 0.91 | 1.02 | 0.97 | 1.00 |
| 50562 | | | | 2.46 | 2.01 | 1.14 | 1.00 | 0.84 | 1.00 |
| 50782 | | | | 1.68 | 1.58 | 0.95 | 1.00 | 1.02 | 1.00 |
| 48047 | | | | 1.78 | 1.45 | 1.10 | 1.00 | 1.06 | 1.00 |
| 50151 | | | | 0.63 | 1.31 | 0.99 | 1.00 | 0.94 | 1.00 |
| 49051 | A2M | | alpha-2-macroglobulin | 0.51 | 1.54 | 1.00 | 1.00 | 1.00 | 1.00 |
| 49952 | AADAC | | arylacetamide deacetylase (esterase) | 0.52 | 1.41 | 1.50 | 1.40 | 0.91 | 1.00 |
| 50633 | ABL2 | | v-abl Abelson murine leukemia viral oncogene | 1.27 | 1.00 | 1.73 | 1.49 | 1.09 | 1.00 |
| | | | homolog 2 (arg, Abelson-related gene) | | | | | | |
| 47263 | ACVR2 | | activin A receptor, type II | 1.51 | 1.24 | 1.67 | 1.58 | 1.27 | 1.16 |
| 48251 | ADAM15 | ADAM15 | a disintegrin and metalloproteinase domain 15 | 1.40 | 1.33 | 0.77 | 1.21 | 0.92 | 1.00 |
| | | | (metargidin) | | | | | | |
| 49237 | ADORA2B | | adenosine A2b receptor | 0.63 | 1.33 | 1.08 | 1.00 | 0.88 | 1.00 |
| 47060 | AF1Q | | ALL1-fused gene from chromosome 1q | 0.43 | 2.17 | 1.36 | 1.30 | 0.91 | 1.00 |
| 50345 | AGT | | angiotensinogen (serine (or cysteine) | 2.59 | 2.45 | 1.00 | 1.00 | 0.98 | 1.00 |
| | | | proteinase inhibitor, clade A (alpha-1 | | | | | | |
| | | | antiproteinase, antitrypsin), member 8) | | | | | | |
| 49620 | AKAP1 | | A kinase (PRKA) anchor protein 1 | 1.61 | 1.38 | 0.85 | 1.06 | 1.02 | 1.00 |
| 48794 | ANXA3 | annexin III | annexin A3 | 1.14 | 1.00 | 1.36 | 1.31 | 1.09 | 1.00 |
| 49549 | ANXA8 | | annexin A8 | 1.60 | 1.39 | 1.20 | 1.11 | 0.89 | 1.00 |
| 49524 | AP1G2 | | adaptor-related protein complex 1, gamma 2 | 1.10 | 1.00 | 1.39 | 1.30 | 0.99 | 1.00 |
| | | | subunit | | | | | | |
| 49935 | APOA2 | | apolipoprotein A-II | 0.28 | 2.55 | 1.03 | 1.00 | 0.90 | 1.00 |
| 46949 | AREG | amphiregulin | amphiregulin (schwannoma-derived growth | 0.09 | 10.54 | 0.75 | 1.17 | 0.90 | 1.09 |
| | | | factor) | | | | | | |
| 46953 | AREG | amphiregulin | amphiregulin (schwannoma-derived growth | 0.09 | 10.74 | 0.76 | 1.19 | 0.89 | 1.08 |
| | | | factor) | | | | | | |
| 46982 | AREG | amphiregulin | amphiregulin (schwannoma-derived growth | 0.09 | 10.64 | 0.75 | 1.22 | 0.94 | 1.00 |
| | | | factor) | | | | | | |
| 47617 | ARF3 | | ADP-ribosylation factor 3 | 1.08 | 1.00 | 1.62 | 1.44 | 1.01 | 1.00 |
| 50502 | ARF6 | ARF6 | ADP-ribosylation factor 6 | 1.50 | 1.32 | 0.88 | 1.07 | 0.93 | 1.00 |
| 48357 | ARF6 | ARF6 | ADP-ribosylation factor 6 | 1.63 | 1.41 | 0.95 | 1.00 | 0.90 | 1.00 |
| 50469 | ARF6 | ARF6 | ADP-ribosylation factor 6 | 1.47 | 1.30 | 0.94 | 1.00 | 0.93 | 1.00 |
| 47192 | ARHGAP1 | ARHGAP1 | Rho GTPase activating protein 1 | 1.44 | 1.36 | 1.54 | 1.44 | 1.21 | 1.07 |
| 46648 | ARHGAP1 | ARHGAP1 | Rho GTPase activating protein 1 | 1.41 | 1.35 | 1.54 | 1.41 | 1.24 | 1.02 |
| 46873 | ARHGAP4 | ARHGAP4 | Rho GTPase activating protein 4 | 0.85 | 1.00 | 1.65 | 1.40 | 1.12 | 1.00 |
| 48984 | ARHGDIB | ARHGDIB | Rho GDP dissociation inhibitor (GDI) beta | 0.48 | 1.47 | 1.22 | 1.05 | 0.99 | 1.00 |
| 48287 | ARHGEF15 | ARHGEF15 | Rho guanine nucleotide exchange factor (GEF) | 1.63 | 1.32 | 1.87 | 1.45 | 1.83 | 1.37 |
| | | | 15 | | | | | | |
| 48322 | ARHGEF16 | ARHGEF16 | Rho guanine exchange factor (GEF) 16 | 1.43 | 1.00 | 1.73 | 1.53 | 1.46 | 1.00 |
| 48277 | ARHGEF2 | ARHGEF2 | rho/rac guanine nucleotide exchange factor | 0.41 | 2.22 | 1.18 | 1.05 | 0.91 | 1.00 |
| | | | (GEF) 2 | | | | | | |
| 48346 | ARHGEF2 | ARHGEF2 | rho/rac guanine nucleotide exchange factor | 0.48 | 1.81 | 1.18 | 1.00 | 0.97 | 1.00 |
| | | | (GEF) 2 | | | | | | |
| 48516 | ARL6IP | | ADP-ribosylation factor-like 6 interacting | 0.84 | 1.07 | 0.62 | 1.41 | 0.86 | 1.04 |
| | | | protein | | | | | | |
| 50440 | ATF5 | ATF5 | activating transcription factor 5 | 1.37 | 1.00 | 1.53 | 1.49 | 1.14 | 1.06 |
| 47568 | ATF7 | ATF7 | activating transcription factor 7 | 1.73 | 1.24 | 1.92 | 1.53 | 1.66 | 1.42 |
| 50635 | B4GALT1 | | UDP-Gal: betaGlcNAc beta 1,4- | 1.58 | 1.49 | 1.10 | 1.00 | 0.98 | 1.00 |
| | | | galactosyltransferase, polypeptide 1 | | | | | | |
| 49146 | BAG4 | BAG-4, SODD | BCL2-associated athanogene 4 | 0.60 | 1.38 | 0.89 | 1.00 | 1.16 | 1.00 |
| 49307 | BAG5 | BAG5 | BCL2-associated athanogene 5 | 1.40 | 1.00 | 1.57 | 1.36 | 1.19 | 1.00 |
| 48802 | BAI3 | | brain-specific angiogenesis inhibitor 3 | 0.69 | 1.38 | 0.93 | 1.03 | 0.95 | 1.01 |
| 47691 | BATF | B-ATF | basic leucine zipper transcription factor, ATF- | 1.30 | 1.00 | 1.54 | 1.35 | 1.15 | 1.00 |
| | | | like | | | | | | |
| 49013 | BBC3 | | Bcl-2 binding component 3 | 0.66 | 1.34 | 0.99 | 1.00 | 0.97 | 1.00 |
| 47650 | BBS2 | BBS2 | Bardet-Biedl syndrome 2 | 1.44 | 1.34 | 1.05 | 1.00 | 0.97 | 1.00 |
| 50686 | BCL2 | Bcl2 | B-cell CLL/lymphoma 2 | 1.31 | 1.00 | 1.62 | 1.37 | 1.17 | 1.00 |
| 50019 | BCL2A1 | BCL2A1 | BCL2-related protein A1 | 1.10 | 1.00 | 1.43 | 1.34 | 1.13 | 1.07 |
| 47738 | BCL2A1 | BCL2A1 | BCL2-related protein A1 | 0.25 | 3.26 | 1.29 | 1.19 | 1.06 | 1.00 |
| 48679 | BCL3 | Bcl3 | B-cell CLL/lymphoma 3 | 1.48 | 1.20 | 1.50 | 1.45 | 1.43 | 1.21 |
| 48374 | BDKRB1 | BDKRB1 | bradykinin receptor B1 | 0.16 | 4.59 | 1.18 | 1.13 | 0.93 | 1.02 |
| 47979 | BDKRB1 | BDKRB1 | bradykinin receptor B1 | 0.27 | 3.24 | 1.21 | 1.09 | 0.95 | 1.00 |
| 46689 | BENE | | BENE protein | 1.01 | 1.00 | 0.62 | 1.36 | 0.97 | 1.00 |
| 47396 | BHLHB2 | | basic helix-loop-helix domain containing, class | 0.53 | 1.60 | 0.99 | 1.00 | 0.96 | 1.00 |
| | | | B, 2 | | | | | | |
| 50726 | BID | BID | BH3 interacting domain death agonist | 0.69 | 1.33 | 0.80 | 1.12 | 0.97 | 1.01 |
| 48408 | BIRC2 | cIAP1 | baculoviral IAP repeat-containing 2 | 0.64 | 1.43 | 0.91 | 1.05 | 1.02 | 1.00 |
| 49061 | BIRC2 | cIAP1 | baculoviral IAP repeat-containing 2 | 0.60 | 1.41 | 0.93 | 1.03 | 0.99 | 1.00 |
| 49094 | BIRC2 | cIAP1 | baculoviral IAP repeat-containing 2 | 0.60 | 1.47 | 0.92 | 1.00 | 1.05 | 1.00 |
| 49840 | BMP4 | BMP4 | bone morphogenetic protein 4 | 0.24 | 3.58 | 0.49 | 1.92 | 0.98 | 1.00 |
| 49098 | BMP4 | BMP4 | bone morphogenetic protein 4 | 0.33 | 1.65 | 0.38 | 1.90 | 0.94 | 1.00 |
| 49065 | BMP4 | BMP4 | bone morphogenetic protein 4 | 0.18 | 3.21 | 0.40 | 1.79 | 0.98 | 1.00 |
| 49148 | BMP7 | BMP7 | bone morphogenetic protein 7 (osteogenic | 0.43 | 1.71 | 0.90 | 1.00 | 0.81 | 1.00 |
| | | | protein 1) | | | | | | |
| 49058 | BNIP3 | BNIP3 | BCL2/adenovirus E1B 19 kD interacting protein 3 | 0.56 | 1.47 | 1.25 | 1.14 | 0.95 | 1.00 |
| 46916 | BNIP3 | BNIP3 | BCL2/adenovirus E1B 19 kD interacting protein 3 | 0.56 | 1.75 | 1.36 | 1.13 | 0.92 | 1.00 |
| 49028 | BNIP3L | BNIP3L | BCL2/adenovirus E1B 19 kD interacting protein | 0.44 | 2.11 | 1.20 | 1.14 | 0.95 | 1.03 |
| | | | 3-like | | | | | | |
| 49301 | BPAG1 | BPAG1 | bullous pemphigoid antigen 1 (230/240 kD) | 0.96 | 1.00 | 1.62 | 1.50 | 1.36 | 1.15 |
| 50489 | BPAG1 | BPAG1 | bullous pemphigoid antigen 1 (230/240 kD) | 0.88 | 1.00 | 1.91 | 1.58 | 1.25 | 1.00 |
| 50522 | BPAG1 | BPAG1 | bullous pemphigoid antigen 1 (230/240 kD) | 0.84 | 1.00 | 1.63 | 1.35 | 1.20 | 1.00 |
| 47795 | BPHL | | biphenyl hydrolase-like (serine hydrolase; | 1.55 | 1.01 | 1.70 | 1.60 | 1.20 | 1.14 |
| | | | breast epithelial mucin-associated antigen) | | | | | | |
| 47516 | BTG1 | | B-cell translocation gene 1, anti-proliferative | 0.64 | 1.43 | 1.36 | 1.31 | 1.07 | 1.00 |
| 46854 | BTN3A2 | BTN3A2 | butyrophilin, subfamily 3, member A2 | 1.70 | 1.29 | 2.12 | 1.99 | 1.76 | 1.54 |
| 49509 | C3 | | complement component 3 | 2.75 | 2.65 | 0.86 | 1.00 | 1.02 | 1.00 |
| 49513 | CA3 | | carbonic anhydrase III, muscle specific | 0.55 | 1.47 | 0.99 | 1.00 | 1.09 | 1.00 |
| 48299 | CALM2 | | calmodulin 2 (phosphorylase kinase, delta) | 1.61 | 1.52 | 0.86 | 1.04 | 1.09 | 1.00 |
| 47637 | CALM2 | | calmodulin 2 (phosphorylase kinase, delta) | 1.61 | 1.55 | 0.85 | 1.02 | 1.04 | 1.00 |
| 49750 | CARD14 | CARD14 | caspase recruitment domain protein 14 | 0.97 | 1.00 | 1.61 | 1.38 | 0.97 | 1.00 |
| 49807 | CASP4 | CASP4 | caspase 4, apoptosis-related cysteine protease | 0.73 | 1.04 | 1.42 | 1.35 | 1.15 | 1.12 |
| 47007 | CASP4 | CASP4 | caspase 4, apoptosis-related cysteine protease | 0.81 | 1.08 | 1.45 | 1.37 | 1.15 | 1.09 |
| 50721 | CASP4 | CASP4 | caspase 4, apoptosis-related cysteine protease | 0.82 | 1.04 | 1.45 | 1.35 | 1.00 | 1.00 |
| 50372 | CASP6 | caspase-6 | caspase 6, apoptosis-related cysteine protease | 1.57 | 1.22 | 1.80 | 1.53 | 1.60 | 1.20 |
| 49909 | CASR | | calcium-sensing receptor (hypocalciuric | 0.54 | 1.65 | 1.42 | 1.33 | 1.14 | 1.12 |
| | | | hypercalcemia 1, severe neonatal | | | | | | |
| | | | hyperparathyroidism) | | | | | | |
| 49836 | CCL19 | MIP-3b | small inducible cytokine subfamily A (Cys—Cys), | 0.61 | 1.35 | 1.03 | 1.00 | 1.05 | 1.00 |
| | | | member 19 | | | | | | |
| 48830 | CCL2 | MCP1 | small inducible cytokine A2 (monocyte | 0.23 | 4.02 | 0.60 | 1.40 | 1.01 | 1.00 |
| | | | chemotactic protein 1) | | | | | | |
| 50026 | CCL2 | MCP1 | small inducible cytokine A2 (monocyte | 0.15 | 4.46 | 0.57 | 1.24 | 1.00 | 1.00 |
| | | | chemotactic protein 1) | | | | | | |
| 47916 | CCL2 | MCP1 | small inducible cytokine A2 (monocyte | 0.13 | 5.80 | 0.68 | 1.12 | 1.04 | 1.00 |
| | | | chemotactic protein 1) | | | | | | |
| 46596 | CCL20 | MIP-3a | small inducible cytokine subfamily A (Cys—Cys), | 0.60 | 1.59 | 0.91 | 1.04 | 0.92 | 1.03 |
| | | | member 20 | | | | | | |
| 48190 | CCL20 | MIP-3a | small inducible cytokine subfamily A (Cys—Cys), | 0.14 | 7.04 | 0.70 | 1.25 | 1.03 | 1.00 |
| | | | member 20 | | | | | | |
| 46598 | CCL20 | MIP-3a | small inducible cytokine subfamily A (Cys—Cys), | 0.10 | 9.88 | 0.76 | 1.18 | 0.95 | 1.00 |
| | | | member 20 | | | | | | |
| 49742 | CCL3 | MIP1, alpha | small inducible cytokine A3 | 0.57 | 1.00 | 2.15 | 1.88 | 1.13 | 1.00 |
| | | homolog | | | | | | | |
| 47893 | CCL4 | MIP-1beta | small inducible cytokine A4 | 0.58 | 1.31 | 1.11 | 1.00 | 0.79 | 1.04 |
| 47575 | CCL8 | MCP-2 | small inducible cytokine subfamily A (Cys—Cys), | 0.18 | 2.04 | 0.54 | 1.40 | 1.11 | 1.00 |
| | | | member 8 (monocyte chemotactic protein 2) | | | | | | |
| 49725 | CCM1 | | cerebral cavernous malformations 1 | 1.53 | 1.18 | 1.44 | 1.31 | 1.37 | 1.21 |
| 49085 | CCNA2 | cyclin A2 | cyclin A2 | 0.77 | 1.17 | 0.32 | 1.42 | 0.74 | 1.18 |
| 49806 | CCNA2 | cyclin A2 | cyclin A2 | 0.84 | 1.10 | 0.24 | 3.30 | 0.83 | 1.13 |
| 49773 | CCNA2 | cyclin A2 | cyclin A2 | 0.87 | 1.02 | 0.25 | 3.51 | 0.81 | 1.08 |
| 49118 | CCNA2 | cyclin A2 | cyclin A2 | 0.75 | 1.10 | 0.23 | 3.12 | 0.84 | 1.00 |
| 49732 | CCND1 | | cyclin D1 (PRAD1: parathyroid adenomatosis | 1.79 | 1.63 | 0.91 | 1.00 | 1.15 | 1.04 |
| | | | 1) | | | | | | |
| 50503 | CCND1 | | cyclin D1 (PRAD1: parathyroid adenomatosis | 1.90 | 1.73 | 0.91 | 1.02 | 1.17 | 1.04 |
| | | | 1) | | | | | | |
| 48015 | CCND3 | Cyclin D3 | cyclin D3 | 0.48 | 2.03 | 0.41 | 2.05 | 0.87 | 1.09 |
| 46608 | CCND3 | Cyclin D3 | cyclin D3 | 0.48 | 1.96 | 0.40 | 2.15 | 0.89 | 1.07 |
| 47926 | CCNE2 | cyclin E2 | cyclin E2 | 1.00 | 1.00 | 0.54 | 1.40 | 0.91 | 1.00 |
| 49967 | CCNE2 | cyclin E2 | cyclin E2 | 0.59 | 1.52 | 0.80 | 1.13 | 0.95 | 1.00 |
| 47927 | CD14 | CD14 | CD14 antigen | 1.61 | 1.40 | 1.65 | 1.13 | 1.23 | 1.00 |
| 49123 | CD38 | CD38 | CD38 antigen (p45) | 1.63 | 1.53 | 0.99 | 1.00 | 0.98 | 1.00 |
| 49252 | CD38 | CD38 | CD38 antigen (p45) | 1.60 | 1.52 | 1.02 | 1.00 | 0.97 | 1.00 |
| 48814 | CDC14A | | CDC14 cell division cycle 14 homolog A (S. cerevisiae) | 0.57 | 1.46 | 1.39 | 1.00 | 1.00 | 1.00 |
| 50229 | CDC2 | | cell division cycle 2, G1 to S and G2 to M | 0.83 | 1.16 | 0.27 | 3.07 | 0.89 | 1.00 |
| 48013 | CDC42EP3 | CEP3 | Cdc42 effector protein 3 | 4.01 | 3.12 | 1.09 | 1.04 | 0.95 | 1.00 |
| 46699 | CDKN1B | p27 | cyclin-dependent kinase inhibitor 1B (p27, | 0.62 | 1.49 | 0.76 | 1.24 | 0.81 | 1.16 |
| | | | Kip1) | | | | | | |
| 49801 | CDKN1B | p27 | cyclin-dependent kinase inhibitor 1B (p27, | 0.48 | 1.91 | 0.68 | 1.42 | 0.85 | 1.06 |
| | | | Kip1) | | | | | | |
| 49815 | CDKN2C | p18 | cyclin-dependent kinase inhibitor 2C (p18, | 1.43 | 1.31 | 0.82 | 1.04 | 1.06 | 1.00 |
| | | | inhibits CDK4) | | | | | | |
| 46842 | CDKN3 | cyclin dependent | cyclin-dependent kinase inhibitor 3 (CDK2- | 0.92 | 1.02 | 0.35 | 2.61 | 1.01 | 1.00 |
| | | kinase inhibitor 3 | associated dual specificity phosphatase) | | | | | | |
| 49105 | CDKN3 | cyclin dependent | cyclin-dependent kinase inhibitor 3 (CDK2- | 0.89 | 1.08 | 0.47 | 1.64 | 1.01 | 1.00 |
| | | kinase inhibitor 3 | associated dual specificity phosphatase) | | | | | | |
| 46849 | CEBPD | CEBP delta | CCAAT/enhancer binding protein (C/EBP), | 1.00 | 1.00 | 0.80 | 1.10 | 0.68 | 1.41 |
| | | | delta | | | | | | |
| 47891 | CEBPD | CEBP delta | CCAAT/enhancer binding protein (C/EBP), | 0.95 | 1.00 | 0.82 | 1.12 | 0.71 | 1.32 |
| | | | delta | | | | | | |
| 48313 | CENPA | | centromere protein A (17 kD) | 0.92 | 1.00 | 0.23 | 4.00 | 0.93 | 1.00 |
| 49737 | CHAF1B | | chromatin assembly factor 1, subunit B (p60) | 0.83 | 1.04 | 0.68 | 1.39 | 1.02 | 1.00 |
| 48672 | CHK | | choline kinase | 1.62 | 1.59 | 1.01 | 1.00 | 0.98 | 1.00 |
| 47032 | CIT | CIT | citron (rho-interacting, serine/threonine kinase | 1.11 | 1.00 | 0.51 | 1.59 | 0.95 | 1.00 |
| | | | 21) | | | | | | |
| 49031 | CKB | | creatine kinase, brain | 2.04 | 1.97 | 0.85 | 1.09 | 0.88 | 1.07 |
| 50590 | CKS2 | | CDC28 protein kinase 2 | 0.92 | 1.06 | 0.53 | 1.69 | 1.00 | 1.00 |
| 47064 | CLGN | | calmegin | 1.02 | 1.00 | 1.69 | 1.61 | 1.03 | 1.00 |
| 50087 | COL4A1 | COL4A1 | collagen, type IV, alpha 1 | 0.58 | 1.55 | 0.96 | 1.00 | 0.94 | 1.00 |
| 48068 | COL4A1 | COL4A1 | collagen, type IV, alpha 1 | 0.61 | 1.44 | 0.96 | 1.00 | 0.99 | 1.00 |
| 49315 | COL4A2 | COL4A2 | collagen, type IV, alpha 2 | 0.63 | 1.44 | 0.91 | 1.01 | 0.85 | 1.02 |
| 47197 | COL4A2 | COL4A2 | collagen, type IV, alpha 2 | 0.63 | 1.33 | 0.89 | 1.01 | 0.88 | 1.00 |
| 48283 | COL5A1 | COL5A1 | collagen, type V, alpha 1 | 0.74 | 1.09 | 1.50 | 1.34 | 0.78 | 1.00 |
| 50125 | COL7A1 | COL7A1 | collagen, type VII, alpha 1 (epidermolysis | 0.54 | 1.60 | 2.14 | 1.91 | 1.05 | 1.00 |
| | | | bullosa, dystrophic, dominant and recessive) | | | | | | |
| 50705 | COL7A1 | COL7A1 | collagen, type VII, alpha 1 (epidermolysis | 0.52 | 1.57 | 2.13 | 1.82 | 0.99 | 1.00 |
| | | | bullosa, dystrophic, dominant and recessive) | | | | | | |
| 49384 | CPS1 | | carbamoyl-phosphate synthetase 1, | 1.29 | 1.22 | 0.67 | 1.36 | 1.01 | 1.00 |
| | | | mitochondrial | | | | | | |
| 48953 | CSDA | CSDA | cold shock domain protein A | 0.69 | 1.36 | 1.02 | 1.00 | 0.84 | 1.17 |
| 47549 | CSDA | CSDA | cold shock domain protein A | 0.64 | 1.36 | 1.06 | 1.00 | 0.86 | 1.09 |
| 48596 | CSF2 | G-CSF, | colony stimulating factor 2 (granulocyte- | 0.20 | 4.11 | 0.72 | 1.14 | 1.01 | 1.00 |
| | | granulocyte colony | macrophage) | | | | | | |
| | | stimulating factor | | | | | | | |
| 48052 | CSF2 | G-CSF, | colony stimulating factor 2 (granulocyte- | 0.20 | 4.31 | 0.63 | 1.09 | 0.92 | 1.00 |
| | | granulocyte colony | macrophage) | | | | | | |
| | | stimulating factor | | | | | | | |
| 50282 | CSRP1 | | cysteine and glycine-rich protein 1 | 0.50 | 1.73 | 1.06 | 1.00 | 1.00 | 1.00 |
| 50462 | CXCL1 | GRO1 | GRO1 oncogene (melanoma growth | 0.15 | 6.10 | 1.24 | 1.16 | 0.94 | 1.00 |
| | | | stimulating activity, alpha) | | | | | | |
| 49754 | CXCL1 | GRO1 | GRO1 oncogene (melanoma growth | 0.16 | 5.65 | 1.15 | 1.00 | 1.18 | 1.00 |
| | | | stimulating activity, alpha) | | | | | | |
| 47558 | CXCL2 | GRO2 | GRO2 oncogene | 0.16 | 4.98 | 0.92 | 1.00 | 0.93 | 1.01 |
| 47632 | CXCL2 | GRO2 | GRO2 oncogene | 0.14 | 5.79 | 1.04 | 1.00 | 0.94 | 1.00 |
| 49692 | CXCL3 | GRO3 | GRO3 oncogene | 0.11 | 8.67 | 0.92 | 1.00 | 0.96 | 1.00 |
| 48330 | CXCL3 | GRO3 | GRO3 oncogene | 0.13 | 7.42 | 0.86 | 1.00 | 0.91 | 1.00 |
| 49839 | CXCL5 | ENA-78 | small inducible cytokine subfamily B (Cys-X- | 0.21 | 4.52 | 1.27 | 1.13 | 0.90 | 1.00 |
| | | | Cys), member 5 (epithelial-derived neutrophil- | | | | | | |
| | | | activating peptide 78) | | | | | | |
| 49968 | CXCL5 | ENA-78 | small inducible cytokine subfamily B (Cys-X- | 0.20 | 4.83 | 1.22 | 1.00 | 0.83 | 1.00 |
| | | | Cys), member 5 (epithelial-derived neutrophil- | | | | | | |
| | | | activating peptide 78) | | | | | | |
| 49752 | CXCR4 | CXCR4 | chemokine (C—X—C motif), receptor 4 (fusin) | 0.40 | 1.70 | 1.08 | 1.00 | 0.96 | 1.00 |
| 49820 | CYC1 | cytochrome c-1 | cytochrome c-1 | 1.47 | 1.34 | 0.79 | 1.12 | 1.13 | 1.03 |
| 47834 | CYP1A1 | | cytochrome P450, subfamily I (aromatic | 1.74 | 1.48 | 1.01 | 1.00 | 0.99 | 1.00 |
| | | | compound-inducible), polypeptide 1 | | | | | | |
| 47412 | CYP24A1 | | cytochrome P450, subfamily XXIV (vitamin D | 0.33 | 2.41 | 0.72 | 1.30 | 1.45 | 1.00 |
| | | | 24-hydroxylase) | | | | | | |
| 50237 | CYR61 | | cysteine-rich, angiogenic inducer, 61 | 1.23 | 1.03 | 1.46 | 1.31 | 0.89 | 1.00 |
| 48650 | DAB2 | | disabled homolog 2, mitogen-responsive | 0.71 | 1.31 | 0.95 | 1.00 | 1.00 | 1.00 |
| | | | phosphoprotein (Drosophila) | | | | | | |
| 46942 | DAF | CD55 | decay accelerating factor for complement | 0.28 | 3.35 | 1.18 | 1.08 | 0.98 | 1.00 |
| | | | (CD55, Cromer blood group system) | | | | | | |
| 48721 | DAF | CD55 | decay accelerating factor for complement | 0.27 | 3.62 | 1.12 | 1.05 | 1.03 | 1.00 |
| | | | (CD55, Cromer blood group system) | | | | | | |
| 48235 | DAXX | Daxx | death-associated protein 6 | 0.58 | 1.52 | 1.01 | 1.00 | 0.96 | 1.00 |
| 49433 | DDIT3 | CHOP | DNA-damage-inducible transcript 3 | 1.25 | 1.06 | 2.05 | 1.86 | 1.08 | 1.00 |
| 50132 | DDIT3 | CHOP | DNA-damage-inducible transcript 3 | 1.18 | 1.04 | 1.88 | 1.64 | 1.01 | 1.00 |
| 48320 | DEFB1 | | defensin, beta 1 | 0.67 | 1.41 | 0.93 | 1.00 | 1.00 | 1.00 |
| 49922 | DHCR24 | | 24-dehydrocholesterol reductase | 1.84 | 1.66 | 0.96 | 1.00 | 0.94 | 1.00 |
| 46700 | DISPB | | KIAA1742 protein | 1.38 | 1.36 | 1.03 | 1.00 | 1.00 | 1.00 |
| 50409 | DNAJB4 | | DnaJ (Hsp40) homolog, subfamily B, member 4 | 0.67 | 1.31 | 0.71 | 1.32 | 0.86 | 1.00 |
| 48333 | DNMT1 | | DNA (cytosine-5-)-methyltransferase 1 | 0.93 | 1.00 | 0.66 | 1.43 | 0.97 | 1.00 |
| 47122 | DSG2 | | desmoglein 2 | 2.17 | 2.05 | 1.15 | 1.12 | 0.99 | 1.00 |
| 50383 | DTR | HB-EGF, diptheria | diphtheria toxin receptor (heparin-binding | 0.43 | 2.17 | 1.02 | 1.00 | 0.89 | 1.00 |
| | | toxin receptor | epidermal growth factor-like growth factor) | | | | | | |
| 50046 | DUSP1 | MKP-1, PTPN10 | dual specificity phosphatase 1 | 2.54 | 1.90 | 0.79 | 1.04 | 0.79 | 1.09 |
| 49389 | DUSP1 | MKP-1, PTPN10 | dual specificity phosphatase 1 | 2.25 | 1.60 | 0.88 | 1.00 | 0.87 | 1.00 |
| 50362 | DUSP10 | DUSP10 | dual specificity phosphatase 10 | 0.24 | 3.05 | 0.85 | 1.08 | 0.86 | 1.04 |
| 48736 | DUSP5 | DUSP5 | dual specificity phosphatase 5 | 1.22 | 1.00 | 1.60 | 1.36 | 1.44 | 1.14 |
| 46670 | DUSP5 | DUSP5 | dual specificity phosphatase 5 | 0.48 | 2.00 | 1.57 | 1.53 | 1.08 | 1.02 |
| 49777 | DUSP6 | DUSP6 | dual specificity phosphatase 6 | 0.46 | 1.88 | 1.38 | 1.32 | 1.03 | 1.00 |
| 49810 | DUSP6 | DUSP6 | dual specificity phosphatase 6 | 0.46 | 1.51 | 1.44 | 1.29 | 1.11 | 1.00 |
| 47277 | DUSP6 | DUSP6 | dual specificity phosphatase 6 | 0.44 | 1.84 | 1.29 | 1.15 | 1.22 | 1.00 |
| 50140 | ECT2 | ECT2 | epithelial cell transforming sequence 2 | 0.61 | 1.53 | 0.53 | 1.47 | 0.82 | 1.14 |
| | | | oncogene | | | | | | |
| 48032 | ECT2 | ECT2 | epithelial cell transforming sequence 2 | 0.47 | 1.96 | 0.41 | 2.16 | 0.84 | 1.10 |
| | | | oncogene | | | | | | |
| 47654 | EFEMP1 | EFEMP1 | EGF-containing fibulin-like extracellular matrix | 0.66 | 1.32 | 0.87 | 1.00 | 1.19 | 1.00 |
| | | | protein 1 | | | | | | |
| 49636 | EGFR | EGFR, ErbB1 | epidermal growth factor receptor (erythroblastic | 0.44 | 1.84 | 1.08 | 1.00 | 0.94 | 1.00 |
| | | | leukemia viral (v-erb-b) oncogene homolog, | | | | | | |
| | | | avian) | | | | | | |
| 50182 | ELA2 | elastase-2 | elastase 2, neutrophil | 1.41 | 1.20 | 1.65 | 1.34 | 1.41 | 1.29 |
| 47811 | ENTPD6 | | ectonucleoside triphosphate | 1.83 | 1.54 | 1.04 | 1.00 | 0.92 | 1.01 |
| | | | diphosphohydrolase 6 (putative function) | | | | | | |
| 49179 | EPAS1 | | endothelial PAS domain protein 1 | 0.65 | 1.44 | 0.80 | 1.16 | 1.02 | 1.00 |
| 49696 | EPOR | erythropoietin R | erythropoietin receptor | 0.40 | 1.72 | 1.04 | 1.00 | 0.96 | 1.00 |
| 47730 | ERBB3 | ErbB3 | v-erb-b2 erythroblastic leukemia viral | 1.83 | 1.30 | 1.80 | 1.73 | 1.30 | 1.12 |
| | | | oncogene homolog 3 (avian) | | | | | | |
| 46599 | ERBB3 | ErbB3 | v-erb-b2 erythroblastic leukemia viral | 2.04 | 1.31 | 1.97 | 1.54 | 1.43 | 1.00 |
| | | | oncogene homolog 3 (avian) | | | | | | |
| 46955 | EREG | epiregulin | epiregulin | 0.11 | 8.09 | 1.19 | 1.14 | 0.98 | 1.00 |
| 50126 | EREG | epiregulin | epiregulin | 0.15 | 4.79 | 1.18 | 1.05 | 1.06 | 1.00 |
| 46988 | EREG | epiregulin | epiregulin | 0.21 | 1.77 | 1.18 | 1.00 | 0.96 | 1.00 |
| 50382 | ETV4 | ETV4 | ets variant gene 4 (E1A enhancer binding | 0.47 | 1.56 | 0.82 | 1.07 | 0.80 | 1.14 |
| | | | protein, E1AF) | | | | | | |
| 49430 | ETV4 | ETV4 | ets variant gene 4 (E1A enhancer binding | 0.57 | 1.46 | 0.83 | 1.09 | 0.86 | 1.06 |
| | | | protein, E1AF) | | | | | | |
| 46984 | ETV5 | ETV5 | ets variant gene 5 (ets-related molecule) | 0.69 | 1.39 | 1.10 | 1.00 | 1.09 | 1.00 |
| 50339 | ETV6 | ETV6 | ets variant gene 6 (TEL oncogene) | 0.61 | 1.30 | 1.19 | 1.09 | 0.95 | 1.00 |
| 48998 | FADS1 | | fatty acid desaturase 1 | 1.20 | 1.09 | 1.57 | 1.38 | 1.04 | 1.00 |
| 49721 | FANCG | | Fanconi anemia, complementation group G | 0.93 | 1.00 | 0.59 | 1.60 | 1.00 | 1.00 |
| 48825 | FARP1 | | FERM, RhoGEF (ARHGEF) and pleckstrin | 1.89 | 1.48 | 0.95 | 1.00 | 0.87 | 1.00 |
| | | | domain protein 1 (chondrocyte-derived) | | | | | | |
| 48350 | FBLN1 | FBLN1 | fibulin 1 | 0.73 | 1.13 | 0.43 | 2.17 | 0.90 | 1.00 |
| 46932 | FBN1 | FBN1 | fibrillin 1 (Marfan syndrome) | 0.60 | 1.38 | 1.13 | 1.00 | 0.86 | 1.10 |
| 50717 | FCER1G | FCER1G | Fc fragment of IgE, high affinity I, receptor for; | 0.64 | 1.34 | 1.04 | 1.00 | 0.84 | 1.00 |
| | | | gamma polypeptide | | | | | | |
| 47748 | FEZ1 | | fasciculation and elongation protein zeta 1 | 0.77 | 1.01 | 1.77 | 1.65 | 1.00 | 1.00 |
| | | | (zygin I) | | | | | | |
| 49529 | FGA | | fibrinogen, A alpha polypeptide | 1.84 | 1.60 | 0.58 | 1.33 | 0.98 | 1.00 |
| 50006 | FGF11 | FGF11 | fibroblast growth factor 11 | 0.96 | 1.00 | 1.79 | 1.66 | 0.79 | 1.00 |
| 49462 | FGF11 | FGF11 | fibroblast growth factor 11 | 1.00 | 1.00 | 1.68 | 1.51 | 0.89 | 1.00 |
| 48520 | FISH | | KIAA0418 gene product | 0.53 | 1.70 | 1.16 | 1.03 | 1.10 | 1.00 |
| 48793 | FLJ10535 | | hypothetical protein FLJ10535 | 1.52 | 1.32 | 0.80 | 1.18 | 1.01 | 1.00 |
| 48328 | FN1 | FN1 | fibronectin 1 | 0.77 | 1.28 | 0.70 | 1.36 | 0.96 | 1.00 |
| 50475 | FOSL1 | FOSL1, Fra1 | FOS-like antigen 1 | 0.47 | 1.94 | 1.45 | 1.40 | 1.06 | 1.00 |
| 50508 | FOSL1 | FOSL1, Fra1 | FOS-like antigen 1 | 0.41 | 2.07 | 1.43 | 1.34 | 1.03 | 1.00 |
| 46993 | FOSL1 | FOSL1, Fra1 | FOS-like antigen 1 | 0.42 | 2.06 | 1.44 | 1.33 | 1.04 | 1.00 |
| 48969 | FOSL2 | FOSL2, Fra2 | FOS-like antigen 2 | 1.77 | 1.71 | 1.34 | 1.13 | 1.13 | 1.02 |
| 48270 | FOSL2 | FOSL2, Fra2 | FOS-like antigen 2 | 1.84 | 1.73 | 1.29 | 1.09 | 1.11 | 1.02 |
| 48219 | FOXM1 | FOXM1 | forkhead box M1 | 0.94 | 1.01 | 0.39 | 2.35 | 0.97 | 1.00 |
| 50681 | FOXM1 | FOXM1 | forkhead box M1 | 0.95 | 1.00 | 0.41 | 1.99 | 0.93 | 1.00 |
| 50701 | FOXO3A | FOXO3A | forkhead box O3A | 2.03 | 1.92 | 1.31 | 1.10 | 0.96 | 1.00 |
| 47923 | FRAP1 | FRAP1, MTOR | FK506 binding protein 12-rapamycin | 1.58 | 1.42 | 1.38 | 1.23 | 1.76 | 1.25 |
| | | | associated protein 1 | | | | | | |
| 48137 | FRDA | | Friedreich ataxia | 0.56 | 1.35 | 0.71 | 1.32 | 1.07 | 1.00 |
| 49883 | FVT1 | | follicular lymphoma variant translocation 1 | 0.75 | 1.31 | 1.12 | 1.08 | 0.97 | 1.00 |
| 46564 | FYB | | FYN binding protein (FYB-120/130) | 1.05 | 1.00 | 1.56 | 1.32 | 1.14 | 1.00 |
| 46824 | FYN | Fyn | FYN oncogene related to SRC, FGR, YES | 0.60 | 1.41 | 0.94 | 1.00 | 1.05 | 1.00 |
| 47526 | FYN | Fyn | FYN oncogene related to SRC, FGR, YES | 0.62 | 1.39 | 1.05 | 1.00 | 0.98 | 1.00 |
| 50580 | G1P2 | ISG15, IFN | interferon-stimulated protein, 15 kDa | 1.51 | 1.14 | 1.95 | 1.60 | 1.63 | 1.38 |
| | | stimulated protein | | | | | | | |
| 46618 | G1P2 | ISG15, IFN | interferon-stimulated protein, 15 kDa | 0.60 | 1.33 | 1.26 | 1.11 | 0.87 | 1.00 |
| | | stimulated protein | | | | | | | |
| 50129 | GAB1 | Gab1 | GRB2-associated binding protein 1 | 0.63 | 1.31 | 1.06 | 1.00 | 1.00 | 1.00 |
| 49675 | GAB2 | GAB2 | GRB2-associated binding protein 2 | 1.73 | 1.60 | 2.04 | 1.58 | 1.94 | 1.65 |
| 49419 | GABPA | GABPA | GA binding protein transcription factor, alpha | 0.58 | 1.53 | 0.88 | 1.10 | 0.95 | 1.00 |
| | | | subunit (60 kD) | | | | | | |
| 48615 | GADD45B | MyD118 | growth arrest and DNA-damage-inducible, beta | 0.54 | 1.50 | 0.40 | 2.30 | 1.00 | 1.00 |
| 50142 | GBP1 | IFN induced GBP-1 | guanylate binding protein 1, interferon- | 0.67 | 1.30 | 1.22 | 1.15 | 0.90 | 1.09 |
| | | | inducible, 67 kD | | | | | | |
| 47432 | GCS1 | | glucosidase I | 1.71 | 1.53 | 0.99 | 1.00 | 0.92 | 1.00 |
| 48651 | GLA | | galactosidase, alpha | 0.51 | 1.89 | 0.87 | 1.11 | 1.02 | 1.00 |
| 48681 | GNB2 | G protein, beta 2 | guanine nucleotide binding protein (G protein), | 0.57 | 1.38 | 1.58 | 1.42 | 0.95 | 1.00 |
| | | | beta polypeptide 2 | | | | | | |
| 48007 | GNB5 | G protein, beta 5 | guanine nucleotide binding protein (G protein), | 0.65 | 1.37 | 0.90 | 1.00 | 1.00 | 1.00 |
| | | | beta 5 | | | | | | |
| 47111 | GOLGB1 | | golgi autoantigen, golgin subfamily b, | 1.03 | 1.00 | 1.43 | 1.32 | 1.05 | 1.00 |
| | | | macrogolgin (with transmembrane signal), 1 | | | | | | |
| 49485 | GPR126 | | hypothetical protein DKFZp564D0462 | 2.48 | 2.31 | 0.55 | 1.63 | 0.97 | 1.00 |
| 48854 | GPR4 | | G protein-coupled receptor 4 | 1.43 | 1.08 | 1.52 | 1.34 | 1.35 | 1.22 |
| 50141 | GRAF | GRAF | GTPase regulator associated with the focal | 1.51 | 1.06 | 1.51 | 1.31 | 1.20 | 1.15 |
| | | | adhesion kinase pp125 | | | | | | |
| 49069 | GRAF | GRAF | GTPase regulator associated with the focal | 2.90 | 2.67 | 0.77 | 1.18 | 0.90 | 1.00 |
| | | | adhesion kinase pp125 | | | | | | |
| 49102 | GRAF | GRAF | GTPase regulator associated with the focal | 2.87 | 2.39 | 0.79 | 1.12 | 0.92 | 1.00 |
| | | | adhesion kinase pp125 | | | | | | |
| 46936 | GRB10 | GRB10 | growth factor receptor-bound protein 10 | 0.41 | 2.26 | 1.71 | 1.63 | 1.18 | 1.11 |
| 46962 | GRB10 | GRB10 | growth factor receptor-bound protein 10 | 0.33 | 2.48 | 1.70 | 1.66 | 1.06 | 1.00 |
| 50018 | GRB2 | Grb2 | growth factor receptor-bound protein 2 | 1.37 | 1.09 | 1.67 | 1.58 | 1.49 | 1.19 |
| 48110 | GREB1 | | KIAA0575 gene product | 1.92 | 1.35 | 2.00 | 1.72 | 1.65 | 1.50 |
| 49586 | GRIN2C | | glutamate receptor, ionotropic, N-methyl D- | 0.14 | 6.71 | 0.72 | 1.11 | 0.96 | 1.00 |
| | | | aspartate 2C | | | | | | |
| 49032 | GSK3B | GSK3B | glycogen synthase kinase 3 beta | 0.69 | 1.31 | 1.19 | 1.02 | 1.05 | 1.00 |
| 49535 | GTF2F2 | | general transcription factor IIF, polypeptide 2 | 0.68 | 1.34 | 0.67 | 1.28 | 0.85 | 1.07 |
| | | | (30 kD subunit) | | | | | | |
| 46792 | HAT1 | | histone acetyltransferase 1 | 0.83 | 1.12 | 0.65 | 1.43 | 0.87 | 1.06 |
| 47502 | HBB | | hemoglobin, beta | 0.70 | 1.33 | 0.92 | 1.02 | 0.98 | 1.00 |
| 50565 | HBP1 | | HMG-box containing protein 1 | 0.64 | 1.45 | 0.94 | 1.00 | 0.99 | 1.00 |
| 47492 | HDGF | | hepatoma-derived growth factor (high-mobility | 0.71 | 1.33 | 0.79 | 1.09 | 0.85 | 1.05 |
| | | | group protein 1-like) | | | | | | |
| 49838 | HLA-DRB1 | MHCII, DR beta 1 | major histocompatibility complex, class II, DR | 0.30 | 2.15 | 1.02 | 1.00 | 0.90 | 1.00 |
| | | | beta 1 | | | | | | |
| 50526 | HMGA1 | HMG-I/Y | high-mobility group (nonhistone chromosomal) | 0.69 | 1.37 | 1.07 | 1.01 | 0.88 | 1.08 |
| | | | protein isoforms I and Y | | | | | | |
| 50493 | HMGA1 | HMG-I/Y | high-mobility group (nonhistone chromosomal) | 0.69 | 1.39 | 1.07 | 1.04 | 0.88 | 1.03 |
| | | | protein isoforms I and Y | | | | | | |
| 50361 | HMGA1 | HMG-I/Y | high-mobility group (nonhistone chromosomal) | 0.70 | 1.35 | 1.09 | 1.02 | 0.90 | 1.02 |
| | | | protein isoforms I and Y | | | | | | |
| 47045 | HMGB1 | | high-mobility group (nonhistone chromosomal) | 0.90 | 1.03 | 0.65 | 1.51 | 0.92 | 1.02 |
| | | | protein 1 | | | | | | |
| 46778 | HMGB2 | | high-mobility group (nonhistone chromosomal) | 0.85 | 1.09 | 0.37 | 2.27 | 0.96 | 1.00 |
| | | | protein 2 | | | | | | |
| 48642 | HPGD | | hydroxyprostaglandin dehydrogenase 15- | 2.55 | 2.26 | 0.55 | 1.39 | 0.83 | 1.14 |
| | | | (NAD) | | | | | | |
| 49113 | HS3ST3A1 | HS3ST3A1 | heparan sulfate (glucosamine) 3-O- | 0.47 | 1.52 | 1.01 | 1.00 | 0.97 | 1.00 |
| | | | sulfotransferase 3A1 | | | | | | |
| 47185 | HSPA1A | HSPA1A | heat shock 70 kD protein 1A | 0.45 | 1.71 | 0.67 | 1.29 | 0.87 | 1.01 |
| 48618 | HSPA1A | HSPA1A | heat shock 70 kD protein 1A | 0.46 | 1.82 | 0.67 | 1.33 | 0.93 | 1.00 |
| 49064 | HSPA1A | HSPA1A | heat shock 70 kD protein 1A | 0.42 | 1.87 | 0.66 | 1.30 | 0.89 | 1.00 |
| 49417 | HSPA1A | HSPA1A | heat shock 70 kD protein 1A | 0.44 | 1.74 | 0.67 | 1.28 | 0.85 | 1.00 |
| 48094 | HSPA6 | HSPA6 | heat shock 70 kD protein 6 (HSP70B′) | 0.51 | 1.52 | 0.71 | 1.22 | 0.90 | 1.00 |
| 49436 | HSPB1 | HSPB1 | heat shock 27 kD protein 1 | 1.90 | 1.62 | 0.86 | 1.07 | 0.87 | 1.06 |
| 48550 | HSPB2 | HSPB2 | heat shock 27 kD protein 2 | 0.41 | 2.30 | 1.21 | 1.16 | 0.97 | 1.00 |
| 48421 | HSPB2 | HSPB2 | heat shock 27 kD protein 2 | 0.41 | 2.14 | 1.19 | 1.12 | 0.95 | 1.00 |
| 48341 | HSPCB | | heat shock 90 kD protein 1, beta | 0.68 | 1.33 | 0.83 | 1.13 | 1.01 | 1.00 |
| 49804 | ICAM1 | ICAM1 | intercellular adhesion molecule 1 (CD54), | 0.74 | 1.00 | 1.72 | 1.50 | 1.11 | 1.08 |
| | | | human rhinovirus receptor | | | | | | |
| 49771 | ICAM1 | ICAM1 | intercellular adhesion molecule 1 (CD54), | 0.75 | 1.00 | 1.71 | 1.54 | 1.10 | 1.06 |
| | | | human rhinovirus receptor | | | | | | |
| 49140 | ICAM1 | ICAM1 | intercellular adhesion molecule 1 (CD54), | 0.77 | 1.00 | 1.71 | 1.57 | 1.11 | 1.05 |
| | | | human rhinovirus receptor | | | | | | |
| 47245 | ID1 | | inhibitor of DNA binding 1, dominant negative | 1.37 | 1.29 | 0.74 | 1.30 | 0.98 | 1.00 |
| | | | helix-loop-helix protein | | | | | | |
| 48913 | IFNAR2 | IFN alpha, beta, | interferon (alpha, beta and omega) receptor 2 | 0.73 | 1.33 | 1.08 | 1.03 | 1.09 | 1.00 |
| | | omega receptor 2 | | | | | | | |
| 49265 | IFNAR2 | IFN alpha, beta, | interferon (alpha, beta and omega) receptor 2 | 0.70 | 1.33 | 1.07 | 1.05 | 1.06 | 1.00 |
| | | omega receptor 2 | | | | | | | |
| 47582 | IFNGR2 | IFN gamma | interferon gamma receptor 2 (interferon | 0.48 | 1.61 | 1.37 | 1.32 | 1.00 | 1.00 |
| | | receptor 2 | gamma transducer 1) | | | | | | |
| 47225 | IFNGR2 | IFN gamma | interferon gamma receptor 2 (interferon | 0.45 | 2.04 | 1.37 | 1.25 | 0.97 | 1.00 |
| | | receptor 2 | gamma transducer 1) | | | | | | |
| 50583 | IGF2 | | insulin-like growth factor 2 (somatomedin A) | 0.69 | 1.00 | 1.76 | 1.59 | 0.90 | 1.00 |
| 50403 | IGFBP3 | | insulin-like growth factor binding protein 3 | 0.15 | 6.13 | 0.90 | 1.04 | 0.94 | 1.02 |
| 48961 | IL10RB | IL-10R beta | interleukin 10 receptor, beta | 1.24 | 1.03 | 1.64 | 1.49 | 1.34 | 1.27 |
| 48597 | IL10RB | IL-10R beta | interleukin 10 receptor, beta | 1.10 | 1.00 | 1.46 | 1.35 | 1.22 | 1.08 |
| 48640 | IL11RA | IL-11R alpha | interleukin 11 receptor, alpha | 1.10 | 1.00 | 1.75 | 1.56 | 0.99 | 1.00 |
| 50013 | IL13RA1 | IL-13Ralpha′ | interleukin 13 receptor, alpha 1 | 1.70 | 1.65 | 0.92 | 1.02 | 1.10 | 1.06 |
| 48581 | IL13RA1 | IL-13Ralpha′ | interleukin 13 receptor, alpha 1 | 1.64 | 1.47 | 0.89 | 1.06 | 1.02 | 1.00 |
| 46970 | IL1B | IL-1, beta | interleukin 1, beta | 0.05 | 17.39 | 2.02 | 1.64 | 1.10 | 1.08 |
| 47656 | IL1B | IL-1, beta | interleukin 1, beta | 0.06 | 3.25 | 2.05 | 1.71 | 1.10 | 1.04 |
| 48637 | IL1R1 | IL-1RI | interleukin 1 receptor, type I | 1.82 | 1.62 | 0.75 | 1.21 | 0.90 | 1.00 |
| 48324 | IL3RA | IL-3R alpha | interleukin 3 receptor, alpha (low affinity) | 1.81 | 1.53 | 2.70 | 2.37 | 2.32 | 1.79 |
| 47925 | IL4R | IL-4R alpha | interleukin 4 receptor | 1.82 | 1.20 | 1.67 | 1.32 | 1.57 | 1.39 |
| 49666 | IL4R | IL-4R alpha | interleukin 4 receptor | 1.50 | 1.28 | 1.65 | 1.55 | 1.58 | 1.22 |
| 49135 | IL6 | interleukin-6, | interleukin 6 (interferon, beta 2) | 0.48 | 1.98 | 0.96 | 1.00 | 0.93 | 1.00 |
| | | IFNbeta2 | | | | | | | |
| 49264 | IL6 | interleukin-6, | interleukin 6 (interferon, beta 2) | 0.53 | 1.60 | 0.92 | 1.00 | 0.95 | 1.00 |
| | | IFNbeta2 | | | | | | | |
| 49289 | IL6R | IL-6R | interleukin 6 receptor | 1.78 | 1.66 | 2.11 | 1.82 | 1.71 | 1.52 |
| 46865 | IL6R | IL-6R | interleukin 6 receptor | 1.76 | 1.63 | 2.00 | 1.67 | 1.73 | 1.48 |
| 46856 | IL8 | interleukin-8 | interleukin 8 | 0.06 | 15.64 | 0.98 | 1.00 | 0.92 | 1.00 |
| 50455 | IMPDH1 | | IMP (inosine monophosphate) dehydrogenase 1 | 1.53 | 1.39 | 1.11 | 1.00 | 0.99 | 1.00 |
| 49873 | INSIG1 | | insulin induced gene 1 | 0.59 | 1.46 | 1.57 | 1.53 | 0.87 | 1.06 |
| 49763 | IQGAP2 | IQGAP2 | IQ motif containing GTPase activating protein 2 | 2.00 | 1.67 | 0.48 | 2.03 | 1.01 | 1.00 |
| 49796 | IQGAP2 | IQGAP2 | IQ motif containing GTPase activating protein 2 | 2.21 | 2.06 | 0.44 | 1.39 | 1.09 | 1.00 |
| 48086 | IQGAP2 | IQGAP2 | IQ motif containing GTPase activating protein 2 | 1.76 | 1.65 | 1.05 | 1.00 | 1.13 | 1.00 |
| 48014 | IRAK1 | IRAK1 | interleukin-1 receptor-associated kinase 1 | 1.57 | 1.43 | 1.55 | 1.50 | 1.20 | 1.10 |
| 47186 | IRAK2 | IRAK-2 | interleukin-1 receptor-associated kinase 2 | 0.54 | 1.53 | 1.36 | 1.05 | 1.03 | 1.00 |
| 49054 | ITGA2 | integrin, alpha 2, | integrin, alpha 2 (CD49B, alpha 2 subunit of | 0.70 | 1.37 | 1.80 | 1.43 | 1.00 | 1.00 |
| | | CD49B, VLA-2 | VLA-2 receptor) | | | | | | |
| 46686 | ITGAV | integrin alpha V, | integrin, alpha V (vitronectin receptor, alpha | 0.66 | 1.37 | 1.15 | 1.06 | 0.96 | 1.00 |
| | | CD51 | polypeptide, antigen CD51) | | | | | | |
| 48127 | ITGB4 | | integrin, beta 4 | 2.81 | 1.60 | 0.85 | 1.04 | 0.91 | 1.00 |
| 46910 | ITPKC | | inositol 1,4,5-trisphosphate 3-kinase C | 1.64 | 1.33 | 1.41 | 1.18 | 1.12 | 1.03 |
| 49518 | ITPR1 | | inositol 1,4,5-triphosphate receptor, type 1 | 0.61 | 1.32 | 0.85 | 1.00 | 1.05 | 1.00 |
| 46820 | JAK1 | Jak1 | Janus kinase 1 (a protein tyrosine kinase) | 1.29 | 1.21 | 1.46 | 1.38 | 1.03 | 1.00 |
| 49322 | JUNB | JunB | jun B proto-oncogene | 0.62 | 1.17 | 0.45 | 1.87 | 0.65 | 1.45 |
| 49319 | JUNB | JunB | jun B proto-oncogene | 0.55 | 1.56 | 0.57 | 1.56 | 0.77 | 1.19 |
| 50528 | KAI1 | CD82 | kangai 1 (suppression of tumorigenicity 6, | 1.52 | 1.23 | 1.59 | 1.42 | 1.44 | 1.15 |
| | | | prostate; CD82 antigen (R2 leukocyte antigen, | | | | | | |
| | | | antigen detected by monoclonal and antibody | | | | | | |
| | | | IA4)) | | | | | | |
| 50495 | KAI1 | CD82 | kangai 1 (suppression of tumorigenicity 6, | 1.44 | 1.17 | 1.68 | 1.44 | 1.33 | 1.00 |
| | | | prostate; CD82 antigen (R2 leukocyte antigen, | | | | | | |
| | | | antigen detected by monoclonal and antibody | | | | | | |
| | | | IA4)) | | | | | | |
| 48978 | KBRAS1 | KBRAS1 | I-kappa-B-interacting Ras-like protein 1 | 0.59 | 1.44 | 0.93 | 1.00 | 1.02 | 1.00 |
| 49805 | KBRAS2 | KBRAS2 | I-kappa-B-interacting Ras-like protein 2 | 1.46 | 1.28 | 1.74 | 1.46 | 1.41 | 1.25 |
| 49109 | KBRAS2 | KBRAS2 | I-kappa-B-interacting Ras-like protein 2 | 1.43 | 1.06 | 1.78 | 1.46 | 1.40 | 1.21 |
| 49235 | KCNK1 | | potassium channel, subfamily K, member 1 | 0.61 | 1.51 | 0.97 | 1.00 | 0.97 | 1.00 |
| | | | (TWIK-1) | | | | | | |
| 49239 | KCNMA1 | | potassium large conductance calcium- | 1.07 | 1.00 | 1.53 | 1.39 | 1.13 | 1.00 |
| | | | activated channel, subfamily M, alpha member 1 | | | | | | |
| 47770 | KIAA0062 | | KIAA0062 protein | 2.97 | 2.84 | 1.06 | 1.00 | 0.99 | 1.00 |
| 48663 | KIAA0089 | | KIAA0089 protein | 1.64 | 1.52 | 1.14 | 1.12 | 1.01 | 1.00 |
| 50636 | KIAA0101 | | KIAA0101 gene product | 0.89 | 1.00 | 0.34 | 2.41 | 0.94 | 1.00 |
| 48103 | KIAA0205 | | KIAA0205 gene product | 0.71 | 1.34 | 0.85 | 1.15 | 1.03 | 1.00 |
| 48081 | KIAA0220 | | KIAA0220 protein | 1.59 | 1.38 | 1.47 | 1.31 | 1.54 | 1.50 |
| 48918 | KIAA0514 | | KIAA0514 gene product | 1.41 | 1.35 | 1.07 | 1.02 | 1.07 | 1.00 |
| 50582 | KIAA0534 | | KIAA0534 protein | 1.73 | 1.45 | 1.03 | 1.00 | 0.89 | 1.00 |
| 50283 | KIAA1223 | | KIAA1223 protein | 0.60 | 1.54 | 0.96 | 1.00 | 0.94 | 1.00 |
| 50568 | KIF23 | | kinesin-like 5 (mitotic kinesin-like protein 1) | 1.02 | 1.00 | 0.51 | 1.82 | 0.94 | 1.00 |
| 48479 | KIF3C | | kinesin family member 3C | 0.57 | 1.37 | 0.96 | 1.00 | 1.02 | 1.00 |
| 48162 | KIFC1 | | kinesin-like 2 | 1.07 | 1.00 | 0.37 | 2.25 | 1.03 | 1.00 |
| 48990 | KRAS2 | K-Ras | v-Ki-ras2 Kirsten rat sarcoma 2 viral oncogene | 1.48 | 1.39 | 0.84 | 1.11 | 1.01 | 1.00 |
| | | | homolog | | | | | | |
| 48856 | KRT2A | | keratin 2A (epidermal ichthyosis bullosa of | 2.49 | 1.71 | 0.81 | 1.06 | 0.86 | 1.00 |
| | | | Siemens) | | | | | | |
| 46833 | KSR | KSR1 | kinase suppressor of ras | 2.00 | 1.36 | 0.90 | 1.02 | 1.34 | 1.00 |
| 48225 | KSR | KSR1 | kinase suppressor of ras | 1.67 | 1.31 | 0.97 | 1.00 | 1.05 | 1.00 |
| 48621 | LAMB1 | LAMB1 | laminin, beta 1 | 1.81 | 1.56 | 0.83 | 1.12 | 0.99 | 1.00 |
| 50042 | LAMB3 | LAMB3 | laminin, beta 3 (nicein (125 kD), kalinin | 0.30 | 2.95 | 0.94 | 1.00 | 0.85 | 1.04 |
| | | | (140 kD), BM600 (125 kD)) | | | | | | |
| 48692 | LAMC1 | LAMC1 | laminin, gamma 1 (formerly LAMB2) | 0.84 | 1.05 | 0.55 | 1.76 | 1.09 | 1.00 |
| 50393 | LAMC2 | LAMC2 | laminin, gamma 2 (nicein (100 kD), kalinin | 0.31 | 2.63 | 1.65 | 1.48 | 0.99 | 1.00 |
| | | | (105 kD), BM600 (100 kD), Herlitz junctional | | | | | | |
| | | | epidermolysis bullosa)) | | | | | | |
| 50037 | LAMC2 | LAMC2 | laminin, gamma 2 (nicein (100 kD), kalinin | 0.19 | 4.28 | 1.75 | 1.46 | 1.05 | 1.00 |
| | | | (105 kD), BM600 (100 kD), Herlitz junctional | | | | | | |
| | | | epidermolysis bullosa)) | | | | | | |
| 49966 | LBP | LPS binding | lipopolysaccharide binding protein | 1.87 | 1.66 | 0.78 | 1.24 | 0.91 | 1.05 |
| | | protein | | | | | | | |
| 49837 | LBP | LPS binding | lipopolysaccharide binding protein | 1.99 | 1.69 | 0.80 | 1.19 | 0.99 | 1.00 |
| | | protein | | | | | | | |
| 46939 | LDHA | | lactate dehydrogenase A | 1.55 | 1.48 | 0.91 | 1.06 | 1.01 | 1.00 |
| 49683 | LEPR | leptin R | leptin receptor | 1.92 | 1.67 | 1.89 | 1.54 | 1.75 | 1.72 |
| 49163 | LGALS3 | | lectin, galactoside-binding, soluble, 3 (galectin | 1.05 | 1.00 | 1.37 | 1.32 | 1.09 | 1.04 |
| | | | 3) | | | | | | |
| 47482 | LGALS4 | | lectin, galactoside-binding, soluble, 4 (galectin | 1.25 | 1.03 | 1.58 | 1.35 | 1.05 | 1.00 |
| | | | 4) | | | | | | |
| 47191 | LIFR | LIFR | leukemia inhibitory factor receptor | 1.51 | 1.24 | 1.54 | 1.41 | 1.05 | 1.00 |
| 50025 | LIFR | LIFR | leukemia inhibitory factor receptor | 1.55 | 1.32 | 1.41 | 1.22 | 0.97 | 1.00 |
| 47114 | LPP | | LIM domain containing preferred translocation | 1.61 | 1.39 | 1.02 | 1.00 | 0.97 | 1.00 |
| | | | partner in lipoma | | | | | | |
| 48105 | LTBP1 | | latent transforming growth factor beta binding | 1.63 | 1.56 | 0.86 | 1.09 | 0.99 | 1.00 |
| | | | protein 1 | | | | | | |
| 47533 | LTBP4 | LTBP4 | latent transforming growth factor beta binding | 0.68 | 1.34 | 1.36 | 1.04 | 0.82 | 1.11 |
| | | | protein 4 | | | | | | |
| 46676 | LYN | Lyn | v-yes-1 Yamaguchi sarcoma viral related | 0.65 | 1.37 | 0.96 | 1.00 | 0.94 | 1.00 |
| | | | oncogene homolog | | | | | | |
| 49376 | MAC30 | | hypothetical protein | 1.00 | 1.00 | 0.64 | 1.49 | 1.07 | 1.00 |
| 47029 | MAD | MAD | MAX dimerization protein | 0.65 | 1.40 | 1.57 | 1.51 | 0.87 | 1.09 |
| 47158 | MAD | MAD | MAX dimerization protein | 0.61 | 1.36 | 1.58 | 1.52 | 0.90 | 1.00 |
| 46776 | MAD2L1 | | MAD2 mitotic arrest deficient-like 1 (yeast) | 0.98 | 1.00 | 0.43 | 2.18 | 1.07 | 1.00 |
| 46705 | MAOA | | monoamine oxidase A | 2.63 | 1.77 | 1.01 | 1.00 | 0.84 | 1.00 |
| 48150 | MAP1B | | microtubule-associated protein 1B | 0.68 | 1.29 | 1.89 | 1.73 | 1.17 | 1.00 |
| 48278 | MAP2K3 | MKK3 | mitogen-activated protein kinase kinase 3 | 0.51 | 1.83 | 0.98 | 1.00 | 0.83 | 1.10 |
| 48028 | MAP2K3 | MKK3 | mitogen-activated protein kinase kinase 3 | 0.63 | 1.51 | 1.07 | 1.00 | 0.89 | 1.07 |
| 47299 | MAP3K10 | MLK2 | mitogen-activated protein kinase kinase kinase | 1.46 | 1.00 | 1.77 | 1.60 | 1.19 | 1.05 |
| | | | 10 | | | | | | |
| 47561 | MAP3K11 | MLK3 | mitogen-activated protein kinase kinase kinase | 0.58 | 1.46 | 0.65 | 1.47 | 0.74 | 1.12 |
| | | | 11 | | | | | | |
| 47218 | MAP3K7IP1 | TAB1 | transforming growth factor beta-activated | 2.04 | 1.61 | 1.93 | 1.50 | 1.88 | 1.70 |
| | | | kinase-binding protein 1 | | | | | | |
| 47001 | MAP3K7IP1 | TAB1 | transforming growth factor beta-activated | 1.80 | 1.50 | 2.05 | 1.65 | 1.53 | 1.06 |
| | | | kinase-binding protein 1 | | | | | | |
| 47969 | MAP3K8 | c-cot | mitogen-activated protein kinase kinase kinase 8 | 0.25 | 2.86 | 0.85 | 1.14 | 0.86 | 1.00 |
| 48424 | MAP4K4 | GCK-like, HPK, | mitogen-activated protein kinase kinase kinase | 0.57 | 1.48 | 1.49 | 1.45 | 1.05 | 1.02 |
| | | HGK, NIK | kinase 4 | | | | | | |
| 46601 | MAPK14 | p38, alpha | mitogen-activated protein kinase 14 | 1.48 | 1.39 | 1.27 | 1.00 | 1.04 | 1.00 |
| 48410 | MAPK6 | MAPK6 | mitogen-activated protein kinase 6 | 0.52 | 1.86 | 1.07 | 1.00 | 1.01 | 1.00 |
| 48377 | MAPK6 | MAPK6 | mitogen-activated protein kinase 6 | 0.52 | 1.82 | 0.98 | 1.00 | 0.99 | 1.00 |
| 46998 | MAPK6 | MAPK6 | mitogen-activated protein kinase 6 | 0.55 | 1.71 | 0.96 | 1.00 | 1.07 | 1.00 |
| 46965 | MAPK6 | MAPK6 | mitogen-activated protein kinase 6 | 0.56 | 1.69 | 1.00 | 1.00 | 1.03 | 1.00 |
| 47188 | MAPK8 | JNK1 | mitogen-activated protein kinase 8 | 0.69 | 1.36 | 0.99 | 1.00 | 1.04 | 1.00 |
| 49304 | MAPK8IP3 | MAPK8IP3 | mitogen-activated protein kinase 8 interacting | 1.06 | 1.00 | 1.47 | 1.31 | 0.94 | 1.00 |
| | | | protein 3 | | | | | | |
| 48760 | MAPK8IP3 | MAPK8IP3 | mitogen-activated protein kinase 8 interacting | 1.05 | 1.00 | 1.57 | 1.31 | 0.85 | 1.00 |
| | | | protein 3 | | | | | | |
| 48108 | MAT2A | | methionine adenosyltransferase II, alpha | 1.50 | 1.35 | 0.93 | 1.00 | 0.93 | 1.00 |
| 49511 | MATN2 | | matrilin 2 | 1.11 | 1.02 | 0.73 | 1.33 | 1.06 | 1.00 |
| 50566 | MCC | | mutated in colorectal cancers | 1.64 | 1.06 | 1.80 | 1.43 | 1.26 | 1.00 |
| 47123 | MCM2 | | MCM2 minichromosome maintenance deficient | 0.80 | 1.09 | 0.52 | 1.66 | 0.84 | 1.14 |
| | | | 2, mitotin (S. cerevisiae) | | | | | | |
| 48470 | MCM5 | | MCM5 minichromosome maintenance deficient | 0.79 | 1.10 | 0.43 | 1.99 | 0.93 | 1.00 |
| | | | 5, cell division cycle 46 (S. cerevisiae) | | | | | | |
| 48593 | MEF2D | MEF2D | MADS box transcription enhancer factor 2, | 0.72 | 1.35 | 0.90 | 1.06 | 0.93 | 1.00 |
| | | | polypeptide D (myocyte enhancer factor 2D) | | | | | | |
| 46692 | MID1 | | midline 1 (Opitz/BBB syndrome) | 0.86 | 1.10 | 0.62 | 1.35 | 0.96 | 1.00 |
| 46610 | MIG-6 | MIG-6 | Gene 33/Mig-6 | 2.34 | 2.24 | 0.88 | 1.02 | 1.04 | 1.01 |
| 48286 | MIG-6 | MIG-6 | Gene 33/Mig-6 | 2.26 | 1.85 | 0.87 | 1.00 | 1.08 | 1.00 |
| 47385 | MLAT4 | | hypothetical protein FLJ10718 | 2.24 | 2.08 | 0.78 | 1.14 | 1.12 | 1.03 |
| 48966 | MMP10 | MMP10 | matrix metalloproteinase 10 (stromelysin 2) | 1.00 | 1.00 | 2.33 | 1.94 | 0.84 | 1.00 |
| 48003 | MMP19 | MMP19 | matrix metalloproteinase 19 | 1.61 | 1.31 | 2.17 | 2.04 | 1.40 | 1.33 |
| 48933 | MMP2 | MMP2, gelatinase A | matrix metalloproteinase 2 (gelatinase A, 72 kD | 0.64 | 1.35 | 0.98 | 1.00 | 0.85 | 1.04 |
| | | | gelatinase, 72 kD type IV collagenase) | | | | | | |
| 48334 | MMP7 | MMP7 | matrix metalloproteinase 7 (matrilysin, uterine) | 0.32 | 2.44 | 0.55 | 1.55 | 1.07 | 1.00 |
| 49124 | MMP7 | MMP7 | matrix metalloproteinase 7 (matrilysin, uterine) | 0.43 | 2.23 | 0.66 | 1.39 | 1.04 | 1.00 |
| 49115 | MMPL1 | MMPL1 | matrix metalloproteinase-like 1 | 2.08 | 1.12 | 2.17 | 1.90 | 1.69 | 1.49 |
| 50034 | MMPL1 | MMPL1 | matrix metalloproteinase-like 1 | 2.49 | 1.81 | 2.46 | 2.22 | 1.30 | 1.00 |
| 48342 | MMPL1 | MMPL1 | matrix metalloproteinase-like 1 | 1.00 | 1.00 | 2.07 | 1.54 | 1.34 | 1.00 |
| 49259 | MOX2 | MOX2 | antigen identified by monoclonal antibody MRC | 1.75 | 1.59 | 0.87 | 1.02 | 0.98 | 1.00 |
| | | | OX-2 | | | | | | |
| 47501 | MOX2 | MOX2 | antigen identified by monoclonal antibody MRC | 1.70 | 1.54 | 0.92 | 1.00 | 0.96 | 1.00 |
| | | | OX-2 | | | | | | |
| 50770 | MPHOSPH1 | | M-phase phosphoprotein 1 | 1.03 | 1.00 | 0.59 | 1.37 | 0.97 | 1.00 |
| 48187 | MRPL27 | | mitochondrial ribosomal protein L27 | 1.08 | 1.00 | 0.66 | 1.40 | 1.01 | 1.00 |
| 49860 | MSX1 | | msh homeo box homolog 1 (Drosophila) | 0.54 | 1.63 | 0.96 | 1.00 | 0.85 | 1.09 |
| 47106 | MT1L | | metallothionein 1L | 6.96 | 5.74 | 1.29 | 1.08 | 0.89 | 1.06 |
| 46766 | MT3 | | metallothionein 3 (growth inhibitory factor | 2.31 | 2.21 | 1.22 | 1.15 | 0.89 | 1.00 |
| | | | (neurotrophic)) | | | | | | |
| 50240 | MTIF2 | | mitochondrial translational initiation factor 2 | 1.52 | 1.43 | 0.96 | 1.00 | 0.93 | 1.04 |
| 48399 | MUC1 | mucin1 | mucin 1, transmembrane | 2.06 | 1.62 | 0.93 | 1.00 | 0.84 | 1.00 |
| 48735 | MUC13 | mucin13 | mucin 13, epithelial transmembrane | 0.49 | 1.50 | 0.66 | 1.29 | 0.83 | 1.03 |
| 50390 | MUC13 | mucin13 | mucin 13, epithelial transmembrane | 0.47 | 1.41 | 0.76 | 1.13 | 0.97 | 1.00 |
| 47267 | MXD4 | MAD4 | Mad4 homolog | 1.15 | 1.00 | 1.52 | 1.36 | 0.77 | 1.00 |
| 47640 | NaGLT1 | | KIAA1919 protein | 1.72 | 1.24 | 1.76 | 1.49 | 1.33 | 1.27 |
| 47562 | NaGLT1 | | KIAA1919 protein | 1.71 | 1.34 | 1.27 | 1.15 | 1.18 | 1.09 |
| 48487 | NDN | | necdin homolog (mouse) | 1.66 | 1.26 | 1.70 | 1.57 | 1.31 | 1.18 |
| 46799 | NFE2L1 | NFE2L1 | nuclear factor (erythroid-derived 2)-like 1 | 0.69 | 1.34 | 1.13 | 1.00 | 0.97 | 1.00 |
| 47173 | NFKB1 | NFkappaB, p105 | nuclear factor of kappa light polypeptide gene | 0.39 | 2.20 | 0.84 | 1.06 | 0.89 | 1.10 |
| | | | enhancer in B-cells 1 (p105) | | | | | | |
| 47877 | NFKB1 | NFkappaB, p105 | nuclear factor of kappa light polypeptide gene | 0.40 | 2.13 | 0.95 | 1.00 | 0.89 | 1.04 |
| | | | enhancer in B-cells 1 (p105) | | | | | | |
| 49690 | NID | NID | nidogen (enactin) | 0.72 | 1.00 | 0.68 | 1.34 | 0.87 | 1.03 |
| 48967 | NID2 | NID2 | nidogen 2 | 0.93 | 1.00 | 1.85 | 1.72 | 1.05 | 1.00 |
| 50131 | NID2 | NID2 | nidogen 2 | 1.43 | 1.21 | 1.64 | 1.36 | 1.03 | 1.00 |
| 49014 | NINJ1 | | ninjurin 1 | 0.53 | 1.78 | 1.02 | 1.00 | 0.93 | 1.04 |
| 48029 | NOL3 | NOL3 | nucleolar protein 3 (apoptosis repressor with | 1.79 | 1.62 | 1.01 | 1.00 | 0.84 | 1.03 |
| | | | CARD domain) | | | | | | |
| 49158 | NOS1 | | nitric oxide synthase 1 (neuronal) | 1.46 | 1.28 | 1.79 | 1.30 | 1.48 | 1.17 |
| 48941 | NR4A1 | NAK1 | nuclear receptor subfamily 4, group A, member 1 | 0.34 | 2.72 | 0.54 | 1.61 | 0.87 | 1.09 |
| 49383 | NR4A1 | NAK1 | nuclear receptor subfamily 4, group A, member 1 | 0.54 | 1.55 | 0.88 | 1.01 | 0.96 | 1.00 |
| 47712 | NR4A2 | TINUR | nuclear receptor subfamily 4, group A, member 2 | 0.29 | 1.66 | 0.89 | 1.00 | 0.85 | 1.12 |
| 47679 | NR4A2 | TINUR | nuclear receptor subfamily 4, group A, member 2 | 0.33 | 1.48 | 0.93 | 1.02 | 0.88 | 1.02 |
| 48227 | NRG1 | neuregulin1, neu | neuregulin 1 | 0.25 | 3.29 | 0.81 | 1.17 | 0.99 | 1.00 |
| | | diff. Factor, | | | | | | | |
| | | heregulin | | | | | | | |
| 48468 | NRG1 | neuregulin1, neu | neuregulin 1 | 0.40 | 2.31 | 1.03 | 1.00 | 1.01 | 1.00 |
| | | diff. Factor, | | | | | | | |
| | | heregulin | | | | | | | |
| 47416 | NTS | | neurotensin | 1.05 | 1.00 | 0.64 | 1.41 | 1.04 | 1.00 |
| 47183 | OSMR | oncostatin M R | oncostatin M receptor | 0.55 | 1.80 | 1.07 | 1.03 | 0.99 | 1.00 |
| 49778 | OSMR | oncostatin M R | oncostatin M receptor | 0.59 | 1.60 | 1.11 | 1.00 | 0.93 | 1.00 |
| 49585 | OSRF | | osmosis responsive factor | 0.55 | 1.43 | 0.92 | 1.00 | 1.02 | 1.00 |
| 47860 | P2RXL1 | P2RXL1 | purinergic receptor P2X-like 1, orphan receptor | 1.59 | 1.40 | 0.89 | 1.00 | 0.97 | 1.00 |
| 48193 | P2RY1 | P2RY1 | purinergic receptor P2Y, G-protein coupled, 1 | 0.62 | 1.49 | 0.89 | 1.04 | 1.14 | 1.00 |
| 47358 | P2RY2 | P2RY2 | purinergic receptor P2Y, G-protein coupled, 2 | 0.84 | 1.02 | 2.23 | 1.87 | 1.13 | 1.00 |
| 47902 | P2RY2 | P2RY2 | purinergic receptor P2Y, G-protein coupled, 2 | 1.00 | 1.00 | 2.06 | 1.54 | 0.90 | 1.00 |
| 48707 | P4HA1 | P4HA1, prolyl-4- | procollagen-proline, 2-oxoglutarate 4- | 0.52 | 1.58 | 0.88 | 1.00 | 0.89 | 1.06 |
| | | hydroxylase | dioxygenase (proline 4-hydroxylase), alpha | | | | | | |
| | | alpha1 | polypeptide I | | | | | | |
| 47935 | P4HA1 | P4HA1, prolyl-4- | procollagen-proline, 2-oxoglutarate 4- | 0.54 | 1.72 | 1.03 | 1.00 | 0.96 | 1.00 |
| | | hydroxylase | dioxygenase (proline 4-hydroxylase), alpha | | | | | | |
| | | alpha1 | polypeptide I | | | | | | |
| 49905 | PAX3 | | paired box gene 3 (Waardenburg syndrome 1) | 0.49 | 1.58 | 1.11 | 1.03 | 0.96 | 1.00 |
| 49707 | PCNA | | proliferating cell nuclear antigen | 0.79 | 1.13 | 0.41 | 1.60 | 1.08 | 1.00 |
| 49705 | PCYT2 | | phosphate cytidylyltransferase 2, ethanolamine | 1.66 | 1.42 | 1.39 | 1.24 | 1.00 | 1.00 |
| 49933 | PDE3A | | phosphodiesterase 3A, cGMP-inhibited | 0.76 | 1.00 | 0.65 | 1.42 | 1.05 | 1.00 |
| 48556 | PDE4B | phosphodiesterase | phosphodiesterase 4B, cAMP-specific | 0.38 | 2.21 | 1.35 | 1.20 | 1.22 | 1.13 |
| | | 4B | (phosphodiesterase E4 dunce homolog, | | | | | | |
| | | | Drosophila) | | | | | | |
| 48427 | PDE4B | phosphodiesterase | phosphodiesterase 4B, cAMP-specific | 0.40 | 2.15 | 1.41 | 1.27 | 1.13 | 1.00 |
| | | 4B | (phosphodiesterase E4 dunce homolog, | | | | | | |
| | | | Drosophila) | | | | | | |
| 48370 | PDGFA | PDGF, alpha | platelet-derived growth factor alpha | 0.82 | 1.03 | 1.89 | 1.63 | 1.17 | 1.00 |
| | | | polypeptide | | | | | | |
| 50639 | PDIR | | for protein disulfide isomerase-related | 1.62 | 1.39 | 1.20 | 1.08 | 1.04 | 1.00 |
| 49965 | PF4 | platelet factor 4 | platelet factor 4 | 0.66 | 1.39 | 0.95 | 1.00 | 1.04 | 1.00 |
| 47639 | PGAM1 | | phosphoglycerate mutase 1 (brain) | 1.60 | 1.42 | 0.87 | 1.08 | 1.13 | 1.00 |
| 46890 | PHKA1 | | phosphorylase kinase, alpha 1 (muscle) | 1.59 | 1.39 | 1.01 | 1.00 | 0.96 | 1.02 |
| 47597 | PHKG2 | | phosphorylase kinase, gamma 2 (testis) | 1.53 | 1.49 | 1.01 | 1.00 | 0.97 | 1.00 |
| 49854 | PI3 | PI3 | protease inhibitor 3, skin-derived (SKALP) | 0.34 | 2.41 | 1.23 | 1.12 | 1.06 | 1.00 |
| 47320 | PI3 | PI3 | protease inhibitor 3, skin-derived (SKALP) | 0.33 | 2.69 | 1.15 | 1.06 | 1.13 | 1.00 |
| 47151 | PIK3C3 | PI3K class 3 | phosphoinositide-3-kinase, class 3 | 0.64 | 1.32 | 1.12 | 1.00 | 1.09 | 1.02 |
| 46818 | PIM2 | Pim-2 | pim-2 oncogene | 0.56 | 1.65 | 1.12 | 1.00 | 0.73 | 1.11 |
| 48195 | PLA2G1B | phospholipase A2 | phospholipase A2, group IB (pancreas) | 1.74 | 1.41 | 1.67 | 1.58 | 1.76 | 1.46 |
| 47118 | PLA2G4A | phospholipase A2 | phospholipase A2, group IVA (cytosolic, | 0.49 | 1.90 | 0.76 | 1.27 | 0.97 | 1.00 |
| | | | calcium-dependent) | | | | | | |
| 48023 | PLAB | PLAB | prostate differentiation factor | 0.08 | 9.91 | 0.72 | 1.18 | 0.75 | 1.17 |
| 50434 | PLAB | PLAB | prostate differentiation factor | 0.08 | 11.20 | 0.75 | 1.13 | 0.72 | 1.13 |
| 47541 | PLAT | plasminogen | plasminogen activator, tissue | 1.00 | 1.00 | 1.41 | 1.30 | 1.18 | 1.00 |
| | | activator | | | | | | | |
| 47537 | PLAU | urokinase-type | plasminogen activator, urokinase | 0.41 | 2.23 | 0.98 | 1.00 | 1.14 | 1.05 |
| | | plasminogen | | | | | | | |
| | | activator | | | | | | | |
| 49424 | PLAU | urokinase-type | plasminogen activator, urokinase | 0.42 | 2.26 | 0.78 | 1.02 | 1.08 | 1.00 |
| | | plasminogen | | | | | | | |
| | | activator | | | | | | | |
| 48138 | PLAUR | | plasminogen activator, urokinase receptor | 0.28 | 3.27 | 1.04 | 1.00 | 0.94 | 1.00 |
| 49106 | PLOD | PLOD | procollagen-lysine, 2-oxoglutarate 5- | 0.83 | 1.00 | 0.70 | 1.37 | 0.93 | 1.00 |
| | | | dioxygenase (lysine hydroxylase, Ehlers- | | | | | | |
| | | | Danlos syndrome type VI) | | | | | | |
| 49073 | PLOD | PLOD | procollagen-lysine, 2-oxoglutarate 5- | 0.87 | 1.03 | 0.72 | 1.35 | 0.95 | 1.00 |
| | | | dioxygenase (lysine hydroxylase, Ehlers- | | | | | | |
| | | | Danlos syndrome type VI) | | | | | | |
| 50313 | PLOD2 | PLOD2 | procollagen-lysine, 2-oxoglutarate 5- | 0.39 | 2.39 | 1.09 | 1.00 | 0.89 | 1.05 |
| | | | dioxygenase (lysine hydroxylase) 2 | | | | | | |
| 49325 | PLOD2 | PLOD2 | procollagen-lysine, 2-oxoglutarate 5- | 0.33 | 2.87 | 1.15 | 1.00 | 0.97 | 1.00 |
| | | | dioxygenase (lysine hydroxylase) 2 | | | | | | |
| 47776 | POLA | | polymerase (DNA directed), alpha | 0.87 | 1.04 | 0.57 | 1.63 | 0.92 | 1.00 |
| 46908 | POLR2J | | polymerase (RNA) II (DNA directed) | 0.56 | 1.45 | 1.12 | 1.08 | 1.00 | 1.00 |
| | | | polypeptide J (13.3 kD) | | | | | | |
| 49950 | PPAP2B | | phosphatidic acid phosphalase type 2B | 0.63 | 1.48 | 1.03 | 1.00 | 0.88 | 1.06 |
| 47328 | PPFIBP1 | PPFIBP1 | PTPRF interacting protein, binding protein 1 | 0.50 | 1.82 | 1.23 | 1.10 | 1.07 | 1.00 |
| | | | (liprin beta 1) | | | | | | |
| 49413 | PPID | cyclophilin D | peptidylprolyl isomerase D (cyclophilin D) | 1.46 | 1.33 | 1.81 | 1.56 | 1.65 | 1.42 |
| 49103 | PPID | cyclophilin D | peptidylprolyl isomerase D (cyclophilin D) | 1.66 | 1.27 | 1.98 | 1.74 | 1.59 | 1.41 |
| 48616 | PPIH | cyclophilin H | peptidyl prolyl isomerase H (cyclophilin H) | 1.18 | 1.14 | 0.65 | 1.39 | 1.03 | 1.00 |
| 48726 | PPP2R2B | PP2 regulatory | protein phosphatase 2 (formerly 2A), regulatory | 1.45 | 1.12 | 1.74 | 1.56 | 1.53 | 1.18 |
| | | subunit B beta | subunit B (PR 52), beta isoform | | | | | | |
| 46994 | PPP2R2B | PP2 regulatory | protein phosphatase 2 (formerly 2A), regulatory | 1.34 | 1.00 | 1.80 | 1.66 | 1.39 | 1.00 |
| | | subunit B beta | subunit B (PR 52), beta isoform | | | | | | |
| 46961 | PPP2R2B | PP2 regulatory | protein phosphatase 2 (formerly 2A), regulatory | 1.55 | 1.00 | 1.73 | 1.64 | 1.15 | 1.00 |
| | | subunit B beta | subunit B (PR 52), beta isoform | | | | | | |
| 47779 | PRDM2 | | PR domain containing 2, with ZNF domain | 1.30 | 1.09 | 1.47 | 1.42 | 1.00 | 1.00 |
| 48231 | PRKACG | PKA, catalytic | protein kinase, cAMP-dependent, catalytic, | 0.64 | 1.46 | 0.73 | 1.27 | 0.92 | 1.00 |
| | | subunit gamma | gamma | | | | | | |
| 46973 | PRKAR1B | PKA, regulatory | protein kinase, cAMP-dependent, regulatory, | 1.66 | 1.36 | 1.82 | 1.52 | 1.39 | 1.06 |
| | | subunit, type I beta | type I, beta | | | | | | |
| 47006 | PRKAR1B | PKA, regulatory | protein kinase, cAMP-dependent, regulatory, | 1.52 | 1.46 | 1.79 | 1.42 | 1.32 | 1.00 |
| | | subunit, type I beta | type I, beta | | | | | | |
| 49738 | PRKCA | PKC alpha | protein kinase C, alpha | 1.19 | 1.02 | 1.76 | 1.43 | 1.38 | 1.26 |
| 46934 | PRKCD | PKC, delta | protein kinase C, delta | 1.91 | 1.35 | 0.98 | 1.00 | 0.88 | 1.09 |
| 47528 | PRKCN | PKC, nu | protein kinase C, nu | 0.68 | 1.45 | 1.14 | 1.08 | 1.01 | 1.00 |
| 47176 | PTGES | PTGES | prostaglandin E synthase | 0.61 | 1.55 | 1.07 | 1.00 | 1.22 | 1.10 |
| 46632 | PTGES | PTGES | prostaglandin E synthase | 0.63 | 1.43 | 1.00 | 1.00 | 1.17 | 1.08 |
| 47499 | PTGS2 | PTGS2, COX2 | prostaglandin-endoperoxide synthase 2 | 0.05 | 18.58 | 0.99 | 1.00 | 0.88 | 1.00 |
| | | | (prostaglandin G/H synthase and | | | | | | |
| | | | cyclooxygenase) | | | | | | |
| 50665 | PTGS2 | PTGS2, COX2 | prostaglandin-endoperoxide synthase 2 | 0.06 | 17.96 | 0.96 | 1.00 | 0.87 | 1.00 |
| | | | (prostaglandin G/H synthase and | | | | | | |
| | | | cyclooxygenase) | | | | | | |
| 49036 | PTK2B | PYK2 | protein tyrosine kinase 2 beta | 1.51 | 1.22 | 1.68 | 1.30 | 1.38 | 1.34 |
| 48938 | PTPN18 | PTPN18 | protein tyrosine phosphatase, non-receptor | 1.36 | 1.05 | 1.58 | 1.46 | 1.13 | 1.04 |
| | | | type 18 (brain-derived) | | | | | | |
| 50093 | PTPRA | PTP alpha | protein tyrosine phosphatase, receptor type, A | 1.39 | 1.31 | 1.38 | 1.32 | 1.14 | 1.00 |
| 49841 | PTPRCAP | CD45-assoc | protein tyrosine phosphatase, receptor type, C- | 1.33 | 1.31 | 0.77 | 1.25 | 1.04 | 1.00 |
| | | protein | associated protein | | | | | | |
| 47548 | PTPRK | PTPRK | protein tyrosine phosphatase, receptor type, K | 0.65 | 1.37 | 1.21 | 1.11 | 1.11 | 1.02 |
| 48687 | PTPRM | PTPRM | protein tyrosine phosphatase, receptor type, M | 1.34 | 1.31 | 1.05 | 1.01 | 0.98 | 1.00 |
| 47293 | PTPRN | PTPRN | protein tyrosine phosphatase, receptor type, N | 0.96 | 1.00 | 1.66 | 1.33 | 1.08 | 1.00 |
| 48886 | PYGM | | phosphorylase, glycogen; muscle (McArdle | 2.89 | 2.69 | 1.16 | 1.03 | 1.03 | 1.00 |
| | | | syndrome, glycogen storage disease type V) | | | | | | |
| 47653 | RAB10 | RAB10 | RAB10, member RAS oncogene family | 0.72 | 1.32 | 0.77 | 1.09 | 0.96 | 1.00 |
| 47686 | RAB10 | RAB10 | RAB10, member RAS oncogene family | 0.70 | 1.33 | 0.77 | 1.07 | 0.92 | 1.00 |
| 47883 | RAB4A | RAB4 | RAB4, member RAS oncogene family | 1.44 | 1.38 | 0.95 | 1.00 | 0.96 | 1.00 |
| 46924 | RAB9A | RAB9 | RAB9, member RAS oncogene family | 0.47 | 2.04 | 1.00 | 1.00 | 0.98 | 1.00 |
| 47002 | RAC1 | Rac-1 | ras-related C3 botulinum toxin substrate 1 (rho | 0.59 | 1.60 | 0.93 | 1.05 | 0.98 | 1.00 |
| | | | family, small GTP binding protein Rac1) | | | | | | |
| 46969 | RAC1 | Rac-1 | ras-related C3 botulinum toxin substrate 1 (rho | 0.60 | 1.54 | 0.92 | 1.04 | 1.01 | 1.00 |
| | | | family, small GTP binding protein Rac1) | | | | | | |
| 47319 | RAC1 | Rac-1 | ras-related C3 botulinum toxin substrate 1 (rho | 0.68 | 1.38 | 0.93 | 1.03 | 1.05 | 1.00 |
| | | | family, small GTP binding protein Rac1) | | | | | | |
| 48123 | RAD1 | | RAD1 homolog (S. pombe) | 0.80 | 1.08 | 0.72 | 1.33 | 1.04 | 1.00 |
| 49876 | RAD54L | | RAD54-like (S. cerevisiae) | 1.09 | 1.00 | 0.46 | 1.75 | 0.82 | 1.02 |
| 48732 | RALA | RALA | v-ral simian leukemia viral oncogene homolog | 0.57 | 1.48 | 1.03 | 1.00 | 0.90 | 1.00 |
| | | | A (ras related) | | | | | | |
| 48716 | RALA | RALA | v-ral simian leukemia viral oncogene homolog | 0.63 | 1.37 | 1.00 | 1.00 | 0.93 | 1.00 |
| | | | A (ras related) | | | | | | |
| 47446 | RANBP1 | | RAN binding protein 1 | 1.19 | 1.08 | 0.66 | 1.38 | 1.06 | 1.00 |
| 49610 | RANBP2L1 | | hypothetical protein DKFZp434P144 | 0.94 | 1.00 | 1.51 | 1.35 | 1.01 | 1.00 |
| 48542 | RAP1GA1 | | RAP1, GTPase activating protein 1 | 0.62 | 1.31 | 0.63 | 1.24 | 0.83 | 1.00 |
| 47707 | RASA2 | RasGAP | RAS p21 protein activator 2 | 0.84 | 1.00 | 1.41 | 1.30 | 1.05 | 1.00 |
| 47672 | RELA | p65, NFKB3 | v-rel reticuloendotheliosis viral oncogene | 0.51 | 1.76 | 0.99 | 1.00 | 0.78 | 1.19 |
| | | | homolog A, nuclear factor of kappa light | | | | | | |
| | | | polypeptide gene enhancer in B-cells 3, p65 | | | | | | |
| | | | (avian) | | | | | | |
| 47652 | RELA | p65, NFKB3 | v-rel reticuloendotheliosis viral oncogene | 0.53 | 1.60 | 0.97 | 1.00 | 0.80 | 1.01 |
| | | | homolog A, nuclear factor of kappa light | | | | | | |
| | | | polypeptide gene enhancer in B-cells 3, p65 | | | | | | |
| | | | (avian) | | | | | | |
| 49120 | RELB | l-rel | v-rel reticuloendotheliosis viral oncogene | 0.44 | 1.93 | 0.81 | 1.06 | 0.89 | 1.00 |
| | | | homolog B, nuclear factor of kappa light | | | | | | |
| | | | polypeptide gene enhancer in B-cells 3 (avian) | | | | | | |
| 46658 | RELB | l-rel | v-rel reticuloendotheliosis viral oncogene | 0.65 | 1.39 | 0.79 | 1.02 | 0.91 | 1.00 |
| | | | homolog B, nuclear factor of kappa light | | | | | | |
| | | | polypeptide gene enhancer in B-cells 3 (avian) | | | | | | |
| 49087 | RELB | l-rel | v-rel reticuloendotheliosis viral oncogene | 0.43 | 2.02 | 0.85 | 1.00 | 0.88 | 1.00 |
| | | | homolog B, nuclear factor of kappa light | | | | | | |
| | | | polypeptide gene enhancer in B-cells 3 (avian) | | | | | | |
| 49786 | RGS16 | Rgs-16 | regulator of G-protein signalling 16 | 0.47 | 1.65 | 0.89 | 1.01 | 0.96 | 1.00 |
| 48392 | RIPK1 | RIPK, RIP | receptor (TNFRSF)-interacting serine- | 1.52 | 1.00 | 1.85 | 1.77 | 1.31 | 1.16 |
| | | | threonine kinase 1 | | | | | | |
| 46985 | RIPK2 | RIP2, RICK | receptor-interacting serine-threonine kinase 2 | 1.79 | 1.66 | 2.12 | 1.67 | 1.91 | 1.77 |
| 47933 | RIPK2 | RIP2, RICK | receptor-interacting serine-threonine kinase 2 | 0.26 | 3.32 | 1.35 | 1.28 | 0.96 | 1.00 |
| 47774 | RPA2 | | replication protein A2 (32 kD) | 1.06 | 1.00 | 0.64 | 1.40 | 1.11 | 1.00 |
| 49236 | RPA3 | | replication protein A3 (14 kD) | 0.99 | 1.00 | 0.69 | 1.33 | 0.98 | 1.00 |
| 48930 | RPS6KA1 | RSK1 | ribosomal protein S6 kinase, 90 kD, polypeptide 1 | 0.80 | 1.20 | 0.46 | 1.89 | 1.05 | 1.00 |
| 48594 | RPS6KA2 | RPS6KA2 | ribosomal protein S6 kinase, 90 kD, polypeptide 2 | 0.91 | 1.00 | 1.67 | 1.53 | 1.28 | 1.00 |
| 48050 | RPS6KA2 | RPS6KA2 | ribosomal protein S6 kinase, 90 kD, polypeptide 2 | 0.99 | 1.00 | 1.52 | 1.32 | 0.94 | 1.00 |
| 48268 | RPS6KA3 | RPS6KA3 | ribosomal protein S6 kinase, 90 kD, polypeptide 3 | 0.46 | 1.76 | 0.88 | 1.09 | 0.94 | 1.00 |
| 48244 | RPS6KA5 | RPS6KA5 | ribosomal protein S6 kinase, 90 kD, polypeptide 5 | 0.82 | 1.11 | 0.62 | 1.38 | 0.91 | 1.01 |
| 50683 | S100A7 | S100A7 | S100 calcium binding protein A7 (psoriasin 1) | 0.93 | 1.00 | 0.54 | 1.62 | 0.80 | 1.14 |
| 46891 | SAA1 | | serum amyloid A1 | 15.15 | 14.26 | 1.08 | 1.00 | 0.96 | 1.00 |
| 49076 | SARM | KIAA0524 | sterile alpha and HEAT/Armadillo motif protein, | 1.03 | 1.00 | 1.61 | 1.41 | 0.97 | 1.01 |
| | | | ortholog of Drosophila | | | | | | |
| 50441 | SAT | | spermidine/spermine N1-acetyltransferase | 0.64 | 1.52 | 1.01 | 1.00 | 0.93 | 1.00 |
| 49507 | SCNN1A | | sodium channel, nonvoltage-gated 1 alpha | 2.38 | 1.46 | 1.02 | 1.00 | 0.96 | 1.00 |
| 48646 | SDCBP | | syndecan binding protein (syntenin) | 0.57 | 1.73 | 1.06 | 1.00 | 0.91 | 1.00 |
| 48329 | SDHA | | succinate dehydrogenase complex, subunit A, | 1.34 | 1.00 | 1.84 | 1.64 | 1.31 | 1.15 |
| | | | flavoprotein (Fp) | | | | | | |
| 50708 | SELE | E-selectin | selectin E (endothelial adhesion molecule 1) | 0.59 | 1.48 | 1.11 | 1.00 | 0.76 | 1.00 |
| 47522 | SERPINA1 | S, C protease | serine (or cysteine) proteinase inhibitor, clade | 2.10 | 1.92 | 0.89 | 1.06 | 0.90 | 1.06 |
| | | inhibitor | A (alpha-1 antiproteinase, antitrypsin), member 1 | | | | | | |
| 48378 | SERPINA1 | S, C protease | serine (or cysteine) proteinase inhibitor, clade | 3.72 | 2.80 | 0.87 | 1.05 | 0.91 | 1.00 |
| | | inhibitor | A (alpha-1 antiproteinase, antitrypsin), member 1 | | | | | | |
| 46950 | SERPINA3 | | serine (or cysteine) proteinase inhibitor, clade | 8.82 | 8.09 | 1.52 | 1.34 | 1.05 | 1.03 |
| | | | A (alpha-1 antiproteinase, antitrypsin), member 3 | | | | | | |
| 49498 | SERPINA3 | | serine (or cysteine) proteinase inhibitor, clade | 8.93 | 8.67 | 1.43 | 1.34 | 1.07 | 1.00 |
| | | | A (alpha-1 antiproteinase, antitrypsin), member 3 | | | | | | |
| 47166 | SERPINA6 | | serine (or cysteine) proteinase inhibitor, clade | 1.44 | 1.24 | 1.72 | 1.39 | 1.19 | 1.10 |
| | | | A (alpha-1 antiproteinase, antitrypsin), member 6 | | | | | | |
| 47037 | SERPINA6 | | serine (or cysteine) proteinase inhibitor, clade | 1.58 | 1.07 | 1.71 | 1.45 | 1.20 | 1.06 |
| | | | A (alpha-1 antiproteinase, antitrypsin), member 6 | | | | | | |
| 47332 | SERPINB3 | | serine (or cysteine) proteinase inhibitor, clade | 0.40 | 2.32 | 1.38 | 1.24 | 1.04 | 1.00 |
| | | | B (ovalbumin), member 3 | | | | | | |
| 47876 | SERPINB3 | | serine (or cysteine) proteinase inhibitor, clade | 0.34 | 2.48 | 1.35 | 1.21 | 0.99 | 1.00 |
| | | | B (ovalbumin), member 3 | | | | | | |
| 48205 | SERPINB8 | | serine (or cysteine) proteinase inhibitor, clade | 0.36 | 2.62 | 1.27 | 1.18 | 1.11 | 1.03 |
| | | | B (ovalbumin), member 8 | | | | | | |
| 49963 | SERPINB8 | | serine (or cysteine) proteinase inhibitor, clade | 0.38 | 2.40 | 1.24 | 1.17 | 1.00 | 1.00 |
| | | | B (ovalbumin), member 8 | | | | | | |
| 49991 | SERPINE1 | | serine (or cysteine) proteinase inhibitor, clade | 2.87 | 2.69 | 1.95 | 1.83 | 0.90 | 1.01 |
| | | | E (nexin, plasminogen activator inhibitor type | | | | | | |
| | | | 1), member 1 | | | | | | |
| 48269 | SERPINE1 | | serine (or cysteine) proteinase inhibitor, clade | 2.94 | 2.89 | 1.93 | 1.83 | 0.94 | 1.00 |
| | | | E (nexin, plasminogen activator inhibitor type | | | | | | |
| | | | 1), member 1 | | | | | | |
| 48281 | SERPINE2 | | serine (or cysteine) proteinase inhibitor, clade | 0.23 | 3.77 | 1.21 | 1.18 | 0.96 | 1.00 |
| | | | E (nexin, plasminogen activator inhibitor type | | | | | | |
| | | | 1), member 2 | | | | | | |
| 48367 | SERPINE2 | | serine (or cysteine) proteinase inhibitor, clade | 0.27 | 3.45 | 1.29 | 1.14 | 0.93 | 1.00 |
| | | | E (nexin, plasminogen activator inhibitor type | | | | | | |
| | | | 1), member 2 | | | | | | |
| 48400 | SERPINE2 | | serine (or cysteine) proteinase inhibitor, clade | 0.27 | 3.15 | 1.24 | 1.13 | 0.95 | 1.00 |
| | | | E (nexin, plasminogen activator inhibitor type | | | | | | |
| | | | 1), member 2 | | | | | | |
| 46787 | SERPINI1 | | serine (or cysteine) proteinase inhibitor, clade I | 0.57 | 1.50 | 0.64 | 1.21 | 1.10 | 1.00 |
| | | | (neuroserpin), member 1 | | | | | | |
| 47139 | SERPINI1 | | serine (or cysteine) proteinase inhibitor, clade I | 0.59 | 1.57 | 0.83 | 1.00 | 1.20 | 1.00 |
| | | | (neuroserpin), member 1 | | | | | | |
| 48739 | SES2 | | sestrin 2 | 0.63 | 1.44 | 1.70 | 1.48 | 1.10 | 1.00 |
| 49686 | SHC1 | SHC, Shc1 | SHC (Src homology 2 domain containing) | 1.90 | 1.77 | 0.98 | 1.00 | 0.94 | 1.04 |
| | | | transforming protein 1 | | | | | | |
| 47644 | SHC1 | SHC, Shc1 | SHC (Src homology 2 domain containing) | 1.92 | 1.45 | 0.99 | 1.00 | 0.98 | 1.00 |
| | | | transforming protein 1 | | | | | | |
| 48155 | SHMT1 | | serine hydroxymethyltransferase 1 (soluble) | 1.26 | 1.06 | 0.63 | 1.35 | 0.91 | 1.00 |
| 47381 | ShrmL | | Shroom-related protein | 1.87 | 1.69 | 0.80 | 1.06 | 0.98 | 1.00 |
| 47270 | SLC16A4 | | solute carrier family 16 (monocarboxylic acid | 0.68 | 1.35 | 1.20 | 1.05 | 0.88 | 1.06 |
| | | | transporters), member 4 | | | | | | |
| 47211 | SLC26A2 | SLC26A2 | solute carrier family 26 (sulfate transporter), | 5.38 | 5.16 | 1.00 | 1.00 | 1.00 | 1.00 |
| | | | member 2 | | | | | | |
| 50646 | SLC26A4 | | solute carrier family 26, member 4 | 1.00 | 1.00 | 1.98 | 1.70 | 1.00 | 1.00 |
| 48402 | SLC29A1 | ENT1 | solute carrier family 29 (nucleoside | 1.09 | 1.00 | 0.58 | 1.67 | 1.03 | 1.00 |
| | | | transporters), member 1 | | | | | | |
| 48369 | SLC29A1 | ENT1 | solute carrier family 29 (nucleoside | 1.20 | 1.10 | 0.59 | 1.43 | 1.10 | 1.00 |
| | | | transporters), member 1 | | | | | | |
| 47310 | SLC29A1 | ENT1 | solute carrier family 29 (nucleoside | 1.18 | 1.05 | 0.62 | 1.31 | 0.99 | 1.00 |
| | | | transporters), member 1 | | | | | | |
| 47300 | SLPI | SLPI | secretory leukocyte protease inhibitor | 2.05 | 1.91 | 1.03 | 1.00 | 1.03 | 1.00 |
| | | | (antileukoproteinase) | | | | | | |
| 46678 | SLPI | SLPI | secretory leukocyte protease inhibitor | 2.09 | 1.74 | 1.06 | 1.00 | 1.08 | 1.00 |
| | | | (antileukoproteinase) | | | | | | |
| 47838 | SMARCD2 | | SWI/SNF related, matrix associated, actin | 1.48 | 1.32 | 0.87 | 1.01 | 0.94 | 1.00 |
| | | | dependent regulator of chromatin, subfamily d, | | | | | | |
| | | | member 2 | | | | | | |
| 48182 | SNAPC1 | | small nuclear RNA activating complex, | 0.62 | 1.37 | 0.77 | 1.19 | 0.99 | 1.00 |
| | | | polypeptide 1, 43 kD | | | | | | |
| 47418 | SNK | | serum-inducible kinase | 0.36 | 2.54 | 1.03 | 1.00 | 0.97 | 1.00 |
| 48462 | SPRR2C | | small proline-rich protein 2C | 0.75 | 1.04 | 0.37 | 1.94 | 0.96 | 1.00 |
| 49446 | SRC | Src | v-src sarcoma (Schmidt-Ruppin A-2) viral | 0.45 | 1.92 | 0.89 | 1.09 | 1.09 | 1.02 |
| | | | oncogene homolog (avian) | | | | | | |
| 49990 | SRC | Src | v-src sarcoma (Schmidt-Ruppin A-2) viral | 0.45 | 2.02 | 0.93 | 1.00 | 1.05 | 1.02 |
| | | | oncogene homolog (avian) | | | | | | |
| 50450 | STAT1 | STAT1 | signal transducer and activator of transcription | 1.44 | 1.33 | 0.86 | 1.02 | 0.81 | 1.14 |
| | | | 1, 91 kD | | | | | | |
| 49746 | STAT1 | STAT1 | signal transducer and activator of transcription | 1.61 | 1.47 | 0.77 | 1.08 | 0.88 | 1.05 |
| | | | 1, 91 kD | | | | | | |
| 47569 | STAT3 | STAT3 | signal transducer and activator of transcription | 1.88 | 1.68 | 1.71 | 1.45 | 1.42 | 1.34 |
| | | | 3 (acute-phase response factor) | | | | | | |
| 47193 | STAT3 | STAT3 | signal transducer and activator of transcription | 1.83 | 1.67 | 1.73 | 1.55 | 1.47 | 1.18 |
| | | | 3 (acute-phase response factor) | | | | | | |
| 46796 | STC1 | | stanniocalcin 1 | 0.08 | 11.73 | 1.49 | 1.31 | 1.07 | 1.02 |
| 47313 | STK10 | STK10 | serine/threonine kinase 10 | 0.74 | 1.20 | 1.46 | 1.33 | 0.93 | 1.00 |
| 46922 | STK10 | STK10 | serine/threonine kinase 10 | 0.70 | 1.29 | 1.45 | 1.33 | 0.99 | 1.00 |
| 48142 | STOM | | erythrocyte membrane protein band 7.2 | 3.10 | 2.63 | 1.38 | 1.34 | 1.29 | 1.20 |
| | | | (stomatin) | | | | | | |
| 48769 | SUV39H1 | | suppressor of variegation 3-9 homolog 1 | 1.05 | 1.00 | 0.52 | 1.69 | 0.99 | 1.00 |
| | | | (Drosophila) | | | | | | |
| 48505 | T | | T, brachyury homolog (mouse) | 0.52 | 1.82 | 1.03 | 1.00 | 1.18 | 1.00 |
| 47227 | TANK | I-TRAF, TANK | TRAF family member-associated NFKB | 0.67 | 1.41 | 1.04 | 1.00 | 0.92 | 1.00 |
| | | | activator | | | | | | |
| 49372 | TAP1 | | transporter 1, ATP-binding cassette, sub-family | 1.64 | 1.47 | 1.08 | 1.00 | 1.03 | 1.00 |
| | | | B (MDR/TAP) | | | | | | |
| 46613 | TCF12 | TCF12 | transcription factor 12 (HTF4, helix-loop-helix | 0.67 | 1.39 | 1.13 | 1.03 | 0.93 | 1.03 |
| | | | transcription factors 4) | | | | | | |
| 48636 | TCF8 | TCF8 | transcription factor 8 (represses interleukin 2 | 1.39 | 1.36 | 0.85 | 1.07 | 0.99 | 1.00 |
| | | | expression) | | | | | | |
| 48603 | TEAD4 | TEAD4 | TEA domain family member 4 | 1.49 | 1.16 | 1.82 | 1.56 | 1.78 | 1.48 |
| 46991 | TFPI2 | TFPI2 | tissue factor pathway inhibitor 2 | 0.36 | 2.53 | 1.28 | 1.12 | 0.99 | 1.00 |
| 47583 | TGFB2 | TGF, beta2 | transforming growth factor, beta 2 | 0.64 | 1.35 | 0.96 | 1.00 | 0.87 | 1.00 |
| 48907 | TGFB3 | TGF, beta3 | transforming growth factor, beta 3 | 0.68 | 1.35 | 0.80 | 1.17 | 0.98 | 1.00 |
| 47865 | TGFB3 | TGF, beta3 | transforming growth factor, beta 3 | 0.61 | 1.35 | 0.87 | 1.00 | 0.91 | 1.00 |
| 50579 | TGIF | | TGFB-induced factor (TALE family homeobox) | 0.53 | 1.75 | 0.81 | 1.13 | 0.94 | 1.00 |
| 47593 | THBD | | thrombomodulin | 4.04 | 3.85 | 0.97 | 1.00 | 0.94 | 1.00 |
| 47987 | THBS1 | THBS1 | thrombospondin 1 | 4.89 | 3.80 | 0.88 | 1.00 | 1.05 | 1.00 |
| 48358 | THY1 | Thy 1 | Thy-1 cell surface antigen | 0.68 | 1.35 | 0.64 | 1.25 | 0.85 | 1.00 |
| 46859 | TIEG | TGF beta- | TGFB inducible early growth response | 0.66 | 1.34 | 0.98 | 1.00 | 1.11 | 1.05 |
| | | inducible early | | | | | | | |
| | | growth response | | | | | | | |
| 48356 | TIEG | TGF beta- | TGFB inducible early growth response | 0.67 | 1.30 | 1.05 | 1.00 | 1.10 | 1.02 |
| | | inducible early | | | | | | | |
| | | growth response | | | | | | | |
| 49785 | TIMP2 | TIMP2 | tissue inhibitor of metalloproteinase 2 | 0.70 | 1.32 | 1.08 | 1.00 | 0.86 | 1.05 |
| 47778 | TMEPAI | | transmembrane, prostate androgen induced | 0.41 | 2.04 | 1.28 | 1.08 | 0.86 | 1.00 |
| | | | RNA | | | | | | |
| 47918 | TNFAIP1 | TNFAIP1 | tumor necrosis factor, alpha-induced protein 1 | 0.67 | 1.36 | 0.95 | 1.00 | 0.96 | 1.00 |
| | | | (endothelial) | | | | | | |
| 47993 | TNFAIP2 | TNFAIP2 | tumor necrosis factor, alpha-induced protein 2 | 0.53 | 1.73 | 1.14 | 1.05 | 1.10 | 1.05 |
| 47642 | TNFAIP3 | A20, TNFAIP2, Zn | tumor necrosis factor, alpha-induced protein 3 | 0.67 | 1.36 | 1.38 | 1.24 | 0.99 | 1.00 |
| | | finger | | | | | | | |
| 47155 | TNFAIP6 | TNFAIP6 | tumor necrosis factor, alpha-induced protein 6 | 0.42 | 1.86 | 1.44 | 1.35 | 0.95 | 1.00 |
| 46803 | TNFAIP6 | TNFAIP6 | tumor necrosis factor, alpha-induced protein 6 | 0.32 | 2.56 | 1.45 | 1.24 | 0.94 | 1.00 |
| 49272 | TNFRSF10B | DR5 | tumor necrosis factor receptor superfamily, | 0.47 | 1.93 | 1.12 | 1.06 | 1.02 | 1.00 |
| | | | member 10b | | | | | | |
| 49143 | TNFRSF10B | DR5 | tumor necrosis factor receptor superfamily, | 0.46 | 1.97 | 1.12 | 1.04 | 1.05 | 1.00 |
| | | | member 10b | | | | | | |
| 50365 | TNFRSF1A | TNFR1 | tumor necrosis factor receptor superfamily, | 1.09 | 1.01 | 0.65 | 1.41 | 0.91 | 1.04 |
| | | | member 1A | | | | | | |
| 50458 | TNFRSF21 | DR6 | tumor necrosis factor receptor superfamily, | 0.50 | 1.67 | 0.74 | 1.25 | 0.98 | 1.00 |
| | | | member 21 | | | | | | |
| 47684 | TNFRSF21 | DR6 | tumor necrosis factor receptor superfamily, | 0.41 | 2.18 | 0.68 | 1.21 | 0.88 | 1.00 |
| | | | member 21 | | | | | | |
| 47651 | TNFRSF21 | DR6 | tumor necrosis factor receptor superfamily, | 0.36 | 2.37 | 0.72 | 1.15 | 0.93 | 1.00 |
| | | | member 21 | | | | | | |
| 50379 | TNFRSF6 | FAS | tumor necrosis factor receptor superfamily, | 1.65 | 1.20 | 1.69 | 1.66 | 1.27 | 1.10 |
| | | | member 6 | | | | | | |
| 49330 | TNFRSF7 | CD27 | tumor necrosis factor receptor superfamily, | 1.65 | 1.43 | 2.09 | 1.79 | 1.82 | 1.64 |
| | | | member 7 | | | | | | |
| 48395 | TNFRSF7 | CD27 | tumor necrosis factor receptor superfamily, | 1.62 | 1.31 | 1.90 | 1.58 | 1.71 | 1.21 |
| | | | member 7 | | | | | | |
| 48560 | TNFRSF8 | CD30 | tumor necrosis factor receptor superfamily, | 1.36 | 1.13 | 1.52 | 1.32 | 0.97 | 1.00 |
| | | | member 8 | | | | | | |
| 48198 | TNNC1 | | troponin C, slow | 0.73 | 1.31 | 1.11 | 1.03 | 0.96 | 1.00 |
| 49877 | TOB1 | | transducer of ERBB2, 1 | 0.64 | 1.37 | 0.98 | 1.00 | 0.89 | 1.00 |
| 46901 | TP53I3 | | quinone oxidoreductase homolog | 1.68 | 1.59 | 0.99 | 1.00 | 1.01 | 1.00 |
| 48288 | TRAF5 | TRAF5 | TNF receptor-associated factor 5 | 1.61 | 1.21 | 1.83 | 1.71 | 1.60 | 1.33 |
| 48078 | TRIP | TRIP | TRAF interacting protein | 0.90 | 1.00 | 0.56 | 1.58 | 1.01 | 1.00 |
| 49901 | TROAP | | trophinin associated protein (tastin) | 1.01 | 1.00 | 0.50 | 1.79 | 0.94 | 1.01 |
| 49767 | TTRAP | AD022 | TRAF and TNF receptor-associated protein | 0.38 | 2.34 | 1.25 | 1.13 | 0.91 | 1.00 |
| 50738 | TTRAP | AD022 | TRAF and TNF receptor-associated protein | 0.37 | 2.56 | 1.18 | 1.13 | 0.92 | 1.00 |
| 49800 | TTRAP | AD022 | TRAF and TNF receptor-associated protein | 0.38 | 2.45 | 1.20 | 1.06 | 0.89 | 1.00 |
| 47470 | TXNIP | VDUP1 | thioredoxin interacting protein | 2.40 | 1.63 | 1.25 | 1.23 | 1.44 | 1.00 |
| 50575 | UBD | | diubiquitin | 0.56 | 1.42 | 0.87 | 1.00 | 1.00 | 1.00 |
| 48761 | UBE2C | | ubiquitin-conjugating enzyme E2C | 1.84 | 1.70 | 0.70 | 1.27 | 0.93 | 1.00 |
| 49989 | VTN | vitronectin | vitronectin (serum spreading factor, | 0.90 | 1.00 | 0.67 | 1.32 | 0.93 | 1.00 |
| | | | somatomedin B, complement S-protein) | | | | | | |
| 49710 | WSX1 | | class I cytokine receptor | 1.35 | 1.23 | 1.64 | 1.53 | 1.20 | 1.07 |
| 46572 | XRCC2 | | X-ray repair complementing defective repair in | 1.31 | 1.12 | 1.50 | 1.36 | 1.26 | 1.08 |
| | | | Chinese hamster cells 2 | | | | | | |
| 47836 | ZNF140 | | zinc finger protein 140 (clone pHZ-39) | 1.61 | 1.38 | 1.03 | 1.00 | 1.00 | 1.00 |
| 47758 | ZNF184 | | zinc finger protein 184 (Kruppel-like) | 0.50 | 1.75 | 1.07 | 1.00 | 1.04 | 1.00 |
| 48151 | ZWINT | | ZW10 interactor | 0.96 | 1.00 | 0.51 | 1.78 | 1.05 | 1.00 |

IL1 + Macrolide Effects
Genes differentially expressed (≧1.3-fold) in A549 cells in response to at least one macrolide, in presence of IL-1β
Genes in bold have min fold change ≧1.3

| | | | | +IL1b +/− | +IL1b +/− | | +IL1b +/− FMA, | | | | +IL1b +/− Josa, |
|---|---|---|---|---|---|---|---|---|---|---|---|
| | | | | Clari, | Clari, | +IL1b +/− | min | +IL1b +/− Roxi, | +IL1b +/− Roxi, | +IL1b +/− Josa, | min |
| | | | | wtd | min | FMA, | fold | wtd | min | wtd | fold |
| SpotID | GeneSymbol | GeneName | GeneAssignment | means | fold change | wtd means | change | means | fold change | means | change |
| 46987 | | | | 1.97 | 1.76 | 1.66 | 1.58 | 2.37 | 2.10 | 1.95 | 1.72 |
| 48260 | | | | 1.42 | 1.11 | 1.77 | 1.50 | 2.36 | 1.39 | 1.82 | 1.58 |
| 47048 | | | | 1.45 | 1.01 | 1.62 | 1.43 | 1.39 | 1.17 | 1.71 | 1.54 |
| 49343 | | | | 1.78 | 1.53 | 1.72 | 1.42 | 2.00 | 1.64 | 1.79 | 1.53 |
| 48626 | | | | 1.62 | 1.26 | 1.71 | 1.53 | 1.92 | 1.78 | 1.74 | 1.46 |
| 49908 | | | | 1.04 | 1.00 | 2.41 | 1.47 | 0.78 | 1.00 | 2.39 | 1.43 |
| 47003 | | | | 1.70 | 1.32 | 1.84 | 1.60 | 2.02 | 1.56 | 1.67 | 1.43 |
| 49817 | | | | 1.61 | 1.44 | 1.76 | 1.36 | 2.10 | 1.72 | 1.55 | 1.40 |
| 47206 | | | | 1.79 | 1.37 | 1.61 | 1.48 | 1.94 | 1.63 | 1.63 | 1.33 |
| 50375 | | LBC, lymphoid blast | lymphoid blast crisis | 1.41 | 1.25 | 1.35 | 1.21 | 1.38 | 1.15 | 1.50 | 1.33 |
| | | crisis oncogene | oncogene | | | | | | | | |
| 47624 | | | | 1.82 | 1.59 | 1.51 | 1.21 | 1.66 | 1.33 | 1.50 | 1.29 |
| 46609 | | | | 1.68 | 1.40 | 1.64 | 1.38 | 1.87 | 1.56 | 1.47 | 1.27 |
| 50456 | | | | 1.56 | 1.39 | 1.63 | 1.40 | 1.72 | 1.42 | 1.44 | 1.26 |
| 46594 | | | | 1.47 | 1.25 | 1.58 | 1.16 | 1.97 | 1.71 | 1.60 | 1.26 |
| 49612 | | | | 1.40 | 1.00 | 1.51 | 1.31 | 1.81 | 1.43 | 1.42 | 1.16 |
| 50468 | | | | 1.46 | 1.32 | 1.39 | 1.00 | 1.64 | 1.23 | 1.44 | 1.15 |
| 50624 | | | | 1.29 | 1.00 | 2.09 | 1.72 | 0.65 | 1.00 | 1.88 | 1.03 |
| 47315 | | | | 1.43 | 1.07 | 1.52 | 1.17 | 1.69 | 1.45 | 1.25 | 1.00 |
| 46580 | | | | 1.05 | 1.00 | 1.24 | 1.00 | 1.50 | 1.38 | 1.68 | 1.00 |
| 48036 | | | | 1.32 | 1.06 | 1.70 | 1.53 | 1.46 | 1.22 | 1.19 | 1.00 |
| 48580 | | | | 1.41 | 1.05 | 1.68 | 1.41 | 1.42 | 1.14 | 1.22 | 1.00 |
| 49378 | | | | 1.00 | 1.00 | 1.92 | 1.55 | 1.00 | 1.00 | 1.00 | 1.00 |
| 47263 | ACVR2 | | activin A receptor, type II | 1.28 | 1.04 | 1.50 | 1.32 | 1.37 | 1.10 | 1.26 | 1.00 |
| 47535 | ARHGDIA | ARHGDIA | Rho GDP dissociation | 0.91 | 1.00 | 0.81 | 1.00 | 0.82 | 1.15 | 0.75 | 1.30 |
| | | | inhibitor (GDI) alpha | | | | | | | | |
| 48287 | ARHGEF15 | ARHGEF15 | Rho guanine nucleotide | 1.71 | 1.39 | 1.49 | 1.30 | 1.85 | 1.31 | 1.77 | 1.58 |
| | | | exchange factor (GEF) 15 | | | | | | | | |
| 48322 | ARHGEF16 | ARHGEF16 | Rho guanine exchange | 1.73 | 1.28 | 1.25 | 1.10 | 1.82 | 1.51 | 1.49 | 1.34 |
| | | | factor (GEF) 16 | | | | | | | | |
| 46593 | ARHGEF16 | ARHGEF16 | Rho guanine exchange | 1.42 | 1.05 | 1.24 | 1.00 | 1.98 | 1.72 | 1.43 | 1.07 |
| | | | factor (GEF) 16 | | | | | | | | |
| 48516 | ARL6IP | | ADP-ribosylation factor-like | 0.88 | 1.00 | 0.77 | 1.00 | 0.87 | 1.00 | 0.58 | 1.51 |
| | | | 6 interacting protein | | | | | | | | |
| 46817 | ATF3 | ATF3 | activating transcription | 0.94 | 1.00 | 1.39 | 1.17 | 1.31 | 1.15 | 1.67 | 1.31 |
| | | | factor 3 | | | | | | | | |
| 47568 | ATF7 | ATF7 | activating transcription | 1.73 | 1.48 | 1.66 | 1.51 | 2.26 | 1.80 | 1.64 | 1.43 |
| | | | factor 7 | | | | | | | | |
| 47691 | BATF | B-ATF | basic leucine zipper | 1.22 | 1.00 | 1.54 | 1.44 | 1.68 | 1.27 | 0.85 | 1.12 |
| | | | transcription factor, ATF- | | | | | | | | |
| | | | like | | | | | | | | |
| 48679 | BCL3 | Bcl3 | B-cell CLL/lymphoma 3 | 1.29 | 1.20 | 1.41 | 1.32 | 1.61 | 1.36 | 1.49 | 1.20 |
| 50489 | BPAG1 | BPAG1 | bullous pemphigoid | 1.11 | 1.00 | 1.18 | 1.00 | 1.19 | 1.13 | 1.75 | 1.41 |
| | | | antigen 1 (230/240 kD) | | | | | | | | |
| 46854 | BTN3A2 | BTN3A2 | butyrophilin, subfamily 3, | 1.74 | 1.56 | 1.67 | 1.41 | 2.17 | 1.81 | 1.86 | 1.71 |
| | | | member A2 | | | | | | | | |
| 50721 | CASP4 | CASP4 | caspase 4, apoptosis- | 1.27 | 1.00 | 1.29 | 1.18 | 1.19 | 1.10 | 1.50 | 1.44 |
| | | | related cysteine protease | | | | | | | | |
| 47007 | CASP4 | CASP4 | caspase 4, apoptosis- | 1.34 | 1.14 | 1.25 | 1.15 | 1.15 | 1.00 | 1.52 | 1.37 |
| | | | related cysteine protease | | | | | | | | |
| 49807 | CASP4 | CASP4 | caspase 4, apoptosis- | 1.34 | 1.22 | 1.28 | 1.07 | 1.24 | 1.15 | 1.40 | 1.31 |
| | | | related cysteine protease | | | | | | | | |
| 50372 | CASP6 | caspase-6 | caspase 6, apoptosis- | 1.73 | 1.58 | 1.64 | 1.42 | 1.71 | 1.23 | 1.52 | 1.42 |
| | | | related cysteine protease | | | | | | | | |
| 49806 | CCNA2 | cyclin A2 | cyclin A2 | 0.62 | 1.13 | 0.67 | 1.37 | 0.62 | 1.23 | 0.50 | 1.69 |
| 49773 | CCNA2 | cyclin A2 | cyclin A2 | 0.65 | 1.00 | 0.74 | 1.13 | 0.65 | 1.29 | 0.50 | 1.65 |
| 49118 | CCNA2 | cyclin A2 | cyclin A2 | 0.65 | 1.14 | 0.65 | 1.35 | 0.62 | 1.26 | 0.58 | 1.35 |
| 49085 | CCNA2 | cyclin A2 | cyclin A2 | 0.65 | 1.03 | 0.76 | 1.08 | 0.66 | 1.24 | 0.55 | 1.34 |
| 48015 | CCND3 | Cyclin D3 | cyclin D3 | 0.74 | 1.23 | 0.79 | 1.07 | 0.74 | 1.04 | 0.65 | 1.44 |
| 47025 | CCR7 | CCR7 | chemokine (C—C motif) | 0.90 | 1.00 | 1.84 | 1.11 | 1.72 | 1.33 | 1.41 | 1.00 |
| | | | receptor 7 | | | | | | | | |
| 48042 | CD4 | CD4 | CD4 antigen (p55) | 1.49 | 1.31 | 1.30 | 1.00 | 1.49 | 1.00 | 1.23 | 1.00 |
| 50229 | CDC2 | | cell division cycle 2, G1 to | 0.70 | 1.05 | 0.79 | 1.03 | 0.74 | 1.24 | 0.56 | 1.50 |
| | | | S and G2 to M | | | | | | | | |
| 46603 | CDC42BPB | CDC42BPB | CDC42 binding protein | 1.20 | 1.00 | 1.37 | 1.00 | 1.49 | 1.35 | 1.41 | 1.19 |
| | | | kinase beta (DMPK-like) | | | | | | | | |
| 48631 | CDKN1A | Waf1, Cip1 | cyclin-dependent kinase | 1.55 | 1.44 | 1.15 | 1.08 | 1.32 | 1.19 | 1.22 | 1.09 |
| | | | inhibitor 1A (p21, Cip1) | | | | | | | | |
| 49105 | CDKN3 | cyclin dependent | cyclin-dependent kinase | 0.76 | 1.14 | 0.86 | 1.00 | 0.82 | 1.09 | 0.66 | 1.35 |
| | | kinase inhibitor 3 | inhibitor 3 (CDK2- | | | | | | | | |
| | | | associated dual specificity | | | | | | | | |
| | | | phosphatase) | | | | | | | | |
| 46849 | CEBPD | CEBP delta | CCAAT/enhancer binding | 0.76 | 1.30 | 0.78 | 1.10 | 0.68 | 1.32 | 0.72 | 1.32 |
| | | | protein (C/EBP), delta | | | | | | | | |
| 47891 | CEBPD | CEBP delta | CCAAT/enhancer binding | 0.77 | 1.23 | 0.69 | 1.37 | 0.68 | 1.36 | 0.74 | 1.13 |
| | | | protein (C/EBP), delta | | | | | | | | |
| 48313 | CENPA | | centromere protein A | 0.78 | 1.02 | 0.74 | 1.14 | 0.79 | 1.23 | 0.47 | 1.63 |
| | | | (17 kD) | | | | | | | | |
| 50644 | CEP2 | | centrosomal protein 2 | 1.58 | 1.26 | 1.56 | 1.41 | 1.49 | 1.19 | 1.35 | 1.12 |
| 49645 | CX3CL1 | fractalkine | small inducible cytokine | 1.13 | 1.00 | 1.35 | 1.31 | 1.07 | 1.00 | 1.50 | 1.35 |
| | | | subfamily D (Cys-X3-Cys), | | | | | | | | |
| | | | member 1 (fractalkine, | | | | | | | | |
| | | | neurotactin) | | | | | | | | |
| 49492 | CX3CL1 | fractalkine | small inducible cytokine | 1.09 | 1.00 | 1.45 | 1.36 | 0.97 | 1.00 | 1.15 | 1.00 |
| | | | subfamily D (Cys-X3-Cys), | | | | | | | | |
| | | | member 1 (fractalkine, | | | | | | | | |
| | | | neurotactin) | | | | | | | | |
| 49433 | DDIT3 | CHOP | DNA-damage-inducible | 1.82 | 1.43 | 1.31 | 1.16 | 1.56 | 1.33 | 1.52 | 1.40 |
| | | | transcript 3 | | | | | | | | |
| 50132 | DDIT3 | CHOP | DNA-damage-inducible | 1.70 | 1.43 | 1.24 | 1.10 | 1.53 | 1.35 | 1.53 | 1.35 |
| | | | transcript 3 | | | | | | | | |
| 48736 | DUSP5 | DUSP5 | dual specificity | 1.66 | 1.33 | 1.57 | 1.41 | 1.62 | 1.28 | 1.87 | 1.46 |
| | | | phosphatase 5 | | | | | | | | |
| 49777 | DUSP6 | DUSP6 | dual specificity | 1.46 | 1.39 | 1.11 | 1.04 | 1.29 | 1.00 | 1.42 | 1.20 |
| | | | phosphatase 6 | | | | | | | | |
| 49810 | DUSP6 | DUSP6 | dual specificity | 1.46 | 1.39 | 1.12 | 1.04 | 1.32 | 1.20 | 1.34 | 1.17 |
| | | | phosphatase 6 | | | | | | | | |
| 48032 | ECT2 | ECT2 | epithelial cell transforming | 0.62 | 1.33 | 0.64 | 1.17 | 0.71 | 1.04 | 0.64 | 1.37 |
| | | | sequence 2 oncogene | | | | | | | | |
| 50140 | ECT2 | ECT2 | epithelial cell transforming | 0.90 | 1.00 | 0.72 | 1.04 | 0.70 | 1.33 | 0.63 | 1.36 |
| | | | sequence 2 oncogene | | | | | | | | |
| 46821 | ELA2 | elastase-2 | elastase 2, neutrophil | 1.25 | 1.20 | 1.29 | 1.22 | 1.44 | 1.13 | 1.69 | 1.51 |
| 46599 | ERBB3 | ErbB3 | v-erb-b2 erythroblastic | 1.77 | 1.27 | 1.79 | 1.66 | 1.37 | 1.17 | 1.85 | 1.54 |
| | | | leukemia viral oncogene | | | | | | | | |
| | | | homolog 3 (avian) | | | | | | | | |
| 47730 | ERBB3 | ErbB3 | v-erb-b2 erythroblastic | 1.55 | 1.13 | 1.42 | 1.04 | 1.63 | 1.15 | 1.45 | 1.32 |
| | | | leukemia viral oncogene | | | | | | | | |
| | | | homolog 3 (avian) | | | | | | | | |
| 49230 | ESDN | | endothelial and smooth | 0.67 | 1.34 | 0.69 | 1.18 | 0.74 | 1.14 | 0.67 | 1.30 |
| | | | muscle cell-derived | | | | | | | | |
| | | | neuropilin-like protein | | | | | | | | |
| 48998 | FADS1 | | fatty acid desaturase 1 | 1.80 | 1.66 | 0.97 | 1.01 | 1.59 | 1.45 | 0.96 | 1.01 |
| 50717 | FCER1G | FCER1G | Fc fragment of IgE, high | 1.00 | 1.00 | 1.36 | 1.30 | 0.90 | 1.00 | 0.96 | 1.00 |
| | | | affinity I, receptor for; | | | | | | | | |
| | | | gamma polypeptide | | | | | | | | |
| 49529 | FGA | | fibrinogen, A alpha | 0.74 | 1.00 | 0.84 | 1.04 | 0.75 | 1.16 | 0.66 | 1.37 |
| | | | polypeptide | | | | | | | | |
| 48520 | FISH | | KIAA0418 gene product | 1.27 | 1.11 | 1.30 | 1.00 | 1.35 | 1.23 | 1.43 | 1.31 |
| 49892 | FLJ21918 | | hypothetical protein | 1.00 | 1.00 | 2.04 | 1.52 | 1.00 | 1.00 | 1.00 | 1.00 |
| | | | FLJ21918 | | | | | | | | |
| 48623 | FOP | FOP | FGFR1 oncogene partner | 1.01 | 1.00 | 0.74 | 1.17 | 0.88 | 1.00 | 0.64 | 1.34 |
| 50681 | FOXM1 | FOXM1 | forkhead box M1 | 0.75 | 1.07 | 0.88 | 1.11 | 0.80 | 1.18 | 0.62 | 1.39 |
| 48219 | FOXM1 | FOXM1 | forkhead box M1 | 0.68 | 1.28 | 0.86 | 1.14 | 0.78 | 1.15 | 0.65 | 1.35 |
| 47923 | FRAP1 | FRAP1, MTOR | FK506 binding protein 12- | 1.18 | 1.00 | 1.59 | 1.47 | 1.67 | 1.46 | 1.72 | 1.40 |
| | | | rapamycin associated | | | | | | | | |
| | | | protein 1 | | | | | | | | |
| 46725 | FRZB | | frizzled-related protein | 0.37 | 1.00 | 3.36 | 1.75 | 0.86 | 1.00 | 1.75 | 1.00 |
| 50318 | FTL | | ferritin, light polypeptide | 0.73 | 1.30 | 0.79 | 1.00 | 1.26 | 1.05 | 0.88 | 1.00 |
| 49675 | GAB2 | GAB2 | GRB2-associated binding | 2.02 | 1.72 | 1.91 | 1.73 | 1.70 | 1.18 | 1.75 | 1.45 |
| | | | protein 2 | | | | | | | | |
| 48185 | GAGED2 | | G antigen, family D, 2 | 1.20 | 1.12 | 1.15 | 1.04 | 1.18 | 1.13 | 1.42 | 1.32 |
| 47716 | GATA3 | GATA-3 | GATA binding protein 3 | 0.38 | 1.64 | 1.14 | 1.00 | 1.37 | 1.19 | 1.14 | 1.00 |
| 46660 | GFR | KIAA0277 | guanine nucleotide | 1.00 | 1.00 | 2.37 | 1.39 | 1.00 | 1.00 | 1.01 | 1.00 |
| | | | exchange factor for Rap1; | | | | | | | | |
| | | | M-Ras-regulated GEF | | | | | | | | |
| 50653 | GM2A | | GM2 ganglioside activator | 1.46 | 1.34 | 1.32 | 1.05 | 1.39 | 1.12 | 1.28 | 1.07 |
| | | | protein | | | | | | | | |
| 46936 | GRB10 | GRB10 | growth factor receptor- | 1.38 | 1.31 | 1.11 | 1.00 | 1.40 | 1.19 | 1.28 | 1.21 |
| | | | bound protein 10 | | | | | | | | |
| 50018 | GRB2 | Grb2 | growth factor receptor- | 1.53 | 1.33 | 1.30 | 1.01 | 1.52 | 1.35 | 1.33 | 1.06 |
| | | | bound protein 2 | | | | | | | | |
| 48110 | GREB1 | | KIAA0575 gene product | 1.60 | 1.00 | 1.59 | 1.47 | 1.65 | 1.00 | 1.70 | 1.56 |
| 46778 | HMGB2 | | high-mobility group | 0.89 | 1.05 | 0.93 | 1.01 | 0.86 | 1.11 | 0.68 | 1.34 |
| | | | (nonhistone chromosomal) | | | | | | | | |
| | | | protein 2 | | | | | | | | |
| 49992 | HSPG2 | heparin sulfate | heparan sulfate | 0.64 | 1.34 | 0.65 | 1.41 | 0.69 | 1.32 | 0.71 | 1.15 |
| | | proteoglycan-2 | proteoglycan 2 (perlecan) | | | | | | | | |
| 49804 | ICAM1 | ICAM1 | intercellular adhesion | 1.32 | 1.22 | 1.10 | 1.00 | 1.29 | 1.23 | 1.44 | 1.33 |
| | | | molecule 1 (CD54), human | | | | | | | | |
| | | | rhinovirus receptor | | | | | | | | |
| 50084 | IDI1 | | isopentenyl-diphosphate | 1.85 | 1.77 | 0.95 | 1.00 | 1.39 | 1.35 | 0.92 | 1.07 |
| | | | delta isomerase | | | | | | | | |
| 48961 | IL10RB | IL-10R beta | interleukin 10 receptor, | 1.64 | 1.34 | 1.37 | 1.00 | 1.43 | 1.24 | 1.29 | 1.23 |
| | | | beta | | | | | | | | |
| 47509 | IL13RA2 | IL-13R alpha2 | interleukin 13 receptor, | 0.95 | 1.00 | 1.00 | 1.00 | 1.60 | 1.32 | 1.39 | 1.00 |
| | | | alpha 2 | | | | | | | | |
| 47656 | IL1B | IL-1, beta | interleukin 1, beta | 1.39 | 1.29 | 1.27 | 1.09 | 1.42 | 1.36 | 2.04 | 1.86 |
| 46970 | IL1B | IL-1, beta | interleukin 1, beta | 1.42 | 1.13 | 1.27 | 1.11 | 1.46 | 1.41 | 1.84 | 1.53 |
| 48324 | IL3RA | IL-3R alpha | interleukin 3 receptor, | 1.26 | 1.00 | 1.56 | 1.32 | 1.58 | 1.02 | 2.03 | 1.32 |
| | | | alpha (low affinity) | | | | | | | | |
| 49666 | IL4R | IL-4R alpha | interleukin 4 receptor | 1.37 | 1.07 | 1.67 | 1.49 | 1.91 | 1.54 | 1.86 | 1.66 |
| 47925 | IL4R | IL-4R alpha | interleukin 4 receptor | 1.38 | 1.07 | 1.51 | 1.06 | 1.93 | 1.32 | 1.71 | 1.31 |
| 46865 | IL6R | IL-6R | interleukin 6 receptor | 1.78 | 1.36 | 1.79 | 1.51 | 2.39 | 2.27 | 1.77 | 1.56 |
| 49289 | IL6R | IL-6R | interleukin 6 receptor | 1.75 | 1.41 | 1.89 | 1.61 | 2.10 | 1.78 | 1.71 | 1.52 |
| 49873 | INSIG1 | | insulin induced gene 1 | 2.18 | 1.80 | 0.90 | 1.07 | 1.42 | 1.28 | 1.06 | 1.00 |
| 47186 | IRAK2 | IRAK-2 | interleukin-1 receptor- | 1.11 | 1.00 | 1.11 | 1.00 | 1.50 | 1.30 | 1.26 | 1.06 |
| | | | associated kinase 2 | | | | | | | | |
| 49335 | IRF7 | IRF-7A | interferon regulatory factor 7 | 1.17 | 1.04 | 1.52 | 1.47 | 1.55 | 1.23 | 1.28 | 1.00 |
| 50580 | ISG15 | ISG15, IFN stimulated | interferon-stimulated | 1.72 | 1.47 | 1.73 | 1.40 | 1.75 | 1.24 | 1.59 | 1.46 |
| | | protein | protein, 15 kDa | | | | | | | | |
| 46820 | JAK1 | Jak1 | Janus kinase 1 (a protein | 1.30 | 1.08 | 1.45 | 1.20 | 1.46 | 1.37 | 1.07 | 1.00 |
| | | | tyrosine kinase) | | | | | | | | |
| 49322 | JUNB | JunB | jun B proto-oncogene | 0.78 | 1.15 | 0.68 | 1.15 | 0.71 | 1.03 | 0.61 | 1.37 |
| 49319 | JUNB | JunB | jun B proto-oncogene | 0.86 | 1.06 | 0.67 | 1.42 | 0.76 | 1.06 | 0.70 | 1.26 |
| 50528 | KAI1 | CD82 | kangai 1 (suppression of | 1.46 | 1.27 | 1.44 | 1.00 | 1.58 | 1.33 | 1.32 | 1.00 |
| | | | tumorigenicity 6, prostate; | | | | | | | | |
| | | | CD82 antigen.(R2 | | | | | | | | |
| | | | leukocyte antigen, antigen | | | | | | | | |
| | | | detected by monoclonal | | | | | | | | |
| | | | and antibody IA4)) | | | | | | | | |
| 49805 | KBRAS2 | KBRAS2 | I-kappa-B-interacting Ras- | 1.66 | 1.53 | 1.27 | 1.06 | 1.52 | 1.14 | 1.28 | 1.08 |
| | | | like protein 2 | | | | | | | | |
| 49109 | KBRAS2 | KBRAS2 | I-kappa-B-interacting Ras- | 1.68 | 1.56 | 1.36 | 1.19 | 1.73 | 1.31 | 1.31 | 1.00 |
| | | | like protein 2 | | | | | | | | |
| 48081 | KIAA0220 | | KIAA0220 protein | 1.60 | 1.51 | 1.41 | 1.28 | 1.85 | 1.64 | 1.59 | 1.34 |
| 47562 | KIAA1919 | | KIAA1919 protein | 1.51 | 1.31 | 1.09 | 1.00 | 1.34 | 1.22 | 1.22 | 1.05 |
| 49362 | KRT7 | | keratin 7 | 0.73 | 1.00 | 0.82 | 1.11 | 0.58 | 1.54 | 1.15 | 1.00 |
| 50037 | LAMC2 | LAMC2 | laminin, gamma 2 (nicein | 1.43 | 1.38 | 1.18 | 1.14 | 1.41 | 1.18 | 1.37 | 1.19 |
| | | | (100 kD), kalinin (105 kD), | | | | | | | | |
| | | | BM600 (100 kD), Herlitz | | | | | | | | |
| | | | junctional epidermolysis | | | | | | | | |
| | | | bullosa)) | | | | | | | | |
| 49683 | LEPR | leptin R | leptin receptor | 1.89 | 1.55 | 1.75 | 1.55 | 2.42 | 2.08 | 1.79 | 1.69 |
| 49560 | LST1 | | lymphocyte antigen 117 | 0.76 | 1.00 | 1.56 | 1.32 | 1.18 | 1.00 | 2.28 | 1.43 |
| 47356 | LTB4R2 | | seven transmembrane | 1.13 | 1.00 | 1.46 | 1.31 | 1.52 | 1.00 | 1.25 | 1.00 |
| | | | receptor BLTR2; | | | | | | | | |
| | | | leukotriene B4 receptor | | | | | | | | |
| | | | BLT2 | | | | | | | | |
| 47533 | LTBP4 | LTBP4 | latent transforming growth | 0.70 | 1.13 | 0.90 | 1.00 | 0.89 | 1.00 | 0.65 | 1.38 |
| | | | factor beta binding protein 4 | | | | | | | | |
| 47218 | MAP3K7IP1 | TAB1 | transforming growth factor | 1.72 | 1.46 | 1.63 | 1.45 | 2.13 | 1.77 | 1.76 | 1.41 |
| | | | beta-activated kinase- | | | | | | | | |
| | | | binding protein 1 | | | | | | | | |
| 47001 | MAP3K7IP1 | TAB1 | transforming growth factor | 1.42 | 1.13 | 1.70 | 1.41 | 2.07 | 1.54 | 1.43 | 1.00 |
| | | | beta-activated kinase- | | | | | | | | |
| | | | binding protein 1 | | | | | | | | |
| 47491 | MATN3 | MATN3 | matrilin 3 | 1.29 | 1.05 | 1.20 | 1.11 | 1.46 | 1.40 | 1.28 | 1.12 |
| 50566 | MCC | | mutated in colorectal | 1.31 | 1.16 | 1.51 | 1.00 | 1.78 | 1.42 | 1.34 | 1.00 |
| | | | cancers | | | | | | | | |
| 48966 | MMP10 | MMP10 | matrix metalloproteinase | 1.36 | 1.17 | 0.81 | 1.04 | 1.89 | 1.70 | 1.21 | 1.06 |
| | | | 10 (stromelysin 2) | | | | | | | | |
| 50034 | MMPL1 | MMPL1 | matrix metalloproteinase- | 2.13 | 1.46 | 2.21 | 1.42 | 2.63 | 2.12 | 1.15 | 1.00 |
| | | | like 1 | | | | | | | | |
| 49115 | MMPL1 | MMPL1 | matrix metalloproteinase- | 1.68 | 1.09 | 1.60 | 1.00 | 2.45 | 1.70 | 1.47 | 1.00 |
| | | | like 1 | | | | | | | | |
| 48342 | MMPL1 | MMPL1 | matrix metalloproteinase- | 2.04 | 1.36 | 1.26 | 1.00 | 0.89 | 1.00 | 1.34 | 1.00 |
| | | | like 1 | | | | | | | | |
| 47628 | NF1 | NF1 | neurofibromin 1 | 1.28 | 1.05 | 1.33 | 1.17 | 1.33 | 1.24 | 1.51 | 1.42 |
| | | | (neurofibromatosis, von | | | | | | | | |
| | | | Recklinghausen disease, | | | | | | | | |
| | | | Watson disease) | | | | | | | | |
| 47584 | NFAT5 | NFAT 5 | nuclear factor of activated | 1.28 | 1.19 | 1.28 | 1.19 | 1.39 | 1.33 | 1.34 | 1.17 |
| | | | T-cells 5, tonicity- | | | | | | | | |
| | | | responsive | | | | | | | | |
| 50004 | NGFR | NGFR, TNFRSF16 | nerve growth factor | 0.70 | 1.00 | 1.73 | 1.47 | 0.80 | 1.00 | 1.16 | 1.00 |
| | | | receptor (TNFR | | | | | | | | |
| | | | superfamily, member 16) | | | | | | | | |
| 49460 | NGFR | NGFR, TNFRSF16 | nerve growth factor | 0.81 | 1.00 | 1.55 | 1.35 | 0.73 | 1.00 | 1.15 | 1.00 |
| | | | receptor (TNFR | | | | | | | | |
| | | | superfamily, member 16) | | | | | | | | |
| 49158 | NOS1 | | nitric oxide synthase 1 | 1.72 | 1.42 | 1.42 | 1.08 | 1.80 | 1.43 | 1.25 | 1.00 |
| | | | (neuronal) | | | | | | | | |
| 47902 | P2RY2 | P2RY2 | purinergic receptor P2Y, | 1.66 | 1.45 | 1.00 | 1.00 | 1.36 | 1.24 | 1.22 | 1.11 |
| | | | G-protein coupled, 2 | | | | | | | | |
| 49071 | PARG1 | PARG1 | PTPL1-associated | 0.73 | 1.33 | 0.82 | 1.00 | 0.81 | 1.00 | 0.92 | 1.00 |
| | | | RhoGAP 1 | | | | | | | | |
| 48427 | PDE4B | phosphodiesterase | phosphodiesterase 4B, | 1.44 | 1.23 | 1.28 | 1.19 | 1.44 | 1.39 | 1.48 | 1.22 |
| | | 4B | cAMP-specific | | | | | | | | |
| | | | (phosphodiesterase E4 | | | | | | | | |
| | | | dunce homolog, | | | | | | | | |
| | | | Drosophila) | | | | | | | | |
| 48556 | PDE4B | phosphodiesterase | phosphodiesterase 4B, | 1.46 | 1.27 | 1.18 | 1.09 | 1.44 | 1.41 | 1.50 | 1.21 |
| | | 4B | cAMP-specific | | | | | | | | |
| | | | (phosphodiesterase E4 | | | | | | | | |
| | | | dunce homolog, | | | | | | | | |
| | | | Drosophila) | | | | | | | | |
| 47320 | PI3 | PI3 | protease inhibitor 3, skin- | 1.19 | 1.00 | 1.29 | 1.21 | 1.24 | 1.11 | 1.68 | 1.42 |
| | | | derived (SKALP) | | | | | | | | |
| 49398 | PIGR | | polymeric immunoglobulin | 1.45 | 1.16 | 1.60 | 1.47 | 1.53 | 1.26 | 1.47 | 1.11 |
| | | | receptor | | | | | | | | |
| 48547 | PLA2G1B | phospholipase A2 | phospholipase A2, group | 1.74 | 1.62 | 1.52 | 1.19 | 1.41 | 1.00 | 2.12 | 1.77 |
| | | | IB (pancreas) | | | | | | | | |
| 48195 | PLA2G1B | phospholipase A2 | phospholipase A2, group | 1.48 | 1.13 | 1.79 | 1.55 | 2.06 | 1.82 | 1.74 | 1.50 |
| | | | IB (pancreas) | | | | | | | | |
| 50434 | PLAB | PLAB | prostate differentiation | 0.93 | 1.00 | 0.64 | 1.33 | 0.84 | 1.02 | 0.91 | 1.00 |
| | | | factor | | | | | | | | |
| 47390 | PLCD1 | phopholipase C, delta 1 | phospholipase C, delta 1 | 0.82 | 1.00 | 1.27 | 1.00 | 1.11 | 1.00 | 1.95 | 1.61 |
| 47419 | PLS3 | | plastin 3 (T isoform) | 0.82 | 1.11 | 0.73 | 1.31 | 0.80 | 1.13 | 0.81 | 1.21 |
| 48206 | POU2F1 | | POU domain, class 2, | 1.29 | 1.00 | 1.46 | 1.27 | 1.33 | 1.21 | 1.75 | 1.53 |
| | | | transcription factor 1 | | | | | | | | |
| 49103 | PPID | cyclophilin D | peptidylprolyl isomerase D | 1.72 | 1.52 | 1.47 | 1.32 | 1.72 | 1.37 | 1.60 | 1.39 |
| | | | (cyclophilin D) | | | | | | | | |
| 49413 | PPID | cyclophilin D | peptidylprolyl isomerase D | 1.67 | 1.42 | 1.69 | 1.54 | 1.65 | 1.37 | 1.47 | 1.30 |
| | | | (cyclophilin D) | | | | | | | | |
| 46961 | PPP2R2B | PP2 regulatory | protein phosphatase 2 | 1.25 | 1.04 | 1.33 | 1.16 | 1.77 | 1.64 | 2.09 | 1.62 |
| | | subunit B beta | (formerly 2A), regulatory | | | | | | | | |
| | | | subunit B (PR 52), beta | | | | | | | | |
| | | | isoform | | | | | | | | |
| 48726 | PPP2R2B | PP2 regulatory | protein phosphatase 2 | 1.49 | 1.17 | 1.44 | 1.05 | 1.73 | 1.53 | 1.57 | 1.31 |
| | | subunit B beta | (formerly 2A), regulatory | | | | | | | | |
| | | | subunit B (PR 52), beta | | | | | | | | |
| | | | isoform | | | | | | | | |
| 46994 | PPP2R2B | PP2 regulatory | protein phosphatase 2 | 1.39 | 1.00 | 1.53 | 1.24 | 1.86 | 1.71 | 1.74 | 1.11 |
| | | subunit B beta | (formerly 2A), regulatory | | | | | | | | |
| | | | subunit B (PR 52), beta | | | | | | | | |
| | | | isoform | | | | | | | | |
| 46973 | PRKAR1B | PKA, regulatory | protein kinase, cAMP- | 1.55 | 1.23 | 1.56 | 1.40 | 1.73 | 1.16 | 1.39 | 1.01 |
| | | subunit, type I beta | dependent, regulatory, | | | | | | | | |
| | | | type I, beta | | | | | | | | |
| 47190 | PRKCE | PKC epsilon | protein kinase C, epsilon | 0.91 | 1.00 | 1.30 | 1.11 | 1.15 | 1.00 | 1.86 | 1.44 |
| 46646 | PRKCE | PKC epsilon | protein kinase C, epsilon | 0.73 | 1.00 | 2.01 | 1.40 | 0.98 | 1.00 | 1.60 | 1.03 |
| 49036 | PTK2B | PYK2 | protein tyrosine kinase 2 | 1.34 | 1.24 | 1.39 | 1.04 | 1.55 | 1.31 | 1.25 | 1.16 |
| | | | beta | | | | | | | | |
| 47589 | PTOV1 | | prostate tumor over | 0.84 | 1.14 | 0.73 | 1.32 | 0.83 | 1.00 | 0.75 | 1.25 |
| | | | expressed gene 1 | | | | | | | | |
| 47698 | PTPRJ | PTPRJ | protein tyrosine | 1.38 | 1.12 | 1.17 | 1.11 | 1.38 | 1.31 | 1.22 | 1.00 |
| | | | phosphatase, receptor | | | | | | | | |
| | | | type, J | | | | | | | | |
| 48234 | PTPRZ1 | PTP zeta | protein tyrosine | 1.00 | 1.00 | 1.00 | 1.00 | 1.77 | 1.48 | 1.00 | 1.00 |
| | | | phosphatase, receptor- | | | | | | | | |
| | | | type, Z polypeptide 1 | | | | | | | | |
| 48359 | RIPK1 | RIPK, RIP | receptor (TNFRSF)- | 1.61 | 1.50 | 1.27 | 1.00 | 1.34 | 1.00 | 1.11 | 1.00 |
| | | | interacting serine-threonine | | | | | | | | |
| | | | kinase 1 | | | | | | | | |
| 48392 | RIPK1 | RIPK, RIP | receptor (TNFRSF)- | 1.62 | 1.38 | 1.08 | 1.00 | 1.35 | 1.00 | 1.17 | 1.00 |
| | | | interacting serine-threonine | | | | | | | | |
| | | | kinase 1 | | | | | | | | |
| 46985 | RIPK2 | RIP2, RICK | receptor-interacting serine- | 1.83 | 1.41 | 1.70 | 1.43 | 2.23 | 1.85 | 1.87 | 1.59 |
| | | | threonine kinase 2 | | | | | | | | |
| 48500 | RPL13A | | ribosomal protein L13a | 1.65 | 1.43 | 1.37 | 1.25 | 1.23 | 1.06 | 1.46 | 1.40 |
| 50428 | RPLP0 | | ribosomal protein, large, | 0.73 | 1.31 | 0.84 | 1.00 | 0.77 | 1.00 | 1.31 | 1.00 |
| | | | P0 | | | | | | | | |
| 48594 | RPS6KA2 | RPS6KA2 | ribosomal protein S6 | 1.29 | 1.23 | 1.08 | 1.00 | 1.26 | 1.03 | 1.70 | 1.53 |
| | | | kinase, 90 kD, polypeptide 2 | | | | | | | | |
| 48050 | RPS6KA2 | RPS6KA2 | ribosomal protein S6 | 1.12 | 1.00 | 1.43 | 1.12 | 1.06 | 1.00 | 1.53 | 1.42 |
| | | | kinase, 90 kD, polypeptide 2 | | | | | | | | |
| 46950 | SERPINA3 | | serine (or cysteine) | 1.39 | 1.34 | 1.06 | 1.00 | 1.41 | 1.28 | 1.09 | 1.00 |
| | | | proteinase inhibitor, clade | | | | | | | | |
| | | | A (alpha-1 antiproteinase, | | | | | | | | |
| | | | antitrypsin), member 3 | | | | | | | | |
| 47166 | SERPINA6 | | serine (or cysteine) | 1.33 | 1.04 | 1.48 | 1.36 | 1.66 | 1.05 | 1.38 | 1.00 |
| | | | proteinase inhibitor, clade | | | | | | | | |
| | | | A (alpha-1 antiproteinase, | | | | | | | | |
| | | | antitrypsin), member 6 | | | | | | | | |
| 47332 | SERPINB3 | | serine (or cysteine) | 1.39 | 1.24 | 1.22 | 1.06 | 1.33 | 1.25 | 1.43 | 1.40 |
| | | | proteinase inhibitor, clade | | | | | | | | |
| | | | B (ovalbumin), member 3 | | | | | | | | |
| 47193 | STAT3 | STAT3 | signal transducer and | 1.62 | 1.47 | 1.47 | 1.32 | 1.64 | 1.25 | 1.47 | 1.08 |
| | | | activator of transcription 3 | | | | | | | | |
| | | | (acute-phase response | | | | | | | | |
| | | | factor) | | | | | | | | |
| 47569 | STAT3 | STAT3 | signal transducer and | 1.42 | 1.05 | 1.34 | 1.19 | 2.00 | 1.69 | 1.34 | 1.00 |
| | | | activator of transcription 3 | | | | | | | | |
| | | | (acute-phase response | | | | | | | | |
| | | | factor) | | | | | | | | |
| 46796 | STC1 | | stanniocalcin 1 | 1.41 | 1.34 | 1.05 | 1.00 | 1.47 | 1.34 | 1.64 | 1.51 |
| 48603 | TEAD4 | TEAD4 | TEA domain family | 1.67 | 1.35 | 1.65 | 1.38 | 2.19 | 1.72 | 1.52 | 1.42 |
| | | | member 4 | | | | | | | | |
| 47987 | THBS1 | THBS1 | thrombospondin 1 | 0.66 | 1.17 | 0.87 | 1.00 | 0.68 | 1.32 | 0.61 | 1.26 |
| 47444 | TKT | | transketolase (Wernicke- | 0.83 | 1.16 | 0.76 | 1.16 | 0.82 | 1.00 | 0.70 | 1.38 |
| | | | Korsakoff syndrome) | | | | | | | | |
| 48239 | TLN1 | TLN1 | talin 1 | 1.53 | 1.20 | 1.41 | 1.34 | 1.57 | 1.51 | 1.25 | 1.00 |
| 47155 | TNFAIP6 | TNFAIP6 | tumor necrosis factor, | 1.27 | 1.18 | 1.08 | 1.02 | 1.23 | 1.17 | 1.39 | 1.36 |
| | | | alpha-induced protein 6 | | | | | | | | |
| 50379 | TNFRSF6 | FAS | tumor necrosis factor | 1.40 | 1.08 | 1.41 | 1.04 | 1.57 | 1.38 | 1.22 | 1.13 |
| | | | receptor superfamily, | | | | | | | | |
| | | | member 6 | | | | | | | | |
| 47697 | TNFRSF6B | DcR3 | tumor necrosis factor | 1.44 | 1.14 | 1.24 | 1.11 | 1.84 | 1.52 | 1.31 | 1.06 |
| | | | receptor superfamily, | | | | | | | | |
| | | | member 6b, decoy | | | | | | | | |
| 50491 | TNFRSF6B | DcR3 | tumor necrosis factor | 1.36 | 1.00 | 1.56 | 1.36 | 1.60 | 1.17 | 1.29 | 1.04 |
| | | | receptor superfamily, | | | | | | | | |
| | | | member 6b, decoy | | | | | | | | |
| 49330 | TNFRSF7 | CD27 | tumor necrosis factor | 1.76 | 1.53 | 1.73 | 1.51 | 1.90 | 1.44 | 1.66 | 1.48 |
| | | | receptor superfamily, | | | | | | | | |
| | | | member 7 | | | | | | | | |
| 48395 | TNFRSF7 | CD27 | tumor necrosis factor | 1.53 | 1.30 | 1.50 | 1.09 | 1.76 | 1.55 | 1.39 | 1.03 |
| | | | receptor superfamily, | | | | | | | | |
| | | | member 7 | | | | | | | | |
| 48288 | TRAF5 | TRAF5 | TNF receptor-associated | 1.42 | 1.16 | 1.60 | 1.34 | 1.96 | 1.40 | 1.67 | 1.46 |
| | | | factor 5 | | | | | | | | |
| 49901 | TROAP | | trophinin associated | 0.84 | 1.03 | 0.90 | 1.01 | 0.93 | 1.01 | 0.65 | 1.39 |
| | | | protein (tastin) | | | | | | | | |
| 48485 | UCP1 | | uncoupling protein 1 | 1.00 | 1.00 | 1.59 | 1.15 | 1.70 | 1.32 | 1.03 | 1.00 |
| | | | (mitochondrial, proton | | | | | | | | |
| | | | carrier) | | | | | | | | |
| 49899 | WWOX | | WW domain containing | 0.76 | 1.00 | 1.62 | 1.51 | 1.07 | 1.00 | 0.95 | 1.00 |
| | | | oxidoreductase | | | | | | | | |

APPENDIX D
Effect of FADS1 inhibitor +/− macrolides
Genes in bold have min fold change ≧1.3

| | | | | | +/−FADS1, | +FADS1 +/− Clari, | +FADS1 +/− Clari, | +FADS1 +/− Roxi, | +FADS1 +/− Roxi, |
|---|---|---|---|---|---|---|---|---|---|
| | | | | +/−FADS1, | min fold | wtd | min | wtd | min |
| SpotID | GeneSymbol | GeneName | GeneAssignment | wtd means | change | means | fold change | means | fold change |
| 47003 | | | | 1.43 | 1.29 | 0.70 | 1.27 | 0.66 | 1.49 |
| 46594 | | | | 1.40 | 1.24 | 0.66 | 1.31 | 0.68 | 1.41 |
| 49817 | | | | 1.44 | 1.37 | 0.67 | 1.24 | 0.68 | 1.41 |
| 49343 | | | | 1.35 | 1.29 | 0.69 | 1.23 | 0.70 | 1.40 |
| 48626 | | | | 1.48 | 1.31 | 0.69 | 1.28 | 0.69 | 1.40 |
| 46609 | | | | 1.39 | 1.27 | 0.65 | 1.38 | 0.68 | 1.38 |
| 47315 | | | | 1.18 | 1.00 | 0.68 | 1.23 | 0.68 | 1.35 |
| 46987 | | | | 1.50 | 1.13 | 0.65 | 1.39 | 0.62 | 1.35 |
| 50468 | | | | 1.33 | 1.22 | 0.68 | 1.30 | 0.71 | 1.34 |
| 47624 | | | | 1.41 | 1.39 | 0.71 | 1.29 | 0.72 | 1.29 |
| 49720 | | | | 1.00 | 1.00 | 1.31 | 1.30 | 1.32 | 1.23 |
| 49612 | | | | 1.42 | 1.32 | 0.75 | 1.12 | 0.75 | 1.20 |
| 47206 | | | | 1.41 | 1.10 | 0.67 | 1.39 | 0.67 | 1.13 |
| 48260 | | | | 1.56 | 1.48 | 0.66 | 1.37 | 0.75 | 1.05 |
| 47794 | | | | 1.83 | 1.36 | 0.80 | 1.07 | 0.87 | 1.00 |
| 47744 | | | | 1.70 | 1.41 | 0.75 | 1.00 | 0.79 | 1.00 |
| 48039 | | | | 1.79 | 1.31 | 0.81 | 1.00 | 0.91 | 1.00 |
| 49952 | AADAC | | arylacetamide deacetylase (esterase) | 1.30 | 1.10 | 0.75 | 1.24 | 0.66 | 1.48 |
| 47192 | ARHGAP1 | ARHGAP1 | Rho GTPase activating protein 1 | 1.19 | 1.15 | 0.76 | 1.16 | 0.75 | 1.30 |
| 48322 | ARHGEF16 | ARHGEF16 | Rho guanine exchange factor (GEF) 16 | 1.50 | 1.20 | 0.56 | 1.40 | 0.70 | 1.12 |
| 47568 | ATF7 | ATF7 | activating transcription factor 7 | 1.38 | 1.11 | 0.70 | 1.32 | 0.67 | 1.33 |
| 46854 | BTN3A2 | BTN3A2 | butyrophilin, subfamily 3, member A2 | 1.46 | 1.08 | 0.65 | 1.39 | 0.67 | 1.22 |
| 47471 | C21orf18 | | chromosome 21 open reading frame 18 | 1.61 | 1.31 | 0.85 | 1.00 | 0.84 | 1.03 |
| 47927 | CD14 | CD14 | CD14 antigen | 1.48 | 1.31 | 0.60 | 1.16 | 0.73 | 1.00 |
| 47301 | COL11A2 | COL11A2 | collagen, type XI, alpha 2 | 1.68 | 1.34 | 0.64 | 1.00 | 0.71 | 1.00 |
| 50168 | COL4A5 | COL4A5 | collagen, type IV, alpha 5 (Alport syndrome) | 0.94 | 1.00 | 0.89 | 1.02 | 0.71 | 1.38 |
| 48721 | DAF | CD55 | decay accelerating factor for complement (CD55, | 0.99 | 1.00 | 1.41 | 1.30 | 1.39 | 1.32 |
| | | | Cromer blood group system) | | | | | | |
| 50745 | DNAJB1 | DNAJB1 | DnaJ (Hsp40) homolog, subfmaily B, member 1 | 0.72 | 1.32 | 1.19 | 1.08 | 1.13 | 1.05 |
| 48998 | FADS1 | | fatty acid desaturase 1 | 1.01 | 1.00 | 1.46 | 1.02 | 1.48 | 1.38 |
| 50580 | G1P2 | ISG15, IFN | interferon-stimulated protein, 15 kDa | 1.43 | 1.34 | 0.69 | 1.25 | 0.72 | 1.35 |
| | | stimulated | | | | | | | |
| | | protein | | | | | | | |
| 49675 | GAB2 | GAB2 | GRB2-associated binding protein 2 | 1.19 | 1.17 | 0.84 | 1.09 | 0.63 | 1.37 |
| 46611 | GRB14 | GRB14 | growth factor receptor-bound protein 14 | 0.97 | 1.00 | 0.80 | 1.15 | 0.69 | 1.35 |
| 48110 | GREB1 | | KIAA0575 gene product | 1.20 | 1.03 | 0.66 | 1.31 | 0.72 | 1.27 |
| 49185 | GUCA2B | | guanylate cyclase activator 2B (uroguanylin) | 2.02 | 1.38 | 1.00 | 1.00 | 1.30 | 1.13 |
| 49652 | HSPA8 | HSPA8 | heat shock 70 kD protein 8 | 0.91 | 1.07 | 1.20 | 1.05 | 1.56 | 1.42 |
| 49448 | HSPG2 | heparin sulfate | heparan sulfate proteoglycan 2 (perlecan) | 0.99 | 1.00 | 0.70 | 1.32 | 0.76 | 1.05 |
| | | proteoglycan-2 | | | | | | | |
| 50084 | IDI1 | | isopentenyl-diphosphate delta isomerase | 0.92 | 1.05 | 1.46 | 1.03 | 1.47 | 1.38 |
| 47656 | IL1B | IL-1, beta | interleukin 1, beta | 0.91 | 1.00 | 1.20 | 1.11 | 1.38 | 1.32 |
| 46865 | IL6R | IL-6R | interleukin 6 receptor | 1.42 | 1.33 | 0.66 | 1.22 | 0.73 | 1.29 |
| 49873 | INSIG1 | | insulin induced gene 1 | 0.97 | 1.00 | 1.37 | 1.00 | 1.93 | 1.50 |
| 47212 | ITGB5 | integrin beta 5 | integrin, beta 5 | 0.90 | 1.00 | 1.09 | 1.00 | 1.59 | 1.41 |
| 49167 | KDELR3 | | KDEL (Lys-Asp-Glu-Leu) endoplasmic reticulum | 1.58 | 1.21 | 1.44 | 1.33 | 1.37 | 1.28 |
| | | | protein retention receptor 3 | | | | | | |
| 49362 | KRT7 | | keratin 7 | 0.52 | 1.67 | 0.94 | 1.00 | 0.91 | 1.00 |
| 49683 | LEPR | leptin R | leptin receptor | 1.46 | 1.15 | 0.69 | 1.06 | 0.70 | 1.34 |
| 47001 | MAP3K7IP1 | TAB1 | transforming growth factor beta-activated kinase- | 148 | 1.00 | 0.69 | 1.29 | 0.70 | 1.37 |
| | | | binding protein 1 | | | | | | |
| 47218 | MAP3K7IP1 | TAB1 | transforming growth factor beta-activated kinase- | 1.45 | 1.34 | 0.68 | 1.20 | 0.67 | 1.34 |
| | | | binding protein 1 | | | | | | |
| 48951 | MAPK7 | ERK5 | mitogen-activated protein kinase 7 | 1.33 | 1.31 | 0.86 | 1.04 | 0.80 | 1.22 |
| 48760 | MAPK8IP3 | MAPK8IP3 | mitogen-activated protein kinase 8 interacting | 1.42 | 1.17 | 0.63 | 1.32 | 0.63 | 1.19 |
| | | | protein 3 | | | | | | |
| 50566 | MCC | | mutated in colorectal cancers | 1.43 | 1.34 | 0.68 | 1.22 | 0.75 | 1.21 |
| 49115 | MMPL1 | MMPL1 | matrix metalloproteinase-like 1 | 1.58 | 1.38 | 0.60 | 1.14 | 0.61 | 1.07 |
| 50004 | NGFR | NGFR, | nerve growth factor receptor (TNFR superfamily, | 1.71 | 1.38 | 0.89 | 1.00 | 0.95 | 1.00 |
| | | TNFRSF16 | member 16) | | | | | | |
| 47416 | NTS | | neurotensin | 0.68 | 1.40 | 1.08 | 1.00 | 0.90 | 1.08 |
| 47902 | P2RY2 | P2RY2 | purinergic receptor P2Y, G-protein coupled, 2 | 1.00 | 1.00 | 1.74 | 1.41 | 1.23 | 1.02 |
| 49854 | PI3 | PI3 | protease inhibitor 3, skin-derived (SKALP) | 1.01 | 1.00 | 1.50 | 1.43 | 1.34 | 1.10 |
| 49398 | PIGR | | polymeric immunoglobulin receptor | 1.29 | 1.18 | 0.71 | 1.20 | 0.71 | 1.32 |
| 47971 | PLA2G4A | phospholipase | phospholipase A2, group IVA (cytosolic, calcium- | 1.88 | 1.42 | 0.60 | 1.00 | 0.76 | 1.00 |
| | | A2 | dependent) | | | | | | |
| 49103 | PPID | cyclophilin D | peptidylprolyl isomerase D (cyclophilin D) | 1.37 | 1.25 | 0.72 | 1.27 | 0.73 | 1.33 |
| 49413 | PPID | cyclophilin D | peptidylprolyl isomerase D (cyclophilin D) | 1.48 | 1.29 | 0.71 | 1.27 | 0.73 | 1.32 |
| 46994 | PPP2R2B | PP2 regulatory | protein phosphatase 2 (formerly 2A), regulatory | 1.20 | 1.00 | 0.56 | 1.47 | 0.61 | 1.37 |
| | | subunit B beta | subunit B (PR 52), beta isoform | | | | | | |
| 46961 | PPP2R2B | PP2 regulatory | protein phosphatase 2 (formerly 2A), regulatory | 1.18 | 1.00 | 0.63 | 1.33 | 0.64 | 1.28 |
| | | subunit B beta | subunit B (PR 52), beta isoform | | | | | | |
| 48726 | PPP2R2B | PP2 regulatory | protein phosphatase 2 (formerly 2A), regulatory | 1.31 | 1.00 | 0.60 | 1.45 | 0.65 | 1.16 |
| | | subunit B beta | subunit B (PR 52), beta isoform | | | | | | |
| 46973 | PRKAR1B | PKA, regulatory | protein kinase, cAMP-dependent, regulatory, type I, | 1.19 | 1.07 | 0.67 | 1.32 | 0.70 | 1.40 |
| | | subunit, type I | beta | | | | | | |
| | | beta | | | | | | | |
| 47006 | PRKAR1B | PKA, regulatory | protein kinase, cAMP-dependent, regulatory, type I, | 1.39 | 1.20 | 0.65 | 1.32 | 0.71 | 1.38 |
| | | subunit, type I | beta | | | | | | |
| | | beta | | | | | | | |
| 47109 | PRKG2 | | protein kinase, cGMP-dependent, type II | 1.78 | 1.36 | 1.07 | 1.00 | 0.88 | 1.00 |
| 47176 | PTGES | PTGES | prostaglandin E synthase | 0.88 | 1.01 | 1.16 | 1.00 | 1.43 | 1.36 |
| 46985 | RIPK2 | RIP2, RICK | receptor-interacting serine-threonine kinase 2 | 1.57 | 1.36 | 0.66 | 1.39 | 0.65 | 1.32 |
| 48500 | RPL13A | | ribosomal protein L13a | 1.13 | 1.00 | 0.80 | 1.11 | 0.74 | 1.31 |
| 47876 | SERPINB3 | | serine (or cysteine) proteinase inhibitor, clade B | 1.05 | 1.00 | 1.56 | 1.44 | 1.53 | 1.31 |
| | | | (ovalbumin), member 3 | | | | | | |
| 47332 | SERPINB3 | | serine (or cysteine) proteinase inhibitor, clade B | 0.93 | 1.00 | 1.43 | 1.28 | 1.50 | 1.31 |
| | | | (ovalbumin), member 3 | | | | | | |
| 48711 | SPP1 | SPP1 | secreted phosphoprotein 1 (osteopontin, bone | 0.99 | 1.00 | 1.34 | 1.27 | 1.42 | 1.35 |
| | | | sialoprotein I, early T-lymphocyte activation 1) | | | | | | |
| 47193 | STAT3 | STAT3 | signal transducer and activator of transcription 3 | 1.21 | 1.06 | 0.68 | 1.33 | 0.70 | 1.32 |
| | | | (acute-phase response factor) | | | | | | |
| 47569 | STAT3 | STAT3 | signal transducer and activator of transcription 3 | 1.40 | 1.15 | 0.71 | 1.31 | 0.70 | 1.26 |
| | | | (acute-phase response factor | | | | | | |
| 46622 | TCF7 | TCF7 | transcription factor 7 (T-cell specific, HMG-box) | 1.16 | 1.02 | 0.72 | 1.14 | 0.70 | 1.30 |
| 48603 | TEAD4 | TEAD4 | TEA domain family member 4 | 1.42 | 1.28 | 0.66 | 1.33 | 0.65 | 1.31 |
| 48577 | THBS3 | THBS3 | thrombospondin 3 | 1.48 | 1.31 | 0.75 | 1.00 | 0.79 | 1.00 |
| 49330 | TNFRSF7 | CD27 | tumor necrosis factor receptor superfamily, member 7 | 1.43 | 1.38 | 0.69 | 1.28 | 0.71 | 1.35 |
| 48395 | TNFRSF7 | CD27 | tumor necrosis factor receptor superfamily, member 7 | 1.30 | 1.19 | 0.71 | 1.27 | 0.71 | 1.31 |
| 49609 | TNFSF5 | CD40L | tumor necrosis factor (ligand) superfamily, member | 1.64 | 1.31 | 1.01 | 1.00 | 0.83 | 1.00 |
| | | | 5 (hyper-IgM syndrome) | | | | | | |
| 48198 | TNNC1 | | troponin C, slow | 1.66 | 1.38 | 1.00 | 1.00 | 1.02 | 1.00 |
| 48275 | TRAF2 | TRAF2 | TNF receptor-associated factor 2 | 0.98 | 1.00 | 0.65 | 1.30 | 0.76 | 1.00 |
| 48288 | TRAF5 | TRAF5 | TNF receptor-associated factor 5 | 1.34 | 1.26 | 0.64 | 1.37 | 0.70 | 1.20 |

APPENDIX E
Genes statistically predictive of Clari response
Genes differentially expressed in response to Clari but not the inactive macrolides MAA or Josa

| SpotID | GeneSymbol | GeneName | GeneAssignment | p value | mean group 1 | mean group 2 | N of expts | comparison |
|---|---|---|---|---|---|---|---|---|
| 47705 | MAD4 | MAD4 | Mad4 homolog | 4.38E−09 | 0.97 | 1.42 | 58 | MAA and Josa vs. Clari |
| 49922 | DHCR24 | | 24-dehydrocholesterol reductase | 1.46E−08 | 1.00 | 1.27 | 58 | MAA and Josa vs. Clari |
| 48470 | MCM5 | | MCM5 minichromosome maintenance deficient 5, cell | 8.57E−08 | 0.97 | 0.68 | 58 | MAA and Josa vs. Clari |
| | | | division cycle 46 (S. cerevisiae) | | | | | |
| 49688 | GSK3B | GSK3B | glycogen synthase kinase 3 beta | 1.11E−07 | 1.05 | 1.26 | 58 | MAA and Josa vs. Clari |
| 50550 | PTPRT | PTPRT | protein tyrosine phosphatase, receptor type, T | 1.24E−07 | 0.92 | 1.08 | 57 | MAA and Josa vs. Clari |
| 48998 | FADS1 | | fatty acid desaturase 1 | 1.32E−07 | 1.02 | 1.44 | 58 | MAA and Josa vs. Clari |
| 50639 | PDIR | | for protein disulfide isomeras-related | 1.36E−07 | 0.91 | 1.10 | 57 | MAA and Josa vs. Clari |
| 49715 | HNRPD | | heterogeneous nuclear ribonucleoprotein D (AU-rich element RNA binding | 1.70E−07 | 0.99 | 0.88 | 58 | MAA and Josa vs. Clari |
| | | | protein 1, 37 kD) | | | | | |
| 48333 | DNMT1 | | DNA (cytosine-5-)-methyltransferase 1 | 1.76E−07 | 0.97 | 0.79 | 57 | MAA and Josa vs. Clari |
| 50654 | COPB2 | | coatomer protein complex, subunit beta 2 (beta prime) | 3.59E−07 | 0.99 | 1.12 | 58 | MAA and Josa vs. Clari |
| 49183 | SRRM1 | | serine/arginine repetitive matrix 1 | 4.23E−07 | 0.96 | 0.81 | 58 | MAA and Josa vs. Clari |
| 49380 | SLC35A2 | | solute carrier family 35 (UDP-galactose transporter), member 2 | 4.59E−07 | 0.99 | 1.11 | 58 | MAA and Josa vs. Clari |
| 47755 | KIAA1922 | | KIAA1922 protein | 4.76E−07 | 1.14 | 0.79 | 46 | MAA and Josa vs. Clari |
| 50412 | AGPAT1 | | 1-acylglycerol-3-phosphate O-acyltransferase 1 | 5.85E−07 | 0.99 | 1.12 | 58 | MAA and Josa vs. Clari |
| | | | (lysophosphatidic acid acyltransferase, alpha) | | | | | |
| 48713 | MKNK2 | MNK2 | G protein-coupled receptor kinase 7 | 1.11E−06 | 0.96 | 1.22 | 58 | MAA and Josa vs. Clari |
| 48451 | M6PR | | mannose-6-phosphate receptor (cation dependent) | 1.40E−06 | 0.97 | 1.12 | 58 | MAA and Josa vs. Clari |
| 48530 | APLP2 | | amyloid beta (A4) precursor-like protein 2 | 1.52E−06 | 0.95 | 1.15 | 58 | MAA and Josa vs. Clari |
| 50302 | CDR2 | | cerebellar degeneration-related protein (62 kD) | 1.55E−06 | 0.89 | 1.08 | 56 | MAA and Josa vs. Clari |
| 49347 | HNRPA3 | | heterogeneous nuclear ribonucleoprotein A3 | 1.58E−06 | 1.01 | 0.71 | 55 | MAA and Josa vs. Clari |
| 49360 | TEB4 | | similar to S. cerevisiae SSM4 | 1.91E−06 | 1.10 | 0.88 | 58 | MAA and Josa vs. Clari |
| 48644 | KIAA0323 | | KIAA0323 protein | 1.91E−06 | 1.01 | 1.14 | 58 | MAA and Josa vs. Clari |
| 50084 | IDI1 | | isopentenyl-diphosphate delta isomerase | 1.91E−06 | 0.90 | 1.32 | 58 | MAA and Josa vs. Clari |
| 50332 | DAD1 | | defender against cell death 1 | 1.91E−06 | 1.02 | 1.20 | 58 | MAA and Josa vs. Clari |
| 50405 | CHERP | | calcium homeostasis endoplasmic reticulum protein | 2.07E−06 | 1.00 | 0.87 | 58 | MAA and Josa vs. Clari |
| 49650 | MAP3K10 | MLK2 | mitogen-activated protein kinase kinase kinase 10 | 2.23E−06 | 0.93 | 1.16 | 58 | MAA and Josa vs. Clari |
| 49735 | GSK3B | GSK3B | glycogen synthase kinase 3 beta | 2.23E−06 | 1.02 | 1.13 | 58 | MAA and Josa vs. Clari |
| 47446 | RANBP1 | | RAN binding protein 1 | 2.60E−06 | 1.04 | 0.83 | 58 | MAA and Josa vs. Clari |
| 49705 | PCYT2 | | phosphate cytidylyltransferase 2, ethanolamine | 2.60E−06 | 0.92 | 1.24 | 58 | MAA and Josa vs. Clari |
| 47251 | HKE4 | | HLA class II region expressed gene KE4 | 2.67E−06 | 0.94 | 1.13 | 57 | MAA and Josa vs. Clari |
| 49737 | CHAF1B | | chromatin assembly factor 1, subunit B (p60) | 3.02E−06 | 0.99 | 0.84 | 55 | MAA and Josa vs. Clari |
| 47661 | HSPA9B | HSPA9B | heat shock 70 kD protein 9B (mortalin-2) | 3.03E−06 | 1.10 | 0.90 | 58 | MAA and Josa vs. Clari |
| 48328 | FN1 | FN1 | fibronectin 1 | 3.53E−06 | 1.00 | 0.53 | 58 | MAA and Josa vs. Clari |
| 47947 | SF3B3 | | splicing factor 3b, subunit 3, 130 kD | 3.53E−06 | 1.10 | 0.93 | 58 | MAA and Josa vs. Clari |
| 49032 | GSK3B | GSK3B | glycogen synthase kinase 3 beta | 3.80E−06 | 1.06 | 1.23 | 58 | MAA and Josa vs. Clari |
| 48508 | USP24 | | ubiquitin specific protease 24 | 4.10E−06 | 1.03 | 0.87 | 58 | MAA and Josa vs. Clari |
| 47249 | AKR1A1 | | aldo-keto reductase family 1, member A1 (aldehyde | 4.42E−06 | 0.93 | 1.08 | 58 | MAA and Josa vs. Clari |
| | | | reductase) | | | | | |
| 48807 | UNG | | uracil-DNA glycosylase | 4.42E−06 | 1.03 | 0.91 | 58 | MAA and Josa vs. Clari |
| 47123 | MCM2 | | MCM2 minichromosome maintenance deficient 2, mitotin | 4.47E−06 | 0.97 | 0.78 | 54 | MAA and Josa vs. Clari |
| | | | (S. cerevisiae) | | | | | |
| 50337 | FGFR2 | FGF receptor 2 | fibroblast growth factor receptor 2 (bacteria-expressed | 5.39E−06 | 0.93 | 1.08 | 53 | MAA and Josa vs. Clari |
| | | | kinase, keratinocyte growth factor receptor, craniofacial dysostosis 1, | | | | | |
| | | | Crouzon syndrome, Pfeiffer syndrome, Jackson-Weiss syndrome) | | | | | |
| 50307 | MAX | MAX | MAX protein | 5.94E−06 | 0.96 | 1.10 | 58 | MAA and Josa vs. Clari |
| 48971 | P4HA2 | P4HA2 | procollagen-proline, 2-oxoglutarate 4-dioxygenase | 6.24E−06 | 1.07 | 1.48 | 57 | MAA and Josa vs. Clari |
| | | | (proline 4-hydroxylase), alpha polypeptide II | | | | | |
| 50319 | BMP6 | BMP6 | bone morphogenetic protein 6 | 7.08E−06 | 0.98 | 1.15 | 56 | MAA and Josa vs. Clari |
| 47926 | CCNE2 | cyclin E2 | cyclin E2 | 8.27E−06 | 0.98 | 0.73 | 45 | MAA and Josa vs. Clari |
| 47987 | THBS1 | THBS1 | thrombospondin 1 | 8.43E−06 | 1.02 | 0.60 | 48 | MAA and Josa vs. Clari |
| 50659 | MAX | MAX | MAX protein | 8.57E−06 | 0.95 | 1.10 | 58 | MAA and Josa vs. Clari |
| 49169 | RRS1 | | homolog of yeast ribosome biogenesis regulatory protein | 9.04E−06 | 1.09 | 0.88 | 54 | MAA and Josa vs. Clari |
| | | | RRS1 | | | | | |
| 47212 | ITGB5 | integrin beta 5 | integrin, beta 5 | 9.08E−06 | 0.94 | 1.12 | 57 | MAA and Josa vs. Clari |
| 46991 | TFPI2 | TFPI2 | tissue factor pathway inhibitor 2 | 9.19E−06 | 1.00 | 0.57 | 56 | MAA and Josa vs. Clari |
| 47949 | BCKDK | | branched chain alpha-ketoacid dehydrogenase kinase | 9.21E−06 | 1.07 | 0.94 | 58 | MAA and Josa vs. Clari |
| 47131 | KIAA0251 | | KIAA0251 protein | 9.78E−06 | 0.92 | 1.07 | 57 | MAA and Josa vs. Clari |
| 47950 | DNASE1L1 | | deoxyribonuclease I-like 1 | 9.90E−06 | 0.96 | 1.06 | 58 | MAA and Josa vs. Clari |
| 47939 | INPP5E | | KIAA0123 protein | 9.90E−06 | 0.99 | 0.89 | 58 | MAA and Josa vs. Clari |
| 50294 | TM7SF2 | | transmembrane 7 superfamily member 2 | 1.09E−05 | 1.01 | 1.29 | 54 | MAA and Josa vs. Clari |
| 49144 | PIK3R3 | PI3K regulatory | phosphoinositide-3-kinase, regulatory subunit, | 1.14E−05 | 1.01 | 1.15 | 58 | MAA and Josa vs. Clari |
| | | subunit, p55 | polypeptide 3 (p55, gamma) | | | | | |
| 49989 | VTN | vitronectin | vitronectin (serum spreading factor, somatomedin B, | 1.17E−05 | 1.19 | 0.90 | 45 | MAA and Josa vs. Clari |
| | | | complement S-protein) | | | | | |
| 49511 | MATN2 | | matrilin 2 | 1.19E−05 | 0.93 | 1.17 | 55 | MAA and Josa vs. Clari |
| 47935 | P4HA1 | P4HA1, prolyl-4- | procollagen-proline, 2-oxoglutarate 4-dioxygenase | 1.21E−05 | 0.95 | 1.27 | 56 | MAA and Josa vs. Clari |
| | | hydroxylase alpha1 | (proline 4-hydroxylase), alpha polypeptide I | | | | | |
| 47951 | COX15 | | COX15 homolog, cytochrome c oxidase assembly protein | 1.23E−05 | 1.05 | 0.94 | 58 | MAA and Josa vs. Clari |
| | | | (yeast) | | | | | |
| 48149 | SFRS7 | | splicing factor, arginine/serine-rich 7 (35 kD) | 1.30E−05 | 0.98 | 0.85 | 56 | MAA and Josa vs. Clari |
| 49006 | PRSS25 | | protease, serine, 25 | 1.32E−05 | 1.02 | 1.17 | 58 | MAA and Josa vs. Clari |
| 48693 | HSPBP1 | HSPBP1 | hsp70-interacting protein | 1.32E−05 | 1.04 | 0.87 | 58 | MAA and Josa vs. Clari |
| 46579 | SERPING1 | | serine (or cysteine) proteinase inhibitor, clade G (C1 | 1.41E−05 | 1.03 | 0.86 | 56 | MAA and Josa vs. Clari |
| | | | inhibitor), member 1, (angioedema, hereditary) | | | | | |
| 48348 | FN1 | FN1 | fibronectin 1 | 1.42E−05 | 1.00 | 0.53 | 58 | MAA and Josa vs. Clari |
| 49163 | LGALS3 | | lectin, galactoside-binding, soluble, 3 (galectin 3) | 1.52E−05 | 1.06 | 1.23 | 58 | MAA and Josa vs. Clari |
| 47622 | TRAP1 | TRAP1, hsp90 related | heat shock protein 75 | 1.52E−05 | 1.09 | 0.89 | 58 | MAA and Josa vs. Clari |
| 49172 | YAF2 | | YY1 associated factor 2 | 1.60E−05 | 1.01 | 0.90 | 45 | MAA and Josa vs. Clari |
| 47811 | ENTPD6 | | ectonucleoside triphosphate diphosphohydrolase 6 | 1.63E−05 | 0.93 | 1.11 | 58 | MAA and Josa vs. Clari |
| | | | (putative function) | | | | | |
| 48725 | IKKE | IKK-related K epsilon, | IKK-related kinase epsilon; inducible IkappaB kinase | 1.63E−05 | 0.84 | 0.97 | 58 | MAA and Josa vs. Clari |
| | | IKK-I | | | | | | |
| 50738 | AD022 | AD022 | TRAF and TNF receptor-associated protein | 1.63E−05 | 0.94 | 1.14 | 56 | MAA and Josa vs. Clari |
| 47754 | NBS1 | | Nijmegen breakage syndrome 1 (nibrin) | 1.71E−05 | 1.18 | 0.89 | 57 | MAA and Josa vs. Clari |
| 50099 | CNOT3 | CCR4-NOT | CCR4-NOT transcription complex, subunit 3 | 1.75E−05 | 0.95 | 0.84 | 58 | MAA and Josa vs. Clari |
| | | transcription complex, | | | | | | |
| | | subunit 3 | | | | | | |
| 46882 | HEXA | | hexosaminidase A (alpha polypeptide) | 1.88E−05 | 0.97 | 1.14 | 58 | MAA and Josa vs. Clari |
| 48182 | SNAPC1 | | small nuclear RNA activating complex, polypeptide 1, | 1.89E−05 | 1.00 | 0.87 | 54 | MAA and Josa vs. Clari |
| | | | 43 kD | | | | | |
| 48804 | KIAA0217 | | KIAA0217 protein | 1.89E−05 | 1.01 | 0.85 | 56 | MAA and Josa vs. Clari |
| 48471 | RBM3 | | RNA binding motif protein 3 | 2.16E−05 | 1.06 | 0.87 | 58 | MAA and Josa vs. Clari |
| 47192 | ARHGAP1 | ARHGAP1 | Rho GTPase activating protein 1 | 2.16E−05 | 1.10 | 0.94 | 58 | MAA and Josa vs. Clari |
| 49990 | SRC | Src | v-src sarcoma (Schmidt-Ruppin A-2) viral oncogene | 2.28E−05 | 0.99 | 1.17 | 57 | MAA and Josa vs. Clari |
| | | | homolog (avian) | | | | | |
| 48397 | HIF1A | HIF-1 alpha | hypoxia-inducible factor 1, alpha subunit (basic helix-loop- | 2.31E−05 | 1.09 | 0.89 | 58 | MAA and Josa vs. Clari |
| | | | helix transcription factor) | | | | | |
| 47694 | HSPA9B | HSPA9B | heat shock 70 kD protein 9B (mortalin-2) | 2.31E−05 | 1.09 | 0.90 | 58 | MAA and Josa vs. Clari |
| 47406 | MTHFD1 | | methylenetetrahydrofolate dehydrogenase (NADP+ | 2.31E−05 | 1.12 | 0.93 | 58 | MAA and Josa vs. Clari |
| | | | dependent), methenyltetrahydrofolate cyclohydrolase, | | | | | |
| | | | formyltetrahydrofolate synthetase | | | | | |
| 49364 | ABCE1 | | ATP-binding cassette, sub-family E (OABP), member 1 | 2.31E−05 | 1.06 | 0.92 | 58 | MAA and Josa vs. Clari |
| 49608 | POU4F1 | | POU domain, class 4, transcription factor 1 | 2.31E−05 | 0.97 | 1.05 | 58 | MAA and Josa vs. Clari |
| 48707 | P4HA1 | P4HA1, prolyl-4- | procollagen-proline, 2-oxoglutarate 4-dioxygenase | 2.48E−05 | 0.97 | 1.25 | 58 | MAA and Josa vs. Clari |
| | | hydroxylase alpha1 | (proline 4-hydroxylase), alpha polypeptide I | | | | | |
| 46896 | CDIPT | | CDP-diacylglycerol--inositol 3-phosphatidyltransferase | 2.66E−05 | 0.91 | 1.04 | 58 | MAA and Josa vs. Clari |
| | | | (phosphatidylinositol synthase) | | | | | |
| 49787 | CYC1 | cytochrome c-1 | cytochrome c-1 | 2.66E−05 | 1.09 | 0.90 | 58 | MAA and Josa vs. Clari |
| 50224 | HK2 | | hexokinase 2 | 2.85E−05 | 0.95 | 1.14 | 58 | MAA and Josa vs. Clari |
| 49649 | FGF2 | FGF-2 | fibroblast growth factor 2 (basic) | 2.94E−05 | 1.12 | 0.76 | 44 | MAA and Josa vs. Clari |
| 47535 | ARHGDIA | ARHGDIA | Rho GDP dissociation inhibitor (GDI) alpha | 3.05E−05 | 1.08 | 0.91 | 58 | MAA and Josa vs. Clari |
| 46825 | MKNK2 | MNK2 | G protein-coupled receptor kinase 7 | 3.05E−05 | 0.94 | 1.15 | 58 | MAA and Josa vs. Clari |
| 49028 | BNIP3L | BNIP3L | BCL2/adenovirus E1B 19 kD interacting protein 3-like | 3.05E−05 | 1.03 | 1.25 | 58 | MAA and Josa vs. Clari |
| 47352 | ADAM11 | ADAM11 | a disintegrin and metalloproteinase domain 11 | 3.14E−05 | 0.93 | 1.18 | 32 | MAA and Josa vs. Clari |
| 48006 | BCL2L2 | BCL2L2 | BCL2-like 2 | 3.26E−05 | 0.86 | 1.02 | 58 | MAA and Josa vs. Clari |
| 49177 | NEDD8 | | neural precursor cell expressed, developmentally down- | 3.26E−05 | 0.97 | 1.06 | 58 | MAA and Josa vs. Clari |
| | | | regulated 8 | | | | | |
| 48192 | OPA1 | | optic atrophy 1 (autosomal dominant) | 3.26E−05 | 1.02 | 0.90 | 58 | MAA and Josa vs. Clari |
| 48396 | CNOT2 | CCR4-NOT | CCR4-NOT transcription complex, subunit 2 | 3.26E−05 | 1.04 | 0.91 | 58 | MAA and Josa vs. Clari |
| | | transcription complex, | | | | | | |
| | | subunit 2 | | | | | | |
| 50464 | MMP15 | MMP15 | matrix metalloproteinase 15 (membrane-inserted) | 3.46E−05 | 0.87 | 1.10 | 55 | MAA and Josa vs. Clari |
| 48456 | DDC | | dopa decarboxylase (aromatic L-amino acid | 3.46E−05 | 1.12 | 0.94 | 48 | MAA and Josa vs. Clari |
| | | | decarboxylase) | | | | | |
| 49013 | BBC3 | | Bcl-2 binding component 3 | 3.49E−05 | 0.93 | 1.07 | 58 | MAA and Josa vs. Clari |
| 49481 | LOC55871 | | COBW-like protein | 3.49E−05 | 0.90 | 1.05 | 58 | MAA and Josa vs. Clari |
| 49512 | GC20 | | translation factor sui1 homolog | 3.49E−05 | 1.01 | 1.16 | 58 | MAA and Josa vs. Clari |
| 47995 | PTGER1 | PTGER1 | prostaglandin E receptor 1 (subtype EP1), 42 kD | 3.54E−05 | 1.00 | 0.73 | 43 | MAA and Josa vs. Clari |
| 48160 | ZAP3 | | ZAP3 protein | 3.57E−05 | 1.08 | 0.92 | 53 | MAA and Josa vs. Clari |
| 49618 | CLN2 | | ceroid-lipofuscinosis, neuronal 2, late infantile (Jansky- | 3.74E−05 | 1.03 | 1.15 | 58 | MAA and Josa vs. Clari |
| | | | Bielschowsky disease) | | | | | |
| 49698 | CAT | | catalase | 3.74E−05 | 0.92 | 1.08 | 58 | MAA and Josa vs. Clari |
| 50532 | PRKCBP1 | PRKCBP1 | protein kinase C binding protein 1 | 3.74E−05 | 1.07 | 0.94 | 58 | MAA and Josa vs. Clari |
| 47776 | POLA | | polymerase (DNA directed), alpha | 3.79E−05 | 1.02 | 0.86 | 49 | MAA and Josa vs. Clari |
| 50349 | LOC51295 | ECSIT | ECSIT | 3.92E−05 | 1.01 | 0.78 | 56 | MAA and Josa vs. Clari |
| 49155 | ARTS-1 | | type 1 tumor necrosis factor receptor shedding | 3.97E−05 | 1.11 | 0.81 | 49 | MAA and Josa vs. Clari |
| | | | aminopeptidase regulator | | | | | |
| 46979 | HSPBP1 | HSPBP1 | hsp70-interacting protein | 4.00E−05 | 1.02 | 0.87 | 58 | MAA and Josa vs. Clari |
| 47793 | BET3 | | similar to yeast BET3 (S. cerevisiae) | 4.00E−05 | 0.95 | 1.08 | 58 | MAA and Josa vs. Clari |
| 47937 | CTSB | | cathepsin B | 4.57E−05 | 0.98 | 1.15 | 58 | MAA and Josa vs. Clari |
| 49142 | PPIB | cyclophilin B | Peptidylprolyl isomerase B (cyclophilin B) | 4.57E−05 | 1.03 | 1.18 | 58 | MAA and Josa vs. Clari |
| 50284 | UQCR | | ubiquinol-cytochrome c reductase (6.4 kD) subunit | 4.57E−05 | 0.83 | 1.00 | 58 | MAA and Josa vs. Clari |
| 48521 | ARAF1 | | v-raf murine sarcoma 3611 viral oncogene homolog 1 | 4.57E−05 | 1.12 | 0.95 | 58 | MAA and Josa vs. Clari |
| 49391 | PPP3CB | calcineurin A beta | protein phosphatase 3 (formerly 2B), catalytic subunit, | 4.57E−05 | 1.02 | 0.92 | 58 | MAA and Josa vs. Clari |
| | | | beta isoform (calcineurin A beta) | | | | | |
| 50342 | IKKE | IKK-related K epsilon, | IKK-related kinase epsilon; inducible IkappaB kinase | 4.61E−05 | 0.94 | 1.11 | 56 | MAA and Josa vs. Clari |
| | | IKK-I | | | | | | |
| 49514 | UBC | | ubiquitin C | 4.89E−05 | 0.97 | 1.11 | 58 | MAA and Josa vs. Clari |
| 50643 | PROS1 | | protein S (alpha) | 5.20E−05 | 0.93 | 1.11 | 56 | MAA and Josa vs. Clari |
| 50591 | | | | 5.23E−05 | 1.01 | 1.17 | 58 | MAA and Josa vs. Clari |
| 50216 | | | | 5.40E−05 | 0.95 | 1.05 | 57 | MAA and Josa vs. Clari |
| 48411 | MGEA5 | MGEA5 | meningioma expressed antigen 5 (hyaluronidase) | 5.78E−05 | 0.97 | 1.19 | 57 | MAA and Josa vs. Clari |
| 48914 | TUBB | | tubulin, beta polypeptide | 5.97E−05 | 0.96 | 0.83 | 58 | MAA and Josa vs. Clari |
| 48705 | SIGIRR | SIGIRR | single Ig IL-1R-related molecule | 5.99E−05 | 0.97 | 1.12 | 56 | MAA and Josa vs. Clari |
| 48643 | DIP2 | | KIAA0184 protein | 6.38E−05 | 1.00 | 0.87 | 58 | MAA and Josa vs. Clari |
| 50135 | RRBP1 | RRBP1 | ribosome binding protein 1 homolog 180 kD (dog) | 6.38E−05 | 0.95 | 1.11 | 58 | MAA and Josa vs. Clari |
| 47111 | GOLGB1 | | golgi autoantigen, golgin subfamily b, macrogolgin (with | 6.42E−05 | 1.01 | 1.17 | 56 | MAA and Josa vs. Clari |
| | | | transmembrane signal), 1 | | | | | |
| 47034 | C2orf3 | GCF, TCF-9 | chromosome 2 open reading frame 3 | 7.26E−05 | 1.01 | 0.87 | 50 | MAA and Josa vs. Clari |
| 49815 | CDKN2C | p18 | cyclin-dependent kinase inhibitor 2C (p18, inhibits CDK4) | 7.27E−05 | 1.06 | 0.90 | 58 | MAA and Josa vs. Clari |
| 50067 | MBTPS1 | | membrane-bound transcription factor protease, site 1 | 7.27E−05 | 0.95 | 1.07 | 58 | MAA and Josa vs. Clari |
| 49197 | FUCA1 | | fucosidase, alpha-L-1, tissue | 7.27E−05 | 0.98 | 1.13 | 58 | MAA and Josa vs. Clari |
| 49882 | LAMP2 | | lysosomal-associated membrane protein 2 | 7.76E−05 | 0.99 | 1.17 | 58 | MAA and Josa vs. Clari |
| 49314 | PPIB | cyclophilin B | peptidylprolyl isomerase B (cyclophilin B) | 7.76E−05 | 1.04 | 1.18 | 58 | MAA and Josa vs. Clari |
| 49435 | TRRAP | TRRAP | transformation/transcription domain-associated protein | 7.76E−05 | 1.07 | 0.89 | 58 | MAA and Josa vs. Clari |
| 49446 | SRC | Src | v-src sarcoma (Schmidt-Ruppin A-2) viral oncogene | 8.28E−05 | 1.00 | 1.17 | 58 | MAA and Josa vs. Clari |
| | | | homolog (avian) | | | | | |
| 47056 | NFRKB | | nuclear factor related to kappa B binding protein | 8.28E−05 | 0.96 | 0.82 | 58 | MAA and Josa vs. Clari |
| 48646 | SDCBP | | syndecan binding protein (syntenin) | 8.84E−05 | 0.97 | 1.11 | 58 | MAA and Josa vs. Clari |
| 49233 | APOE | | apolipoprotein E | 9.43E−05 | 0.96 | 1.03 | 58 | MAA and Josa vs. Clari |
| 49374 | | | | 9.43E−05 | 0.97 | 1.04 | 58 | MAA and Josa vs. Clari |
| 47524 | PTK2B | PYK2 | protein tyrosine kinase 2 beta | 1.01E−04 | 1.34 | 1.08 | 58 | MAA and Josa vs. Clari |
| 48921 | ITGAE | CD103 | integrin, alpha E (antigen CD103, human mucosal | 1.06E−04 | 0.85 | 0.99 | 57 | MAA and Josa vs. Clari |
| | | | lymphocyte antigen 1; alpha polypeptide) | | | | | |
| 49490 | VTN | vitronectin | vitronectin (serum spreading factor, somatomedin B, | 1.08E−04 | 1.12 | 0.91 | 44 | MAA and Josa vs. Clari |
| | | | complement S-protein) | | | | | |
| 49761 | BCL2L2 | BCL2L2 | BCL2-like 2 | 1.13E−04 | 0.85 | 1.01 | 57 | MAA and Josa vs. Clari |
| 48672 | CHK | | choline kinase | 1.14E−04 | 0.87 | 1.01 | 58 | MAA and Josa vs. Clari |
| 50399 | DGCR6L | | DiGeorge syndrome critical region gene 6 like | 1.15E−04 | 1.02 | 1.17 | 54 | MAA and Josa vs. Clari |
| 48121 | SCN1B | | sodium channel, voltage-gated, type I, beta polypeptide | 1.20E−04 | 0.90 | 1.07 | 54 | MAA and Josa vs. Clari |
| 50513 | ARHC | RhoC | ras homolog gene family, member C | 1.22E−04 | 0.91 | 1.02 | 58 | MAA and Josa vs. Clari |
| 49744 | USF2 | USF2 | upstream transcription factor 2, c-fos interacting | 1.22E−04 | 0.93 | 1.07 | 58 | MAA and Josa vs. Clari |
| 49930 | KIAA0169 | | KIAA0169 protein | 1.22E−04 | 1.03 | 0.95 | 58 | MAA and Josa vs. Clari |
| 48301 | GCN5L1 | | GCN5 general control of amino-acid synthesis 5-like 1 | 1.30E−04 | 0.93 | 1.02 | 58 | MAA and Josa vs. Clari |
| | | | (yeast) | | | | | |
| 49967 | CCNE2 | cyclin E2 | cyclin E2 | 1.31E−04 | 1.02 | 0.82 | 50 | MAA and Josa vs. Clari |
| 49981 | BMP6 | BMP6 | bone morphogenetic protein 6 | 1.32E−04 | 0.94 | 1.10 | 53 | MAA and Josa vs. Clari |
| 48500 | RPL13A | | ribosomal protein L13a | 1.38E−04 | 1.05 | 0.90 | 58 | MAA and Josa vs. Clari |
| 47911 | YWHAE | 14-3-3 epsilon | tyrosine 3-monooxygenase/tryptophan 5-monooxygenase | 1.38E−04 | 1.04 | 0.95 | 58 | MAA and Josa vs. Clari |
| | | | activation protein, epsilon polypeptide | | | | | |
| 50736 | PRKDC | PRKDC | protein kinase, DNA-activated, catalytic polypeptide | 1.38E−04 | 1.07 | 0.90 | 58 | MAA and Josa vs. Clari |
| 50085 | | | | 1.47E−04 | 1.18 | 1.00 | 58 | MAA and Josa vs. Clari |
| 46679 | KIAA1172 | | pre-mRNA splicing SR protein rA4 | 1.48E−04 | 1.04 | 0.91 | 56 | MAA and Josa vs. Clari |
| 46843 | ARHGDIA | ARHGDIA | Rho GDP dissociation inhibitor (GDI) alpha | 1.57E−04 | 1.08 | 0.91 | 58 | MAA and Josa vs. Clari |
| 47898 | ERF | ERF | Ets2 repressor factor | 1.57E−04 | 1.14 | 1.01 | 58 | MAA and Josa vs. Clari |
| 49915 | ATP2B4 | | ATPase, Ca++ transporting, plasma membrane 4 | 1.57E−04 | 1.23 | 1.03 | 58 | MAA and Josa vs. Clari |
| 49352 | NCBP2 | | nuclear cap binding protein subunit 2, 20 kD | 1.66E−04 | 0.94 | 1.03 | 57 | MAA and Josa vs. Clari |
| 48280 | ARHC | RhoC | ras homolog gene family, member C | 1.67E−04 | 0.90 | 1.01 | 58 | MAA and Josa vs. Clari |
| 48235 | DAXX | Daxx | death-associated protein 6 | 1.67E−04 | 1.02 | 0.93 | 58 | MAA and Josa vs. Clari |
| 49373 | EIF3S9 | | eukaryotic translation initiation factor 3, subunit 9 (eta, | 1.67E−04 | 1.06 | 0.94 | 58 | MAA and Josa vs. Clari |
| | | | 116 kD) | | | | | |
| 49318 | YWHAH | 14-3-3 eta | tyrosine 3-monooxygenase/tryptophan 5-monooxygenase | 1.78E−04 | 1.08 | 0.95 | 58 | MAA and Josa vs. Clari |
| | | | activation protein, eta polypeptide | | | | | |
| 50222 | FLJ13868 | | hypothetical protein FLJ13868 | 1.78E−04 | 0.93 | 1.06 | 58 | MAA and Josa vs. Clari |
| 49524 | AP1G2 | | adaptor-related protein complex 1, gamma 2 subunit | 1.78E−04 | 0.74 | 0.97 | 58 | MAA and Josa vs. Clari |
| 47802 | GOT1 | | glutamic-oxaloacetic transaminase 1, soluble (aspartate | 1.89E−04 | 1.02 | 0.93 | 55 | MAA and Josa vs. Clari |
| | | | aminotransferase 1) | | | | | |
| 48000 | CTSH | | cathepsin H | 1.89E−04 | 0.93 | 1.22 | 58 | MAA and Josa vs. Clari |
| 48233 | MAPK12 | ERK6/SAPK3, p38, | mitogen-activated protein kinase 12 | 1.89E−04 | 0.88 | 1.05 | 58 | MAA and Josa vs. Clari |
| | | gamma | | | | | | |
| 50079 | SCOP | | SCN Circadian Oscillatory Protein (SCOP) | 1.89E−04 | 0.97 | 1.05 | 58 | MAA and Josa vs. Clari |
| 49781 | P4HB | P4HB | procollagen-proline, 2-oxoglutarate 4-dioxygenase | 1.89E−04 | 0.98 | 1.10 | 58 | MAA and Josa vs. Clari |
| | | | (proline 4-hydroxylase), beta polypeptide (protein disulfide | | | | | |
| | | | isomerase; thyroid hormone binding protein p55) | | | | | |
| 49757 | DDOST | | dolichyl-diphosphooligosaccharide-protein | 1.89E−04 | 0.96 | 1.08 | 58 | MAA and Josa vs. Clari |
| | | | glycosyltransferase | | | | | |
| 46935 | METAP2 | | methionyl aminopeptidase 2 | 2.01E−04 | 1.05 | 0.88 | 58 | MAA and Josa vs. Clari |
| 47439 | DCTN1 | | dynactin 1 (p150, glued homolog, Drosophila) | 2.01E−04 | 1.04 | 0.93 | 58 | MAA and Josa vs. Clari |
| 47418 | SNK | | serum-inducible kinase | 2.02E−04 | 1.03 | 0.77 | 56 | MAA and Josa vs. Clari |
| 48415 | SERPINB1 | | serine (or cysteine) proteinase inhibitor, clade B | 2.04E−04 | 1.02 | 1.32 | 38 | MAA and Josa vs. Clari |
| | | | (ovalbumin), member 1 | | | | | |
| 48214 | BAT8 | | HLA-B associated transcript 8 | 2.14E−04 | 1.02 | 1.11 | 58 | MAA and Josa vs. Clari |
| 49747 | RPLP0 | | ribosomal protein, large, P0 | 2.14E−04 | 0.91 | 1.09 | 58 | MAA and Josa vs. Clari |
| 49047 | GLUD1 | | glutamate dehydrogenase 1 | 2.14E−04 | 1.05 | 0.95 | 58 | MAA and Josa vs. Clari |
| 49794 | BCL2L2 | BCL2L2 | BCL2-like 2 | 2.15E−04 | 0.86 | 1.01 | 57 | MAA and Josa vs. Clari |
| 50474 | CEBPA | C/EBP alpha | CCAAT/enhancer binding protein (C/EBP), alpha | 2.15E−04 | 0.97 | 1.29 | 57 | MAA and Josa vs. Clari |
| 49767 | AD022 | AD022 | TRAF and TNF receptor-associated protein | 2.15E−04 | 0.96 | 1.12 | 57 | MAA and Josa vs. Clari |
| 50386 | TFAM | TFAM | transcription factor A, mitochondrial | 2.17E−04 | 1.05 | 0.91 | 53 | MAA and Josa vs. Clari |
| 50689 | GNB5 | G protein, beta 5 | guanine nucleotide binding protein (G protein), beta 5 | 2.21E−04 | 1.00 | 1.19 | 46 | MAA and Josa vs. Clari |
| 50103 | IRF5 | IRF5 | interferon regulatory factor 5 | 2.27E−04 | 0.87 | 1.01 | 58 | MAA and Josa vs. Clari |
| 49007 | TM9SF2 | | transmembrane 9 superfamily member 2 | 2.27E−04 | 1.08 | 1.20 | 58 | MAA and Josa vs. Clari |
| 49546 | GNPI | | glucosamine-6-phosphate isomerase | 2.27E−04 | 1.05 | 1.16 | 58 | MAA and Josa vs. Clari |
| 46838 | PRKAR1A | PKA, regulatory | protein kinase, cAMP-dependent, regulatory, type I, alpha | 2.27E−04 | 1.04 | 0.92 | 58 | MAA and Josa vs. Clari |
| | | subunit, type I alpha | (tissue specific extinguisher 1) | | | | | |
| 49820 | CYC1 | cytochrome c-1 | cytochrome c-1 | 2.27E−04 | 1.05 | 0.90 | 58 | MAA and Josa vs. Clari |
| 47850 | PRLR | prolactin R | prolactin receptor | 2.29E−04 | 1.09 | 0.99 | 55 | MAA and Josa vs. Clari |
| 50452 | SIGIRR | SIGIRR | single Ig IL-1R-related molecule | 2.31E−04 | 0.96 | 1.10 | 55 | MAA and Josa vs. Clari |
| 48266 | GRB2 | Grb2 | growth factor receptor-bound protein 2 | 2.42E−04 | 0.93 | 1.06 | 58 | MAA and Josa vs. Clari |
| 46586 | C12orf8 | | chromosome 12 open reading frame 8 | 2.42E−04 | 0.98 | 1.22 | 58 | MAA and Josa vs. Clari |
| 47354 | ERF | ERF | Ets2 repressor factor | 2.42E−04 | 1.15 | 1.00 | 58 | MAA and Josa vs. Clari |
| 50049 | GNB2L1 | | guanine nucleotide binding protein (G protein), beta | 2.42E−04 | 1.32 | 1.10 | 58 | MAA and Josa vs. Clari |
| | | | polypeptide 2-like 1 | | | | | |
| 48363 | CNOT2 | CCR4-NOT | CCR4-NOT transcription complex, subunit 2 | 2.44E−04 | 1.03 | 0.92 | 57 | MAA and Josa vs. Clari |
| | | transcription complex, | | | | | | |
| | | subunit 2 | | | | | | |
| 50011 | NFYC | NFYC | nuclear transcription factor Y, gamma | 2.49E−04 | 1.10 | 0.95 | 56 | MAA and Josa vs. Clari |
| 48365 | MAPK9 | JNK2 | mitogen-activated protein kinase 9 | 2.73E−04 | 1.04 | 0.92 | 58 | MAA and Josa vs. Clari |
| 49117 | SERPINB1 | | serine (or cysteine) proteinase inhibitor, clade B | 2.73E−04 | 1.01 | 0.84 | 58 | MAA and Josa vs. Clari |
| | | | (ovalbumin), member 1 | | | | | |
| 49580 | PSMC4 | | proteasome (prosome, macropain) 26S subunit, ATPase, 4 | 2.73E−04 | 1.12 | 0.99 | 58 | MAA and Josa vs. Clari |
| 46948 | MAP2K1IP1 | MP1 | mitogen-activated protein kinase kinase 1 interacting | 2.79E−04 | 0.92 | 1.05 | 56 | MAA and Josa vs. Clari |
| | | | protein 1 | | | | | |
| 50755 | MMP2 | MMP2, gelatinase A | matrix metalloproteinase 2 (gelatinase A, 72 kD | 2.79E−04 | 1.02 | 1.14 | 56 | MAA and Josa vs. Clari |
| | | | gelatinase, 72 kD type IV collagenase) | | | | | |
| 47241 | NUBP1 | | nucleotide binding protein 1 (MinD homolog, E. coli) | 2.79E−04 | 1.04 | 0.93 | 55 | MAA and Josa vs. Clari |
| 47592 | SART1 | | squamous cell carcinoma antigen recognised by T cells | 2.90E−04 | 1.01 | 0.89 | 58 | MAA and Josa vs. Clari |
| 47522 | SERPINA1 | S, C protease inhibitor | serine (or cysteine) proteinase inhibitor, clade A (alpha-1 | 2.94E−04 | 1.04 | 0.94 | 57 | MAA and Josa vs. Clari |
| | | | antiproteinase, antitrypsin), member 1 | | | | | |
| 50784 | AKR7A2 | | aldo-ket reductase family 7, member A2 (aflatoxin | 3.01E−04 | 0.95 | 1.03 | 55 | MAA and Jose vs. Clari |
| | | | aldehyde reductase) | | | | | |
| 49992 | HSPG2 | heparin sulfate | heparan sulfate proteoglycan 2 (perlecan) | 3.04E−04 | 0.97 | 0.82 | 46 | MAA and Josa vs. Clari |
| | | proteoglycan-2 | | | | | | |
| 47960 | SLC7A5 | | solute carrier family 7 (cationic amino acid transporter, y+ | 3.08E−04 | 1.21 | 1.03 | 58 | MAA and Jose vs. Clari |
| | | | system member 5 | | | | | |
| 50050 | NDUFA2 | | NADH dehydrogenase (ubiquinone) 1 alpha subcomplex, | 3.08E−04 | 0.90 | 1.03 | 58 | MAA and Josa vs. Clari |
| | | | 2 (8 kD, B8) | | | | | |
| 49577 | LOC54543 | | 6.2 kd protein | 3.08E−04 | 0.88 | 1.05 | 58 | MAA and Jose vs. Clari |
| 49510 | | | | 3.08E−04 | 1.00 | 1.28 | 58 | MAA and Josa vs. Clari |
| 48468 | NRG1 | neuregulin1, neu diff. | neuregulin 1 | 3.23E−04 | 1.00 | 0.76 | 57 | MAA and Josa vs. Clari |
| | | Factor, heregulin | | | | | | |
| 48398 | MAPK9 | JNK2 | mitogen-activated protein kinase 9 | 3.27E−04 | 1.02 | 0.92 | 58 | MAA and Josa vs. Clari |
| 49602 | RAB3GAP | | RAB3 GTPase-ACTIVATING PROTEIN | 3.27E−04 | 1.09 | 0.99 | 58 | MAA and Josa vs. Clari |
| 46927 | ACY1 | | aminoacylase 1 | 3.27E−04 | 0.79 | 1.05 | 58 | MAA and Josa vs. Clari |
| 49241 | MCP | | membrane cofactor protein (CD46, trophoblast- | 3.47E−04 | 0.87 | 1.00 | 58 | MAA and Josa vs. Clari |
| | | | lymphocyte cross-reactive antigen) | | | | | |
| 48990 | KRAS2 | K-Ras | v-Ki-ras2 Kirsten rat sarcoma 2 viral oncogene homolog | 3.52E−04 | 1.03 | 0.90 | 53 | MAA and Josa vs. Clari |
| 48464 | FXYD3 | | FXYD domain-containing ion transport regulator 3 | 3.53E−04 | 1.14 | 0.97 | 57 | MAA and Josa vs. Clari |
| 50029 | ROCK2 | ROCK2 | Rho-associated, coiled-coil containing protein kinase 2 | 3.53E−04 | 1.07 | 0.95 | 57 | MAA and Josa vs. Clari |
| 49800 | AD022 | AD022 | TRAF and TNF receptor-associated protein | 3.53E−04 | 0.97 | 1.12 | 57 | MAA and Josa vs. Clari |
| 48918 | KIAA0514 | | KIAA0514 gene product | 3.59E−04 | 0.99 | 1.11 | 56 | MAA and Josa vs. Clari |
| 47973 | USF2 | USF2 | upstream transcription factor 2, c-fos interacting | 3.64E−04 | 0.96 | 1.08 | 56 | MAA and Josa vs. Clari |
| 49657 | CFL1 | CFL1 | cofilin 1 (non-muscle) | 3.68E−04 | 1.16 | 0.96 | 58 | MAA and Josa vs. Clari |
| 50022 | HIF1A | HIF-1 alpha | hypoxia-inducible factor 1, alpha subunit (basic helix-loop- | 3.68E−04 | 1.07 | 0.91 | 58 | MAA and Josa vs. Clari |
| | | | helix transcription factor) | | | | | |
| 48498 | DIRC2 | | hypothetical protein FLJ14784 | 3.84E−04 | 0.99 | 1.13 | 54 | MAA and Josa vs. Clari |
| 48104 | KIAA0546 | | KIAA0546 protein | 3.91E−04 | 0.99 | 1.10 | 58 | MAA and Josa vs. Clari |
| 46851 | RRBP1 | RRBP1 | ribosome binding protein 1 homolog 180 kD (dog) | 3.91E−04 | 0.97 | 1.09 | 58 | MAA and Josa vs. Clari |
| 50347 | TRAP1 | TRAP1, hsp90 related | heat shock protein 75 | 3.91E−04 | 1.06 | 0.87 | 58 | MAA and Josa vs. Clari |
| 47712 | NR4A2 | TINUR | nuclear receptor subfamily 4, group A, member 2 | 4.01E−04 | 0.96 | 1.24 | 49 | MAA and Josa vs. Clari |
| 48486 | | | | 4.06E−04 | 1.21 | 0.94 | 30 | MAA and Josa vs. Clari |
| 48217 | SF3A3 | spliceosomal protein | splicing factor 3a, subunit 3, 60 kD | 4.14E−04 | 1.05 | 0.96 | 58 | MAA and Josa vs. Clari |
| | | SAP 61 | | | | | | |
| 46648 | ARHGAP1 | ARHGAP1 | Rho GTPase activating protein 1 | 4.14E−04 | 1.07 | 0.95 | 58 | MAA and Josa vs. Clari |
| 49397 | FY | duffy antigen | Duffy blood group | 4.14E−04 | 1.14 | 1.03 | 58 | MAA and Josa vs. Clari |
| 48479 | KIF3C | | kinesin family member 3C | 4.18E−04 | 1.06 | 1.23 | 52 | MAA and Josa vs. Clari |
| 47417 | COG2 | | low density lipoprotein receptor defect C complementing | 4.24E−04 | 1.09 | 0.95 | 50 | MAA and Josa vs. Clari |
| 47609 | CYFIP2 | | cytoplasmic FMRP interacting protein 2 | 4.24E−04 | 1.06 | 0.95 | 57 | MAA and Josa vs. Clari |
| 46988 | EREG | epiregulin | epiregulin | 4.29E−04 | 1.11 | 0.80 | 39 | MAA and Josa vs. Clari |
| 48137 | FRDA | | Friedreich ataxia | 4.33E−04 | 1.03 | 0.88 | 56 | MAA and Josa vs. Clari |
| 50601 | SRP19 | | signal recognition particle 19 kD | 4.33E−04 | 0.95 | 1.08 | 56 | MAA and Josa vs. Clari |
| 47504 | MSH3 | | mutS homolog 3 (E. coli) | 4.35E−04 | 1.09 | 0.98 | 43 | MAA and Josa vs. Clari |
| 48406 | CKLF1 | chemokine-like factor 3 | chemokine-like factor 1 | 4.39E−04 | 0.91 | 1.10 | 58 | MAA and Josa vs. Clari |
| 49873 | INSIG1 | | insulin induced gene 1 | 4.39E−04 | 0.93 | 1.44 | 58 | MAA and Josa vs. Clari |
| 49525 | PIGQ | | phosphatidylinositol glycan, class Q | 4.39E−04 | 0.90 | 1.05 | 58 | MAA and Josa vs. Clari |
| 47181 | RAB2 | RAB2 | RAB2, member RAS oncogene family | 4.66E−04 | 1.01 | 1.09 | 58 | MAA and Josa vs. Clari |
| 49785 | TIMP2 | TIMP2 | tissue inhibitor of metalloproteinase 2 | 4.94E−04 | 1.07 | 1.35 | 58 | MAA and Josa vs. Clari |
| 50632 | PTPRJ | PTPRJ | protein tyrosine phosphatase, receptor type, J | 4.94E−04 | 1.12 | 1.30 | 58 | MAA and Josa vs. Clari |
| 49134 | SYK | Syk | spleen tyrosine kinase | 5.00E−04 | 0.98 | 1.31 | 16 | MAA and Josa vs. Clari |
| 50590 | CKS2 | | CDC28 protein kinase 2 | 5.03E−04 | 0.74 | 0.90 | 55 | MAA and Josa vs. Clari |
| 48640 | IL11RA | IL-11R alpha | interleukin 11 receptor, alpha | 5.04E−04 | 0.79 | 0.99 | 49 | MAA and Josa vs. Clari |
| 47324 | TCF7L2 | TCF7L2 | transcription factor 7-like 2 (T-cell specific, HMG-box) | 5.04E−04 | 1.06 | 0.89 | 49 | MAA and Josa vs. Clari |
| 48360 | CASP3 | caspase-3 | caspase 3, apoptosis-related cysteine protease | 5.08E−04 | 0.88 | 1.02 | 57 | MAA and Josa vs. Clari |
| 47679 | NR4A2 | TINUR | nuclear receptor subfamily 4, group A, member 2 | 5.11E−04 | 0.95 | 1.20 | 49 | MAA and Josa vs. Clari |
| 49654 | PRKCD | PKC, delta | protein kinase C, delta | 5.24E−04 | 0.95 | 1.04 | 58 | MAA and Josa vs. Clari |
| 49818 | TIMP2 | TIMP2 | tissue inhibitor of metalloproteinase 2 | 5.24E−04 | 1.07 | 1.35 | 58 | MAA and Josa vs. Clari |
| 48822 | UGDH | | UDP-glucose dehydrogenase | 5.24E−04 | 0.94 | 0.84 | 58 | MAA and Josa vs. Clari |
| 48004 | CUTL1 | CUTL1 | cut-like 1, CCAAT displacement protein (Drosophila) | 5.27E−04 | 1.22 | 1.00 | 53 | MAA and Josa vs. Clari |
| 50119 | TOLLIP | Tollip | Toll-interacting protein | 5.55E−04 | 1.07 | 1.19 | 58 | MAA and Josa vs. Clari |
| 47027 | TNFRSF5 | CD40 | tumor necrosis factor receptor superfamily, member 5 | 5.55E−04 | 0.94 | 1.06 | 58 | MAA and Josa vs. Clari |
| 48329 | SDHA | | succinate dehydrogenase complex, subunit A, | 5.55E−04 | 1.03 | 0.90 | 58 | MAA and Josa vs. Clari |
| | | | flavoprotein (Fp) | | | | | |
| 50351 | CFL1 | CFL1 | cofilin 1 (non-muscle) | 5.55E−04 | 1.16 | 0.97 | 58 | MAA and Josa vs. Clari |
| 48126 | RY1 | | putative nucleic acid binding protein RY-1 | 5.70E−04 | 1.01 | 0.85 | 53 | MAA and Josa vs. Clari |
| 49394 | CDC25A | | cell division cycle 25A | 5.84E−04 | 1.03 | 0.86 | 53 | MAA and Josa vs. Clari |
| 48620 | XBP1 | XBP-1 | X-box binding protein 1 | 5.88E−04 | 0.98 | 1.10 | 58 | MAA and Josa vs. Clari |
| 47436 | LSM1 | | Lsm1 protein | 5.88E−04 | 0.87 | 1.01 | 58 | MAA and Josa vs. Clari |
| 48952 | ELK1 | Elk-1 | ELK1, member of ETS oncogene family | 5.88E−04 | 0.98 | 1.06 | 58 | MAA and Josa vs. Clari |
| 49434 | CKLF1 | chemokine-like factor 3 | chemokine-like factor 1 | 5.88E−04 | 0.90 | 1.10 | 58 | MAA and Josa vs. Clari |
| 49045 | TUFM | | Tu translation elongation factor, mitochondrial | 5.88E−04 | 1.02 | 0.91 | 58 | MAA and Josa vs. Clari |
| 50365 | TNFRSF1A | TNFR1 | tumor necrosis factor receptor superfamily, member 1A | 6.07E−04 | 0.95 | 1.07 | 57 | MAA and Josa vs. Clari |
| 49196 | | | | 6.12E−04 | 0.99 | 1.18 | 54 | MAA and Josa vs. Clari |
| 49933 | PDE3A | | phosphodiesterase 3A, cGMP-inhibited | 6.15E−04 | 1.10 | 0.89 | 42 | MAA and Josa vs. Clari |
| 47988 | RPN2 | | ribophorin II | 6.22E−04 | 0.93 | 1.03 | 58 | MAA and Josa vs. Clari |
| 48303 | RUVBL1 | | RuvB-like 1 (E. coli) | 6.22E−04 | 1.06 | 0.97 | 58 | MAA and Josa vs. Clari |
| 50576 | GGCX | | gamma-glutamyl Carboxylase | 6.43E−04 | 0.85 | 1.00 | 57 | MAA and Josa vs. Clari |
| 47569 | STAT3 | STAT3 | signal transducer and activator of transcription 3 (acute- | 6.59E−04 | 1.04 | 0.91 | 58 | MAA and Josa vs. Clari |
| | | | phase response factor) | | | | | |
| 47156 | TNFRSF5 | CD40 | tumor necrosis factor receptor superfamily, member 5 | 6.64E−04 | 0.93 | 1.06 | 57 | MAA and Josa vs. Clari |
| 47916 | CCL2 | MCP1 | small inducible cytokine A2 (monocyte chemotactic | 6.75E−04 | 1.15 | 0.63 | 31 | MAA and Josa vs. Clari |
| | | | protein 1) | | | | | |
| 47292 | APOC1 | | apolipoprotein C-I | 6.82E−04 | 0.98 | 1.11 | 57 | MAA and Josa vs. Clari |
| 49947 | RFC1 | | replication factor C (activator 1) 1 (145 kD) | 6.95E−04 | 1.05 | 0.91 | 54 | MAA and Josa vs. Clari |
| 47415 | CETN2 | | centrin, EF-hand protein, 2 | 6.98E−04 | 1.06 | 1.19 | 58 | MAA and Josa vs. Clari |
| 48891 | STK39 | | serine threonine kinase 39 (STE20/SPS1 homolog, yeast) | 7.05E−04 | 1.02 | 0.91 | 50 | MAA and Josa vs. Clari |
| 50430 | PPM1A | | protein phosphatase 1A (formerly 2C), magnesium- | 7.23E−04 | 0.98 | 1.06 | 57 | MAA and Josa vs. Clari |
| | | | dependent, alpha isoform | | | | | |
| 48085 | DC2 | | DC2 protein | 7.38E−04 | 0.99 | 1.08 | 58 | MAA and Josa vs. Clari |
| 49638 | AKT3 | AKT3, PKB | v-akt murine thymoma viral oncogene homolog 3 (protein | 7.38E−04 | 0.99 | 1.16 | 58 | MAA and Josa vs. Clari |
| | | | kinase B, gamma) | | | | | |
| 48926 | UBE2B | | ubiguitin-conjugating enzyme E2B (RAD6 homolog) | 7.38E−04 | 0.96 | 1.07 | 58 | MAA and Josa vs. Clari |
| 49551 | MGC19606 | | similar to HYPOTHETICAL 34.0 KDA PROTEIN ZK795.3 | 7.38E−04 | 1.03 | 0.93 | 58 | MAA and Josa vs. Clari |
| | | | IN CHROMOSOME IV | | | | | |
| 47349 | HSGT1 | | suppressor of S. cerevisiae gcr2 | 7.57E−04 | 1.07 | 0.94 | 56 | MAA and Josa vs. Clari |
| 46650 | P2RY6 | P2RY6 | pyrimidinergic receptor P2Y, G-protein coupled, 6 | 7.67E−04 | 0.95 | 1.14 | 57 | MAA and Josa vs. Clari |
| 49229 | ZXDA | | zinc finger, X-linked, duplicated A | 7.81E−04 | 1.01 | 1.15 | 58 | MAA and Josa vs. Clari |
| 48502 | CLIC4 | | chloride intracellular channel 4 | 7.88E−04 | 1.17 | 1.02 | 49 | MAA and Josa vs. Clari |
| 50764 | KIAA0212 | | KIAA0212 gene product | 7.96E−04 | 0.99 | 1.13 | 56 | MAA and Josa vs. Clari |
| 46938 | ROCK2 | ROCK2 | Rho-associated, coiled-coil containing protein kinase 2 | 8.27E−04 | 1.06 | 0.92 | 58 | MAA and Josa vs. Clari |
| 49893 | CHD4 | | chromodomain helicase DNA binding protein 4 | 8.27E−04 | 1.02 | 0.93 | 58 | MAA and Josa vs. Clari |
| 48841 | TAF6 | | TAF6 RNA polymerase II, TATA box binding protein | 8.27E−04 | 1.06 | 0.95 | 58 | MAA and Josa vs. Clari |
| | | | (TBP)-associated factor, 80 kD | | | | | |
| 49550 | SETDB1 | | SET domain, bifurcated 1 | 8.27E−04 | 1.01 | 0.95 | 58 | MAA and Josa vs. Clari |
| 50267 | IL10RB | IL-10R beta | interleukin 10 receptor, beta | 8.27E−04 | 0.98 | 1.09 | 58 | MAA and Josa vs. Clari |
| 50517 | ARHGAP4 | ARHGAP4 | Rho GTPase activating protein 4 | 8.58E−04 | 0.86 | 1.10 | 49 | MAA and Josa vs. Clari |
| 49728 | GALK2 | | galactokinase 2 | 8.74E−04 | 0.95 | 1.05 | 58 | MAA and Josa vs. Clari |
| 46834 | PAK4 | Pak4 | p21(CDKN1A)-activated kinase 4 | 8.97E−04 | 1.04 | 0.89 | 56 | MAA and Josa vs. Clari |
| 48717 | PPP3CB | calcineurin A beta | protein phosphatase 3 (formerly 2B), catalytic subunit, | 9.07E−04 | 1.09 | 0.95 | 44 | MAA and Josa vs. Clari |
| | | | beta isoform calcineurin A beta) | | | | | |
| 49448 | HSPG2 | heparin sulfate | heparan sulfate proteoglycan 2 (perlecan) | 9.21E−04 | 0.97 | 0.83 | 47 | MAA and Josa vs. Clari |
| | | proteoglycan-2 | | | | | | |
| 47658 | SERPINH2 | | serine (or cysteine) proteinase inhibitor, clade H (heat | 9.25E−04 | 1.03 | 0.95 | 58 | MAA and Josa vs. Clari |
| | | | shock protein 47), member 2 | | | | | |
| 47627 | HARS | | histidyl-tRNA synthetase | 9.25E−04 | 1.04 | 0.97 | 58 | MAA and Josa vs. Clari |
| 50205 | KIAA0007 | | KIAA0007 protein | 9.25E−04 | 0.98 | 0.89 | 58 | MAA and Josa vs. Clari |
| 50407 | SORT1 | | sortilin 1 | 9.25E−04 | 0.93 | 1.04 | 58 | MAA and Josa vs. Clari |
| 49710 | WSX1 | | class I cytokine receptor | 9.25E−04 | 0.94 | 1.06 | 58 | MAA and Josa vs. Clari |
| 47761 | | | | 9.51E−04 | 1.14 | 1.02 | 56 | MAA and Josa vs. Clari |
| 48557 | NFE2L1 | NFE2L1 | nuclear factor (erythroid-derived 2)-like 1 | 9.65E−04 | 0.97 | 1.07 | 57 | MAA and Josa vs. Clari |
| 50424 | HTGN29 | | HTGN29 protein | 9.65E−04 | 0.92 | 1.02 | 57 | MAA and Josa vs. Clari |
| 49420 | TIMP2 | TIMP2 | tissue inhibitor of metalloproteinase 2 | 9.77E−04 | 1.09 | 1.40 | 58 | MAA and Josa vs. Clari |
| 49869 | ARG2 | | arginase, type II | 9.78E−04 | 1.09 | 0.93 | 52 | MAA and Josa vs. Clari |

Genes statistically predictive of Clari and Roxi response
Genes differentially expressed in response to Clari and Roxi but not the inactive macrolides MAA or Josa

| SpotID | GeneSymbol | GeneAssignment | p value | mean group 1 | mean group 2 | # expts | ClusterRString1 |
|---|---|---|---|---|---|---|---|
| 50639 | PDIR | for protein disulfide isomerase-related | 3.22E−09 | 0.91 | 1.13 | 71 | MAA and Josa vs. Clari, Roxi |
| 50654 | COPB2 | coatomer protein complex, subunit beta 2 (beta | 3.57E−09 | 0.99 | 1.15 | 72 | MAA and Josa vs. Clari, Roxi |
| | | prime) | | | | | |
| 49380 | SLC35A2 | solute carrier family 35 (UDP-galactose | 9.46E−09 | 0.99 | 1.12 | 72 | MAA and Josa vs. Clari, Roxi |
| | | transporter), member 2 | | | | | |
| 47705 | MAD4 | Mad4 homolog | 3.41E−08 | 0.97 | 1.29 | 72 | MAA and Josa vs. Clari, Roxi |
| 50302 | CDR2 | cerebellar degeneration-related protein (62 kD) | 4.43E−08 | 0.89 | 1.10 | 70 | MAA and Josa vs. Clari, Roxi |
| 47249 | AKR1A1 | aldo-keto reductase family 1, member A1 | 1.33E−07 | 0.93 | 1.09 | 72 | MAA and Josa vs. Clari, Roxi |
| | | (aldehyde reductase) | | | | | |
| 47131 | KIAA0251 | KIAA0251 protein | 1.95E−07 | 0.92 | 1.10 | 71 | MAA and Josa vs. Clari, Roxi |
| 50332 | DAD1 | defender against cell death 1 | 2.06E−07 | 1.02 | 1.21 | 72 | MAA and Josa vs. Clari, Roxi |
| 50084 | IDI1 | isopentenyl-diphosphate delta isomerase | 2.20E−07 | 0.90 | 1.24 | 72 | MAA and Josa vs. Clari, Roxi |
| 48470 | MCM5 | MCM5 minichromosome maintenance deficient 5, | 2.34E−07 | 0.97 | 0.73 | 72 | MAA and Josa vs. Clari, Roxi |
| | | cell division cycle 46 (S. cerevisiae) | | | | | |
| 47251 | HKE4 | HLA class II region expressed gene KE4 | 2.69E−07 | 0.94 | 1.15 | 71 | MAA and Josa vs. Clari, Roxi |
| 47755 | KIAA1922 | KIAA1922 protein | 3.71E−07 | 1.14 | 0.84 | 56 | MAA and Josa vs. Clari, Roxi |
| 49922 | DHCR24 | 24-dehydrocholesterol reductase | 4.07E−07 | 1.00 | 1.21 | 72 | MAA and Josa vs. Clari, Roxi |
| 49705 | PCYT2 | phosphate cytidylyltransferase 2, ethanolamine | 5.19E−07 | 0.92 | 1.19 | 72 | MAA and Josa vs. Clari, Roxi |
| 50307 | MAX | MAX protein | 5.86E−07 | 0.96 | 1.10 | 72 | MAA and Josa vs. Clari, Roxi |
| 49512 | GC20 | translation factor sui1 homolog | 6.61E−07 | 1.01 | 1.18 | 72 | MAA and Josa vs. Clari, Roxi |
| 49735 | GSK3B | glycogen synthase kinase 3 beta | 7.91E−07 | 1.02 | 1.12 | 72 | MAA and Josa vs. Clari, Roxi |
| 47926 | CCNE2 | cyclin E2 | 1.32E−06 | 0.98 | 0.74 | 56 | MAA and Josa vs. Clari, Roxi |
| 50659 | MAX | MAX protein | 1.80E−06 | 0.95 | 1.09 | 72 | MAA and Josa vs. Clari, Roxi |
| 47212 | ITGB5 | integrin, beta 5 | 2.57E−06 | 0.94 | 1.13 | 71 | MAA and Josa vs. Clari, Roxi |
| 48998 | FADS1 | fatty acid desaturase 1 | 2.84E−06 | 1.02 | 1.35 | 72 | MAA and Josa vs. Clari, Roxi |
| 50337 | FGFR2 | fibroblast growth factor receptor 2 (bacteria- | 3.75E−06 | 0.93 | 1.07 | 66 | MAA and Josa vs. Clari, Roxi |
| | | expressed kinase, keratinocyte growth factor | | | | | |
| | | receptor, craniofacial dysostosis 1, Crouzon | | | | | |
| | | syndrome, Pfeiffer syndrome, Jackson-Weiss | | | | | |
| | | syndrome) | | | | | |
| 50135 | RRBP1 | ribosome binding protein 1 homolog 180 kD (dog) | 4.46E−06 | 0.95 | 1.11 | 72 | MAA and Josa vs. Clari, Roxi |
| 50412 | AGPAT1 | 1-acylglycerol-3-phosphate O-acyltransferase 1 | 4.72E−06 | 0.99 | 1.10 | 72 | MAA and Josa vs. Clari, Roxi |
| | | (lysophosphatidic acid acyltransferase, alpha) | | | | | |
| 47949 | BCKDK | branched chain alpha-ketoacid dehydrogenase | 5.88E−06 | 1.07 | 0.95 | 72 | MAA and Josa vs. Clari, Roxi |
| | | kinase | | | | | |
| 48971 | P4HA2 | procollagen-proline, 2-oxoglutarate 4-dioxygenase | 6.08E−06 | 1.07 | 1.41 | 71 | MAA and Josa vs. Clari, Roxi |
| | | (proline 4-hydroxylase), alpha polypeptide II | | | | | |
| 50550 | PTPRT | protein tyrosine phosphatase, receptor type, T | 6.81E−06 | 0.92 | 1.05 | 71 | MAA and Josa vs. Clari, Roxi |
| 47123 | MCM2 | MCM2 minichromosome maintenance deficient 2, | 6.92E−06 | 0.97 | 0.81 | 68 | MAA and Josa vs. Clari, Roxi |
| | | mitotin (S. cerevisiae) | | | | | |
| 48397 | HIF1A | hypoxia-inducible factor 1, alpha subunit (basic | 9.10E−06 | 1.09 | 0.92 | 72 | MAA and Josa vs. Clari, Roxi |
| | | helix-loop-helix transcription factor) | | | | | |
| 48807 | UNG | uracil-DNA glycosylase | 9.61E−06 | 1.03 | 0.92 | 72 | MAA and Josa vs. Clari, Roxi |
| 49163 | LGALS3 | lectin, galactoside-binding, soluble, 3 (galectin 3) | 9.61E−06 | 1.06 | 1.21 | 72 | MAA and Josa vs. Clari, Roxi |
| 49144 | PIK3R3 | phosphoinositide-3-kinase, regulatory subunit, | 1.07E−05 | 1.01 | 1.13 | 72 | MAA and Josa vs. Clari, Roxi |
| | | polypeptide 3 (p55, gamma) | | | | | |
| 47951 | COX15 | COX15 homolog, cytochrome c oxidase assembly | 1.07E−05 | 1.05 | 0.95 | 72 | MAA and Josa vs. Clari, Roxi |
| | | protein (yeast) | | | | | |
| 47181 | RAB2 | RAB2, member RAS oncogene family | 1.32E−05 | 1.01 | 1.12 | 72 | MAA and Josa vs. Clari, Roxi |
| 48328 | FN1 | fibronectin 1 | 1.32E−05 | 1.00 | 0.62 | 72 | MAA and Josa vs. Clari, Roxi |
| 49347 | HNRPA3 | heterogeneous nuclear ribonucleoprotein A3 | 1.48E−05 | 1.01 | 0.78 | 69 | MAA and Josa vs. Clari, Roxi |
| 48508 | USP24 | ubiquitin specific protease 24 | 1.55E−05 | 1.03 | 0.90 | 72 | MAA and Josa vs. Clari, Roxi |
| 49688 | GSK3B | glycogen synthase kinase 3 beta | 1.64E−05 | 1.05 | 1.20 | 72 | MAA and Josa vs. Clari, Roxi |
| 49757 | DDOST | dolichyl-diphosphooligosaccharide-protein | 1.72E−05 | 0.96 | 1.08 | 72 | MAA and Josa vs. Clari, Roxi |
| | | glycosyltransferase | | | | | |
| 48713 | MKNK2 | G protein-coupled receptor kinase 7 | 1.72E−05 | 0.96 | 1.17 | 72 | MAA and Josa vs. Clari, Roxi |
| 49177 | NEDD8 | neural precursor cell expressed, developmentally | 2.24E−05 | 0.97 | 1.06 | 72 | MAA and Josa vs. Clari, Roxi |
| | | down-regulated 8 | | | | | |
| 49989 | VTN | vitronectin (serum spreading factor, somatomedin | 2.71E−05 | 1.19 | 0.94 | 57 | MAA and Josa vs. Clari, Roxi |
| | | B, complement S-protein) | | | | | |
| 46851 | RRBP1 | ribosome binding protein 1 homolog 180 kD (dog) | 2.75E−05 | 0.97 | 1.09 | 72 | MAA and Josa vs. Clari, Roxi |
| 49511 | MATN2 | matrilin 2 | 3.04E−05 | 0.93 | 1.11 | 69 | MAA and Josa vs. Clari, Roxi |
| 49715 | HNRPD | heterogeneous nuclear ribonucleoprotein D (AU- | 3.05E−05 | 0.99 | 0.91 | 72 | MAA and Josa vs. Clari, Roxi |
| | | rich element RNA binding protein 1, 37 kD) | | | | | |
| 48451 | M6PR | mannose-6-phosphate receptor (cation | 3.21E−05 | 0.97 | 1.08 | 72 | MAA and Josa vs. Clari, Roxi |
| | | dependent) | | | | | |
| 46882 | HEXA | hexosaminidase A (alpha polypeptide) | 3.37E−05 | 0.97 | 1.11 | 72 | MAA and Josa vs. Clari, Roxi |
| 48348 | FN1 | fibronectin 1 | 3.55E−05 | 1.00 | 0.62 | 72 | MAA and Josa vs. Clari, Roxi |
| 50222 | FLJ13868 | hypothetical protein FLJ13868 | 3.55E−05 | 0.93 | 1.07 | 72 | MAA and Josa vs. Clari, Roxi |
| 48644 | KIAA0323 | KIAA0323 protein | 3.55E−05 | 1.01 | 1.11 | 72 | MAA and Josa vs. Clari, Roxi |
| 49873 | INSIG1 | insulin induced gene 1 | 3.55E−05 | 0.93 | 1.37 | 72 | MAA and Josa vs. Clari, Roxi |
| 49967 | CCNE2 | cyclin E2 | 3.71E−05 | 1.02 | 0.81 | 58 | MAA and Josa vs. Clari, Roxi |
| 47947 | SF3B3 | splicing factor 3b, subunit 3, 130 kD | 3.73E−05 | 1.10 | 0.96 | 72 | MAA and Josa vs. Clari, Roxi |
| 46991 | TFPI2 | tissue factor pathway inhibitor 2 | 4.51E−05 | 1.00 | 0.66 | 68 | MAA and Josa vs. Clari, Roxi |
| 48952 | ELK1 | ELK1, member of ETS oncogene family | 5.04E−05 | 0.98 | 1.07 | 72 | MAA and Josa vs. Clari, Roxi |
| 49047 | GLUD1 | glutamate dehydrogenase 1 | 5.85E−05 | 1.05 | 0.95 | 72 | MAA and Josa vs. Clari, Roxi |
| 49352 | NCBP2 | nuclear cap binding protein subunit 2, 20 kD | 6.38E−05 | 0.94 | 1.02 | 71 | MAA and Josa vs. Clari, Roxi |
| 50405 | CHERP | calcium homeostasis endoplasmic reticulum | 6.46E−05 | 1.00 | 0.90 | 72 | MAA and Josa vs. Clari, Roxi |
| | | protein | | | | | |
| 47661 | HSPA9B | heat shock 70 kD protein 9B (mortalin-2) | 6.46E−05 | 1.10 | 0.95 | 72 | MAA and Josa vs. Clari, Roxi |
| 49761 | BCL2L2 | BCL2-like 2 | 6.71E−05 | 0.85 | 0.99 | 71 | MAA and Josa vs. Clari, Roxi |
| 49649 | FGF2 | fibroblast growth factor 2 (basic) | 7.04E−05 | 1.12 | 0.81 | 51 | MAA and Josa vs. Clari, Roxi |
| 47406 | MTHFD1 | methylenetetrahydrofolate dehydrogenase | 7.12E−05 | 1.12 | 0.95 | 72 | MAA and Josa vs. Clari, Roxi |
| | | (NADP+ dependent), methenyltetrahydrofolate | | | | | |
| | | cyclohydrolase, formyltetrahydrofolate synthetase | | | | | |
| 47192 | ARHGAP1 | Rho GTPase activating protein 1 | 7.12E−05 | 1.10 | 0.96 | 72 | MAA and Josa vs. Clari, Roxi |
| 50284 | UQCR | ubiquinol-cytochrome c reductase (6.4 kD) subunit | 7.85E−05 | 0.83 | 0.98 | 72 | MAA and Josa vs. Clari, Roxi |
| 49650 | MAP3K10 | mitogen-activated protein kinase kinase kinase 10 | 7.85E−05 | 0.93 | 1.10 | 72 | MAA and Josa vs. Clari, Roxi |
| 47535 | ARHGDIA | Rho GDP dissociation inhibitor (GDI) alpha | 7.85E−05 | 1.08 | 0.93 | 72 | MAA and Josa vs. Clari, Roxi |
| 50224 | HK2 | hexokinase 2 | 8.24E−05 | 0.95 | 1.11 | 72 | MAA and Josa vs. Clari, Roxi |
| 48333 | DNMT1 | DNA (cytosine-5-)-methyltransferase 1 | 8.61E−05 | 0.97 | 0.84 | 71 | MAA and Josa vs. Clari, Roxi |
| 48643 | DIP2 | KIAA0184 protein | 9.08E−05 | 1.00 | 0.89 | 72 | MAA and Josa vs. Clari, Roxi |
| 50103 | IRF5 | interferon regulatory factors | 9.08E−05 | 0.87 | 0.99 | 72 | MAA and Josa vs. Clari, Roxi |
| 48006 | BCL2L2 | BCL2-like 2 | 9.52E−05 | 0.86 | 0.99 | 72 | MAA and Josa vs. Clari, Roxi |
| 49990 | SRC | v-src sarcoma (Schmidt-Ruppin A-2) viral | 9.98E−05 | 0.99 | 1.13 | 70 | MAA and Josa vs. Clari, Roxi |
| | | oncogene homolog (avian) | | | | | |
| 47939 | INPP5E | KIAA0123 protein | 9.99E−05 | 0.99 | 0.91 | 72 | MAA and Josa vs. Clari, Roxi |
| 48192 | OPA1 | optic atrophy 1 (autosomal dominant) | 9.99E−05 | 1.02 | 0.92 | 72 | MAA and Josa vs. Clari, Roxi |
| 49481 | LOC55871 | COBW-like protein | 9.99E−05 | 0.90 | 1.01 | 72 | MAA and Josa vs. Clari, Roxi |
| 49608 | POU4F1 | POU domain, class 4, transcription factor 1 | 9.99E−05 | 0.97 | 1.04 | 72 | MAA and Josa vs. Clari, Roxi |
| 49142 | PPIB | peptidylprolyl isomerase B (cyclophilin B) | 9.99E−05 | 1.03 | 1.16 | 72 | MAA and Josa vs. Clari, Roxi |
| 49032 | GSK3B | glycogen synthase kinase 3 beta | 1.05E−04 | 1.06 | 1.18 | 72 | MAA and Josa vs. Clari, Roxi |
| 48502 | CLIC4 | chloride intracellular channel 4 | 1.08E−04 | 1.17 | 1.00 | 59 | MAA and Josa vs. Clari, Roxi |
| 50228 | MVD | mevalonate (diphospho) decarboxylase | 1.10E−04 | 0.92 | 1.17 | 72 | MAA and Josa vs. Clari, Roxi |
| 50319 | BMP6 | bone morphogenetic protein 6 | 1.10E−04 | 0.98 | 1.11 | 70 | MAA and Josa vs. Clari, Roxi |
| 50216 | | | 1.10E−04 | 0.95 | 1.04 | 71 | MAA and Josa vs. Clari, Roxi |
| 48672 | CHK | choline kinase | 1.15E−04 | 0.87 | 1.00 | 72 | MAA and Josa vs. Clari, Roxi |
| 49183 | SRRM1 | serine/arginine repetitive matrix 1 | 1.15E−04 | 0.96 | 0.85 | 72 | MAA and Josa vs. Clari, Roxi |
| 47793 | BET3 | similar to yeast BET3 (S. cerevisiae) | 1.21E−04 | 0.95 | 1.06 | 72 | MAA and Josa vs. Clari, Roxi |
| 47995 | PTGER1 | prostaglandin E receptor 1 (subtype EP1), 42 kD | 1.27E−04 | 1.00 | 0.77 | 53 | MAA and Josa vs. Clari, Roxi |
| 49172 | YAF2 | YY1 associated factor 2 | 1.28E−04 | 1.01 | 0.92 | 55 | MAA and Josa vs. Clari, Roxi |
| 47111 | GOLGB1 | golgi autoantigen, golgin subfamily b, macrogolgin | 1.28E−04 | 1.01 | 1.14 | 70 | MAA and Josa vs. Clari, Roxi |
| | | (with transmembrane signal), 1 | | | | | |
| 49013 | BBC3 | Bcl-2 binding component 3 | 1.33E−04 | 0.93 | 1.04 | 72 | MAA and Josa vs. Clari, Roxi |
| 46579 | SERPING1 | serine (or cysteine) proteinase inhibitor, clade G | 1.33E−04 | 1.03 | 0.88 | 69 | MAA and Josa vs. Clari, Roxi |
| | | (C1 inhibitor), member 1, (angioedema, hereditary) | | | | | |
| 49794 | BCL2L2 | BCL2-like 2 | 1.34E−04 | 0.86 | 1.00 | 71 | MAA and Josa vs. Clari, Roxi |
| 50464 | MMP15 | matrix metalloproteinase 15 (membrane-inserted) | 1.35E−04 | 0.87 | 1.04 | 69 | MAA and Josa vs. Clari, Roxi |
| 49781 | P4HB | procollagen-proline, 2-oxoglutarate 4-dioxygenase | 1.39E−04 | 0.98 | 1.10 | 72 | MAA and Josa vs. Clari, Roxi |
| | | (proline 4-hydroxylase), beta polypeptide (protein | | | | | |
| | | disulfide isomerase; thyroid hormone binding | | | | | |
| | | protein p55) | | | | | |
| 50099 | CNOT3 | CCR4-NOT transcription complex, subunit 3 | 1.39E−04 | 0.95 | 0.86 | 72 | MAA and Josa vs. Clari, Roxi |
| 49360 | TEB4 | similar to S. cerevisiae SSM4 | 1.46E−04 | 1.10 | 0.94 | 72 | MAA and Josa vs. Clari, Roxi |
| 48725 | IKKE | IKK-related kinase epsilon; inducible IkappaB | 1.46E−04 | 0.84 | 0.94 | 72 | MAA and Josa vs. Clari, Roxi |
| | | kinase | | | | | |
| 49514 | UBC | ubiquitin C | 1.53E−04 | 0.97 | 1.09 | 72 | MAA and Josa vs. Clari, Roxi |
| 48530 | APLP2 | amyloid beta (A4) precursor-like protein 2 | 1.53E−04 | 0.95 | 1.10 | 72 | MAA and Josa vs. Clari, Roxi |
| 50421 | GOCAP1 | golgi phosphoprotein 1 | 1.55E−04 | 1.01 | 1.10 | 71 | MAA and Josa vs. Clari, Roxi |
| 50022 | HIF1A | hypoxia-inducible factor 1, alpha subunit (basic | 1.60E−04 | 1.07 | 0.93 | 72 | MAA and Josa vs. Clari, Roxi |
| | | helix-loop-helix transcription factor) | | | | | |
| 47987 | THBS1 | thrombospondin 1 | 1.72E−04 | 1.02 | 0.71 | 60 | MAA and Josa vs. Clari, Roxi |
| 46679 | KIAA1172 | pre-mRNA splicing SR protein rA4 | 1.73E−04 | 1.04 | 0.92 | 70 | MAA and Josa vs. Clari, Roxi |
| 49446 | SRC | v-src sarcoma (Schmidt-Ruppin A-2) viral | 1.74E−04 | 1.00 | 1.14 | 71 | MAA and Josa vs. Clari, Roxi |
| | | oncogene homolog (avian) | | | | | |
| 49006 | PRSS25 | protease, serine, 25 | 1.76E−04 | 1.02 | 1.14 | 72 | MAA and Josa vs. Clari, Roxi |
| 48707 | P4HA1 | procollagen-proline, 2-oxoglutarate 4-dioxygenase | 1.76E−04 | 0.97 | 1.18 | 72 | MAA and Josa vs. Clari, Roxi |
| | | (proline 4-hydroxylase), alpha polypeptide I | | | | | |
| 48620 | XBP1 | X-box binding protein 1 | 1.84E−04 | 0.98 | 1.10 | 72 | MAA and Josa vs. Clari, Roxi |
| 48921 | ITGAE | integrin, alpha E (antigen CD103, human mucosal | 1.87E−04 | 0.85 | 0.97 | 71 | MAA and Josa vs. Clari, Roxi |
| | | lymphocyte antigen 1; alpha polypeptide) | | | | | |
| 47776 | POLA | polymerase (DNA directed), alpha | 1.88E−04 | 1.02 | 0.89 | 59 | MAA and Josa vs. Clari, Roxi |
| 48456 | DDC | dopa decarboxylase (aromatic L-amino acid | 1.91E−04 | 1.12 | 0.97 | 59 | MAA and Josa vs. Clari, Roxi |
| | | decarboxylase) | | | | | |
| 47846 | SSI-1 | JAK binding protein | 1.99E−04 | 1.03 | 0.93 | 69 | MAA and Josa vs. Clari, Roxi |
| 49314 | PPIB | peptidylprolyl isomerase B (cyclophilin B) | 2.12E−04 | 1.04 | 1.16 | 72 | MAA and Josa vs. Clari, Roxi |
| 47935 | P4HA1 | procollagen-proline, 2-oxoglutarate 4-dioxygenase | 2.30E−04 | 0.95 | 1.18 | 70 | MAA and Josa vs. Clari, Roxi |
| | | (proline 4-hydroxylase), alpha polypeptide I | | | | | |
| 50736 | PRKDC | protein kinase, DNA-activated, catalytic | 2.32E−04 | 1.07 | 0.92 | 72 | MAA and Josa vs. Clari, Roxi |
| | | polypeptide | | | | | |
| 47811 | ENTPD6 | ectonucleoside triphosphate diphosphohydrolase 6 | 2.32E−04 | 0.93 | 1.06 | 72 | MAA and Josa vs. Clari, Roxi |
| | | (putative function | | | | | |
| 47622 | TRAP1 | heat shock protein 75 | 2.43E−04 | 1.09 | 0.94 | 72 | MAA and Josa vs. Clari, Roxi |
| 48486 | | | 2.45E−04 | 1.21 | 0.94 | 34 | MAA and Josa vs. Clari, Roxi |
| 50738 | AD022 | TRAF and TNF receptor-associated protein | 2.53E−04 | 0.94 | 1.10 | 70 | MAA and Josa vs. Clari, Roxi |
| 46825 | MKNK2 | G protein-coupled receptor kinase 7 | 2.54E−04 | 0.94 | 1.12 | 72 | MAA and Josa vs. Clari, Roxi |
| 49169 | RRS1 | homolog of yeast ribosome biogenesis regulatory | 2.55E−04 | 1.09 | 0.93 | 68 | MAA and Josa vs. Clari, Roxi |
| | | protein RRS1 | | | | | |
| 50590 | CKS2 | CDC28 protein kinase 2 | 2.59E−04 | 0.74 | 0.89 | 69 | MAA and Josa vs. Clari, Roxi |
| 50050 | NDUFA2 | NADH dehydrogenase (ubiquinone) 1 alpha | 2.66E−04 | 0.90 | 1.02 | 72 | MAA and Josa vs. Clari, Roxi |
| | | subcomplex, 2 (8 kD, B8) | | | | | |
| 50342 | IKKE | IKK-related kinase epsilon; inducible IkappaB | 2.79E−04 | 0.94 | 1.08 | 70 | MAA and Josa vs. Clari, Roxi |
| | | kinase | | | | | |
| 47522 | SERPINA1 | serine (or cysteine) proteinase inhibitor, clade A | 2.85E−04 | 1.04 | 0.95 | 71 | MAA and Josa vs. Clari, Roxi |
| | | (alpha-1 antiproteinase, antitrypsin), member 1 | | | | | |
| 50645 | BET1 | BET1 homolog (S. cerevisiae) | 2.92E−04 | 0.84 | 1.01 | 70 | MAA and Josa vs. Clari, Roxi |
| 48717 | PPP3CB | protein phosphatase 3 (formerly 2B), catalytic | 2.99E−04 | 1.09 | 0.95 | 52 | MAA and Josa vs. Clari, Roxi |
| | | subunit, beta isoform (calcineurin A beta) | | | | | |
| 47950 | DNASE1L1 | deoxyribonuclease I-like 1 | 3.04E−04 | 0.96 | 1.03 | 72 | MAA and Josa vs. Clari, Roxi |
| 48557 | NFE2L1 | nuclear factor (erythroid-derived 2)-like 1 | 3.13E−04 | 0.97 | 1.06 | 71 | MAA and Josa vs. Clari, Roxi |
| 48026 | SRF | serum response factor (c-fos serum response | 3.18E−04 | 0.94 | 1.03 | 72 | MAA and Josa vs. Clari, Roxi |
| | | element-binding transcription factor) | | | | | |
| 49374 | | | 3.18E−04 | 0.97 | 1.03 | 72 | MAA and Josa vs. Clari, Roxi |
| 49007 | TM9SF2 | transmembrane 9 superfamily member 2 | 3.18E−04 | 1.08 | 1.18 | 72 | MAA and Josa vs. Clari, Roxi |
| 49737 | CHAF1B | chromatin assembly factor 1, subunit B (p60) | 3.23E−04 | 0.99 | 0.88 | 66 | MAA and Josa vs. Clari, Roxi |
| 46935 | METAP2 | methionyl aminopeptidase 2 | 3.33E−04 | 1.05 | 0.89 | 72 | MAA and Josa vs. Clari, Roxi |
| 46843 | ARHGDIA | Rho GDP dissociation inhibitor (GDI) alpha | 3.33E−04 | 1.08 | 0.93 | 72 | MAA and Josa vs. Clari, Roxi |
| 48804 | KIAA0217 | KIAA0217 protein | 3.37E−04 | 1.01 | 0.88 | 70 | MAA and Josa vs. Clari, Roxi |
| 47439 | DCTN1 | dynactin 1 (p150, glued homolog, Drosophila) | 3.48E−04 | 1.04 | 0.95 | 72 | MAA and Josa vs. Clari, Roxi |
| 47418 | SNK | serum-inducible kinase | 3.53E−04 | 1.03 | 0.82 | 70 | MAA and Josa vs. Clari, Roxi |
| 48137 | FRDA | Friedreich ataxia | 3.53E−04 | 1.03 | 0.90 | 70 | MAA and Josa vs. Clari, Roxi |
| 48693 | HSPBP1 | hsp70-interacting protein | 3.64E−04 | 1.04 | 0.91 | 72 | MAA and Josa vs. Clari, Roxi |
| 49155 | ARTS-1 | type 1 tumor necrosis factor receptor shedding | 3.65E−04 | 1.11 | 0.86 | 61 | MAA and Josa vs. Clari, Roxi |
| | | aminopeptidase regulator | | | | | |
| 49490 | VTN | vitronectin (serum spreading factor, somatomedin | 3.76E−04 | 1.12 | 0.94 | 54 | MAA and Josa vs. Clari, Roxi |
| | | B, complement S-protein) | | | | | |
| 46896 | CDIPT | CDP-diacylglycerol--inositol 3- | 3.80E−04 | 0.91 | 1.01 | 72 | MAA and Josa vs. Clari, Roxi |
| | | phosphatidyltransferase (phosphatidylinositol | | | | | |
| | | synthase) | | | | | |
| 48301 | GCN5L1 | GCN5 general control of amino-acid synthesis 5- | 3.80E−04 | 0.93 | 1.01 | 72 | MAA and Josa vs. Clari, Roxi |
| | | like 1 (yeast) | | | | | |
| 49577 | LOC54543 | 6.2 kd protein | 3.80E−04 | 0.88 | 1.02 | 72 | MAA and Josa vs. Clari, Roxi |
| 50267 | IL10RB | interleukin 10 receptor, beta | 3.80E−04 | 0.98 | 1.08 | 72 | MAA and Josa vs. Clari, Roxi |
| 48233 | MAPK12 | mitogen-activated protein kinase 12 | 3.97E−04 | 0.88 | 1.03 | 72 | MAA and Josa vs. Clari, Roxi |
| 50584 | BN51T | BN51 (BHK21) temperature sensitivity | 3.97E−04 | 0.97 | 1.07 | 72 | MAA and Josa vs. Clari, Roxi |
| | | complementing | | | | | |
| 47241 | NUBP1 | nucleotide binding protein 1 (MinD homolog, E. coli) | 4.11E−04 | 1.04 | 0.95 | 69 | MAA and Josa vs. Clari, Roxi |
| 47366 | P4HB | procollagen-proline, 2-oxoglutarate 4-dioxygenase | 4.15E−04 | 1.05 | 1.18 | 72 | MAA and Josa vs. Clari, Roxi |
| | | (proline 4-hydroxylase), beta polypeptide (protein | | | | | |
| | | disulfide isomerase; thyroid hormone binding | | | | | |
| | | protein p55) | | | | | |
| 48160 | ZAP3 | ZAP3 protein | 4.27E−04 | 1.08 | 0.95 | 66 | MAA and Josa vs. Clari, Roxi |
| 48415 | SERPINB1 | serine (or cysteine) proteinase inhibitor, clade B | 4.45E−04 | 1.02 | 1.28 | 45 | MAA and Josa vs. Clari, Roxi |
| | | (ovalbumin), member 1 | | | | | |
| 48521 | ARAF1 | v-raf murine sarcoma 3611 viral oncogene | 4.53E−04 | 1.12 | 0.98 | 72 | MAA and Josa vs. Clari, Roxi |
| | | homolog 1 | | | | | |
| 49233 | APOE | apolipoprotein E | 4.53E−04 | 0.96 | 1.01 | 72 | MAA and Josa vs. Clari, Roxi |
| 50591 | | | 4.53E−04 | 1.01 | 1.13 | 72 | MAA and Josa vs. Clari, Roxi |
| 49435 | TRRAP | transformation/transcription domain-associated | 4.74E−04 | 1.07 | 0.92 | 72 | MAA and Josa vs. Clari, Roxi |
| | | protein | | | | | |
| 47504 | MSH3 | mutS homolog 3 (E. coli) | 4.91E−04 | 1.09 | 0.99 | 54 | MAA and Josa vs. Clari, Roxi |
| 48500 | RPL13A | ribosomal protein L13a | 4.95E−04 | 1.05 | 0.92 | 72 | MAA and Josa vs. Clari, Roxi |
| 48266 | GRB2 | growth factor receptor-bound protein 2 | 4.95E−04 | 0.93 | 1.04 | 72 | MAA and Josa vs. Clari, Roxi |
| 49767 | AD022 | TRAF and TNF receptor-associated protein | 5.14E−04 | 0.96 | 1.10 | 71 | MAA and Josa vs. Clari, Roxi |
| 49524 | AP1G2 | adaptor-related protein complex 1, gamma 2 | 5.17E−04 | 0.74 | 0.92 | 72 | MAA and Josa vs. Clari, Roxi |
| | | subunit | | | | | |
| 47432 | GCS1 | glucosidase I | 5.17E−04 | 0.90 | 1.02 | 72 | MAA and Josa vs. Clari, Roxi |
| 49915 | ATP2B4 | ATPase, Ca++ transporting, plasma membrane 4 | 5.17E−04 | 1.23 | 1.06 | 72 | MAA and Josa vs. Clari, Roxi |
| 47314 | EPOR | erythropoietin receptor | 5.19E−04 | 0.99 | 1.14 | 58 | MAA and Josa vs. Clari, Roxi |
| 46633 | FLJ20699 | hypothetical protein FLJ20699 | 5.23E−04 | 1.00 | 0.81 | 39 | MAA and Josa vs. Clari, Roxi |
| 49679 | SERPINF1 | serine (or cysteine) proteinase inhibitor, clade F | 5.24E−04 | 0.98 | 1.09 | 65 | MAA and Josa vs. Clari, Roxi |
| | | (alpha-2 antiplasmin, pigment epithelium derived | | | | | |
| | | factor), member 1 | | | | | |
| 49117 | SERPINB1 | serine (or cysteine) proteinase inhibitor, clade B | 5.39E−04 | 1.01 | 0.88 | 72 | MAA and Josa vs. Clari, Roxi |
| | | (ovalbumin), member 1 | | | | | |
| 46988 | EREG | epiregulin | 5.40E−04 | 1.11 | 0.85 | 48 | MAA and Josa vs. Clari, Roxi |
| 49673 | BTN3A2 | butyrophilin, subfamily 3, member A2 | 5.60E−04 | 0.92 | 1.11 | 59 | MAA and Josa vs. Clari, Roxi |
| 47034 | C2orf3 | chromosome 2 open reading frame 3 | 5.69E−04 | 1.01 | 0.90 | 63 | MAA and Josa vs. Clari, Roxi |
| 48149 | SFRS7 | splicing factor, arginine/serine-rich 7 (35 kD) | 5.70E−04 | 0.98 | 0.88 | 69 | MAA and Josa vs. Clari, Roxi |
| 47850 | PRLR | prolactin receptor | 5.74E−04 | 1.09 | 0.99 | 66 | MAA and Josa vs. Clari, Roxi |
| 47679 | NR4A2 | nuclear receptor subfamily 4, group A, member 2 | 5.80E−04 | 0.95 | 1.14 | 63 | MAA and Josa vs. Clari, Roxi |
| 47415 | CETN2 | centrin, EF-hand protein, 2 | 5.88E−04 | 1.06 | 1.18 | 72 | MAA and Josa vs. Clari, Roxi |
| 48747 | MAFB | v-maf musculoaponeurotic fibrosarcoma oncogene | 5.88E−04 | 1.14 | 0.93 | 25 | MAA and Josa vs. Clari, Roxi |
| | | homolog B (avian) | | | | | |
| 49028 | BNIP3L | BCL2/adenovirus E1B 19 kD interacting protein 3- | 6.14E−04 | 1.03 | 1.20 | 72 | MAA and Josa vs. Clari, Roxi |
| | | like | | | | | |
| 50349 | LOC51295 | ECSIT | 6.25E−04 | 1.01 | 0.84 | 69 | MAA and Josa vs. Clari, Roxi |
| 47802 | GOT1 | glutamic-oxaloacetic transaminase 1, soluble | 6.40E−04 | 1.02 | 0.94 | 69 | MAA and Josa vs. Clari, Roxi |
| | | (aspartate aminotransferase 1) | | | | | |
| 50513 | ARHC | ras homolog gene family, member C | 6.68E−04 | 0.91 | 1.00 | 72 | MAA and Josa vs. Clari, Roxi |
| 50079 | SCOP | SCN Circadian Oscillatory Protein (SCOP) | 6.68E−04 | 0.97 | 1.04 | 72 | MAA and Josa vs. Clari, Roxi |
| 50511 | MGP | matrix Gla protein | 6.85E−04 | 0.83 | 0.98 | 33 | MAA and Josa vs. Clari, Roxi |
| 48646 | SDCBP | syndecan binding protein (syntenin) | 6.97E−04 | 0.97 | 1.08 | 72 | MAA and Josa vs. Clari, Roxi |
| 49272 | TNFRSF10B | tumor necrosis factor receptor superfamily, | 6.97E−04 | 0.97 | 1.08 | 72 | MAA and Josa vs. Clari, Roxi |
| | | member 10b | | | | | |
| 49800 | AD022 | TRAF and TNF receptor-associated protein | 7.00E−04 | 0.97 | 1.09 | 71 | MAA and Josa vs. Clari, Roxi |
| 48411 | MGEA5 | meningioma expressed antigen 5 (hyaluronidase) | 7.00E−04 | 0.97 | 1.13 | 71 | MAA and Josa vs. Clari, Roxi |
| 50601 | SRP19 | signal recognition particle 19 kD | 7.01E−04 | 0.95 | 1.06 | 70 | MAA and Josa vs. Clari, Roxi |
| 48004 | CUTL1 | cut-like 1, CCAAT displacement protein | 7.07E−04 | 1.22 | 1.02 | 67 | MAA and Josa vs. Clari, Roxi |
| | | (Drosophila) | | | | | |
| 49318 | YWHAH | tyrosine 3-monooxygenase/tryptophan 5- | 7.28E−04 | 1.08 | 0.97 | 72 | MAA and Josa vs. Clari, Roxi |
| | | monooxygenase activation protein, eta polypeptide | | | | | |
| 48461 | BARD1 | BRCA1 associated RING domain 1 | 7.50E−04 | 0.98 | 0.86 | 51 | MAA and Josa vs. Clari, Roxi |
| 49618 | CLN2 | ceroid-lipofuscinosis, neuronal 2, late infantile | 7.59E−04 | 1.03 | 1.12 | 72 | MAA and Josa vs. Clari, Roxi |
| | | (Jansky-Bielschowsky disease) | | | | | |
| 48345 | CCT3 | chaperonin containing TCP1, subunit 3 (gamma) | 7.92E−04 | 0.97 | 1.03 | 72 | MAA and Josa vs. Clari, Roxi |
| 48365 | MAPK9 | mitogen-activated protein kinase 9 | 7.92E−04 | 1.04 | 0.94 | 72 | MAA and Josa vs. Clari, Roxi |
| 49606 | CSTF3 | cleavage stimulation factor, 3′ pre-RNA, subunit 3, | 7.97E−04 | 0.98 | 1.07 | 71 | MAA and Josa vs. Clari, Roxi |
| | | 77 kD | | | | | |
| 48654 | FECH | ferrochelatase (protoporphyria) | 8.26E−04 | 1.05 | 0.98 | 72 | MAA and Josa vs. Clari, Roxi |
| 47754 | NBS1 | Nijmegen breakage syndrome 1 (nibrin) | 8.37E−04 | 1.18 | 0.97 | 71 | MAA and Josa vs. Clari, Roxi |
| 47446 | RANBP1 | RAN binding protein 1 | 8.61E−04 | 1.04 | 0.89 | 72 | MAA and Josa vs. Clari, Roxi |
| 47694 | HSPA9B | heat shock 70 kD protein 9B (mortalin-2) | 8.61E−04 | 1.09 | 0.95 | 72 | MAA and Josa vs. Clari, Roxi |
| 46648 | ARHGAP1 | Rho GTPase activating protein 1 | 8.61E−04 | 1.07 | 0.96 | 72 | MAA and Josa vs. Clari, Roxi |
| 49787 | CYC1 | cytochrome c-1 | 8.97E−04 | 1.09 | 0.94 | 72 | MAA and Josa vs. Clari, Roxi |
| 47988 | RPN2 | ribophorin II | 8.97E−04 | 0.93 | 1.02 | 72 | MAA and Josa vs. Clari, Roxi |
| 46923 | PACE | paired basic amino acid cleaving enzyme (furin, | 8.97E−04 | 1.00 | 1.09 | 72 | MAA and Josa vs. Clari, Roxi |
| | | membrane associated receptor protein) | | | | | |
| 49947 | RFC1 | replication factor C (activator 1) 1 (145 kD) | 9.29E−04 | 1.05 | 0.93 | 66 | MAA and Josa vs. Clari, Roxi |
| 48732 | RALA | v-ral simian leukemia viral oncogene homolog A | 9.36E−04 | 0.98 | 1.06 | 72 | MAA and Josa vs. Clari, Roxi |
| | | (ras related) | | | | | |
| 47973 | USF2 | upstream transcription factor 2, c-fos interacting | 9.46E−04 | 0.96 | 1.05 | 70 | MAA and Josa vs. Clari, Roxi |
| 47352 | ADAM11 | a disintegrin and metalloproteinase domain 11 | 9.48E−04 | 0.93 | 1.10 | 45 | MAA and Josa vs. Clari, Roxi |
| 50351 | CFL1 | cofilin 1 (non-muscle) | 9.75E−04 | 1.16 | 1.00 | 72 | MAA and Josa vs. Clari, Roxi |

APPENDIX F
Group of genes that affect inflammation in the lung*

| | | | | +/−Clari, | +LPS +/− Clari, |
|---|---|---|---|---|---|
| | | | | wtd | wtd |
| SpotID | GeneSymbol | GeneName | GeneAssignment | means | means |
| 47876 | SERPINB3 | | serine (or cysteine) proteinase inhibitor, clade B | 1.58 | 1.63 |
| | | | (ovalbumin), member 3 | | |
| 30419 | MMP12 | MMP12 | matrix metalloproteinase 12 (macrophage elastase) | 1.00 | 0.47 |
| 48003 | MMP19 | MMP19 | matrix metalloproteinase 19 | 0.69 | 0.60 |
| 48980 | MMP9 | MMP9, gelatinase B | matrix metalloproteinase 9 (gelatinase B, 92 kD | 0.84 | 0.89 |
| | | | gelatinase, 92 kD type IV collagenase) | | |
| 9206 | CTSC | | cathepsin C | 0.90 | 0.82 |
| 10515 | CTSK | | cathepsin K (pycnodysostosis) | 0.82 | 0.62 |
| 11956 | CXCL10 | IP-10 | small inducible cytokine subfamily B (Cys-X-Cys), | 1.13 | 0.69 |
| | | | member 10 | | |
| 11819 | CXCL11 | I-TAG | small inducible cytokine subfamily B (Cys-X-Cys), | 0.96 | 0.78 |
| | | | member 11 | | |
| 48570 | CXCL9 | monokine induced by | monokine induced by gamma interferon | 1.09 | 0.47 |
| | | gamma interferon | | | |
| 28201 | CCL18 | DC-CK1 | small inducible cytokine subfamily A (Cys—Cys), | 0.89 | 0.68 |
| | | | member 18, pulmonary and activation-regulated | | |

*From Appendix C. For those having a plurality of SPOTIDs, a representative ID is shown.

We claim:

1. A method of screening test macrolides to identify potential anti-inflammatory agents, comprising:
1) contacting a first biological sample with a test macrolide; and
2) detecting, as an alteration, a first level of expression or activity of a target molecule involved in inflammation in the first biological sample relative to a second level of expression or activity of the target molecule involved in inflammation in a second biological sample that has not been contacted with the test macrolide.

2. The method of claim 1, wherein a test macrolide that alters the first level of alteration of expression or activity of the target molecule involved in inflammation relative to the second level of alteration of expression or activity of the target molecule involved in inflammation identifies the test macrolide as a potential anti-inflammatory agent.

3. The method of claim 1, wherein the alteration of the target molecule in said 2) is an alteration in the level of de novo arachidonic acid biosynthesis.

4. The method of claim 3, wherein a macrolide compound that increases or decreases the level of de novo arachidonic acid biosynthesis is selected.

5. The method of claim 4, wherein the level of de novo arachidonic acid biosynthesis is detected by measuring incorporation of labeled arachidonic acid into phospholipids.

6. The method of claim 4, wherein the level of de novo arachidonic acid biosynthesis is detected by measuring incorporation of label into an intermediate in an arachidonic acid biosynthesis pathway.

7. The method of claim 1, wherein the alteration of the target molecule in said 2) is an alteration in the level of extracellular matrix decomposition.

8. The method of claim 7, wherein a macrolide compound that decreases the level of extracellular matrix decomposition is selected.

9. The method of claim 1, wherein the alteration of the target molecule in said 2) is an alteration in the leucocyte infiltration number.

10. The method of claim 9, wherein a macrolide compound that decreases the leucocyte infiltration number is selected.

11. The method of claim 1, wherein the alteration of the target molecule in said 2) is an alteration in the level of expression of a gene involved in arachidonic acid biosynthesis or activation.

12. The method of claim 11, wherein a macrolide compound that increases or decreases the level of expression of said gene is selected.

13. The method of claim 11, wherein said gene is at least one type selected from the group consisting of A5 fatty acid desaturase (FADS1), A6 fatty acid desaturase (FADS2), fatty acid elongase, arachidonyl CoA synthetase, cyclooxygenase 2, prostaglandin E receptor EP4, cytosolic phospholipase A2, fatty acid desaturase, prostaglandin E synthase, prostaglandin E receptor EP1, prostaglandin 12 receptor, and secretory phospholipase A2 receptor 1.

14. The method of claim 1, wherein the alteration of the target molecule in said 2) is an alteration in the level of expression of a gene involved in extracellular matrix decomposition.

15. The method of claim 14, wherein a macrolide compound that increases the level of expression of said gene is selected.

16. The method of claim 15, wherein said gene is at least one type selected from the group consisting of serine proteinase inhibitor, clade B (ovalbumin), and member 3 (SERPINB3).

17. The method of claim 14, wherein a macrolide compound that decreases the level of expression of said gene is selected.

18. The method of claim 17, wherein said gene is at least one type selected from the group consisting of matrix metalloproteinase 12 (MMP12), matrix metalloproteinase 19 (MMP19), matrix metalloproteinase 9 (MMP9), cathepsin C, and cathepsin K.

19. The method of claim 1, wherein the alteration of the target molecule in said 2) is an alteration in the level of expression of a gene involved in leucocyte infiltration.

20. The method of claim 19, wherein a macrolide compound that decreases the level of expression of said gene is selected.

21. The method of claim 19, wherein said gene is at least one type selected from the group consisting of small inducible cytokine subfamily A (Cys-Cys) member 18 (CCL18), small inducible cytokine subfamily B (Cys-X-Cys) member 10 (CXCL10), small inducible cytokine subfamily B (Cys-X-Cys) member 11 (CXCL11), and small inducible cytokine subfamily B (Cys-X-Cys) member 9 (CXCL9).

22. The method of claim 19, wherein expression of the gene is determined by detecting mRNA, protein or protein synthesis.

23. The method of claim 1, wherein the alteration of the target molecule in said 2) is an alteration in the activity of an enzyme involved in arachidonic acid biosynthesis.

24. The method of claim 23, wherein a macrolide compound that increases or decreases the activity of said enzyme is selected.

25. The method of claim 24, wherein said enzyme is at least one type selected from the group consisting of A5 fatty acid desaturase (FADS1), A6 fatty acid desaturase (FADS2), fatty acid elongase, and arachidonyl CoA synthetase.

26. The method of claim 1, wherein the alteration of the target molecule in said 2) is an alteration in the activity of an enzyme involved in extracellular matrix decomposition.

27. The method of claim 26, wherein a macrolide compound that increases the activity of said enzyme is selected.

28. The method of claim 27, wherein said enzyme is at least one type selected from the group consisting of serine proteinase inhibitor, clade B (ovalbumin), and member 3 (SERPINB3).

29. The method of claim 26, wherein a macrolide compound that decreases the activity of said enzyme is selected.

30. The method of claim 29, wherein said enzyme is at least one type selected from the group consisting of matrix metalloproteinase 12 (MMP12), matrix metalloproteinase 19 (MMP19), matrix metalloproteinase 9 (MMP9), cathepsin C, and cathepsin K.

31. The method of claim 1, wherein the alteration of the target molecule in said 2) is an alteration in the activity of a chemokine involved in leucocyte infiltration.

32. The method of claim 31, wherein a macrolide compound that decreases the activity of said chemokine is selected.

33. The method of claim 32, wherein said chemokine is at least one type selected from the group consisting of small inducible cytokine subfamily A (Cys-Cys) member 18 (CCL18), small inducible cytokine subfamily B (Cys-X-Cys) member 10 (CXCL10), small inducible cytokine subfamily B (Cys-X-Cys) member 11 (CXCL11), and small inducible cytokine subfamily B (Cys-X-Cys) member 9 (CXCL9).

34. The method of claim 1, wherein the biological sample comprises cells.

35. The method of claim 1, wherein the biological sample comprises a cellular extract.

36. The method of claim 1, wherein said contacting in said 1) is carried out in vitro.

37. The method of claim 1, wherein said contacting in said 1) is carried out in vivo.

38. The method of claim 1, wherein said contacting in said 1) is carried out in a cell culture.

39. The method of claim 38, wherein the cell culture comprises an epithelial cell line.

40. The method of claim 38, wherein the epithelial cell line is a macrophage cell line.

41. The method of claim 39, wherein the epithelial cell line is A549.

42. The method of claim 40, wherein the epithelial cell line is THP-1.

43. The method of claim 1, wherein said anti-inflammatory action is an action against inflammation in the lung.

44. The method of claim 43, wherein said inflammation in the lung is pulmonary emphysema, bronchitis, pulmonary fibrosis, acute lung injury, viral pneumonia, bacterial pneumonia, or acute respiratory disease syndrome (ARDS).

45. The method of any one of claims 14, wherein expression of the gene is determined by detecting mRNA, protein or protein synthesis.

46. The method of any one of claims 11, wherein expression of the gene is determined by detecting mRNA, protein or protein synthesis.
